(19) United States
(12) Patent Application Publication
(10) Pub. No.: US 2025/0011281 A1
(43) Pub. Date: Jan. 9, 2025

(54) THERAPEUTIC ALKALOID COMPOUNDS

(71) Applicant: Sensorium Therapeutics, Inc., Boston, MA (US)

(72) Inventors: Jacob M. Hooker, Winchester, MA (US); Michael S. Placzek, Tewksbury, MA (US)

(21) Appl. No.: 18/812,309

(22) Filed: Aug. 22, 2024

Related U.S. Application Data
(63) Continuation of application No. PCT/US24/22233, filed on Mar. 29, 2024.
(60) Provisional application No. 63/456,210, filed on Mar. 31, 2023.

(51) Int. Cl. C07D 209/12; A61K 31/404; C07D 405/04

(52) U.S. Cl. CPC C07D 209/12; A61K 31/404; C07D 405/04

(57) ABSTRACT

Disclosed are compounds that are derivatives of mesembranol or mesembrenol, and related methods of preparing and using these compounds. The disclosed compounds may inhibit SERT.

### RELATED APPLICATIONS

This patent application claims the benefit of priority to U.S. Provisional Patent Application No. 63/456,210 filed Mar. 31, 2023; which is incorporated by reference in its entirety.

### TECHNICAL FIELD

The present disclosure relates to the field of medicine, including the discovery of alkaloid compounds useful for eliciting antidepressant and/or anxiolytic effects by inhibiting, in part, the serotonin transporter protein (5-HTT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol, among the alkaloids shown below, are present in Sceletium tortuosum extracts used for treatment of anxiety, stress and mental health conditions. Mesembranol has lower concentrations in the extracts compared to the other alkaloids.

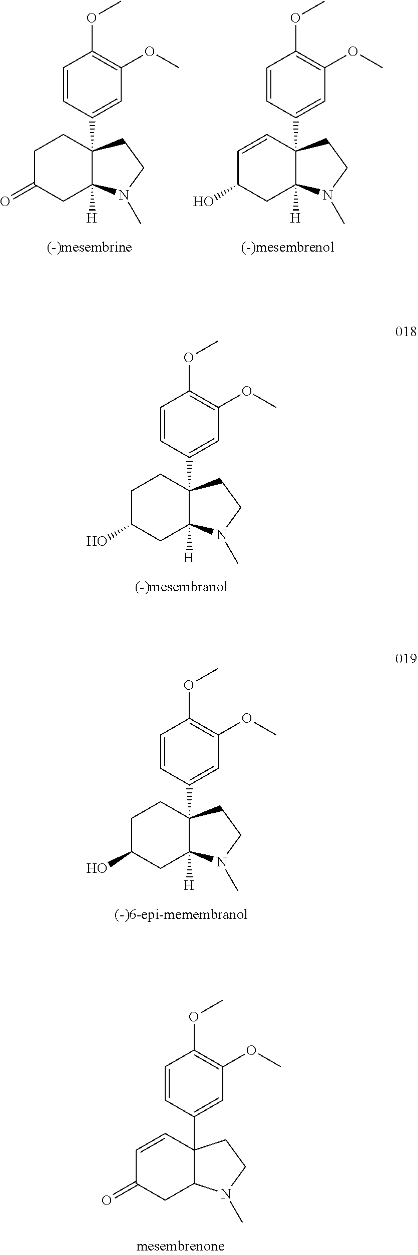 [SMILES: COc1ccc([C@@]23CCC(=O)C[C@@H]2N(C)CC3)cc1OC ; COc1ccc([C@@]23C=C[C@@H](O)C[C@@H]2N(C)CC3)cc1OC ; COc1ccc([C@@]23CC[C@@H](O)C[C@@H]2N(C)CC3)cc1OC ; COc1ccc([C@@]23CC[C@H](O)C[C@@H]2N(C)CC3)cc1OC ; COc1ccc(C23C=CC(=O)CC2N(C)CC3)cc1OC]

An analysis of a standardized commercial extract of Sceletium tortuosum was reported in 2011 (obtained as a product under the tradename, Zembrin®) as having 0.35%-0.45% total alkaloids, with mesembrenone and mesembrenol comprising ≥60%, and mesembrine contributing <20% (See Harvey et al., “Pharmacological actions of the South African medicinal and functional food plant Sceletium tortuosum and its principal alkaloids,” Journal of Ethnopharmacology 137 (2011) 1124-11292011 and Murbach et. al., “A toxicological safety assessment of a standardized extract of Sceletium tortuosum (Zembrin®) in rats,” Food and Chemical Toxicology 74 (2014) 190-199). The extract gave >80% inhibition at serotonin (5-HT) transporter with potency of the isolated alkaloids at the 5-HT transporter reported as shown in Table A below (Harvey et al., 2011). Referring to the data in Table A, concentration-dependent inhibition was found, with mesembrine being the more active compound (i.e., 20 times more potent than mesembrenone and 87 times more active than mesembrenol) in the 5-HT transporter assay. A toxicological safety assessment of this standardized extract was subsequently reported in 2014 (Murbach et al., 2014).

TABLE A
Summary of analysis of the concentration response curves of alkaloids
on binding to the 5-HT transporter (Harvey et al., 2011)

5-HT transporter (SERT)

| Compound | Ki (nM) | nH |
|---|---|---|
| Mesembrine | 1.4 | 1.0 |
| Mesembrenone | 27 | 1.0 |
| Mesembrenol | 62 | 1.1 |

However, bioactive plant extracts for therapeutic consumption can vary widely both seasonally and between different Sceletium tortuosum plants, and fail to provide a sufficiently reproducible and stable phytochemical profile of desired biologically active components. Plants of the genus Sceletium and extracts thereof can vary widely in terms of the total alkaloid content, as well as the chemistry and relative concentrations of individual Sceletium plant derived alkaloids. In addition, it has been reported that mesembranol concentrations in sceletium tortuosum can vary across regions of South Africa, and are relatively low in most plant extracts that were tested. Lastly, sceletium alkaloids may be unstable under a variety of conditions that can occur during extraction from plant material, as well as during storage and formulation of the extract.

In Sceletium tortuosum extract, mesembranol has low concentrations compared to the other major alkaloids, and 6-epi-mesembranol is only detected in trace amounts. The therapeutic use of mesembranol and 6-epi-mesembranol has been limited by the abundance, variability, and instability of these alkaloids in natural extract products, and the instability and pharmacokinetic profile of these compounds as obtained from natural products. Lastly, reported plasma concentrations of sceletium tortuosum alkaloids in a rodent pharmacokinetic study were low.

There remains an unmet need for new compounds inhibiting the serotonin transporter protein (5-HTT).

### SUMMARY

Described herein are compounds Formula (IA), (IB) or (IC)

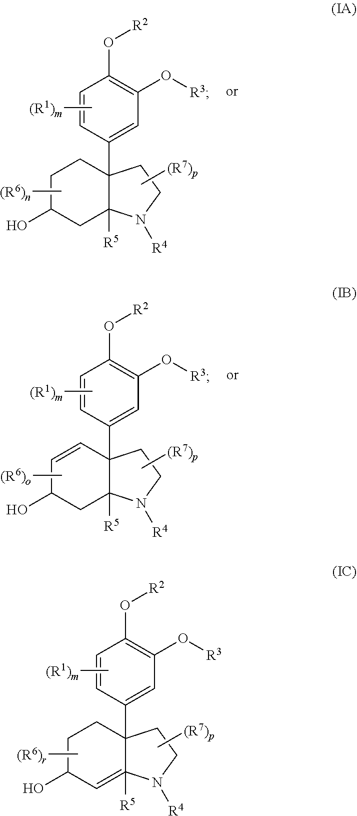

or a pharmaceutically acceptable salt thereof; wherein
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R⁴ is H or alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a};
R⁵ is H, deuterium, or methyl;
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or two R's on a single carbon atom together with the carbon atom to which they are attached combine to form cycloalkyl, heterocyclyl or heteroaryl, wherein each hydrogen atom in cycloalkyl, heterocyclyl, and 5 heteroaryl is optionally substituted by halo, hydroxy, alkoxy, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, alkyl, —OH, or —NH₂; wherein each hydrogen atom in alkyl is optionally substituted by —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl;
provided the compound is not:

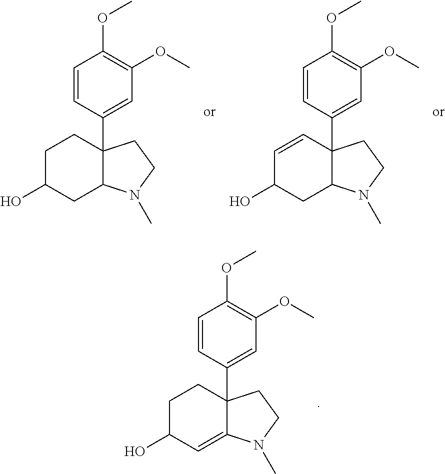 [SMILES: COc1ccc(C23CCC(O)CC2N(C)CC3)cc1OC ; "or" ; COc1ccc(C23C=CC(O)CC2N(C)CC3)cc1OC ; "or" ; COc1ccc(C23CCC(O)C=C2N(C)CC3)cc1OC]

In certain embodiments, the compound is a compound of Formula (IA), (IB) or (IC):

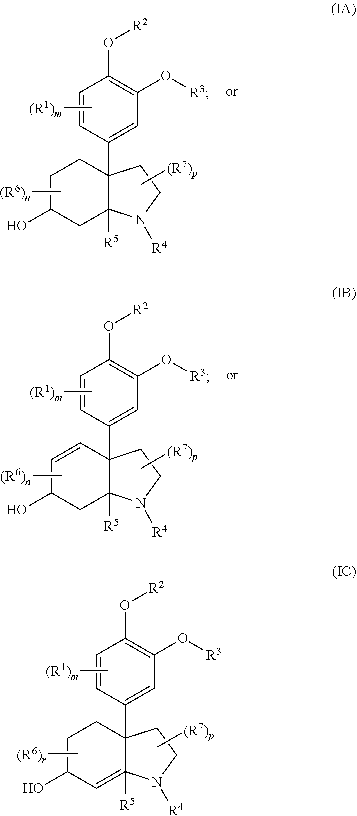

or a pharmaceutically acceptable salt thereof; wherein
each R¹ is independently halo, deuterium, C₁₋₄ alkyl, C₂₋₄ alkenyl, C₂₋₄ alkynyl, C₃₋₈ cycloalkyl, C₃₋₈ cycloalkenyl, 3-8 membered heterocyclyl comprising one or more N, O or S heteroatoms, C₅₋₆ aryl, 5-6 membered heteroaryl comprising one or more N, O or S heteroatoms, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, and alkynyl, is optionally substituted by halo, C₁₋₄ alkyl, C₁₋₄ alkanol, C₅₋₆ aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; and wherein each hydrogen atom in cycloalkyl, cycloalkenyl, heterocyclyl, aryl, and heteroaryl is optionally substituted by halo, C₁₋₄ alkyl, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently C₁₋₄ alkyl, H, C₃₋₈ cycloalkyl, C₂₋₄ alkenyl, C₂₋₄ alkynyl, or C₅₋₆ aryl; wherein each hydrogen atom in alkyl, alkenyl, and alkynyl, is optionally substituted by halo, deuterium, C₃₋₈ cycloalkyl, C₅₋₆ aryl, or OR^{a}; and wherein each hydrogen atom in cycloalkyl, and aryl is optionally substituted by halo, deuterium, C₁₋₄ alkyl, C₁₋₄ haloalkyl or OR^{a}; or
R² and R³ together with the atoms to which they are attached combine to form 3-8 membered heterocyclyl comprising one or more N, O or S heteroatoms, or 5-6 membered heteroaryl comprising one or more N, O or S heteroatoms, wherein each hydrogen atom in heterocyclyl and heteroaryl is optionally substituted by halo, C₁₋₄ alkyl, C₁₋₄ haloalkyl or OR^{a};
R⁴ is H or C₁₋₄ alkyl, wherein each hydrogen atom in alkyl is optionally substituted by halo, deuterium, C₃₋₈ cycloalkyl, C₅₋₆ aryl, —OR^{a}, or —NR^{a}R^{b}; wherein each hydrogen atom in aryl is optionally substituted by OR^{a};
R⁵ is H, deuterium, or methyl;
each R⁶ is independently C₁₋₄ alkyl, deuterium, C₃₋₈ cycloalkyl, —Si(C₁₋₄ alkyl)₃, —C(O)—(C₁₋₄)-alkyl, C₅₋₆ aryl, 5-6 membered heteroaryl comprising one or more N, O or S heteroatoms, or 3-8 membered heterocyclyl comprising one or more N, O or S heteroatoms; wherein each hydrogen atom in alkyl, —Si(alkyl)₃, and —C(O)alkyl is optionally substituted by halo, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; and wherein each hydrogen atom in cycloalkyl, phenyl, heteroaryl, and heterocyclyl is optionally substituted by halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form C₅₋₆ aryl, C₃₋₈ cycloalkyl, 5-6 membered heteroaryl comprising one or more N, O or S heteroatoms, or 3-8 membered heterocyclyl comprising one or more N, O or S heteroatoms;
or two R⁶s on different carbon atoms together with the carbon atoms to which they are attached combine to form a C₃₋₈ cycloalkyl, 3-8 membered heterocyclyl comprising one or more N, O or S heteroatoms, or a 5-6 membered heteroaryl comprising one or more N, O or S heteroatoms, wherein each hydrogen atom in cycloalkyl, heterocyclyl, and heteroaryl is optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form C₃₋₈ cycloalkyl, 3-8 membered heterocyclyl comprising one or more N, O or S heteroatoms, or 5-6 membered heteroaryl comprising one or more N, O or S heteroatoms, wherein each hydrogen atom in cycloalkyl, heterocyclyl, and 5-6 membered heteroaryl comprising one or more N, O or S heteroatoms is optionally substituted by halo, hydroxy, C₁₋₄ alkoxy, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, C₁₋₄ alkyl, C₁₋₄ haloalkyl, —OH, or —NH₂; wherein each hydrogen atom in alkyl is optionally substituted by —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
each R^{a} and R^{b} is independently H, C₁₋₄ alkyl, C₂₋₄ alkenyl, C₃₋₈ cycloalkyl, 3-8 membered heterocyclyl comprising one or more N, O or S heteroatoms, C₅₋₆ aryl, or 5-6 membered heteroaryl;
or if an instance of R¹ is —NR^{a}R^{b}, then R^{a} and R^{b} may combine with the nitrogen atom to which they are attached to form 3-8 membered heterocyclyl or 5-6 membered heteroaryl; wherein each hydrogen atom in alkyl, or alkenyl, is optionally substituted by halo, hydroxy, C₁₋₄ alkyl, C₁₋₄ alkanol, C₅₋₆ aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; wherein each hydrogen atom in cycloalkyl, heterocyclyl, aryl, or heteroaryl is optionally substituted by halo, hydroxy, C₁₋₄ alkyl, C₁₋₄ haloalkyl, C₁₋₄ alkanol, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, C₁₋₄ alkyl, C₂₋₄ alkenyl, C₃₋₈ cycloalkyl, 3-8 membered heterocyclyl comprising one or more N, O or S heteroatoms, C₅₋₆ aryl, or 5-6 membered heteroaryl comprising one or more N, O or S heteroatoms;
provided the compound is not:

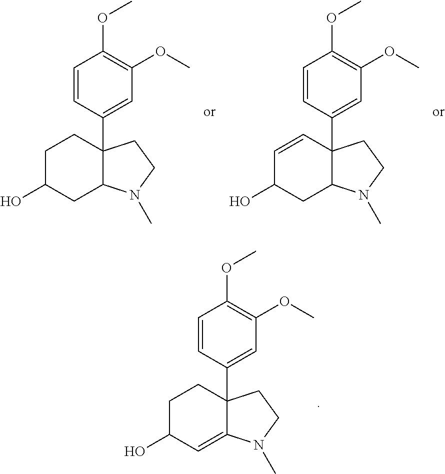 [SMILES: COc1ccc(C23CCC(O)CC2N(C)CC3)cc1OC ; "or" ; COc1ccc(C23C=CC(O)CC2N(C)CC3)cc1OC ; "or" ; COc1ccc(C23CCC(O)C=C2N(C)CC3)cc1OC]

In certain embodiments, the compound is a compound of Formula (IA), (IB) or (IC), wherein R⁴ is C₁₋₄ alkyl; R⁵ is H; p is 0; and each R^{a} and R^{b} is independently H, or C₁₋₄ alkyl.

In certain embodiments, the compound is a compound of Formula (IA), (IB) or (IC) that is also a compound of Formula (IA-c), (IB-c) and (IC-c), or a pharmaceutically acceptable salt thereof,

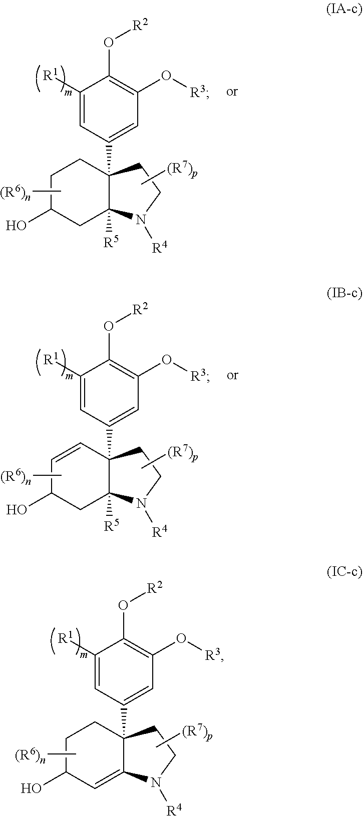

wherein each R¹ is independently halo, C₁₋₄ alkyl, C₁₋₄ haloalkyl, C₂₋₄ alkenyl, —OR^{a}, —NR^{a}R^{b}, —CHO, —C(O)R^{a}, —CO₂R^{a}, —C(O)NR^{a}R^{b}, —CN, or C₃₋₆ cycloalkyl; m is 0 or 1; and n is 0, 1, or 2.

In certain embodiments, the compound is a compound of Formula (IA), (IB) or (IC), wherein each R¹ is independently halo, C₁₋₄ alkyl, C₁₋₄ haloalkyl, —CH═CH₂, —OCH₃, —NR^{a}R^{b}, —CHO, —C(O)R^{a}, —CO₂R^{a}, —C(O)NR^{a}R^{b}, —CN, or cyclopropyl; and R^{a} and R^{b} are each independently H or methyl. In certain embodiments, the compound is a compound of Formula (IA), (IB) or (IC), wherein n is 1 or 2 and each R⁶ is independently C₁₋₄ alkyl.

In certain embodiments, the compound is a compound of Formula (IA), (IB) or (IC), wherein each R⁶ is independently methyl. In certain embodiments, the compound is a compound of Formula (IA), (IB) or (IC), wherein two R⁶s on a single carbon atom together with the carbon atom to which they are attached combine to form a spirocyclic C₃₋₆ cycloalkyl or a 3-6 membered heterocyclyl comprising one or more N, O or S heteroatoms.

In certain embodiments, the compound is a compound of Formula (IA), (IA-c), (IB), (IB-c), (IC), or (IC-c) wherein two R⁶s on a single carbon atom together with the carbon atom to which they are attached combine to form a spirocyclic cyclopropyl.

In certain embodiments, the compound is a compound of Formula (IA), (IA-c), (IB), (IB-c), (IC), or (IC-c), wherein two R's on different adjacent carbon atoms together with the carbon atoms to which they are attached combine to form a C₅₋₈ cycloalkyl, 5-8 membered heterocyclyl comprising one or more N, O or S heteroatoms. In certain embodiments, the compound is a compound of Formula (IA), (IB) or (IC), wherein two R's on different adjacent carbon atoms together with the carbon atoms to which they are attached combine to form an epoxide.

In certain embodiments, the compound is a compound of Formula (IA), or (IA-c), wherein two R's on different carbon atoms together with the carbon atoms to which they are attached combine to form a bridging C₃₋₈ cycloalkyl, 3-8 membered heterocyclyl comprising one or more N, O or S heteroatoms, wherein each hydrogen atom in cycloalkyl, or heterocyclyl, is optionally substituted by halo, hydroxy, C₁₋₄ alkyl, C₁₋₄ haloalkyl, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, the compound is a compound of Formula (IA), or (IA-c), wherein the compound is a compound of formula (VIII) or a pharmaceutically acceptable salt thereof

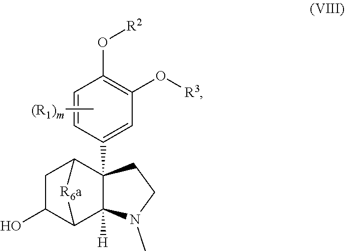

wherein each R¹ is independently halo, C₁₋₄ alkyl, C₂₋₄ alkenyl, —OR^{a}, —NR^{a}R^{b}, —CHO, —C(O)R^{a}, —CO₂R^{a}, —C(O)NR^{a}R^{b}, —CN, C₃₋₆ cycloalkyl, C₁₋₄ alkyl, or C₁₋₄ haloalkyl; m is 0 or 1; each R^{a} and R^{b} is independently H or methyl; each of R² and R³ is independently H, C₂₋₄ alkenyl, C₂₋₄ alkynyl, C₃₋₈ cycloalkyl, C₁₋₄ alkyl, C₁₋₄ haloalkyl, OR^{a}, C₃₋₈ cycloalkyl, or phenyl; and R₆ₐ is C₁₋₄ alkyl.

In certain embodiments, the compound is a compound of Formula (IA), (IA-c), (VIII), (IB), (IB-c), (IC), or (IC-c) wherein m is 0, and each of R² and R³ is independently H, C₁₋₄ alkyl, C₁₋₄ alkyl substituted with one or more F, —CH₂CH═CH₂, —CH₂(CCH), C₃₋₆ cycloalkyl, —(CH₂)—(C₃₋₆ cycloalkyl). In certain embodiments, the compound is a compound of Formula (IA), (IA-c), (VIII), (IB), (IB-c), (IC), or (IC-c) wherein each of R² and R³ is independently H, methyl, methyl substituted with one or more F or ethyl.

In certain embodiments, the compound is a compound of formula (VIII) or a pharmaceutically acceptable salt thereof

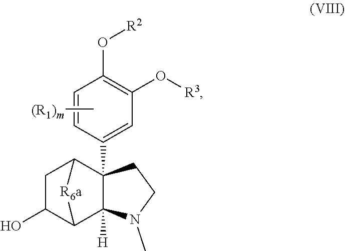

wherein R¹ is halo, methyl, methoxy, cyclopropyl, amido or acetyl; m is 0 or 1; each of R² and R³ is independently H, C₁₋₄ alkyl, or C₁₋₄ haloalkyl; or R² an R³ together with the atoms to which they are attached combine to form a 5-6 membered heterocyclyl comprising two oxygen heteroatoms wherein each hydrogen atom in the heterocyclyl is optionally substituted by halo, C₁₋₄ alkyl, C₁₋₄ haloalkyl or OR^{a}; each R^{a} is independently H or C₁₋₄ alkyl; and R^{6a} is C₁₋₄ alkyl. In certain embodiments, the compound is a compound of formula (VIII) or a pharmaceutically acceptable salt thereof wherein each of R² and R³ is independently H, or C₁₋₄ alkyl optionally substituted with one or more F. In certain embodiments, the compound is a compound of formula (VIII) or a pharmaceutically acceptable salt thereof, wherein R² an R³ together with the atoms to which they are attached combine to form a 5-membered heterocyclyl comprising two oxygen heteroatoms wherein each hydrogen atom in the heterocyclyl is optionally substituted by one or more F.

In certain embodiments, the compound is a compound of Formula (IA), (IA-c), (IB), (IB-c), (IC), or (IC-c) that is also a compound of Formula (IX-A), (IX—B) or (IX—C)

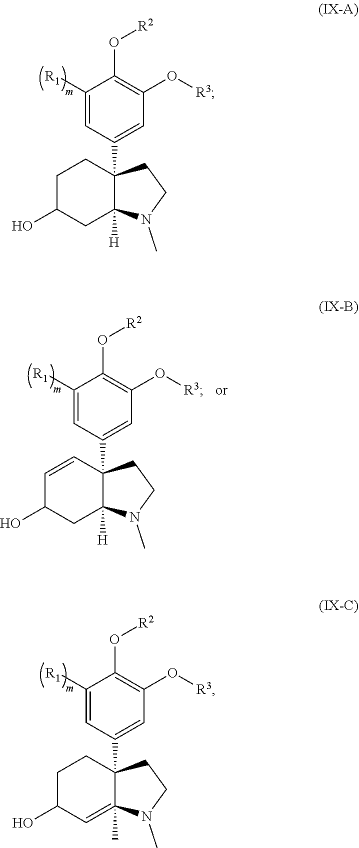

or a pharmaceutically acceptable salt thereof; wherein R¹ is halo, methoxy, cyclopropyl, amido or acetyl; m is 0 or 1; each of R² and R³ is independently H, C₁₋₄ alkyl, or C₁₋₄ haloalkyl; or R² an R³ together with the atoms to which they are attached combine to form a 5-6 membered heterocyclyl comprising two oxygen heteroatoms wherein each hydrogen atom in the heterocyclyl is optionally substituted by halo, C₁₋₄ alkyl, C₁₋₄ haloalkyl or OR^{a}; and each R^{a} is independently H or C₁₋₄ alkyl.

In certain embodiments, the compound is a compound of Formula (IA), (IA-c), (IX-a), (IB), (IB-c), (IX—B), (IC), (IC-c) or (IX—C), wherein each of R² and R³ is independently H, or C₁₋₄ alkyl optionally substituted with one or more F. In certain embodiments, the compound is a compound of Formula (IA), (IA-c), (IX-a), (IB), (IB-c), (IX—B), (IC), (IC-c) or (IX—C), wherein R² an R³ together with the atoms to which they are attached combine to form a 5-membered heterocyclyl comprising two oxygen heteroatoms wherein each hydrogen atom in the heterocyclyl is optionally substituted by one or more F.

In certain embodiments, the compound is a compound of formula (IA-a), (IA-b), (IX-A-a), (IX-A-b), (X-A-a-1) or (X-A-b-1). In certain embodiments, the compound is a compound of formula (IA-a)

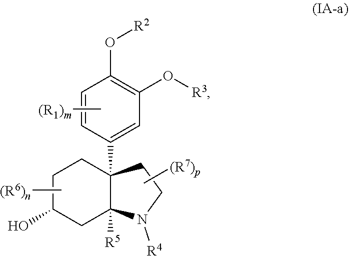

or a pharmaceutically acceptable salt thereof.

In certain embodiments, the compound is a compound of formula (IA-b)

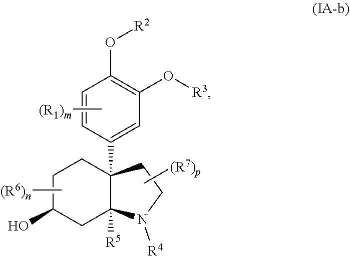

or a pharmaceutically acceptable salt thereof.

In some embodiments, the compound is a compound of formula (IX-A-a)

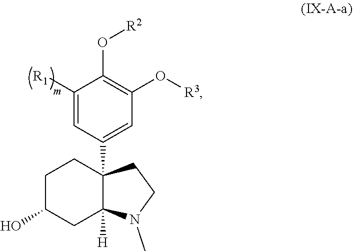

or a pharmaceutically acceptable salt thereof.

In certain embodiments, the compound is a compound of formula (IX-A-b),

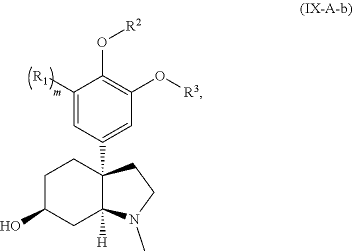

or a pharmaceutically acceptable salt thereof.

In certain embodiments, the compound is a compound of formula (X-A-a-1)

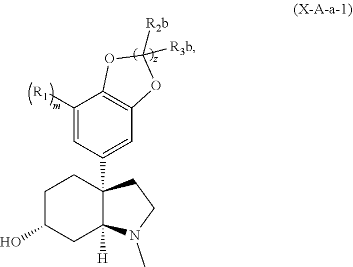

or a pharmaceutically acceptable salt thereof, wherein z is 1 or 2, and R_{2b} and R_{3b} are H or F.

In certain embodiments, the compound is a compound of formula (X-A-b-1)

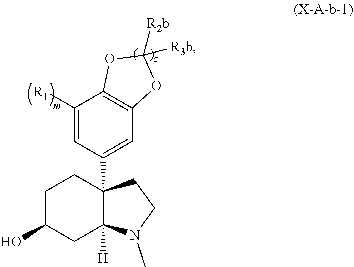

or a pharmaceutically acceptable salt thereof, wherein z is 1 or 2, and R_{2b} and R_{3b} are H or F.

In certain embodiments, the compound is a compound of formula (IB), (IB-a), (IB-b), (X—B-a-1), or (X—B-b-1). In certain embodiments, the compound is a compound of formula (IB-a)

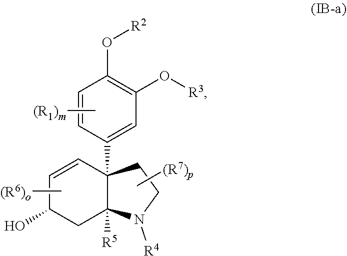

or a pharmaceutically acceptable salt thereof. In certain embodiments, the compound is a compound of formula (IB-b)

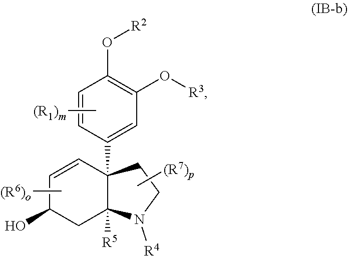

or a pharmaceutically acceptable salt thereof. In certain embodiments, the compound is a compound of formula (X—B-a-1)

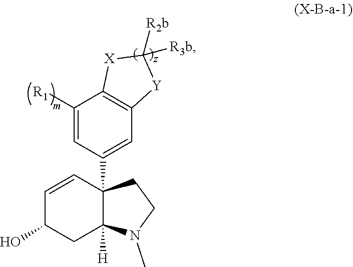

or a pharmaceutically acceptable salt thereof, wherein z is 1 or 2, X and Y are each oxygen, and R_{2b} and R_{3b} are H or F. In certain embodiments, the compound is a compound of formula (X—B-b-1)

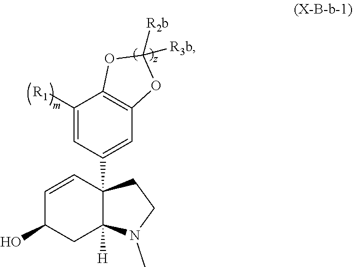

or a pharmaceutically acceptable salt thereof, wherein z is 1 or 2, and R_{2b} and R_{3b} are H or F.

In certain embodiments, the compound is a compound of formula (IC), (IC-a), (IC-b), (X—C-a-1), or (X—C-b-1). In certain embodiments, the compound is a compound of formula (IC-a)

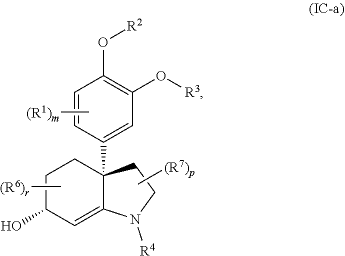

or a pharmaceutically acceptable salt thereof. In certain embodiments, the compound is a compound of formula (IC-b)

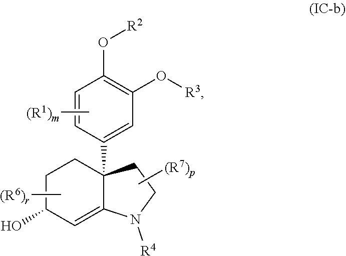

or a pharmaceutically acceptable salt thereof. In certain embodiments, the compound is a compound of formula (X—C-a-1)

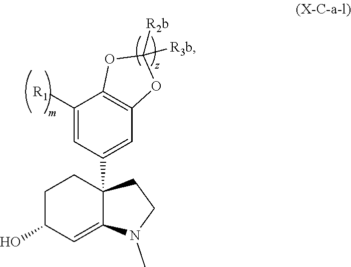

or a pharmaceutically acceptable salt thereof, wherein z is 1 or 2, and R_{2b} and R_{3b} are H or F. In certain embodiments, the compound is a compound of formula (X—C-b-1)

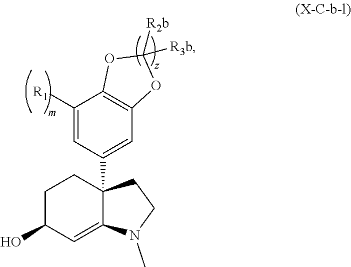

or a pharmaceutically acceptable salt thereof, wherein z is 1 or 2, and R_{2b} and R_{3b} are H or F.

In some embodiments, the compound is a compound selected from:

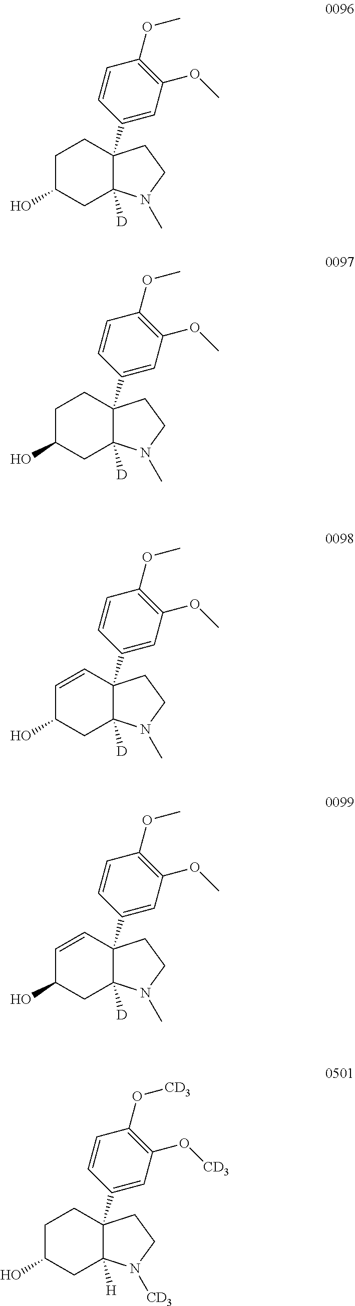 [SMILES: [2H][C@]12C[C@H](O)CC[C@@]1(c1ccc(OC)c(OC)c1)CCN2C ; [2H][C@]12C[C@@H](O)CC[C@@]1(c1ccc(OC)c(OC)c1)CCN2C ; [2H][C@]12C[C@H](O)C=C[C@@]1(c1ccc(OC)c(OC)c1)CCN2C ; [2H][C@]12C[C@@H](O)C=C[C@@]1(c1ccc(OC)c(OC)c1)CCN2C ; [2H]C([2H])([2H])Oc1ccc([C@@]23CC[C@@H](O)C[C@@H]2N(C([2H])([2H])[2H])CC3)cc1OC([2H])([2H])[2H]]

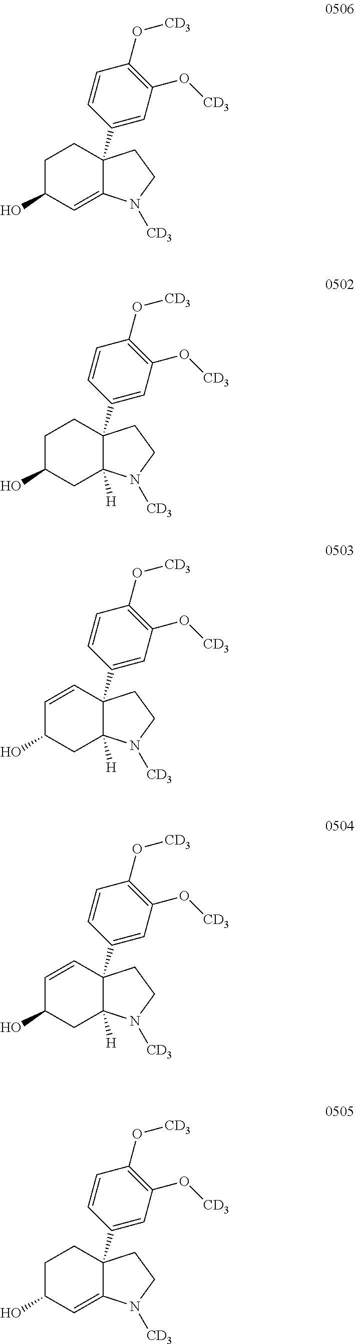 [SMILES: [2H]C([2H])([2H])Oc1ccc([C@@]23CC[C@H](O)C=C2N(C([2H])([2H])[2H])CC3)cc1OC([2H])([2H])[2H] ; [2H]C([2H])([2H])Oc1ccc([C@@]23CC[C@H](O)C[C@@H]2N(C([2H])([2H])[2H])CC3)cc1OC([2H])([2H])[2H] ; [2H]C([2H])([2H])Oc1ccc([C@@]23C=C[C@@H](O)C[C@@H]2N(C([2H])([2H])[2H])CC3)cc1OC([2H])([2H])[2H] ; [2H]C([2H])([2H])Oc1ccc([C@@]23C=C[C@H](O)C[C@@H]2N(C([2H])([2H])[2H])CC3)cc1OC([2H])([2H])[2H] ; [2H]C([2H])([2H])Oc1ccc([C@@]23CC[C@@H](O)C=C2N(C([2H])([2H])[2H])CC3)cc1OC([2H])([2H])[2H]]

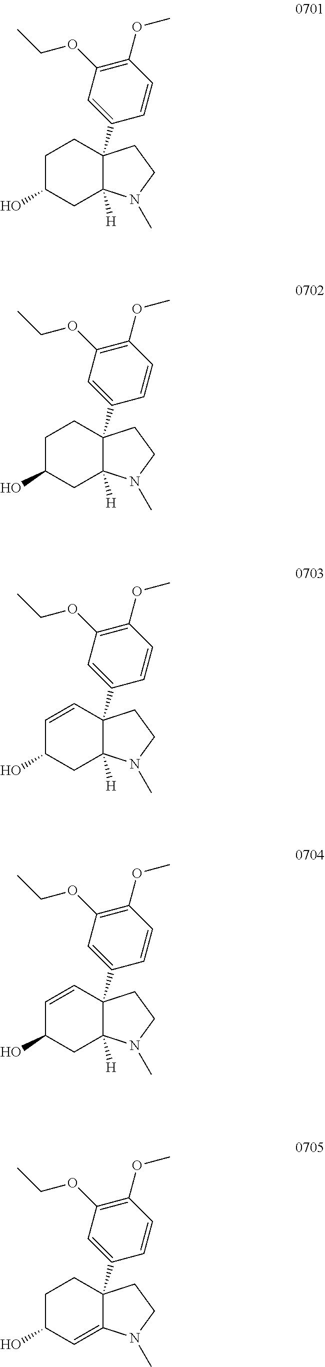 [SMILES: CCOc1cc([C@@]23CC[C@@H](O)C[C@@H]2N(C)CC3)ccc1OC ; CCOc1cc([C@@]23CC[C@H](O)C[C@@H]2N(C)CC3)ccc1OC ; CCOc1cc([C@@]23C=C[C@@H](O)C[C@@H]2N(C)CC3)ccc1OC ; CCOc1cc([C@@]23C=C[C@H](O)C[C@@H]2N(C)CC3)ccc1OC ; CCOc1cc([C@@]23CC[C@@H](O)C=C2N(C)CC3)ccc1OC]

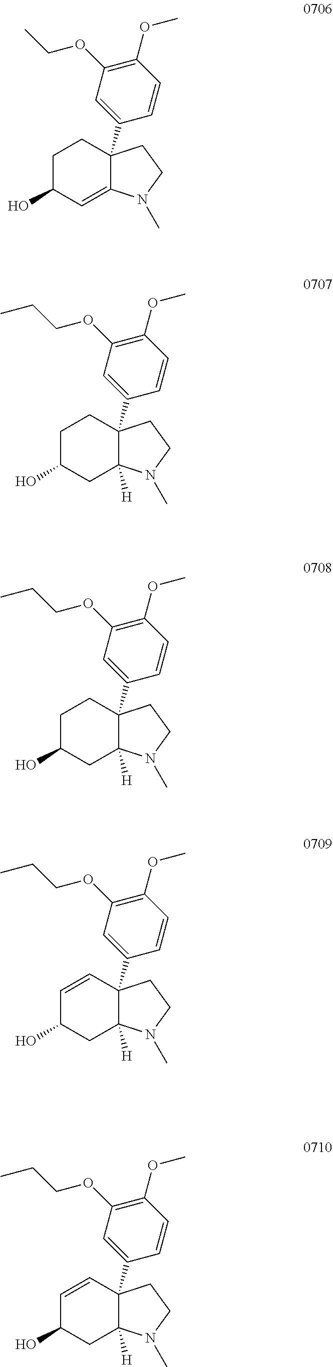

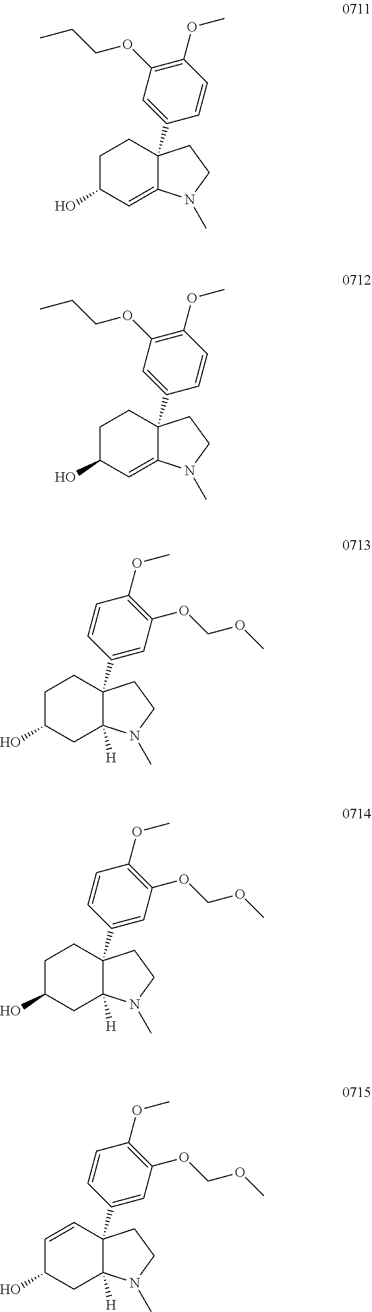

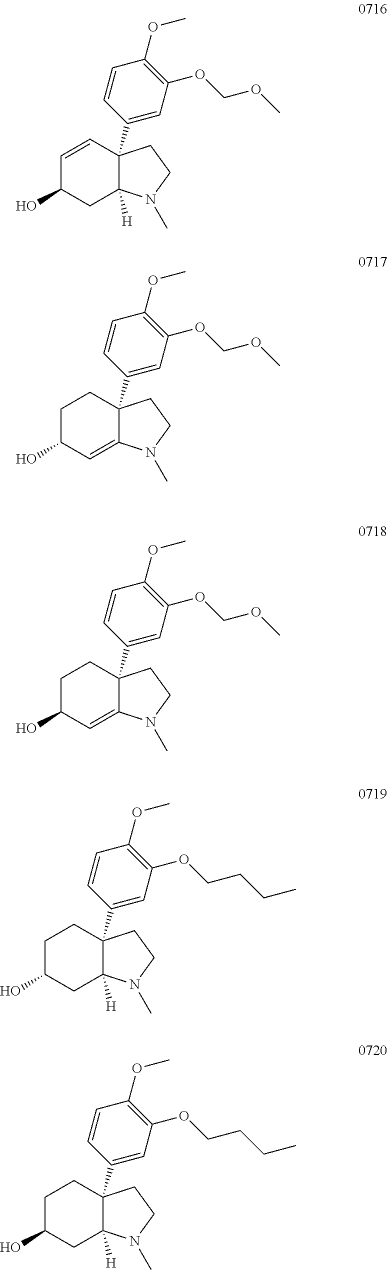 [SMILES: COCOc1cc([C@@]23C=C[C@H](O)C[C@@H]2N(C)CC3)ccc1OC ; COCOc1cc([C@@]23CC[C@@H](O)C=C2N(C)CC3)ccc1OC ; COCOc1cc([C@@]23CC[C@H](O)C=C2N(C)CC3)ccc1OC ; CCCCOc1cc([C@@]23CC[C@@H](O)C[C@@H]2N(C)CC3)ccc1OC ; CCCCOc1cc([C@@]23CC[C@H](O)C[C@@H]2N(C)CC3)ccc1OC]

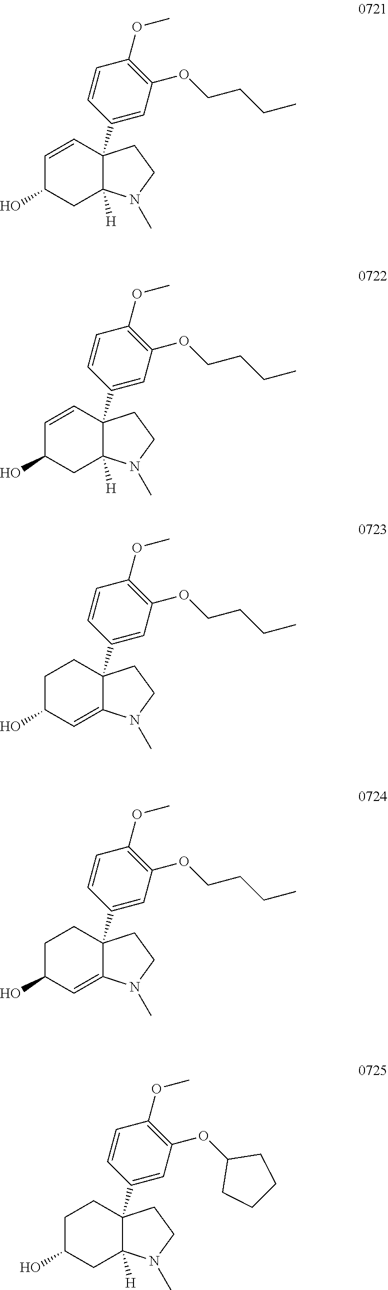 [SMILES: CCCCOc1cc([C@@]23C=C[C@@H](O)C[C@@H]2N(C)CC3)ccc1OC ; CCCCOc1cc([C@@]23C=C[C@H](O)C[C@@H]2N(C)CC3)ccc1OC ; CCCCOc1cc([C@@]23CC[C@@H](O)C=C2N(C)CC3)ccc1OC ; CCCCOc1cc([C@@]23CC[C@H](O)C=C2N(C)CC3)ccc1OC ; COc1ccc([C@@]23CC[C@@H](O)C[C@@H]2N(C)CC3)cc1OC1CCCC1]

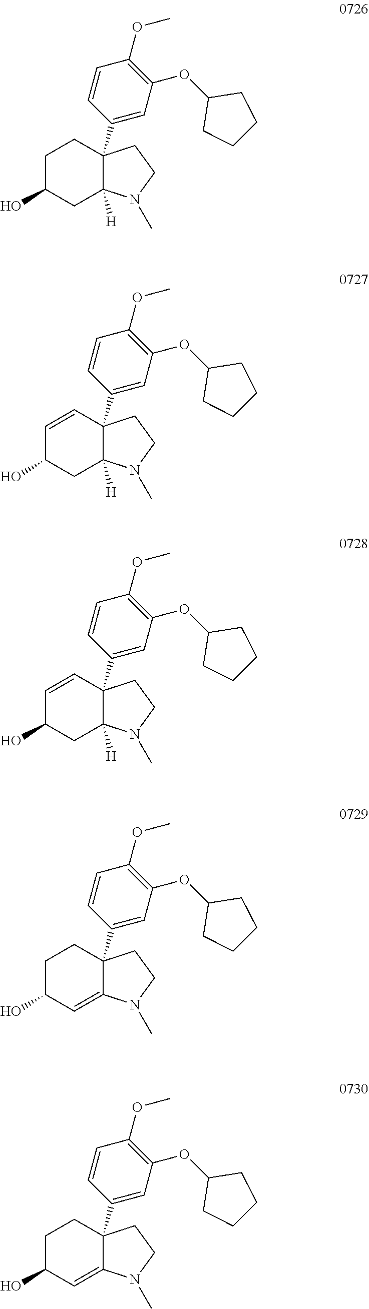 [SMILES: COc1ccc([C@@]23CC[C@H](O)C[C@@H]2N(C)CC3)cc1OC1CCCC1 ; COc1ccc([C@@]23C=C[C@@H](O)C[C@@H]2N(C)CC3)cc1OC1CCCC1 ; COc1ccc([C@@]23C=C[C@H](O)C[C@@H]2N(C)CC3)cc1OC1CCCC1 ; COc1ccc([C@@]23CC[C@@H](O)C=C2N(C)CC3)cc1OC1CCCC1 ; COc1ccc([C@@]23CC[C@H](O)C=C2N(C)CC3)cc1OC1CCCC1]

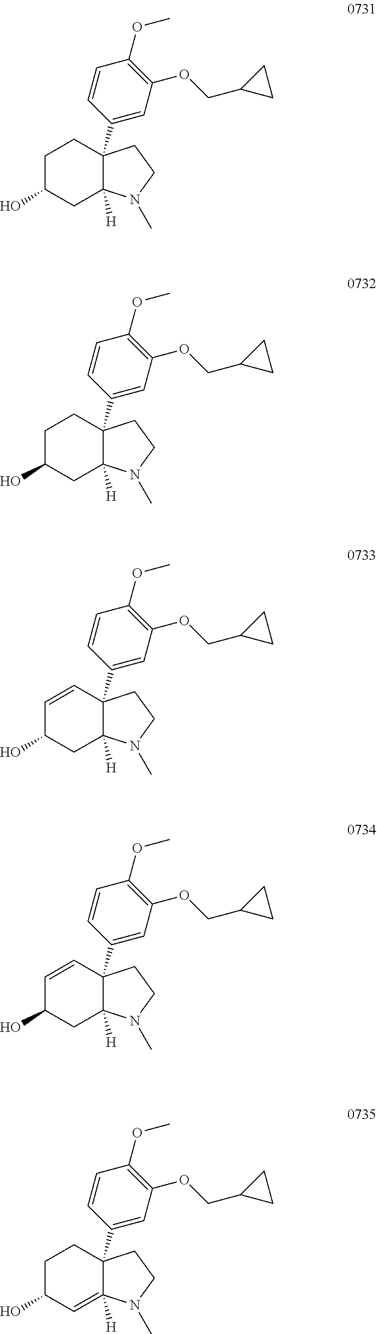

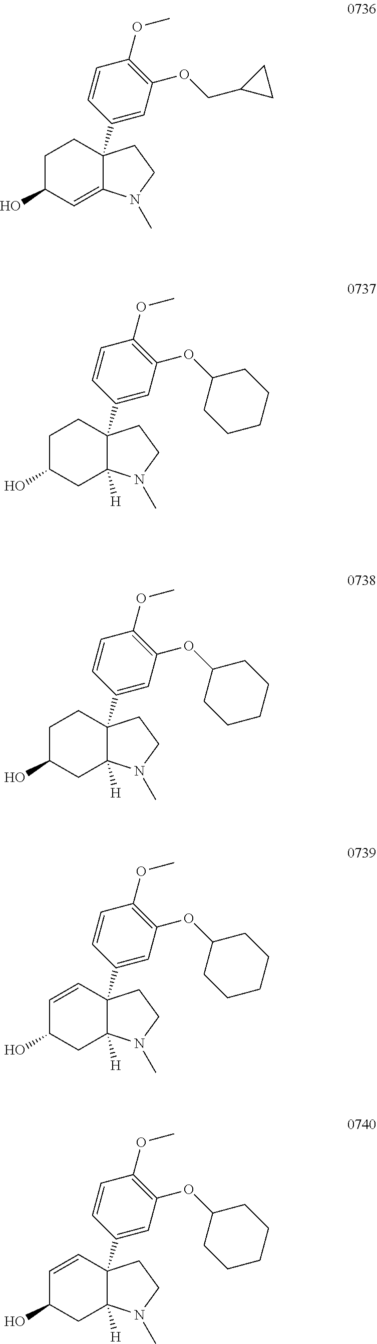 [SMILES: COc1ccc([C@@]23CC[C@H](O)C=C2N(C)CC3)cc1OCC1CC1 ; COc1ccc([C@@]23CC[C@@H](O)C[C@@H]2N(C)CC3)cc1OC1CCCCC1 ; COc1ccc([C@@]23CC[C@H](O)C[C@@H]2N(C)CC3)cc1OC1CCCCC1 ; COc1ccc([C@@]23C=C[C@@H](O)C[C@@H]2N(C)CC3)cc1OC1CCCCC1 ; COc1ccc([C@@]23C=C[C@H](O)C[C@@H]2N(C)CC3)cc1OC1CCCCC1]

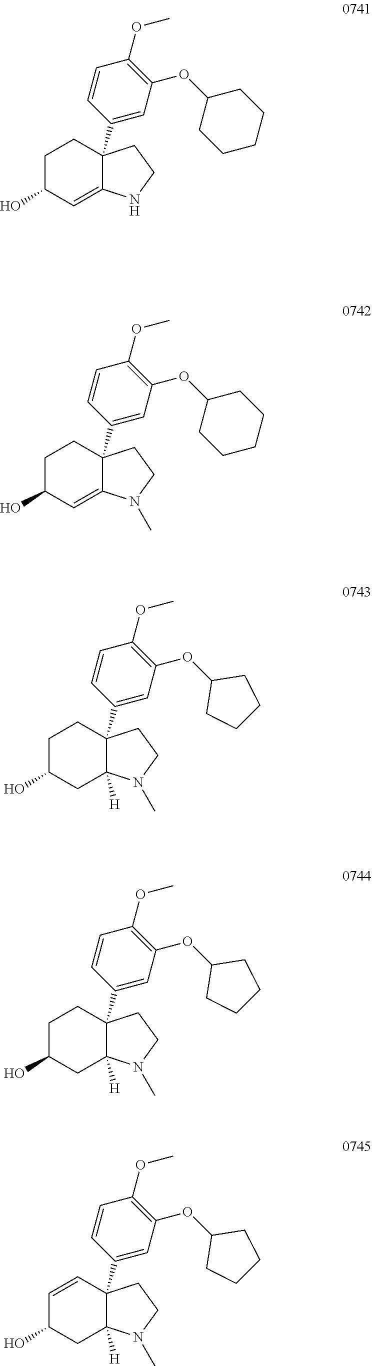 [SMILES: COc1ccc([C@]23CCNC2=C[C@H](O)CC3)cc1OC1CCCCC1 ; COc1ccc([C@@]23CC[C@H](O)C=C2N(C)CC3)cc1OC1CCCCC1 ; COc1ccc([C@@]23CC[C@@H](O)C[C@@H]2N(C)CC3)cc1OC1CCCC1 ; COc1ccc([C@@]23CC[C@H](O)C[C@@H]2N(C)CC3)cc1OC1CCCC1 ; COc1ccc([C@@]23C=C[C@@H](O)C[C@@H]2N(C)CC3)cc1OC1CCCC1]

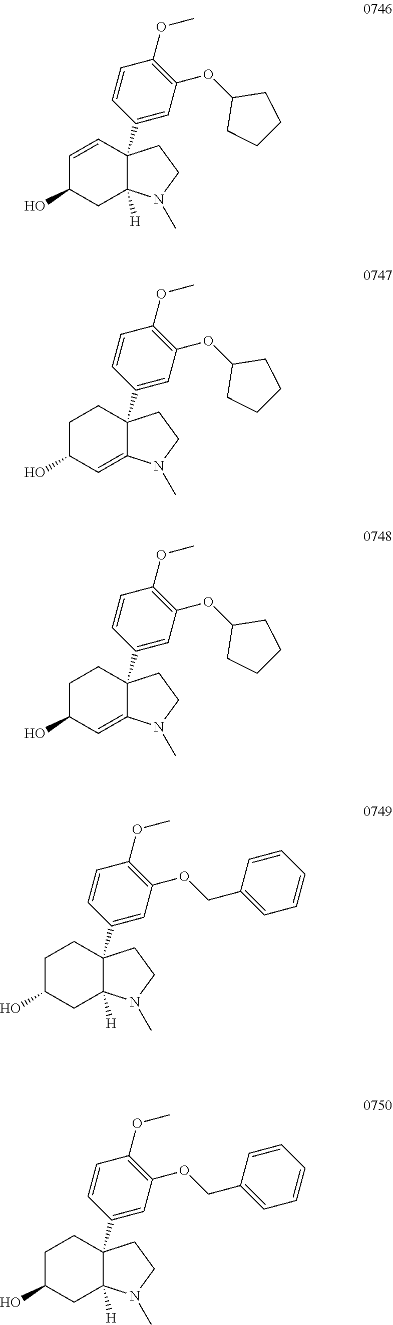 [SMILES: COc1ccc([C@@]23C=C[C@H](O)C[C@@H]2N(C)CC3)cc1OC1CCCC1 ; COc1ccc([C@@]23CC[C@@H](O)C=C2N(C)CC3)cc1OC1CCCC1 ; COc1ccc([C@@]23CC[C@H](O)C=C2N(C)CC3)cc1OC1CCCC1 ; COc1ccc([C@@]23CC[C@@H](O)C[C@@H]2N(C)CC3)cc1OCc1ccccc1 ; COc1ccc([C@@]23CC[C@H](O)C[C@@H]2N(C)CC3)cc1OCc1ccccc1]

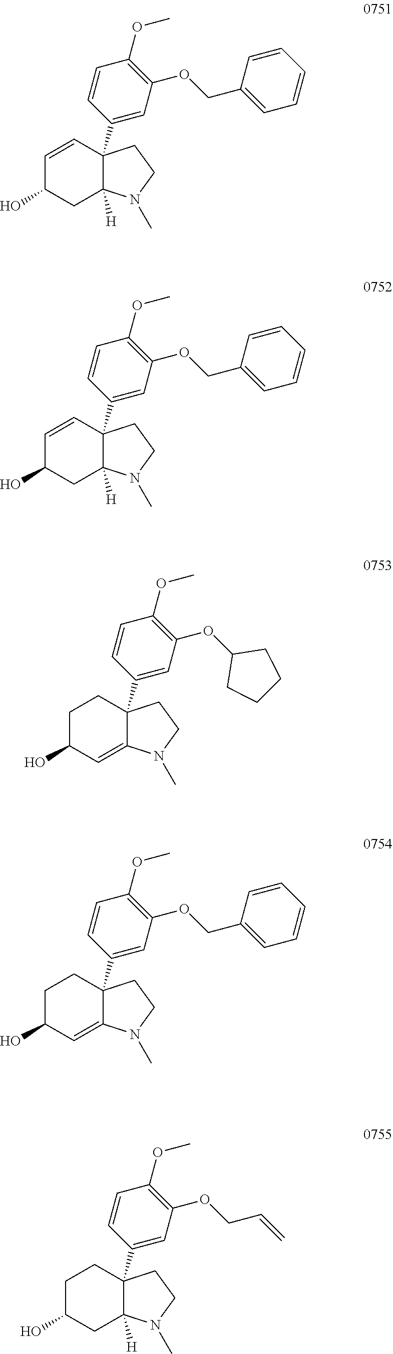 [SMILES: COc1ccc([C@@]23C=C[C@@H](O)C[C@@H]2N(C)CC3)cc1OCc1ccccc1 ; COc1ccc([C@@]23C=C[C@H](O)C[C@@H]2N(C)CC3)cc1OCc1ccccc1 ; COc1ccc([C@@]23CC[C@H](O)C=C2N(C)CC3)cc1OC1CCCC1 ; COc1ccc([C@@]23CC[C@H](O)C=C2N(C)CC3)cc1OCc1ccccc1 ; C=CCOc1cc([C@@]23CC[C@@H](O)C[C@@H]2N(C)CC3)ccc1OC]

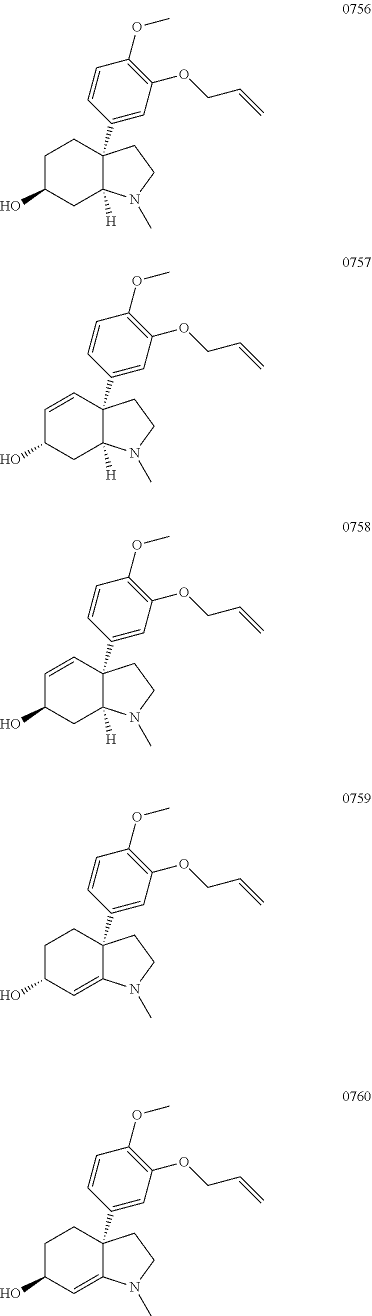 [SMILES: C=CCOc1cc([C@@]23CC[C@H](O)C[C@@H]2N(C)CC3)ccc1OC ; C=CCOc1cc([C@@]23C=C[C@@H](O)C[C@@H]2N(C)CC3)ccc1OC ; C=CCOc1cc([C@@]23C=C[C@H](O)C[C@@H]2N(C)CC3)ccc1OC ; C=CCOc1cc([C@@]23CC[C@@H](O)C=C2N(C)CC3)ccc1OC ; C=CCOc1cc([C@@]23CC[C@H](O)C=C2N(C)CC3)ccc1OC]

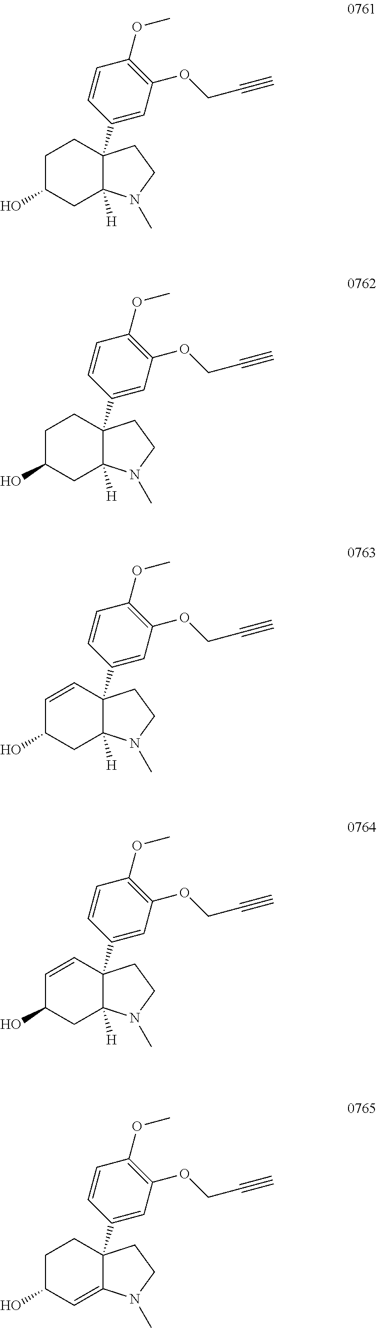 [SMILES: C#CCOc1cc([C@@]23CC[C@@H](O)C[C@@H]2N(C)CC3)ccc1OC ; C#CCOc1cc([C@@]23CC[C@H](O)C[C@@H]2N(C)CC3)ccc1OC ; C#CCOc1cc([C@@]23C=C[C@@H](O)C[C@@H]2N(C)CC3)ccc1OC ; C#CCOc1cc([C@@]23C=C[C@H](O)C[C@@H]2N(C)CC3)ccc1OC ; C#CCOc1cc([C@@]23CC[C@@H](O)C=C2N(C)CC3)ccc1OC]

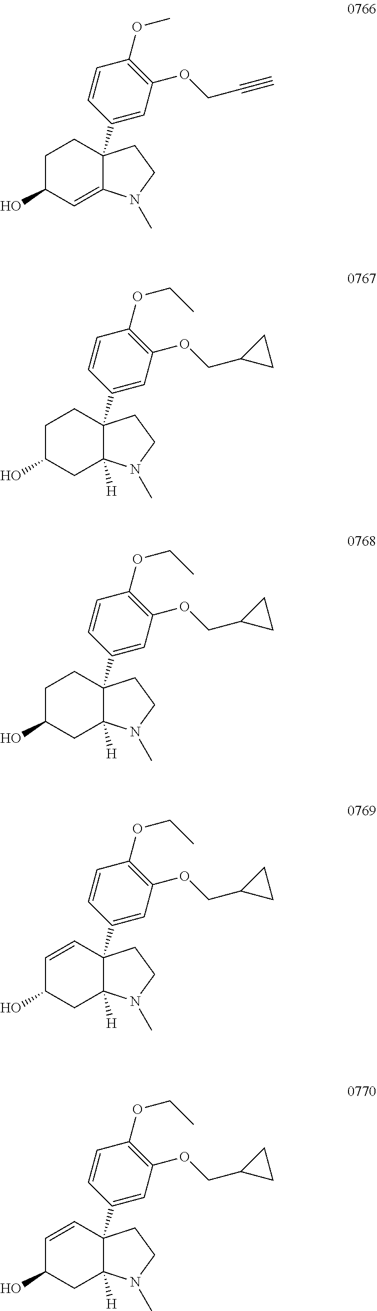 [SMILES: C#CCOc1cc([C@@]23CC[C@H](O)C=C2N(C)CC3)ccc1OC ; CCOc1ccc([C@@]23CC[C@@H](O)C[C@@H]2N(C)CC3)cc1OCC1CC1 ; CCOc1ccc([C@@]23CC[C@H](O)C[C@@H]2N(C)CC3)cc1OCC1CC1 ; CCOc1ccc([C@@]23C=C[C@@H](O)C[C@@H]2N(C)CC3)cc1OCC1CC1 ; CCOc1ccc([C@@]23C=C[C@H](O)C[C@@H]2N(C)CC3)cc1OCC1CC1]

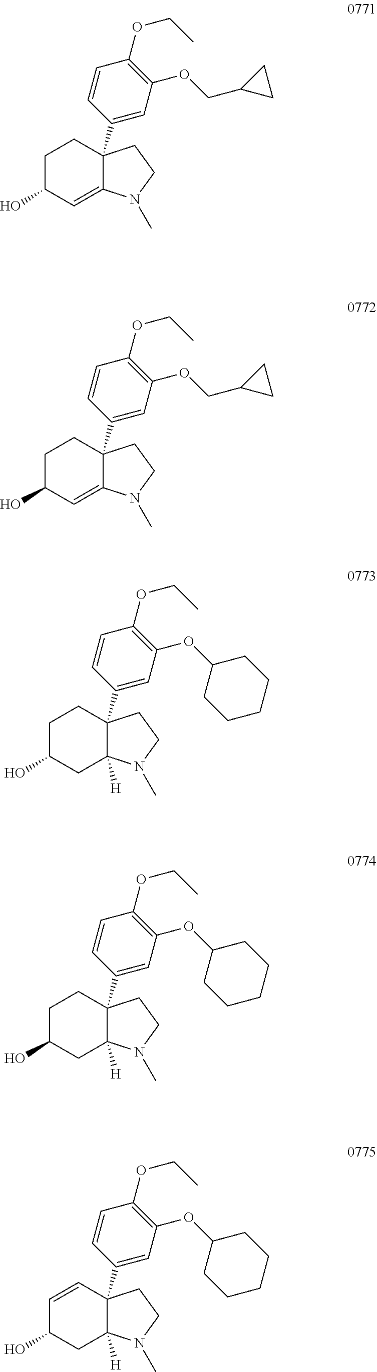 [SMILES: CCOc1ccc([C@@]23CC[C@@H](O)C=C2N(C)CC3)cc1OCC1CC1 ; CCOc1ccc([C@@]23CC[C@H](O)C=C2N(C)CC3)cc1OCC1CC1 ; CCOc1ccc([C@@]23CC[C@@H](O)C[C@@H]2N(C)CC3)cc1OC1CCCCC1 ; CCOc1ccc([C@@]23CC[C@H](O)C[C@@H]2N(C)CC3)cc1OC1CCCCC1 ; CCOc1ccc([C@@]23C=C[C@@H](O)C[C@@H]2N(C)CC3)cc1OC1CCCCC1]

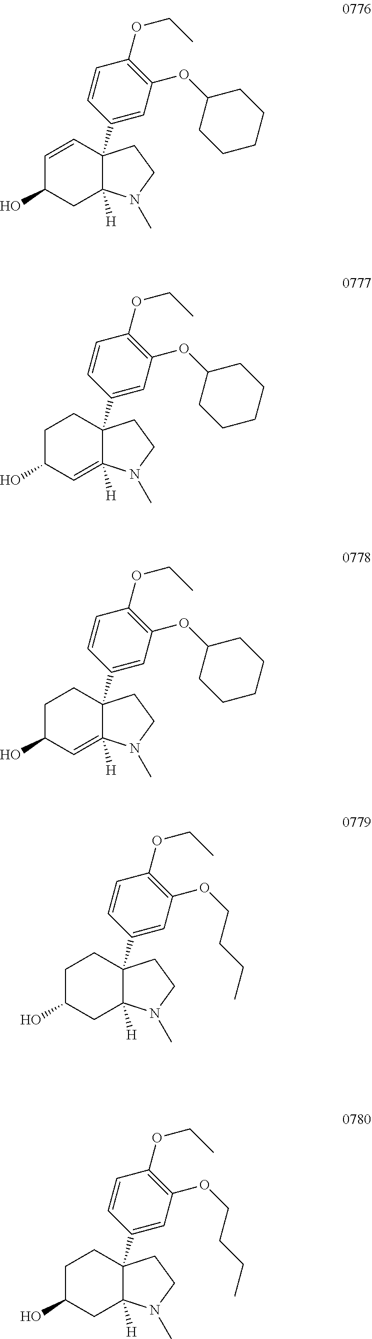

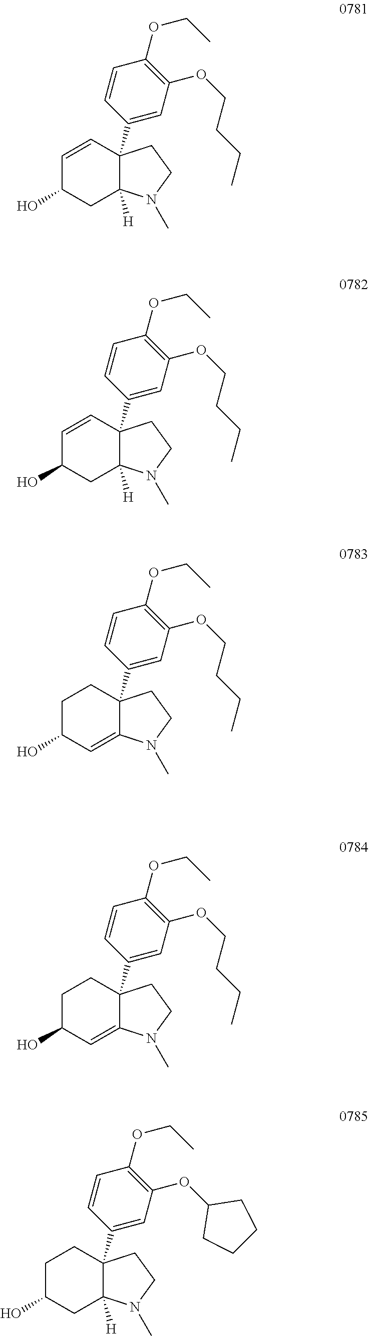 [SMILES: CCCCOc1cc([C@@]23C=C[C@@H](O)C[C@@H]2N(C)CC3)ccc1OCC ; CCCCOc1cc([C@@]23C=C[C@H](O)C[C@@H]2N(C)CC3)ccc1OCC ; CCCCOc1cc([C@@]23CC[C@@H](O)C=C2N(C)CC3)ccc1OCC ; CCCCOc1cc([C@@]23CC[C@H](O)C=C2N(C)CC3)ccc1OCC ; CCOc1ccc([C@@]23CC[C@@H](O)C[C@@H]2N(C)CC3)cc1OC1CCCC1]

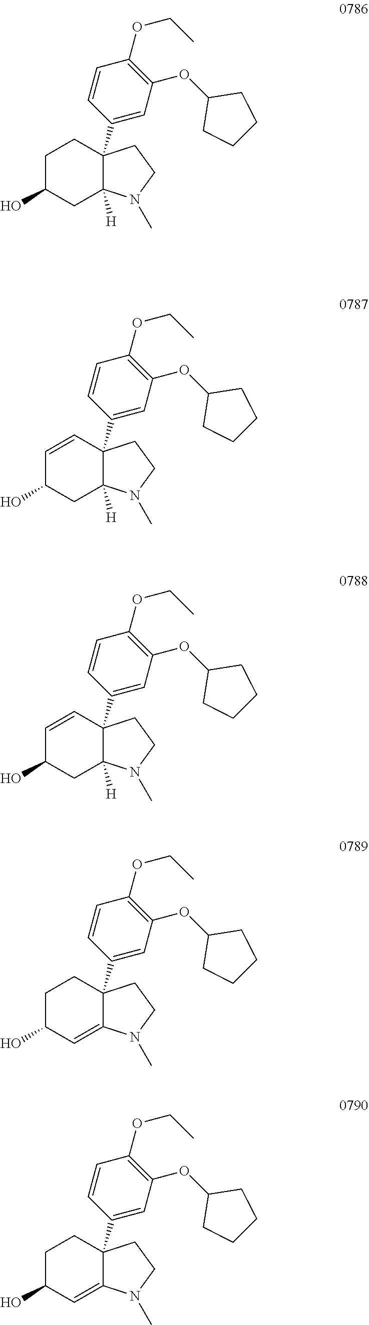 [SMILES: CCOc1ccc([C@@]23CC[C@H](O)C[C@@H]2N(C)CC3)cc1OC1CCCC1 ; CCOc1ccc([C@@]23C=C[C@@H](O)C[C@@H]2N(C)CC3)cc1OC1CCCC1 ; CCOc1ccc([C@@]23C=C[C@H](O)C[C@@H]2N(C)CC3)cc1OC1CCCC1 ; CCOc1ccc([C@@]23CC[C@@H](O)C=C2N(C)CC3)cc1OC1CCCC1 ; CCOc1ccc([C@@]23CC[C@H](O)C=C2N(C)CC3)cc1OC1CCCC1]

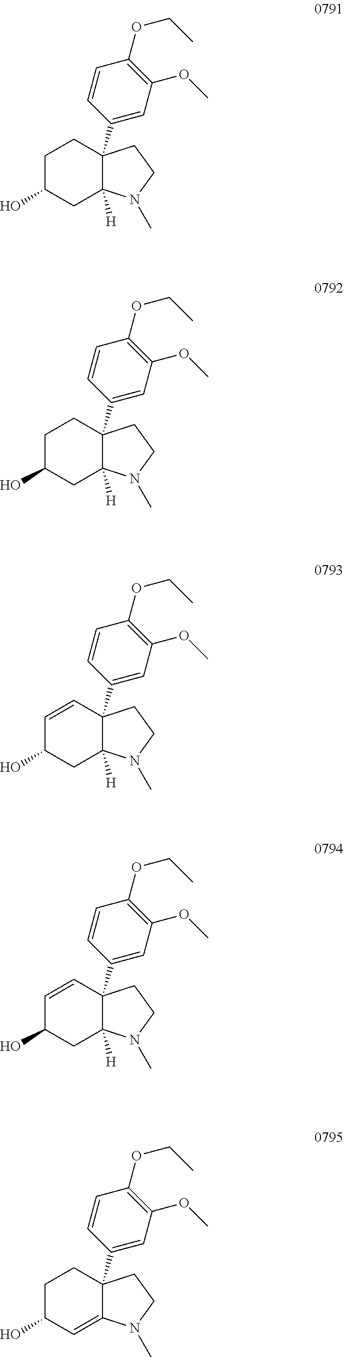 [SMILES: CCOc1ccc([C@@]23CC[C@@H](O)C[C@@H]2N(C)CC3)cc1OC ; CCOc1ccc([C@@]23CC[C@H](O)C[C@@H]2N(C)CC3)cc1OC ; CCOc1ccc([C@@]23C=C[C@@H](O)C[C@@H]2N(C)CC3)cc1OC ; CCOc1ccc([C@@]23C=C[C@H](O)C[C@@H]2N(C)CC3)cc1OC ; CCOc1ccc([C@@]23CC[C@@H](O)C=C2N(C)CC3)cc1OC]

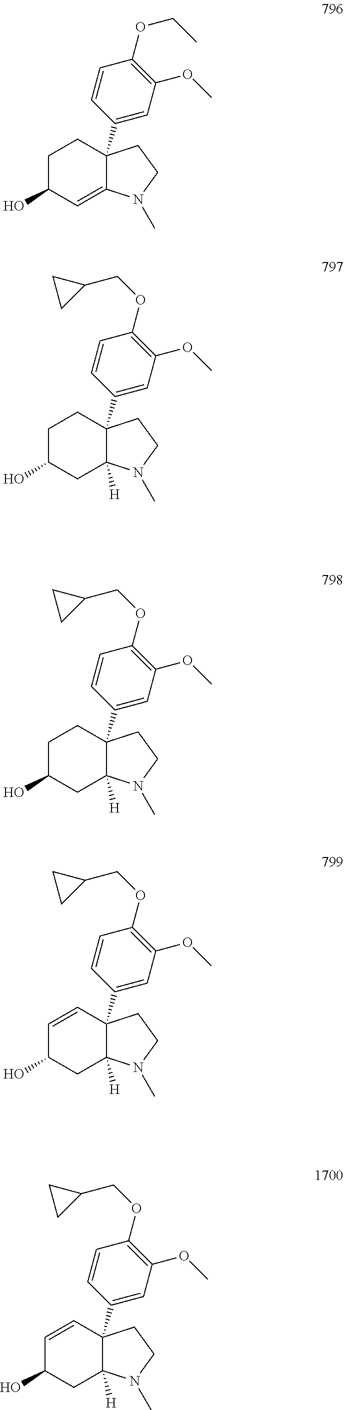 [SMILES: CCOc1ccc([C@@]23CC[C@H](O)C=C2N(C)CC3)cc1OC ; COc1cc([C@@]23CC[C@@H](O)C[C@@H]2N(C)CC3)ccc1OCC1CC1 ; COc1cc([C@@]23CC[C@H](O)C[C@@H]2N(C)CC3)ccc1OCC1CC1 ; COc1cc([C@@]23C=C[C@@H](O)C[C@@H]2N(C)CC3)ccc1OCC1CC1 ; COc1cc([C@@]23C=C[C@H](O)C[C@@H]2N(C)CC3)ccc1OCC1CC1]

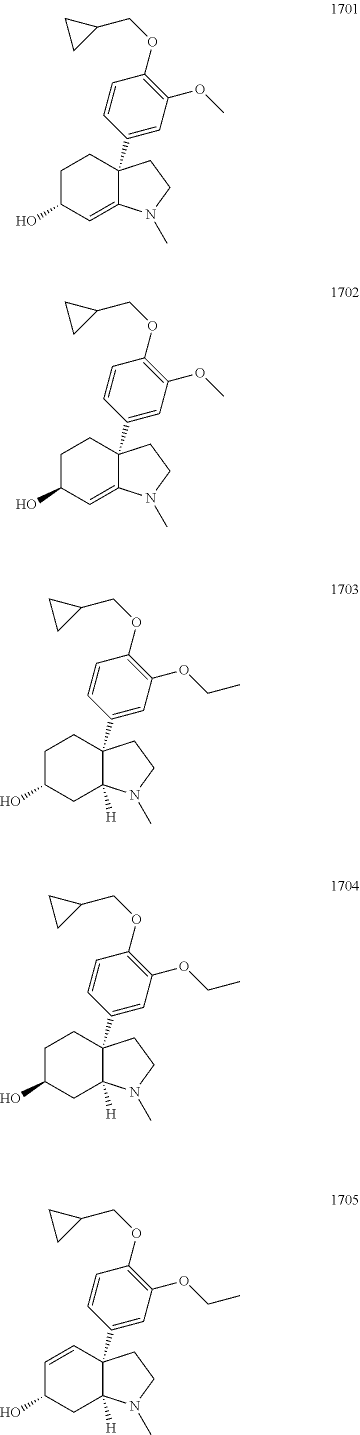 [SMILES: COc1cc([C@@]23CC[C@@H](O)C=C2N(C)CC3)ccc1OCC1CC1 ; COc1cc([C@@]23CC[C@H](O)C=C2N(C)CC3)ccc1OCC1CC1 ; CCOc1cc([C@@]23CC[C@@H](O)C[C@@H]2N(C)CC3)ccc1OCC1CC1 ; CCOc1cc([C@@]23CC[C@H](O)C[C@@H]2N(C)CC3)ccc1OCC1CC1 ; CCOc1cc([C@@]23C=C[C@@H](O)C[C@@H]2N(C)CC3)ccc1OCC1CC1]

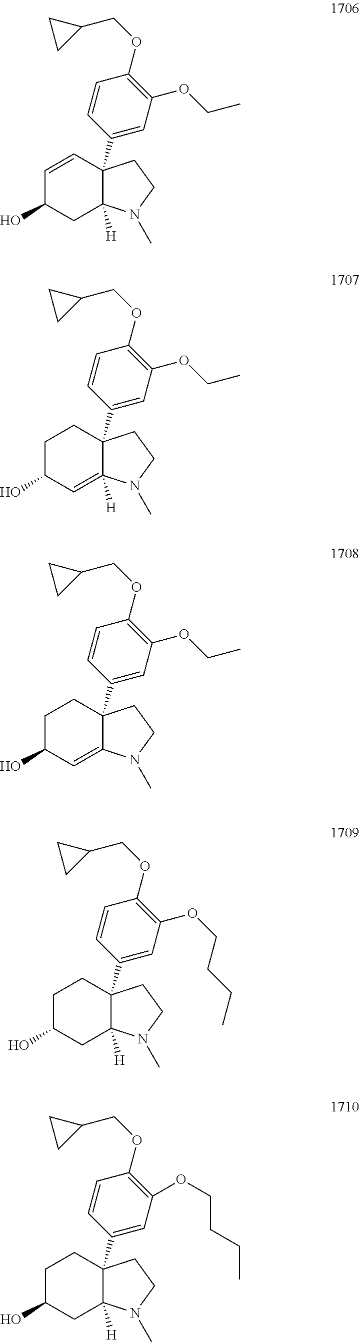

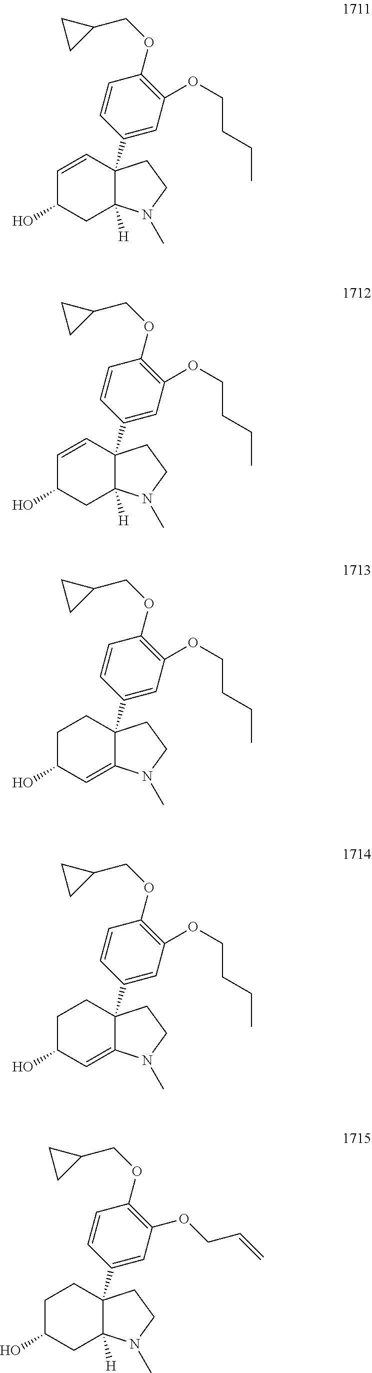 [SMILES: CCCCOc1cc([C@@]23C=C[C@@H](O)C[C@@H]2N(C)CC3)ccc1OCC1CC1 ; CCCCOc1cc([C@@]23C=C[C@@H](O)C[C@@H]2N(C)CC3)ccc1OCC1CC1 ; CCCCOc1cc([C@@]23CC[C@@H](O)C=C2N(C)CC3)ccc1OCC1CC1 ; CCCCOc1cc([C@@]23CC[C@@H](O)C=C2N(C)CC3)ccc1OCC1CC1 ; C=CCOc1cc([C@@]23CC[C@@H](O)C[C@@H]2N(C)CC3)ccc1OCC1CC1]

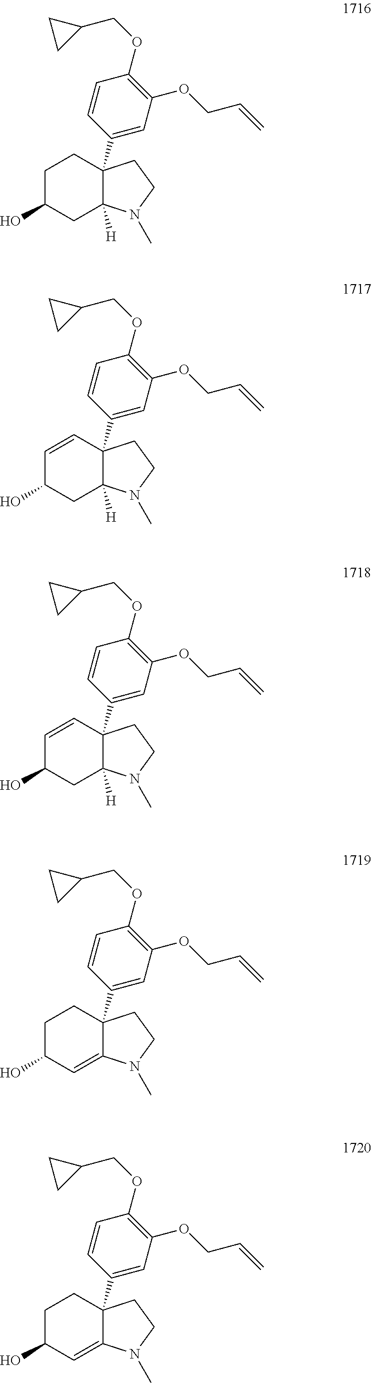 [SMILES: C=CCOc1cc([C@@]23CC[C@H](O)C[C@@H]2N(C)CC3)ccc1OCC1CC1 ; C=CCOc1cc([C@@]23C=C[C@@H](O)C[C@@H]2N(C)CC3)ccc1OCC1CC1 ; C=CCOc1cc([C@@]23C=C[C@H](O)C[C@@H]2N(C)CC3)ccc1OCC1CC1 ; C=CCOc1cc([C@@]23CC[C@@H](O)C=C2N(C)CC3)ccc1OCC1CC1 ; C=CCOc1cc([C@@]23CC[C@H](O)C=C2N(C)CC3)ccc1OCC1CC1]

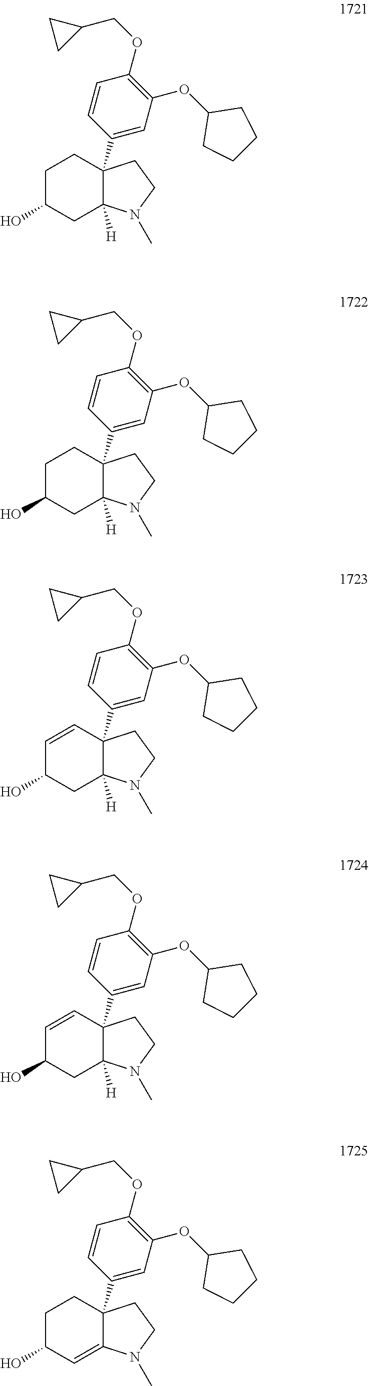 [SMILES: CN1CC[C@]2(c3ccc(OCC4CC4)c(OC4CCCC4)c3)CC[C@@H](O)C[C@H]12 ; CN1CC[C@]2(c3ccc(OCC4CC4)c(OC4CCCC4)c3)CC[C@H](O)C[C@H]12 ; CN1CC[C@]2(c3ccc(OCC4CC4)c(OC4CCCC4)c3)C=C[C@@H](O)C[C@H]12 ; CN1CC[C@]2(c3ccc(OCC4CC4)c(OC4CCCC4)c3)C=C[C@H](O)C[C@H]12 ; CN1CC[C@]2(c3ccc(OCC4CC4)c(OC4CCCC4)c3)CC[C@@H](O)C=C12]

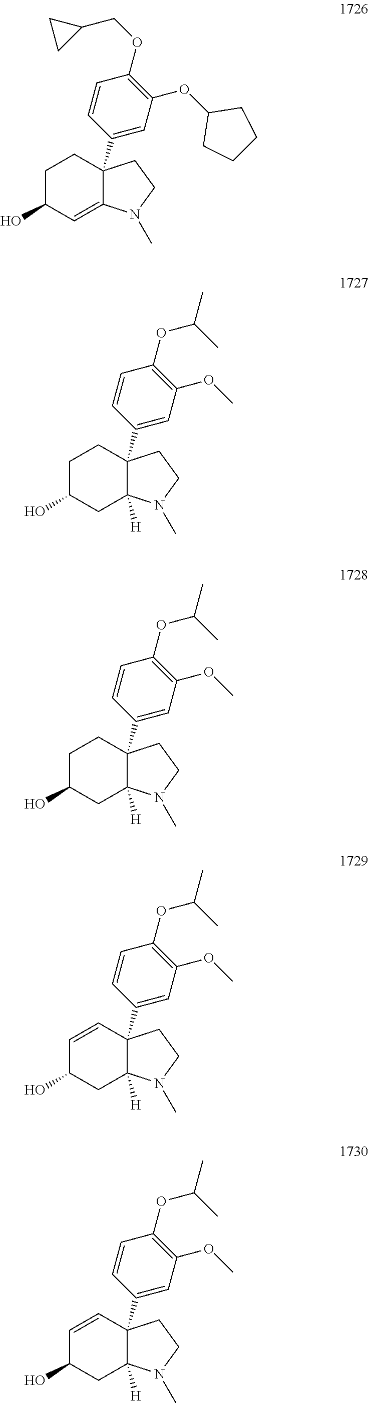 [SMILES: CN1CC[C@]2(c3ccc(OCC4CC4)c(OC4CCCC4)c3)CC[C@H](O)C=C12 ; COc1cc([C@@]23CC[C@@H](O)C[C@@H]2N(C)CC3)ccc1OC(C)C ; COc1cc([C@@]23CC[C@H](O)C[C@@H]2N(C)CC3)ccc1OC(C)C ; COc1cc([C@@]23C=C[C@@H](O)C[C@@H]2N(C)CC3)ccc1OC(C)C ; COc1cc([C@@]23C=C[C@H](O)C[C@@H]2N(C)CC3)ccc1OC(C)C]

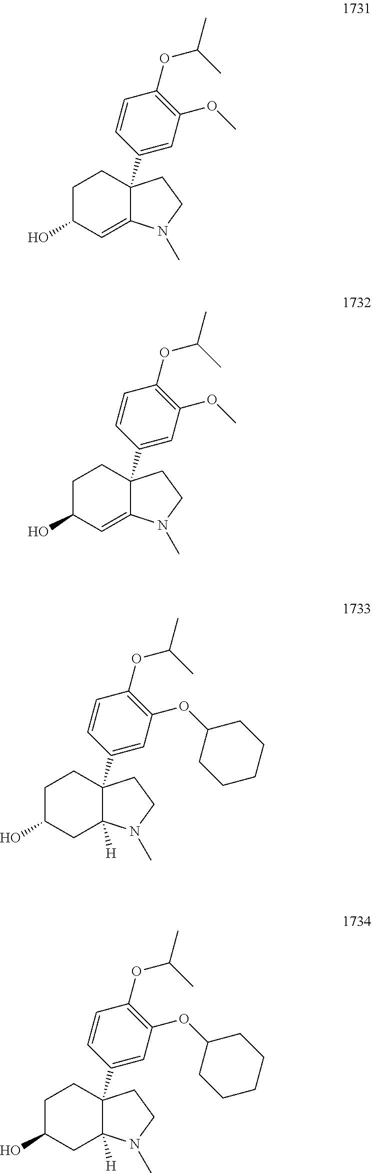 [SMILES: COc1cc([C@@]23CC[C@@H](O)C=C2N(C)CC3)ccc1OC(C)C ; COc1cc([C@@]23CC[C@H](O)C=C2N(C)CC3)ccc1OC(C)C ; CC(C)Oc1ccc([C@@]23CC[C@@H](O)C[C@@H]2N(C)CC3)cc1OC1CCCCC1 ; CC(C)Oc1ccc([C@@]23CC[C@H](O)C[C@@H]2N(C)CC3)cc1OC1CCCCC1]

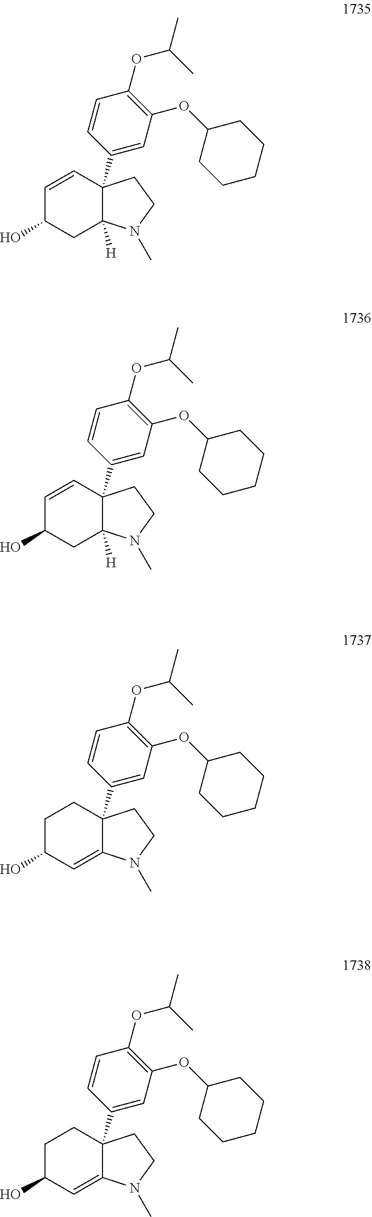 [SMILES: CC(C)Oc1ccc([C@@]23C=C[C@@H](O)C[C@@H]2N(C)CC3)cc1OC1CCCCC1 ; CC(C)Oc1ccc([C@@]23C=C[C@H](O)C[C@@H]2N(C)CC3)cc1OC1CCCCC1 ; CC(C)Oc1ccc([C@@]23CC[C@@H](O)C=C2N(C)CC3)cc1OC1CCCCC1 ; CC(C)Oc1ccc([C@@]23CC[C@H](O)C=C2N(C)CC3)cc1OC1CCCCC1]

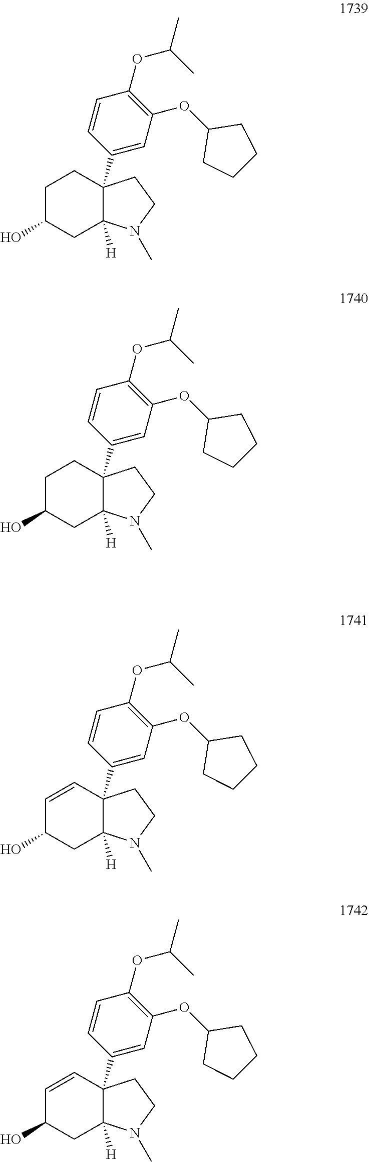 [SMILES: CC(C)Oc1ccc([C@@]23CC[C@@H](O)C[C@@H]2N(C)CC3)cc1OC1CCCC1 ; CC(C)Oc1ccc([C@@]23CC[C@H](O)C[C@@H]2N(C)CC3)cc1OC1CCCC1 ; CC(C)Oc1ccc([C@@]23C=C[C@@H](O)C[C@@H]2N(C)CC3)cc1OC1CCCC1 ; CC(C)Oc1ccc([C@@]23C=C[C@H](O)C[C@@H]2N(C)CC3)cc1OC1CCCC1]

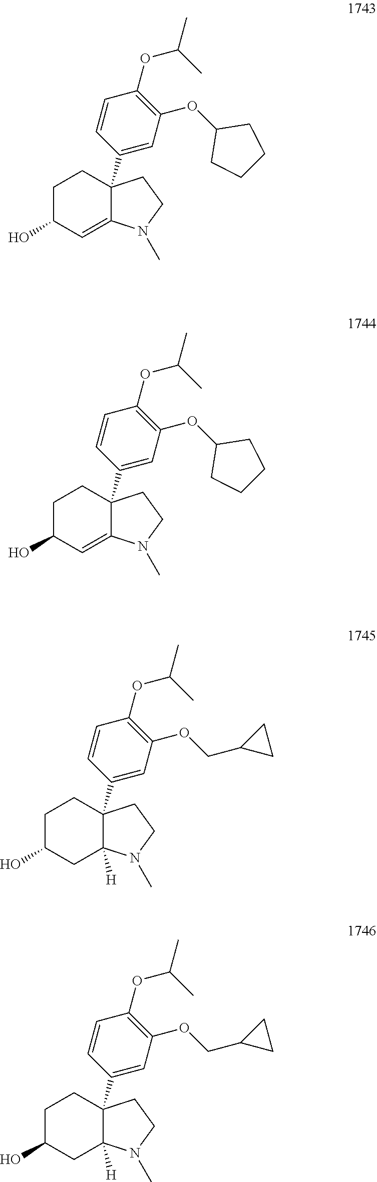 [SMILES: CC(C)Oc1ccc([C@@]23CC[C@@H](O)C=C2N(C)CC3)cc1OC1CCCC1 ; CC(C)Oc1ccc([C@@]23CC[C@H](O)C=C2N(C)CC3)cc1OC1CCCC1 ; CC(C)Oc1ccc([C@@]23CC[C@@H](O)C[C@@H]2N(C)CC3)cc1OCC1CC1 ; CC(C)Oc1ccc([C@@]23CC[C@H](O)C[C@@H]2N(C)CC3)cc1OCC1CC1]

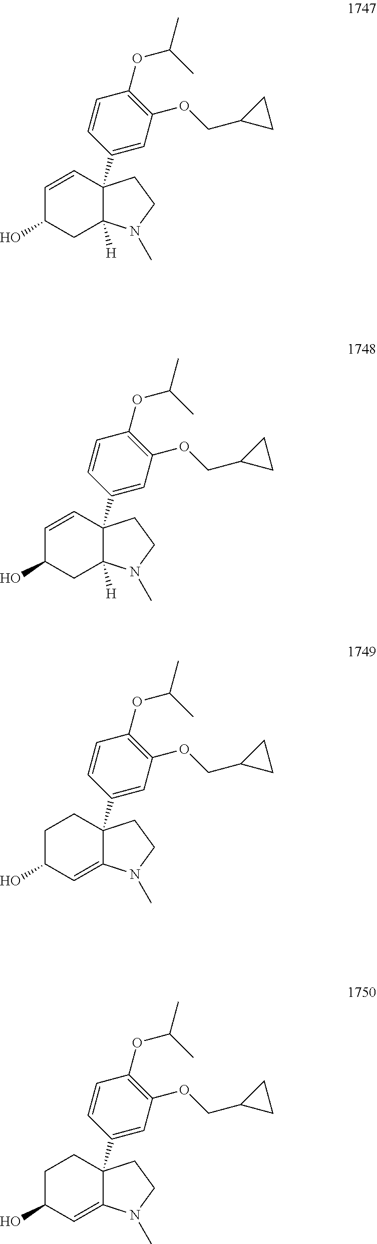 [SMILES: CC(C)Oc1ccc([C@@]23C=C[C@@H](O)C[C@@H]2N(C)CC3)cc1OCC1CC1 ; CC(C)Oc1ccc([C@@]23C=C[C@H](O)C[C@@H]2N(C)CC3)cc1OCC1CC1 ; CC(C)Oc1ccc([C@@]23CC[C@@H](O)C=C2N(C)CC3)cc1OCC1CC1 ; CC(C)Oc1ccc([C@@]23CC[C@H](O)C=C2N(C)CC3)cc1OCC1CC1]

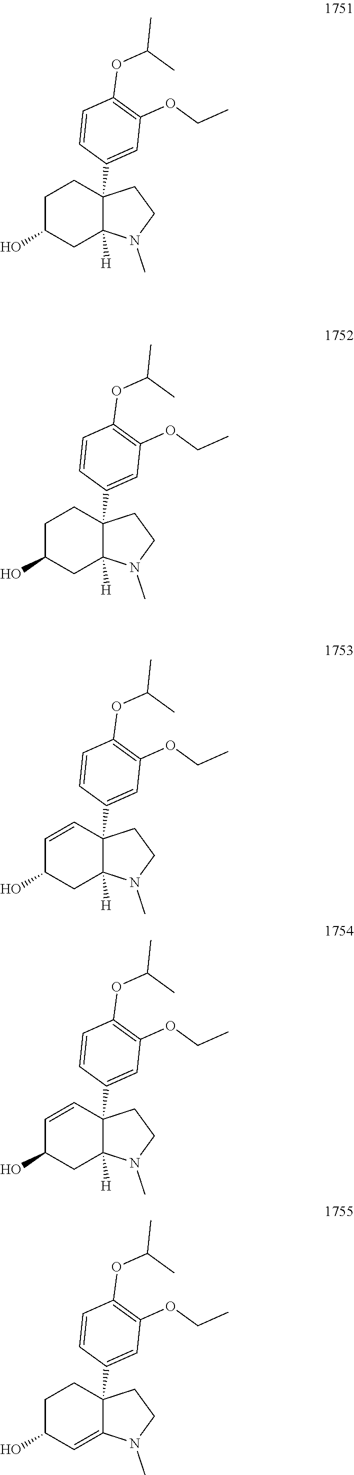

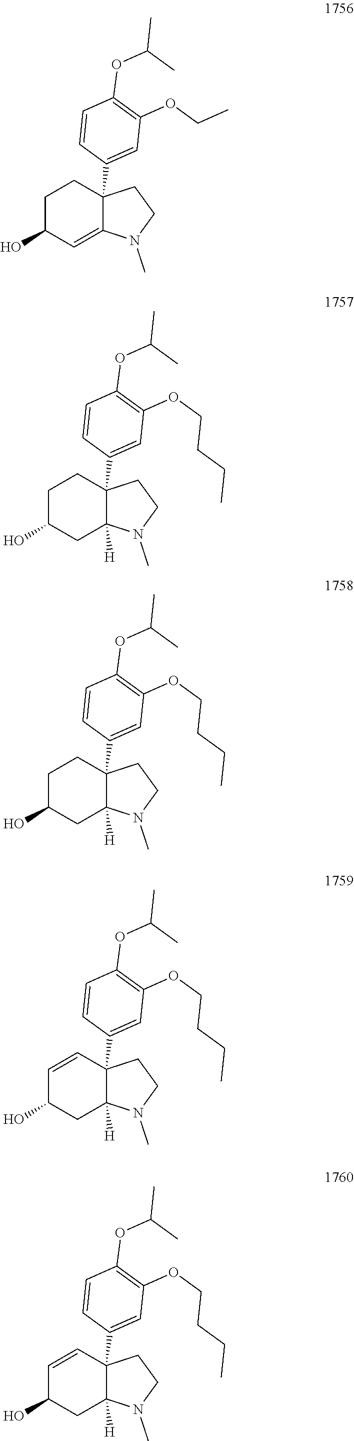

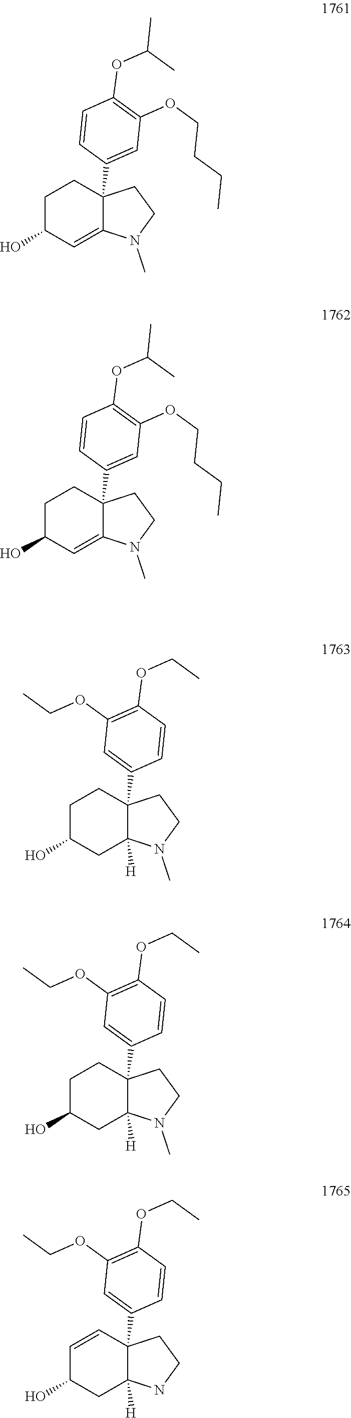

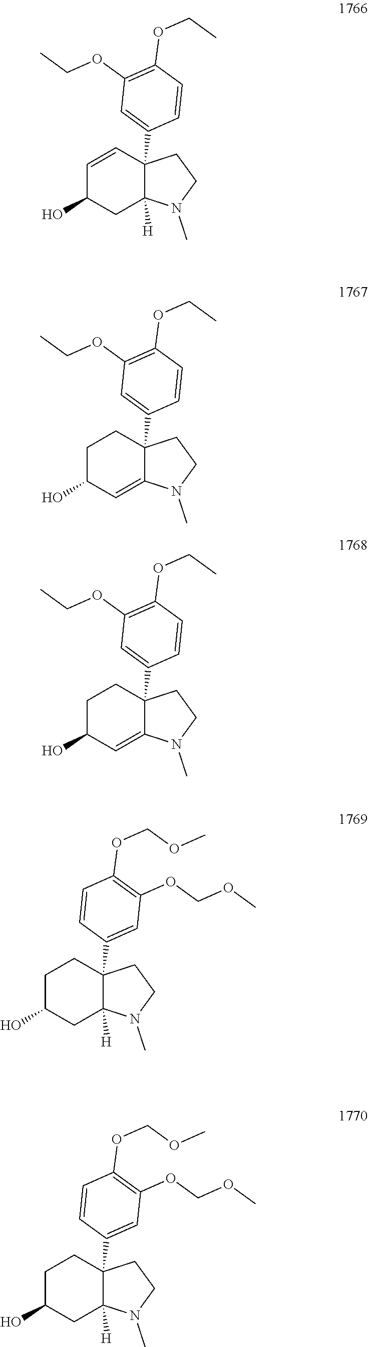

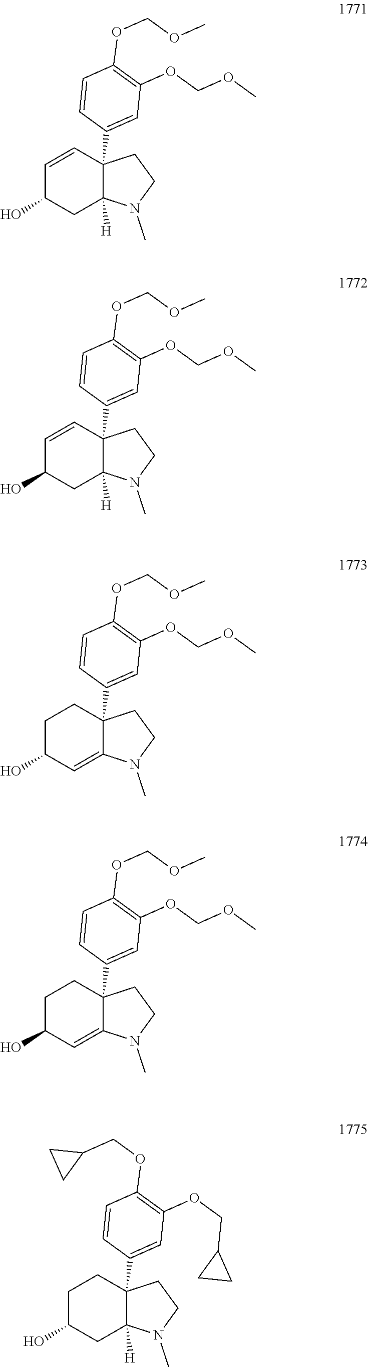 [SMILES: COCOc1ccc([C@@]23C=C[C@@H](O)C[C@@H]2N(C)CC3)cc1OCOC ; COCOc1ccc([C@@]23C=C[C@H](O)C[C@@H]2N(C)CC3)cc1OCOC ; COCOc1ccc([C@@]23CC[C@@H](O)C=C2N(C)CC3)cc1OCOC ; COCOc1ccc([C@@]23CC[C@H](O)C=C2N(C)CC3)cc1OCOC ; CN1CC[C@]2(c3ccc(OCC4CC4)c(OCC4CC4)c3)CC[C@@H](O)C[C@H]12]

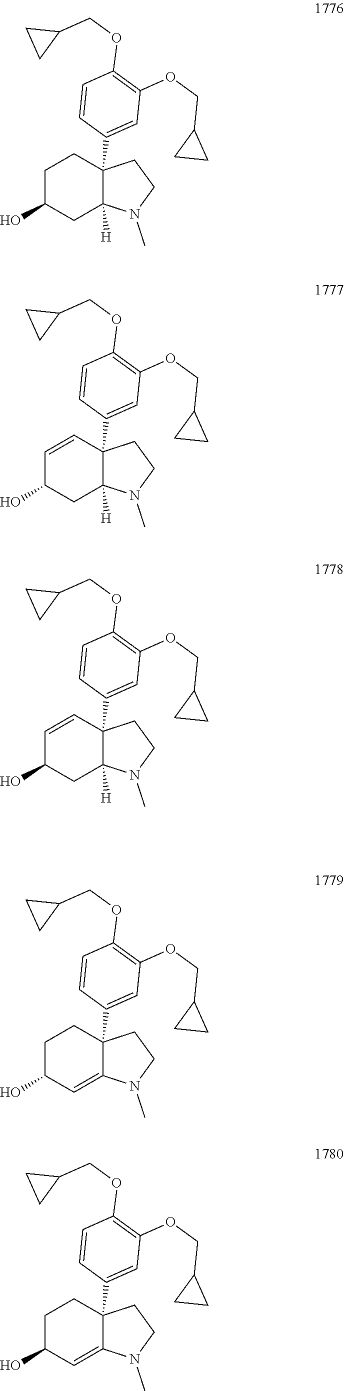 [SMILES: CN1CC[C@]2(c3ccc(OCC4CC4)c(OCC4CC4)c3)CC[C@H](O)C[C@H]12 ; CN1CC[C@]2(c3ccc(OCC4CC4)c(OCC4CC4)c3)C=C[C@@H](O)C[C@H]12 ; CN1CC[C@]2(c3ccc(OCC4CC4)c(OCC4CC4)c3)C=C[C@H](O)C[C@H]12 ; CN1CC[C@]2(c3ccc(OCC4CC4)c(OCC4CC4)c3)CC[C@@H](O)C=C12 ; CN1CC[C@]2(c3ccc(OCC4CC4)c(OCC4CC4)c3)CC[C@H](O)C=C12]

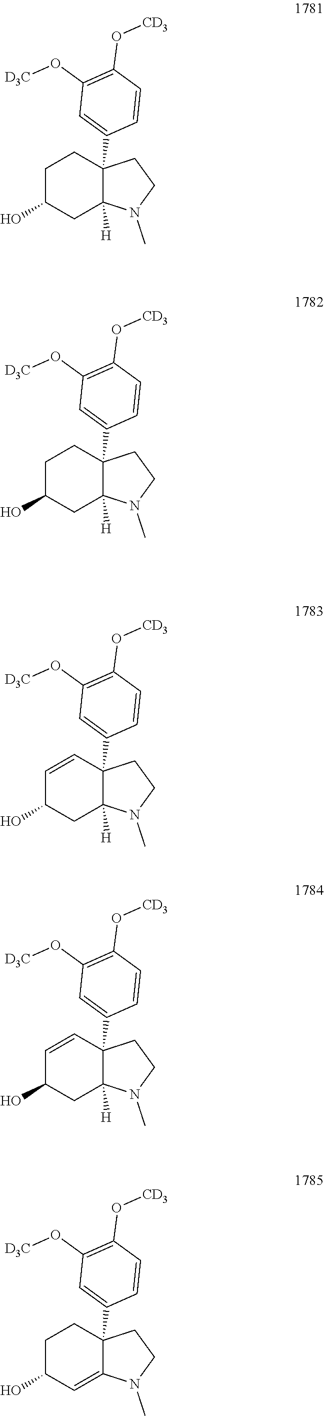 [SMILES: [2H]C([2H])([2H])Oc1ccc([C@@]23CC[C@@H](O)C[C@@H]2N(C)CC3)cc1OC([2H])([2H])[2H] ; [2H]C([2H])([2H])Oc1ccc([C@@]23CC[C@H](O)C[C@@H]2N(C)CC3)cc1OC([2H])([2H])[2H] ; [2H]C([2H])([2H])Oc1ccc([C@@]23C=C[C@@H](O)C[C@@H]2N(C)CC3)cc1OC([2H])([2H])[2H] ; [2H]C([2H])([2H])Oc1ccc([C@@]23C=C[C@H](O)C[C@@H]2N(C)CC3)cc1OC([2H])([2H])[2H] ; [2H]C([2H])([2H])Oc1ccc([C@@]23CC[C@@H](O)C=C2N(C)CC3)cc1OC([2H])([2H])[2H]]

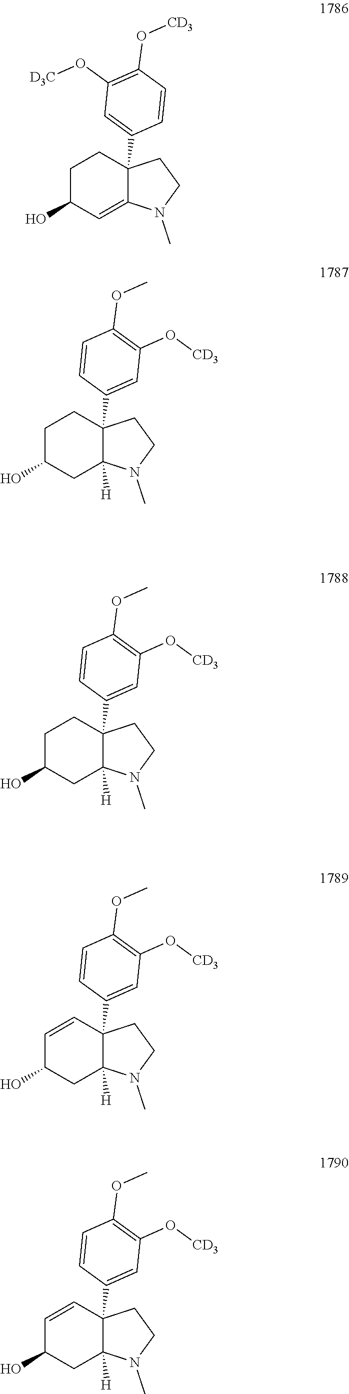

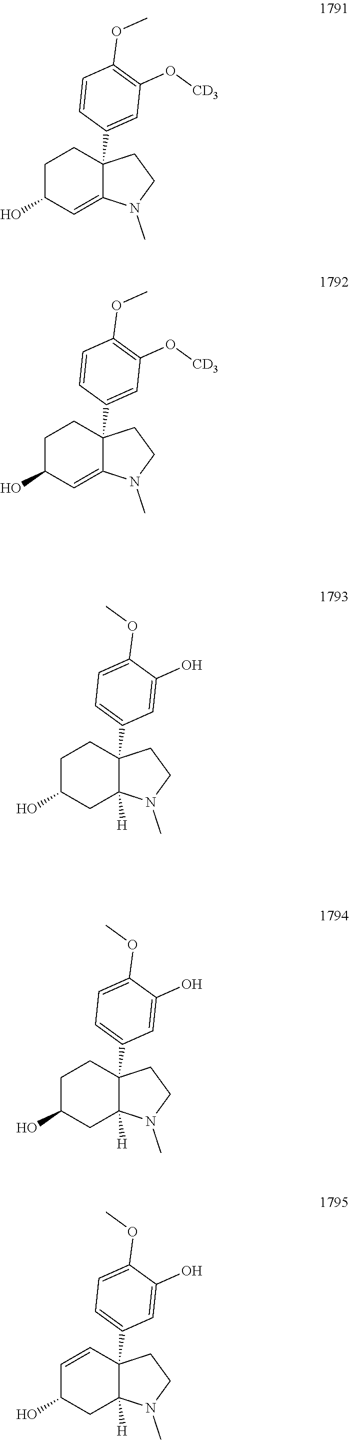 [SMILES: [2H]C([2H])([2H])Oc1cc([C@@]23CC[C@@H](O)C=C2N(C)CC3)ccc1OC ; [2H]C([2H])([2H])Oc1cc([C@@]23CC[C@H](O)C=C2N(C)CC3)ccc1OC ; COc1ccc([C@@]23CC[C@@H](O)C[C@@H]2N(C)CC3)cc1O ; COc1ccc([C@@]23CC[C@H](O)C[C@@H]2N(C)CC3)cc1O ; COc1ccc([C@@]23C=C[C@@H](O)C[C@@H]2N(C)CC3)cc1O]

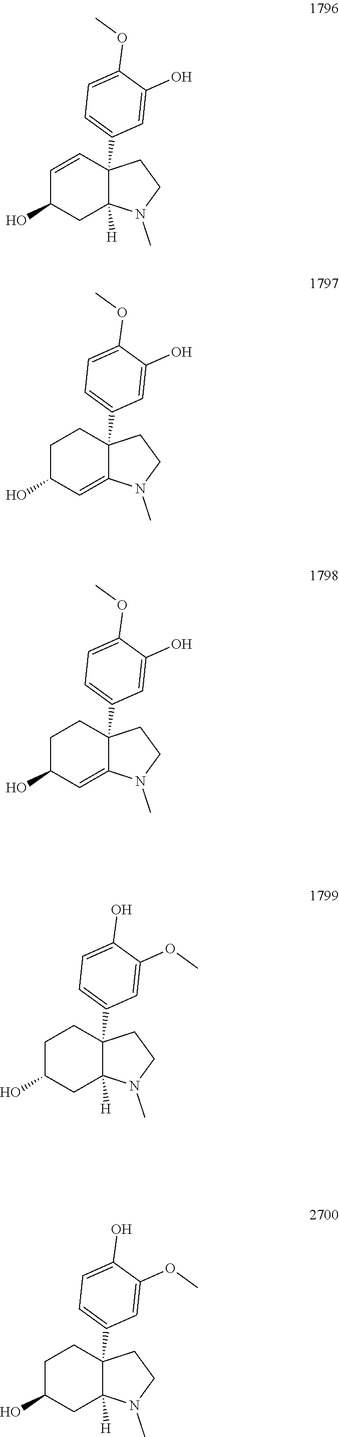 [SMILES: COc1ccc([C@@]23C=C[C@H](O)C[C@@H]2N(C)CC3)cc1O ; COc1ccc([C@@]23CC[C@@H](O)C=C2N(C)CC3)cc1O ; COc1ccc([C@@]23CC[C@H](O)C=C2N(C)CC3)cc1O ; COc1cc([C@@]23CC[C@@H](O)C[C@@H]2N(C)CC3)ccc1O ; COc1cc([C@@]23CC[C@H](O)C[C@@H]2N(C)CC3)ccc1O]

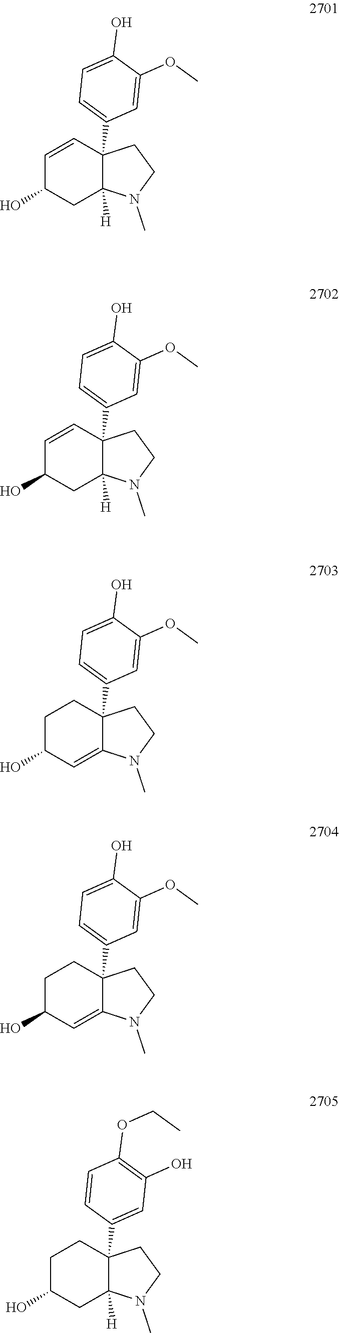 [SMILES: COc1cc([C@@]23C=C[C@@H](O)C[C@@H]2N(C)CC3)ccc1O ; COc1cc([C@@]23C=C[C@H](O)C[C@@H]2N(C)CC3)ccc1O ; COc1cc([C@@]23CC[C@@H](O)C=C2N(C)CC3)ccc1O ; COc1cc([C@@]23CC[C@H](O)C=C2N(C)CC3)ccc1O ; CCOc1ccc([C@@]23CC[C@@H](O)C[C@@H]2N(C)CC3)cc1O]

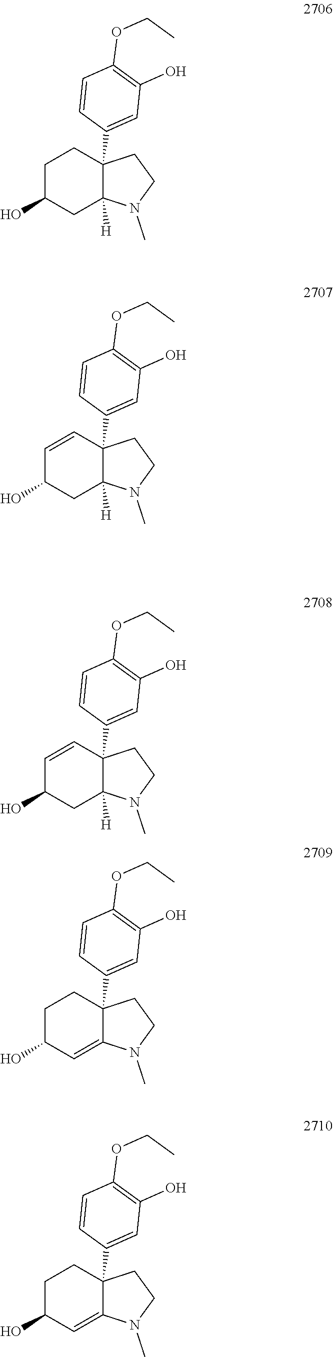

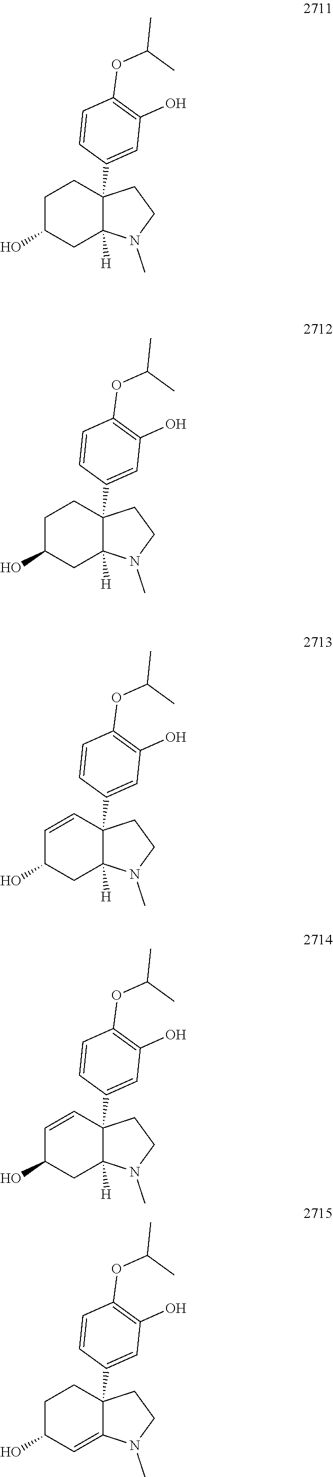

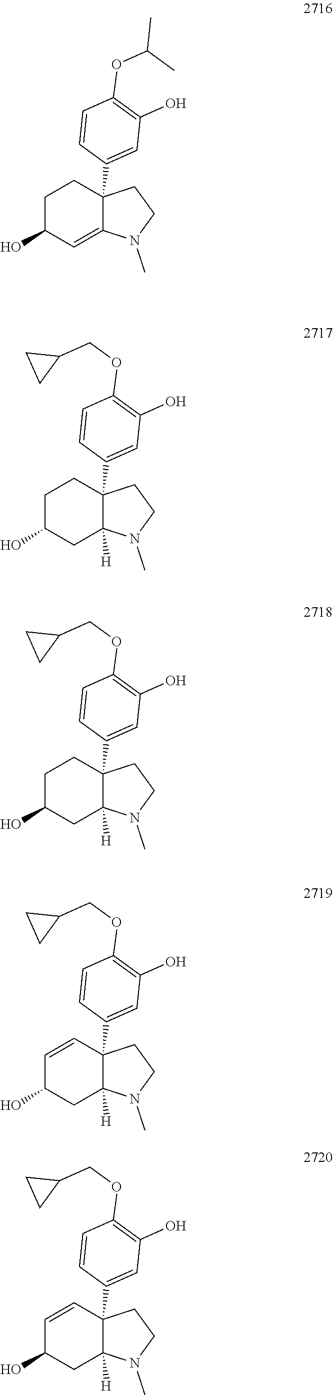 [SMILES: CC(C)Oc1ccc([C@@]23CC[C@H](O)C=C2N(C)CC3)cc1O ; CN1CC[C@]2(c3ccc(OCC4CC4)c(O)c3)CC[C@@H](O)C[C@H]12 ; CN1CC[C@]2(c3ccc(OCC4CC4)c(O)c3)CC[C@H](O)C[C@H]12 ; CN1CC[C@]2(c3ccc(OCC4CC4)c(O)c3)C=C[C@@H](O)C[C@H]12 ; CN1CC[C@]2(c3ccc(OCC4CC4)c(O)c3)C=C[C@H](O)C[C@H]12]

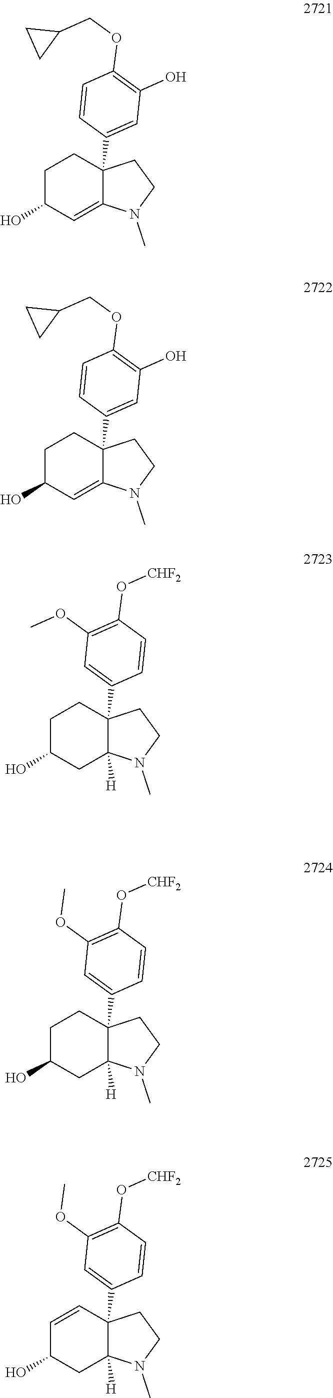 [SMILES: CN1CC[C@]2(c3ccc(OCC4CC4)c(O)c3)CC[C@@H](O)C=C12 ; CN1CC[C@]2(c3ccc(OCC4CC4)c(O)c3)CC[C@H](O)C=C12 ; COc1cc([C@@]23CC[C@@H](O)C[C@@H]2N(C)CC3)ccc1OC(F)F ; COc1cc([C@@]23CC[C@H](O)C[C@@H]2N(C)CC3)ccc1OC(F)F ; COc1cc([C@@]23C=C[C@@H](O)C[C@@H]2N(C)CC3)ccc1OC(F)F]

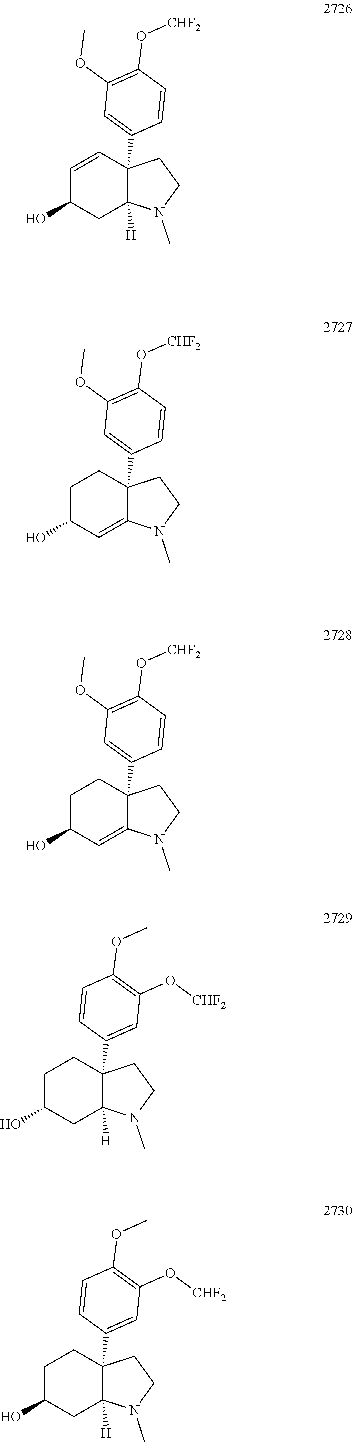 [SMILES: COc1cc([C@@]23C=C[C@H](O)C[C@@H]2N(C)CC3)ccc1OC(F)F ; COc1cc([C@@]23CC[C@@H](O)C=C2N(C)CC3)ccc1OC(F)F ; COc1cc([C@@]23CC[C@H](O)C=C2N(C)CC3)ccc1OC(F)F ; COc1ccc([C@@]23CC[C@@H](O)C[C@@H]2N(C)CC3)cc1OC(F)F ; COc1ccc([C@@]23CC[C@H](O)C[C@@H]2N(C)CC3)cc1OC(F)F]

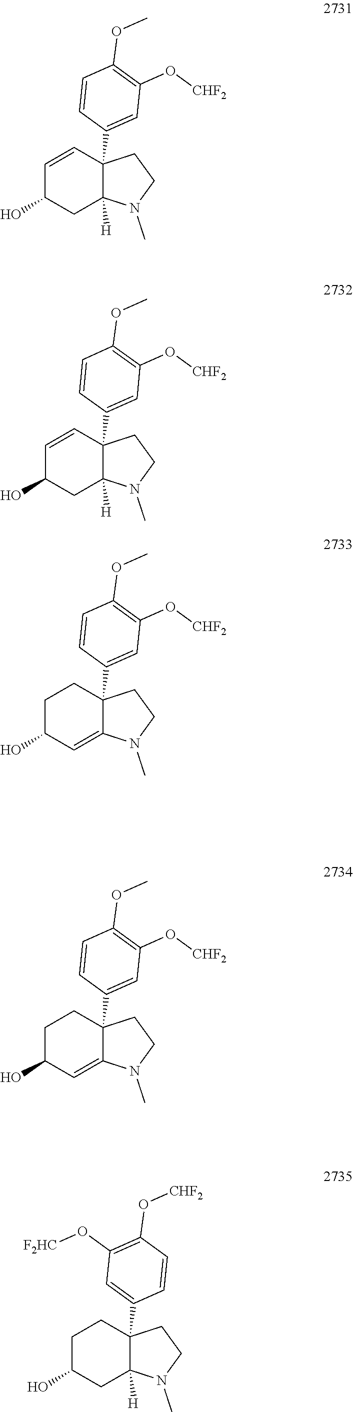

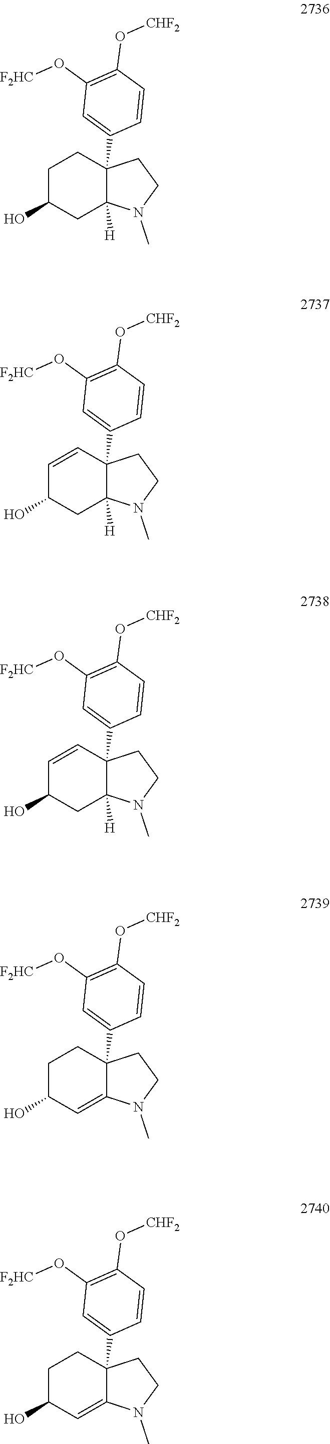 [SMILES: CN1CC[C@]2(c3ccc(OC(F)F)c(OC(F)F)c3)CC[C@H](O)C[C@H]12 ; CN1CC[C@]2(c3ccc(OC(F)F)c(OC(F)F)c3)C=C[C@@H](O)C[C@H]12 ; CN1CC[C@]2(c3ccc(OC(F)F)c(OC(F)F)c3)C=C[C@H](O)C[C@H]12 ; CN1CC[C@]2(c3ccc(OC(F)F)c(OC(F)F)c3)CC[C@@H](O)C=C12 ; CN1CC[C@]2(c3ccc(OC(F)F)c(OC(F)F)c3)CC[C@H](O)C=C12]

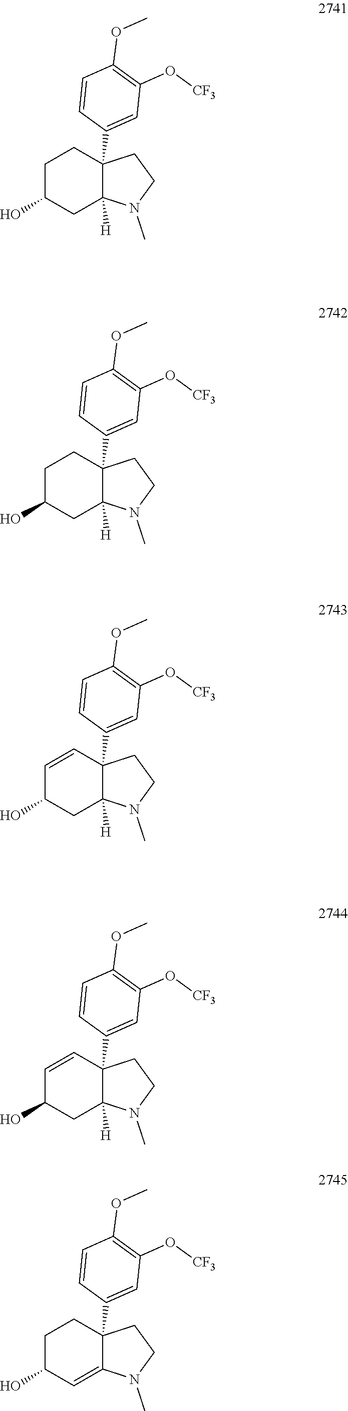

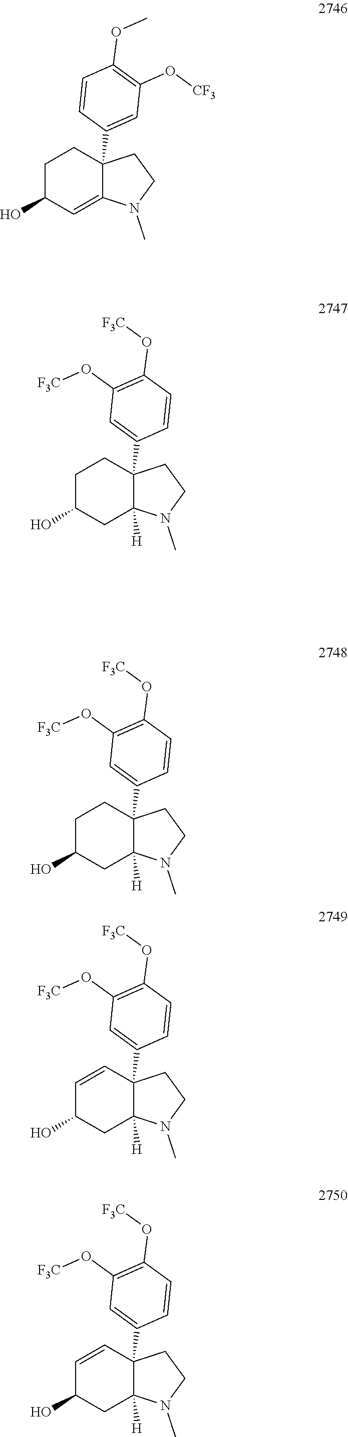 [SMILES: COc1ccc([C@@]23CC[C@H](O)C=C2N(C)CC3)cc1OC(F)(F)F ; CN1CC[C@]2(c3ccc(OC(F)(F)F)c(OC(F)(F)F)c3)CC[C@@H](O)C[C@H]12 ; CN1CC[C@]2(c3ccc(OC(F)(F)F)c(OC(F)(F)F)c3)CC[C@H](O)C[C@H]12 ; CN1CC[C@]2(c3ccc(OC(F)(F)F)c(OC(F)(F)F)c3)C=C[C@@H](O)C[C@H]12 ; CN1CC[C@]2(c3ccc(OC(F)(F)F)c(OC(F)(F)F)c3)C=C[C@H](O)C[C@H]12]

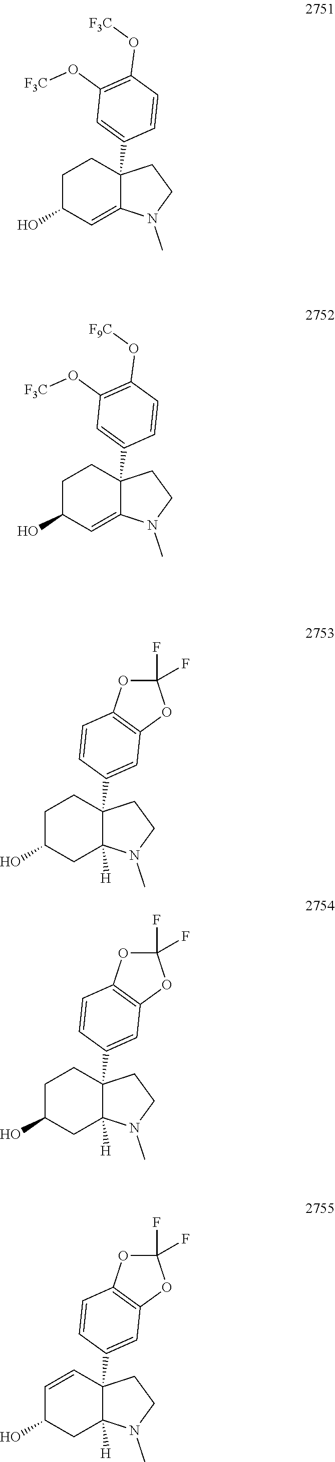

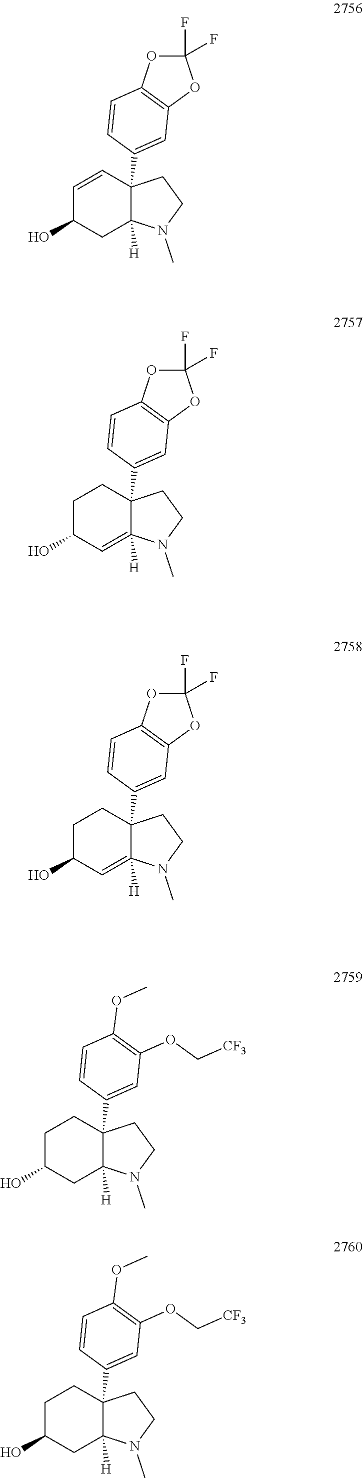

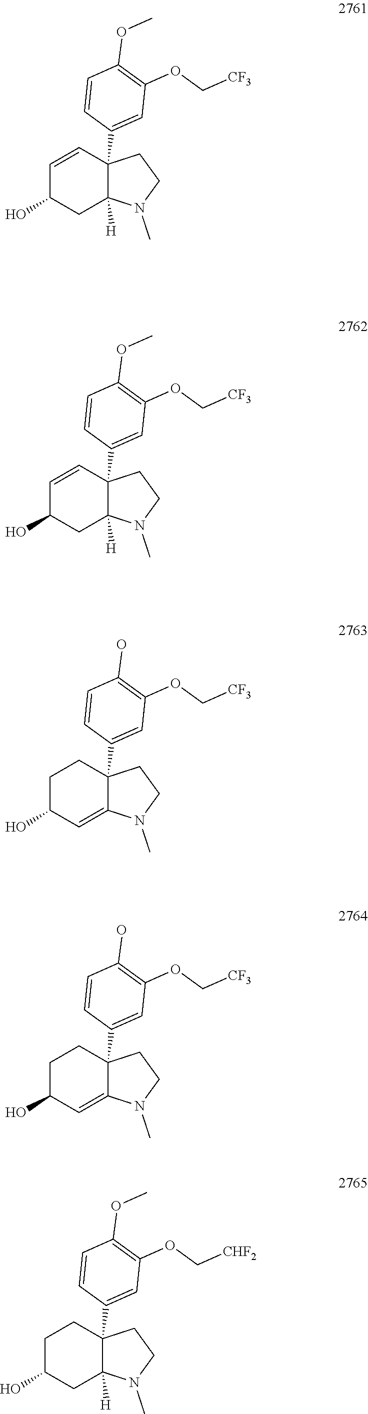

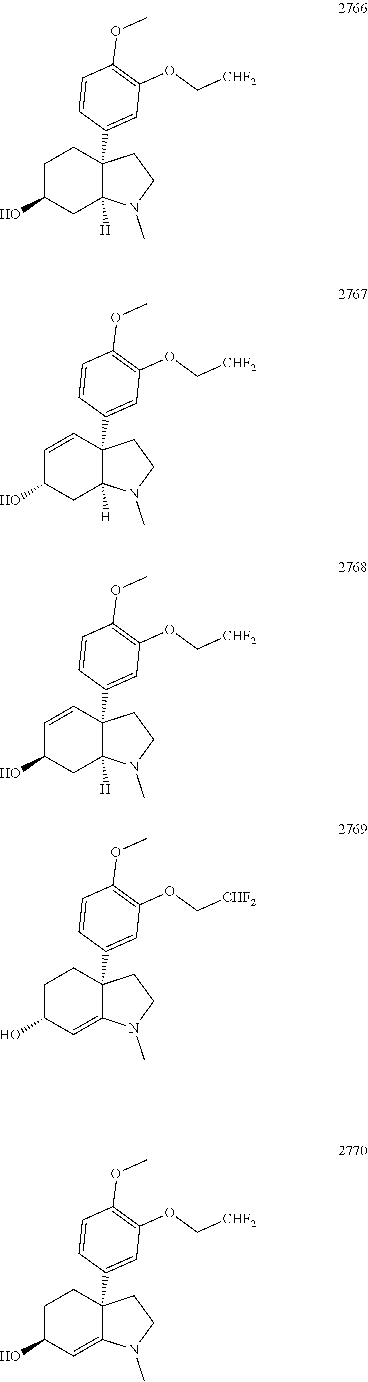

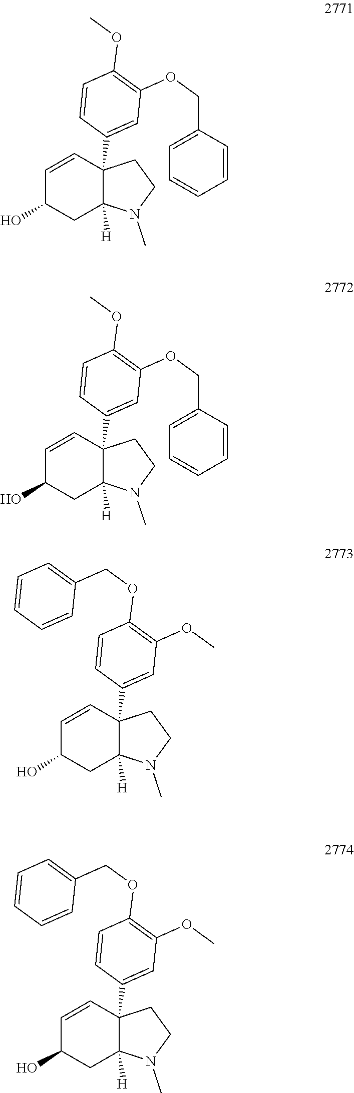 [SMILES: COc1ccc([C@@]23C=C[C@@H](O)C[C@@H]2N(C)CC3)cc1OCc1ccccc1 ; COc1ccc([C@@]23C=C[C@H](O)C[C@@H]2N(C)CC3)cc1OCc1ccccc1 ; COc1cc([C@@]23C=C[C@@H](O)C[C@@H]2N(C)CC3)ccc1OCc1ccccc1 ; COc1cc([C@@]23C=C[C@H](O)C[C@@H]2N(C)CC3)ccc1OCc1ccccc1]

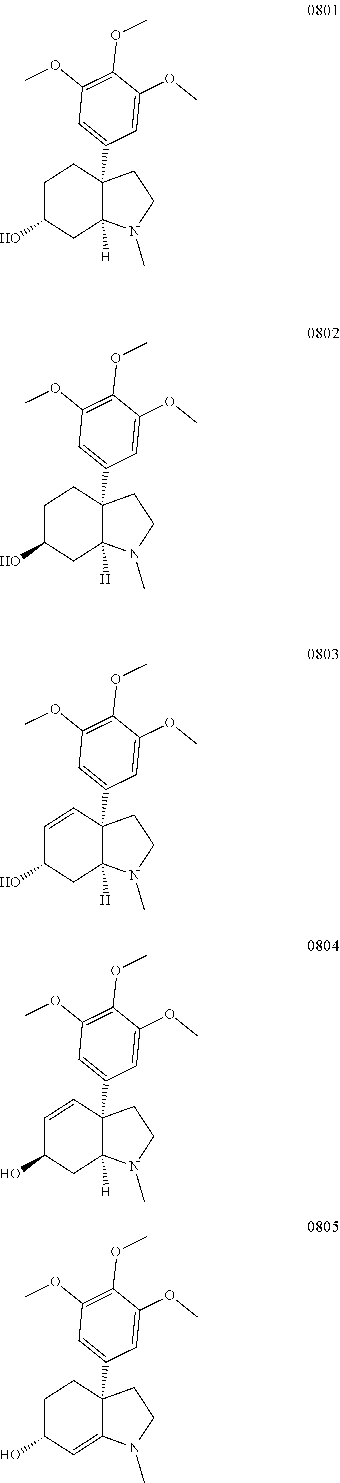

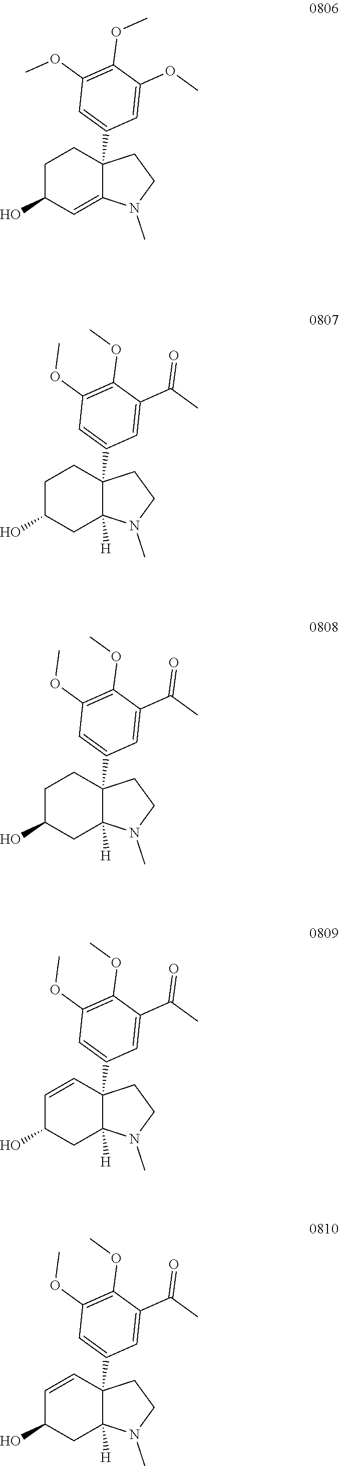 [SMILES: COc1cc([C@@]23CC[C@H](O)C=C2N(C)CC3)cc(OC)c1OC ; COc1cc([C@@]23CC[C@@H](O)C[C@@H]2N(C)CC3)cc(C(C)=O)c1OC ; COc1cc([C@@]23CC[C@H](O)C[C@@H]2N(C)CC3)cc(C(C)=O)c1OC ; COc1cc([C@@]23C=C[C@@H](O)C[C@@H]2N(C)CC3)cc(C(C)=O)c1OC ; COc1cc([C@@]23C=C[C@H](O)C[C@@H]2N(C)CC3)cc(C(C)=O)c1OC]

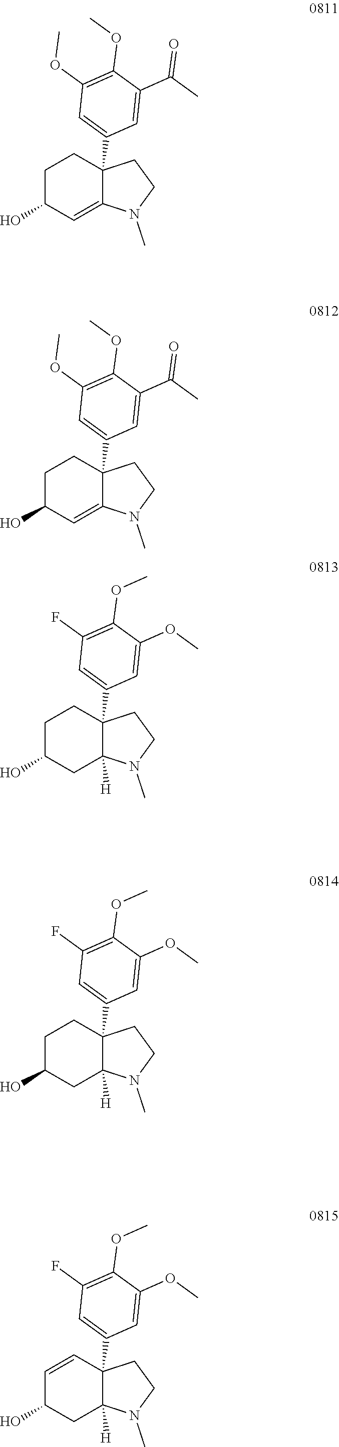

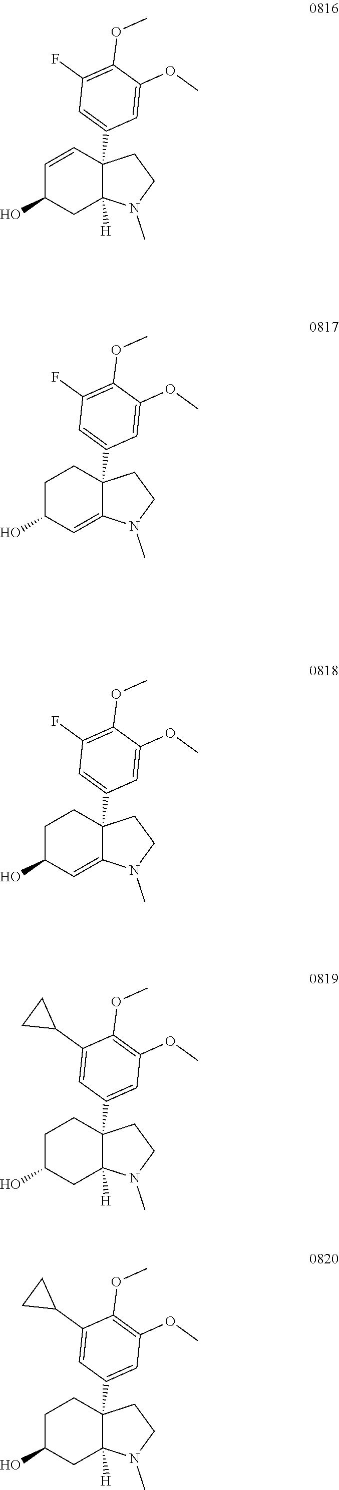 [SMILES: COc1cc([C@@]23C=C[C@H](O)C[C@@H]2N(C)CC3)cc(F)c1OC ; COc1cc([C@@]23CC[C@@H](O)C=C2N(C)CC3)cc(F)c1OC ; COc1cc([C@@]23CC[C@H](O)C=C2N(C)CC3)cc(F)c1OC ; COc1cc([C@@]23CC[C@@H](O)C[C@@H]2N(C)CC3)cc(C2CC2)c1OC ; COc1cc([C@@]23CC[C@H](O)C[C@@H]2N(C)CC3)cc(C2CC2)c1OC]

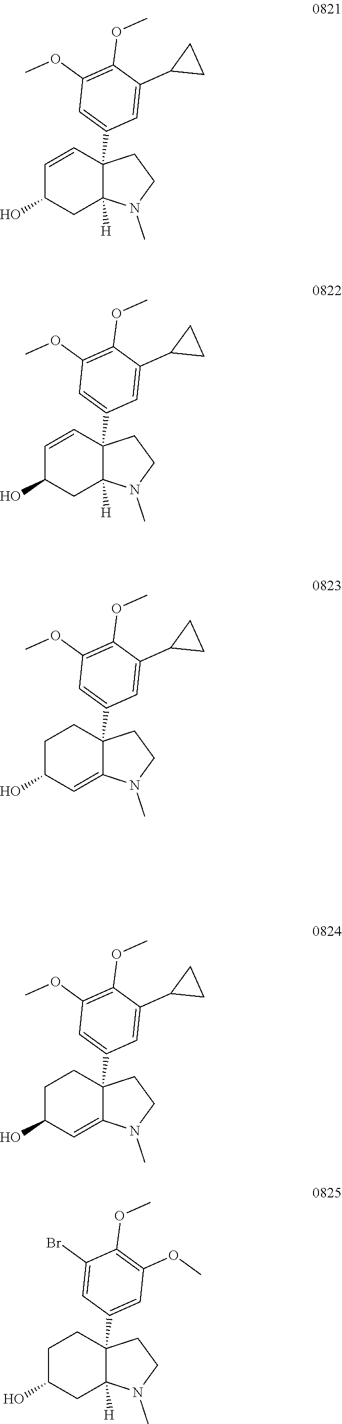

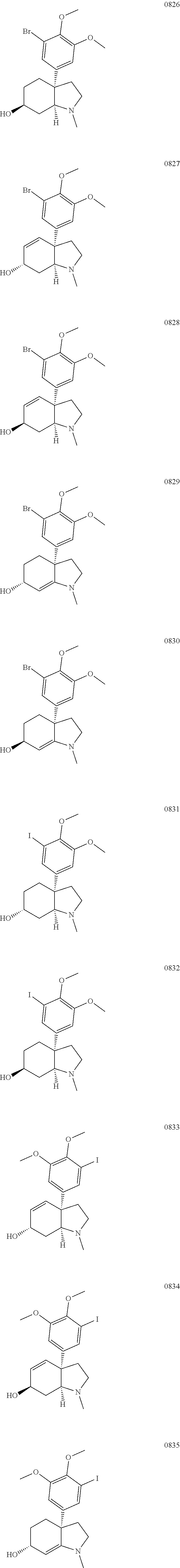

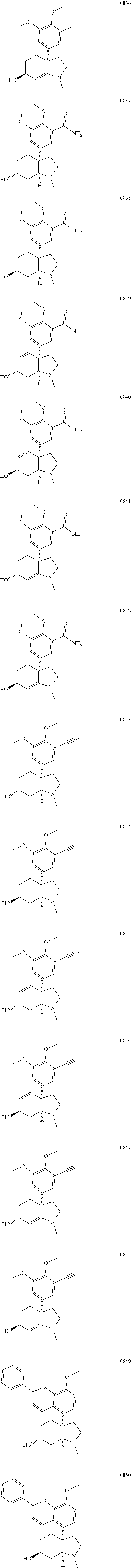

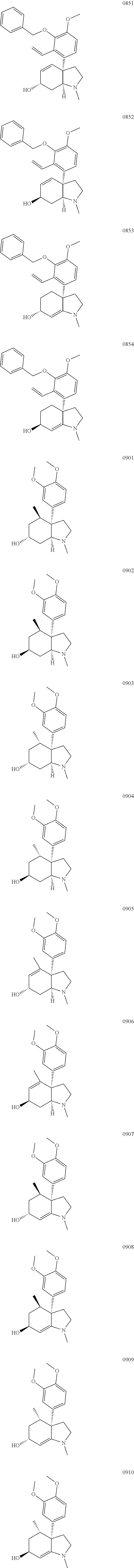

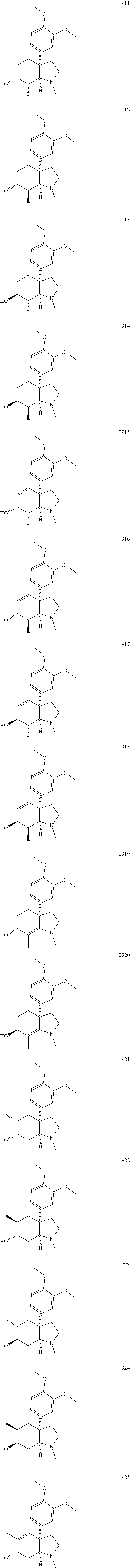

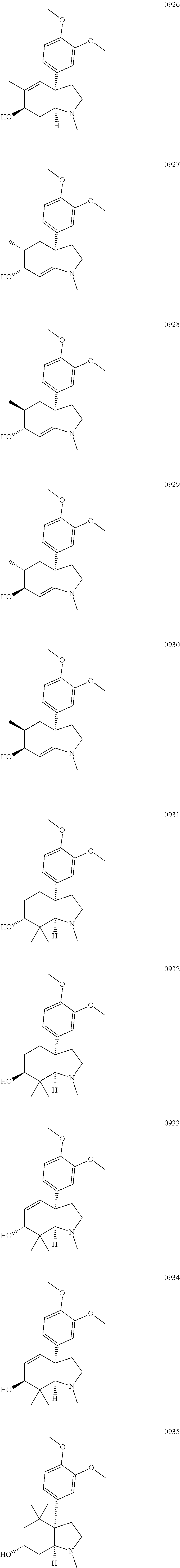

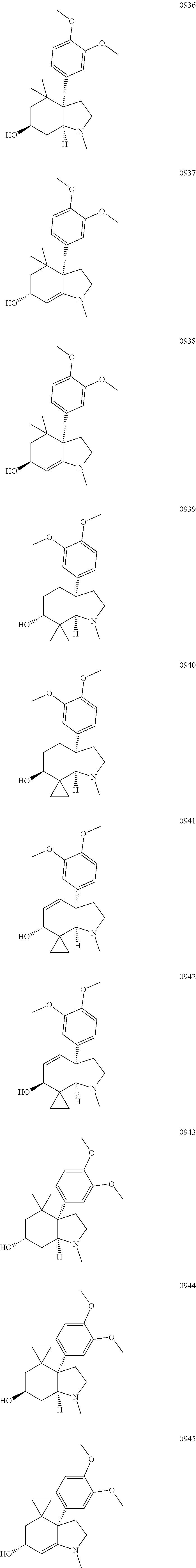

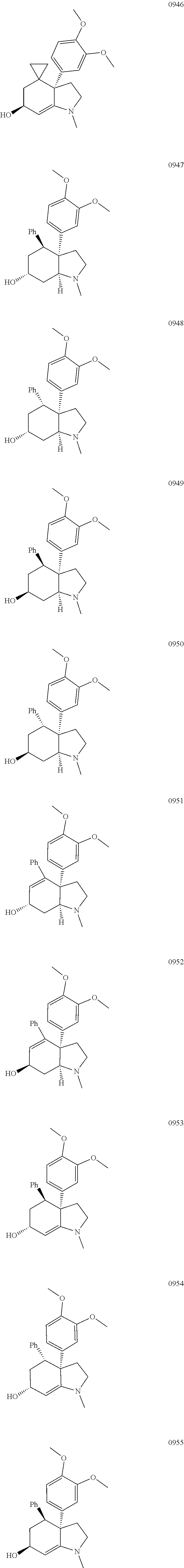

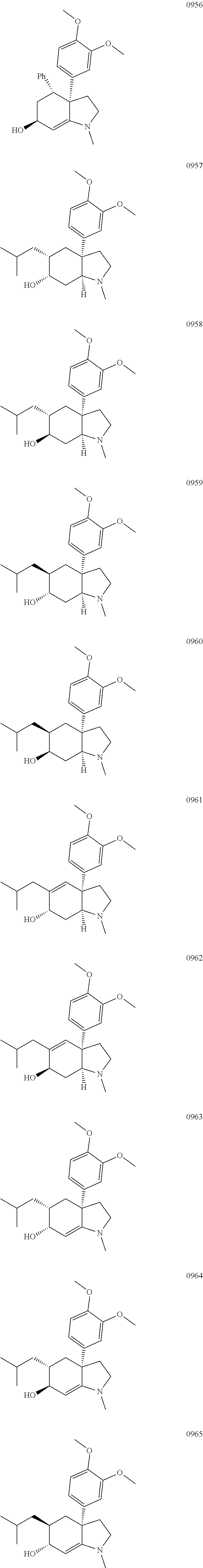

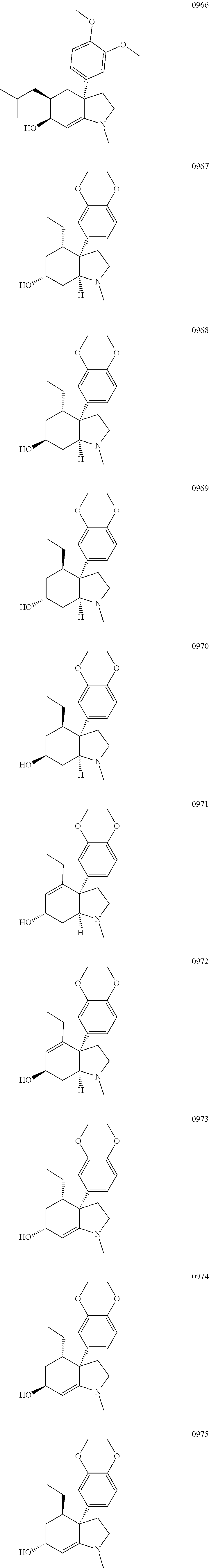

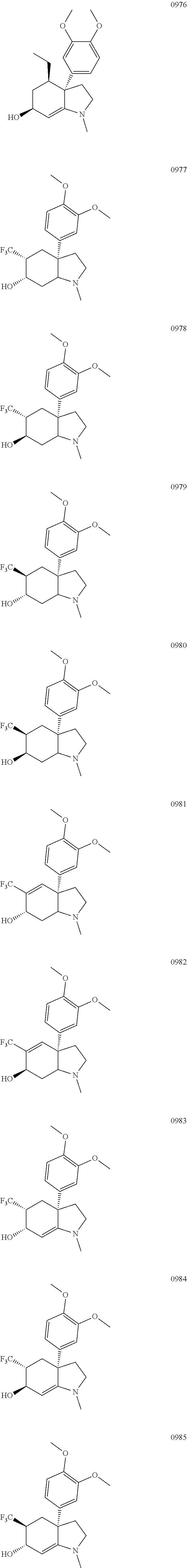

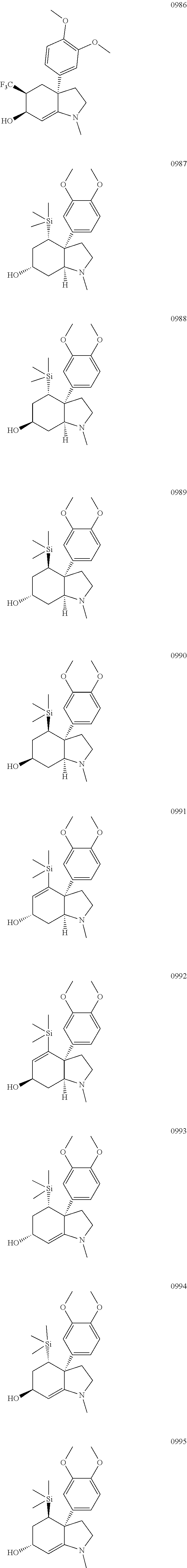

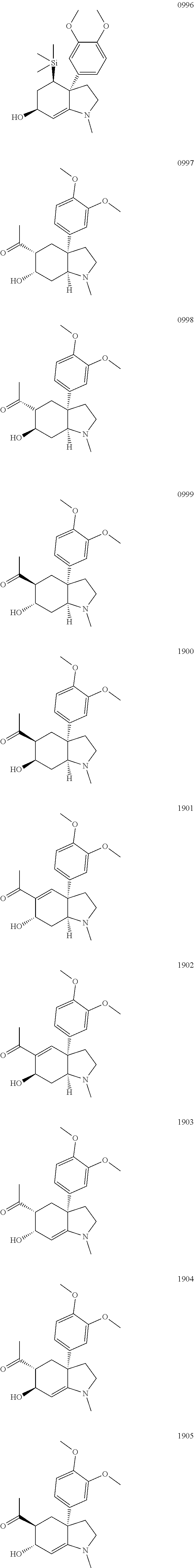

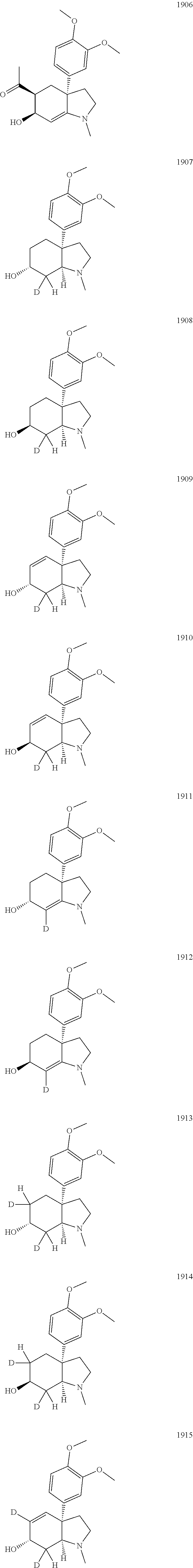

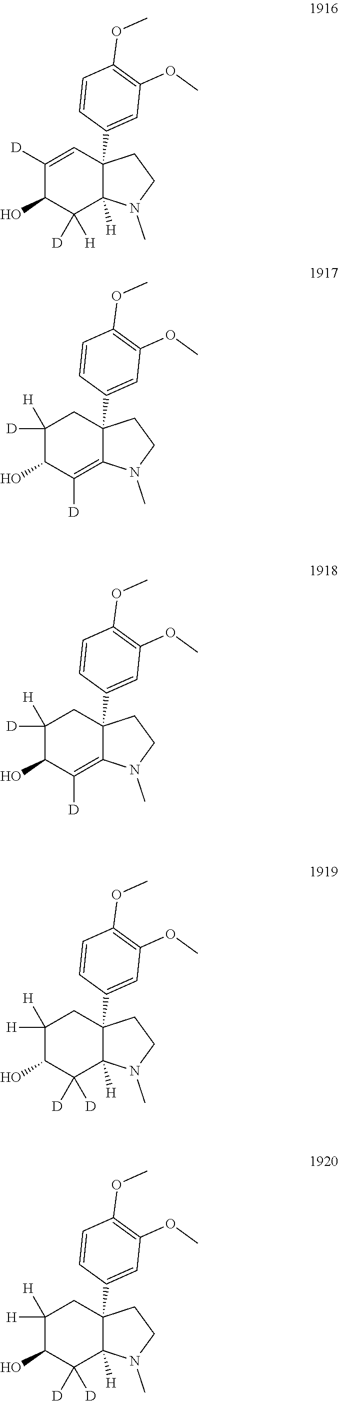

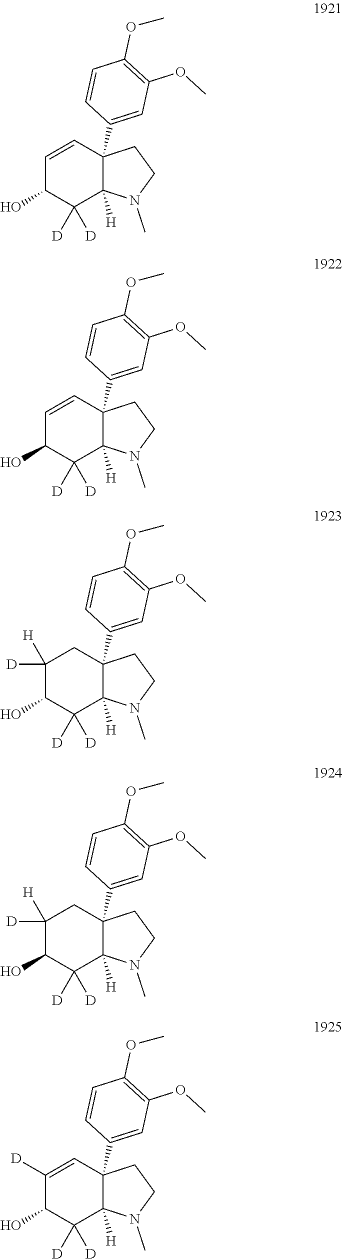

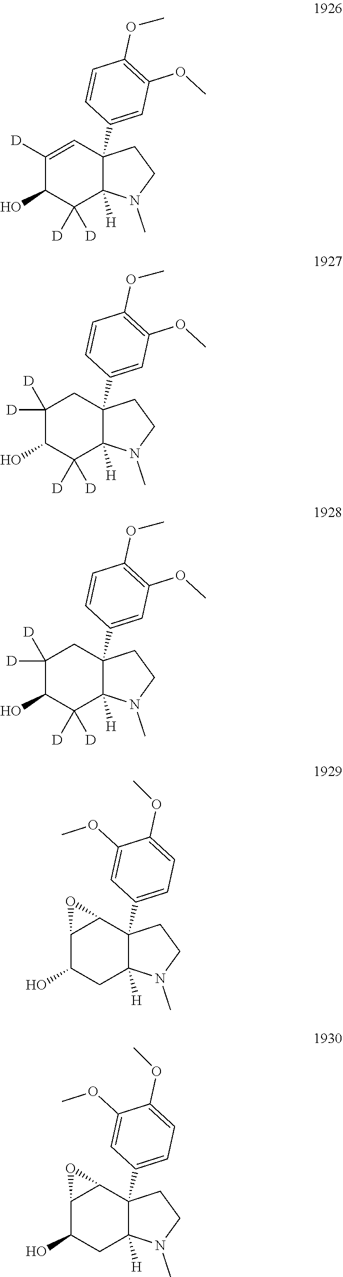

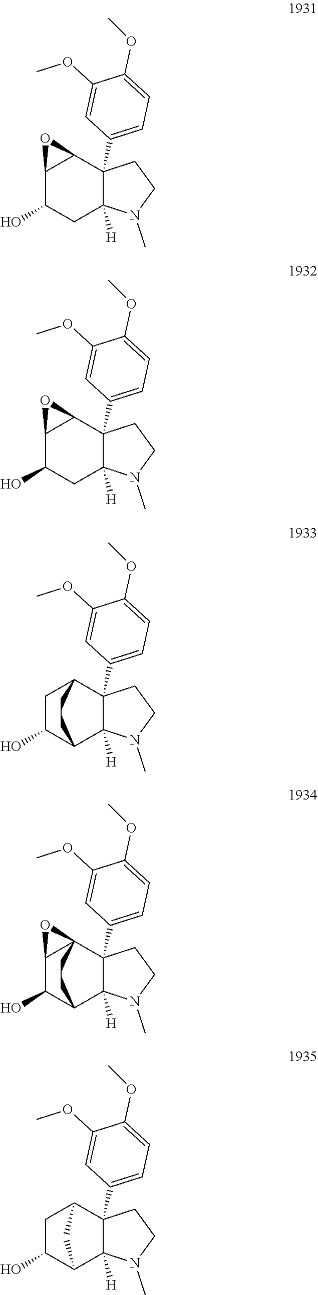

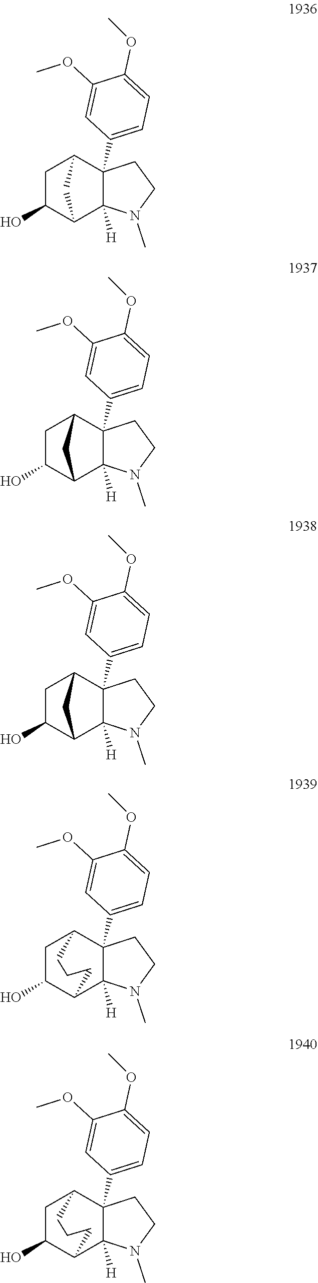

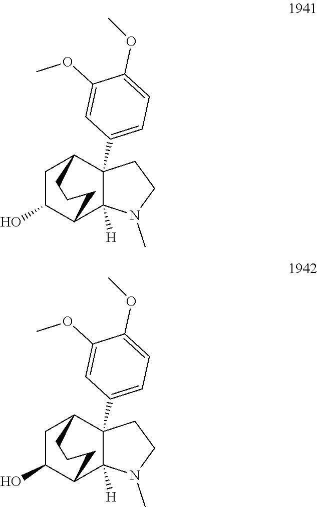

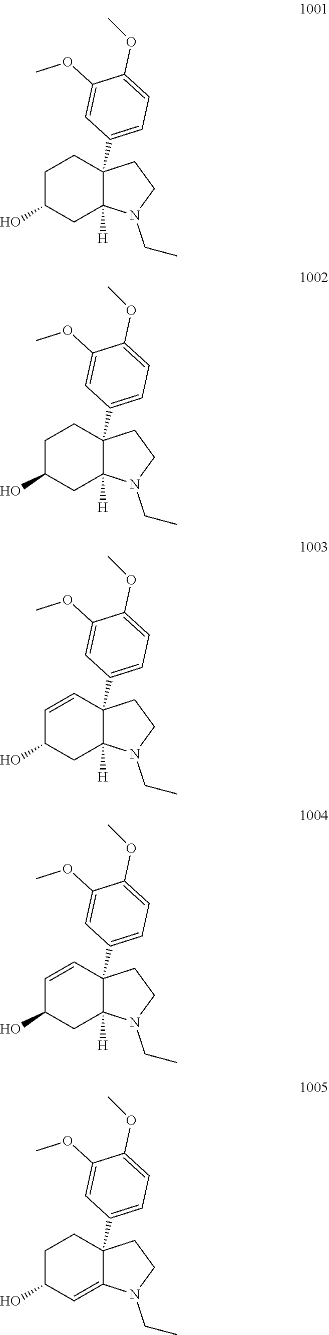 [SMILES: CCN1CC[C@]2(c3ccc(OC)c(OC)c3)CC[C@@H](O)C[C@H]12 ; CCN1CC[C@]2(c3ccc(OC)c(OC)c3)CC[C@H](O)C[C@H]12 ; CCN1CC[C@]2(c3ccc(OC)c(OC)c3)C=C[C@@H](O)C[C@H]12 ; CCN1CC[C@]2(c3ccc(OC)c(OC)c3)C=C[C@H](O)C[C@H]12 ; CCN1CC[C@]2(c3ccc(OC)c(OC)c3)CC[C@@H](O)C=C12]

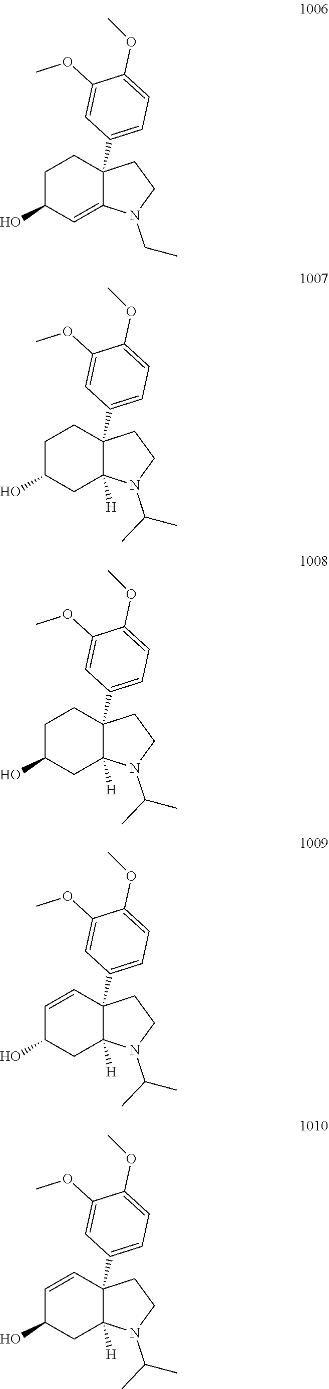

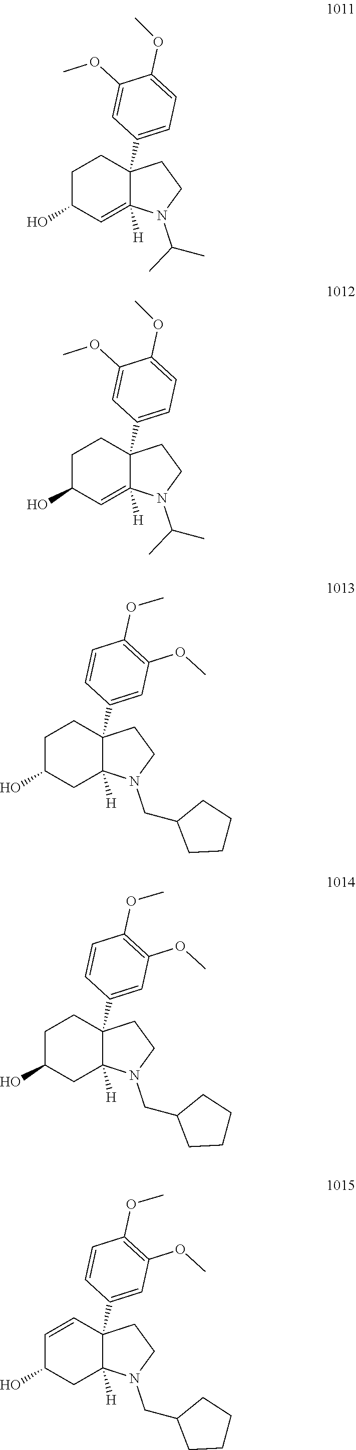

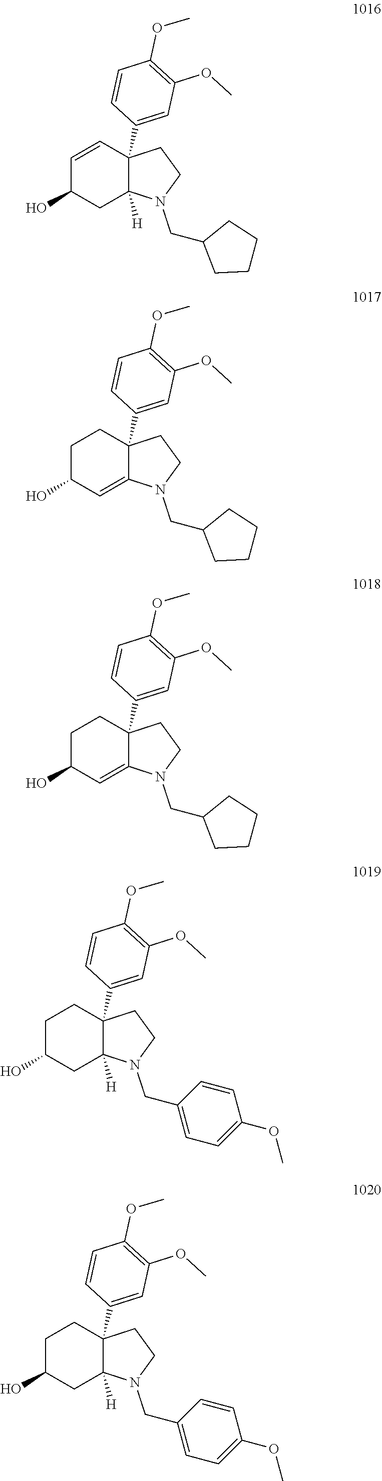

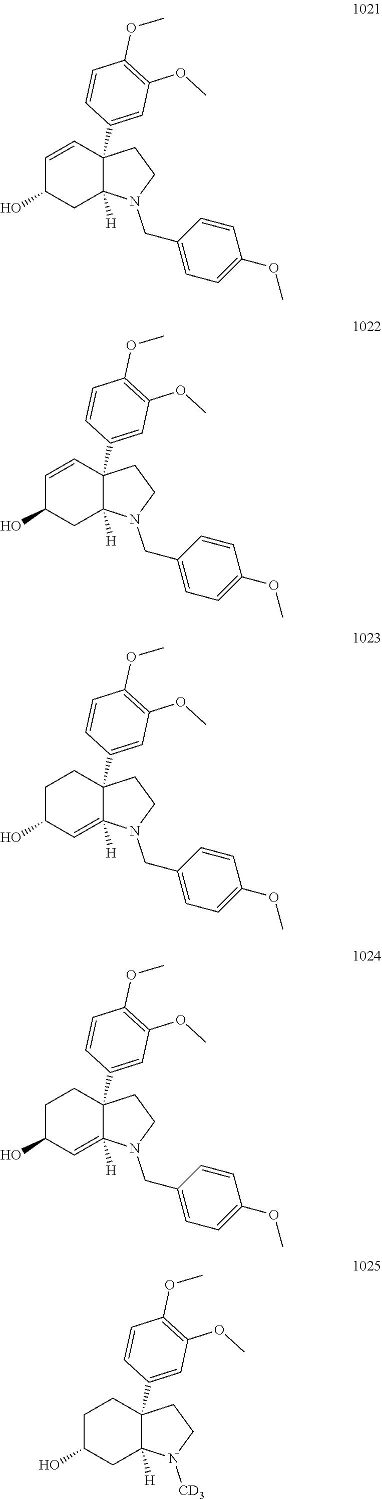

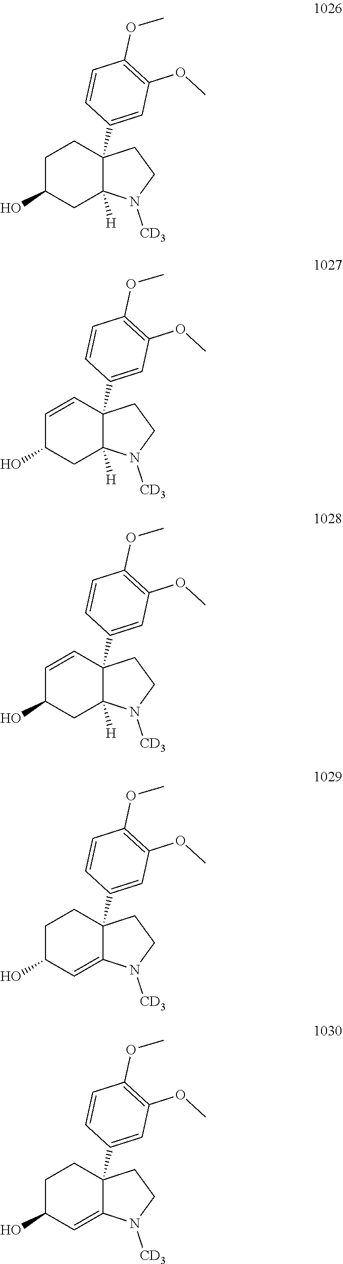

In some embodiments, the compound is

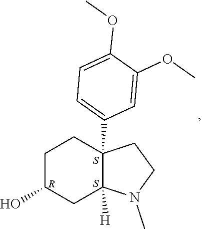

or a pharmaceutically acceptable salt thereof.

In some embodiments, the compound is

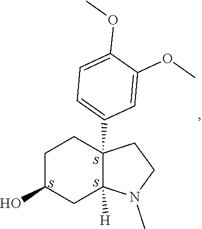

or a pharmaceutically acceptable salt thereof.

In some embodiments, the compound is

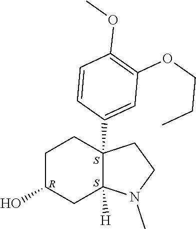

or a pharmaceutically acceptable salt thereof.

In some embodiments, the compound is

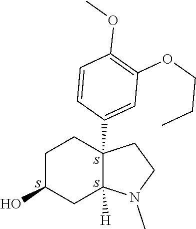

or a pharmaceutically acceptable salt thereof.

In some embodiments, the compound is

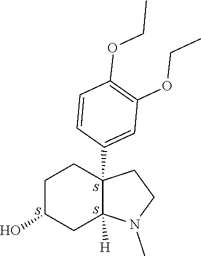

or a pharmaceutically acceptable salt thereof.

In some embodiments, the compound is the compound is

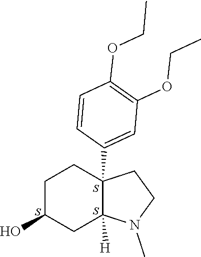

or a pharmaceutically acceptable salt thereof.

In some embodiments, the compound is

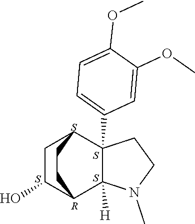

or a pharmaceutically acceptable salt thereof.

In some embodiments, the compound is

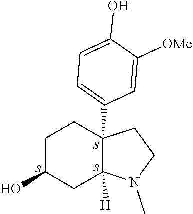

or a pharmaceutically acceptable salt thereof.

In some embodiments, the compound is

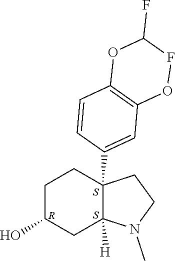

or a pharmaceutically acceptable salt thereof.

In some embodiments, the compound is

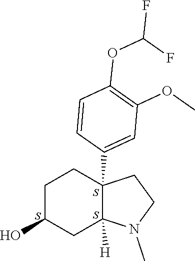

or a pharmaceutically acceptable salt thereof.

In some embodiments, the compound is the compound has the absolute stereochemistry shown.

In some embodiments, a pharmaceutical composition is provided, comprising a compound disclosed herein; and a pharmaceutical acceptable excipient.

In some embodiments, a method of treating a mental health disorder is provided, the method comprising administering to a mammal in need thereof an effective amount of the compounds described herein, or a pharmaceutically acceptable salt thereof. In some embodiments, the mental health disorder is anxiety, stress, or depression. In some embodiments, the mental health disorder is anxiety. In some embodiments, the mental health disorder is stress. In some embodiments, the mental health disorder is depression. In some embodiments, the mammal is a human.

In certain embodiments, the present disclosure provides a method of treating a central nervous condition, comprising administering to a subject in need thereof an effective amount of a compound of the present disclosure.

In certain embodiments, the present disclosure provides a method of treating a condition by administering a SERT inhibitor, comprising administering to a subject in need thereof an effective amount a compound of the present disclosure.

Numerous embodiments are further provided that can be applied to any aspect of the present invention described herein.

### DETAILED DESCRIPTION

The present invention is based, at least in part, on analogs of mesembranol, 6-epi-mesembranol and mesembrenol. Although mesembranol, 6-epi-mesembranol and mesembrenol are bioactive with certain desirable pharmacological effects, certain other properties are less than ideal for use as a therapeutic. For example, mesembranol and 6-epi-mesembranol demonstrate enhanced metabolic stability compared to mesembrine, but show a decrease in potency at 5-HTT. Leveraging the desirable metabolic stabilities of mesembranol and 6-epi-mesembranol, analogs more suitable for therapeutic development with increased potency at 5-HTT have been developed, and are disclosed herein.

### Compounds of the Invention

In certain aspects, the invention relates to compounds Formula (IA), (IB) or (IC):

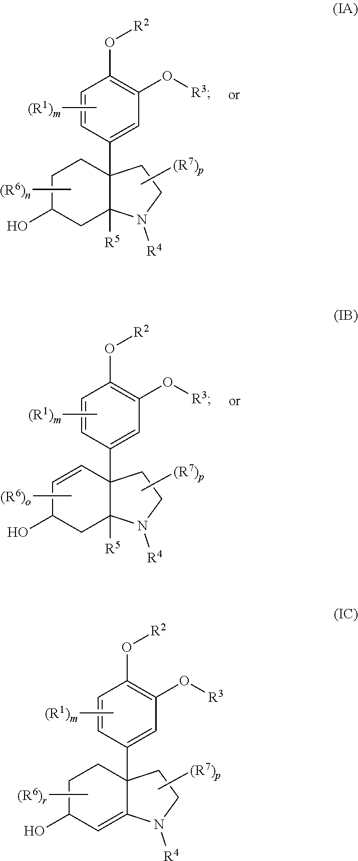

or a pharmaceutically acceptable salt thereof; wherein
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R⁴ is H or alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a};
R⁵ is H, deuterium, or methyl;
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form cycloalkyl, heterocyclyl or heteroaryl, wherein each hydrogen atom in cycloalkyl, heterocyclyl, and 5 heteroaryl is optionally substituted by halo, hydroxy, alkoxy, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, alkyl, —OH, or —NH₂; wherein each hydrogen atom in alkyl is optionally substituted by —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl;
provided the compound is not:

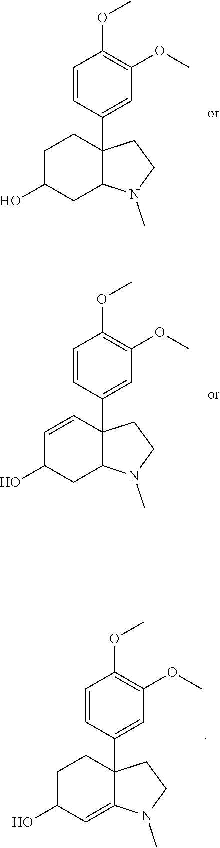 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

In certain embodiments, the compound is of Formula (IA-a), (IA-b), (IB-a), (IB-b), (IC-a) or (IC-b):

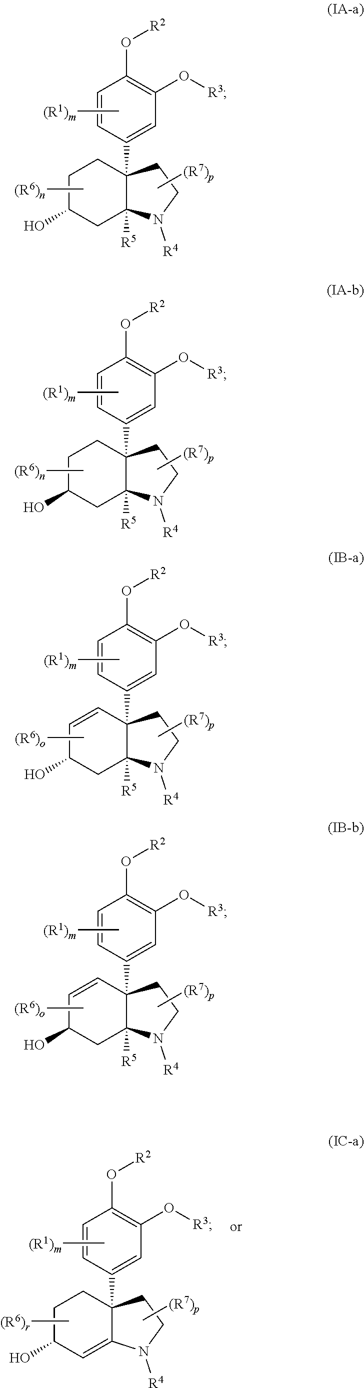

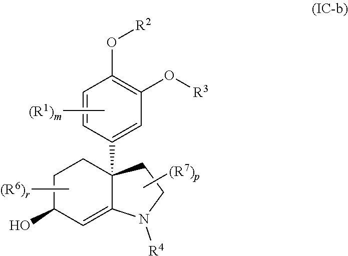

or a pharmaceutically acceptable salt thereof; wherein R¹, R², R³, R⁴, R⁵, R⁶, R⁷, n, m, n, o, p and r are as disclosed for Formula (IA), Formula (IB) and Formula (IC), respectively,
provided the compound is not:

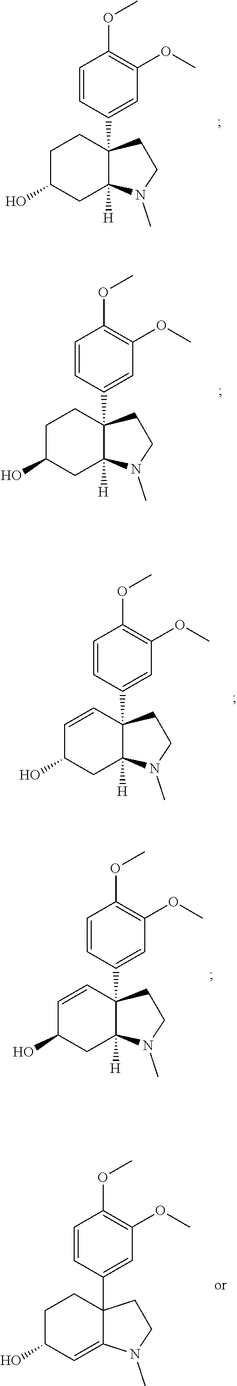 [SMILES: COc1ccc([C@@]23CC[C@@H](O)C[C@@H]2N(C)CC3)cc1OC ; COc1ccc([C@@]23CC[C@H](O)C[C@@H]2N(C)CC3)cc1OC ; COc1ccc([C@@]23C=C[C@@H](O)C[C@@H]2N(C)CC3)cc1OC ; COc1ccc([C@@]23C=C[C@H](O)C[C@@H]2N(C)CC3)cc1OC ; COc1ccc(C23CC[C@@H](O)C=C2N(C)CC3)cc1OC]

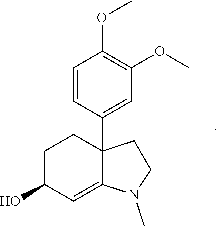 [SMILES: COc1ccc(C23CC[C@H](O)C=C2N(C)CC3)cc1OC]

In certain embodiments, the compound is of Formula (IA-c), (IB-c), or (IC-c):

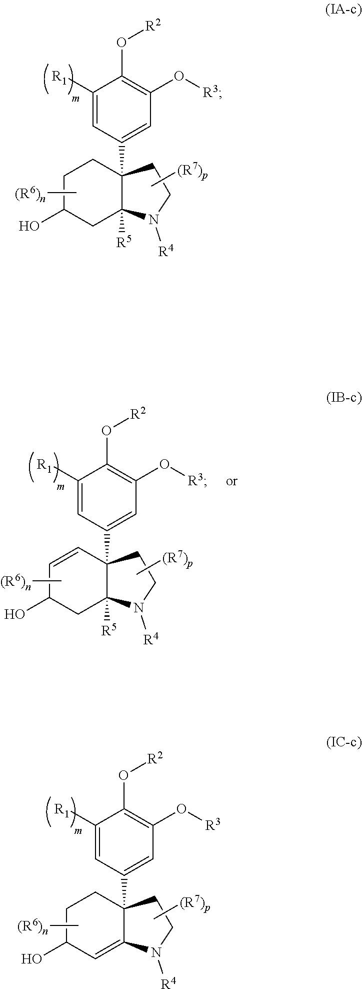

or a pharmaceutically acceptable salt thereof; wherein R¹, R₂, R₃, R₄, R₅, R₆, R₇, n, o, p and r are as disclosed for Formula (IA), Formula (IB) and Formula (IC), respectively, m is 0 or 1, and provided the compound is not:

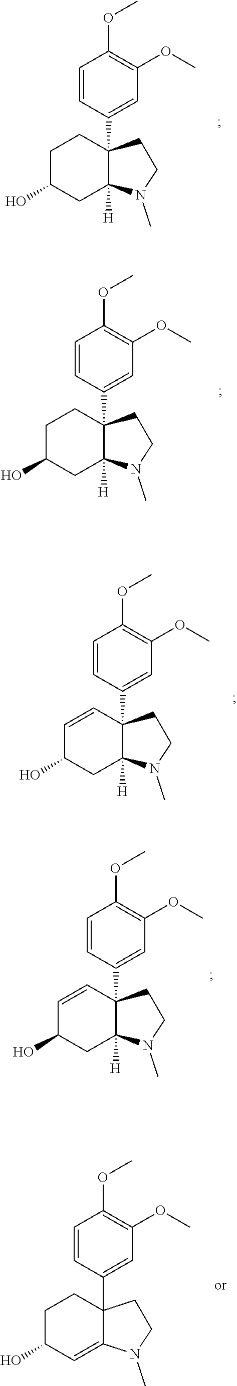 [SMILES: COc1ccc([C@@]23CC[C@@H](O)C[C@@H]2N(C)CC3)cc1OC ; COc1ccc([C@@]23CC[C@H](O)C[C@@H]2N(C)CC3)cc1OC ; COc1ccc([C@@]23C=C[C@@H](O)C[C@@H]2N(C)CC3)cc1OC ; COc1ccc([C@@]23C=C[C@H](O)C[C@@H]2N(C)CC3)cc1OC ; COc1ccc(C23CC[C@@H](O)C=C2N(C)CC3)cc1OC]

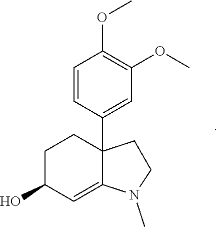 [SMILES: COc1ccc(C23CC[C@H](O)C=C2N(C)CC3)cc1OC]

In certain embodiments, the compound is of Formula (IA-c), (IB-c), or (IC-c):

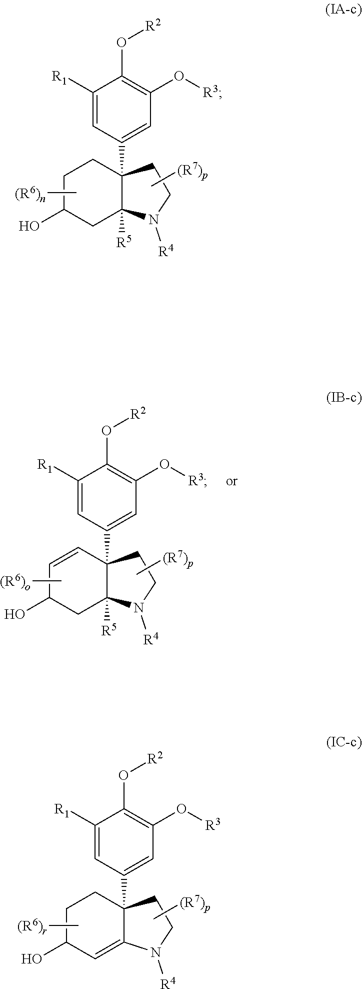

or a pharmaceutically acceptable salt thereof; wherein R⁴ is methyl, R⁵ is H, p is 0, and R₁, R₂, R₃, R₆, R₇, n, m, n, o, and r are as disclosed for Formula (IA), Formula (IB) and Formula (IC), respectively, and provided the compound is not:

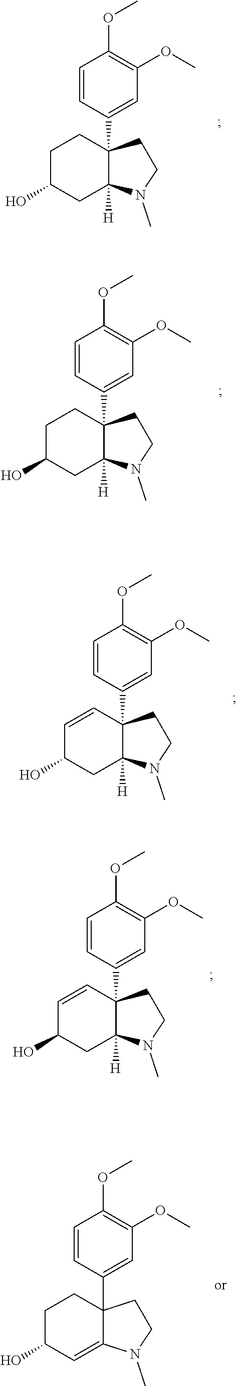 [SMILES: COc1ccc([C@@]23CC[C@@H](O)C[C@@H]2N(C)CC3)cc1OC ; COc1ccc([C@@]23CC[C@H](O)C[C@@H]2N(C)CC3)cc1OC ; COc1ccc([C@@]23C=C[C@@H](O)C[C@@H]2N(C)CC3)cc1OC ; COc1ccc([C@@]23C=C[C@H](O)C[C@@H]2N(C)CC3)cc1OC ; COc1ccc(C23CC[C@@H](O)C=C2N(C)CC3)cc1OC]

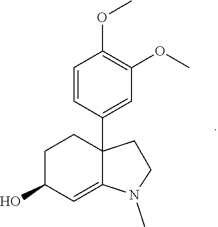 [SMILES: COc1ccc(C23CC[C@H](O)C=C2N(C)CC3)cc1OC]

In certain embodiments, the compounds of Formula (IA-a), (IA-b), (IA-c), (IB-a), (IB-b), (IB-c), (IC-a), (IC-b) and (IC-c) have the absolute stereochemistry shown.

In certain embodiments, the compound is of Formula (IA), Formula (IA-a), Formula (IA-b), or Formula (IA-c) or a pharmaceutically acceptable salt thereof.

In certain embodiments, the compound is of Formula (IB), Formula (IB-a), Formula (IB-b), or Formula (IB-c) or a pharmaceutically acceptable salt thereof.

In certain embodiments, the compound is of Formula (IC), Formula (IC-a), Formula (IC-b), or Formula (IC-c) or a pharmaceutically acceptable salt thereof.

In certain embodiments, R¹ is independently deuterium, halo, C₁₋₄ alkyl, C₂₋₄ alkenyl, C₂₋₄ alkynyl, C₃₋₈ cycloalkyl, C₃₋₈ cycloalkenyl, 3-8 membered heterocyclyl comprising one or more N, O or S heteroatoms, C₅₋₆ aryl, 5-6 membered heteroaryl comprising one or more N, O or S heteroatoms, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, and alkynyl, is optionally substituted by halo, C₁₋₄ alkyl, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; and wherein each hydrogen atom in cycloalkyl, cycloalkenyl, heterocyclyl, aryl, and heteroaryl is optionally substituted by halo, C₁₋₄ alkyl, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, R¹ is methyl. In certain embodiments, R¹ is ethyl. In certain embodiments, R¹ is methyl optionally substituted with one or more F. In certain embodiments, R¹ is CHF₂. In certain embodiments, R¹ is CF₃. In certain embodiments, R¹ is F. In certain embodiments, R¹ is Cl. In certain embodiments, R¹ is Br. In certain embodiments, R¹ is I. In certain embodiments, R¹ is —C(O)—NR^{a}R^{b}. In certain embodiments, R¹ is —C(O)—NH₂. In certain embodiments, R¹ is cyclopropyl. In certain embodiments, R¹ is —(CH₂)-cycloprpopyl. In certain embodiments, R¹ is methoxy. In certain embodiments, R¹ is —CH═CH₂. In certain embodiments, R¹ is —CH₂—CH═CH₂.

In certain embodiments, each of R² and R³ is independently H, C₁₋₄ alkyl, C₃₋₈ cycloalkyl, C₂₋₄ alkenyl, C₂₋₄ alkynyl, or C₅₋₆ aryl; wherein each hydrogen atom in alkyl, alkenyl, and alkynyl, is optionally substituted by halo, deuterium, C₃₋₈ cycloalkyl, C₅₋₆ aryl, or OR^{a}; wherein each hydrogen atom in cycloalkyl, and aryl is optionally substituted by halo, deuterium, C₁₋₄ alkyl, C₁₋₄ haloalkyl, or OR^{a}.

In certain embodiments, R² and R³ are each independently C₁₋₄ alkyl. In certain embodiments, each of R² and R³ is independently methyl. In certain embodiments, R² is methyl or ethyl and R³ is methyl, or ethyl. In certain embodiments, R² is ethyl, and R³ is ethyl. In certain embodiments, R² is methyl, and R³ is ethyl. In certain embodiments, R² is ethyl, and R³ is methyl.

In certain embodiments, each of R² and R³ is independently C₁₋₄ alkyl substituted with one or more deuterium. In certain embodiments, each of R² and R³ is independently —CD₃. In certain embodiments, R² is methyl and R³ is —CD₃. In certain embodiments, R³ is methyl and R² is —CD₃.

In certain embodiments, R² and R³ are each independently C₁₋₄ alkyl or C₁₋₄ alkyl substituted with one or more halogen. In certain embodiments, R² and R³ are each independently C₁₋₄ alkyl or C₁₋₄ alkyl substituted with one or more F. In certain embodiments, R² is methyl and R³ is C₁₋₄ alkyl substituted with one or more halogen. In certain embodiments, R³ is methyl and R² is C₁₋₄ alkyl substituted with one or more halogen.

In certain embodiments, R² is methyl and R³ is —CF₃. In certain embodiments, R³ is methyl and R² is —CF₃. In certain embodiments, R² and R³ are each —CF₃. In certain embodiments, R² is methyl and R³ is —CHF₂. In certain embodiments, R³ is methyl and R² is —CHF₂. In certain embodiments, R² and R³ are each —CHF₂.

In certain embodiments, R² is C₁₋₄ alkoxy and R³ is C₁₋₄ alkyl or C₁₋₄ alkoxy. In certain embodiments, R² is methyl and R³ is —(CH₂—O)ᵥ—CH₃ wherein v is 1, 2 or 3. In certain embodiments, R³ is methyl and R² is —(CH₂—O)ᵥ—CH₃ wherein v is 1, 2 or 3. In certain embodiments, R² and R³ are each independently —(CH₂—O)ᵥ—CH₃ wherein each v is independently 1, 2 or 3. In certain embodiments, R² and R³ are each independently C₁₋₄ alkyl.

In certain embodiments, R² and R³ are each independently C₁₋₄ alkyl, cyclopropyl, cyclobutyl or cyclohexyl. In certain embodiments, R² is methyl and R³ is cyclopropyl. In certain embodiments, R³ is methyl and R² is cyclopropyl. In certain embodiments, R² is cyclopropyl and R³ is cyclopropyl. In certain embodiments, R² is methyl and R³ is —(CH₂)-cyclopropyl. In certain embodiments, R² and R³ are each —(CH₂)-cyclopropyl. In certain embodiments, R² is methyl and R³ is cyclobutyl. In certain embodiments, R³ is methyl and R² is cyclobutyl. In certain embodiments, R² is cyclobutyl and R³ is cyclobutyl. In certain embodiments, R² is methyl and R³ is —(CH₂)-cyclobutyl. In certain embodiments, R² and R³ are each —(CH₂)-cyclobutyl. In certain embodiments, R² is methyl and R³ is cyclohexyl. In certain embodiments, R³ is methyl and R² is cyclohexyl. In certain embodiments, R² is cyclohexyl and R³ is cyclohexyl. In certain embodiments, R² is methyl and R³ is —(CH₂)-cyclohexyl. In certain embodiments, R² and R³ are each —(CH₂)-cyclohexyl. In certain embodiments, R² is methyl and R³ is cyclobutyl. In certain embodiments, R² is methyl and R³ is cyclohexyl. In certain embodiments, R² is methyl and R³ is benzyl.

In certain embodiments, R² and R³ together with the atoms to which they are attached combine to form 3-8 membered heterocyclyl comprising one or more N, O or S heteroatoms, or 5-6 membered heteroaryl comprising one or more N, O or S heteroatoms, wherein each hydrogen atom in heterocyclyl and heteroaryl is optionally substituted by halo, C₁₋₄ alkyl, C₁₋₄ haloalkyl, or OR^{a}. In certain embodiments, R² and R³ together with the atoms to which they are attached combine to form 5-membered heterocyclyl comprising one or more N, O or S heteroatoms, optionally substituted with one or more halo, C₁₋₄ alkyl, C₁₋₄ haloalkyl, or C₁₋₄ alkoxy. In certain embodiments, R² and R³ together with the atoms to which they are attached combine to form 5-membered heterocyclyl comprising one or more oxygen heteroatoms, optionally substituted with one or more F, methyl or methoxy. In certain embodiments, R² and R³ together with the atoms to which they are attached combine to form 6-membered heterocyclyl comprising one or more N, O or S heteroatoms, optionally substituted with one or more halo, C₁₋₄ alkyl, C₁₋₄ haloalkyl, or C₁₋₄ alkoxy. In certain embodiments, R² and R³ together with the atoms to which they are attached combine to form 6-membered heterocyclyl comprising one or more oxygen heteroatoms, optionally substituted with one or more F, methyl or methoxy. In certain embodiments, R² and R³ together with the atoms to which they are attached combine to form 6-membered heterocyclyl comprising one or more N, O or S heteroatoms, optionally substituted with one or more halo, C₁₋₄ alkyl, C₁₋₄ haloalkyl, or C₁₋₄ alkoxy. In certain embodiments, R² and R³ together with the atoms to which they are attached combine to form 6-membered heterocyclyl comprising one or more oxygen heteroatoms, optionally substituted with one or more F, methyl or methoxy.

In certain embodiments, R⁴ is H or C₁₋₄ alkyl, wherein each hydrogen atom in alkyl is optionally substituted by halo, deuterium, C₃₋₈ cycloalkyl, C₅₋₆ aryl, —OR^{a}, or —NR^{a}R^{b}; wherein each hydrogen atom in aryl is optionally substituted by OR^{a}. In certain embodiments, R⁴ is methyl.

In certain embodiments, R⁵ is H, deuterium, or methyl. In certain embodiments, R⁵ is H. In certain embodiments, R⁵ is deuterium.

In certain embodiments, each R⁶ is independently deuterium, C₁₋₄ alkyl, C₃₋₈ cycloalkyl, —Si(C₁₋₄ alkyl)₃, —C(O)—(C₁₋₄)-alkyl, C₅₋₆ aryl, 5-6 membered heteroaryl comprising one or more N, O or S heteroatoms, or 3-8 membered heterocyclyl comprising one or more N, O or S heteroatoms; wherein each hydrogen atom in alkyl, —Si(alkyl)₃, and —C(O)alkyl is optionally substituted by halo, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; and wherein each hydrogen atom in cycloalkyl, phenyl, heteroaryl, and heterocyclyl is optionally substituted by halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, each R⁶ is methyl.

In certain embodiments, two R⁶ substituents on alkyl, cycloalkyl, —Si(alkyl)₃, —C(O)alkyl, phenyl, heteroaryl, and heterocyclyl together with the atoms to which they are attached combine to form C₅₋₆ aryl, C₃₋₈ cycloalkyl, 5-6 membered heteroaryl comprising one or more N, O or S heteroatoms, or 3-8 membered heterocyclyl comprising one or more N, O or S heteroatoms.

In certain embodiments, two R⁶s on different carbon atoms together with the carbon atoms to which they are attached combine to form a C₃₋₈ cycloalkyl, a 3-8 membered heterocyclyl comprising one or more N, O or S heteroatoms, or a 5-6 membered heteroaryl comprising one or more N, O or S heteroatoms, wherein each hydrogen atom in cycloalkyl, heterocyclyl, and heteroaryl is optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on different carbon atoms together with the carbon atoms to which they are attached combine to form a bridging C₃₋₈ cycloalkyl, or a bridging 3-8 membered heterocyclyl comprising one or more N, O or S heteroatoms, wherein each hydrogen atom in cycloalkyl, or heterocyclyl is optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}.

In certain embodiments, two R⁶s on different carbon atoms together with the carbon atoms to which they are attached combine to form a bridging C₅ cycloalkyl, or a bridging 5 membered heterocyclyl comprising one or more N, O or S heteroatoms, wherein each hydrogen atom in cycloalkyl, or heterocyclyl, is optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on different carbon atoms together with the carbon atoms to which they are attached combine to form a bridging C₆ cycloalkyl, or a bridging 6 membered heterocyclyl comprising one or more N, O or S heteroatoms, wherein each hydrogen atom in cycloalkyl, or heterocyclyl, is optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on different carbon atoms together with the carbon atoms to which they are attached combine to form a bridging C₇ cycloalkyl, or a bridging 7 membered heterocyclyl comprising one or more N, O or S heteroatoms, wherein each hydrogen atom in cycloalkyl, or heterocyclyl, is optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on different carbon atoms together with the carbon atoms to which they are attached combine to form a bridging C₈ cycloalkyl, or a bridging 8 membered heterocyclyl comprising one or more N, O or S heteroatoms, wherein each hydrogen atom in cycloalkyl, or heterocyclyl, is optionally substituted by halo, hydroxy, C₁₋₄ alkyl optionally substituted by one or more halo, C₁₋₄ alkanol, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on different carbon atoms together with the carbon atoms to which they are attached combine to form a bridging C₅₋₈ unsubstituted cycloalkyl. In certain embodiments, two R⁶s on different carbon atoms together with the carbon atoms to which they are attached combine to form a bridging C₅ unsubstituted cycloalkyl. In certain embodiments, two R⁶s on different carbon atoms together with the carbon atoms to which they are attached combine to form a bridging C₆ unsubstituted cycloalkyl. In certain embodiments, two R⁶s on different carbon atoms together with the carbon atoms to which they are attached combine to form a bridging C₇ unsubstituted cycloalkyl. In certain embodiments, two R⁶s on different carbon atoms together with the carbon atoms to which they are attached combine to form a bridging C₈ unsubstituted cycloalkyl.

In certain embodiments, two R⁶s on different carbon atoms together with the carbon atoms to which they are attached combine to form a fused C₅₋₆ cycloalkyl or a fused 5-6 membered heteroaryl comprising one or more N, O or S heteroatoms, wherein each hydrogen atom in the heteroaryl is optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R's on different carbon atoms together with the carbon atoms to which they are attached combine to form a fused C₅₋₆ cycloalkyl, optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on different carbon atoms together with the carbon atoms to which they are attached combine to form a fused 5-6 membered heteroaryl comprising one or more N, O or S heteroatoms, wherein each hydrogen atom in the heteroaryl is optionally substituted by halo, hydroxy, C₁₋₄ alkyl optionally substituted by one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}.

In certain embodiments, two R⁶s on a single carbon atom together with the carbon atom to which they are attached combine to form a C₃₋₈ cycloalkyl, 3-8 membered heterocyclyl comprising one or more N, O or S heteroatoms, or 5-6 membered heteroaryl comprising one or more N, O or S heteroatoms, wherein each hydrogen atom in cycloalkyl, heterocyclyl, and 5-6 membered heteroaryl comprising one or more N, O or S heteroatoms is optionally substituted by halo, hydroxy, C₁₋₄ alkoxy, C₁₋₄ alkanol, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on a single carbon atom together with the carbon atom to which they are attached combine to form a spirocyclic C₃₋₈ cycloalkyl, optionally substituted by halo, hydroxy, C₁₋₄ alkoxy, C₁₋₄ alkanol, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on a single carbon atom together with the carbon atom to which they are attached combine to form a spirocyclic C₃ cycloalkyl, optionally substituted by halo, hydroxy, C₁₋₄ alkoxy, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on a single carbon atom together with the carbon atom to which they are attached combine to form a spirocyclic C₄ cycloalkyl, optionally substituted by halo, hydroxy, C₁₋₄ alkoxy, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on a single carbon atom together with the carbon atom to which they are attached combine to form a spirocyclic C₅ cycloalkyl, optionally substituted by halo, hydroxy, C₁₋₄ alkoxy, C₁₋₄ alkanol, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}. In certain embodiments, two R⁶s on a single carbon atom together with the carbon atom to which they are attached combine to form a spirocyclic C₆ cycloalkyl, optionally substituted by halo, hydroxy, C₁₋₄ alkoxy, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}.

In certain embodiments, each R⁷ is independently deuterium, C₁₋₄ alkyl, C₁₋₄ haloalkyl, —OH, or —NH₂; wherein each hydrogen atom in alkyl is optionally substituted by —OR^{a} or —NR^{a}R^{b}.

In certain embodiments, m is 0, 1, 2, or 3.

In certain embodiments, n is 0, 1, 2, 3, 4, 5, or 6. In certain embodiments, n is 0.

In certain embodiments, o is 0, 1, 2, 3, or 4.

In certain embodiments, p is 0, 1, 2, 3, or 4. In certain embodiments, p is 0.

In certain embodiments, r is 0, 1, 2, 3, 4, or 5.

In certain embodiments, p is 0 and n is 0.

In certain embodiments, each R^{a} and R^{b} is independently H, C₁₋₄ alkyl, C₂₋₄ alkenyl, C₃₋₈ cycloalkyl, 3-8 membered heterocyclyl comprising one or more N, O or S heteroatoms, C₅₋₆ aryl, or 5-6 membered heteroaryl. In certain embodiments, each R^{a} and R^{b} is independently H or methyl. In certain embodiments, each R^{a} and R^{b} is independently H, C₁₋₄ alkyl, or C₂₋₄ alkenyl. In certain embodiments, each R^{a} is H and R^{b} is methyl. In certain embodiments, each R^{a} is methyl and R^{b} is H. In certain embodiments, each R^{a} and R^{b} is methyl. In certain embodiments, each R^{a} and R^{b} is independently H.

In certain embodiments, if an instance of R¹ is —NR^{a}R^{b}, then R^{a} and R^{b} may combine with the nitrogen atom to which they are attached to form 3-8 membered heterocyclyl or 5-6 membered heteroaryl; wherein each hydrogen atom in alkyl, or alkenyl, is optionally substituted by halo, hydroxy, C₁₋₄ alkyl, C₁₋₄ alkanol, C₅₋₆ aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; wherein each hydrogen atom in cycloalkyl, heterocyclyl, aryl, or heteroaryl is optionally substituted by halo, hydroxy, C₁₋₄ alkyl, C₁₋₄ haloalkyl, C₁₋₄ alkanol, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and/or
each R^{c} and R^{d} is independently H, C₁₋₄ alkyl, C₂₋₄ alkenyl, C₃₋₈ cycloalkyl, 3-8 membered heterocyclyl comprising one or more N, O or S heteroatoms, C₅₋₆ aryl, or 5-6 membered heteroaryl comprising one or more N, O or S heteroatoms;
provided the compound is not:

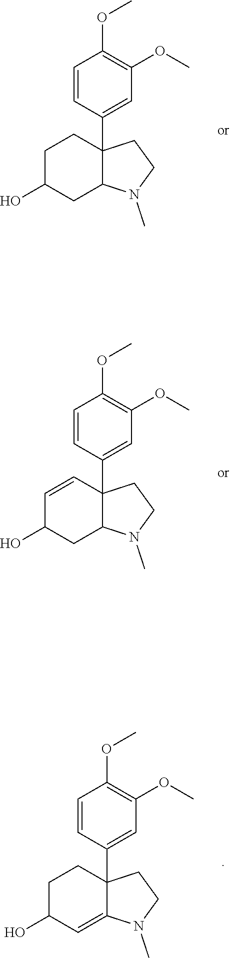 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

In certain embodiments, R⁵ is H or methyl. In certain embodiments, R⁵ is methyl.

In certain embodiments, the compound is selected from the group consisting of:

TABLE 1
Compounds of Formula IA-a, IA-b, IB-a and IB-b

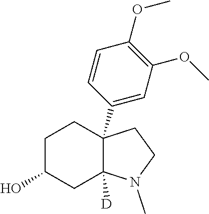 [SMILES: [2H][C@]12C[C@H](O)CC[C@@]1(c1ccc(OC)c(OC)c1)CCN2C]

0096

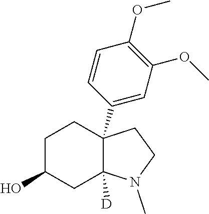 [SMILES: [2H][C@]12C[C@@H](O)CC[C@@]1(c1ccc(OC)c(OC)c1)CCN2C]

0097

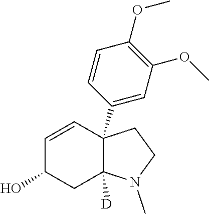 [SMILES: [2H][C@]12C[C@H](O)C=C[C@@]1(c1ccc(OC)c(OC)c1)CCN2C]

0098

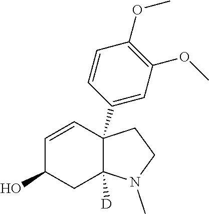 [SMILES: [2H][C@]12C[C@@H](O)C=C[C@@]1(c1ccc(OC)c(OC)c1)CCN2C]

0099

or a pharmaceutically acceptable salt thereof.

In certain embodiments, the compound is of Formula (IA-1) or (IB-1):

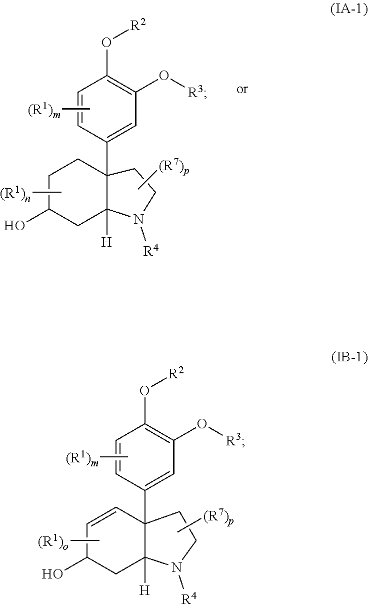

or a pharmaceutically acceptable salt thereof.

In certain embodiments, the compound is of Formula (IA-1) or a pharmaceutically acceptable salt thereof.

In certain embodiments, the compound is of Formula (IB-1) or a pharmaceutically acceptable salt thereof.

In certain embodiments, the compound is of Formula (IA-1a), (IA-1b), (IB-1a), or (IB-1b):

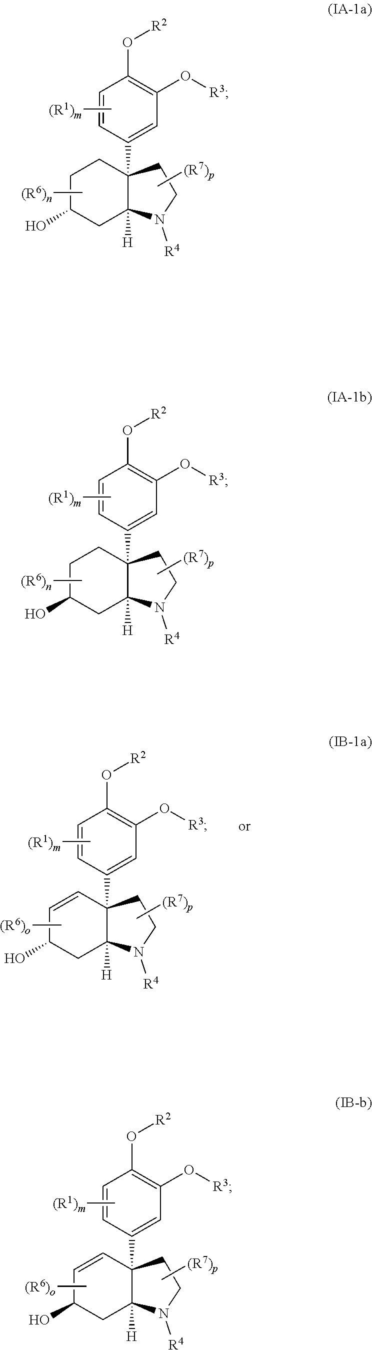

or a pharmaceutically acceptable salt thereof; wherein

In certain embodiments, the compounds of Formula (IA-1a), (IA-1b), (IB-1a), and (IB-1b) have the absolute stereochemistry shown.

In certain embodiments, the compound is of Formula (IA-1a) or a pharmaceutically acceptable salt thereof.

In certain embodiments, the compound is of Formula (IA-1b) or a pharmaceutically acceptable salt thereof.

In certain embodiments, the compound is of Formula (IB-1a) or a pharmaceutically acceptable salt thereof.

In certain embodiments, the compound is of Formula (IB-1b) or a pharmaceutically acceptable salt thereof.

In certain embodiments, p is 1.

In certain embodiments, R⁷ is C₁-C₆ alkyl such as methyl or ethyl.

In certain embodiments, the compound is selected from the group consisting of:

TABLE 2
Compounds of Formula IA-a, IA-b, IB-a and IB-b

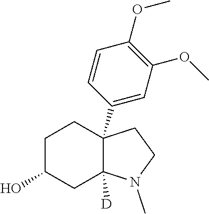 [SMILES: [2H][C@]12C[C@H](O)CC[C@@]1(c1ccc(OC)c(OC)c1)CCN2C]

0096

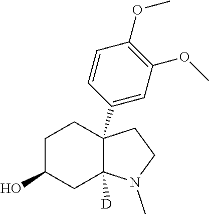 [SMILES: [2H][C@]12C[C@@H](O)CC[C@@]1(c1ccc(OC)c(OC)c1)CCN2C]

0097

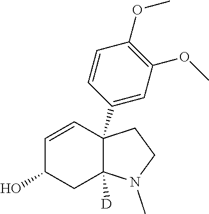 [SMILES: [2H][C@]12C[C@H](O)C=C[C@@]1(c1ccc(OC)c(OC)c1)CCN2C]

0098

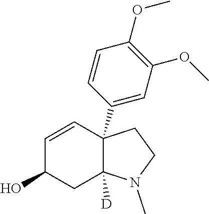 [SMILES: [2H][C@]12C[C@@H](O)C=C[C@@]1(c1ccc(OC)c(OC)c1)CCN2C]

0099

or a pharmaceutically acceptable salt thereof.

In certain embodiments, the compound is of Formula (IA-2), (IB-2) or (IC-2):

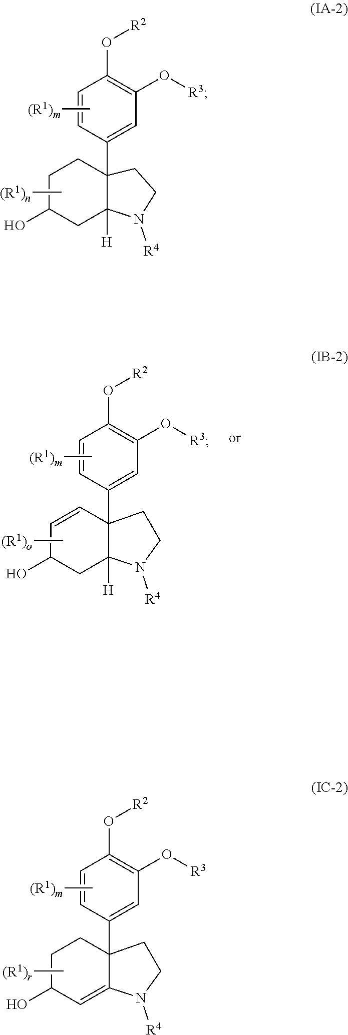

or a pharmaceutically acceptable salt thereof.

In certain embodiments, the compound is of Formula (IA-2c), (IB-2c) or (IC-2c):

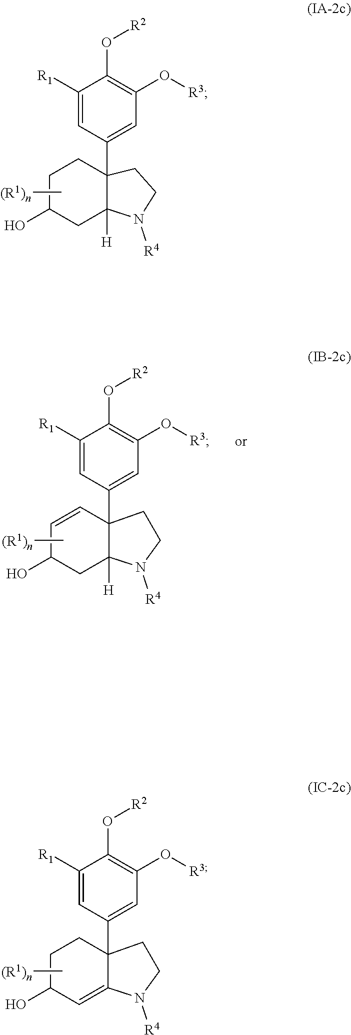

or a pharmaceutically acceptable salt thereof.

In certain embodiments, the compound is of Formula (IA-2) or (IA-2c) or a pharmaceutically acceptable salt thereof.

In certain embodiments, the compound is of Formula (IB-2) or (IB-2c) or a pharmaceutically acceptable salt thereof.

In certain embodiments, the compound is of Formula (IC-2) or (IC-2c) or a pharmaceutically acceptable salt thereof.

In certain embodiments, the compound is of Formula (IA-2a), (IA-2b), (IB-2a), (IB-2b), (IC-2a) or (IC-2b):

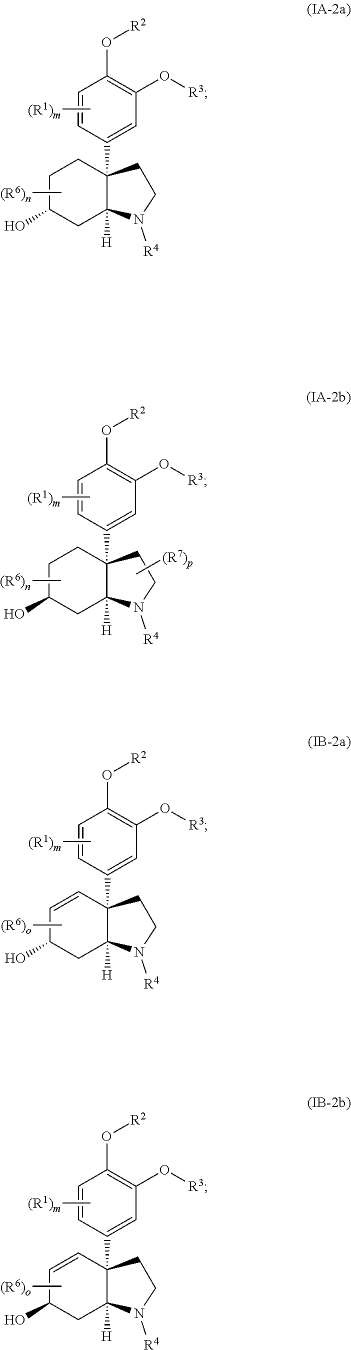

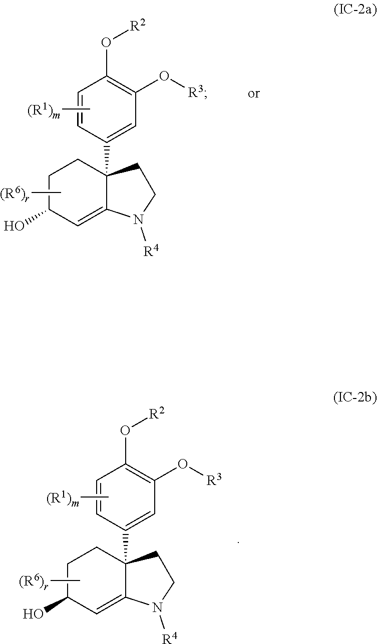

or a pharmaceutically acceptable salt thereof.

In certain embodiments, the compounds of Formula (IA-2a), (IA-2b), (IB-2a), (IB-2b), (IC-2a), and (IC-2b) have the absolute stereochemistry shown.

In certain embodiments, the compound is of Formula (IA-2a) or a pharmaceutically acceptable salt thereof.

In certain embodiments, the compound is of Formula (IA-2b) or a pharmaceutically acceptable salt thereof.

In certain embodiments, the compound is of Formula (IB-2a) or a pharmaceutically acceptable salt thereof.

In certain embodiments, the compound is of Formula (IB-2b) or a pharmaceutically acceptable salt thereof.

In certain embodiments, the compound is of Formula (IC-2a) or a pharmaceutically acceptable salt thereof.

In certain embodiments, the compound is of Formula (IC-2b) or a pharmaceutically acceptable salt thereof.

In certain embodiments, the compound is of Formula (IA-3), (IB-3) or (IC-3):

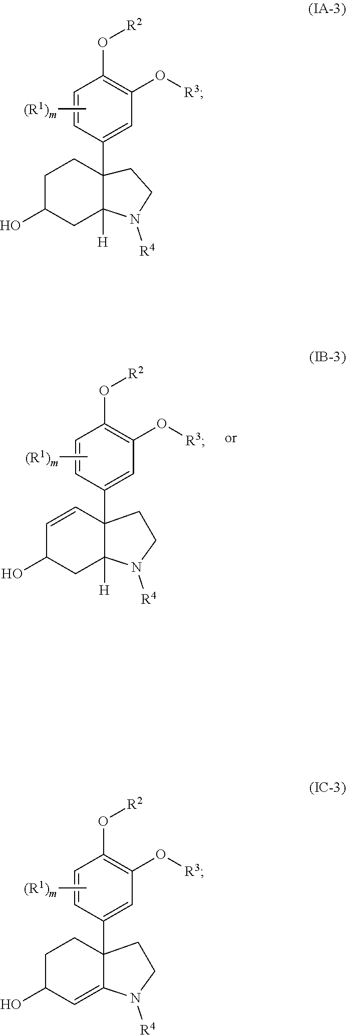

or a pharmaceutically acceptable salt thereof.

In certain embodiments, the compound is of Formula (IA-3) or a pharmaceutically acceptable salt thereof.

In certain embodiments, the compound is of Formula (IB-3) or a pharmaceutically acceptable salt thereof.

In certain embodiments, the compound is of Formula (IC-3) or a pharmaceutically acceptable salt thereof.

In certain embodiments, the compound is of Formula (IA-3a), (IA-3b), (IB-3a), (IB-3b) (IC-3a) or (IC-3b):

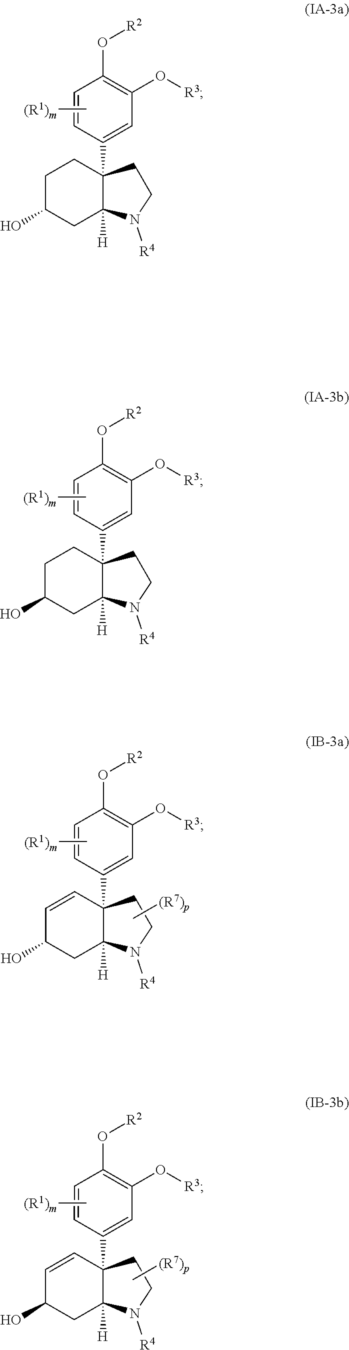

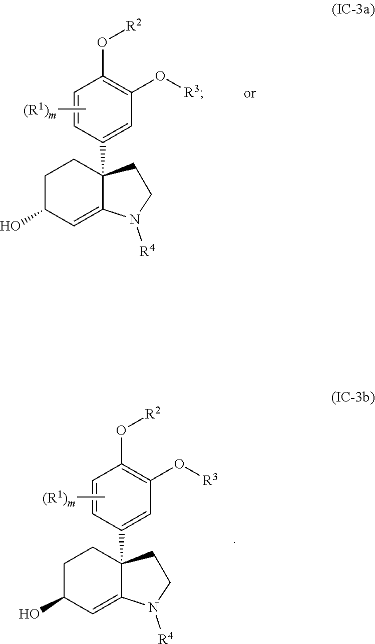

or a pharmaceutically acceptable salt thereof.

In certain embodiments, the compounds of Formula (IA-3a), (IA-3b), (IB-3a), (IB-3b), (IC-3a), and (IC-3b) have the absolute stereochemistry shown.

In certain embodiments, the compound is of Formula (IA-3a) or a pharmaceutically acceptable salt thereof.

In certain embodiments, the compound is of Formula (IA-3b) or a pharmaceutically acceptable salt thereof.

In certain embodiments, the compound is of Formula (IB-3a) or a pharmaceutically acceptable salt thereof.

In certain embodiments, the compound is of Formula (IB-3b) or a pharmaceutically acceptable salt thereof.

In certain embodiments, the compound is of Formula (IC-3a) or a pharmaceutically acceptable salt thereof.

In certain embodiments, the compound is of Formula (IC-3b) or a pharmaceutically acceptable salt thereof.

In certain embodiments, the compound is of Formula (IA-4), (IB-4) or (IC-4):

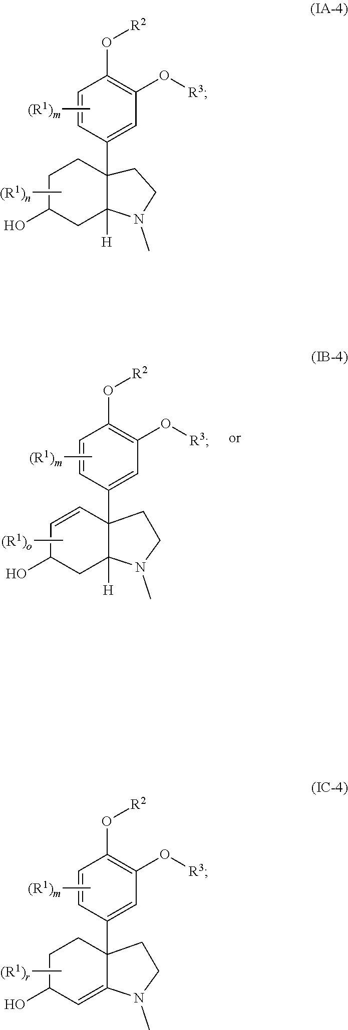

or a pharmaceutically acceptable salt thereof.

In certain embodiments, the compound is of Formula (IA-4) or a pharmaceutically acceptable salt thereof.

In certain embodiments, the compound is of Formula (IB-4) or a pharmaceutically acceptable salt thereof.

In certain embodiments, the compound is of Formula (IC-4) or a pharmaceutically acceptable salt thereof.

In certain embodiments, the compound is of Formula (IA-4a), (IA-4b), (IB-4a), (IB-4b), (IC-4a) or (IC-4b):

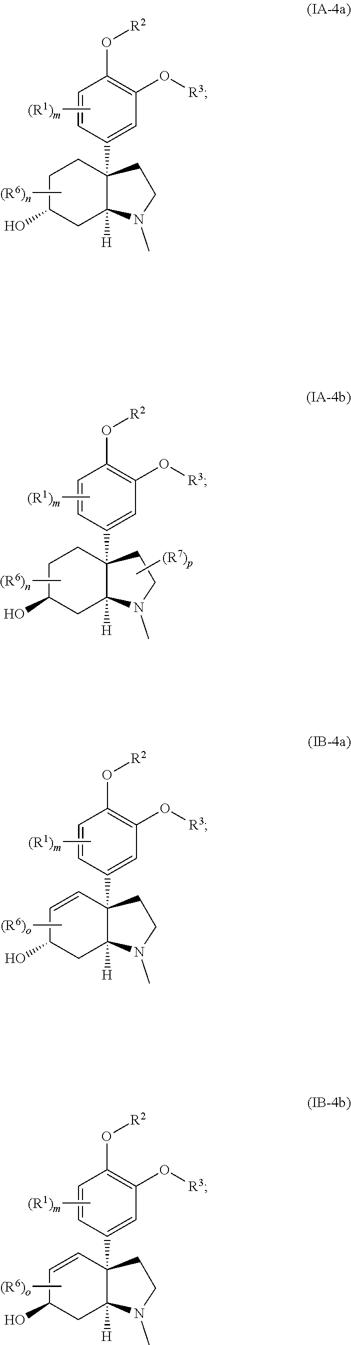

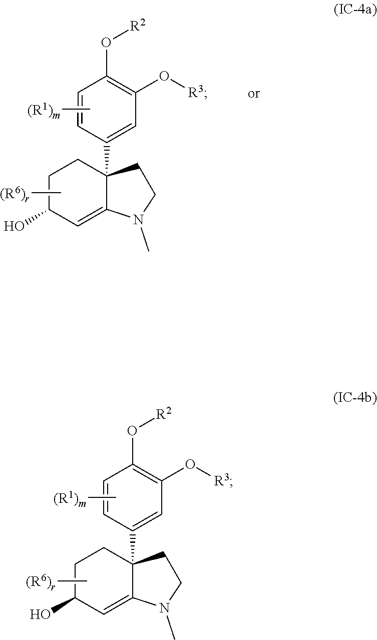

or a pharmaceutically acceptable salt thereof.

In certain embodiments, the compounds of Formula (IA-4a), (IA-4b), (IB-4a), (IB-4b), (IC-4a) and (IC-4b) have the absolute stereochemistry shown.

In certain embodiments, the compound is of Formula (IA-4a) or a pharmaceutically acceptable salt thereof.

In certain embodiments, the compound is of Formula (IA-4b) or a pharmaceutically acceptable salt thereof.

In certain embodiments, the compound is of Formula (IB-4a) or a pharmaceutically acceptable salt thereof.

In certain embodiments, the compound is of Formula (IB-4b) or a pharmaceutically acceptable salt thereof.

In certain embodiments, the compound is of Formula (IC-4a) or a pharmaceutically acceptable salt thereof.

In certain embodiments, the compound is of Formula (IC-4b) or a pharmaceutically acceptable salt thereof.

In certain embodiments, the compound is of Formula (IA-5), (IB-5) or (IC-5):

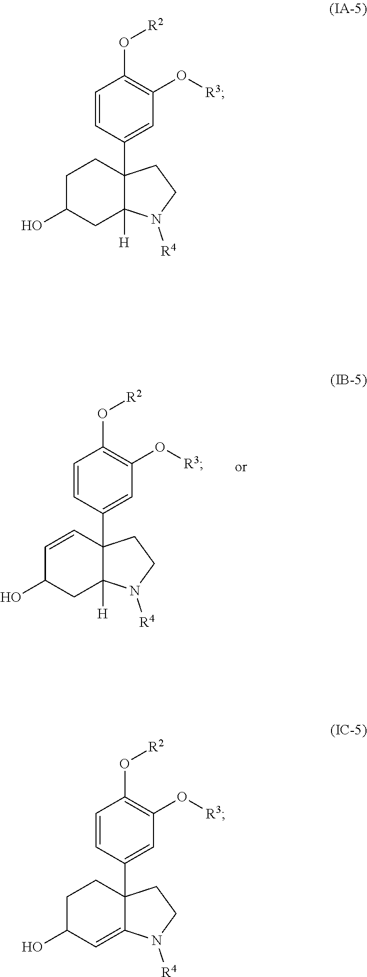

or a pharmaceutically acceptable salt thereof.

In certain embodiments, the compound is of Formula (IA-5) or a pharmaceutically acceptable salt thereof.

In certain embodiments, the compound is of Formula (IB-5) or a pharmaceutically acceptable salt thereof.

In certain embodiments, the compound is of Formula (IC-5) or a pharmaceutically acceptable salt thereof.

In certain embodiments, the compound is of Formula (IA-5a), (IA-5b), (IB-5a), (IB-5b), (IC-5a), or (IC-5b):

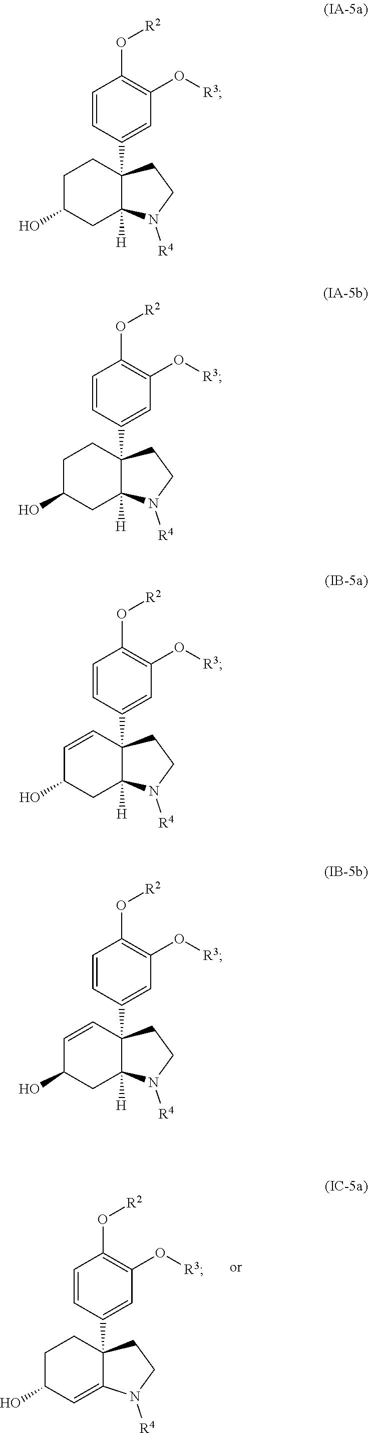

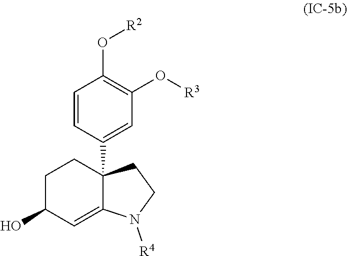

or a pharmaceutically acceptable salt thereof.

In certain embodiments, the compounds of Formula (IA-5a), (IA-5b), (IB-5a), (IB-5b), (IC-5a) and (IC-5b) have the absolute stereochemistry shown.

In certain embodiments, the compound is of Formula (IA-5a) or a pharmaceutically acceptable salt thereof.

In certain embodiments, the compound is of Formula (IA-5b) or a pharmaceutically acceptable salt thereof.

In certain embodiments, the compound is of Formula (IB-5a) or a pharmaceutically acceptable salt thereof.

In certain embodiments, the compound is of Formula (IB-5b) or a pharmaceutically acceptable salt thereof.

In certain embodiments, the compound is of Formula (IC-5a) or a pharmaceutically acceptable salt thereof.

In certain embodiments, the compound is of Formula (IC-5b) or a pharmaceutically acceptable salt thereof.

In certain embodiments, the compound is selected from the group consisting of:

TABLE 3
Compounds of Formula IA-5a, IA-5b, IB-5a, IB-5b, IC-5a and IC-5b

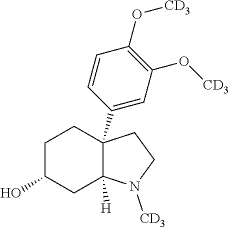 [SMILES: [2H]C([2H])([2H])Oc1ccc([C@@]23CC[C@@H](O)C[C@@H]2N(C([2H])([2H])[2H])CC3)cc1OC([2H])([2H])[2H]]

0501

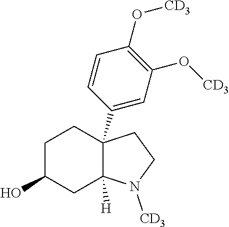 [SMILES: [2H]C([2H])([2H])Oc1ccc([C@@]23CC[C@H](O)C[C@@H]2N(C([2H])([2H])[2H])CC3)cc1OC([2H])([2H])[2H]]

0502

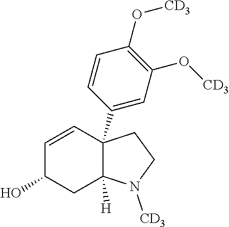 [SMILES: [2H]C([2H])([2H])Oc1ccc([C@@]23C=C[C@@H](O)C[C@@H]2N(C([2H])([2H])[2H])CC3)cc1OC([2H])([2H])[2H]]

0503

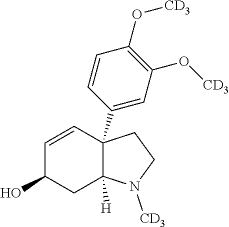 [SMILES: [2H]C([2H])([2H])Oc1ccc([C@@]23C=C[C@H](O)C[C@@H]2N(C([2H])([2H])[2H])CC3)cc1OC([2H])([2H])[2H]]

0504

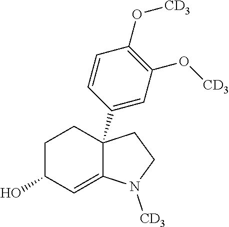 [SMILES: [2H]C([2H])([2H])Oc1ccc([C@@]23CC[C@@H](O)C=C2N(C([2H])([2H])[2H])CC3)cc1OC([2H])([2H])[2H]]

0505

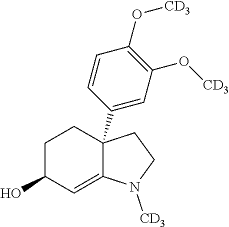 [SMILES: [2H]C([2H])([2H])Oc1ccc([C@@]23CC[C@H](O)C=C2N(C([2H])([2H])[2H])CC3)cc1OC([2H])([2H])[2H]]

0506

or a pharmaceutically acceptable salt thereof.

In certain embodiments, the compound is of Formula (IA-6), (IB-6) or (IC-6):

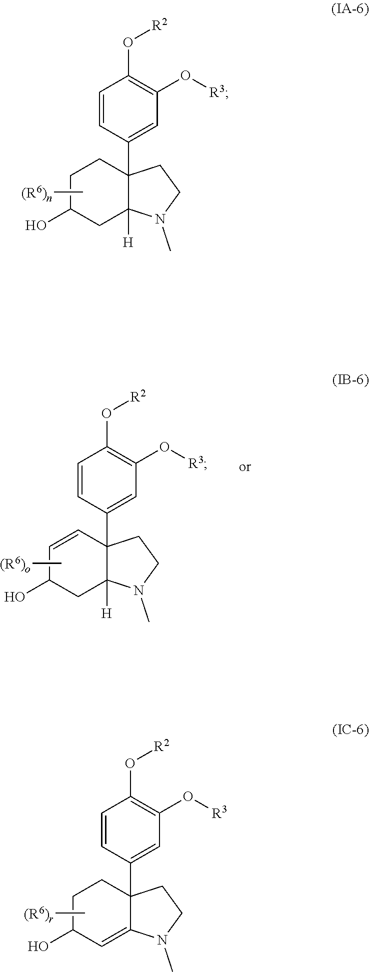

or a pharmaceutically acceptable salt thereof.

In certain embodiments, the compound is of Formula (IA-6) or a pharmaceutically acceptable salt thereof.

In certain embodiments, the compound is of Formula (IB-6) or a pharmaceutically acceptable salt thereof.

In certain embodiments, the compound is of Formula (IC-6) or a pharmaceutically acceptable salt thereof.

In certain embodiments, the compound is of Formula (IA-6a), (IA-6b), (IB-6a), (IB-6b), (IC-6a) or (IC-6b):

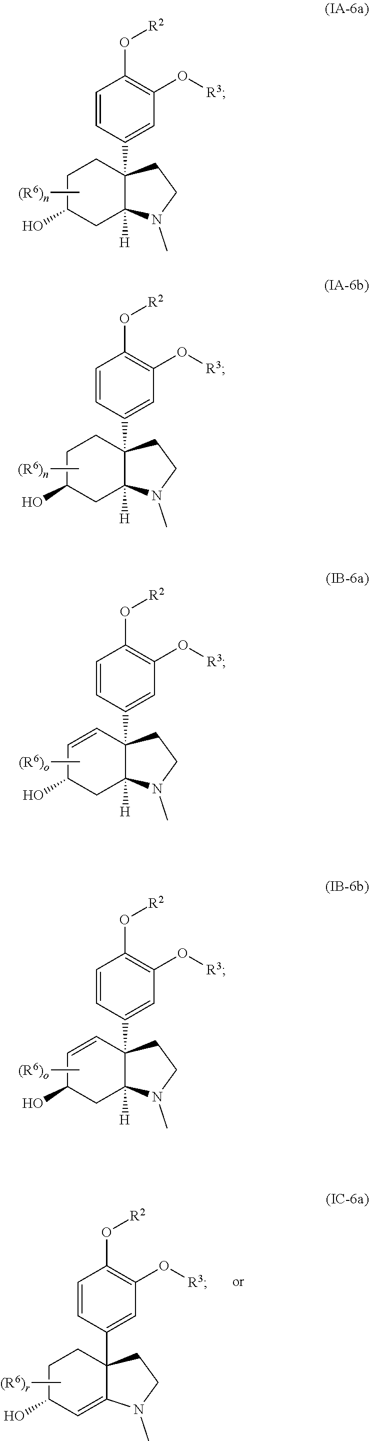

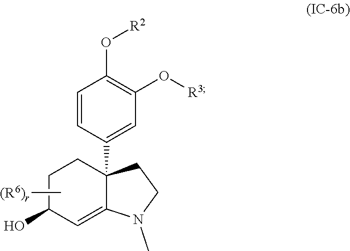

or a pharmaceutically acceptable salt thereof.

In certain embodiments, the compounds of Formula (IA-6a), (IA-6b), (IB-6a), (IB-6b), (IC-6a) and (IC-6b) have the absolute stereochemistry shown.

In certain embodiments, the compound is of Formula (IA-6a) or a pharmaceutically acceptable salt thereof.

In certain embodiments, the compound is of Formula (IA-6b) or a pharmaceutically acceptable salt thereof.

In certain embodiments, the compound is of Formula (IB-6a) or a pharmaceutically acceptable salt thereof.

In certain embodiments, the compound is of Formula (IB-6b) or a pharmaceutically acceptable salt thereof.

In certain embodiments, the compound is of Formula (IC-6a) or a pharmaceutically acceptable salt thereof.

In certain embodiments, the compound is of Formula (IC-6b) or a pharmaceutically acceptable salt thereof.

In certain embodiments, the compound is of Formula (IA-7), (IB-7) or (IC-7):

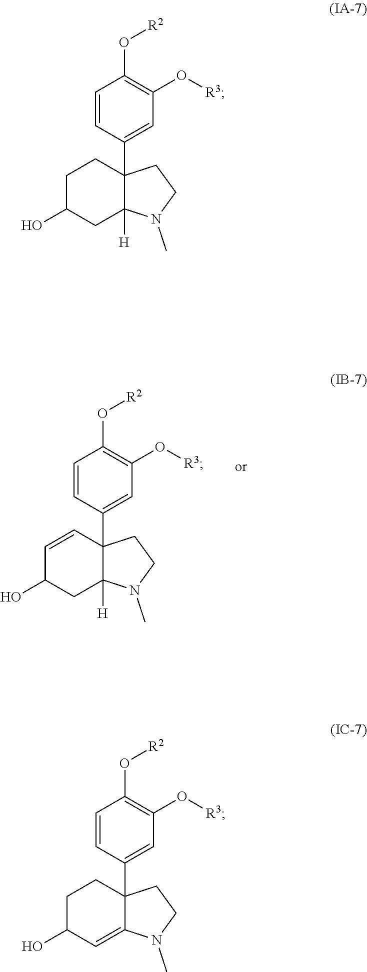

or a pharmaceutically acceptable salt thereof.

In certain embodiments, the compound is of Formula (IA-7) or a pharmaceutically acceptable salt thereof.

In certain embodiments, the compound is of Formula (IB-7) or a pharmaceutically acceptable salt thereof.

In certain embodiments, the compound is of Formula (IC-7) or a pharmaceutically acceptable salt thereof.

In certain embodiments, the compound is of Formula (IA-7a), (IA-7b), (IB-7a), (IB-7b), (IC-7a) or (IC-7b):

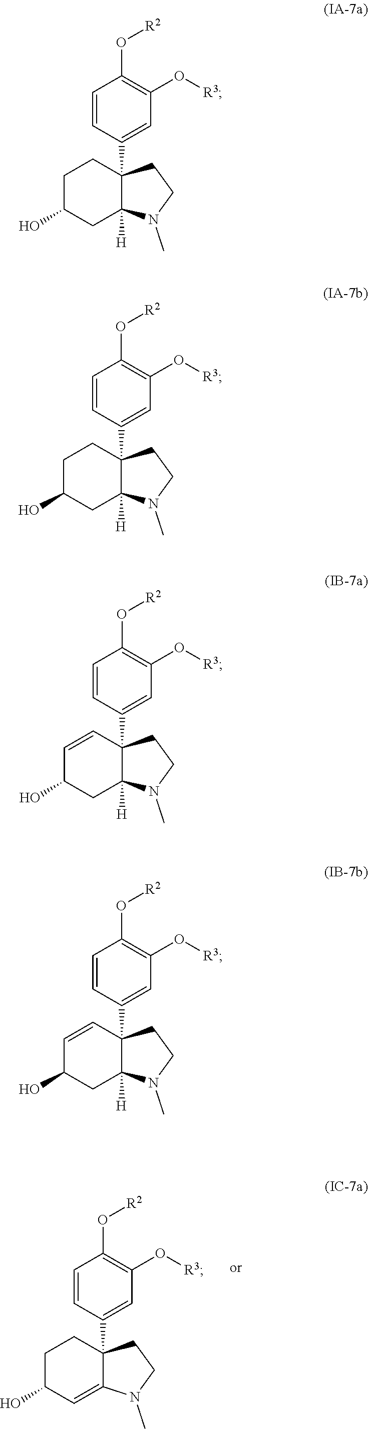

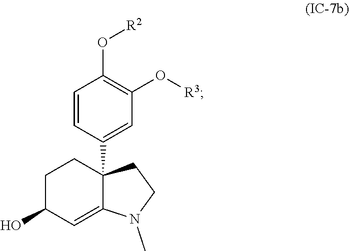

or a pharmaceutically acceptable salt thereof.

In certain embodiments, the compounds of Formula (IA-7a), (IA-7b), (IB-7a), (IB-7b), (IC-7a) and (IC-7b) have the absolute stereochemistry shown.

In certain embodiments, the compound is of Formula (IA-7a) or a pharmaceutically acceptable salt thereof.

In certain embodiments, the compound is of Formula (IA-7b) or a pharmaceutically acceptable salt thereof.

In certain embodiments, the compound is of Formula (IB-7a) or a pharmaceutically acceptable salt thereof.

In certain embodiments, the compound is of Formula (IB-7b) or a pharmaceutically acceptable salt thereof.

In certain embodiments, the compound is of Formula (IC-7a) or a pharmaceutically acceptable salt thereof.

In certain embodiments, the compound is of Formula (IC-7b) or a pharmaceutically acceptable salt thereof.

In certain embodiments, each of R² and R³ is individually H, C₃-C₆ cycloalkyl, C₁-C₆ alkyl optionally substituted by fluoro, deuterium, C₃-C₆ cycloalkyl, C₂-C₆ alkenyl, C₂-C₆ alkynyl or —OC₁-C₆ alkyl. In certain embodiments, R² and R³ together with the atoms to which they are attached combine to form 5 to 7-membered heterocyclyl or 5 to 7-membered heteroaryl, wherein each hydrogen atom in 5 to 7-membered heterocyclyl and 5 to 7-membered heteroaryl is optionally substituted by halo or OR^{a}.

In certain embodiments, the compound is selected from the group consisting of:

TABLE 4
Compounds of Formula IA-7a, IA-7b, IB-7a, IB-7b, IC-7a and IC-7b

| | |
|---|---|
| 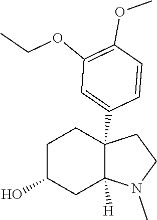 [SMILES: CCOc1cc([C@@]23CC[C@@H](O)C[C@@H]2N(C)CC3)ccc1OC] | 0701 |
| 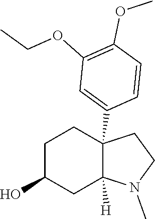 [SMILES: CCOc1cc([C@@]23CC[C@H](O)C[C@@H]2N(C)CC3)ccc1OC] | 0702 |
| 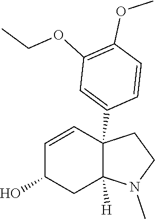 [SMILES: CCOc1cc([C@@]23C=C[C@@H](O)C[C@@H]2N(C)CC3)ccc1OC] | 0703 |
| 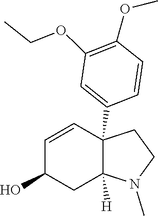 [SMILES: CCOc1cc([C@@]23C=C[C@H](O)C[C@@H]2N(C)CC3)ccc1OC] | 0704 |
| 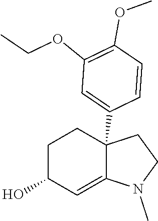 [SMILES: CCOc1cc([C@@]23CC[C@@H](O)C=C2N(C)CC3)ccc1OC] | 0705 |
| 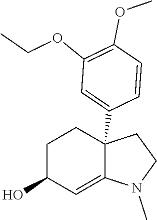 [SMILES: CCOc1cc([C@@]23CC[C@H](O)C=C2N(C)CC3)ccc1OC] | 0706 |
| 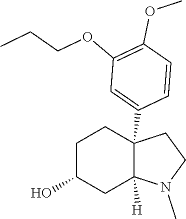 [SMILES: CCCOc1cc([C@@]23CC[C@@H](O)C[C@@H]2N(C)CC3)ccc1OC] | 0707 |
| 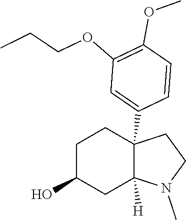 [SMILES: CCCOc1cc([C@@]23CC[C@H](O)C[C@@H]2N(C)CC3)ccc1OC] | 0708 |
| 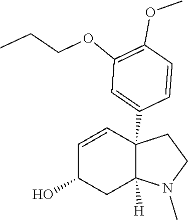 [SMILES: CCCOc1cc([C@@]23C=C[C@@H](O)C[C@@H]2N(C)CC3)ccc1OC] | 0709 |
| 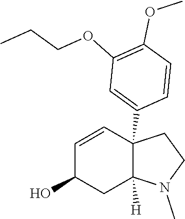 [SMILES: CCCOc1cc([C@@]23C=C[C@H](O)C[C@@H]2N(C)CC3)ccc1OC] | 0710 |
| 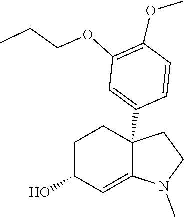 [SMILES: CCCOc1cc([C@@]23CC[C@@H](O)C=C2N(C)CC3)ccc1OC] | 0711 |
| 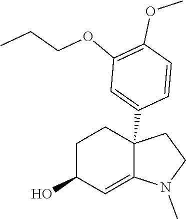 [SMILES: CCCOc1cc([C@@]23CC[C@H](O)C=C2N(C)CC3)ccc1OC] | 0712 |
| 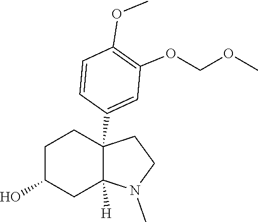 [SMILES: COCOc1cc([C@@]23CC[C@@H](O)C[C@@H]2N(C)CC3)ccc1OC] | 0713 |
| 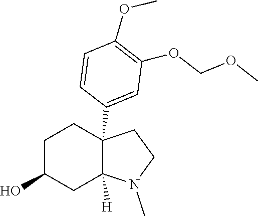 [SMILES: COCOc1cc([C@@]23CC[C@H](O)C[C@@H]2N(C)CC3)ccc1OC] | 0714 |
| 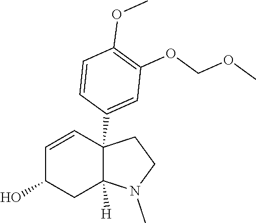 [SMILES: COCOc1cc([C@@]23C=C[C@@H](O)C[C@@H]2N(C)CC3)ccc1OC] | 0715 |
| 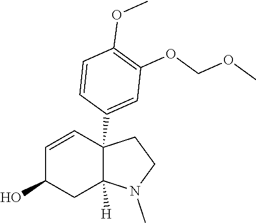 [SMILES: COCOc1cc([C@@]23C=C[C@H](O)C[C@@H]2N(C)CC3)ccc1OC] | 0716 |
| 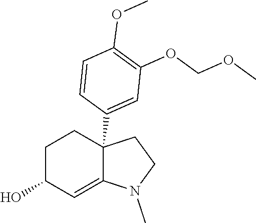 [SMILES: COCOc1cc([C@@]23CC[C@@H](O)C=C2N(C)CC3)ccc1OC] | 0717 |
| 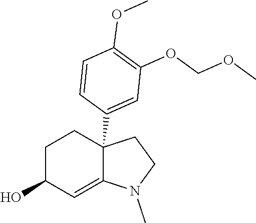 [SMILES: COCOc1cc([C@@]23CC[C@H](O)C=C2N(C)CC3)ccc1OC] | 0718 |
| 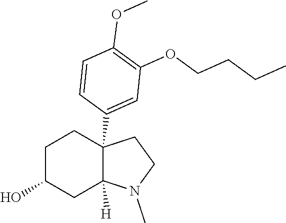 [SMILES: CCCCOc1cc([C@@]23CC[C@@H](O)C[C@@H]2N(C)CC3)ccc1OC] | 0719 |
| 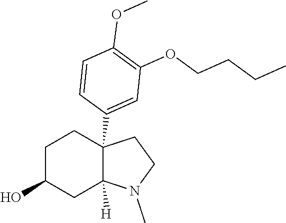 [SMILES: CCCCOc1cc([C@@]23CC[C@H](O)C[C@@H]2N(C)CC3)ccc1OC] | 0720 |
| 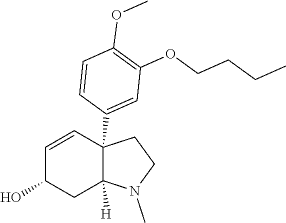 [SMILES: CCCCOc1cc([C@@]23C=C[C@@H](O)C[C@@H]2N(C)CC3)ccc1OC] | 0721 |
| 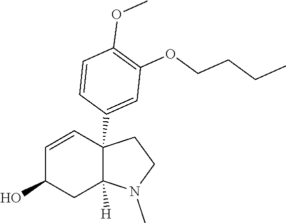 [SMILES: CCCCOc1cc([C@@]23C=C[C@H](O)C[C@@H]2N(C)CC3)ccc1OC] | 0722 |
| 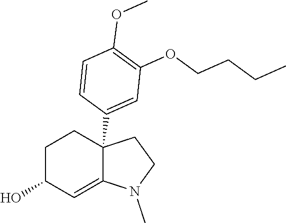 [SMILES: CCCCOc1cc([C@@]23CC[C@@H](O)C=C2N(C)CC3)ccc1OC] | 0723 |
| 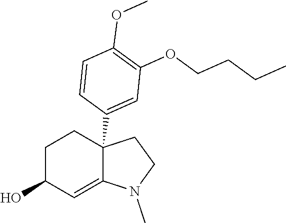 [SMILES: CCCCOc1cc([C@@]23CC[C@H](O)C=C2N(C)CC3)ccc1OC] | 0724 |
| 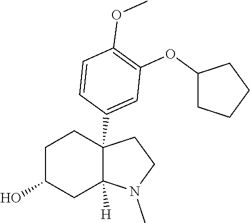 [SMILES: COc1ccc([C@@]23CC[C@@H](O)C[C@@H]2N(C)CC3)cc1OC1CCCC1] | 0725 |
| 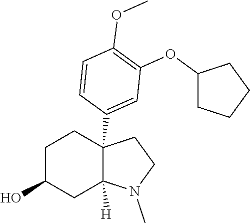 [SMILES: COc1ccc([C@@]23CC[C@H](O)C[C@@H]2N(C)CC3)cc1OC1CCCC1] | 0726 |
| 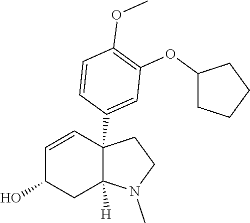 [SMILES: COc1ccc([C@@]23C=C[C@@H](O)C[C@@H]2N(C)CC3)cc1OC1CCCC1] | 0727 |
| 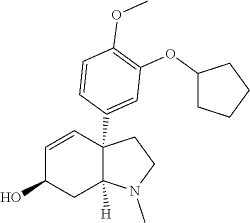 [SMILES: COc1ccc([C@@]23C=C[C@H](O)C[C@@H]2N(C)CC3)cc1OC1CCCC1] | 0728 |
| 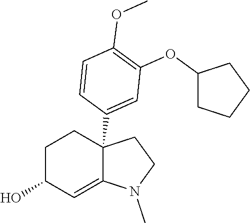 [SMILES: COc1ccc([C@@]23CC[C@@H](O)C=C2N(C)CC3)cc1OC1CCCC1] | 0729 |
| 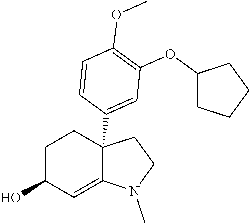 [SMILES: COc1ccc([C@@]23CC[C@H](O)C=C2N(C)CC3)cc1OC1CCCC1] | 0730 |
| 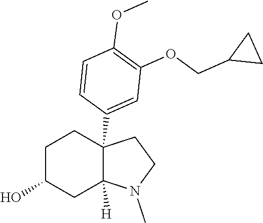 [SMILES: COc1ccc([C@@]23CC[C@@H](O)C[C@@H]2N(C)CC3)cc1OCC1CC1] | 0731 |
| 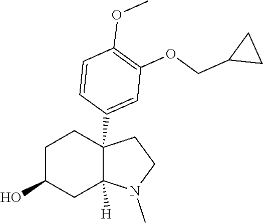 [SMILES: COc1ccc([C@@]23CC[C@H](O)C[C@@H]2N(C)CC3)cc1OCC1CC1] | 0732 |
| 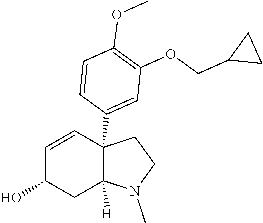 [SMILES: COc1ccc([C@@]23C=C[C@@H](O)C[C@@H]2N(C)CC3)cc1OCC1CC1] | 0733 |
| 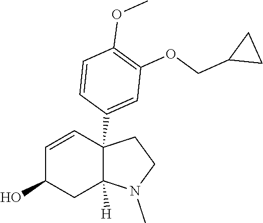 [SMILES: COc1ccc([C@@]23C=C[C@H](O)C[C@@H]2N(C)CC3)cc1OCC1CC1] | 0734 |
| 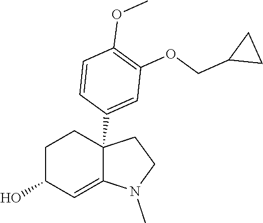 [SMILES: COc1ccc([C@@]23CC[C@@H](O)C=C2N(C)CC3)cc1OCC1CC1] | 0735 |
| 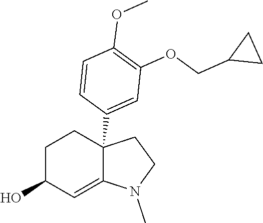 [SMILES: COc1ccc([C@@]23CC[C@H](O)C=C2N(C)CC3)cc1OCC1CC1] | 0736 |
| 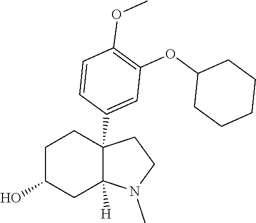 [SMILES: COc1ccc([C@@]23CC[C@@H](O)C[C@@H]2N(C)CC3)cc1OC1CCCCC1] | 0737 |
| 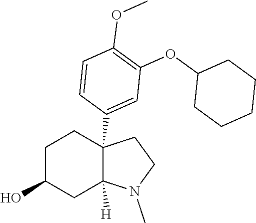 [SMILES: COc1ccc([C@@]23CC[C@H](O)C[C@@H]2N(C)CC3)cc1OC1CCCCC1] | 0738 |
| 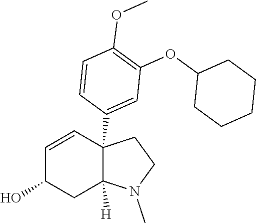 [SMILES: COc1ccc([C@@]23C=C[C@@H](O)C[C@@H]2N(C)CC3)cc1OC1CCCCC1] | 0739 |
| 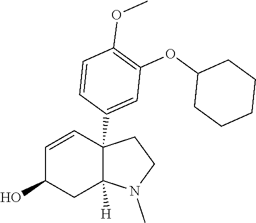 [SMILES: COc1ccc([C@@]23C=C[C@H](O)C[C@@H]2N(C)CC3)cc1OC1CCCCC1] | 0740 |
| 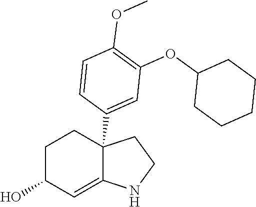 [SMILES: COc1ccc([C@]23CCNC2=C[C@H](O)CC3)cc1OC1CCCCC1] | 0741 |
| 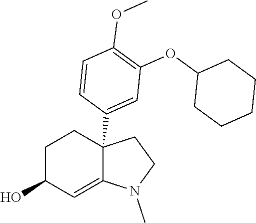 [SMILES: COc1ccc([C@@]23CC[C@H](O)C=C2N(C)CC3)cc1OC1CCCCC1] | 0742 |
| 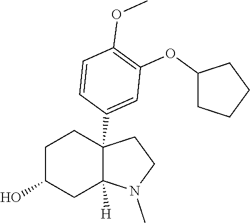 [SMILES: COc1ccc([C@@]23CC[C@@H](O)C[C@@H]2N(C)CC3)cc1OC1CCCC1] | 0743 |
| 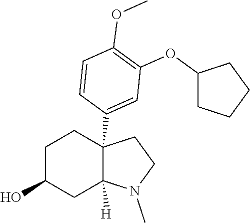 [SMILES: COc1ccc([C@@]23CC[C@H](O)C[C@@H]2N(C)CC3)cc1OC1CCCC1] | 0744 |
| 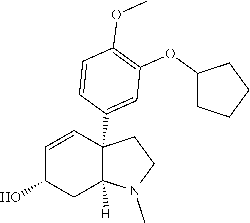 [SMILES: COc1ccc([C@@]23C=C[C@@H](O)C[C@@H]2N(C)CC3)cc1OC1CCCC1] | 0745 |
| 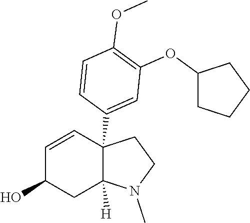 [SMILES: COc1ccc([C@@]23C=C[C@H](O)C[C@@H]2N(C)CC3)cc1OC1CCCC1] | 0746 |
| 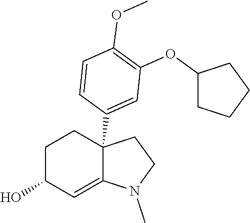 [SMILES: COc1ccc([C@@]23CC[C@@H](O)C=C2N(C)CC3)cc1OC1CCCC1] | 0747 |
| 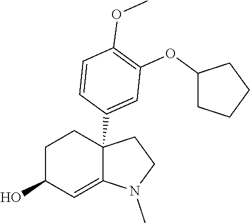 [SMILES: COc1ccc([C@@]23CC[C@H](O)C=C2N(C)CC3)cc1OC1CCCC1] | 0748 |
| 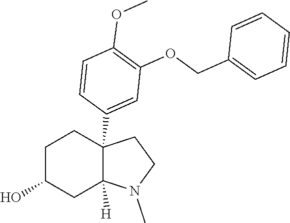 [SMILES: COc1ccc([C@@]23CC[C@@H](O)C[C@@H]2N(C)CC3)cc1OCc1ccccc1] | 0749 |
| 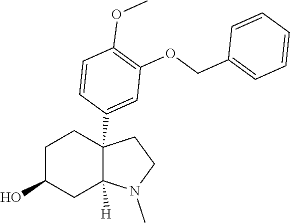 [SMILES: COc1ccc([C@@]23CC[C@H](O)C[C@@H]2N(C)CC3)cc1OCc1ccccc1] | 0750 |
| 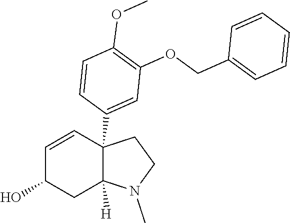 [SMILES: COc1ccc([C@@]23C=C[C@@H](O)C[C@@H]2N(C)CC3)cc1OCc1ccccc1] | 0751 |
| 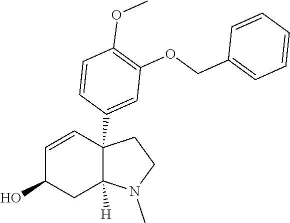 [SMILES: COc1ccc([C@@]23C=C[C@H](O)C[C@@H]2N(C)CC3)cc1OCc1ccccc1] | 0752 |
| 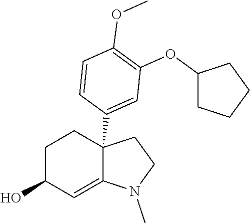 [SMILES: COc1ccc([C@@]23CC[C@H](O)C=C2N(C)CC3)cc1OC1CCCC1] | 0753 |
| 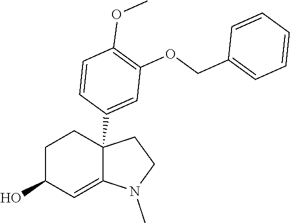 [SMILES: COc1ccc([C@@]23CC[C@H](O)C=C2N(C)CC3)cc1OCc1ccccc1] | 0754 |
| 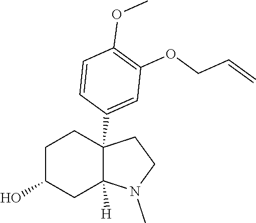 [SMILES: C=CCOc1cc([C@@]23CC[C@@H](O)C[C@@H]2N(C)CC3)ccc1OC] | 0755 |
| 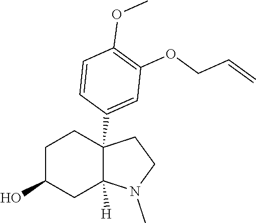 [SMILES: C=CCOc1cc([C@@]23CC[C@H](O)C[C@@H]2N(C)CC3)ccc1OC] | 0756 |
| 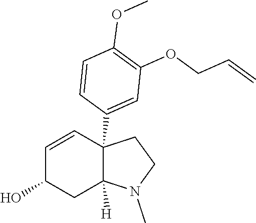 [SMILES: C=CCOc1cc([C@@]23C=C[C@@H](O)C[C@@H]2N(C)CC3)ccc1OC] | 0757 |
| 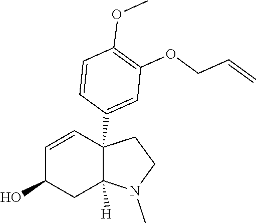 [SMILES: C=CCOc1cc([C@@]23C=C[C@H](O)C[C@@H]2N(C)CC3)ccc1OC] | 0758 |
| 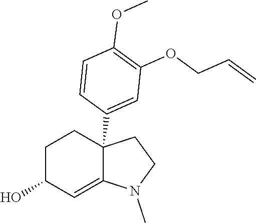 [SMILES: C=CCOc1cc([C@@]23CC[C@@H](O)C=C2N(C)CC3)ccc1OC] | 0759 |
| 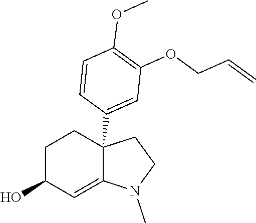 [SMILES: C=CCOc1cc([C@@]23CC[C@H](O)C=C2N(C)CC3)ccc1OC] | 0760 |
| 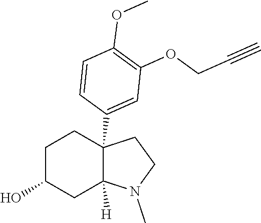 [SMILES: C#CCOc1cc([C@@]23CC[C@@H](O)C[C@@H]2N(C)CC3)ccc1OC] | 0761 |
| 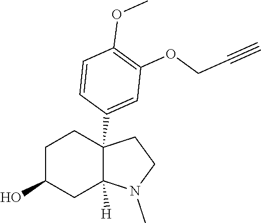 [SMILES: C#CCOc1cc([C@@]23CC[C@H](O)C[C@@H]2N(C)CC3)ccc1OC] | 0762 |
| 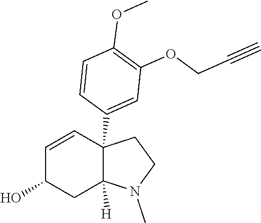 [SMILES: C#CCOc1cc([C@@]23C=C[C@@H](O)C[C@@H]2N(C)CC3)ccc1OC] | 0763 |
| 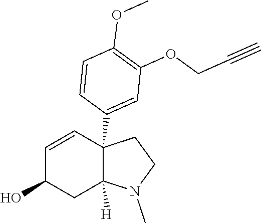 [SMILES: C#CCOc1cc([C@@]23C=C[C@H](O)C[C@@H]2N(C)CC3)ccc1OC] | 0764 |
| 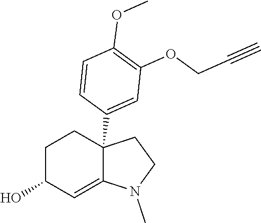 [SMILES: C#CCOc1cc([C@@]23CC[C@@H](O)C=C2N(C)CC3)ccc1OC] | 0765 |
| 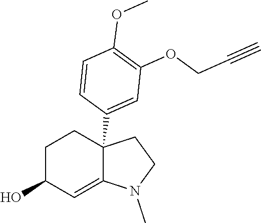 [SMILES: C#CCOc1cc([C@@]23CC[C@H](O)C=C2N(C)CC3)ccc1OC] | 0766 |
| 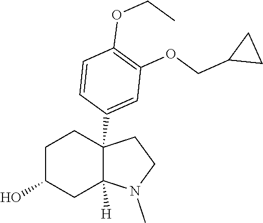 [SMILES: CCOc1ccc([C@@]23CC[C@@H](O)C[C@@H]2N(C)CC3)cc1OCC1CC1] | 0767 |
| 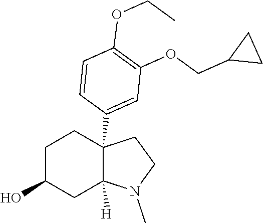 [SMILES: CCOc1ccc([C@@]23CC[C@H](O)C[C@@H]2N(C)CC3)cc1OCC1CC1] | 0768 |
| 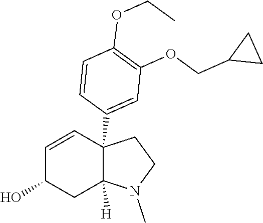 [SMILES: CCOc1ccc([C@@]23C=C[C@@H](O)C[C@@H]2N(C)CC3)cc1OCC1CC1] | 0769 |
| 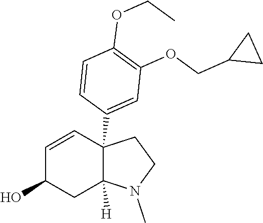 [SMILES: CCOc1ccc([C@@]23C=C[C@H](O)C[C@@H]2N(C)CC3)cc1OCC1CC1] | 0770 |
| 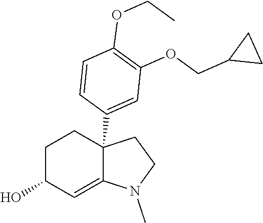 [SMILES: CCOc1ccc([C@@]23CC[C@@H](O)C=C2N(C)CC3)cc1OCC1CC1] | 0771 |
| 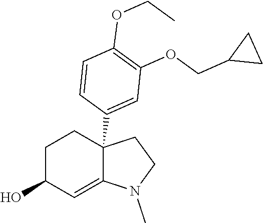 [SMILES: CCOc1ccc([C@@]23CC[C@H](O)C=C2N(C)CC3)cc1OCC1CC1] | 0772 |
| 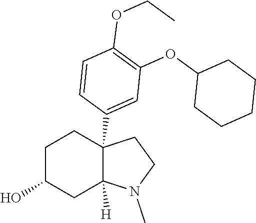 [SMILES: CCOc1ccc([C@@]23CC[C@@H](O)C[C@@H]2N(C)CC3)cc1OC1CCCCC1] | 0773 |
| 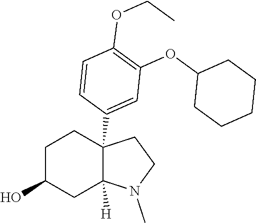 [SMILES: CCOc1ccc([C@@]23CC[C@H](O)C[C@@H]2N(C)CC3)cc1OC1CCCCC1] | 0774 |
| 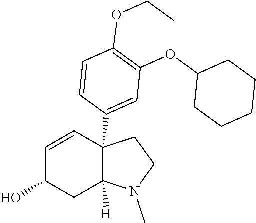 [SMILES: CCOc1ccc([C@@]23C=C[C@@H](O)C[C@@H]2N(C)CC3)cc1OC1CCCCC1] | 0775 |
| 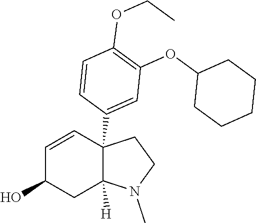 [SMILES: CCOc1ccc([C@@]23C=C[C@H](O)C[C@@H]2N(C)CC3)cc1OC1CCCCC1] | 0776 |
| 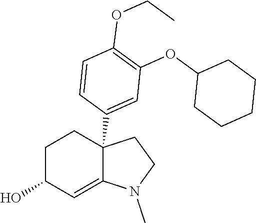 [SMILES: CCOc1ccc([C@@]23CC[C@@H](O)C=C2N(C)CC3)cc1OC1CCCCC1] | 0777 |
| 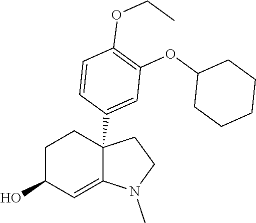 [SMILES: CCOc1ccc([C@@]23CC[C@H](O)C=C2N(C)CC3)cc1OC1CCCCC1] | 0778 |
| 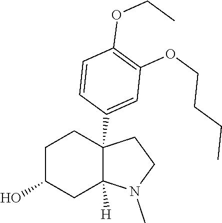 [SMILES: CCCCOc1cc([C@@]23CC[C@@H](O)C[C@@H]2N(C)CC3)ccc1OCC] | 0779 |
| 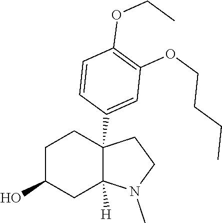 [SMILES: CCCCOc1cc([C@@]23CC[C@H](O)C[C@@H]2N(C)CC3)ccc1OCC] | 0780 |
| 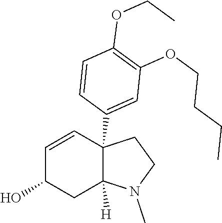 [SMILES: CCCCOc1cc([C@@]23C=C[C@@H](O)C[C@@H]2N(C)CC3)ccc1OCC] | 0781 |
| 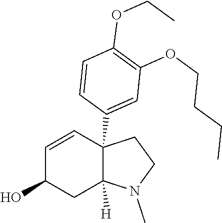 [SMILES: CCCCOc1cc([C@@]23C=C[C@H](O)C[C@@H]2N(C)CC3)ccc1OCC] | 0782 |
| 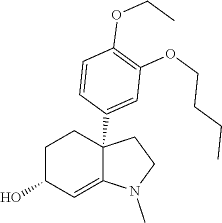 [SMILES: CCCCOc1cc([C@@]23CC[C@@H](O)C=C2N(C)CC3)ccc1OCC] | 0783 |
| 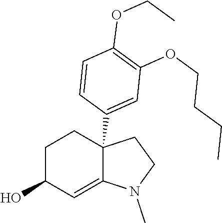 [SMILES: CCCCOc1cc([C@@]23CC[C@H](O)C=C2N(C)CC3)ccc1OCC] | 0784 |
| 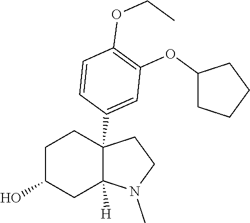 [SMILES: CCOc1ccc([C@@]23CC[C@@H](O)C[C@@H]2N(C)CC3)cc1OC1CCCC1] | 0785 |
| 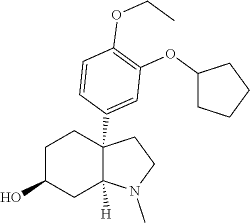 [SMILES: CCOc1ccc([C@@]23CC[C@H](O)C[C@@H]2N(C)CC3)cc1OC1CCCC1] | 0786 |
| 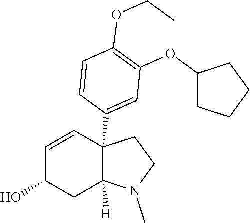 [SMILES: CCOc1ccc([C@@]23C=C[C@@H](O)C[C@@H]2N(C)CC3)cc1OC1CCCC1] | 0787 |
| 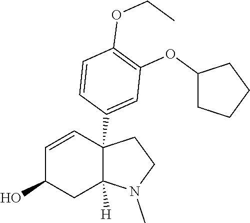 [SMILES: CCOc1ccc([C@@]23C=C[C@H](O)C[C@@H]2N(C)CC3)cc1OC1CCCC1] | 0788 |
| 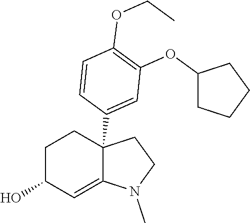 [SMILES: CCOc1ccc([C@@]23CC[C@@H](O)C=C2N(C)CC3)cc1OC1CCCC1] | 0789 |
| 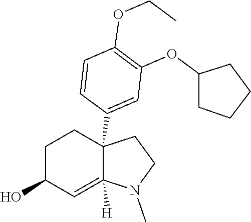 | 0790 |
| 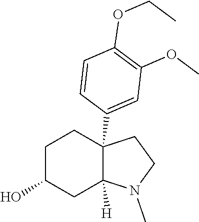 [SMILES: CCOc1ccc([C@@]23CC[C@@H](O)C[C@@H]2N(C)CC3)cc1OC] | 0791 |
| 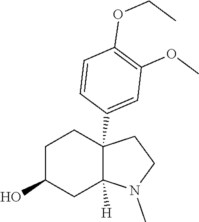 [SMILES: CCOc1ccc([C@@]23CC[C@H](O)C[C@@H]2N(C)CC3)cc1OC] | 0792 |
| 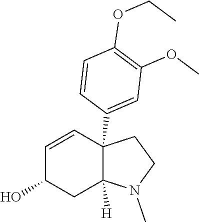 [SMILES: CCOc1ccc([C@@]23C=C[C@@H](O)C[C@@H]2N(C)CC3)cc1OC] | 0793 |
| 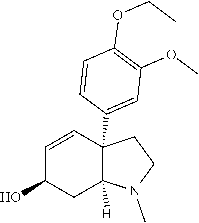 [SMILES: CCOc1ccc([C@@]23C=C[C@H](O)C[C@@H]2N(C)CC3)cc1OC] | 0794 |
| 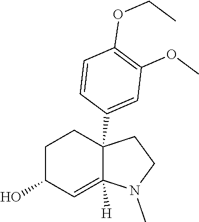 | 0795 |
| 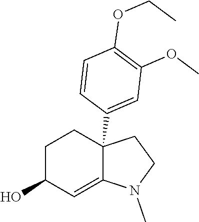 [SMILES: CCOc1ccc([C@@]23CC[C@H](O)C=C2N(C)CC3)cc1OC] | 796 |
| 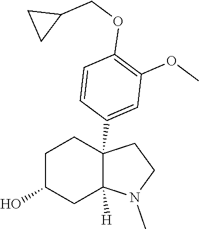 [SMILES: COc1cc([C@@]23CC[C@@H](O)C[C@@H]2N(C)CC3)ccc1OCC1CC1] | 797 |
| 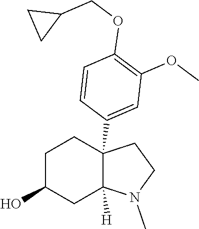 [SMILES: COc1cc([C@@]23CC[C@H](O)C[C@@H]2N(C)CC3)ccc1OCC1CC1] | 798 |
| 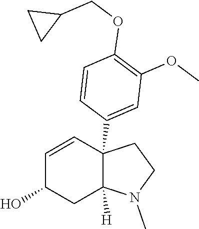 [SMILES: COc1cc([C@@]23C=C[C@@H](O)C[C@@H]2N(C)CC3)ccc1OCC1CC1] | 799 |
| 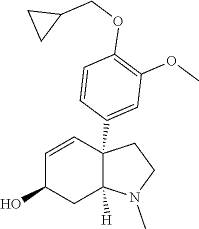 [SMILES: COc1cc([C@@]23C=C[C@H](O)C[C@@H]2N(C)CC3)ccc1OCC1CC1] | 1700 |
| 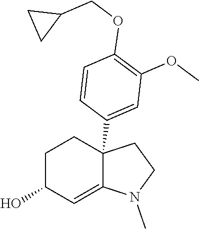 [SMILES: COc1cc([C@@]23CC[C@@H](O)C=C2N(C)CC3)ccc1OCC1CC1] | 1701 |
| 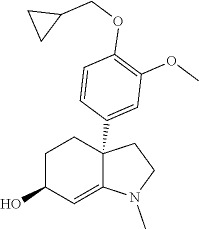 [SMILES: COc1cc([C@@]23CC[C@H](O)C=C2N(C)CC3)ccc1OCC1CC1] | 1702 |
| 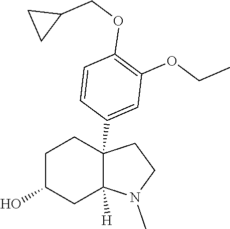 [SMILES: CCOc1cc([C@@]23CC[C@@H](O)C[C@@H]2N(C)CC3)ccc1OCC1CC1] | 1703 |
| 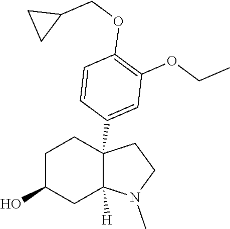 [SMILES: CCOc1cc([C@@]23CC[C@H](O)C[C@@H]2N(C)CC3)ccc1OCC1CC1] | 1704 |
| 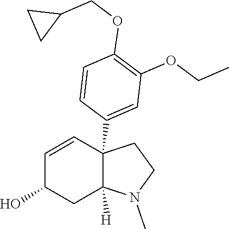 [SMILES: CCOc1cc([C@@]23C=C[C@@H](O)C[C@@H]2N(C)CC3)ccc1OCC1CC1] | 1705 |
| 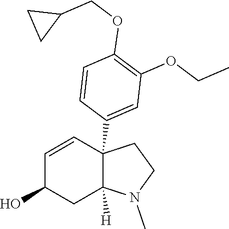 [SMILES: CCOc1cc([C@@]23C=C[C@H](O)C[C@@H]2N(C)CC3)ccc1OCC1CC1] | 1706 |
| 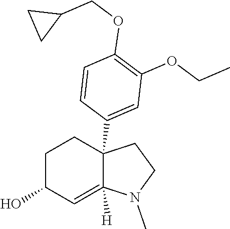 | 1707 |
| 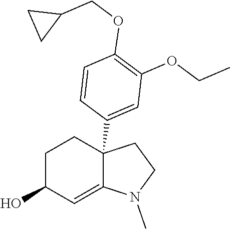 [SMILES: CCOc1cc([C@@]23CC[C@H](O)C=C2N(C)CC3)ccc1OCC1CC1] | 1708 |
| 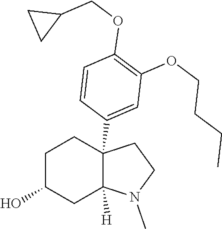 [SMILES: CCCCOc1cc([C@@]23CC[C@@H](O)C[C@@H]2N(C)CC3)ccc1OCC1CC1] | 1709 |
| 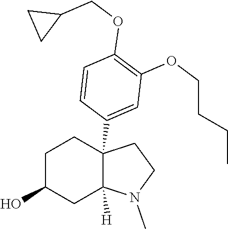 [SMILES: CCCCOc1cc([C@@]23CC[C@H](O)C[C@@H]2N(C)CC3)ccc1OCC1CC1] | 1710 |
| 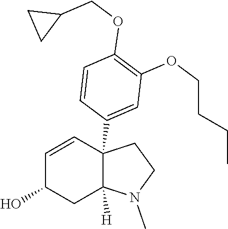 [SMILES: CCCCOc1cc([C@@]23C=C[C@@H](O)C[C@@H]2N(C)CC3)ccc1OCC1CC1] | 1711 |
| 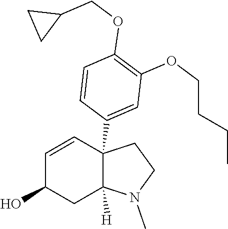 [SMILES: CCCCOc1cc([C@@]23C=C[C@H](O)C[C@@H]2N(C)CC3)ccc1OCC1CC1] | 1712 |
| 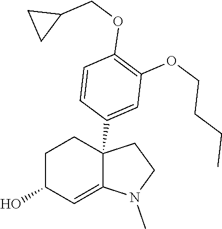 [SMILES: CCCCOc1cc([C@@]23CC[C@@H](O)C=C2N(C)CC3)ccc1OCC1CC1] | 1713 |
| 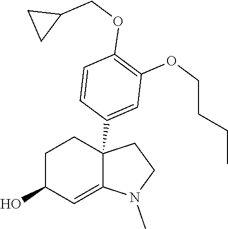 [SMILES: CCCCOc1cc([C@@]23CC[C@H](O)C=C2N(C)CC3)ccc1OCC1CC1] | 1714 |
| 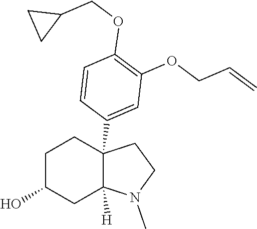 [SMILES: C=CCOc1cc([C@@]23CC[C@@H](O)C[C@@H]2N(C)CC3)ccc1OCC1CC1] | 1715 |
| 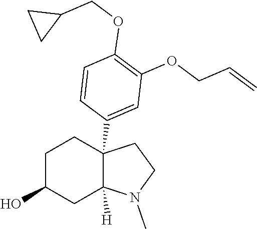 [SMILES: C=CCOc1cc([C@@]23CC[C@H](O)C[C@@H]2N(C)CC3)ccc1OCC1CC1] | 1716 |
| 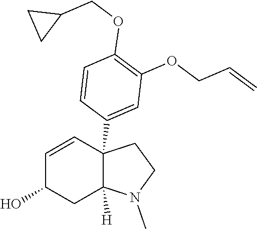 [SMILES: C=CCOc1cc([C@@]23C=C[C@@H](O)C[C@@H]2N(C)CC3)ccc1OCC1CC1] | 1717 |
| 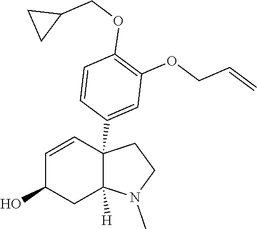 [SMILES: C=CCOc1cc([C@@]23C=C[C@H](O)C[C@@H]2N(C)CC3)ccc1OCC1CC1] | 1718 |
| 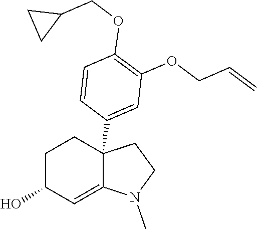 [SMILES: C=CCOc1cc([C@@]23CC[C@@H](O)C=C2N(C)CC3)ccc1OCC1CC1] | 1719 |
| 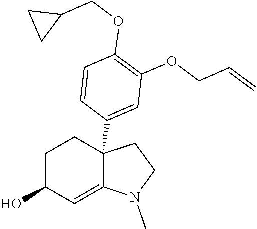 [SMILES: C=CCOc1cc([C@@]23CC[C@H](O)C=C2N(C)CC3)ccc1OCC1CC1] | 1720 |
| 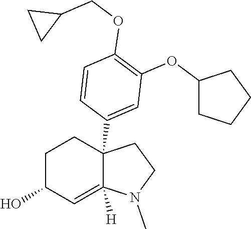 | 1721 |
| 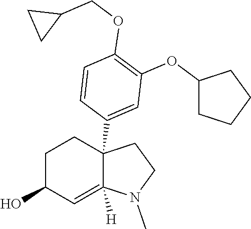 | 1722 |
| 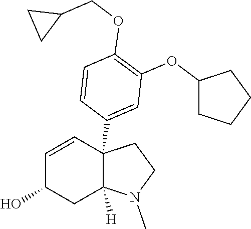 [SMILES: CN1CC[C@]2(c3ccc(OCC4CC4)c(OC4CCCC4)c3)C=C[C@@H](O)C[C@H]12] | 1723 |
| 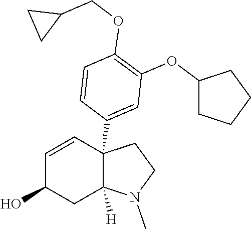 [SMILES: CN1CC[C@]2(c3ccc(OCC4CC4)c(OC4CCCC4)c3)C=C[C@H](O)C[C@H]12] | 1724 |
| 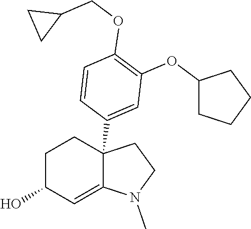 [SMILES: CN1CC[C@]2(c3ccc(OCC4CC4)c(OC4CCCC4)c3)CC[C@@H](O)C=C12] | 1725 |
| 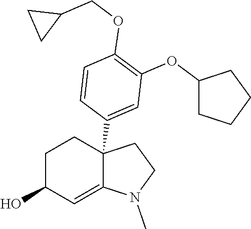 [SMILES: CN1CC[C@]2(c3ccc(OCC4CC4)c(OC4CCCC4)c3)CC[C@H](O)C=C12] | 1726 |
| 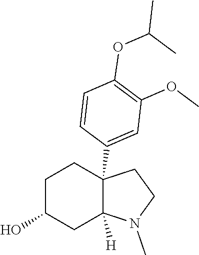 [SMILES: COc1cc([C@@]23CC[C@@H](O)C[C@@H]2N(C)CC3)ccc1OC(C)C] | 1727 |
| 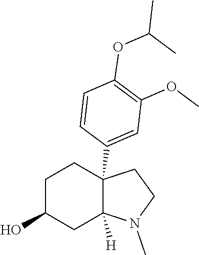 [SMILES: COc1cc([C@@]23CC[C@H](O)C[C@@H]2N(C)CC3)ccc1OC(C)C] | 1728 |
| 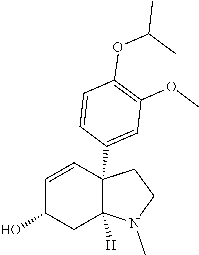 [SMILES: COc1cc([C@@]23C=C[C@@H](O)C[C@@H]2N(C)CC3)ccc1OC(C)C] | 1729 |
| 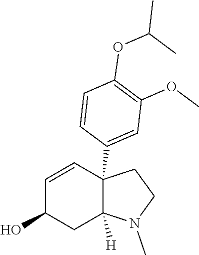 [SMILES: COc1cc([C@@]23C=C[C@H](O)C[C@@H]2N(C)CC3)ccc1OC(C)C] | 1730 |
| 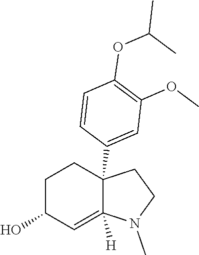 | 1731 |
| 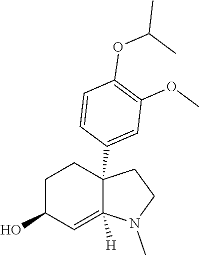 | 1732 |
| 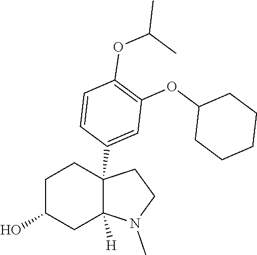 [SMILES: CC(C)Oc1ccc([C@@]23CC[C@@H](O)C[C@@H]2N(C)CC3)cc1OC1CCCCC1] | 1733 |
| 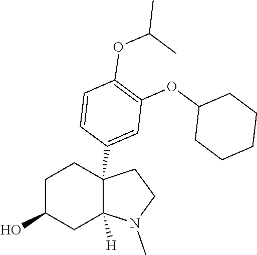 [SMILES: CC(C)Oc1ccc([C@@]23CC[C@H](O)C[C@@H]2N(C)CC3)cc1OC1CCCCC1] | 1734 |
| 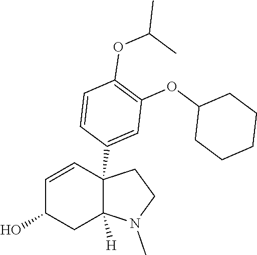 [SMILES: CC(C)Oc1ccc([C@@]23C=C[C@@H](O)C[C@@H]2N(C)CC3)cc1OC1CCCCC1] | 1735 |
| 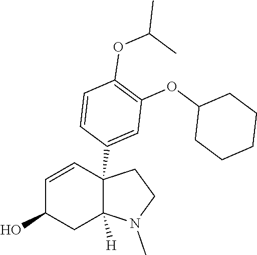 [SMILES: CC(C)Oc1ccc([C@@]23C=C[C@H](O)C[C@@H]2N(C)CC3)cc1OC1CCCCC1] | 1736 |
| 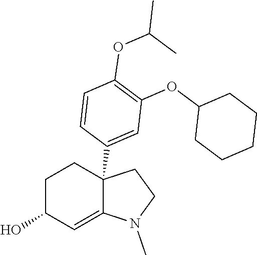 [SMILES: CC(C)Oc1ccc([C@@]23CC[C@@H](O)C=C2N(C)CC3)cc1OC1CCCCC1] | 1737 |
| 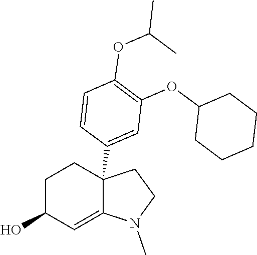 [SMILES: CC(C)Oc1ccc([C@@]23CC[C@H](O)C=C2N(C)CC3)cc1OC1CCCCC1] | 1738 |
| 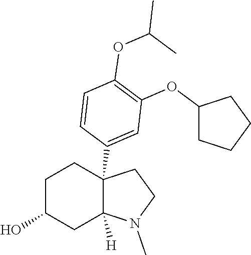 [SMILES: CC(C)Oc1ccc([C@@]23CC[C@@H](O)C[C@@H]2N(C)CC3)cc1OC1CCCC1] | 1739 |
| 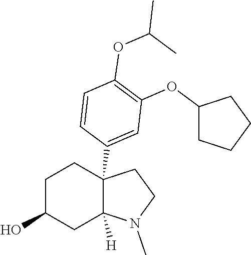 [SMILES: CC(C)Oc1ccc([C@@]23CC[C@H](O)C[C@@H]2N(C)CC3)cc1OC1CCCC1] | 1740 |
| 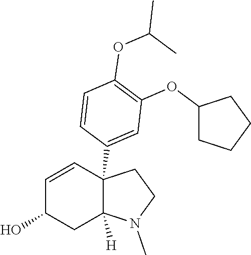 [SMILES: CC(C)Oc1ccc([C@@]23C=C[C@@H](O)C[C@@H]2N(C)CC3)cc1OC1CCCC1] | 1741 |
| 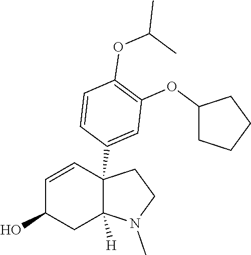 [SMILES: CC(C)Oc1ccc([C@@]23C=C[C@H](O)C[C@@H]2N(C)CC3)cc1OC1CCCC1] | 1742 |
| 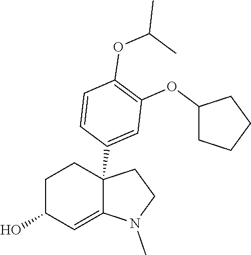 [SMILES: CC(C)Oc1ccc([C@@]23CC[C@@H](O)C=C2N(C)CC3)cc1OC1CCCC1] | 1743 |
| 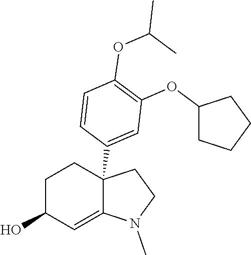 [SMILES: CC(C)Oc1ccc([C@@]23CC[C@H](O)C=C2N(C)CC3)cc1OC1CCCC1] | 1744 |
| 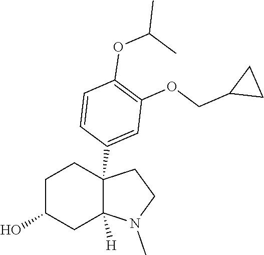 [SMILES: CC(C)Oc1ccc([C@@]23CC[C@@H](O)C[C@@H]2N(C)CC3)cc1OCC1CC1] | 1745 |
| 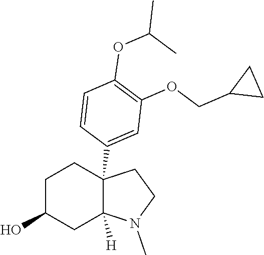 [SMILES: CC(C)Oc1ccc([C@@]23CC[C@H](O)C[C@@H]2N(C)CC3)cc1OCC1CC1] | 1746 |
| 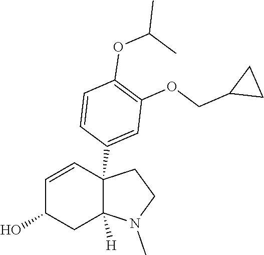 [SMILES: CC(C)Oc1ccc([C@@]23C=C[C@@H](O)C[C@@H]2N(C)CC3)cc1OCC1CC1] | 1747 |
| 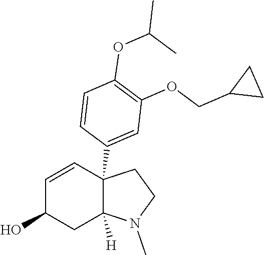 [SMILES: CC(C)Oc1ccc([C@@]23C=C[C@H](O)C[C@@H]2N(C)CC3)cc1OCC1CC1] | 1748 |
| 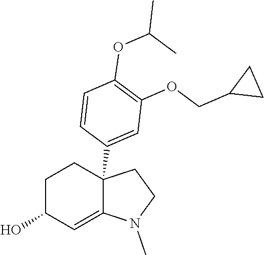 [SMILES: CC(C)Oc1ccc([C@@]23CC[C@@H](O)C=C2N(C)CC3)cc1OCC1CC1] | 1749 |
| 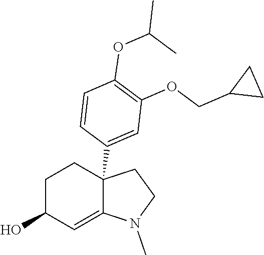 [SMILES: CC(C)Oc1ccc([C@@]23CC[C@H](O)C=C2N(C)CC3)cc1OCC1CC1] | 1750 |
| 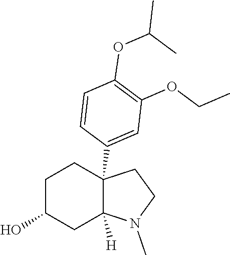 [SMILES: CCOc1cc([C@@]23CC[C@@H](O)C[C@@H]2N(C)CC3)ccc1OC(C)C] | 1751 |
| 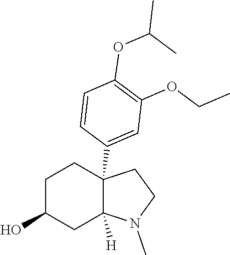 [SMILES: CCOc1cc([C@@]23CC[C@H](O)C[C@@H]2N(C)CC3)ccc1OC(C)C] | 1752 |
| 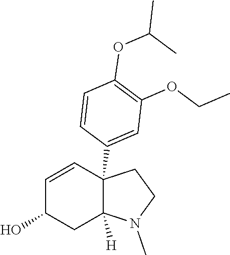 [SMILES: CCOc1cc([C@@]23C=C[C@@H](O)C[C@@H]2N(C)CC3)ccc1OC(C)C] | 1753 |
| 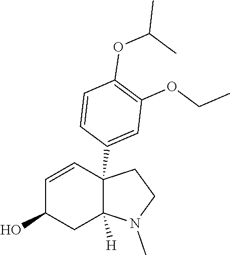 [SMILES: CCOc1cc([C@@]23C=C[C@H](O)C[C@@H]2N(C)CC3)ccc1OC(C)C] | 1754 |
| 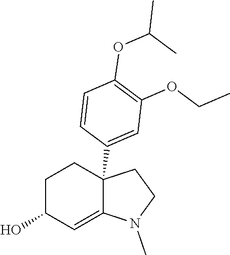 [SMILES: CCOc1cc([C@@]23CC[C@@H](O)C=C2N(C)CC3)ccc1OC(C)C] | 1755 |
| 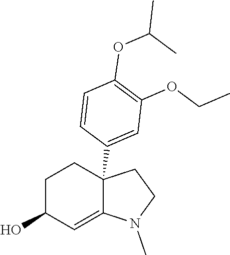 [SMILES: CCOc1cc([C@@]23CC[C@H](O)C=C2N(C)CC3)ccc1OC(C)C] | 1756 |
| 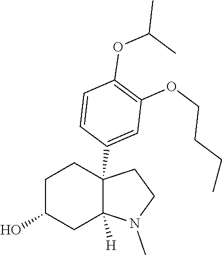 [SMILES: CCCCOc1cc([C@@]23CC[C@@H](O)C[C@@H]2N(C)CC3)ccc1OC(C)C] | 1757 |
| 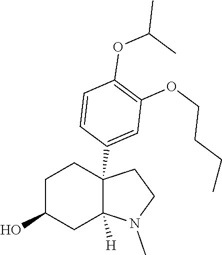 [SMILES: CCCCOc1cc([C@@]23CC[C@H](O)C[C@@H]2N(C)CC3)ccc1OC(C)C] | 1758 |
| 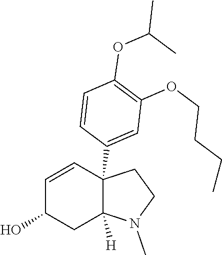 [SMILES: CCCCOc1cc([C@@]23C=C[C@@H](O)C[C@@H]2N(C)CC3)ccc1OC(C)C] | 1759 |
| 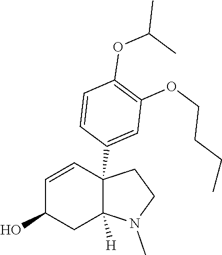 [SMILES: CCCCOc1cc([C@@]23C=C[C@H](O)C[C@@H]2N(C)CC3)ccc1OC(C)C] | 1760 |
| 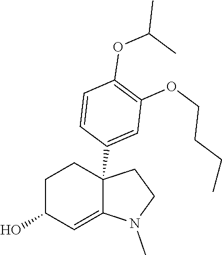 [SMILES: CCCCOc1cc([C@@]23CC[C@@H](O)C=C2N(C)CC3)ccc1OC(C)C] | 1761 |
| 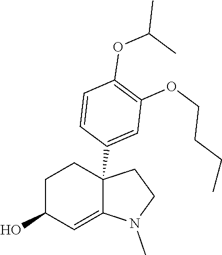 [SMILES: CCCCOc1cc([C@@]23CC[C@H](O)C=C2N(C)CC3)ccc1OC(C)C] | 1762 |
| 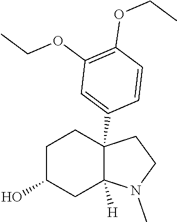 [SMILES: CCOc1ccc([C@@]23CC[C@@H](O)C[C@@H]2N(C)CC3)cc1OCC] | 1763 |
| 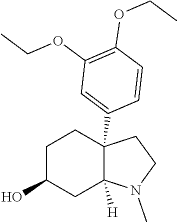 [SMILES: CCOc1ccc([C@@]23CC[C@H](O)C[C@@H]2N(C)CC3)cc1OCC] | 1764 |
| 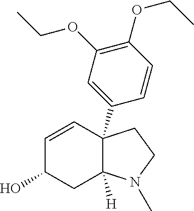 [SMILES: CCOc1ccc([C@@]23C=C[C@@H](O)C[C@@H]2N(C)CC3)cc1OCC] | 1765 |
| 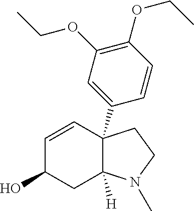 [SMILES: CCOc1ccc([C@@]23C=C[C@H](O)C[C@@H]2N(C)CC3)cc1OCC] | 1766 |
| 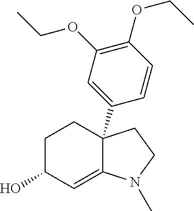 [SMILES: CCOc1ccc([C@@]23CC[C@@H](O)C=C2N(C)CC3)cc1OCC] | 1767 |
| 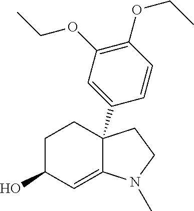 [SMILES: CCOc1ccc([C@@]23CC[C@H](O)C=C2N(C)CC3)cc1OCC] | 1768 |
| 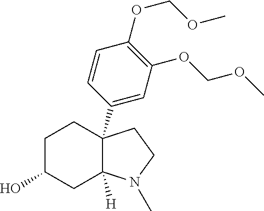 [SMILES: COCOc1ccc([C@@]23CC[C@@H](O)C[C@@H]2N(C)CC3)cc1OCOC] | 1769 |
| 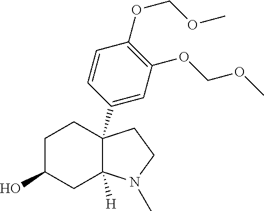 [SMILES: COCOc1ccc([C@@]23CC[C@H](O)C[C@@H]2N(C)CC3)cc1OCOC] | 1770 |
| 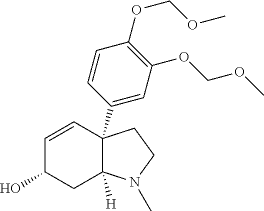 [SMILES: COCOc1ccc([C@@]23C=C[C@@H](O)C[C@@H]2N(C)CC3)cc1OCOC] | 1771 |
| 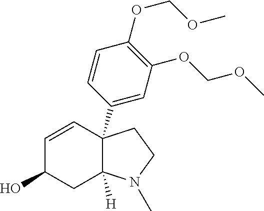 [SMILES: COCOc1ccc([C@@]23C=C[C@H](O)C[C@@H]2N(C)CC3)cc1OCOC] | 1772 |
| 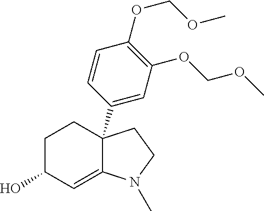 [SMILES: COCOc1ccc([C@@]23CC[C@@H](O)C=C2N(C)CC3)cc1OCOC] | 1773 |
| 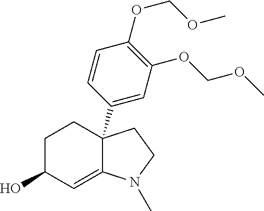 [SMILES: COCOc1ccc([C@@]23CC[C@H](O)C=C2N(C)CC3)cc1OCOC] | 1774 |
| 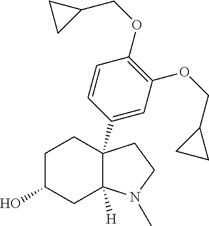 [SMILES: CN1CC[C@]2(c3ccc(OCC4CC4)c(OCC4CC4)c3)CC[C@@H](O)C[C@H]12] | 1775 |
| 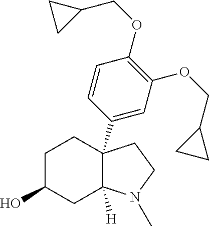 [SMILES: CN1CC[C@]2(c3ccc(OCC4CC4)c(OCC4CC4)c3)CC[C@H](O)C[C@H]12] | 1776 |
| 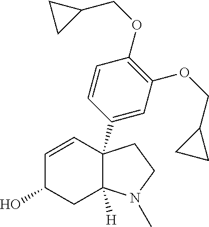 [SMILES: CN1CC[C@]2(c3ccc(OCC4CC4)c(OCC4CC4)c3)C=C[C@@H](O)C[C@H]12] | 1777 |
| 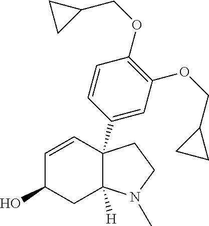 [SMILES: CN1CC[C@]2(c3ccc(OCC4CC4)c(OCC4CC4)c3)C=C[C@H](O)C[C@H]12] | 1778 |
| 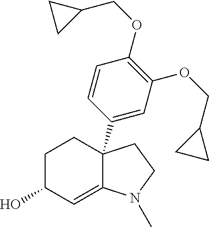 [SMILES: CN1CC[C@]2(c3ccc(OCC4CC4)c(OCC4CC4)c3)CC[C@@H](O)C=C12] | 1779 |
| 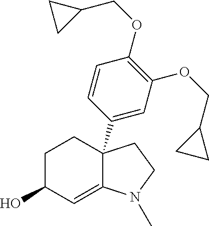 [SMILES: CN1CC[C@]2(c3ccc(OCC4CC4)c(OCC4CC4)c3)CC[C@H](O)C=C12] | 1780 |
| 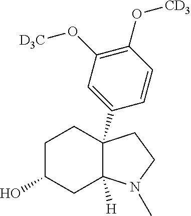 [SMILES: [2H]C([2H])([2H])Oc1ccc([C@@]23CC[C@@H](O)C[C@@H]2N(C)CC3)cc1OC([2H])([2H])[2H]] | 1781 |
| 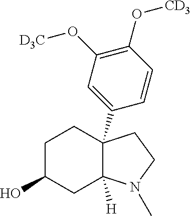 [SMILES: [2H]C([2H])([2H])Oc1ccc([C@@]23CC[C@H](O)C[C@@H]2N(C)CC3)cc1OC([2H])([2H])[2H]] | 1782 |
| 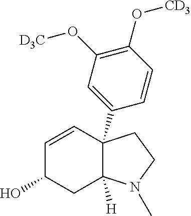 [SMILES: [2H]C([2H])([2H])Oc1ccc([C@@]23C=C[C@@H](O)C[C@@H]2N(C)CC3)cc1OC([2H])([2H])[2H]] | 1783 |
| 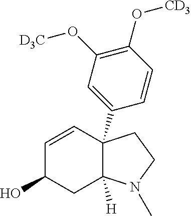 [SMILES: [2H]C([2H])([2H])Oc1ccc([C@@]23C=C[C@H](O)C[C@@H]2N(C)CC3)cc1OC([2H])([2H])[2H]] | 1784 |
| 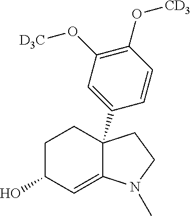 [SMILES: [2H]C([2H])([2H])Oc1ccc([C@@]23CC[C@@H](O)C=C2N(C)CC3)cc1OC([2H])([2H])[2H]] | 1785 |
| 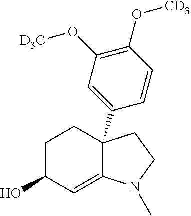 [SMILES: [2H]C([2H])([2H])Oc1ccc([C@@]23CC[C@H](O)C=C2N(C)CC3)cc1OC([2H])([2H])[2H]] | 1786 |
| 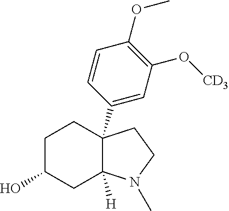 [SMILES: [2H]C([2H])([2H])Oc1cc([C@@]23CC[C@@H](O)C[C@@H]2N(C)CC3)ccc1OC] | 1787 |
| 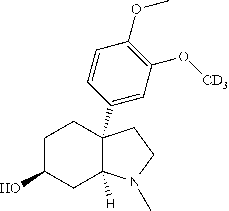 [SMILES: [2H]C([2H])([2H])Oc1cc([C@@]23CC[C@H](O)C[C@@H]2N(C)CC3)ccc1OC] | 1788 |
| 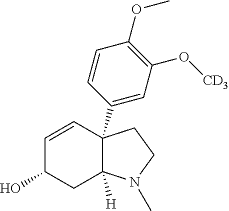 [SMILES: [2H]C([2H])([2H])Oc1cc([C@@]23C=C[C@@H](O)C[C@@H]2N(C)CC3)ccc1OC] | 1789 |
| 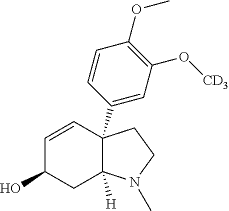 [SMILES: [2H]C([2H])([2H])Oc1cc([C@@]23C=C[C@H](O)C[C@@H]2N(C)CC3)ccc1OC] | 1790 |
| 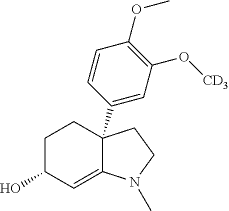 [SMILES: [2H]C([2H])([2H])Oc1cc([C@@]23CC[C@@H](O)C=C2N(C)CC3)ccc1OC] | 1791 |
| 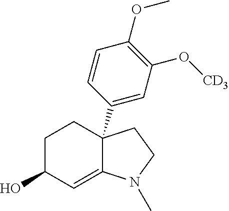 [SMILES: [2H]C([2H])([2H])Oc1cc([C@@]23CC[C@H](O)C=C2N(C)CC3)ccc1OC] | 1792 |
| 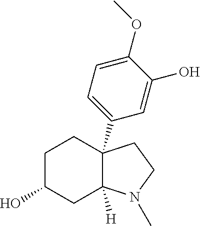 [SMILES: COc1ccc([C@@]23CC[C@@H](O)C[C@@H]2N(C)CC3)cc1O] | 1793 |
| 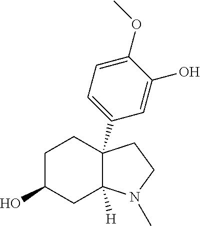 [SMILES: COc1ccc([C@@]23CC[C@H](O)C[C@@H]2N(C)CC3)cc1O] | 1794 |
| 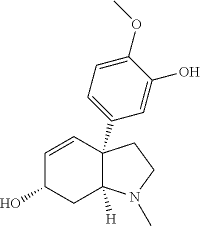 [SMILES: COc1ccc([C@@]23C=C[C@@H](O)C[C@@H]2N(C)CC3)cc1O] | 1795 |
| 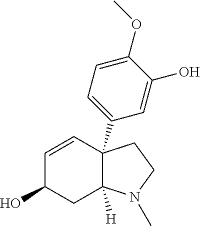 [SMILES: COc1ccc([C@@]23C=C[C@H](O)C[C@@H]2N(C)CC3)cc1O] | 1796 |
| 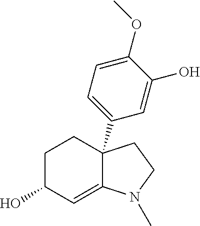 [SMILES: COc1ccc([C@@]23CC[C@@H](O)C=C2N(C)CC3)cc1O] | 1797 |
| 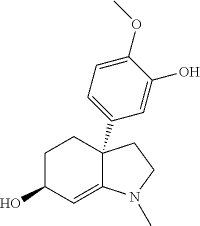 [SMILES: COc1ccc([C@@]23CC[C@H](O)C=C2N(C)CC3)cc1O] | 1798 |
| 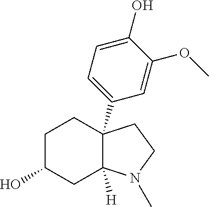 [SMILES: COc1cc([C@@]23CC[C@@H](O)C[C@@H]2N(C)CC3)ccc1O] | 1799 |
| 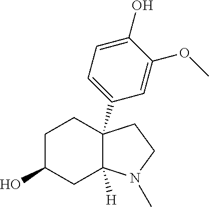 [SMILES: COc1cc([C@@]23CC[C@H](O)C[C@@H]2N(C)CC3)ccc1O] | 2700 |
| 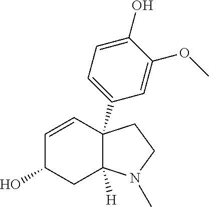 [SMILES: COc1cc([C@@]23C=C[C@@H](O)C[C@@H]2N(C)CC3)ccc1O] | 2701 |
| 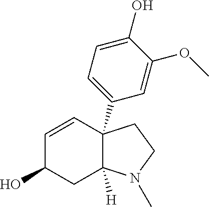 [SMILES: COc1cc([C@@]23C=C[C@H](O)C[C@@H]2N(C)CC3)ccc1O] | 2702 |
| 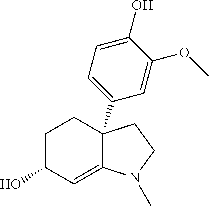 [SMILES: COc1cc([C@@]23CC[C@@H](O)C=C2N(C)CC3)ccc1O] | 2703 |
| 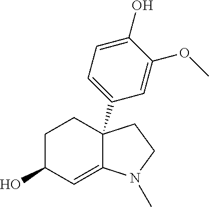 [SMILES: COc1cc([C@@]23CC[C@H](O)C=C2N(C)CC3)ccc1O] | 2704 |
| 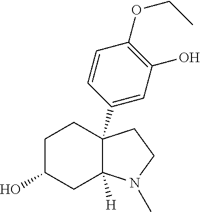 [SMILES: CCOc1ccc([C@@]23CC[C@@H](O)C[C@@H]2N(C)CC3)cc1O] | 2705 |
| 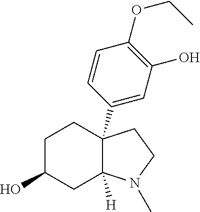 [SMILES: CCOc1ccc([C@@]23CC[C@H](O)C[C@@H]2N(C)CC3)cc1O] | 2706 |
| 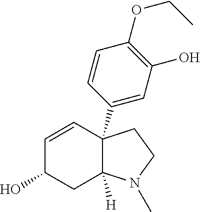 [SMILES: CCOc1ccc([C@@]23C=C[C@@H](O)C[C@@H]2N(C)CC3)cc1O] | 2707 |
| 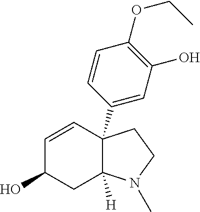 [SMILES: CCOc1ccc([C@@]23C=C[C@H](O)C[C@@H]2N(C)CC3)cc1O] | 2708 |
| 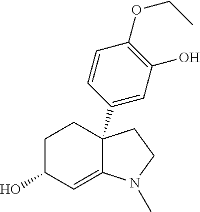 [SMILES: CCOc1ccc([C@@]23CC[C@@H](O)C=C2N(C)CC3)cc1O] | 2709 |
| 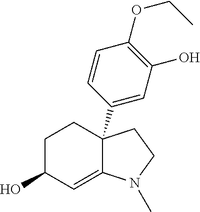 [SMILES: CCOc1ccc([C@@]23CC[C@H](O)C=C2N(C)CC3)cc1O] | 2710 |
| 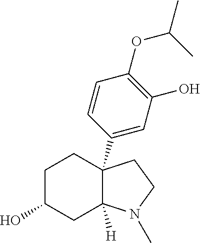 [SMILES: CC(C)Oc1ccc([C@@]23CC[C@@H](O)C[C@@H]2N(C)CC3)cc1O] | 2711 |
| 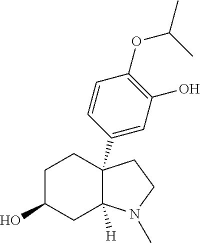 [SMILES: CC(C)Oc1ccc([C@@]23CC[C@H](O)C[C@@H]2N(C)CC3)cc1O] | 2712 |
| 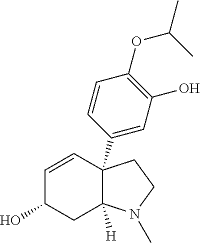 [SMILES: CC(C)Oc1ccc([C@@]23C=C[C@@H](O)C[C@@H]2N(C)CC3)cc1O] | 2713 |
| 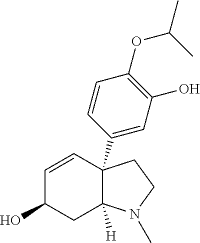 [SMILES: CC(C)Oc1ccc([C@@]23C=C[C@H](O)C[C@@H]2N(C)CC3)cc1O] | 2714 |
| 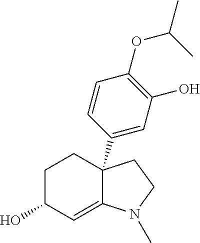 [SMILES: CC(C)Oc1ccc([C@@]23CC[C@@H](O)C=C2N(C)CC3)cc1O] | 2715 |
| 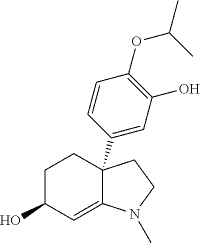 [SMILES: CC(C)Oc1ccc([C@@]23CC[C@H](O)C=C2N(C)CC3)cc1O] | 2716 |
| 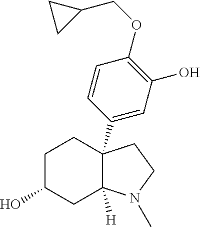 [SMILES: CN1CC[C@]2(c3ccc(OCC4CC4)c(O)c3)CC[C@@H](O)C[C@H]12] | 2717 |
| 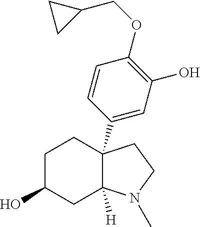 [SMILES: CN1CC[C@]2(c3ccc(OCC4CC4)c(O)c3)CC[C@H](O)C[C@H]12] | 2718 |
| 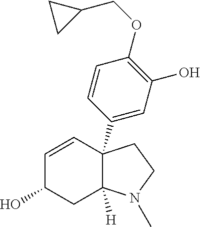 [SMILES: CN1CC[C@]2(c3ccc(OCC4CC4)c(O)c3)C=C[C@@H](O)C[C@H]12] | 2719 |
| 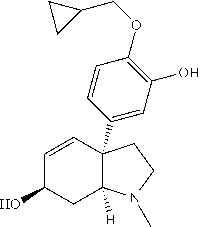 [SMILES: CN1CC[C@]2(c3ccc(OCC4CC4)c(O)c3)C=C[C@H](O)C[C@H]12] | 2720 |
| 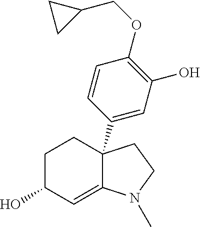 [SMILES: CN1CC[C@]2(c3ccc(OCC4CC4)c(O)c3)CC[C@@H](O)C=C12] | 2721 |
| 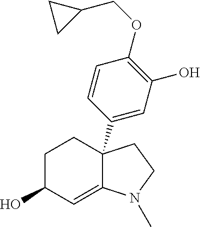 [SMILES: CN1CC[C@]2(c3ccc(OCC4CC4)c(O)c3)CC[C@H](O)C=C12] | 2722 |
| 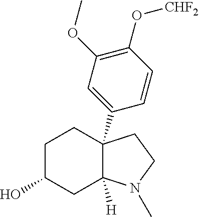 [SMILES: COc1cc([C@@]23CC[C@@H](O)C[C@@H]2N(C)CC3)ccc1OC(F)F] | 2723 |
| 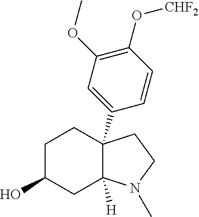 [SMILES: COc1cc([C@@]23CC[C@H](O)C[C@@H]2N(C)CC3)ccc1OC(F)F] | 2724 |
| 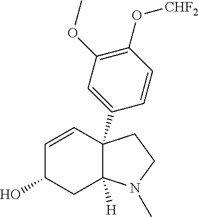 [SMILES: COc1cc([C@@]23C=C[C@@H](O)C[C@@H]2N(C)CC3)ccc1OC(F)F] | 2725 |
| 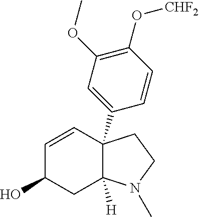 [SMILES: COc1cc([C@@]23C=C[C@H](O)C[C@@H]2N(C)CC3)ccc1OC(F)F] | 2726 |
| 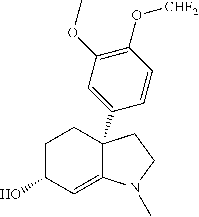 [SMILES: COc1cc([C@@]23CC[C@@H](O)C=C2N(C)CC3)ccc1OC(F)F] | 2727 |
| 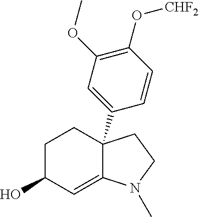 [SMILES: COc1cc([C@@]23CC[C@H](O)C=C2N(C)CC3)ccc1OC(F)F] | 2728 |
| 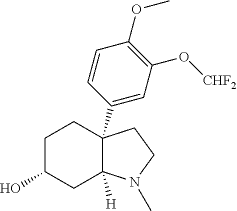 [SMILES: COc1ccc([C@@]23CC[C@@H](O)C[C@@H]2N(C)CC3)cc1OC(F)F] | 2729 |
| 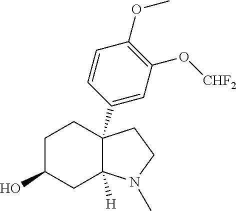 [SMILES: COc1ccc([C@@]23CC[C@H](O)C[C@@H]2N(C)CC3)cc1OC(F)F] | 2730 |
| 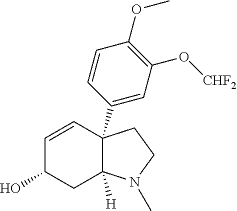 [SMILES: COc1ccc([C@@]23C=C[C@@H](O)C[C@@H]2N(C)CC3)cc1OC(F)F] | 2731 |
| 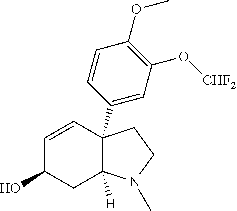 [SMILES: COc1ccc([C@@]23C=C[C@H](O)C[C@@H]2N(C)CC3)cc1OC(F)F] | 2732 |
| 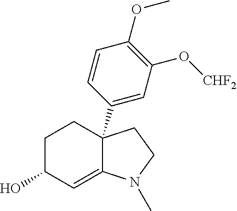 [SMILES: COc1ccc([C@@]23CC[C@@H](O)C=C2N(C)CC3)cc1OC(F)F] | 2733 |
| 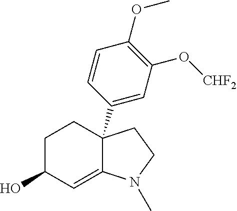 [SMILES: COc1ccc([C@@]23CC[C@H](O)C=C2N(C)CC3)cc1OC(F)F] | 2734 |
| 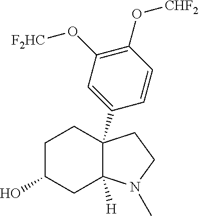 [SMILES: CN1CC[C@]2(c3ccc(OC(F)F)c(OC(F)F)c3)CC[C@@H](O)C[C@H]12] | 2735 |
| 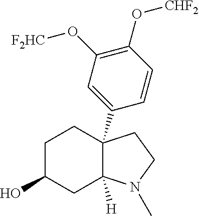 [SMILES: CN1CC[C@]2(c3ccc(OC(F)F)c(OC(F)F)c3)CC[C@H](O)C[C@H]12] | 2736 |
| 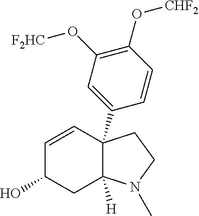 [SMILES: CN1CC[C@]2(c3ccc(OC(F)F)c(OC(F)F)c3)C=C[C@@H](O)C[C@H]12] | 2737 |
| 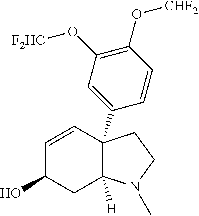 [SMILES: CN1CC[C@]2(c3ccc(OC(F)F)c(OC(F)F)c3)C=C[C@H](O)C[C@H]12] | 2738 |
| 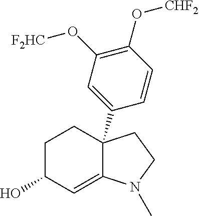 [SMILES: CN1CC[C@]2(c3ccc(OC(F)F)c(OC(F)F)c3)CC[C@@H](O)C=C12] | 2739 |
| 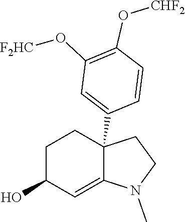 [SMILES: CN1CC[C@]2(c3ccc(OC(F)F)c(OC(F)F)c3)CC[C@H](O)C=C12] | 2740 |
| 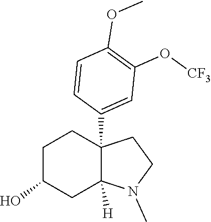 [SMILES: COc1ccc([C@@]23CC[C@@H](O)C[C@@H]2N(C)CC3)cc1OC(F)(F)F] | 2741 |
| 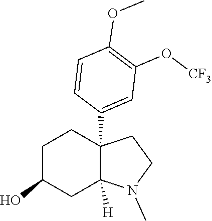 [SMILES: COc1ccc([C@@]23CC[C@H](O)C[C@@H]2N(C)CC3)cc1OC(F)(F)F] | 2742 |
| 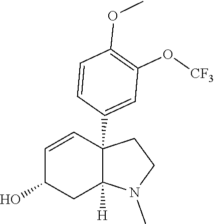 [SMILES: COc1ccc([C@@]23C=C[C@@H](O)C[C@@H]2N(C)CC3)cc1OC(F)(F)F] | 2743 |
| 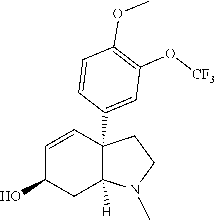 [SMILES: COc1ccc([C@@]23C=C[C@H](O)C[C@@H]2N(C)CC3)cc1OC(F)(F)F] | 2744 |
| 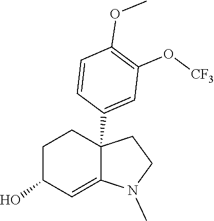 [SMILES: COc1ccc([C@@]23CC[C@@H](O)C=C2N(C)CC3)cc1OC(F)(F)F] | 2745 |
| 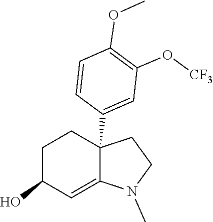 [SMILES: COc1ccc([C@@]23CC[C@H](O)C=C2N(C)CC3)cc1OC(F)(F)F] | 2746 |
| 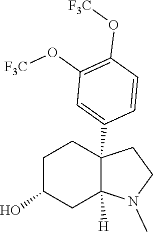 [SMILES: CN1CC[C@]2(c3ccc(OC(F)(F)F)c(OC(F)(F)F)c3)CC[C@@H](O)C[C@H]12] | 2747 |
| 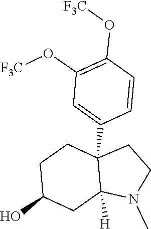 [SMILES: CN1CC[C@]2(c3ccc(OC(F)(F)F)c(OC(F)(F)F)c3)CC[C@H](O)C[C@H]12] | 2748 |
| 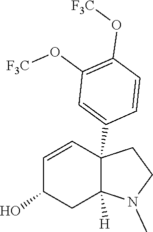 [SMILES: CN1CC[C@]2(c3ccc(OC(F)(F)F)c(OC(F)(F)F)c3)C=C[C@@H](O)C[C@H]12] | 2749 |
| 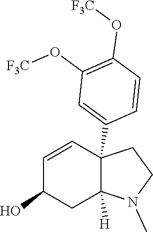 [SMILES: CN1CC[C@]2(c3ccc(OC(F)(F)F)c(OC(F)(F)F)c3)C=C[C@H](O)C[C@H]12] | 2750 |
| 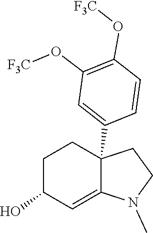 [SMILES: CN1CC[C@]2(c3ccc(OC(F)(F)F)c(OC(F)(F)F)c3)CC[C@@H](O)C=C12] | 2751 |
| 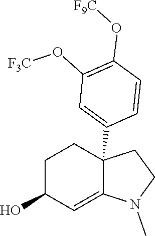 | 2752 |
| 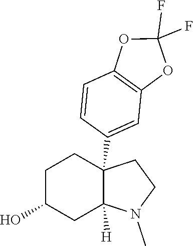 [SMILES: CN1CC[C@]2(c3ccc4c(c3)OC(F)(F)O4)CC[C@@H](O)C[C@H]12] | 2753 |
| 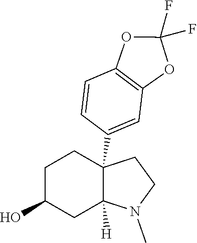 [SMILES: CN1CC[C@]2(c3ccc4c(c3)OC(F)(F)O4)CC[C@H](O)C[C@H]12] | 2754 |
| 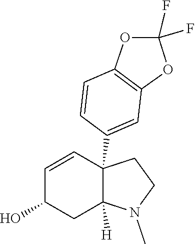 [SMILES: CN1CC[C@]2(c3ccc4c(c3)OC(F)(F)O4)C=C[C@@H](O)C[C@H]12] | 2755 |
| 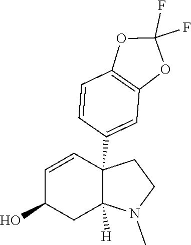 [SMILES: CN1CC[C@]2(c3ccc4c(c3)OC(F)(F)O4)C=C[C@H](O)C[C@H]12] | 2756 |
| 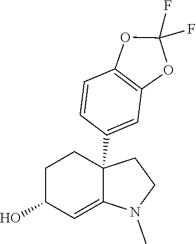 [SMILES: CN1CC[C@]2(c3ccc4c(c3)OC(F)(F)O4)CC[C@@H](O)C=C12] | 2757 |
| 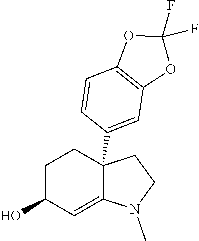 [SMILES: CN1CC[C@]2(c3ccc4c(c3)OC(F)(F)O4)CC[C@H](O)C=C12] | 2758 |
| 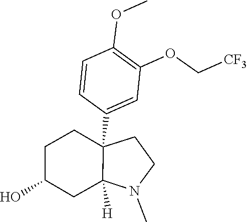 [SMILES: COc1ccc([C@@]23CC[C@@H](O)C[C@@H]2N(C)CC3)cc1OCC(F)(F)F] | 2759 |
| 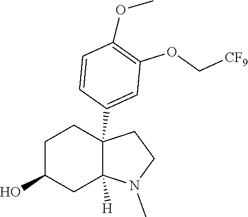 | 2760 |
| 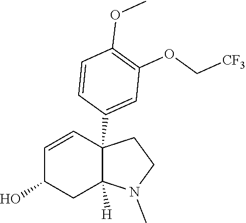 [SMILES: COc1ccc([C@@]23C=C[C@@H](O)C[C@@H]2N(C)CC3)cc1OCC(F)(F)F] | 2761 |
| 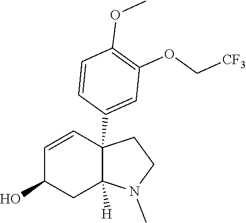 [SMILES: COc1ccc([C@@]23C=C[C@H](O)C[C@@H]2N(C)CC3)cc1OCC(F)(F)F] | 2762 |
| 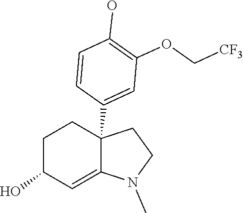 | 2763 |
| 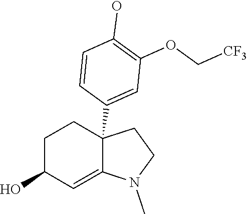 | 2764 |
| 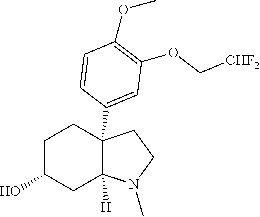 [SMILES: COc1ccc([C@@]23CC[C@@H](O)C[C@@H]2N(C)CC3)cc1OCC(F)F] | 2765 |
| 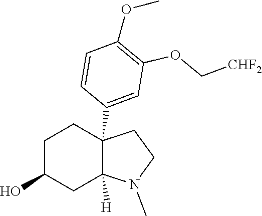 [SMILES: COc1ccc([C@@]23CC[C@H](O)C[C@@H]2N(C)CC3)cc1OCC(F)F] | 2766 |
| 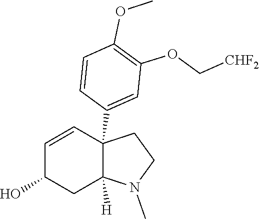 [SMILES: COc1ccc([C@@]23C=C[C@@H](O)C[C@@H]2N(C)CC3)cc1OCC(F)F] | 2767 |
| 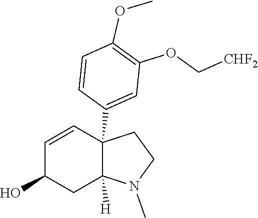 [SMILES: COc1ccc([C@@]23C=C[C@H](O)C[C@@H]2N(C)CC3)cc1OCC(F)F] | 2768 |
| 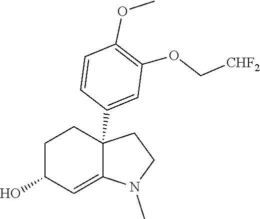 [SMILES: COc1ccc([C@@]23CC[C@@H](O)C=C2N(C)CC3)cc1OCC(F)F] | 2769 |
| 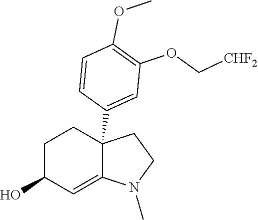 [SMILES: COc1ccc([C@@]23CC[C@H](O)C=C2N(C)CC3)cc1OCC(F)F] | 2770 |
| 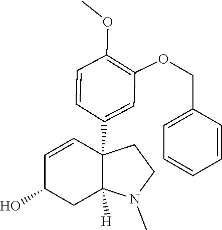 [SMILES: COc1ccc([C@@]23C=C[C@@H](O)C[C@@H]2N(C)CC3)cc1OCc1ccccc1] | 2771 |
| 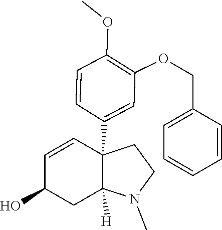 [SMILES: COc1ccc([C@@]23C=C[C@H](O)C[C@@H]2N(C)CC3)cc1OCc1ccccc1] | 2772 |
| 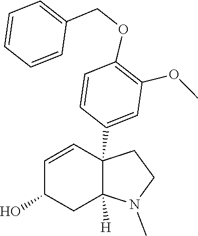 [SMILES: COc1cc([C@@]23C=C[C@@H](O)C[C@@H]2N(C)CC3)ccc1OCc1ccccc1] | 2773 |
| 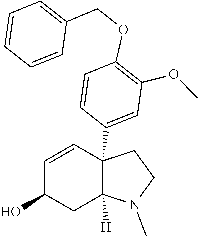 [SMILES: COc1cc([C@@]23C=C[C@H](O)C[C@@H]2N(C)CC3)ccc1OCc1ccccc1] | 2774 |

or a pharmaceutically acceptable salt thereof.

In certain embodiments, the compound is of Formula (IA-8), (IB-8) or (IC-8):

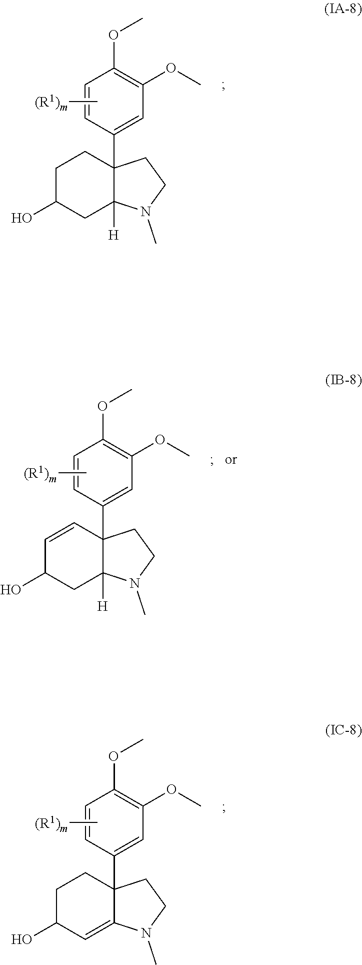

or a pharmaceutically acceptable salt thereof.

In certain embodiments, the compound is of Formula (IA-8) or a pharmaceutically acceptable salt thereof.

In certain embodiments, the compound is of Formula (IB-8) or a pharmaceutically acceptable salt thereof.

In certain embodiments, the compound is of Formula (IC-8) or a pharmaceutically acceptable salt thereof.

In certain embodiments, the compound is of Formula (IA-8a), (IA-8b), (IB-8a), (IB-8b), (IC-8a) or (IC-8b):

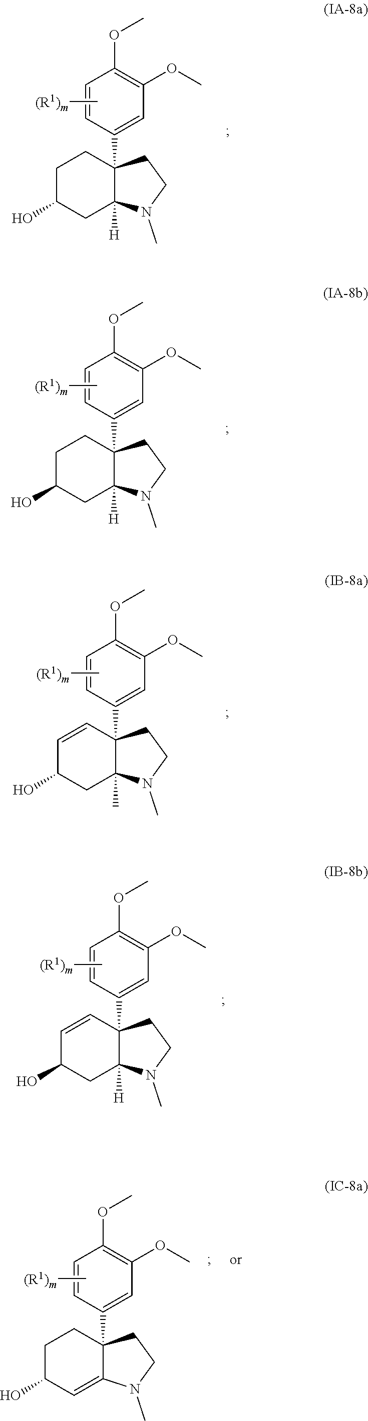

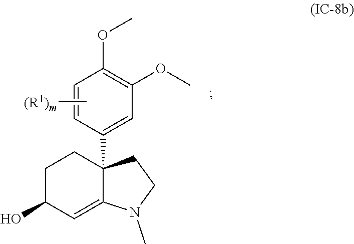

or a pharmaceutically acceptable salt thereof.

In certain embodiments, the compounds of Formula (IA-8a), (IA-8b), (IB-8a), (IB-8b), (IC-8a) and (IC-8b) have the absolute stereochemistry shown.

In certain embodiments, the compound is of Formula (IA-8a) or a pharmaceutically acceptable salt thereof.

In certain embodiments, the compound is of Formula (IA-8b) or a pharmaceutically acceptable salt thereof.

In certain embodiments, the compound is of Formula (IB-8a) or a pharmaceutically acceptable salt thereof.

In certain embodiments, the compound is of Formula (IB-8b) or a pharmaceutically acceptable salt thereof.

In certain embodiments, the compound is of Formula (IC-8a) or a pharmaceutically acceptable salt thereof.

In certain embodiments, the compound is of Formula (IC-8b) or a pharmaceutically acceptable salt thereof.

In certain embodiments, m is 1, 2, or 3. In certain embodiments, m is 1.

In certain embodiments, R¹ is halo, alkenyl, cycloalkyl, cyano, or —C(O)NR^{a}R^{b}. For example, in certain embodiments, R¹ is fluoro, chloro, bromo, iodo, vinyl, cyclopropyl, cyano, or —C(O)NH₂.

In certain embodiments, the compound is selected from the group consisting of:

TABLE 5
Compounds of Formula IA-8a, IA-8b, IB-8a, IB-8b, IC-8a and IC-8b

| | |
|---|---|
| 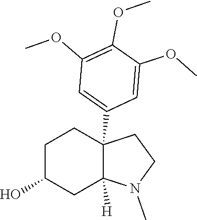 [SMILES: COc1cc([C@@]23CC[C@@H](O)C[C@@H]2N(C)CC3)cc(OC)c1OC] | 0801 |
| 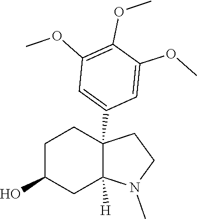 [SMILES: COc1cc([C@@]23CC[C@H](O)C[C@@H]2N(C)CC3)cc(OC)c1OC] | 0802 |
| 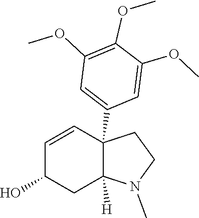 [SMILES: COc1cc([C@@]23C=C[C@@H](O)C[C@@H]2N(C)CC3)cc(OC)c1OC] | 0803 |
| 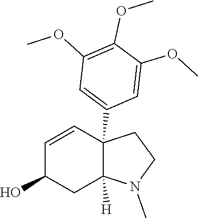 [SMILES: COc1cc([C@@]23C=C[C@H](O)C[C@@H]2N(C)CC3)cc(OC)c1OC] | 0804 |
| 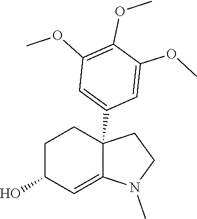 [SMILES: COc1cc([C@@]23CC[C@@H](O)C=C2N(C)CC3)cc(OC)c1OC] | 0805 |
| 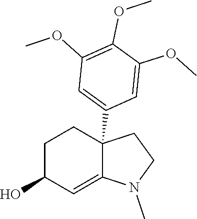 [SMILES: COc1cc([C@@]23CC[C@H](O)C=C2N(C)CC3)cc(OC)c1OC] | 0806 |
| 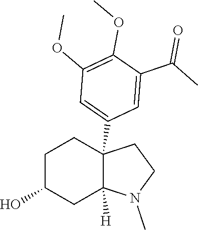 [SMILES: COc1cc([C@@]23CC[C@@H](O)C[C@@H]2N(C)CC3)cc(C(C)=O)c1OC] | 0807 |
| 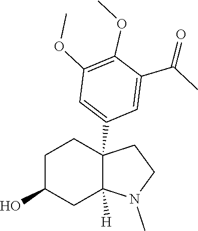 [SMILES: COc1cc([C@@]23CC[C@H](O)C[C@@H]2N(C)CC3)cc(C(C)=O)c1OC] | 0808 |
| 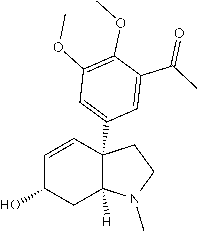 [SMILES: COc1cc([C@@]23C=C[C@@H](O)C[C@@H]2N(C)CC3)cc(C(C)=O)c1OC] | 0809 |
| 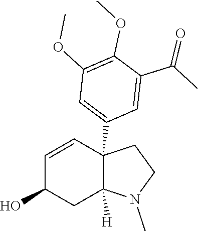 [SMILES: COc1cc([C@@]23C=C[C@H](O)C[C@@H]2N(C)CC3)cc(C(C)=O)c1OC] | 0810 |
| 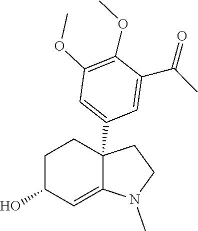 [SMILES: COc1cc([C@@]23CC[C@@H](O)C=C2N(C)CC3)cc(C(C)=O)c1OC] | 0811 |
| 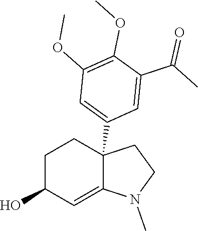 [SMILES: COc1cc([C@@]23CC[C@H](O)C=C2N(C)CC3)cc(C(C)=O)c1OC] | 0812 |
| 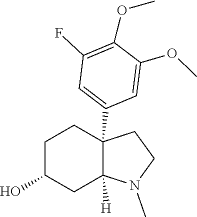 [SMILES: COc1cc([C@@]23CC[C@@H](O)C[C@@H]2N(C)CC3)cc(F)c1OC] | 0813 |
| 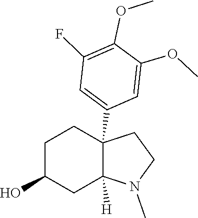 [SMILES: COc1cc([C@@]23CC[C@H](O)C[C@@H]2N(C)CC3)cc(F)c1OC] | 0814 |
| 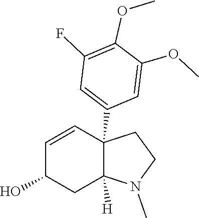 [SMILES: COc1cc([C@@]23C=C[C@@H](O)C[C@@H]2N(C)CC3)cc(F)c1OC] | 0815 |
| 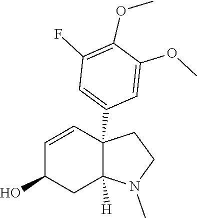 [SMILES: COc1cc([C@@]23C=C[C@H](O)C[C@@H]2N(C)CC3)cc(F)c1OC] | 0816 |
| 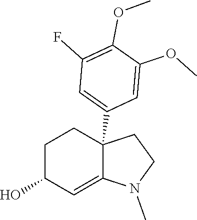 [SMILES: COc1cc([C@@]23CC[C@@H](O)C=C2N(C)CC3)cc(F)c1OC] | 0817 |
| 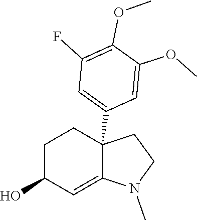 [SMILES: COc1cc([C@@]23CC[C@H](O)C=C2N(C)CC3)cc(F)c1OC] | 0818 |
| 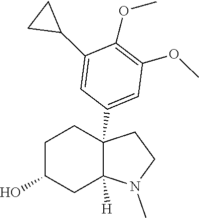 [SMILES: COc1cc([C@@]23CC[C@@H](O)C[C@@H]2N(C)CC3)cc(C2CC2)c1OC] | 0819 |
| 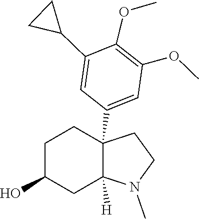 [SMILES: COc1cc([C@@]23CC[C@H](O)C[C@@H]2N(C)CC3)cc(C2CC2)c1OC] | 0820 |
| 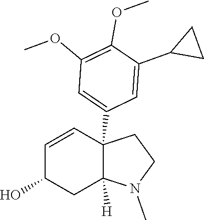 [SMILES: COc1cc([C@@]23C=C[C@@H](O)C[C@@H]2N(C)CC3)cc(C2CC2)c1OC] | 0821 |
| 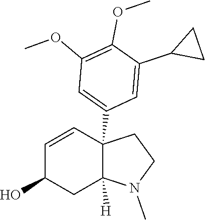 [SMILES: COc1cc([C@@]23C=C[C@H](O)C[C@@H]2N(C)CC3)cc(C2CC2)c1OC] | 0822 |
| 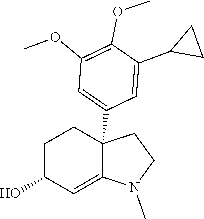 [SMILES: COc1cc([C@@]23CC[C@@H](O)C=C2N(C)CC3)cc(C2CC2)c1OC] | 0823 |
| 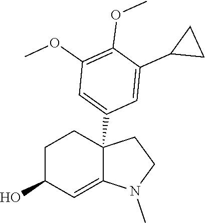 [SMILES: COc1cc([C@@]23CC[C@H](O)C=C2N(C)CC3)cc(C2CC2)c1OC] | 0824 |
| 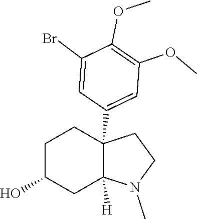 [SMILES: COc1cc([C@@]23CC[C@@H](O)C[C@@H]2N(C)CC3)cc(Br)c1OC] | 0825 |
| 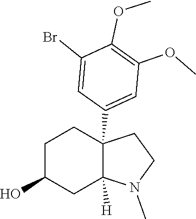 [SMILES: COc1cc([C@@]23CC[C@H](O)C[C@@H]2N(C)CC3)cc(Br)c1OC] | 0826 |
| 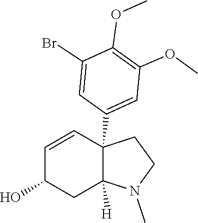 [SMILES: COc1cc([C@@]23C=C[C@@H](O)C[C@@H]2N(C)CC3)cc(Br)c1OC] | 0827 |
| 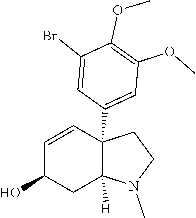 [SMILES: COc1cc([C@@]23C=C[C@H](O)C[C@@H]2N(C)CC3)cc(Br)c1OC] | 0828 |
| 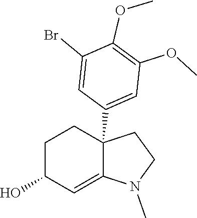 [SMILES: COc1cc([C@@]23CC[C@@H](O)C=C2N(C)CC3)cc(Br)c1OC] | 0829 |
| 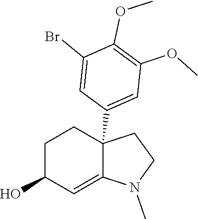 [SMILES: COc1cc([C@@]23CC[C@H](O)C=C2N(C)CC3)cc(Br)c1OC] | 0830 |
| 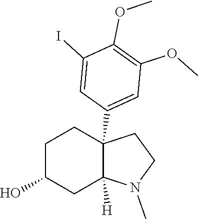 [SMILES: COc1cc([C@@]23CC[C@@H](O)C[C@@H]2N(C)CC3)cc(I)c1OC] | 0831 |
| 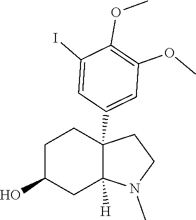 [SMILES: COc1cc([C@@]23CC[C@H](O)C[C@@H]2N(C)CC3)cc(I)c1OC] | 0832 |
| 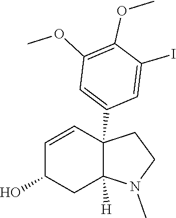 [SMILES: COc1cc([C@@]23C=C[C@@H](O)C[C@@H]2N(C)CC3)cc(I)c1OC] | 0833 |
| 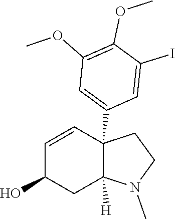 [SMILES: COc1cc([C@@]23C=C[C@H](O)C[C@@H]2N(C)CC3)cc(I)c1OC] | 0834 |
| 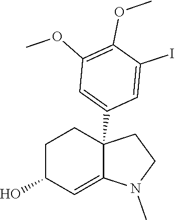 [SMILES: COc1cc([C@@]23CC[C@@H](O)C=C2N(C)CC3)cc(I)c1OC] | 0835 |
| 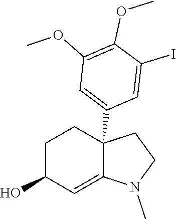 [SMILES: COc1cc([C@@]23CC[C@H](O)C=C2N(C)CC3)cc(I)c1OC] | 0836 |
| 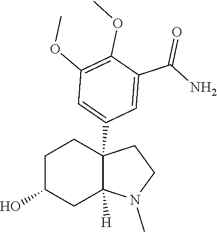 [SMILES: COc1cc([C@@]23CC[C@@H](O)C[C@@H]2N(C)CC3)cc(C(N)=O)c1OC] | 0837 |
| 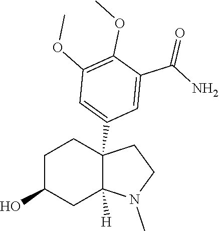 [SMILES: COc1cc([C@@]23CC[C@H](O)C[C@@H]2N(C)CC3)cc(C(N)=O)c1OC] | 0838 |
| 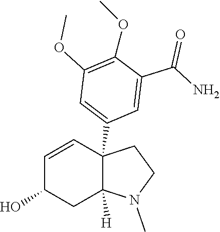 [SMILES: COc1cc([C@@]23C=C[C@@H](O)C[C@@H]2N(C)CC3)cc(C(N)=O)c1OC] | 0839 |
| 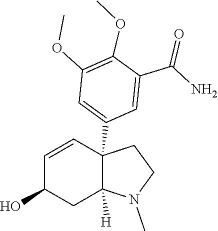 [SMILES: COc1cc([C@@]23C=C[C@H](O)C[C@@H]2N(C)CC3)cc(C(N)=O)c1OC] | 0840 |
| 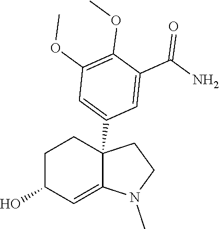 [SMILES: COc1cc([C@@]23CC[C@@H](O)C=C2N(C)CC3)cc(C(N)=O)c1OC] | 0841 |
| 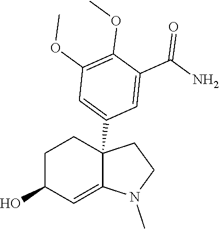 [SMILES: COc1cc([C@@]23CC[C@H](O)C=C2N(C)CC3)cc(C(N)=O)c1OC] | 0842 |
| 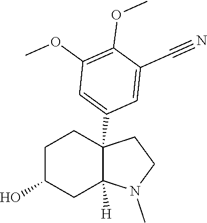 [SMILES: COc1cc([C@@]23CC[C@@H](O)C[C@@H]2N(C)CC3)cc(C#N)c1OC] | 0843 |
| 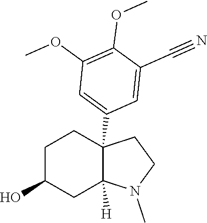 [SMILES: COc1cc([C@@]23CC[C@H](O)C[C@@H]2N(C)CC3)cc(C#N)c1OC] | 0844 |
| 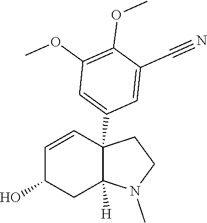 [SMILES: COc1cc([C@@]23C=C[C@@H](O)C[C@@H]2N(C)CC3)cc(C#N)c1OC] | 0845 |
| 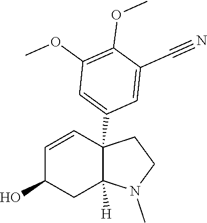 [SMILES: COc1cc([C@@]23C=C[C@H](O)C[C@@H]2N(C)CC3)cc(C#N)c1OC] | 0846 |
| 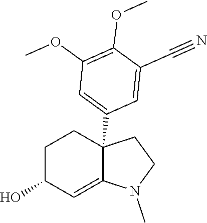 [SMILES: COc1cc([C@@]23CC[C@@H](O)C=C2N(C)CC3)cc(C#N)c1OC] | 0847 |
| 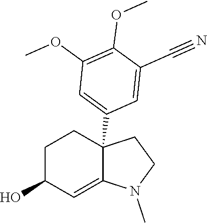 [SMILES: COc1cc([C@@]23CC[C@H](O)C=C2N(C)CC3)cc(C#N)c1OC] | 0848 |
| 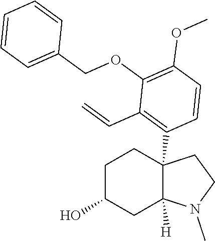 [SMILES: C=Cc1c([C@@]23CC[C@@H](O)C[C@@H]2N(C)CC3)ccc(OC)c1OCc1ccccc1] | 0849 |
| 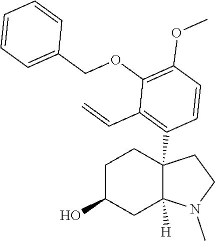 [SMILES: C=Cc1c([C@@]23CC[C@H](O)C[C@@H]2N(C)CC3)ccc(OC)c1OCc1ccccc1] | 0850 |
| 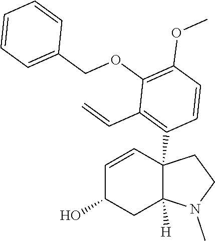 [SMILES: C=Cc1c([C@@]23C=C[C@@H](O)C[C@@H]2N(C)CC3)ccc(OC)c1OCc1ccccc1] | 0851 |
| 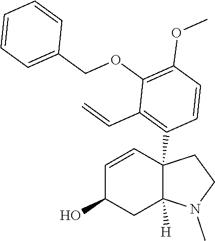 [SMILES: C=Cc1c([C@@]23C=C[C@H](O)C[C@@H]2N(C)CC3)ccc(OC)c1OCc1ccccc1] | 0852 |
| 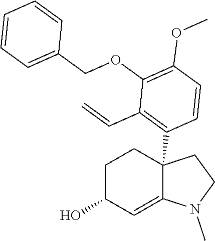 [SMILES: C=Cc1c([C@@]23CC[C@@H](O)C=C2N(C)CC3)ccc(OC)c1OCc1ccccc1] | 0853 |
| 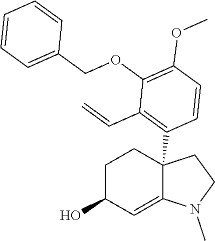 [SMILES: C=Cc1c([C@@]23CC[C@H](O)C=C2N(C)CC3)ccc(OC)c1OCc1ccccc1] | 0854 |

or a pharmaceutically acceptable salt thereof.

In certain embodiments, the compound is of Formula (IA-9), (IB-9) or (IC-9):

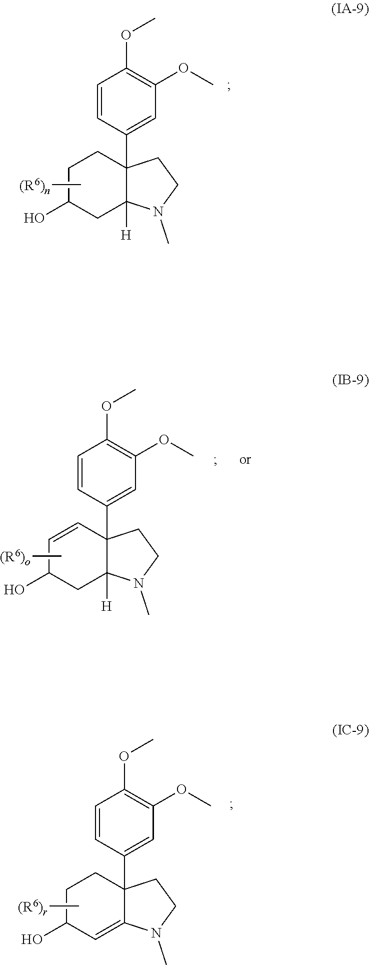

or a pharmaceutically acceptable salt thereof.

In certain embodiments, the compound is of Formula (IA-9) or a pharmaceutically acceptable salt thereof.

In certain embodiments, the compound is of Formula (IB-9) or a pharmaceutically acceptable salt thereof.

In certain embodiments, the compound is of Formula (IC-9) or a pharmaceutically acceptable salt thereof.

In certain embodiments, the compound is of Formula (IA-9a), (IA-9b), (IB-9a), (IB-9b), (IC-9a) or (IC-9b):

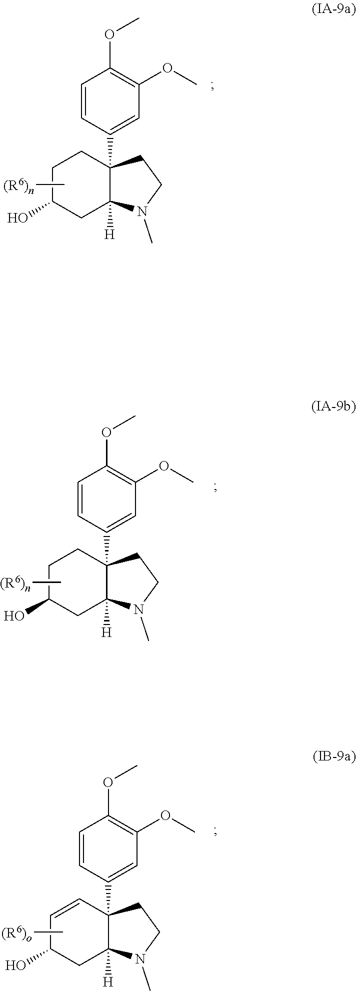

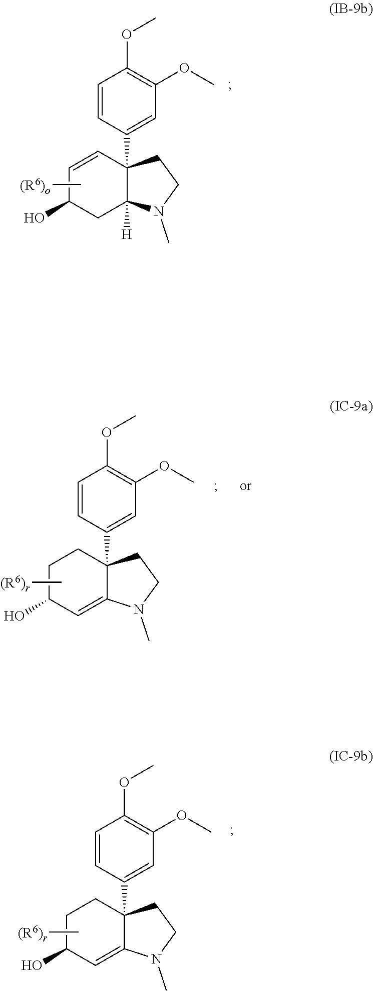

or a pharmaceutically acceptable salt thereof.

In certain embodiments, the compounds of Formula (IA-9a), (IA-9b), (IB-9a), (IB-9b), (IC-9a), and (IC-9b) have the absolute stereochemistry shown.

In certain embodiments, the compound is of Formula (IA-9a) or a pharmaceutically acceptable salt thereof.

In certain embodiments, the compound is of Formula (IA-9b) or a pharmaceutically acceptable salt thereof.

In certain embodiments, the compound is of Formula (IB-9a) or a pharmaceutically acceptable salt thereof.

In certain embodiments, the compound is of Formula (IB-9b) or a pharmaceutically acceptable salt thereof.

In certain embodiments, the compound is of Formula (IC-9a) or a pharmaceutically acceptable salt thereof.

In certain embodiments, the compound is of Formula (IC-9b) or a pharmaceutically acceptable salt thereof.

In certain embodiments, n is 1, 2, 3, 4, 5, or 6. For example, n may be 1, 2, or 3.

In certain embodiments, o is 1, 2, 3, or 4. For example, o may be 1, 2, or 3.

In certain embodiments, r is 1, 2, 3, 4, or 5. For example, r may be 1, 2 or 3.

In certain embodiments, R⁶ is deuterium, C₁-C₆ alkyl, —Si(C₁-C₆ alkyl)₃, phenyl, or —C(O)C₁-C₆ alkyl, each of which can be optionally substituted. For example, in certain embodiments, R⁶ is CF₃.

In certain embodiments, two R⁶s on a single carbon atom together with the carbon atom to which they are attached combine to form C₃-C₆ cycloalkyl. For example, two R⁶s on a single carbon atom together with the carbon atom to which they are attached combine to form cyclopropyl, cyclobutyl, or cyclohexyl, each of which can be optionally substituted.

In certain embodiments, two R⁶s on different carbon atoms together with the carbon atoms to which they are attached combine to form a C₃-C₈ cycloalkyl or 3-7 membered heterocyclyl. For example two R⁶s on different carbon atoms together with the carbon atoms to which they are attached combine to form cyclopropyl, cyclobutyl, or cyclohexyl. Alternatively for example, two R⁶s on different carbon atoms together with the carbon atoms to which they are attached combine to form an oxirane, tetrahydrofuran, or tetrahydropyran.

In certain embodiments, the compound is selected from the group consisting of:

TABLE 6
Compounds of Formula IA-9a, IA-9b, IB-9a, IB-9b, IC-9a and IC-9b

| | |
|---|---|
| 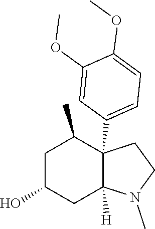 [SMILES: COc1ccc([C@]23CCN(C)[C@H]2C[C@H](O)C[C@H]3C)cc1OC] | 0901 |
| 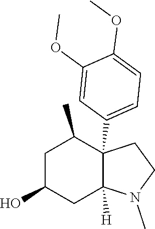 [SMILES: COc1ccc([C@]23CCN(C)[C@H]2C[C@@H](O)C[C@H]3C)cc1OC] | 0902 |
| 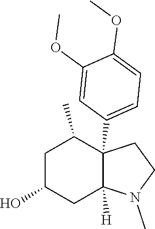 [SMILES: COc1ccc([C@]23CCN(C)[C@H]2C[C@H](O)C[C@@H]3C)cc1OC] | 0903 |
| 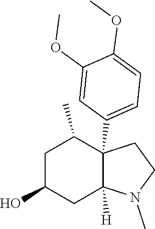 [SMILES: COc1ccc([C@]23CCN(C)[C@H]2C[C@@H](O)C[C@@H]3C)cc1OC] | 0904 |
| 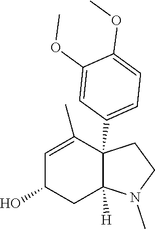 [SMILES: COc1ccc([C@]23CCN(C)[C@H]2C[C@H](O)C=C3C)cc1OC] | 0905 |
| 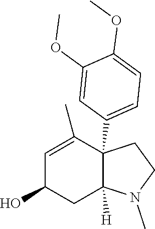 [SMILES: COc1ccc([C@]23CCN(C)[C@H]2C[C@@H](O)C=C3C)cc1OC] | 0906 |
| 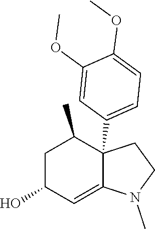 [SMILES: COc1ccc([C@]23CCN(C)C2=C[C@H](O)C[C@H]3C)cc1OC] | 0907 |
| 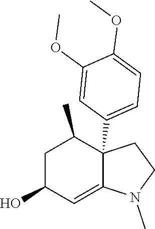 [SMILES: COc1ccc([C@]23CCN(C)C2=C[C@@H](O)C[C@H]3C)cc1OC] | 0908 |
| 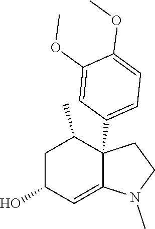 [SMILES: COc1ccc([C@]23CCN(C)C2=C[C@H](O)C[C@@H]3C)cc1OC] | 0909 |
| 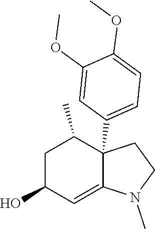 [SMILES: COc1ccc([C@]23CCN(C)C2=C[C@@H](O)C[C@@H]3C)cc1OC] | 0910 |
| 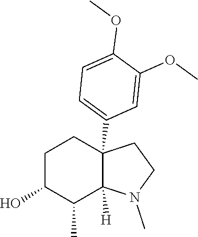 [SMILES: COc1ccc([C@@]23CC[C@@H](O)[C@@H](C)[C@@H]2N(C)CC3)cc1OC] | 0911 |
| 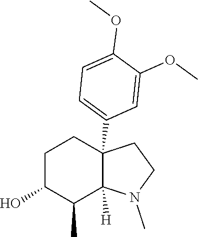 [SMILES: COc1ccc([C@@]23CC[C@@H](O)[C@H](C)[C@@H]2N(C)CC3)cc1OC] | 0912 |
| 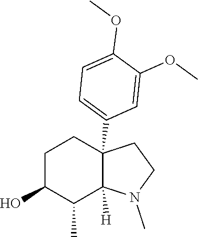 [SMILES: COc1ccc([C@@]23CC[C@H](O)[C@@H](C)[C@@H]2N(C)CC3)cc1OC] | 0913 |
| 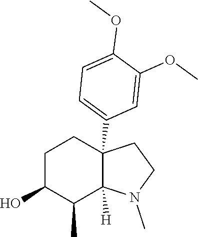 [SMILES: COc1ccc([C@@]23CC[C@H](O)[C@H](C)[C@@H]2N(C)CC3)cc1OC] | 0914 |
| 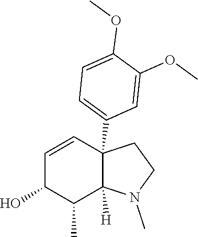 [SMILES: COc1ccc([C@@]23C=C[C@@H](O)[C@@H](C)[C@@H]2N(C)CC3)cc1OC] | 0915 |
| 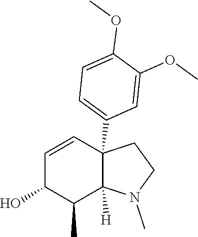 [SMILES: COc1ccc([C@@]23C=C[C@@H](O)[C@H](C)[C@@H]2N(C)CC3)cc1OC] | 0916 |
| 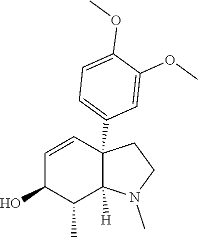 [SMILES: COc1ccc([C@@]23C=C[C@H](O)[C@@H](C)[C@@H]2N(C)CC3)cc1OC] | 0917 |
| 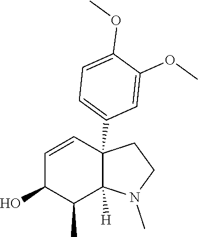 [SMILES: COc1ccc([C@@]23C=C[C@H](O)[C@H](C)[C@@H]2N(C)CC3)cc1OC] | 0918 |
| 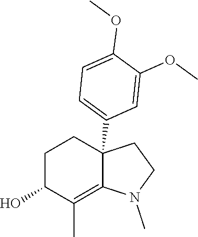 [SMILES: COc1ccc([C@@]23CC[C@@H](O)C(C)=C2N(C)CC3)cc1OC] | 0919 |
| 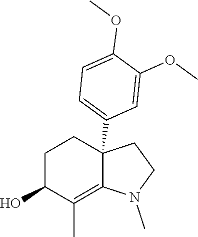 [SMILES: COc1ccc([C@@]23CC[C@H](O)C(C)=C2N(C)CC3)cc1OC] | 0920 |
| 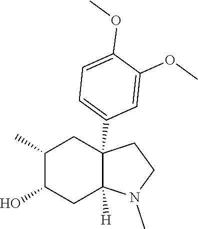 [SMILES: COc1ccc([C@]23CCN(C)[C@H]2C[C@H](O)[C@H](C)C3)cc1OC] | 0921 |
| 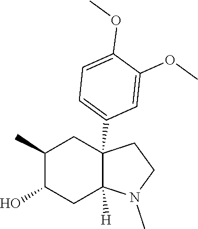 [SMILES: COc1ccc([C@]23CCN(C)[C@H]2C[C@H](O)[C@@H](C)C3)cc1OC] | 0922 |
| 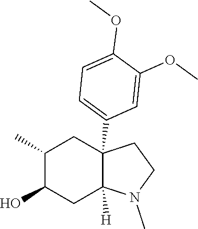 [SMILES: COc1ccc([C@]23CCN(C)[C@H]2C[C@@H](O)[C@H](C)C3)cc1OC] | 0923 |
| 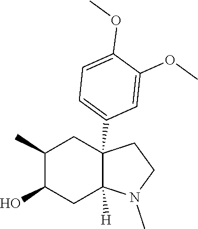 [SMILES: COc1ccc([C@]23CCN(C)[C@H]2C[C@@H](O)[C@@H](C)C3)cc1OC] | 0924 |
| 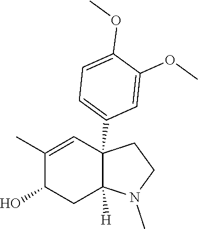 [SMILES: COc1ccc([C@@]23C=C(C)[C@@H](O)C[C@@H]2N(C)CC3)cc1OC] | 0925 |
| 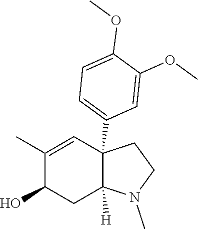 [SMILES: COc1ccc([C@@]23C=C(C)[C@H](O)C[C@@H]2N(C)CC3)cc1OC] | 0926 |
| 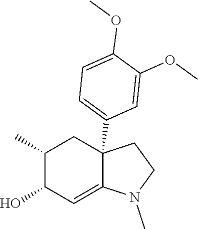 [SMILES: COc1ccc([C@]23CCN(C)C2=C[C@H](O)[C@H](C)C3)cc1OC] | 0927 |
| 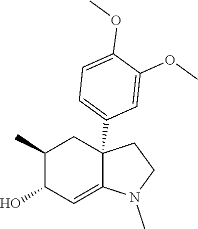 [SMILES: COc1ccc([C@]23CCN(C)C2=C[C@H](O)[C@@H](C)C3)cc1OC] | 0928 |
| 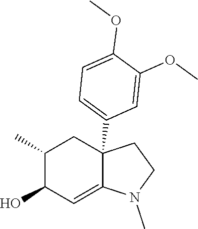 [SMILES: COc1ccc([C@]23CCN(C)C2=C[C@@H](O)[C@H](C)C3)cc1OC] | 0929 |
| 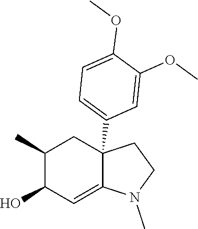 [SMILES: COc1ccc([C@]23CCN(C)C2=C[C@@H](O)[C@@H](C)C3)cc1OC] | 0930 |
| 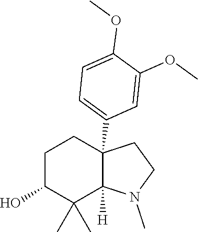 [SMILES: COc1ccc([C@@]23CC[C@@H](O)C(C)(C)[C@@H]2N(C)CC3)cc1OC] | 0931 |
| 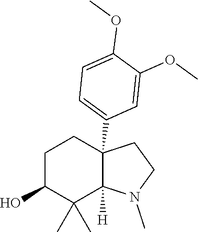 [SMILES: COc1ccc([C@@]23CC[C@H](O)C(C)(C)[C@@H]2N(C)CC3)cc1OC] | 0932 |
| 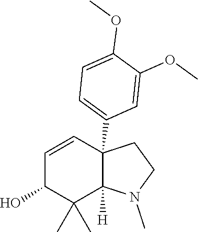 [SMILES: COc1ccc([C@@]23C=C[C@@H](O)C(C)(C)[C@@H]2N(C)CC3)cc1OC] | 0933 |
| 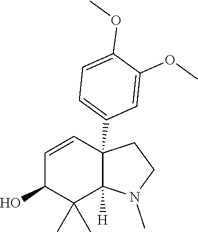 [SMILES: COc1ccc([C@@]23C=C[C@H](O)C(C)(C)[C@@H]2N(C)CC3)cc1OC] | 0934 |
| 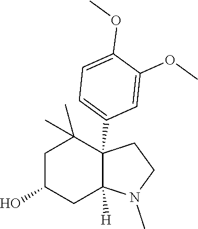 [SMILES: COc1ccc([C@]23CCN(C)[C@H]2C[C@H](O)CC3(C)C)cc1OC] | 0935 |
| 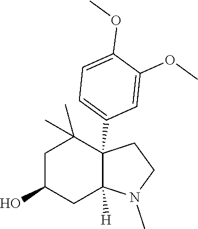 [SMILES: COc1ccc([C@]23CCN(C)[C@H]2C[C@@H](O)CC3(C)C)cc1OC] | 0936 |
| 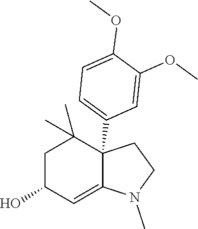 [SMILES: COc1ccc([C@]23CCN(C)C2=C[C@H](O)CC3(C)C)cc1OC] | 0937 |
| 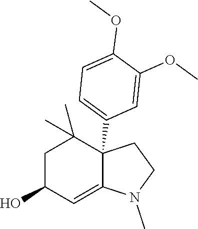 [SMILES: COc1ccc([C@]23CCN(C)C2=C[C@@H](O)CC3(C)C)cc1OC] | 0938 |
| 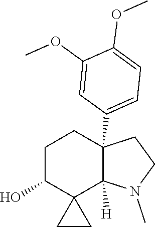 [SMILES: COc1ccc([C@@]23CC[C@@H](O)C4(CC4)[C@@H]2N(C)CC3)cc1OC] | 0939 |
| 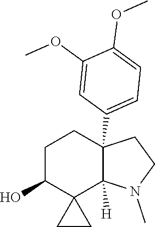 [SMILES: COc1ccc([C@@]23CC[C@H](O)C4(CC4)[C@@H]2N(C)CC3)cc1OC] | 0940 |
| 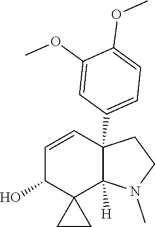 [SMILES: COc1ccc([C@@]23C=C[C@@H](O)C4(CC4)[C@@H]2N(C)CC3)cc1OC] | 0941 |
| 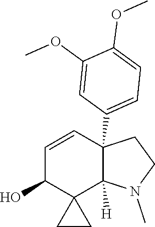 [SMILES: COc1ccc([C@@]23C=C[C@H](O)C4(CC4)[C@@H]2N(C)CC3)cc1OC] | 0942 |
| 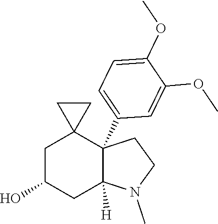 [SMILES: COc1ccc([C@]23CCN(C)[C@H]2C[C@H](O)CC32CC2)cc1OC] | 0943 |
| 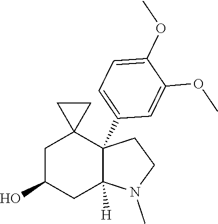 [SMILES: COc1ccc([C@]23CCN(C)[C@H]2C[C@@H](O)CC32CC2)cc1OC] | 0944 |
| 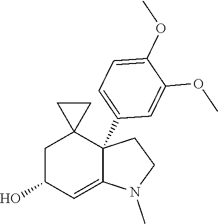 [SMILES: COc1ccc([C@]23CCN(C)C2=C[C@H](O)CC32CC2)cc1OC] | 0945 |
| 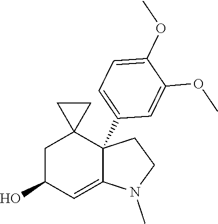 [SMILES: COc1ccc([C@]23CCN(C)C2=C[C@@H](O)CC32CC2)cc1OC] | 0946 |
| 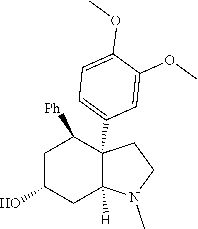 [SMILES: COc1ccc([C@]23CCN(C)[C@H]2C[C@H](O)C[C@H]3c2ccccc2)cc1OC] | 0947 |
| 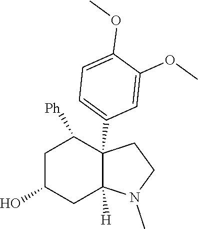 [SMILES: COc1ccc([C@]23CCN(C)[C@H]2C[C@H](O)C[C@@H]3c2ccccc2)cc1OC] | 0948 |
| 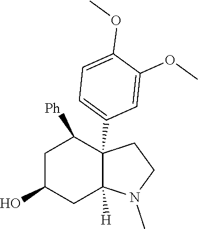 [SMILES: COc1ccc([C@]23CCN(C)[C@H]2C[C@@H](O)C[C@H]3c2ccccc2)cc1OC] | 0949 |
| 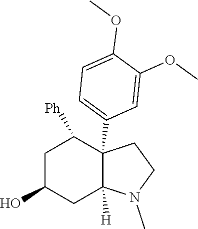 [SMILES: COc1ccc([C@]23CCN(C)[C@H]2C[C@@H](O)C[C@@H]3c2ccccc2)cc1OC] | 0950 |
| 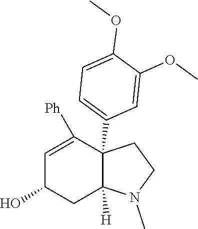 [SMILES: COc1ccc([C@]23CCN(C)[C@H]2C[C@H](O)C=C3c2ccccc2)cc1OC] | 0951 |
| 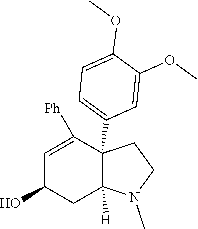 [SMILES: COc1ccc([C@]23CCN(C)[C@H]2C[C@@H](O)C=C3c2ccccc2)cc1OC] | 0952 |
| 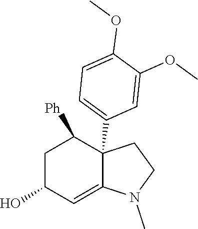 [SMILES: COc1ccc([C@]23CCN(C)C2=C[C@H](O)C[C@H]3c2ccccc2)cc1OC] | 0953 |
| 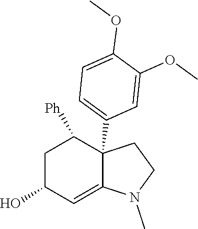 [SMILES: COc1ccc([C@]23CCN(C)C2=C[C@H](O)C[C@@H]3c2ccccc2)cc1OC] | 0954 |
| 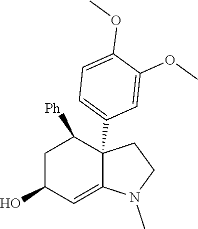 [SMILES: COc1ccc([C@]23CCN(C)C2=C[C@@H](O)C[C@H]3c2ccccc2)cc1OC] | 0955 |
| 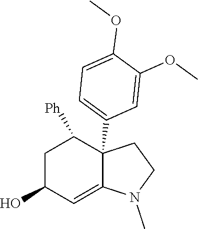 [SMILES: COc1ccc([C@]23CCN(C)C2=C[C@@H](O)C[C@@H]3c2ccccc2)cc1OC] | 0956 |
| 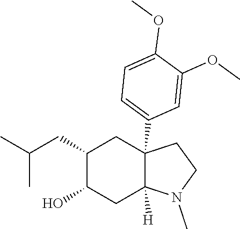 [SMILES: COc1ccc([C@]23CCN(C)[C@H]2C[C@H](O)[C@H](CC(C)C)C3)cc1OC] | 0957 |
| 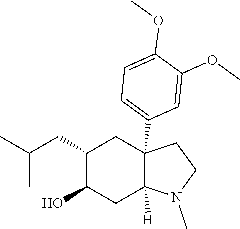 [SMILES: COc1ccc([C@]23CCN(C)[C@H]2C[C@@H](O)[C@H](CC(C)C)C3)cc1OC] | 0958 |
| 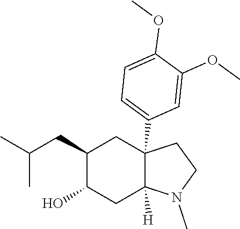 [SMILES: COc1ccc([C@]23CCN(C)[C@H]2C[C@H](O)[C@@H](CC(C)C)C3)cc1OC] | 0959 |
| 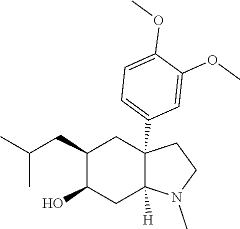 [SMILES: COc1ccc([C@]23CCN(C)[C@H]2C[C@@H](O)[C@@H](CC(C)C)C3)cc1OC] | 0960 |
| 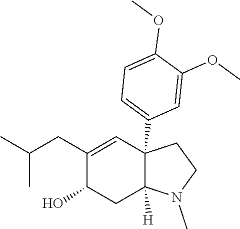 [SMILES: COc1ccc([C@@]23C=C(CC(C)C)[C@@H](O)C[C@@H]2N(C)CC3)cc1OC] | 0961 |
| 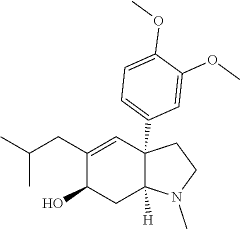 [SMILES: COc1ccc([C@@]23C=C(CC(C)C)[C@H](O)C[C@@H]2N(C)CC3)cc1OC] | 0962 |
| 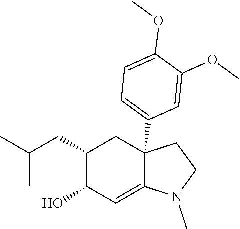 [SMILES: COc1ccc([C@]23CCN(C)C2=C[C@H](O)[C@H](CC(C)C)C3)cc1OC] | 0963 |
| 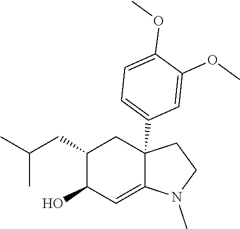 [SMILES: COc1ccc([C@]23CCN(C)C2=C[C@@H](O)[C@H](CC(C)C)C3)cc1OC] | 0964 |
| 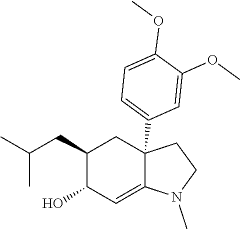 [SMILES: COc1ccc([C@]23CCN(C)C2=C[C@H](O)[C@@H](CC(C)C)C3)cc1OC] | 0965 |
| 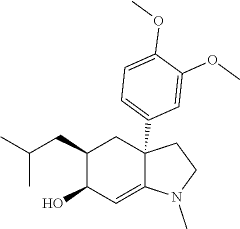 [SMILES: COc1ccc([C@]23CCN(C)C2=C[C@@H](O)[C@@H](CC(C)C)C3)cc1OC] | 0966 |
| 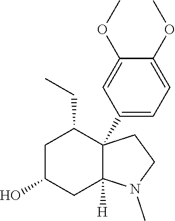 [SMILES: CC[C@H]1C[C@@H](O)C[C@@H]2N(C)CC[C@]12c1ccc(OC)c(OC)c1] | 0967 |
| 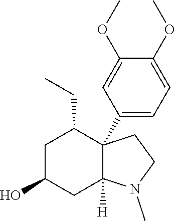 [SMILES: CC[C@H]1C[C@H](O)C[C@@H]2N(C)CC[C@]12c1ccc(OC)c(OC)c1] | 0968 |
| 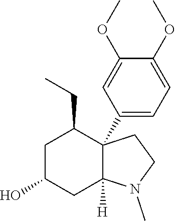 [SMILES: CC[C@@H]1C[C@@H](O)C[C@@H]2N(C)CC[C@]12c1ccc(OC)c(OC)c1] | 0969 |
| 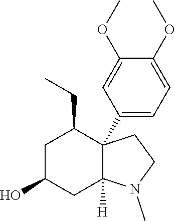 [SMILES: CC[C@@H]1C[C@H](O)C[C@@H]2N(C)CC[C@]12c1ccc(OC)c(OC)c1] | 0970 |
| 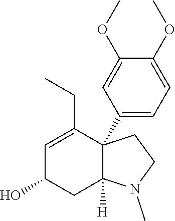 [SMILES: CCC1=C[C@@H](O)C[C@@H]2N(C)CC[C@]12c1ccc(OC)c(OC)c1] | 0971 |
| 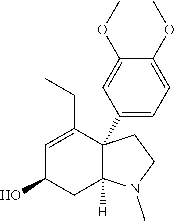 [SMILES: CCC1=C[C@H](O)C[C@@H]2N(C)CC[C@]12c1ccc(OC)c(OC)c1] | 0972 |
| 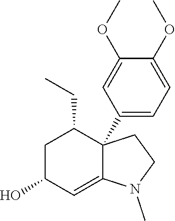 [SMILES: CC[C@H]1C[C@@H](O)C=C2N(C)CC[C@@]21c1ccc(OC)c(OC)c1] | 0973 |
| 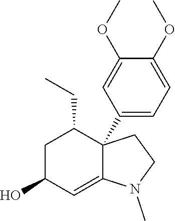 [SMILES: CC[C@H]1C[C@H](O)C=C2N(C)CC[C@@]21c1ccc(OC)c(OC)c1] | 0974 |
| 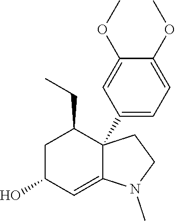 [SMILES: CC[C@@H]1C[C@@H](O)C=C2N(C)CC[C@@]21c1ccc(OC)c(OC)c1] | 0975 |
| 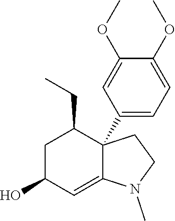 [SMILES: CC[C@@H]1C[C@H](O)C=C2N(C)CC[C@@]21c1ccc(OC)c(OC)c1] | 0976 |
| 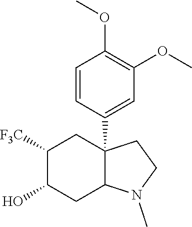 [SMILES: COc1ccc([C@]23CCN(C)C2C[C@H](O)[C@H](C(F)(F)F)C3)cc1OC] | 0977 |
| 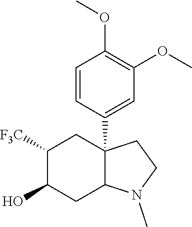 [SMILES: COc1ccc([C@]23CCN(C)C2C[C@@H](O)[C@H](C(F)(F)F)C3)cc1OC] | 0978 |
| 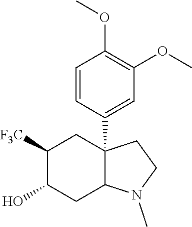 [SMILES: COc1ccc([C@]23CCN(C)C2C[C@H](O)[C@@H](C(F)(F)F)C3)cc1OC] | 0979 |
| 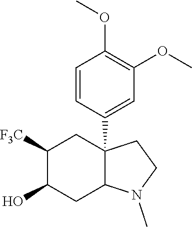 [SMILES: COc1ccc([C@]23CCN(C)C2C[C@@H](O)[C@@H](C(F)(F)F)C3)cc1OC] | 0980 |
| 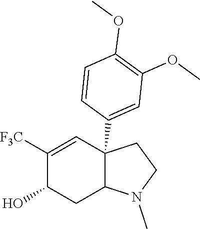 [SMILES: COc1ccc([C@@]23C=C(C(F)(F)F)[C@@H](O)CC2N(C)CC3)cc1OC] | 0981 |
| 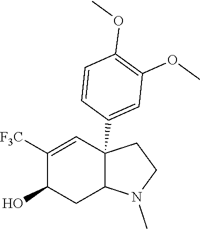 [SMILES: COc1ccc([C@@]23C=C(C(F)(F)F)[C@H](O)CC2N(C)CC3)cc1OC] | 0982 |
| 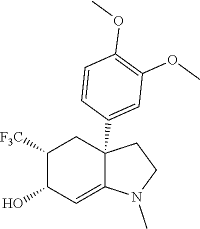 [SMILES: COc1ccc([C@]23CCN(C)C2=C[C@H](O)[C@H](C(F)(F)F)C3)cc1OC] | 0983 |
| 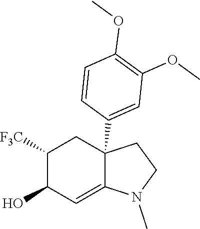 [SMILES: COc1ccc([C@]23CCN(C)C2=C[C@@H](O)[C@H](C(F)(F)F)C3)cc1OC] | 0984 |
| 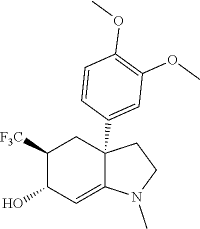 [SMILES: COc1ccc([C@]23CCN(C)C2=C[C@H](O)[C@@H](C(F)(F)F)C3)cc1OC] | 0985 |
| 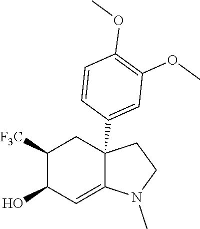 [SMILES: COc1ccc([C@]23CCN(C)C2=C[C@@H](O)[C@@H](C(F)(F)F)C3)cc1OC] | 0986 |
| 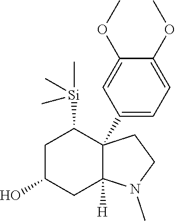 [SMILES: COc1ccc([C@]23CCN(C)[C@H]2C[C@H](O)C[C@@H]3[Si](C)(C)C)cc1OC] | 0987 |
| 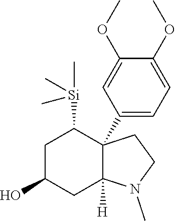 [SMILES: COc1ccc([C@]23CCN(C)[C@H]2C[C@@H](O)C[C@@H]3[Si](C)(C)C)cc1OC] | 0988 |
| 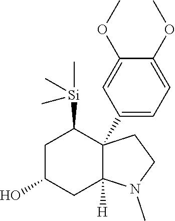 [SMILES: COc1ccc([C@]23CCN(C)[C@H]2C[C@H](O)C[C@H]3[Si](C)(C)C)cc1OC] | 0989 |
| 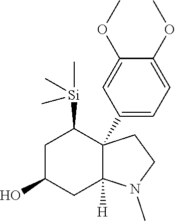 [SMILES: COc1ccc([C@]23CCN(C)[C@H]2C[C@@H](O)C[C@H]3[Si](C)(C)C)cc1OC] | 0990 |
| 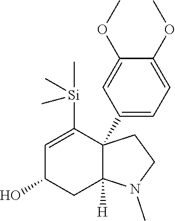 [SMILES: COc1ccc([C@]23CCN(C)[C@H]2C[C@H](O)C=C3[Si](C)(C)C)cc1OC] | 0991 |
| 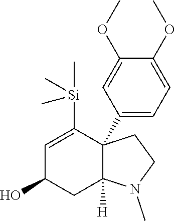 [SMILES: COc1ccc([C@]23CCN(C)[C@H]2C[C@@H](O)C=C3[Si](C)(C)C)cc1OC] | 0992 |
| 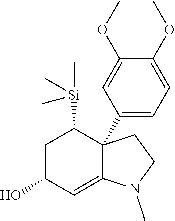 [SMILES: COc1ccc([C@]23CCN(C)C2=C[C@H](O)C[C@@H]3[Si](C)(C)C)cc1OC] | 0993 |
| 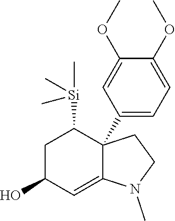 [SMILES: COc1ccc([C@]23CCN(C)C2=C[C@@H](O)C[C@@H]3[Si](C)(C)C)cc1OC] | 0994 |
| 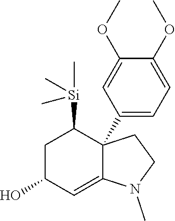 [SMILES: COc1ccc([C@]23CCN(C)C2=C[C@H](O)C[C@H]3[Si](C)(C)C)cc1OC] | 0995 |
| 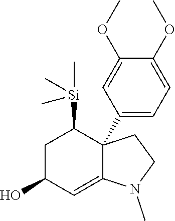 [SMILES: COc1ccc([C@]23CCN(C)C2=C[C@@H](O)C[C@H]3[Si](C)(C)C)cc1OC] | 0996 |
| 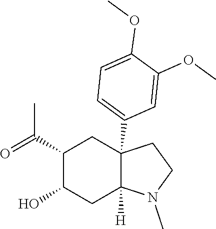 [SMILES: COc1ccc([C@]23CCN(C)[C@H]2C[C@H](O)[C@H](C(C)=O)C3)cc1OC] | 0997 |
| 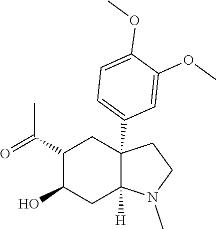 | 0998 |
| 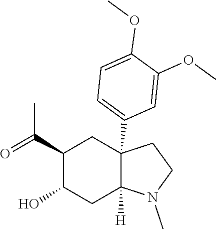 [SMILES: COc1ccc([C@]23CCN(C)[C@H]2C[C@H](O)[C@@H](C(C)=O)C3)cc1OC] | 0999 |
| 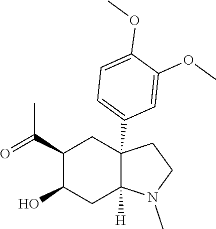 [SMILES: COc1ccc([C@]23CCN(C)[C@H]2C[C@@H](O)[C@@H](C(C)=O)C3)cc1OC] | 1900 |
| 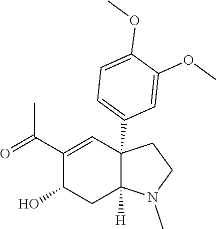 [SMILES: COc1ccc([C@@]23C=C(C(C)=O)[C@@H](O)C[C@@H]2N(C)CC3)cc1OC] | 1901 |
| 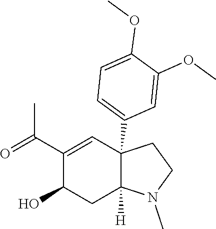 [SMILES: COc1ccc([C@@]23C=C(C(C)=O)[C@H](O)C[C@@H]2N(C)CC3)cc1OC] | 1902 |
| 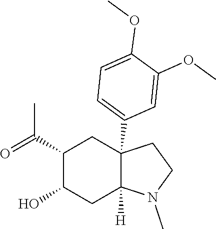 [SMILES: COc1ccc([C@]23CCN(C)[C@H]2C[C@H](O)[C@H](C(C)=O)C3)cc1OC] | 1903 |
| 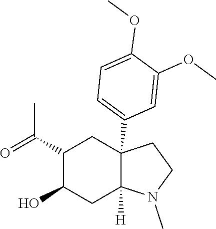 | 1904 |
| 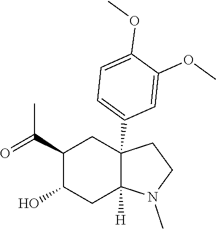 [SMILES: COc1ccc([C@]23CCN(C)[C@H]2C[C@H](O)[C@@H](C(C)=O)C3)cc1OC] | 1905 |
| 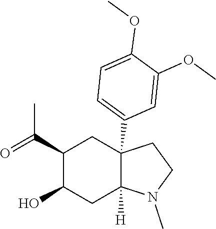 [SMILES: COc1ccc([C@]23CCN(C)[C@H]2C[C@@H](O)[C@@H](C(C)=O)C3)cc1OC] | 1906 |
| 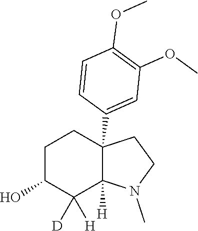 [SMILES: [2H]C1[C@H](O)CC[C@@]2(c3ccc(OC)c(OC)c3)CCN(C)[C@@H]12] | 1907 |
| 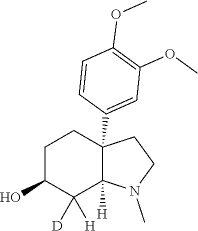 [SMILES: [2H]C1[C@@H]2N(C)CC[C@]2(c2ccc(OC)c(OC)c2)CC[C@@H]1O] | 1908 |
| 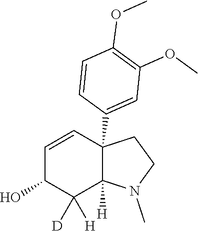 [SMILES: [2H]C1[C@H](O)C=C[C@@]2(c3ccc(OC)c(OC)c3)CCN(C)[C@@H]12] | 1909 |
| 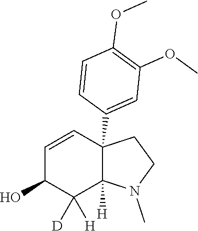 [SMILES: [2H]C1[C@@H]2N(C)CC[C@]2(c2ccc(OC)c(OC)c2)C=C[C@@H]1O] | 1910 |
| 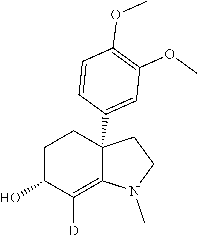 [SMILES: [2H]C1=C2N(C)CC[C@]2(c2ccc(OC)c(OC)c2)CC[C@H]1O] | 1911 |
| 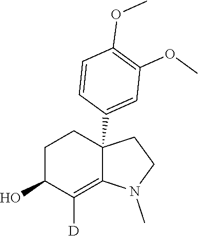 [SMILES: [2H]C1=C2N(C)CC[C@]2(c2ccc(OC)c(OC)c2)CC[C@@H]1O] | 1912 |
| 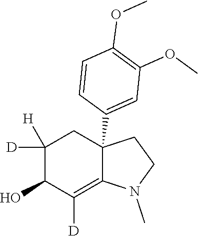 [SMILES: [2H]C1=C2N(C)CC[C@]2(c2ccc(OC)c(OC)c2)CC([2H])[C@@H]1O] | 1913 |
| 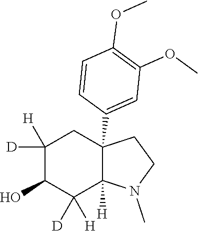 [SMILES: [2H]C1C[C@@]2(c3ccc(OC)c(OC)c3)CCN(C)[C@H]2C([2H])[C@H]1O] | 1914 |
| 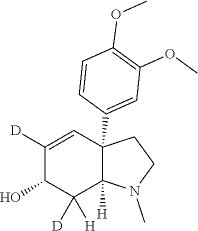 [SMILES: [2H]C1=C[C@@]2(c3ccc(OC)c(OC)c3)CCN(C)[C@H]2C([2H])[C@@H]1O] | 1915 |
| 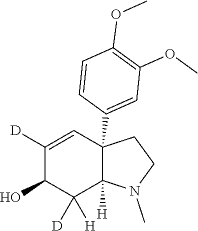 [SMILES: [2H]C1=C[C@@]2(c3ccc(OC)c(OC)c3)CCN(C)[C@H]2C([2H])[C@H]1O] | 1916 |
| 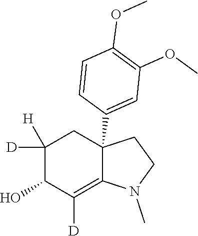 [SMILES: [2H]C1=C2N(C)CC[C@]2(c2ccc(OC)c(OC)c2)CC([2H])[C@H]1O] | 1917 |
| 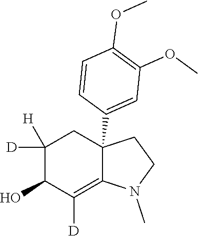 [SMILES: [2H]C1=C2N(C)CC[C@]2(c2ccc(OC)c(OC)c2)CC([2H])[C@@H]1O] | 1918 |
| 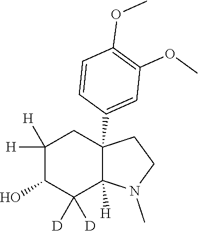 [SMILES: [2H]C1([2H])[C@H](O)CC[C@@]2(c3ccc(OC)c(OC)c3)CCN(C)[C@@H]12] | 1919 |
| 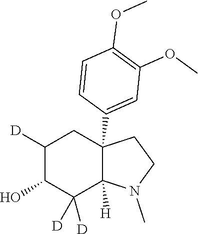 [SMILES: [2H]C1C[C@@]2(c3ccc(OC)c(OC)c3)CCN(C)[C@H]2C([2H])([2H])[C@@H]1O] | 1920 |
| 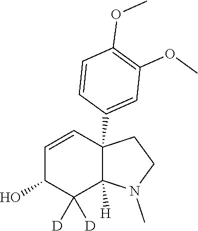 [SMILES: [2H]C1([2H])[C@H](O)C=C[C@@]2(c3ccc(OC)c(OC)c3)CCN(C)[C@@H]12] | 1921 |
| 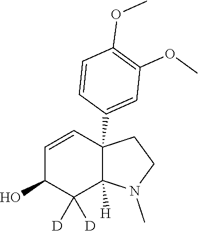 [SMILES: [2H]C1([2H])[C@@H](O)C=C[C@@]2(c3ccc(OC)c(OC)c3)CCN(C)[C@@H]12] | 1922 |
| 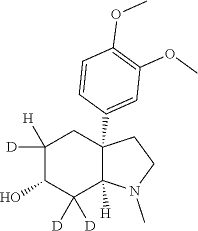 [SMILES: [2H]C1C[C@@]2(c3ccc(OC)c(OC)c3)CCN(C)[C@H]2C([2H])([2H])[C@@H]1O] | 1923 |
| 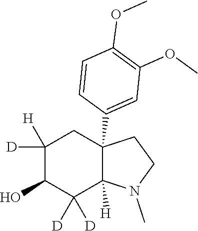 [SMILES: [2H]C1C[C@@]2(c3ccc(OC)c(OC)c3)CCN(C)[C@H]2C([2H])([2H])[C@H]1O] | 1924 |
| 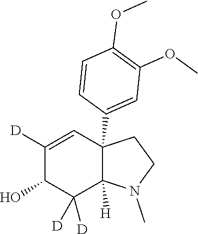 [SMILES: [2H]C1=C[C@@]2(c3ccc(OC)c(OC)c3)CCN(C)[C@H]2C([2H])([2H])[C@@H]1O] | 1925 |
| 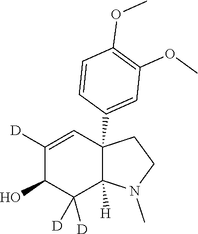 [SMILES: [2H]C1=C[C@@]2(c3ccc(OC)c(OC)c3)CCN(C)[C@H]2C([2H])([2H])[C@H]1O] | 1926 |
| 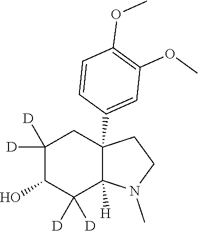 [SMILES: [2H]C1([2H])C[C@@]2(c3ccc(OC)c(OC)c3)CCN(C)[C@H]2C([2H])([2H])[C@@H]1O] | 1927 |
| 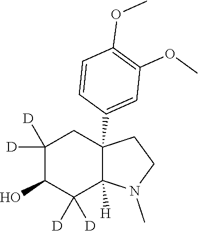 [SMILES: [2H]C1([2H])C[C@@]2(c3ccc(OC)c(OC)c3)CCN(C)[C@H]2C([2H])([2H])[C@H]1O] | 1928 |
| 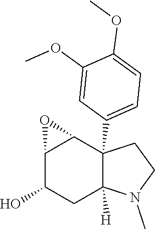 [SMILES: COc1ccc([C@]23CCN(C)[C@H]2C[C@H](O)[C@@H]2O[C@@H]23)cc1OC] | 1929 |
| 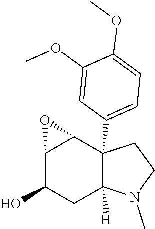 [SMILES: COc1ccc([C@]23CCN(C)[C@H]2C[C@@H](O)[C@@H]2O[C@@H]23)cc1OC] | 1930 |
| 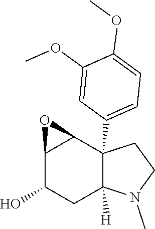 [SMILES: COc1ccc([C@]23CCN(C)[C@H]2C[C@H](O)[C@H]2O[C@H]23)cc1OC] | 1931 |
| 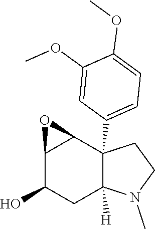 [SMILES: COc1ccc([C@]23CCN(C)[C@H]2C[C@@H](O)[C@H]2O[C@H]23)cc1OC] | 1932 |
| 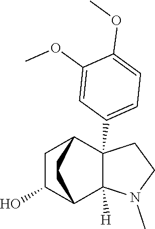 [SMILES: COc1ccc([C@]23CCN(C)[C@H]2[C@H]2CC[C@@H]3C[C@H]2O)cc1OC] | 1933 |
| 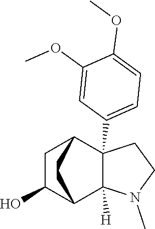 [SMILES: COc1ccc([C@]23CCN(C)[C@H]2[C@H]2CC[C@@H]3C[C@@H]2O)cc1OC] | 1934 |
| 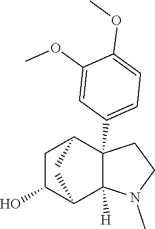 | 1935 |
| 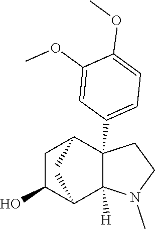 | 1936 |
| 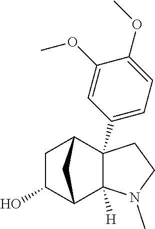 [SMILES: COc1ccc([C@]23CCN(C)[C@H]2[C@H]2C[C@@H]3C[C@H]2O)cc1OC] | 1937 |
| 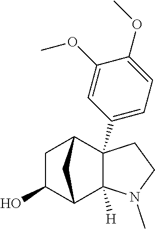 [SMILES: COc1ccc([C@]23CCN(C)[C@H]2[C@H]2C[C@@H]3C[C@@H]2O)cc1OC] | 1938 |
| 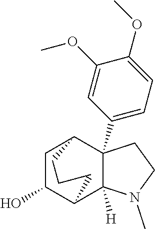 [SMILES: COc1ccc([C@]23CCN(C)[C@H]2[C@@H]2CCC[C@H]3C[C@H]2O)cc1OC] | 1939 |
| 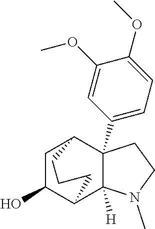 [SMILES: COc1ccc([C@]23CCN(C)[C@H]2[C@@H]2CCC[C@H]3C[C@@H]2O)cc1OC] | 1940 |
| 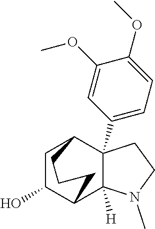 [SMILES: COc1ccc([C@]23CCN(C)[C@H]2[C@H]2CCC[C@@H]3C[C@H]2O)cc1OC] | 1941 |
| 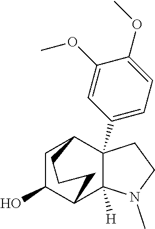 [SMILES: COc1ccc([C@]23CCN(C)[C@H]2[C@H]2CCC[C@@H]3C[C@@H]2O)cc1OC] | 1942 |

or a pharmaceutically acceptable salt thereof.

In certain embodiments, the compound is of Formula (IA-10), (IB-10), or (IC-10):

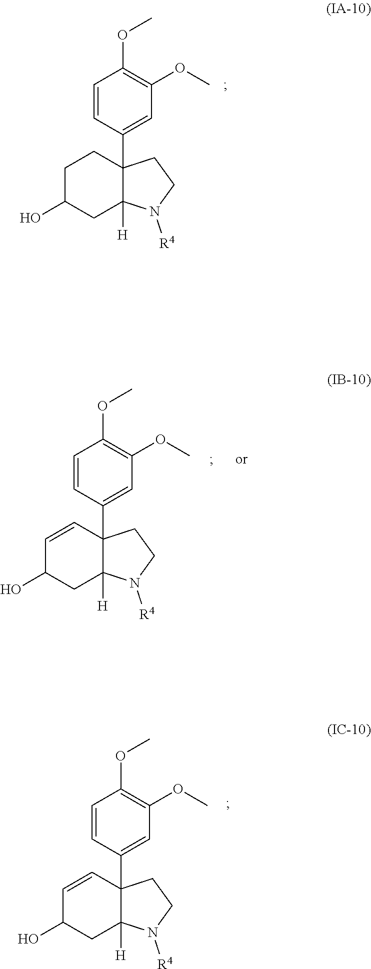

or a pharmaceutically acceptable salt thereof.

In certain embodiments, the compound is of Formula (IA-10) or a pharmaceutically acceptable salt thereof.

In certain embodiments, the compound is of Formula (IB-10) or a pharmaceutically acceptable salt thereof.

In certain embodiments, the compound is of Formula (IC-10) or a pharmaceutically acceptable salt thereof.

In certain embodiments, the compound is of Formula (IA-10a), (IA-10b), (IB-10a), (IB-10b), (IC-10a) or (IC-10b):

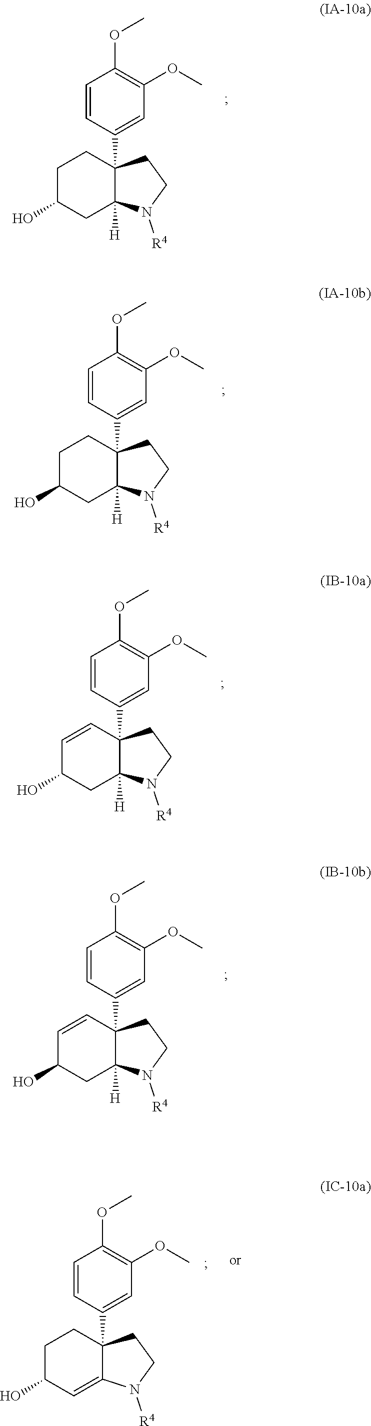

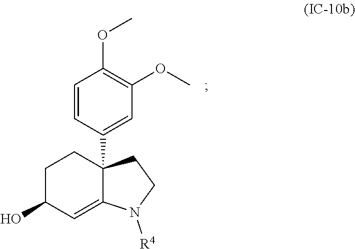

or a pharmaceutically acceptable salt thereof.

In certain embodiments, the compounds of Formula (IA-10a), (IA-10b), (IB-10a), (IB-10b), (IC-10a) or (IC-10b) have the absolute stereochemistry shown.

In certain embodiments, the compound is of Formula (IA-10a) or a pharmaceutically acceptable salt thereof.

In certain embodiments, the compound is of Formula (IA-10b) or a pharmaceutically acceptable salt thereof.

In certain embodiments, the compound is of Formula (IB-10a) or a pharmaceutically acceptable salt thereof.

In certain embodiments, the compound is of Formula (IB-10b) or a pharmaceutically acceptable salt thereof.

In certain embodiments, the compound is of Formula (IC-10a) or a pharmaceutically acceptable salt thereof.

In certain embodiments, the compound is of Formula (IC-10b) or a pharmaceutically acceptable salt thereof.

In certain embodiments, R⁴ is H or C₁-C₆ alkyl optionally substituted by deuterium, phenyl optionally substituted by —OC₁-C₆ alkyl, or C₃-C₆ cycloalkyl. For example, in certain embodiments, R⁴ is methyl, ethyl, or CD₃.

In certain embodiments, the compound is selected from the group consisting of:

TABLE 7
Compounds of Formula IA-10a, IA-10b, IB-10a, IB-10b,
IC-10a, and IC-10b

| | |
|---|---|
| 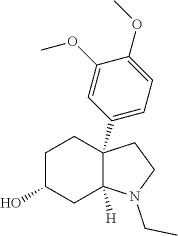 [SMILES: CCN1CC[C@]2(c3ccc(OC)c(OC)c3)CC[C@@H](O)C[C@H]12] | 1001 |
| 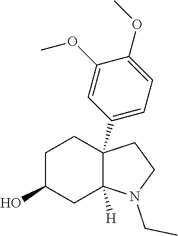 [SMILES: CCN1CC[C@]2(c3ccc(OC)c(OC)c3)CC[C@H](O)C[C@H]12] | 1002 |
| 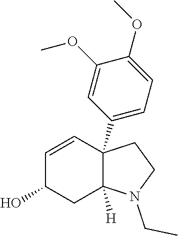 [SMILES: CCN1CC[C@]2(c3ccc(OC)c(OC)c3)C=C[C@@H](O)C[C@H]12] | 1003 |
| 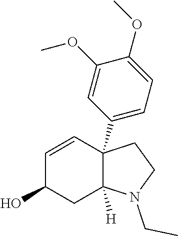 [SMILES: CCN1CC[C@]2(c3ccc(OC)c(OC)c3)C=C[C@H](O)C[C@H]12] | 1004 |
| 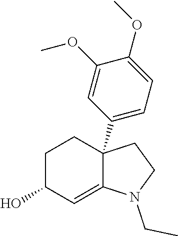 [SMILES: CCN1CC[C@]2(c3ccc(OC)c(OC)c3)CC[C@@H](O)C=C12] | 1005 |
| 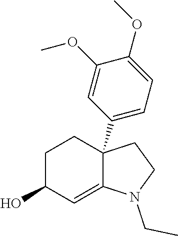 [SMILES: CCN1CC[C@]2(c3ccc(OC)c(OC)c3)CC[C@H](O)C=C12] | 1006 |
| 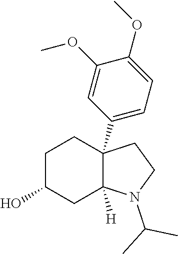 [SMILES: COc1ccc([C@@]23CC[C@@H](O)C[C@@H]2N(C(C)C)CC3)cc1OC] | 1007 |
| 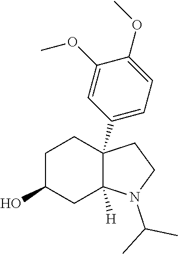 [SMILES: COc1ccc([C@@]23CC[C@H](O)C[C@@H]2N(C(C)C)CC3)cc1OC] | 1008 |
| 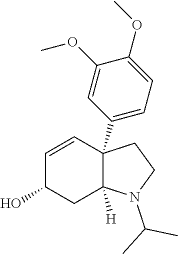 [SMILES: COc1ccc([C@@]23C=C[C@@H](O)C[C@@H]2N(C(C)C)CC3)cc1OC] | 1009 |
| 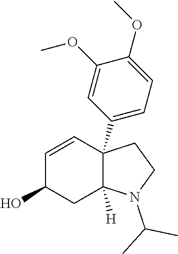 [SMILES: COc1ccc([C@@]23C=C[C@H](O)C[C@@H]2N(C(C)C)CC3)cc1OC] | 1010 |
| 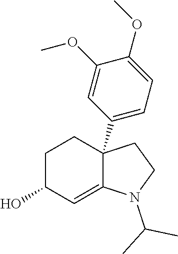 [SMILES: COc1ccc([C@@]23CC[C@@H](O)C=C2N(C(C)C)CC3)cc1OC] | 1011 |
| 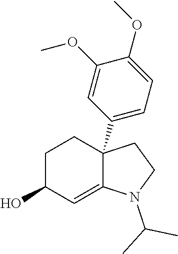 [SMILES: COc1ccc([C@@]23CC[C@H](O)C=C2N(C(C)C)CC3)cc1OC] | 1012 |
| 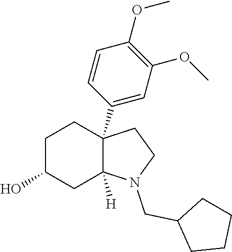 [SMILES: COc1ccc([C@@]23CC[C@@H](O)C[C@@H]2N(CC2CCCC2)CC3)cc1OC] | 1013 |
| 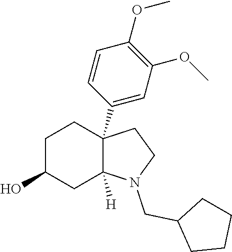 [SMILES: COc1ccc([C@@]23CC[C@H](O)C[C@@H]2N(CC2CCCC2)CC3)cc1OC] | 1014 |
| 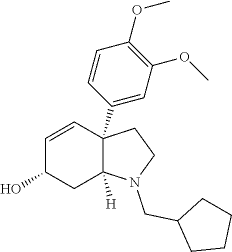 [SMILES: COc1ccc([C@@]23C=C[C@@H](O)C[C@@H]2N(CC2CCCC2)CC3)cc1OC] | 1015 |
| 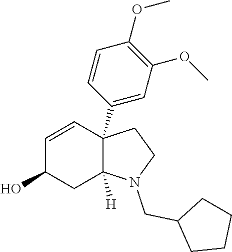 [SMILES: COc1ccc([C@@]23C=C[C@H](O)C[C@@H]2N(CC2CCCC2)CC3)cc1OC] | 1016 |
| 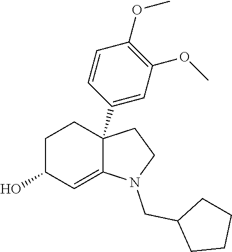 [SMILES: COc1ccc([C@@]23CC[C@@H](O)C=C2N(CC2CCCC2)CC3)cc1OC] | 1017 |
| 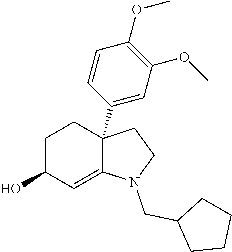 [SMILES: COc1ccc([C@@]23CC[C@H](O)C=C2N(CC2CCCC2)CC3)cc1OC] | 1018 |
| 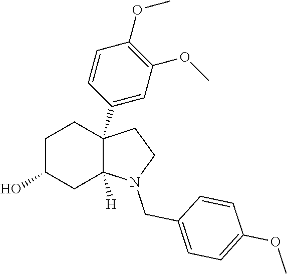 [SMILES: COc1ccc(CN2CC[C@]3(c4ccc(OC)c(OC)c4)CC[C@@H](O)C[C@H]23)cc1] | 1019 |
| 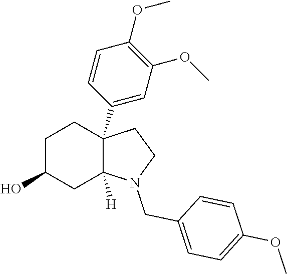 [SMILES: COc1ccc(CN2CC[C@]3(c4ccc(OC)c(OC)c4)CC[C@H](O)C[C@H]23)cc1] | 1020 |
| 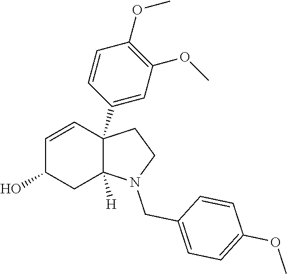 [SMILES: COc1ccc(CN2CC[C@]3(c4ccc(OC)c(OC)c4)C=C[C@@H](O)C[C@H]23)cc1] | 1021 |
| 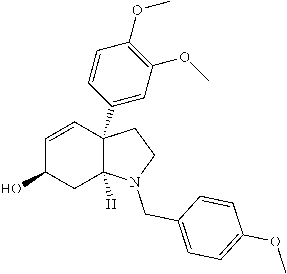 [SMILES: COc1ccc(CN2CC[C@]3(c4ccc(OC)c(OC)c4)C=C[C@H](O)C[C@H]23)cc1] | 1022 |
| 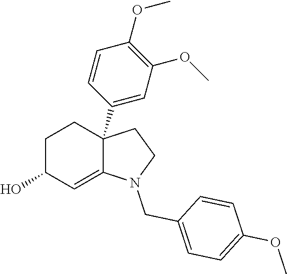 [SMILES: COc1ccc(CN2CC[C@]3(c4ccc(OC)c(OC)c4)CC[C@@H](O)C=C23)cc1] | 1023 |
| 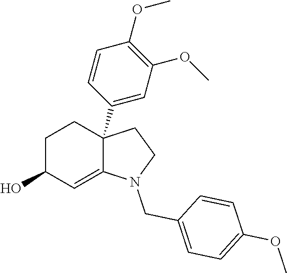 [SMILES: COc1ccc(CN2CC[C@]3(c4ccc(OC)c(OC)c4)CC[C@H](O)C=C23)cc1] | 1024 |
| 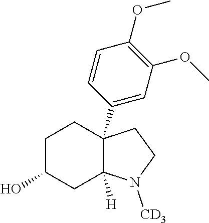 [SMILES: [2H]C([2H])([2H])N1CC[C@]2(c3ccc(OC)c(OC)c3)CC[C@@H](O)C[C@H]12] | 1025 |
| 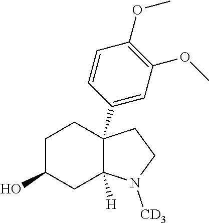 [SMILES: [2H]C([2H])([2H])N1CC[C@]2(c3ccc(OC)c(OC)c3)CC[C@H](O)C[C@H]12] | 1026 |
| 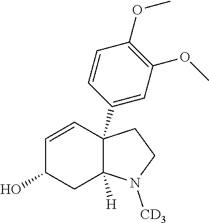 [SMILES: [2H]C([2H])([2H])N1CC[C@]2(c3ccc(OC)c(OC)c3)C=C[C@@H](O)C[C@H]12] | 1027 |
| 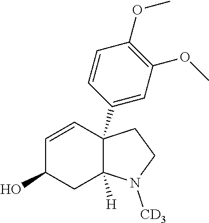 [SMILES: [2H]C([2H])([2H])N1CC[C@]2(c3ccc(OC)c(OC)c3)C=C[C@H](O)C[C@H]12] | 1028 |
| 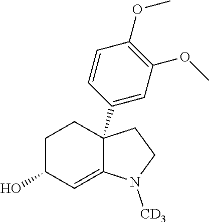 [SMILES: [2H]C([2H])([2H])N1CC[C@]2(c3ccc(OC)c(OC)c3)CC[C@@H](O)C=C12] | 1029 |
| 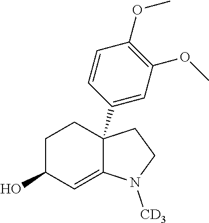 [SMILES: [2H]C([2H])([2H])N1CC[C@]2(c3ccc(OC)c(OC)c3)CC[C@H](O)C=C12] | 1030 |

or a pharmaceutically acceptable salt thereof.

In some embodiments, the compound is a compound of Formula (IA-c), (IB-c) or (IC-c):

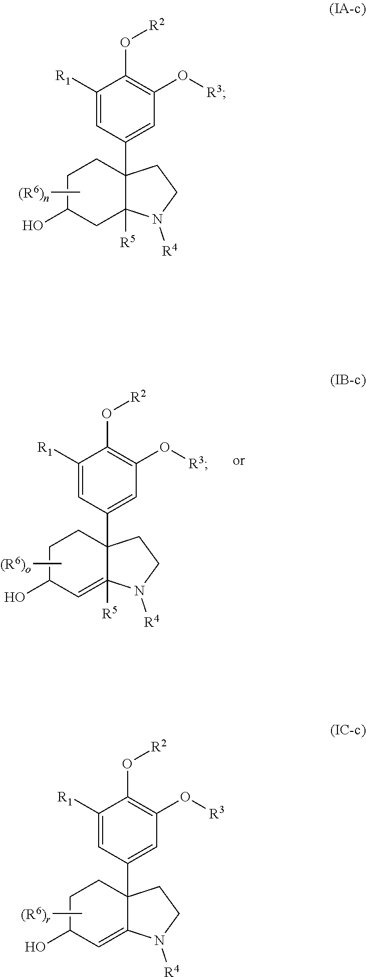

or a pharmaceutically acceptable salt thereof, wherein

R¹ is deuterium, halo, C₁-C₄ alkyl, C₂-C₄ alkenyl, C₂-C₄ alkynyl, C₃-C₆ cycloalkyl optionally substituted with C₂-C₄ alkenyl, 3- to 6-membered heterocyclyl, C₅-C₆ aryl, 5- to 6-membered heteroaryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, C₁-C₄ alkyl, C₁-C₄ alkanol, C₅-C₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R⁴ is H or alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a};
R⁵ is H, deuterium, or methyl;
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form cycloalkyl, heterocyclyl or heteroaryl, wherein each hydrogen atom in cycloalkyl, heterocyclyl, and heteroaryl is optionally substituted by halo, hydroxy, alkoxy, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b};
n is 0, 1, 2, 3, 4, 5, or 6;
o is 0, 1, 2, 3, or 4;
r is 0, 1, 2, 3, 4, or 5;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl;
provided the compound is not:

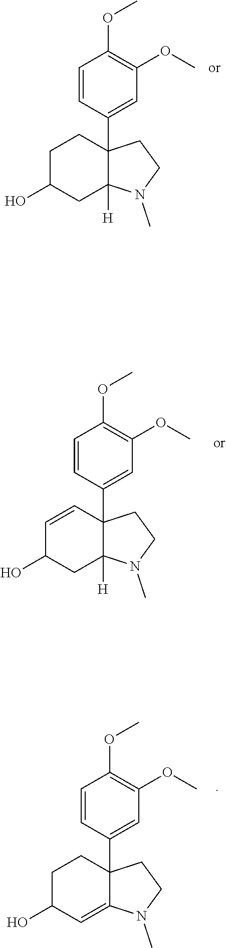 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

In some embodiments, the compound is a compound of Formula (IA-c), (IB-c) or (IC-c), wherein n is 0, o is 0 and r is 0.

In some embodiments, the compound is a compound of Formula (IA-c), (IB-c) or (IC-c), wherein R⁴ is hydrogen and R⁵ is methyl. In some embodiments, the compound is a compound of Formula (IA-c), (IB-c) or (IC-c), wherein R⁴ is hydrogen, R⁵ is methyl, and n, o, and r are each independently 0 or 1.

In some embodiments, the compound is a compound of Formula (IA-c), (IB-c) or (IC-c), wherein n, o, and r are each independently 0 or 1; R¹ is deuterium, halo, C₁-C₄ alkyl optionally substituted with halo or deuterium, C₂ alkenyl, C₃-C₆ cycloalkyl, —OR^{a}, —C(O)R^{a}, —C(O)NR^{a}R^{b}, or —CN; and R^{a} is C₁-C₄ alkyl. In some embodiments, the compound is a compound of Formula (IA-c), (IB-c) or (IC-c), wherein R¹ is deuterium, halo, C₁-C₄ alkyl optionally substituted with halo or deuterium, C₂-C₃ alkenyl, C₃-C₆ cycloalkyl, —OR^{a}, —C(O)R^{a}, —C(O)NR^{a}R^{b}, or —CN; and each of R^{a} and R^{b} is hydrogen or C₁-C₄ alkyl. In some embodiments, the compound is a compound of Formula (IA-c), (IB-c) or (IC-c), wherein n, o, and r are each independently 0 or 1; R¹ is deuterium, F, C₁-C₄ alkyl optionally substituted with F, C₂ alkenyl, cyclopropyl, methoxy, acetyl, —C(O)NH₂, or —CN. In some embodiments, the compound is a compound of Formula (IA-c), (IB-c) or (IC-c), wherein n, o, and r are each independently 0 or 1; R¹ is deuterium, F, C₁-C₄ alkyl optionally substituted with F, C₂ alkenyl, cyclopropyl, methoxy, acetyl, —C(O)NH₂, or —CN; R⁴ is hydrogen; and R⁵ is methyl.

In some embodiments, the compound is a compound of Formula (IA-c), (IB-c) or (IC-c), wherein each of R² and R³ is independently H, C₁-C₄ alkyl, C₃-C₆ cycloalkyl, C₂-C₃ alkenyl, C₂-C₃ alkynyl, or C₅-C₆ aryl; wherein each hydrogen atom in alkyl cycloalkyl, alkenyl, alkynyl, and aryl is optionally substituted by halo, deuterium, C₃-C₆ cycloalkyl, C₅-C₆ aryl, or OR^{a}; and R^{a} is C₁-C₄ alkyl. In some embodiments, the compound is a compound of Formula (IA-c), (IB-c) or (IC-c), wherein each of R² and R³ is independently C₁-C₄ alkyl optionally substituted with halo.

In some embodiments, the compound is a compound of Formula (IA-c), (IB-c) or (IC-c), wherein each of R² and R³ is independently C₁-C₄ alkyl optionally substituted with fluoro or deuterium. In some embodiments, the compound is a compound of Formula (IA-c), (IB-c) or (IC-c), wherein each of R² and R³ is independently C₁-C₄ alkyl. In some embodiments, the compound is a compound of Formula (IA-c), (IB-c) or (IC-c), wherein each of R² and R³ is independently methyl. In some embodiments, the compound is a compound of Formula (IA-c), (IB-c) or (IC-c), wherein R² is methyl and R³ is C₁-C₄ alkyl. In some embodiments, the compound is a compound of Formula (IA-c), (IB-c) or (IC-c), wherein R² is methyl and R³ is propyl. In some embodiments, the compound is a compound of Formula (IA-c), (IB-c) or (IC-c), wherein R² is methyl and R³ is butyl. In some embodiments, the compound is a compound of Formula (IA-c), (IB-c) or (IC-c), wherein each of R² and R³ is independently methyl, —CF₃ or CHF₂. In some embodiments, the compound is a compound of Formula (IA-c), (IB-c) or (IC-c), wherein each of R² is methyl and R³ is methyl, —CF₃ or CHF₂. In some embodiments, the compound is a compound of Formula (IA-c), (IB-c) or (IC-c), wherein each of R³ is methyl and R² is methyl, —CF₃ or CHF₂. In some embodiments, the compound is a compound of Formula (IA-c), (IB-c) or (IC-c), wherein each of R² is methyl and R³ is —CH₂CF₃ or —CH₂CHF₂. In some embodiments, the compound is a compound of Formula (IA-c), (IB-c) or (IC-c), wherein each of R³ is methyl and R² is —CH₂CF₃ or —CH₂CHF₂. In some embodiments, the compound is a compound of Formula (IA-c), (IB-c) or (IC-c), wherein each of R² and R³ is independently methyl, or —CD₃. In some embodiments, the compound is a compound of Formula (IA-c), (IB-c) or (IC-c), wherein each of R² is methyl and R³ is —CD₃. In some embodiments, the compound is a compound of Formula (IA-c), (IB-c) or (IC-c), wherein each of R³ is methyl and R² is —CD₃. In some embodiments, the compound is a compound of Formula (IA-c), (IB-c) or (IC-c), wherein each of R² and R³ is independently —CD₃.

In some embodiments, the compound is a compound of Formula (IA-c), (IB-c) or (IC-c), wherein each of R² and R³ is independently C₁-C₄ alkyl or C₁-C₄ alkyl substituted with C₃-C₆ cycloalkyl. In some embodiments, the compound is a compound of Formula (IA-c), (IB-c) or (IC-c), wherein each of R² and R³ is independently methyl or methyl substituted with C₃-C₆ cycloalkyl. In some embodiments, the compound is a compound of Formula (IA-c), (IB-c) or (IC-c), wherein each of R² and R³ is independently methyl or methyl substituted with cyclopropyl. In some embodiments, the compound is a compound of Formula (IA-c), (IB-c) or (IC-c), wherein R² is C₁-C₄ alkyl and R³ is methyl substituted with cyclopropyl. In some embodiments, the compound is a compound of Formula (IA-c), (IB-c) or (IC-c), wherein R³ is C₁-C₄ alkyl and R² is methyl substituted with cyclopropyl. In some embodiments, the compound is a compound of Formula (IA-c), (IB-c) or (IC-c), wherein R² is methyl and R³ is methyl substituted with cyclopropyl. In some embodiments, the compound is a compound of Formula (IA-c), (IB-c) or (IC-c), wherein R³ is methyl and R² is methyl substituted with cyclopropyl.

In some embodiments, the compound is a compound of Formula (IA-c), (IB-c) or (IC-c), wherein each of R² and R³ is independently C₁-C₄ alkyl or C₁-C₄ alkyl substituted with —OR^{a}, and R^{a} is C₁-C₄ alkyl. In some embodiments, the compound is a compound of Formula (IA-c), (IB-c) or (IC-c), wherein each of R² and R³ is independently methyl or methyl substituted with —OR^{a}, and R^{a} is C₁-C₄ alkyl. In some embodiments, the compound is a compound of Formula (IA-c), (IB-c) or (IC-c), wherein R² is methyl and R³ is methyl or —CH₂—O—CH₃.

In some embodiments, the compound is a compound of Formula (IA-c), (IB-c) or (IC-c), wherein each of R² and R³ is independently C₁-C₄ alkyl or C₃-C₆ cycloalkyl. In some embodiments, the compound is a compound of Formula (IA-c), (IB-c) or (IC-c), wherein each of R² and R³ is independently C₁-C₄ alkyl or C₄-C₆ cycloalkyl. In some embodiments, the compound is a compound of Formula (IA-c), (IB-c) or (IC-c), wherein each of R² and R³ is independently C₁-C₄ alkyl or C₅-C₆ cycloalkyl. In some embodiments, the compound is a compound of Formula (IA-c), (IB-c) or (IC-c), wherein each of R² and R³ is independently methyl or C₅-C₆ cycloalkyl. In some embodiments, the compound is a compound of Formula (IA-c), (IB-c) or (IC-c), wherein R² is methyl and R³ is C₅-C₆ cycloalkyl. In some embodiments, the compound is a compound of Formula (IA-c), (IB-c) or (IC-c), wherein R² is methyl and R³ is cyclopentyl. In some embodiments, the compound is a compound of Formula (IA-c), (IB-c) or (IC-c), wherein R² is methyl and R³ is cyclohexyl.

In some embodiments, the compound is a compound of Formula (IA-c), (IB-c) or (IC-c), wherein each of R² and R³ is independently C₁-C₄ alkyl or C₁-C₄ alkyl substituted with C₅-C₆ aryl. In some embodiments, the compound is a compound of Formula (IA-c), (IB-c) or (IC-c), wherein R² is benzyl and R³ is C₁-C₄ alkyl. In some embodiments, the compound is a compound of Formula (IA-c), (IB-c) or (IC-c), wherein R² is benzyl and R³ is methyl. In some embodiments, the compound is a compound of Formula (IA-c), (IB-c) or (IC-c), wherein R³ is benzyl and R² is C₁-C₄ alkyl. In some embodiments, the compound is a compound of Formula (IA-c), (IB-c) or (IC-c), wherein R³ is benzyl and R² is methyl.

In some embodiments, the compound is a compound of Formula (IA-c), (IB-c) or (IC-c), wherein each of R² and R³ is independently C₁-C₄ alkyl or C₁-C₄ alkyl substituted with C₂-C₃ alkenyl. In some embodiments, the compound is a compound of Formula (IA-c), (IB-c) or (IC-c), wherein R² is C₁-C₄ alkyl and R³ is C₁-C₄ alkyl substituted with C₂-C₃ alkenyl. In some embodiments, the compound is a compound of Formula (IA-c), (IB-c) or (IC-c), wherein R² is methyl and R³ is C₁-C₄ alkyl substituted with C₂ alkenyl. In some embodiments, the compound is a compound of Formula (IA-c), (IB-c) or (IC-c), wherein R³ is methyl and R² is C₁-C₄ alkyl substituted with C₂ alkenyl. In some embodiments, the compound is a compound of Formula (IA-c), (IB-c) or (IC-c), wherein R² is methyl and R³ is —CH₂CH═CH₂. In some embodiments, the compound is a compound of Formula (IA-c), (IB-c) or (IC-c), wherein R³ is methyl and R² is —CH₂CH═CH₂.

In some embodiments, the compound is a compound of Formula (IA-c), (IB-c) or (IC-c), wherein each of R² and R³ is independently C₁-C₄ alkyl or C₁-C₄ alkyl substituted with C₂-C₃ alkynyl. In some embodiments, the compound is a compound of Formula (IA-c), (IB-c) or (IC-c), wherein R² is C₁-C₄ alkyl and R³ is C₁-C₄ alkyl substituted with C₂-C₃ alkynyl. In some embodiments, the compound is a compound of Formula (IA-c), (IB-c) or (IC-c), wherein R² is methyl and R³ is C₁-C₄ alkyl substituted with C₂ alkynyl. In some embodiments, the compound is a compound of Formula (IA-c), (IB-c) or (IC-c), wherein R³ is methyl and R² is C₁-C₄ alkyl substituted with C₂ alkynyl. In some embodiments, the compound is a compound of Formula (IA-c), (IB-c) or (IC-c), wherein R² is methyl and R³ is —CH₂C≡CH. In some embodiments, the compound is a compound of Formula (IA-c), (IB-c) or (IC-c), wherein R³ is methyl and R² is —CH₂CH═CH.

In some embodiments, the compound is a compound of Formula (IA-c), (IB-c) or (IC-c), wherein R² and R³ together with the atoms to which they are attached combine to form 5-6 membered heterocyclyl, wherein each hydrogen atom in heterocyclyl is optionally substituted by halo or OR^{a}; and R^{a} is C₁-C₄ alkyl. In some embodiments, the compound is a compound of Formula (IA-c), (IB-c) or (IC-c), wherein R² and R³ together form a dioxolane optionally substituted with fluoro, methyl, or methyl substituted with fluoro. In some embodiments, the compound is a compound of Formula (IA-c), (IB-c) or (IC-c), wherein R² and R³ together form a dioxolane optionally substituted with fluoro. In some embodiments, the compound is a compound of Formula (IA-c), (IB-c) or (IC-c), wherein R² and R³ together with the oxygen atoms to which each is attached form —O—CF₂—O—, —O—CH₂—O— or —O—CHF—O—. In some embodiments, the compound is a compound of Formula (IA-c), (IB-c) or (IC-c), wherein R² and R³ together with the oxygen atoms to which each is attached form —O—CF₂—O—.

In some embodiments, the compound is a compound of Formula (IA-c), (IB-c) or (IC-c), wherein R⁴ is C₁-C₄ alkyl optionally substituted with deuterium, halo, C₃-C₆ cycloalkyl, C₆ aryl or 5-6 membered heteroaryl; and the cycloalkyl, aryl or heteroaryl is optionally substituted with —OR^{a} or R^{a} and R^{a} is C₁-C₄ alkyl. In some embodiments, the compound is a compound of Formula (IA-c), (IB-c) or (IC-c), wherein R⁴ is C₁-C₄ alkyl. In some embodiments, the compound is a compound of Formula (IA-c), (IB-c) or (IC-c), wherein R⁴ is methyl. In some embodiments, the compound is a compound of Formula (IA-c), (IB-c) or (IC-c), wherein R⁴ is ethyl. In some embodiments, the compound is a compound of Formula (IA-c), (IB-c) or (IC-c), wherein R⁴ is propyl. In some embodiments, the compound is a compound of Formula (IA-c), (IB-c) or (IC-c), wherein R⁴ is isopropyl. In some embodiments, the compound is a compound of Formula (IA-c), (IB-c) or (IC-c), wherein R⁴ is C₁-C₄ alkyl substituted with C₃-C₆ cycloalkyl. In some embodiments, the compound is a compound of Formula (IA-c), (IB-c) or (IC-c), wherein R⁴ is methyl substituted with C₃-C₆ cycloalkyl. In some embodiments, the compound is a compound of Formula (IA-c), (IB-c) or (IC-c), wherein R⁴ is methyl substituted with cyclopropyl. In some embodiments, the compound is a compound of Formula (IA-c), (IB-c) or (IC-c), wherein R⁴ is methyl substituted with cyclobutyl. In some embodiments, the compound is a compound of Formula (IA-c), (IB-c) or (IC-c), wherein R⁴ is methyl substituted with cyclohexyl. In some embodiments, the compound is a compound of Formula (IA-c), (IB-c) or (IC-c), wherein R⁴ is benzyl optionally substituted with methyl or methoxy. In some embodiments, the compound is a compound of Formula (IA-c), (IB-c) or (IC-c), wherein R⁴ is benzyl optionally substituted with methoxy. In some embodiments, the compound is a compound of Formula (IA-c), (IB-c) or (IC-c), wherein R⁴ is C₁-C₄ alkyl optionally substituted with deuterium. In some embodiments, the compound is a compound of Formula (IA-c), (IB-c) or (IC-c), wherein R⁴ is methyl substituted with deuterium. In some embodiments, the compound is a compound of Formula (IA-c), (IB-c) or (IC-c), wherein R⁴ is —CD₃. In some embodiments, the compound is a compound of Formula (IA-c), (IB-c) or (IC-c), wherein R⁴ is —CF₃.

In some embodiments, the compound is a compound of Formula (IA-c). In some embodiments, the compound is a compound of Formula (IB-b). In some embodiments, the compound is a compound of Formula (IC-c).

In some embodiments, the compound is a compound of Formula (IA-d), (IB-d), or (IC-d):

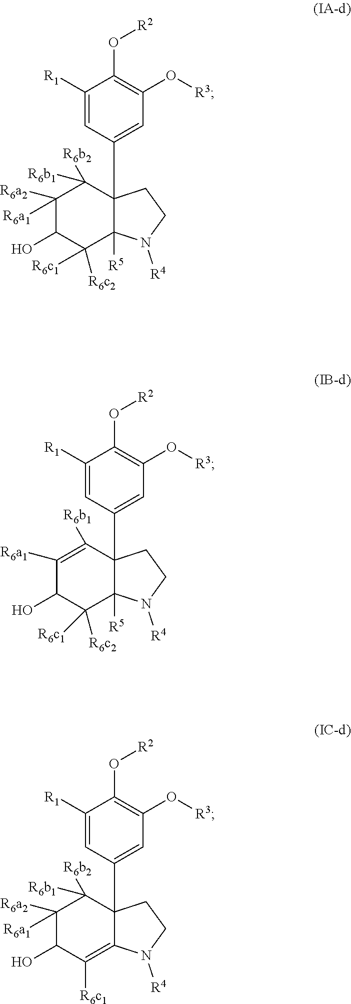

or a pharmaceutically acceptable salt thereof; wherein
each R₆ₐ₁, R₆ₐ₂, R_{6b1}, R_{6b2}, R_{6c1} and R_{6c2} is independently hydrogen, deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or R₆ₐ₂ and R_{6b2} combine to form a cycloalkyl, or heterocyclyl, wherein each hydrogen atom in cycloalkyl, and heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or R_{6b2} and R_{6c2} combine to form a cycloalkyl, or heterocyclyl, wherein each hydrogen atom in cycloalkyl, or heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or R_{6c1} and R_{6c2} combine to form a cycloalkyl, or heterocyclyl, wherein each hydrogen atom in cycloalkyl, or heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}.

In some embodiments, the compound is a compound of Formula (IA-d), (IB-d), or (IC-d), wherein each of R₆ₐ₁, R₆ₐ₂, R_{6b1}, R_{6b2}, R_{6c1} and R_{6c2} is hydrogen.

In some embodiments, the compound is a compound of Formula (IA-d), (IB-d), or (IC-d), wherein each of R₆ₐ₁, R₆ₐ₂, R_{6b1}, R_{6b2}, R_{6c1} and R_{6c2} is hydrogen.

In some embodiments, the compound is a compound of Formula (IA-d), (IB-d), or (IC-d), wherein each of R₆ₐ₁, R₆ₐ₂, R_{6b1}, R_{6b2}, R_{6c1} and R_{6c2} is independently hydrogen or C₁-C₄ alkyl. In some embodiments, the compound is a compound of Formula (IA-d), (IB-d), or (IC-d), wherein each of R₆ₐ₁, R₆ₐ₂, R_{6b1}, R_{6b2}, R_{6c1} and R_{6c2} is independently hydrogen or C₁-C₄ alkyl, provided that only one of R₆ₐ₁, R₆ₐ₂, R_{6b1}, R_{6b2}, R_{6c1} and R_{6c2} is C₁-C₄ alkyl.

In some embodiments, the compound is a compound of Formula (IA-d), (IB-d), or (IC-d), wherein R₆ₐ₁ is C₁-C₄ alkyl and each of R₆ₐ₂, R_{6b1}, R_{6b2}, R_{6c1} and R_{6c2} is independently hydrogen. In some embodiments, the compound is a compound of Formula (IA-d), (IB-d), or (IC-d), wherein R₆ₐ₁ is methyl and each of R₆ₐ₂, R_{6b1}, R_{6b2}, R_{6c1} and R_{6c2} is independently hydrogen. In some embodiments, the compound is a compound of Formula (IA-d), (IB-d), or (IC-d), wherein R₆ₐ₁ is ethyl and each of R₆ₐ₂, R_{6b1}, R_{6b2}, R_{6c1} and R_{6c2} is independently hydrogen. In some embodiments, the compound is a compound of Formula (IA-d), (IB-d), or (IC-d), wherein R₆ₐ₁ is propyl and each of R₆ₐ₂, R_{6b1}, R_{6b2}, R_{6c1} and R_{6c2} is independently hydrogen. In some embodiments, the compound is a compound of Formula (IA-d), (IB-d), or (IC-d), wherein R₆ₐ₁ is butyl and each of R₆ₐ₂, R_{6b1}, R_{6b2}, R_{6c1} and R_{6c2} is independently hydrogen.

In some embodiments, the compound is a compound of Formula (IA-d), (IB-d), or (IC-d), wherein R_{6b1} is C₁-C₄ alkyl and each of R₆ₐ₁, R₆ₐ₂, R_{6b2}, R_{6c1} and R_{6c2} is independently hydrogen. In some embodiments, the compound is a compound of Formula (IA-d), (IB-d), or (IC-d), wherein R_{6b1} is methyl and each of R₆ₐ₂, R₆ₐ₁, R_{6b2}, R_{6c1} and R_{6c2} is independently hydrogen. In some embodiments, the compound is a compound of Formula (IA-d), (IB-d), or (IC-d), wherein R_{6b1} is ethyl and each of R₆ₐ₂, R₆ₐ₁, R_{6b2}, R_{6c1} and R_{6c2} is independently hydrogen. In some embodiments, the compound is a compound of Formula (IA-d), (IB-d), or (IC-d), wherein R_{6b1} is propyl and each of R₆ₐ₂, R₆ₐ₁, R_{6b2}, R_{6c1} and R_{6c2} is independently hydrogen. In some embodiments, the compound is a compound of Formula (IA-d), (IB-d), or (IC-d), wherein R_{6b1} is butyl and each of R₆ₐ₂, R₆ₐ₁, R_{6b2}, R_{6c1} and R_{6c2} is independently hydrogen.

In some embodiments, the compound is a compound of Formula (IA-d), (IB-d), or (IC-d), wherein R_{6c1} is C₁-C₄ alkyl and each of R₆ₐ₁, R₆ₐ₂, R_{6b2}, R_{6b1} and R_{6c2} is independently hydrogen. In some embodiments, the compound is a compound of Formula (IA-d), (IB-d), or (IC-d), wherein R_{6c1} is methyl and each of R₆ₐ₂, R₆ₐ₁, R_{6b2}, R_{6b1} and R_{6c2} is independently hydrogen. In some embodiments, the compound is a compound of Formula (IA-d), (IB-d), or (IC-d), wherein R_{6c1} is ethyl and each of R₆ₐ₂, R₆ₐ₁, R_{6b2}, R_{6b1} and R_{6c2} is independently hydrogen. In some embodiments, the compound is a compound of Formula (IA-d), (IB-d), or (IC-d), wherein R_{6c1} is propyl and each of R₆ₐ₂, R₆ₐ₁, R_{6b2}, R_{6b1} and R_{6c2} is independently hydrogen. In some embodiments, the compound is a compound of Formula (IA-d), (IB-d), or (IC-d), wherein R_{6c1} is butyl and each of R₆ₐ₂, R₆ₐ₁, R_{6b2}, R_{6b1} and R_{6c2} is independently hydrogen. In some embodiments, the compound is a compound of Formula (IA-d), (IB-d), or (IC-d), wherein each of R_{6c1} and R_{6c2} is methyl and each of R₆ₐ₁, R₆ₐ₂, R_{6b2}, and R_{6b1} is independently hydrogen.

In some embodiments, the compound is a compound of Formula (IA-d), (IB-d), or (IC-d), wherein R₆ₐ₁ is —CF₃ and each of R₆ₐ₂, R_{6b1}, R_{6b2}, R_{6c1} and R_{6c2} is independently hydrogen. In some embodiments, the compound is a compound of Formula (IA-d), (IB-d), or (IC-d), wherein R_{6b1} is —CF₃ and each of R₆ₐ₂, R₆ₐ₁, R_{6b2}, R_{6c1} and R_{6c2} is independently hydrogen. In some embodiments, the compound is a compound of Formula (IA-d), (IB-d), or (IC-d), wherein R_{6c1} is —CF₃ and each of R₆ₐ₂, R_{6b1}, R_{6b2}, R_{6b1} and R_{6c2} is independently hydrogen.

In some embodiments, the compound is a compound of Formula (IA-d), (IB-d), or (IC-d), wherein R₆ₐ₁ is —Si(CH₃)₃ and each of R₆ₐ₂, R_{6b1}, R_{6b2}, R_{6c1} and R_{6c2} is independently hydrogen. In some embodiments, the compound is a compound of Formula (IA-d), (IB-d), or (IC-d), wherein R_{6b1} is —Si(CH₃)₃ and each of R₆ₐ₂, R₆ₐ₁, R_{6b2}, R_{6c1} and R_{6c2} is independently hydrogen. In some embodiments, the compound is a compound of Formula (IA-d), (IB-d), or (IC-d), wherein R_{6c1} is —Si(CH₃)₃ and each of R₆ₐ₂, R_{6b1}, R_{6b2}, R_{6b1} and R_{6c2} is independently hydrogen.

In some embodiments, the compound is a compound of Formula (IA-d), (IB-d), or (IC-d), wherein R₆ₐ₁ is acetyl and each of R₆ₐ₂, R_{6b1}, R_{6b2}, R_{6c1} and R_{6c2} is independently hydrogen. In some embodiments, the compound is a compound of Formula (IA-d), (IB-d), or (IC-d), wherein R_{6b1} is acetyl and each of R₆ₐ₂, R₆ₐ₁, R_{6b2}, R_{6c1} and R_{6c2} is independently hydrogen. In some embodiments, the compound is a compound of Formula (IA-d), (IB-d), or (IC-d), wherein R_{6c1} is acetyl and each of R₆ₐ₂, R_{6b1}, R_{6b2}, R_{6b1} and R_{6c2} is independently hydrogen.

In some embodiments, the compound is a compound of Formula (IA-d), or (IB-d), wherein R_{6c1} and R_{6c2} combine to form a C₃-C₆ cycloalkyl, wherein each hydrogen atom in the cycloalkyl is optionally substituted by halo, hydroxy, alkyl, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}. In some embodiments, the compound is a compound of Formula (IA-d), or (IB-d), wherein R_{6c1} and R_{6c2} combine to form a cyclopropyl. In some embodiments, the compound is a compound of Formula (IA-d), or (IB-d), wherein R_{6c1} and R_{6c2} combine to form an unsubstituted cyclopropyl. In some embodiments, the compound is a compound of Formula (IA-d), or (IC-d), wherein R_{6b1} and R_{6b2} combine to form an unsubstituted cyclopropyl, and each of R₆ₐ₁, R₆ₐ₂, R_{6c1} and R_{6c2} are each hydrogen.

In some embodiments, the compound is a compound of Formula (IA-d), (IB-d), or (IC-d), wherein each of R₆ₐ₁, R₆ₐ₂, R_{6b1}, R_{6b2}, R_{6c1} and R_{6c2} is independently hydrogen or deuterium. In some embodiments, the compound is a compound of Formula (IA-d), (IB-d), or (IC-d), wherein R_{6c1} is deuterium and each of R₆ₐ₁, R₆ₐ₂, R_{6b1}, R_{6b2}, and R_{6c2} is independently hydrogen. In some embodiments, the compound is a compound of Formula (IA-d), or (IB-d), wherein R_{6c1} and R_{6c2} are each deuterium and each of R₆ₐ₁, R₆ₐ₂, R_{6b1}, and R_{6b2} is independently hydrogen. In some embodiments, the compound is a compound of Formula (IA-d), (IB-d), or (IC-d), wherein R₆ₐ₁ is deuterium and each of R₆ₐ₂, R_{6b1}, R_{6b2}, R_{6c1}, and R_{6c2} is independently hydrogen. In some embodiments, the compound is a compound of Formula (IA-d), or (IC-d), wherein each of R₆ₐ₁ and R₆ₐ₁ is deuterium and each of R₆ₐ₂, R_{6b1}, R_{6b2}, R_{6c1}, and R_{6c2} is independently hydrogen. In some embodiments, the compound is a compound of Formula (IA-d), or (IC-d), wherein each of R₆ₐ₁, R₆ₐ₁, R_{6c1}, and R_{6c2} is deuterium and each of R_{6b1} and R_{6b2}, is independently hydrogen.

In some embodiments, the compound is a compound of Formula (IA-d), or (IC-d), wherein each of R₆ₐ₁, and R_{6b1} together form a fused heterocyclyl. In some embodiments, the compound is a compound of Formula (IA-d), or (IC-d), wherein each of R₆ₐ₁, and R_{6b1} together form an epoxide. In some embodiments, the compound is a compound of Formula (IA-d), or (IC-d), wherein each of R₆ₐ₁, and R_{6b1} together form an epoxide, and each of R₆ₐ₂, R_{6b2}, R_{6c1} and R_{6b2} is independently hydrogen.

In some embodiments, the compound is a compound of Formula (IA-d), (IB-d), or (IC-d), wherein each of R_{6b1}, and R_{6c1} together form a bridging cycloalkyl. In some embodiments, the compound is a compound of Formula (IA-d), (IB-d), or (IC-d), wherein each of R_{6b1}, and R_{6c1} are together a C₁-C₄ alkyl forming a bridging cycloalkyl. In some embodiments, the compound is a compound of Formula (IA-d), (IB-d), or (IC-d), wherein each of R_{6b1}, and R_{6c1} are together a C₁-C₄ alkyl forming a bridging cycloalkyl, and R₆ₐ₁, R₆ₐ₂, R_{6b2}, and R_{6c2} are each hydrogen. In some embodiments, the compound is a compound of Formula (IA-d), (IB-d), or (IC-d), wherein each of R_{6b1}, and R_{6c1} are together a C₁ alkyl forming a bridging cycloalkyl. In some embodiments, the compound is a compound of Formula (IA-d), (IB-d), or (IC-d), wherein each of R_{6b1}, and R_{6c1} are together a C₂ alkyl forming a bridging cycloalkyl, and R₆ₐ₁, R₆ₐ₂, R_{6b2}, and R_{6c2} are each hydrogen. In some embodiments, the compound is a compound of Formula (IA-d), (IB-d), or (IC-d), wherein each of R_{6b1}, and R_{6c1} are together a C₃ alkyl forming a bridging cycloalkyl, and R₆ₐ₁, R₆ₐ₂, R_{6b2}, and R_{6c2} are each hydrogen. In some embodiments, the compound is a compound of Formula (IA-d), (IB-d), or (IC-d), wherein each of R_{6b1}, and R_{6c1} are together a C₄ alkyl forming a bridging cycloalkyl, and R₆ₐ₁, R₆ₐ₂, R_{6b2}, and R_{6c2} are each hydrogen. In some embodiments, the compound is a compound of Formula (IA-d), wherein each of R_{6b1}, and R_{6c1} are together a C₁ alkyl forming a bridging cycloalkyl, and R₆ₐ₁, R₆ₐ₂, R_{6b2}, and R_{6c2} are each hydrogen. In some embodiments, the compound is a compound of Formula (IA-d), wherein each of R_{6b1}, and R_{6c1} are together a C₂ alkyl forming a bridging cycloalkyl, and R₆ₐ₁, R₆ₐ₂, R_{6b2}, and R_{6c2} are each hydrogen. In some embodiments, the compound is a compound of Formula (IA-d), wherein each of R_{6b1}, and R_{6c1} are together a C₃ alkyl forming a bridging cycloalkyl, and R₆ₐ₁, R₆ₐ₂, R_{6b2}, and R_{6c2} are each hydrogen.

In some embodiments, the compound is a compound of Formula (IA-d), (IB-d), or (IC-d), wherein R_{6b1} is phenyl and each of R₆ₐ₁, R₆ₐ₂, R_{6b2}, R_{6c1} and R_{6c2} is independently hydrogen. In some embodiments, the compound is a compound of Formula (IA-d), wherein R_{6b1} is phenyl and each of R₆ₐ₁, R₆ₐ₂, R_{6b2}, R_{6c1} and R_{6c2} is independently hydrogen.

In some embodiments, the compound is a compound of Formula (IA-c):

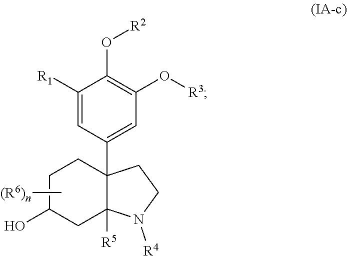

or a pharmaceutically acceptable salt thereof; wherein
R¹ is deuterium, C₁-C₄ alkyl optionally substituted with deuterium or halo, cyclopropyl, acetyl, amido, cyano, or C₂-C₃ alkenyl;
each of R² and R³ is independently C₁-C₄ alkyl optionally substituted with halo, deuterium or cyclopropyl, C₂-C₃ alkenyl, C₂-C₃ alkynyl, C₃-C₆ cycloalkyl, OR^{a} or benzyl;
R⁴ is methyl;
R⁵ is hydrogen or deuterium;
each R⁶ is independently deuterium, C₁-C₄ alkyl optionally substituted with halo or deuterium, —Si(alkyl)₃, or acetyl;
or two R⁶s on adjacent carbon atoms together with the carbon atoms to which they are attached combine to form an epoxide;
or two R⁶s on different carbon atoms together with the carbon atom to which they are attached combine to form a bridging C₅-C₈ cycloalkyl;
or two R⁶s on a single carbon atom together with the carbon atom to which they are attached combine to form a C₃-C₈ cycloalkyl;
n is 0, 1, 2, 3, 4, 5, or 6;
R^{a} is H, or C₁-C₄ alkyl,
provided the compound is not:

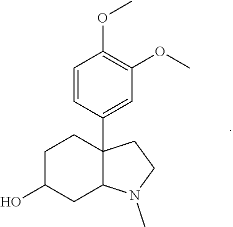 [SMILES: COc1ccc(C23CCC(O)CC2N(C)CC3)cc1OC]

In some embodiments, the compound is a compound of Formula (IA-c):

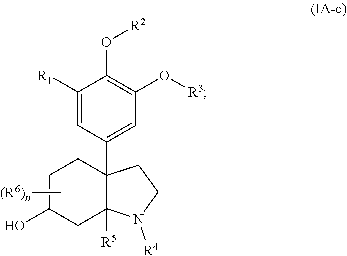

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen or deuterium;
each R⁶ is independently deuterium, C₁-C₄ alkyl optionally substituted with halo or deuterium, —Si(CH₃)₃, or acetyl,
or two R⁶s on adjacent carbon atoms together with the carbon atoms to which they are attached combine to form an epoxide;
or two R⁶s on different carbon atoms together with the carbon atom to which they are attached combine to form a bridging C₅-C₇ cycloalkyl;
or two R⁶s on a single carbon atom together with the carbon atom to which they are attached combine to form a cyclopropyl;
n is 0, 1, 2, 3, 4, 5, or 6;
provided the compound is not:

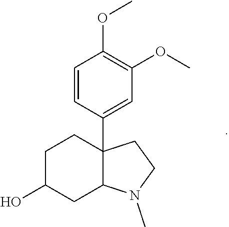 [SMILES: COc1ccc(C23CCC(O)CC2N(C)CC3)cc1OC]

In some embodiments, the compound is a compound of Formula (IA-c):

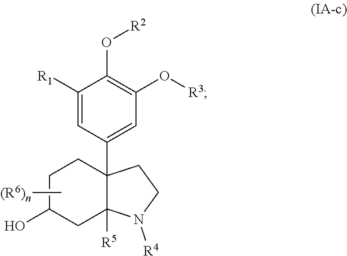

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen;
each R⁶ is independently deuterium, C₁-C₄ alkyl optionally substituted with halo or deuterium, —Si(CH₃)₃, or acetyl,
or two R⁶s on adjacent carbon atoms together with the carbon atoms to which they are attached combine to form an epoxide;
or two R⁶s on different carbon atoms together with the carbon atom to which they are attached combine to form a bridging C₅-C₇ cycloalkyl;
or two R⁶s on a single carbon atom together with the carbon atom to which they are attached combine to form a cyclopropyl;
n is 0, 1 or 2;
provided the compound is not:

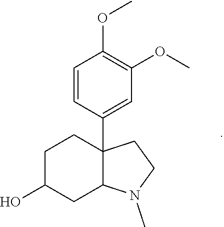 [SMILES: COc1ccc(C23CCC(O)CC2N(C)CC3)cc1OC]

In some embodiments, the compound is a compound of Formula (IA-c):

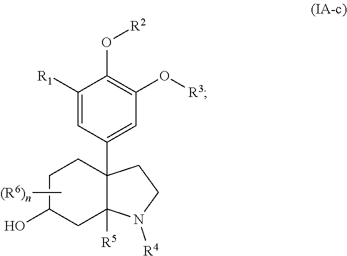

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen;
R⁶ is methyl;
n is 1.

In some embodiments, the compound is a compound of Formula (IA-c):

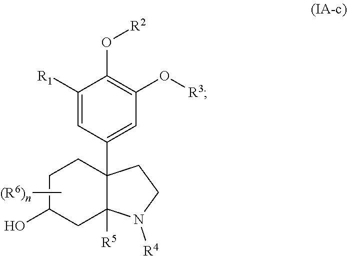

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen; and
n is 0;
provided the compound is not:

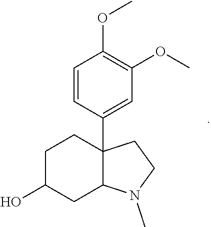 [SMILES: COc1ccc(C23CCC(O)CC2N(C)CC3)cc1OC]

In some embodiments, the compound is a compound of Formula (IB-c):

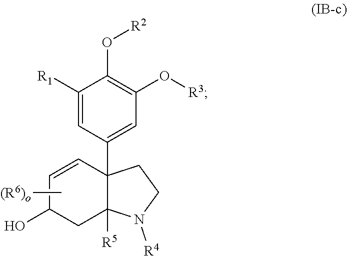

or a pharmaceutically acceptable salt thereof; wherein
R¹ is deuterium, C₁-C₄ alkyl optionally substituted with deuterium or halo, cyclopropyl, acetyl, amido, cyano, or C₂-C₃ alkenyl;
each of R² and R³ is independently C₁-C₄ alkyl optionally substituted with halo, deuterium or cyclopropyl, C₂-C₃ alkenyl, C₂-C₃ alkynyl, C₃-C₆ cycloalkyl, OR^{a} or benzyl;
R⁴ is methyl;
R⁵ is hydrogen or deuterium;
each R⁶ is independently deuterium, C₁-C₄ alkyl optionally substituted with halo or deuterium, —Si(alkyl)₃, or acetyl;
or two R⁶s on adjacent carbon atoms together with the carbon atoms to which they are attached combine to form an epoxide;
or two R⁶s on different carbon atoms together with the carbon atom to which they are attached combine to form a bridging C₅-C₈ cycloalkyl;
or two R⁶s on a single carbon atom together with the carbon atom to which they are attached combine to form a C₃-C₈ cycloalkyl;
o is 0, 1, 2, 3, or 4;
R^{a} is H, or C₁-C₄ alkyl,
provided the compound is not:

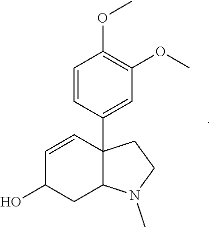 [SMILES: COc1ccc(C23C=CC(O)CC2N(C)CC3)cc1OC]

In some embodiments, the compound is a compound of Formula (IB-c):

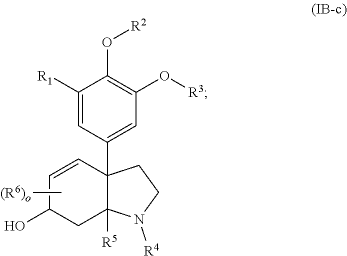

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen or deuterium;
each R⁶ is independently deuterium, C₁-C₄ alkyl optionally substituted with halo or deuterium, —Si(CH₃)₃, or acetyl,
or two R⁶s on adjacent carbon atoms together with the carbon atoms to which they are attached combine to form an epoxide;
or two R⁶s on different carbon atoms together with the carbon atom to which they are attached combine to form a bridging C₅-C₇ cycloalkyl;
or two R⁶s on a single carbon atom together with the carbon atom to which they are attached combine to form a cyclopropyl;
o is 0, 1, 2, 3, or 4;
provided the compound is not:

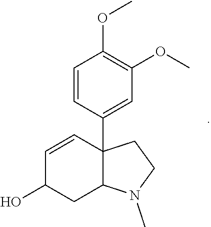 [SMILES: COc1ccc(C23C=CC(O)CC2N(C)CC3)cc1OC]

In some embodiments the compound is a compound of Formula (IB-c):

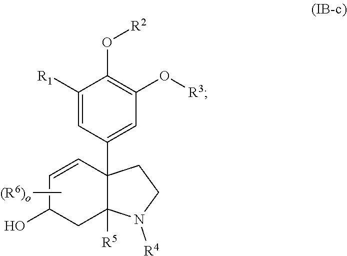

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen;
each R⁶ is independently deuterium, C₁-C₄ alkyl optionally substituted with halo or deuterium, —Si(CH₃)₃, or acetyl,
or two R⁶s on adjacent carbon atoms together with the carbon atoms to which they are attached combine to form an epoxide;
or two R⁶s on different carbon atoms together with the carbon atom to which they are attached combine to form a bridging C₅-C₇ cycloalkyl;
or two R⁶s on a single carbon atom together with the carbon atom to which they are attached combine to form a cyclopropyl;
o is 0, 1 or 2;
provided the compound is not:

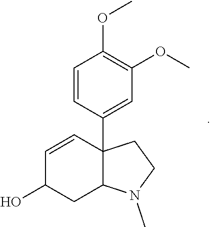 [SMILES: COc1ccc(C23C=CC(O)CC2N(C)CC3)cc1OC]

In some embodiments, the compound is a compound of Formula (IB-c):

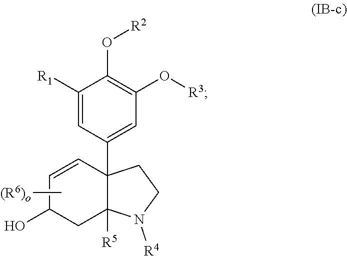

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen;
R⁶ is methyl;
n is 1.

In some embodiments, the compound is a compound of Formula (IB-c):

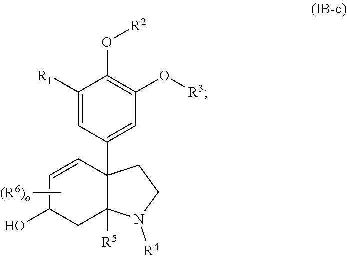

or a pharmaceutically acceptable salt thereof, wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen; and
n is 0;
provided the compound is not:

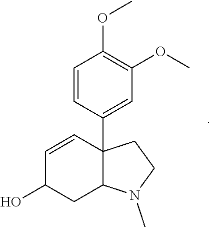 [SMILES: COc1ccc(C23C=CC(O)CC2N(C)CC3)cc1OC]

In some embodiments, the compound is a compound of Formula (IC-c):

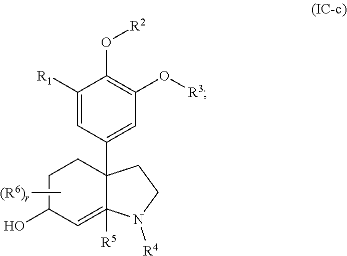

or a pharmaceutically acceptable salt thereof; wherein
R¹ is deuterium, C₁-C₄ alkyl optionally substituted with deuterium or halo, cyclopropyl, acetyl, amido, cyano, or C₂-C₃ alkenyl;
each of R² and R³ is independently C₁-C₄ alkyl optionally substituted with halo, deuterium, cyclopropyl, C₂-C₃ alkenyl, C₂-C₃ alkynyl, C₃-C₆ cycloalkyl, OR^{a} or benzyl;
R⁴ is methyl;
R⁵ is hydrogen or deuterium;
each R⁶ is independently deuterium, C₁-C₄ alkyl optionally substituted with halo or deuterium, —Si(alkyl)₃, or acetyl;
or two R⁶s on adjacent carbon atoms together with the carbon atoms to which they are attached combine to form an epoxide;
or two R⁶s on different carbon atoms together with the carbon atom to which they are attached combine to form a bridging C₅-C₈ cycloalkyl;
or two R⁶s on a single carbon atom together with the carbon atom to which they are attached combine to form a C₃-C₈ cycloalkyl;
r is 0, 1, 2, 3, 4 or 5;
R^{a} is H, or C₁-C₄ alkyl,
provided the compound is not:

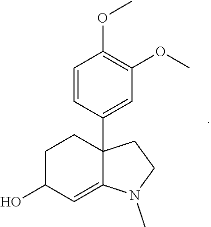 [SMILES: COc1ccc(C23CCC(O)C=C2N(C)CC3)cc1OC]

In some embodiments, the compound is a compound of Formula (IC-c):

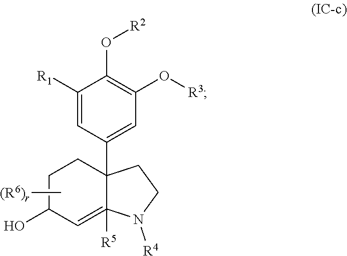

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen or deuterium;
each R⁶ is independently deuterium, C₁-C₄ alkyl optionally substituted with halo or deuterium, —Si(CH₃)₃, or acetyl,
or two R⁶s on adjacent carbon atoms together with the carbon atoms to which they are attached combine to form an epoxide;
or two R⁶s on different carbon atoms together with the carbon atom to which they are attached combine to form a bridging C₅-C₇ cycloalkyl;
or two R⁶s on a single carbon atom together with the carbon atom to which they are attached combine to form a cyclopropyl;
r is 0, 1, 2, 3, 4, or 5;
provided the compound is not:

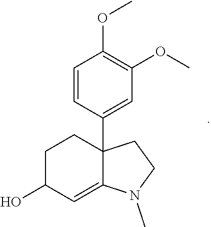 [SMILES: COc1ccc(C23CCC(O)C=C2N(C)CC3)cc1OC]

In some embodiments, the compound is a compound of Formula (IC-c):

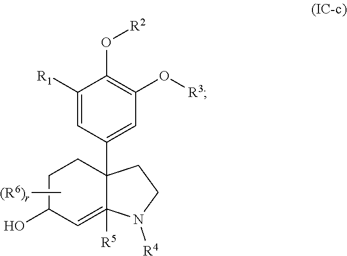

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen;
each R⁶ is independently deuterium, C₁-C₄ alkyl optionally substituted with halo or deuterium, —Si(CH₃)₃, or acetyl,
or two R⁶s on adjacent carbon atoms together with the carbon atoms to which they are attached combine to form an epoxide;
or two R⁶s on different carbon atoms together with the carbon atom to which they are attached combine to form a bridging C₅-C₇ cycloalkyl;
or two R⁶s on a single carbon atom together with the carbon atom to which they are attached combine to form a cyclopropyl;
r is 0, 1 or 2;
provided the compound is not:

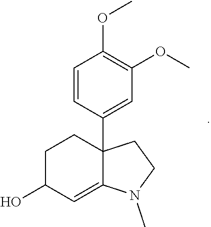 [SMILES: COc1ccc(C23CCC(O)C=C2N(C)CC3)cc1OC]

In some embodiments, the compound is a compound of Formula (IB-c):

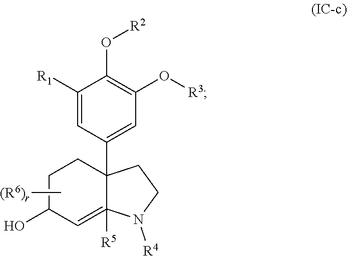

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen;
R⁶ is methyl;
r is 1.

In some embodiments, the compound is a compound of Formula (IC-c):

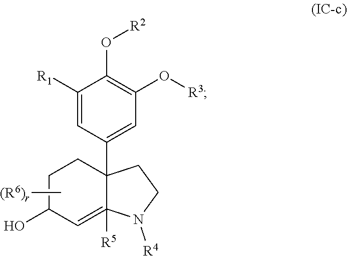

or a pharmaceutically acceptable salt thereof; wherein
R¹ is C₁-C₄ alkyl optionally substituted with deuterium or fluoro, cyclopropyl, acetyl, amido, cyano, or C₂-C₃ alkenyl;
each of R² and R³ is independently C₁-C₄ alkyl optionally substituted with fluoro, deuterium or cyclopropyl, C₂-C₃ alkenyl, C₂-C₃ alkynyl, C₃-C₆ cycloalkyl, methoxy or benzyl;
R⁴ is methyl;
R⁵ is hydrogen; and
r is 0;
provided the compound is not:

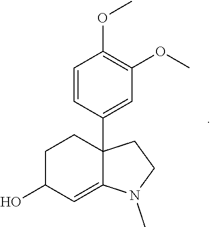 [SMILES: COc1ccc(C23CCC(O)C=C2N(C)CC3)cc1OC]

In certain aspects, the invention relates to compounds Formula (IIA) (IIB) or (IIC):

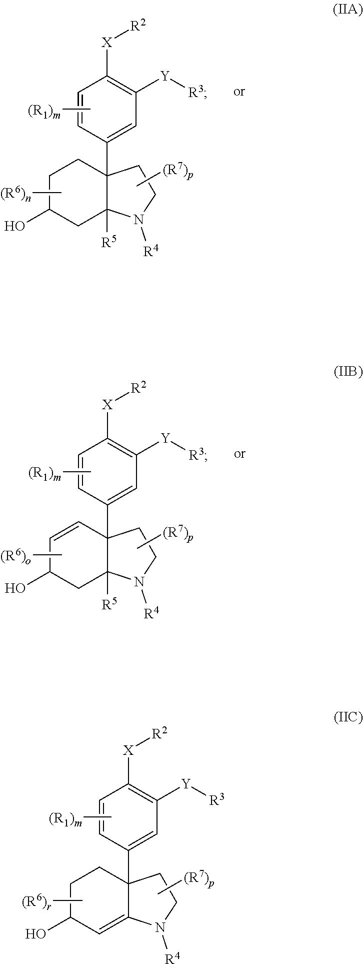

or a pharmaceutically acceptable salt thereof; wherein
X is O or S;
Y is O or S;
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R⁴ is H or alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a};
R⁵ is H, deuterium, or methyl;
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form cycloalkyl, heterocyclyl or heteroaryl, wherein each hydrogen atom in cycloalkyl, heterocyclyl, and 5 heteroaryl is optionally substituted by halo, hydroxy, alkoxy, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, alkyl, —OH, or —NH₂; wherein each hydrogen atom in alkyl is optionally substituted by —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl;
provided the compound is not:

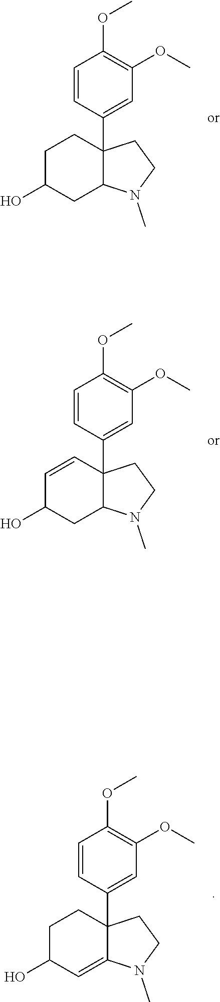 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

In certain embodiments, the compound is of Formula (IIA-a), (IIA-b), (IIB-a), (IIB-b), (IIC-a) or (IIC-b):

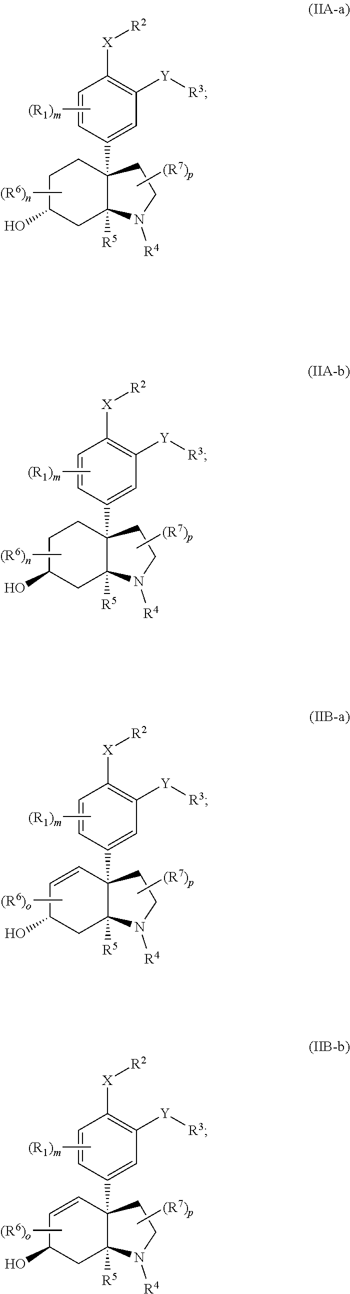

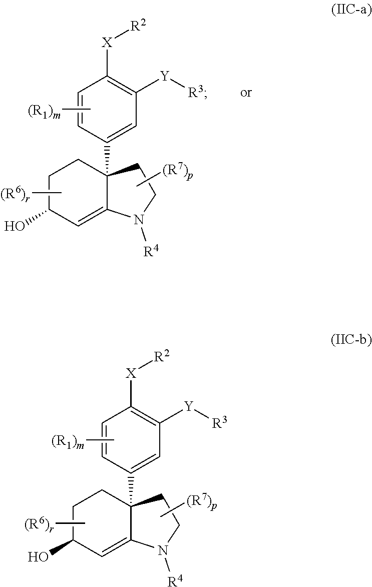

or a pharmaceutically acceptable salt thereof; wherein
X is O or S;
Y is O or S;
R¹, R₂, R₃, R₄, R₅, R₆, o, m and p are each as described with respect to Formula (IIA), (IIB) and (IIC); and
provided the compound is not:

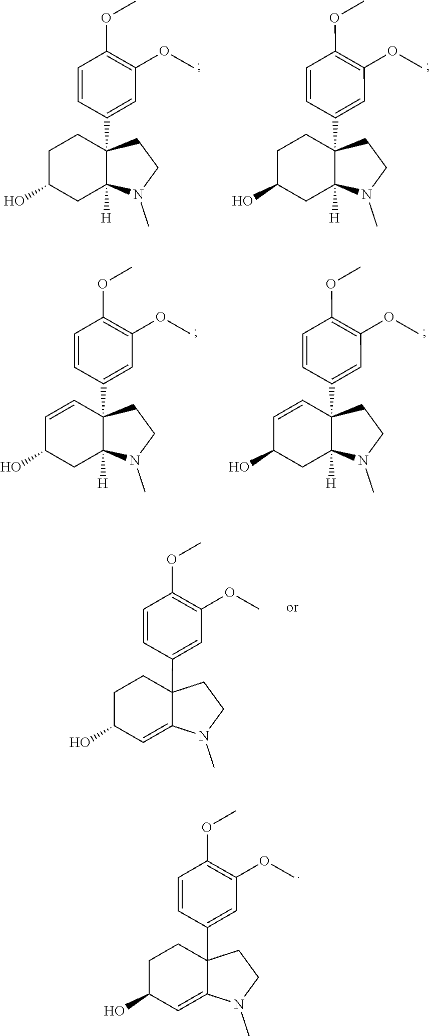

In certain embodiments, R⁴ in Formula (IIA), (IIB) or (IIC) or in Formula (IIA-a), (IIA-b), (IIB-a), (IIB-b), (IIC-a) or (IIC-b) is methyl. In certain embodiments, R⁵ in Formula (IIA), (IIB) or (IIC) or in Formula (IIA-a), (IIA-b), (IIB-a), (IIB-b), (IIC-a) or (IIC-b) is H. In certain embodiments, o in Formula (IIA), (IIB) or (IIC) or in Formula (IIA-a), (IIA-b), (IIB-a), (IIB-b), (IIC-a) or (IIC-b) is 0. In certain embodiments, p in Formula (IIA), (IIB) or (IIC) or in Formula (IIA-a), (IIA-b), (IIB-a), (IIB-b), (IIC-a) or (IIC-b) is 0. In certain embodiments, m in Formula (IIA), (IIB) or (IIC) or in Formula (IIA-a), (IIA-b), (IIB-a), (IIB-b), (IIC-a) or (IIC-b) is 0 or 1. In certain aspects, the invention relates to compounds Formula (IIIA), (IIIB) or (IIIC):

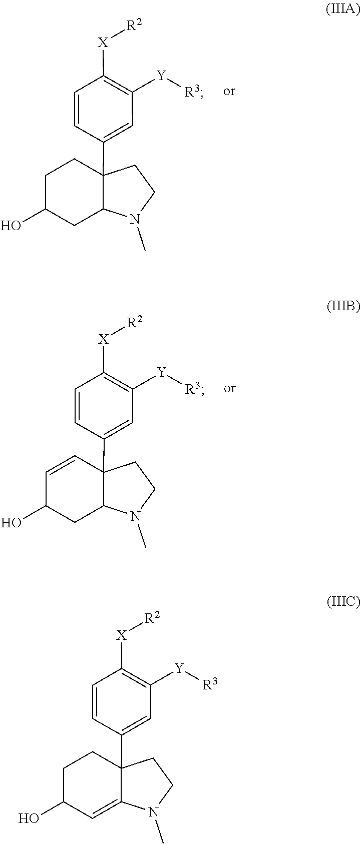

or a pharmaceutically acceptable salt thereof; wherein
X and Y are each independently O or S;
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
each R^{a} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl;
provided the compound is not:

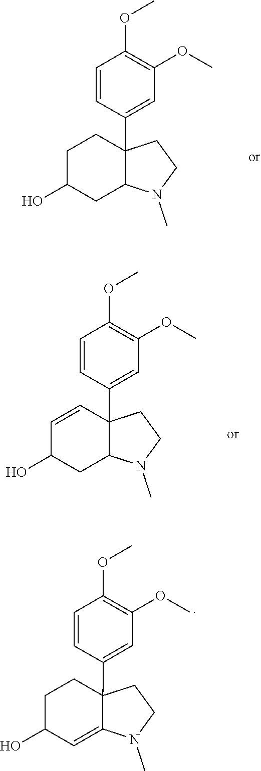 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

In certain aspects, the invention relates to compounds Formula (IIIA), (IIIB) or (IIIC):

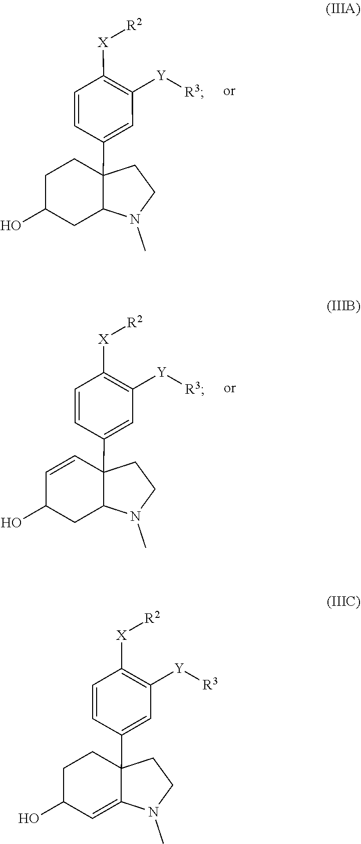

or a pharmaceutically acceptable salt thereof; wherein
each of X and Y are independently O or S;
each of R² and R³ is independently selected from the group consisting of: C₁₋₆ alkyl, C₂₋₆ alkenyl, C₂₋₆ alkynyl, 3-to-8-membered carbocycle, 3- to 8-membered heterocycle, hydrogen, deuterium or halogen; and
provided that if A and B are both O, then R² and R³ are not both methyl or are not both hydrogen.

In certain aspects, the invention relates to compounds Formula (IV):

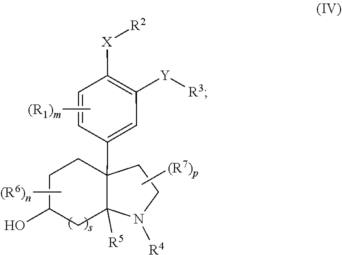

or a pharmaceutically acceptable salt thereof; wherein
X is O or S;
Y is O or S;
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R⁴ is H or alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a};
R⁵ is H, deuterium, or methyl;
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form cycloalkyl, heterocyclyl or heteroaryl, wherein each hydrogen atom in cycloalkyl, heterocyclyl, and 5 heteroaryl is optionally substituted by halo, hydroxy, alkoxy, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, alkyl, —OH, or —NH₂; wherein each hydrogen atom in alkyl is optionally substituted by —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
s is 1 or 2;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl;
provided the compound is not:

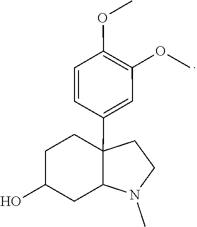 [SMILES: COc1ccc(C23CCC(O)CC2N(C)CC3)cc1OC]

In certain aspects, the invention relates to compounds Formula (IVA):

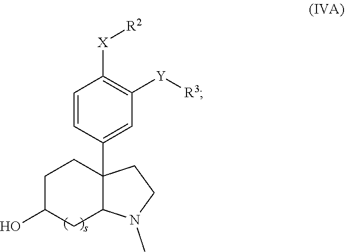

or a pharmaceutically acceptable salt thereof; wherein
each of X and Y are independently O or S;
each of R² and R³ is independently selected from the group consisting of: C₁₋₆ alkyl, C₂₋₆ alkenyl, C₂₋₆ alkynyl, 3-to-8-membered carbocycle, 3- to 8-membered heterocycle, hydrogen, deuterium or halogen;
s is 1 or 2; and
provided that if A and B are both O, then R² and R³ are not both methyl or are not both hydrogen.

In certain aspects, the invention relates to compounds Formula (IVA):

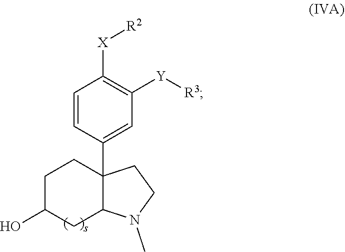

or a pharmaceutically acceptable salt thereof; wherein
each of X and Y are independently O or S;
each of R² and R³ is independently selected from the group consisting of: C₁₋₆ alkyl, C₂₋₆ alkenyl, C₂₋₆ alkynyl, 3-to-8-membered carbocycle, 3- to 8-membered heterocycle, hydrogen, deuterium or halogen; and
s is 1.

In certain embodiments, the compound is selected from the group consisting of:

TABLE 8
Additional Compounds.

| | |
|---|---|
| 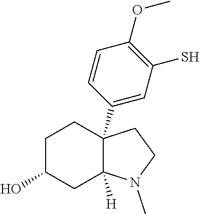 [SMILES: COc1ccc([C@@]23CC[C@@H](O)C[C@@H]2N(C)CC3)cc1S] | 1031 |
| 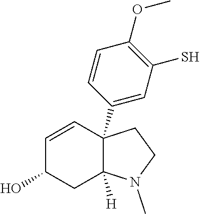 [SMILES: COc1ccc([C@@]23C=C[C@@H](O)C[C@@H]2N(C)CC3)cc1S] | 1032 |
| 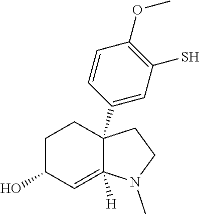 | 1033 |
| 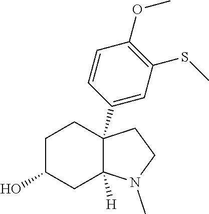 [SMILES: COc1ccc([C@@]23CC[C@@H](O)C[C@@H]2N(C)CC3)cc1SC] | 1034 |
| 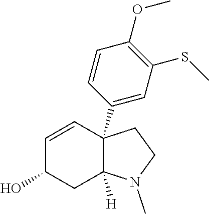 [SMILES: COc1ccc([C@@]23C=C[C@@H](O)C[C@@H]2N(C)CC3)cc1SC] | 1035 |
| 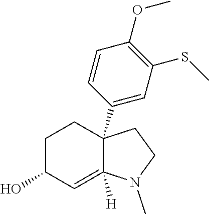 | 1036 |
| 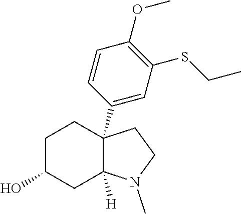 [SMILES: CCSc1cc([C@@]23CC[C@@H](O)C[C@@H]2N(C)CC3)ccc1OC] | 1037 |
| 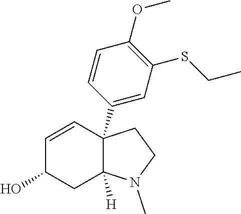 [SMILES: CCSc1cc([C@@]23C=C[C@@H](O)C[C@@H]2N(C)CC3)ccc1OC] | 1038 |
| 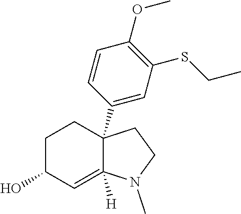 | 1039 |
| 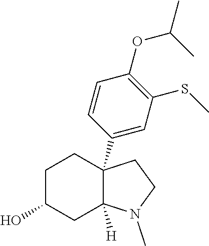 [SMILES: CSc1cc([C@@]23CC[C@@H](O)C[C@@H]2N(C)CC3)ccc1OC(C)C] | 1040 |
| 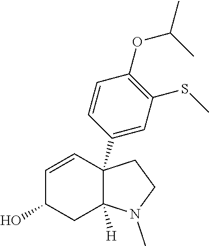 [SMILES: CSc1cc([C@@]23C=C[C@@H](O)C[C@@H]2N(C)CC3)ccc1OC(C)C] | 1041 |
| 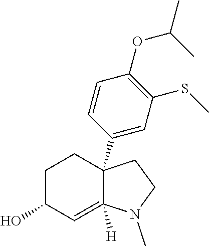 | 1042 |
| 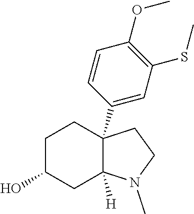 [SMILES: COc1ccc([C@@]23CC[C@@H](O)C[C@@H]2N(C)CC3)cc1SC] | 1043 |
| 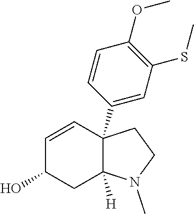 [SMILES: COc1ccc([C@@]23C=C[C@@H](O)C[C@@H]2N(C)CC3)cc1SC] | 1044 |
| 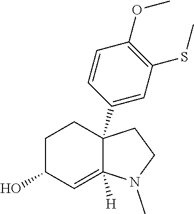 | 1045 |
| 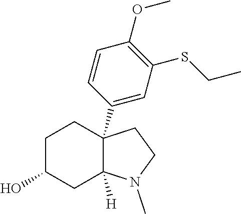 [SMILES: CCSc1cc([C@@]23CC[C@@H](O)C[C@@H]2N(C)CC3)ccc1OC] | 1046 |
| 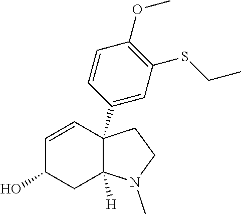 [SMILES: CCSc1cc([C@@]23C=C[C@@H](O)C[C@@H]2N(C)CC3)ccc1OC] | 1047 |
| 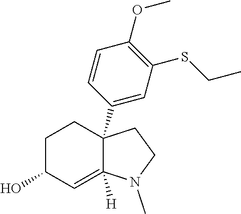 | 1048 |
| 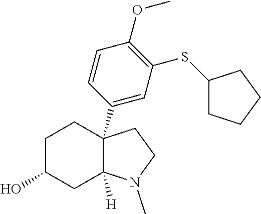 [SMILES: COc1ccc([C@@]23CC[C@@H](O)C[C@@H]2N(C)CC3)cc1SC1CCCC1] | 1049 |
| 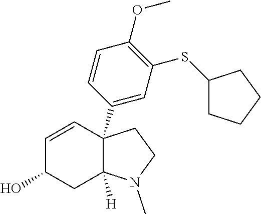 [SMILES: COc1ccc([C@@]23C=C[C@@H](O)C[C@@H]2N(C)CC3)cc1SC1CCCC1] | 1050 |
| 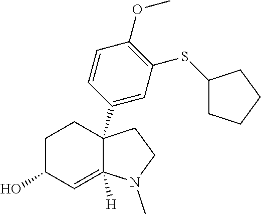 | 1051 |

or a pharmaceutically acceptable salt thereof.

In certain embodiments, the compound is selected from the group consisting of:

TABLE 9
Additional Compounds.

| | |
|---|---|
| 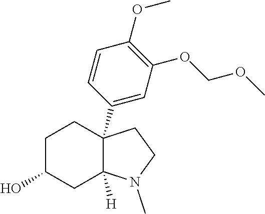 [SMILES: COCOc1cc([C@@]23CC[C@@H](O)C[C@@H]2N(C)CC3)ccc1OC] | 1052 |
| 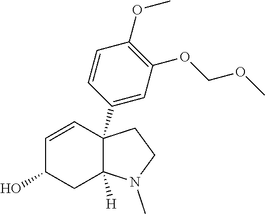 [SMILES: COCOc1cc([C@@]23C=C[C@@H](O)C[C@@H]2N(C)CC3)ccc1OC] | 1053 |
| 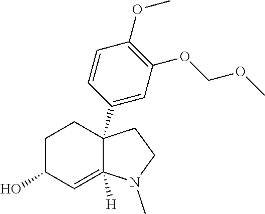 | 1054 |
| 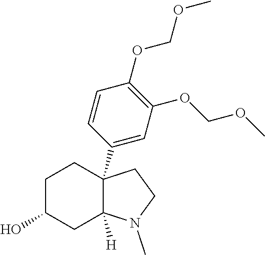 [SMILES: COCOc1ccc([C@@]23CC[C@@H](O)C[C@@H]2N(C)CC3)cc1OCOC] | 1055 |
| 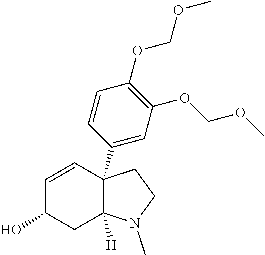 [SMILES: COCOc1ccc([C@@]23C=C[C@@H](O)C[C@@H]2N(C)CC3)cc1OCOC] | 1056 |
| 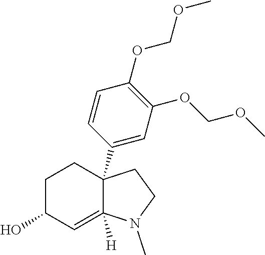 | 1057 |
| 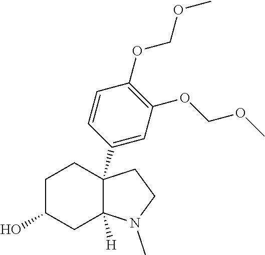 [SMILES: COCOc1ccc([C@@]23CC[C@@H](O)C[C@@H]2N(C)CC3)cc1OCOC] | 1058 |
| 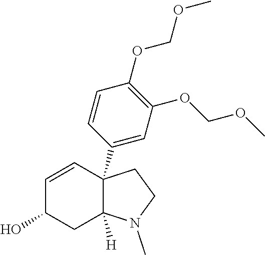 [SMILES: COCOc1ccc([C@@]23C=C[C@@H](O)C[C@@H]2N(C)CC3)cc1OCOC] | 1059 |
| 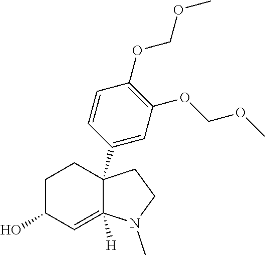 | 1060 |

or a pharmaceutically acceptable salt thereof.

In certain embodiments, the compound is selected from the group consisting of:

TABLE 10
Additional Compounds.

| | |
|---|---|
| 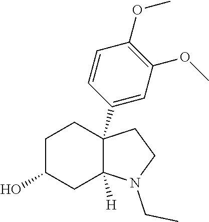 [SMILES: CCN1CC[C@]2(c3ccc(OC)c(OC)c3)CC[C@@H](O)C[C@H]12] | 1061 |
| 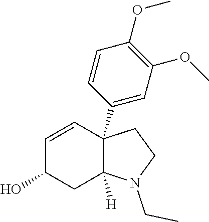 [SMILES: CCN1CC[C@]2(c3ccc(OC)c(OC)c3)C=C[C@@H](O)C[C@H]12] | 1062 |
| 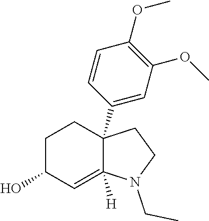 | 1063 |
| 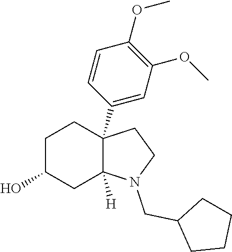 [SMILES: COc1ccc([C@@]23CC[C@@H](O)C[C@@H]2N(CC2CCCC2)CC3)cc1OC] | 1064 |
| 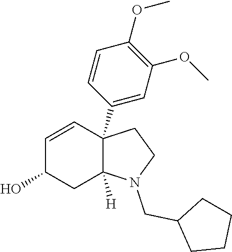 [SMILES: COc1ccc([C@@]23C=C[C@@H](O)C[C@@H]2N(CC2CCCC2)CC3)cc1OC] | 1065 |
| 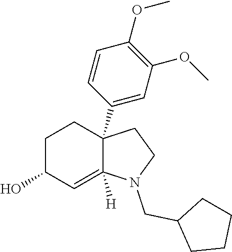 | 1066 |
| 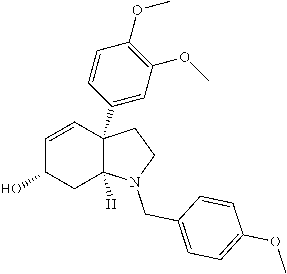 [SMILES: COc1ccc(CN2CC[C@]3(c4ccc(OC)c(OC)c4)C=C[C@@H](O)C[C@H]23)cc1] | 1067 |
| 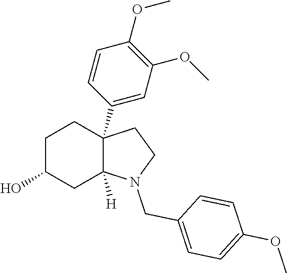 [SMILES: COc1ccc(CN2CC[C@]3(c4ccc(OC)c(OC)c4)CC[C@@H](O)C[C@H]23)cc1] | 1068 |
| 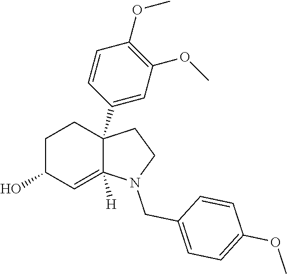 | 1069 |

or a pharmaceutically acceptable salt thereof.

In certain embodiments, the compound is

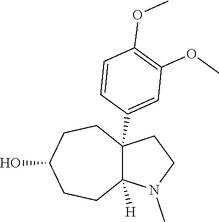 [SMILES: COc1ccc([C@@]23CC[C@@H](O)CC[C@@H]2N(C)CC3)cc1OC]

or a pharmaceutically acceptable salt thereof.

In certain embodiments, the compound is a compound of Formula (VA), (VB) or (VC):

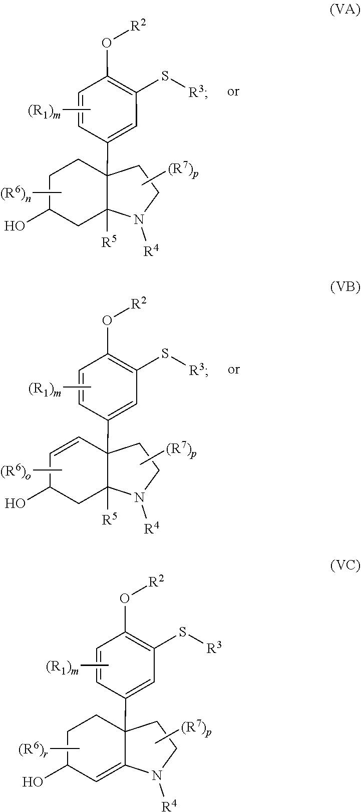

or a pharmaceutically acceptable salt thereof; wherein
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R⁴ is H or alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a};
R⁵ is H, deuterium, or methyl;
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form cycloalkyl, heterocyclyl or heteroaryl, wherein each hydrogen atom in cycloalkyl, heterocyclyl, and 5 heteroaryl is optionally substituted by halo, hydroxy, alkoxy, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, alkyl, —OH, or —NH₂; wherein each hydrogen atom in alkyl is optionally substituted by —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is a compound of Formula (VIA), (VIB) or (VIC):

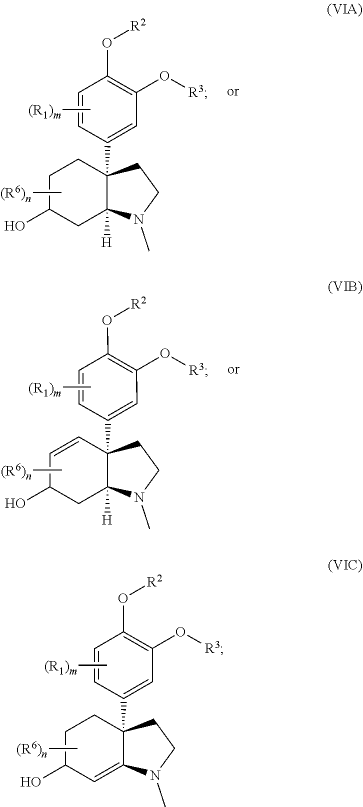

or a pharmaceutically acceptable salt thereof, wherein
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b};
or two R's on a single carbon atom together with the carbon atom to which they are attached combine to form cycloalkyl, heterocyclyl or heteroaryl, wherein each hydrogen atom in cycloalkyl, heterocyclyl, and 5 heteroaryl is optionally substituted by halo, hydroxy, alkoxy, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
m is 0, 1, 2, or 3;
n is 0, 1, 2, 3, 4, 5, or 6;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is a compound of Formula (VIA), (VIB) or (VIC):

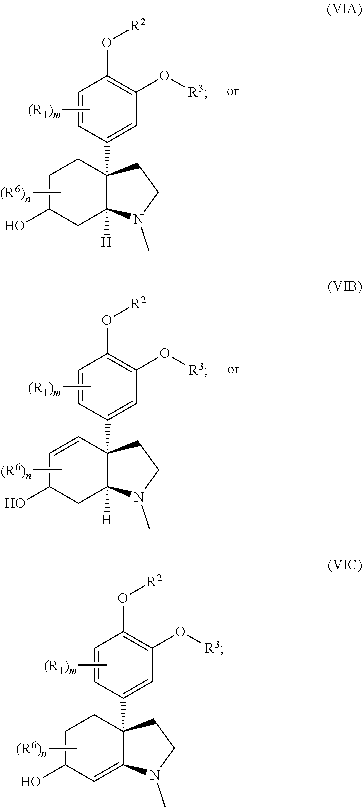

or a pharmaceutically acceptable salt thereof; wherein
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
two R⁶s on different carbon atoms together with the carbon atoms to which they are attached combine to form a bridging cycloalkyl or heterocyclyl, wherein each hydrogen atom in cycloalkyl, or heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
m is 0, 1, 2, or 3;
n is 2;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is a compound of Formula (VII):

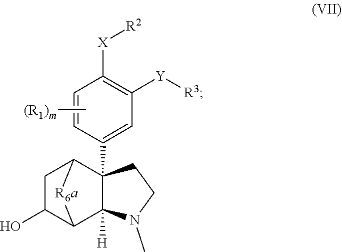

or a pharmaceutically acceptable salt thereof, wherein
X and Y are each independently O or S;
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R₆ₐ is alkyl forming a bridging cycloalkyl or heteroalkyl comprising a N, S, or O heteroatom to form a bridging heterocyclyl, wherein each hydrogen atom in cycloalkyl, or heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
m is 0, 1, 2, or 3;
n is 2;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is a compound of Formula (VII), or a pharmaceutically acceptable salt thereof; wherein
X and Y are each independently O;
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R₆ₐ is C₁-C₄ alkyl forming a bridging cycloalkyl or 1-4 atom heteroalkyl comprising a N, S, or O heteroatom to form a bridging heterocyclyl, wherein each hydrogen atom in cycloalkyl, or heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
m is 0, 1, 2, or 3;
n is 2;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is a compound of Formula (VII-A):

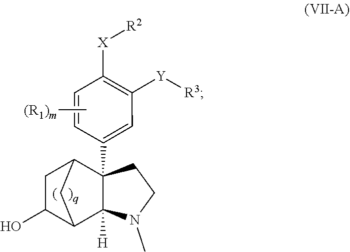

or a pharmaceutically acceptable salt thereof, wherein
X and Y are each independently O or S;
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
m is 0, 1, 2, or 3;
q is 0, 1, 2, or 3;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is a compound of Formula (VII-A):

or a pharmaceutically acceptable salt thereof; wherein X and Y are each O and m is 0.

In certain embodiments, the compound is a compound of Formula (VII-A-1) or Formula (VII-A-2)

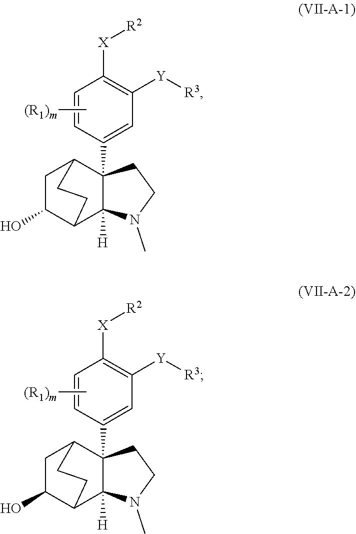

or a pharmaceutically acceptable salt thereof; wherein
X and Y are each independently O or S;
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
m is 0, 1, 2, or 3;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is a compound of Formula (VII-A), Formula (VII-A-1) or Formula (VII-A-2), or a pharmaceutically acceptable salt thereof; wherein
X and Y are each O;
R¹ is halogen, C₁₋₄ alkyl, C₁₋₄ alkoxy, C₃₋₆ cycloalkyl, 3-6 membered heterocycle comprising a N, O or S heteroatom, —C(O)NR^{a}R^{b}, cyano, or C₂₋₄ alkenyl; wherein the C₁₋₄ alkyl is optionally substituted with the optionally substituted with one or more halogen, C₁₋₄ alkoxy, C₃₋₆ cycloalkyl, 3-6 membered heterocycle comprising a N, O or S heteroatom; each R^{a} and R^{b} is independently H, or C₁₋₄ alkyl;
each of R² and R³ is independently H, halogen, C₁₋₄ alkyl, C₁₋₄ alkoxy, C₃₋₆ cycloalkyl, 3-6 membered heterocycle comprising a N, O or S heteroatom, —C(O)NR^{a}R^{b}, cyano, or C₂₋₄ alkenyl; wherein the C₁₋₄ alkyl is optionally substituted with the optionally substituted with one or more halogen, C₁₋₄ alkoxy, C₃₋₆ cycloalkyl, 3-6 membered heterocycle comprising a N, O or S heteroatom; each R^{a} and R^{b} is independently H, or C₁₋₄ alkyl;
each R^{a} and R^{b} is independently H, or C₁₋₄ alkyl; and
m is 0, 1, 2 or 3.

In certain embodiments, the compound is a compound of Formula (IX-A), (IX—B) or (IX—C)

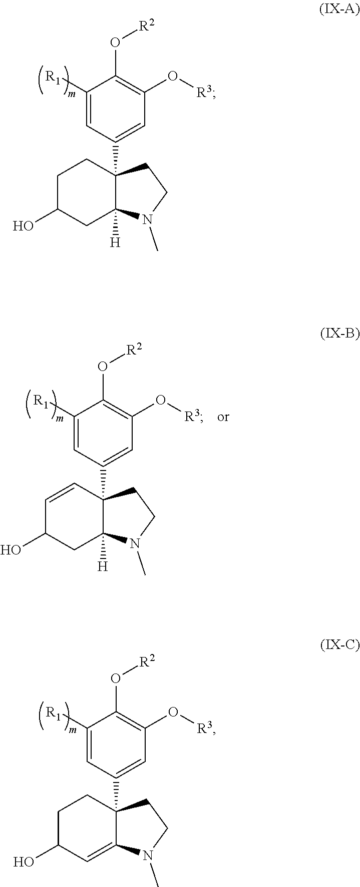

or a pharmaceutically acceptable salt thereof; wherein
R¹ is halo, methoxy, cyclopropyl, amido or acetyl;
m is 0 or 1;
each of R² and R³ is independently H, C₂₋₄ alkenyl, C₂₋₄ alkynyl, C₃₋₈ cycloalkyl, C₁₋₄ alkyl, C₁₋₄ haloalkyl, OR^{a}, C₃₋₈ cycloalkyl, or phenyl; or
R² and R³ together with the atoms to which they are attached combine to form a 5-6 membered heterocyclyl comprising one or more N, O or S heteroatoms wherein each hydrogen atom in heterocyclyl is optionally substituted by halo, C₁₋₄ alkyl, C₁₋₄ haloalkyl, or OR^{a}; and
R^{a} is C₁₋₄ alkyl.

In certain embodiments, the compound is a compound of Formula (X-A), (X—B) or (X—C)

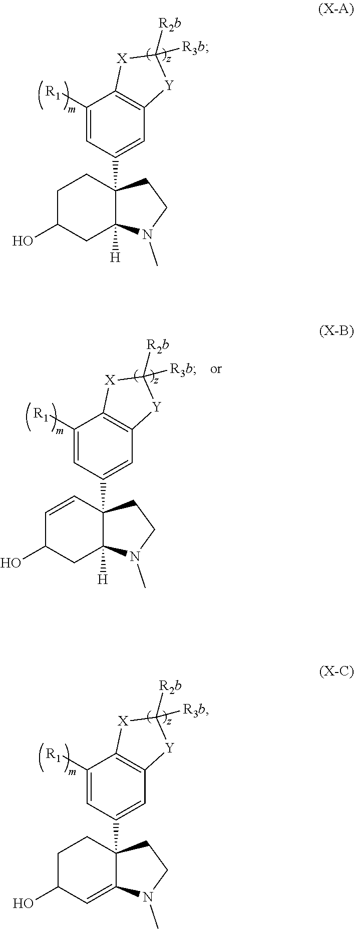

pharmaceutically acceptable salt thereof; wherein
X and Y are each O, NR^{a} or S;
R¹ is halo, methoxy, cyclopropyl, amido or acetyl;
m is 0 or 1;
z is 1 or 2;
each R^{2b} and R^{3b} is independently H, halo, C₁₋₄ alkyl, C₁₋₄ haloalkyl, OR^{a}, and C₃₋₈ cycloalkyl; and
each R^{a} is independently H or C₁₋₄ alkyl.

In certain embodiments, the compound is a compound of Formula (X-A), (X—B) or (X—C) pharmaceutically acceptable salt thereof, wherein X and Y are each O, m is 0 or 1, z is 1, and each R^{2b} and R^{3b} is independently H or F.

### Methods of Treatment

In some embodiments, methods of treating a patient suffering from a disease comprise administering to a patient an effective amount of a composition comprising a compound disclosed herein for the treatment or prevention of a mental health disorder. In some embodiments, methods of treating a patient suffering from a disease comprise administering to a patient an effective amount of a composition comprising a compound disclosed herein for the treatment or prevention of a diagnosed condition selected from anxiety and depression. In some embodiments, a method comprises the administration to a patient in need thereof of a therapeutically effective amount of a compound of Formula (IA), (IB), and (IC) for the treatment of depression. In some embodiments, a method comprises the administration to a patient in need thereof of a therapeutically effective amount of a compound of Formula (IA), (IB), and (IC) for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of Formula (IA), (IB), and (IC) for the treatment of anxiety and hysteria or anxiety and depression.

The present disclosure provides methods of treating subjects diagnosed with various neurological and psychiatric disorders by administering to said subjects a dose of a pharmaceutical composition comprising a compound provided herein. Said disorders include, without limitation, attention deficit disorder hyperactivity disorder (ADHD), cognition impairment, anxiety disorders, especially generalized anxiety disorder (GAD), panic disorder, bipolar disorder, also known as manic depression or manic-depressive disorder, obsessive compulsive disorder (OCD), posttraumatic stress disorder (PTSD), acute stress disorder, social phobia, simple phobia, pre-menstrual dysphoric disorder (PMDD), social anxiety disorder (SAD), major depressive disorder (MDD), supranuclear palsy, eating disorders, especially obesity, anorexia nervosa, bulimia nervosa, and binge eating disorder, analgesia (including neuropathic pain, especially diabetic neuropathy), substance abuse disorders (including chemical dependencies) like nicotine addiction, cocaine addiction, alcohol and amphetamine addiction, Lesch-Nyhan syndrome, neurodegenerative diseases like Parkinson [symbol] disease, late luteal phase syndrome or narcolepsy, psychiatric symptoms anger such as, rejection sensitivity, movement disorders, like extrapyramidal syndrome, Tic disorders and restless leg syndrome (RLS), tardive dyskinesia, supranuclear palsy, sleep related eating disorder (SRED), night eating syndrome (NES), urinary incontinence (including stress urinary incontinence (SUI) and mixed incontinence), migraine, fibromyalgia syndrome (FS), chronic fatigue syndrome (CFS), sexual dysfunction especially premature ejaculation and male impotence, thermoregulatory disorders (e.g., hot flashes that may be associated with menopause), and lower back pain.

In some embodiments, methods of treating a disease or disorder comprise the administration of a therapeutically effective amount of a compound disclosed herein, wherein the disease or disorder is selected from the group consisting of major depressive disorder, social anxiety disorder, obsessive compulsive disorder (OCD), panic disorder (PD), generalized anxiety disorder (GAD), posttraumatic stress disorder (PTSD), bulimia nervosa, premenstrual dysphoric disorder (PMDD), premature ejaculation, arthritis, chronic fatigue, multiple sclerosis, lupus, irritable bowel syndrome (IBS), migraine headache, diabetic neuropathy, fibromyalgia, attention-deficit/hyperactivity disorder (ADHD), autistic spectrum disorders, bipolar depression, attention deficit disorder, chronic pain, neurocardiogenic syncope, post traumatic stress disorders, obsessive compulsive disorders, anxiety, panic attacks, pain, neuralgic pain, postherpetic neuralgia, phobias of various types, and eating disorders.

In some embodiments, methods of treating a disease or disorder comprise the administration of a therapeutically effective amount of a compound disclosed herein, wherein the disease or disorder is selected from the group consisting of lower back pain, attention deficit hyperactivity disorder (ADHD), cognition impairment, anxiety disorders, generalized anxiety disorder (GAD), panic disorder, bipolar disorder or manic depression or manic-depressive disorder, obsessive compulsive disorder (OCD), posttraumatic stress disorder (PTSD), acute stress disorder, social phobia, simple phobias, pre-menstrual dysphoric disorder (PMDD), social anxiety disorder (SAD), major depressive disorder (MDD), postnatal depression, dysthymia, depression associated with Alzheimer [symbol] disease, Parkinson [symbol] disease, or psychosis, supranuclear palsy, eating disorders, obesity, anorexia nervosa, bulimia nervosa, binge eating disorder, analgesia, substance abuse disorders, chemical dependencies, nicotine addiction, cocaine addiction, alcohol and amphetamine addiction, Lesch-Nyhan syndrome, neurodegenerative diseases, Parkinson [symbol] disease, late luteal phase syndrome or narcolepsy, psychiatric symptoms, anger, rejection sensitivity, movement disorders, extrapyramidal syndrome, Tic disorders, restless leg syndrome (RLS), tardive dyskinesia, supranuclear palsy, sleep related eating disorder (SRED), night eating syndrome (NES), stress urinary incontinence (SUI), migraine, neuropathic pain, diabetic neuropathy, fibromyalgia syndrome (FS), chronic fatigue syndrome (CFS), sexual dysfunction, premature ejaculation, male impotence, and thermoregulatory disorders.

In some embodiments, the compound disclosed herein is administered to the patient in a unit dose. In some embodiments, the compound disclosed herein is prescribed to a patient in an oral unit dose such as a capsule or tablet for administration once or more times per day. In some embodiments, a compound disclosed herein is administered to a patient for the treatment of a disease or condition for which mesembrine is safe and effective for treatment. In some embodiments, a method comprises the administration to a patient in need thereof of a therapeutically effective amount of a compound of Formula (IA), (IB), and (IC) for the treatment of anxiety. In some embodiments, a method comprises the administration to a patient in need thereof of a therapeutically effective amount of a compound of Formula (IA), (IB), and (IC) for the treatment of a disease selected from the group consisting of mild to moderate depression and major depressive episodes. In some embodiments, a method comprises the administration to a patient in need thereof of a therapeutically effective amount of a compound of Formula (IA), (IB), and (IC) for the treatment of a disease selected from the group consisting of psychological and psychiatric disorders where anxiety is present. In some embodiments, a method comprises the administration to a patient in need thereof of a therapeutically effective amount of a compound of Formula (IA), (IB), and (IC) for the treatment of major depressive episodes. In some embodiments, a method comprises the administration to a patient in need thereof of a therapeutically effective amount of a compound of Formula (IA), (IB), and (IC) for the treatment of a disease selected from the group consisting of alcohol and drug dependence, bulimia nervosa, and obsessive-compulsive disorders.

In some embodiments, an amount of from 20 micrograms to 2 milligrams of a compound of Formula (IA), (IB), and (IC) is orally administered to a patient to treat the patient in need thereof. In some embodiments, an amount of from 20 micrograms to 2 milligrams of a compound of Formula (IA), (IB), and (IC) is orally administered to a patient to treat the patient in need thereof. In some embodiments, an amount of from 20 micrograms to 2 grams of a compound of Formula (IA), (IB), and (IC) is orally administered to a patient to treat the patient in need thereof. In some embodiments, an amount of from 20 micrograms to 2 grams of a compound of Formula (IA), (IB), and (IC) is orally administered to a patient to treat the patient in need thereof.

### Pharmaceutical Compositions

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Formula (I) or pharmaceutically acceptable salts thereof can be formulated for oral administration. For example, a compound provided herein can be combined with suitable compendial excipients to form an oral unit dosage form, such as a capsule or tablet, containing a target dose of a compound of Formula (I). The drug product can be prepared by first manufacturing the compound of Formula (I) as an active pharmaceutical ingredient (API), followed by roller compaction/milling with intragranular excipients and blending with extra granular excipients. A Drug Product can contain the selected compound of Formula (I) as the API and excipient components in a tablet in a desired dosage strength of a compound of Formula (1). The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a compound of Formula (I) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient [symbol] condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention.

Pharmaceutically-acceptable salts include, for example, acid-addition salts and base addition salts. The acid that is added to a compound to form an acid-addition salt can be an organic acid or an inorganic acid. A base that is added to a compound to form a base addition salt can be an organic base or an inorganic base. In some embodiments, a pharmaceutically-acceptable salt is a metal salt, in some embodiments, a pharmaceutically-acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al.,

“Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000). All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups, C₁-C₁₀ branched-chain alkyl groups, cycloalkyl (alicyclic) groups, alkyl-substituted cycloalkyl groups, and cycloalkyl-substituted alkyl groups. Preferably, the “alkyl” group refers to C₁-C₇ straight-chain alkyl groups or C₁-C₇ branched-chain alkyl groups. Most preferably, the “alkyl” group refers to C₁-C₃ straight-chain alkyl groups or C₁-C₃ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “haloalkyl” refers to an alkyl group substituted with at least one hydrogen atom on a carbon replaced by a halogen. Illustrative halogens include fluoro, chloro, bromo, and iodo. Illustrative haloalkyl groups include trifluoromethyl and 2,2,2-trifluoroethyl, etc.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

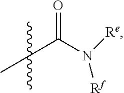

wherein R^{e} and R^{f} each independently represent a hydrogen or hydrocarbyl group, or R^{e} and R^{f} taken together with the N atom to which they are attached complete a heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Preferably, the “alkoxy” group refers to C₁-C₇ straight-chain alkoxy groups or C₁-C₇ branched-chain alkoxy groups. Representative alkoxy groups include methoxy, ethoxy, propoxy, tert-butoxy and the like.

The term “aryloxy” refers to an aryl group having an oxygen attached thereto. Preferably, the “aryloxy” group refers to C₆-C₁₀ aryloxy groups or 5-7-membered heteroaryloxy groups. Representative aryloxy groups include phenoxy (C₆H₅—O—) and the like.

The terms “amine” and “amino” are art-recognized and refer to both unsubstituted and substituted amines and salts thereof, e.g., a moiety that can be represented by

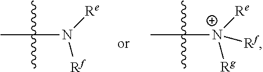

wherein R^{e}, R^{f}, and R^{g}, each independently represent a hydrogen or a hydrocarbyl group, or R^{c} and R taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring, for example a phenyl. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

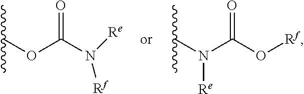

wherein R^{e} and R^{f} independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, “and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains six or fewer carbon atoms, preferably four or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

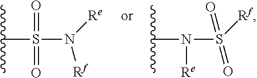

wherein R^{e} and R^{f} independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR^{e} or —SC(O)R^{e} wherein R^{c} represents a hydrocarbyl.

The term “thioether”, as used herein, is equivalent to an ether, wherein the oxygen is replaced with a sulfur.

The term “urea” is art-recognized and may be represented by the general formula

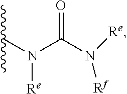

wherein R^{e} and R^{f} independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. The mono- or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form mesembrine. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs include using ester or phosphoramidate as biologically labile or cleavable (protecting) groups. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

Unless otherwise indicated in the tables of compounds herein, the abbreviation RAC or rac indicates a racemic mixture, and DIAST indicates a specific diastereomer. In illustrative embodiments, although a compound may be depicted with [symbol] or [symbol] bonds, such a depiction may be denoting relative stereochemistry based on elution peaks from a chiral separation.

### Additional Embodiments

In certain embodiments, the compound is a compound of one or more of the following embodiments, or a pharmaceutically acceptable salt thereof:

1. A compound of Formula (IA), (IB) or (IC):

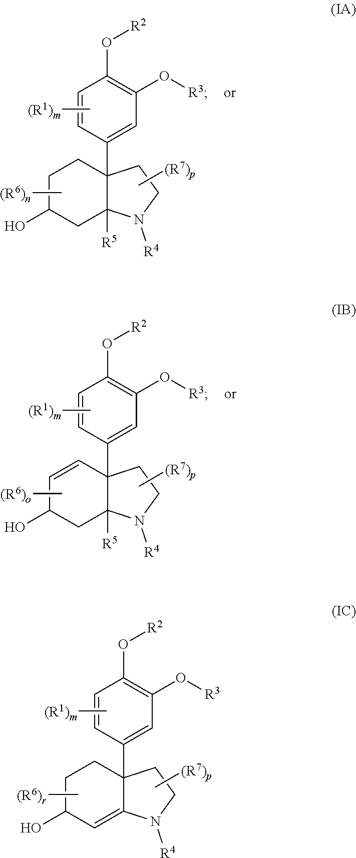

or a pharmaceutically acceptable salt thereof; wherein
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R⁴ is H or alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a};
R⁵ is H, deuterium, or methyl;
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form cycloalkyl, heterocyclyl or heteroaryl, wherein each hydrogen atom in cycloalkyl, heterocyclyl, and 5 heteroaryl is optionally substituted by halo, hydroxy, alkoxy, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, alkyl, —OH, or —NH₂; wherein each hydrogen atom in alkyl is optionally substituted by —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl;
provided the compound is not:

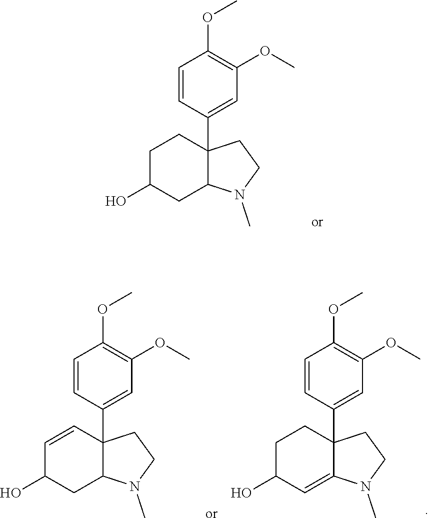 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

2. The compound of embodiment 1, wherein the compound is of Formula (IA-a), (IA-b), (IB-a), (IB-b), (IC-a) or (IC-b):

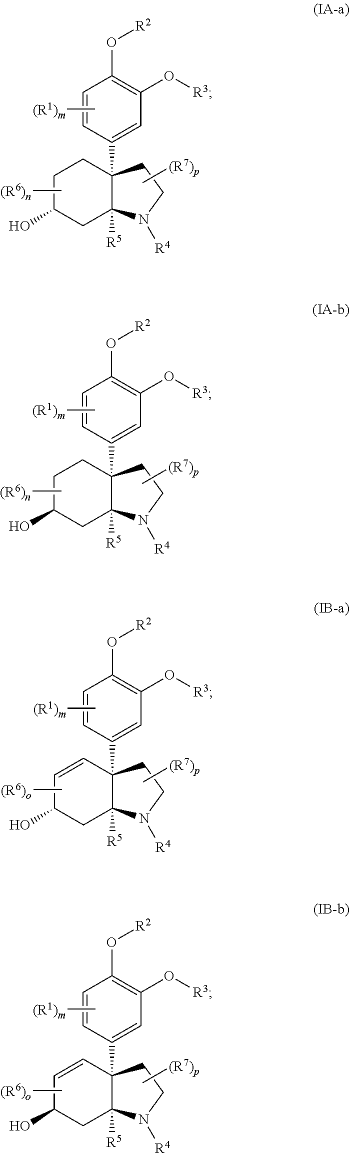

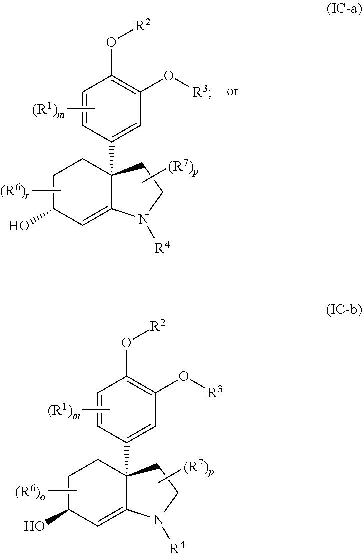

or a pharmaceutically acceptable salt thereof; provided the compound is not:

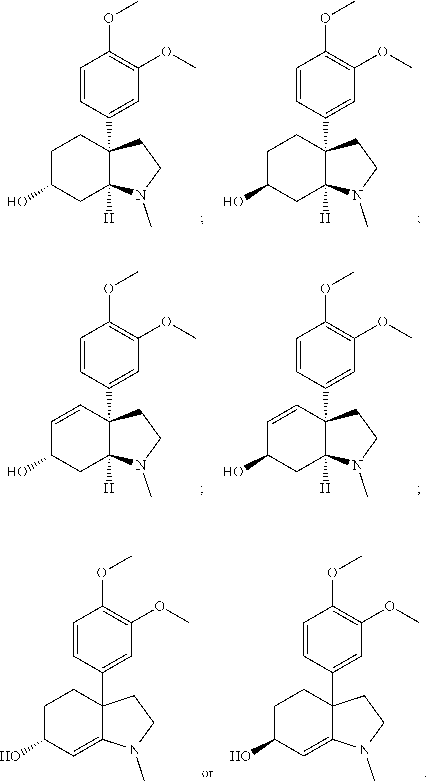 [SMILES: COc1ccc([C@@]23CC[C@@H](O)C[C@@H]2N(C)CC3)cc1OC ; COc1ccc([C@@]23CC[C@H](O)C[C@@H]2N(C)CC3)cc1OC ; COc1ccc([C@@]23C=C[C@@H](O)C[C@@H]2N(C)CC3)cc1OC ; COc1ccc([C@@]23C=C[C@H](O)C[C@@H]2N(C)CC3)cc1OC ; COc1ccc(C23CC[C@@H](O)C=C2N(C)CC3)cc1OC ; COc1ccc(C23CC[C@H](O)C=C2N(C)CC3)cc1OC]

3. The compound of embodiment 1, wherein the compound is of Formula (IA-a), (IA-b), (IB-a), (IB-b), (IC-a) or (IC-b):

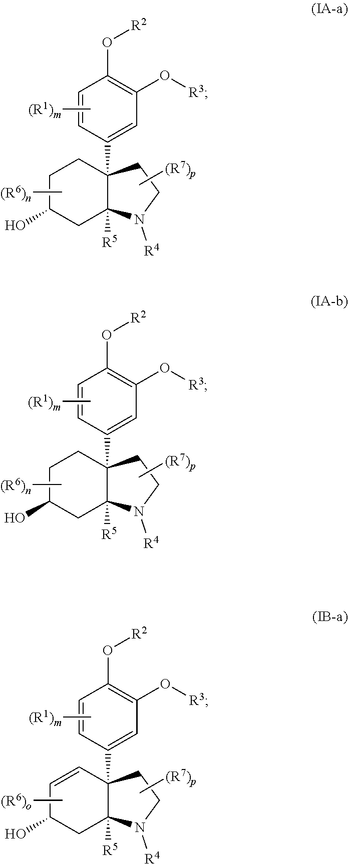

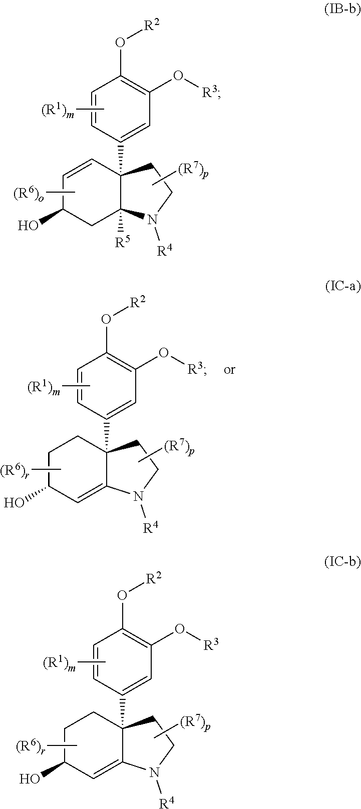

or a pharmaceutically acceptable salt thereof;
wherein the compounds of Formula (IA-a), (IA-b), (IB-a), (IB-b), (IC-a) and (IC-b) have the absolute stereochemistry shown; provided the compound is not:

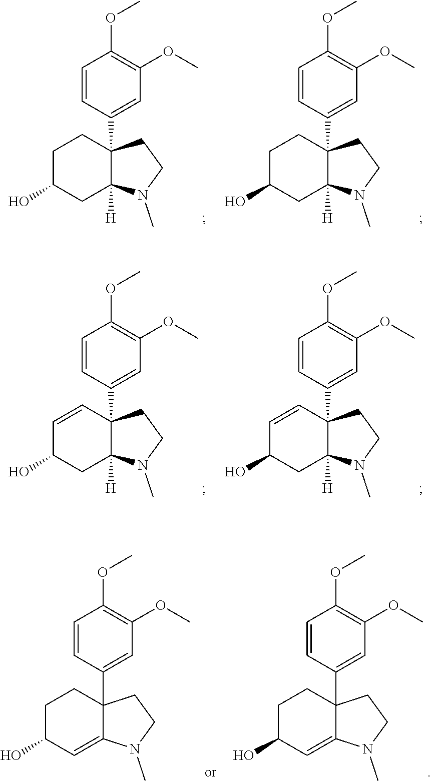 [SMILES: COc1ccc([C@@]23CC[C@@H](O)C[C@@H]2N(C)CC3)cc1OC ; COc1ccc([C@@]23CC[C@H](O)C[C@@H]2N(C)CC3)cc1OC ; COc1ccc([C@@]23C=C[C@@H](O)C[C@@H]2N(C)CC3)cc1OC ; COc1ccc([C@@]23C=C[C@H](O)C[C@@H]2N(C)CC3)cc1OC ; COc1ccc(C23CC[C@@H](O)C=C2N(C)CC3)cc1OC ; COc1ccc(C23CC[C@H](O)C=C2N(C)CC3)cc1OC]

4. The compound of embodiment 3, wherein the compound is of Formula (IA), Formula (IA-a) or Formula (IA-b).

5. The compound of embodiment 3, wherein the compound is of Formula (IB), Formula (IB-a) or Formula (IB-b).

6. The compound of embodiment 3, wherein the compound is of Formula (IC), Formula (IC-a) or Formula (IC-b).

7. The compound of any one of embodiments 1-6, wherein R⁵ is H or methyl.

8. The compound of any one of embodiments 1-6, wherein R⁵ is H.

9. The compound of embodiment 1, wherein the compound is of Formula (IA-1) or (IB-1):

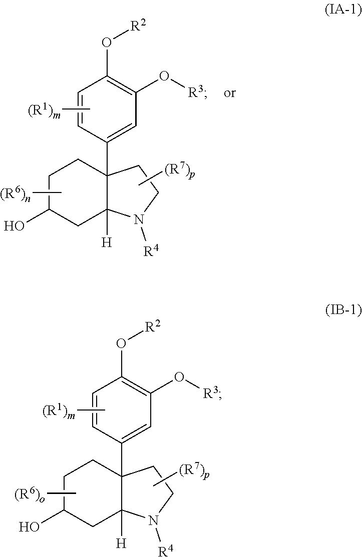

or a pharmaceutically acceptable salt thereof.

10. The compound of embodiment 9, wherein the compound is of Formula (IA-1a), (IA-1b), (IB-1a), or (IB-1b):

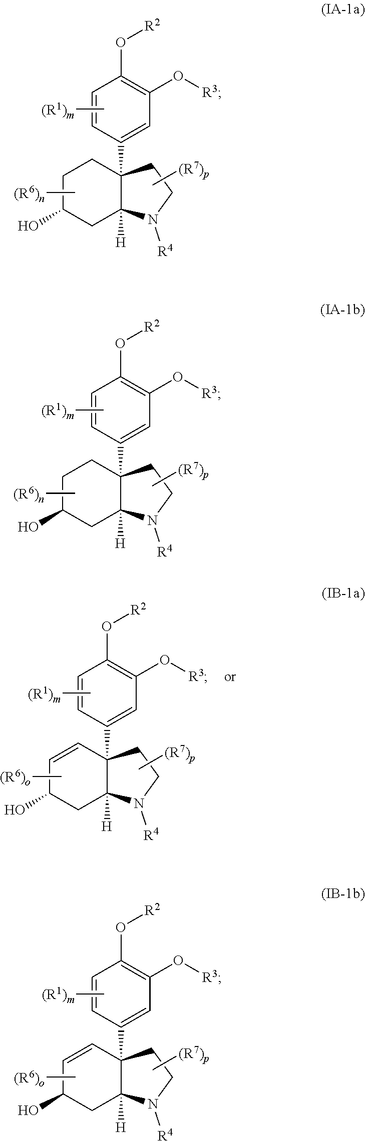

or a pharmaceutically acceptable salt thereof.

11. The compound of embodiment 10, wherein the compound is of Formula (IA-1a), (IA-1b), (IB-1a), or (IB-1b):

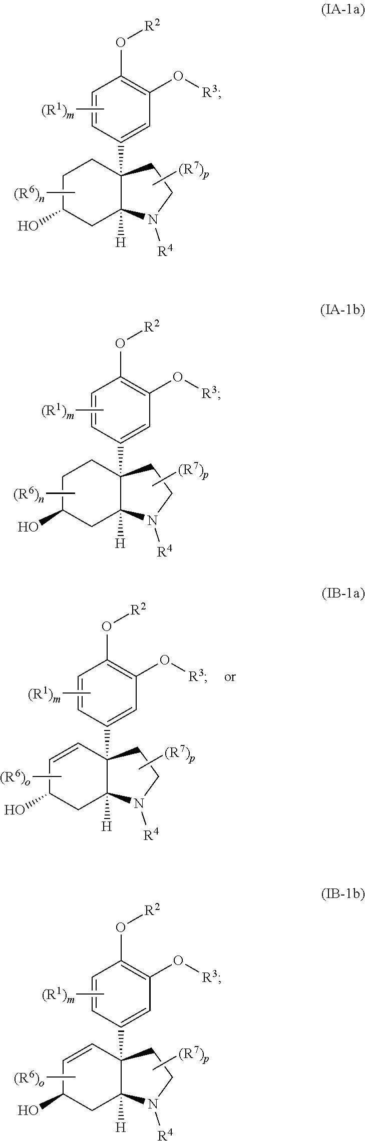

or a pharmaceutically acceptable salt thereof, wherein the compounds of Formula (IA-1a), (IA-1b), (IB-1a), and (IB-1b) have the absolute stereochemistry shown.

12. The compound of embodiment 11, wherein the compound is of Formula (IA-1a).

13. The compound of embodiment 11, wherein the compound is of Formula (IA-1b).

14. The compound of embodiment 11, wherein the compound is of Formula (IB-1a).

15. The compound of embodiment 11, wherein the compound is of Formula (IB-1b).

16. The compound of any one of the preceding embodiments, wherein p is 1; and R⁷ is C₁-C₆ alkyl.

17. The compound of any one of embodiments 1-15, wherein p is 0.

18. The compound of embodiment 1, wherein the compound is of Formula (IA-2), (IB-2) or (IC-2):

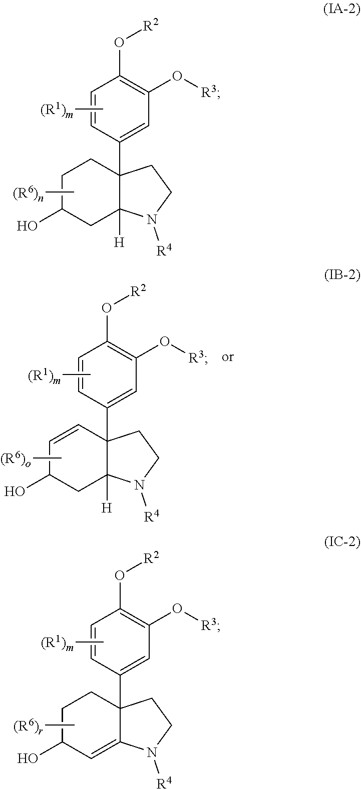

or a pharmaceutically acceptable salt thereof.

19. The compound of embodiment 18, wherein the compound is of Formula (IA-2a), (IA-2b), (IB-2a), (IB-2b), (IC-2a) or (IC-2b):

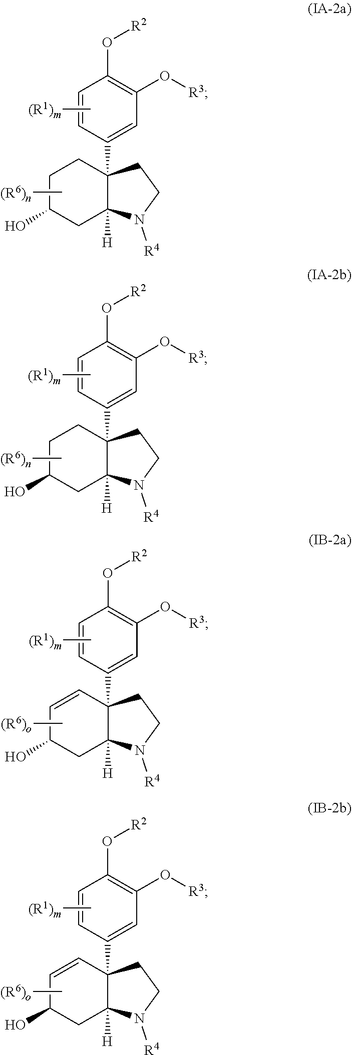

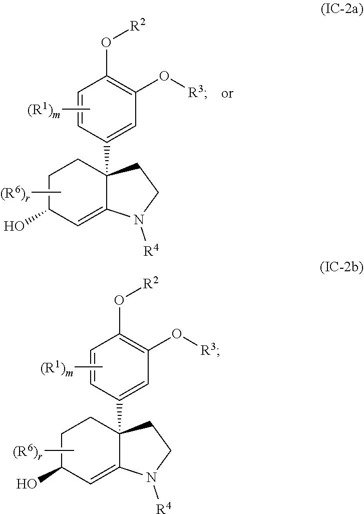

or a pharmaceutically acceptable salt thereof.

20. The compound of embodiment 19, wherein the compound is of Formula (IA-2a), (IA-2b), (IB-2a), (IB-2b), (IC-2a) or (IC-2b):

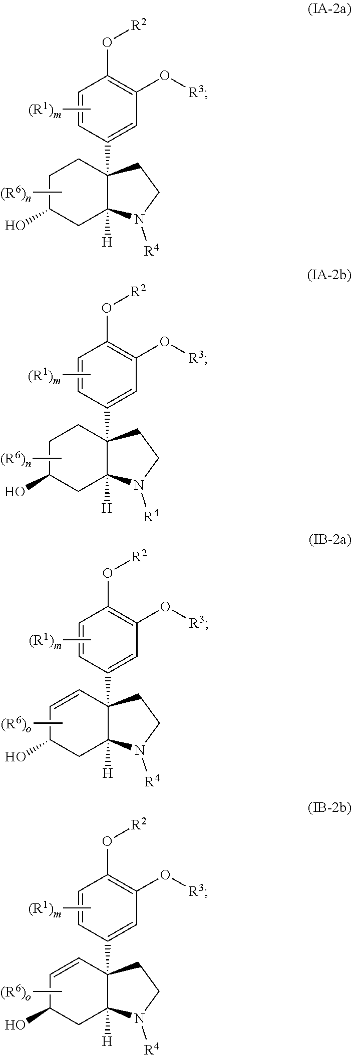

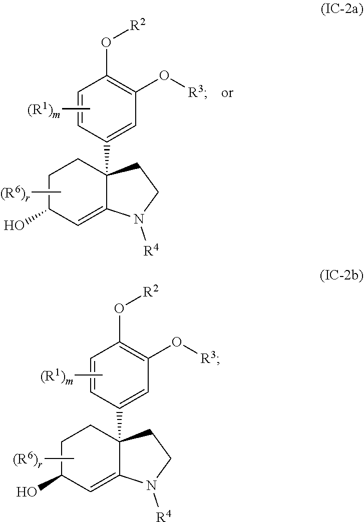

or a pharmaceutically acceptable salt thereof, wherein the compounds of Formula (IA-2a), (IA-2b), (IB-2a), (IB-2b), (IC-2a), and (IC-2b) have the absolute stereochemistry shown.

21. The compound of embodiment 19, wherein the compound is of Formula (IA-2), Formula (IA-2a) or Formula (IA-2b).

22. The compound of embodiment 19, wherein the compound is of Formula (IB-2), Formula (IB-2a) or Formula (IB-2b).

23. The compound of embodiment 19, wherein the compound is of Formula (IC-2), Formula (IC-2a) or Formula (IC-2b).

24. The compound of any one of embodiments 1-19, 22, and 23, wherein n is 1, 2, 3, 4, 5, or 6.

25. The compound of any one of embodiments 1-18, 20, 22, and 23, wherein o is 1, 2, 3, or 4.

26. The compound of any one of embodiments 1-18 and 21-23, wherein r is 1, 2, 3, 4, or 5.

27. The compound of any one of the preceding embodiments, wherein R⁶ is deuterium, C₁-C₆ alkyl, —Si(C₁-C₆ alkyl)₃, phenyl, or —C(O)C₁-C₆ alkyl.

28. The compound of any one of the preceding embodiments, wherein R⁶ is CF₃.

29 The compound of any one of embodiments 1-26, wherein two R⁶s on a single carbon atom together with the carbon atom to which they are attached combine to form C₃-C₆ cycloalkyl.

30. The compound of any one of embodiments 1-26, wherein two R⁶s on different carbon atoms together with the carbon atoms to which they are attached combine to form a C₃-C₈ cycloalkyl, or 3-7 membered heterocyclyl.

31. The compound of embodiment 1, wherein the compound is of Formula (IA-3), (IB-3) or (IC-3):

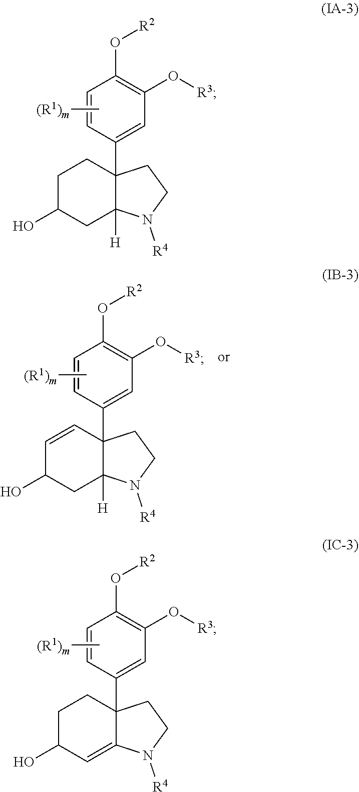

or a pharmaceutically acceptable salt thereof.

32. The compound of embodiment 31, wherein the compound is of Formula (IA-3a), (IA-3b), (IB-3a), (IB-3b) (IC-3a) or (IC-3b):

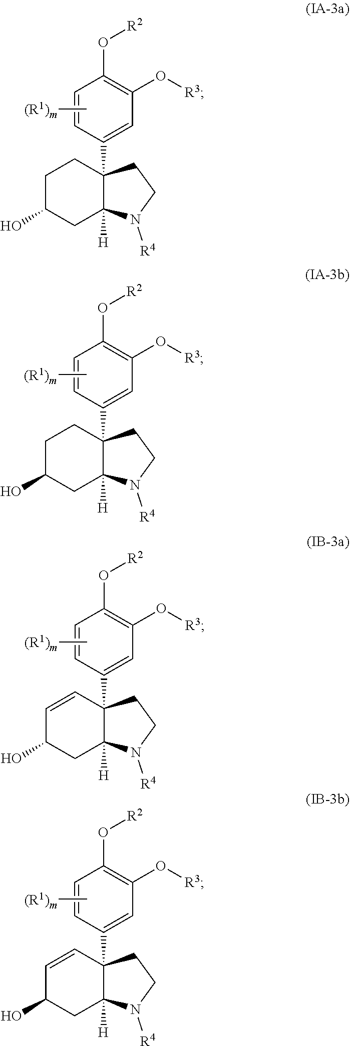

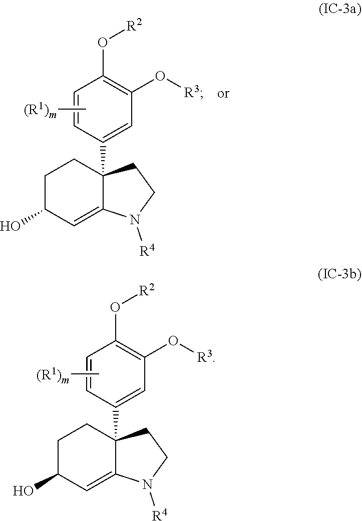

or a pharmaceutically acceptable salt thereof.

33. The compound of embodiment 32, wherein the compound is of Formula (IA-3a), (IA-3b), (IB-33a), (IB-3b) (IC-3a) or (IC-3b):

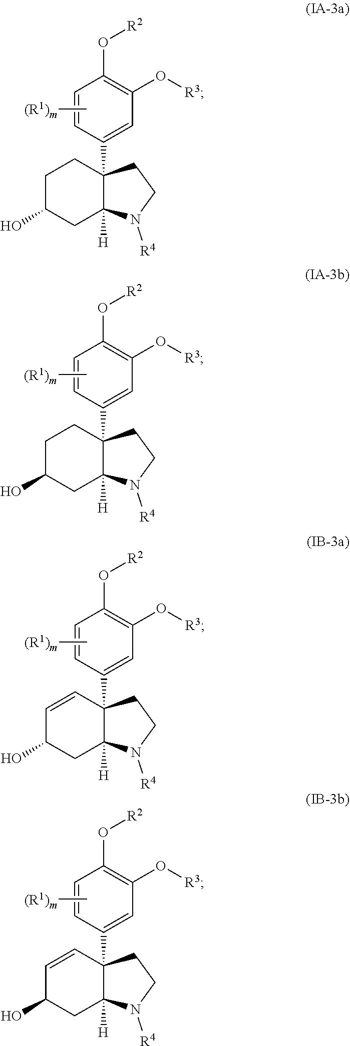

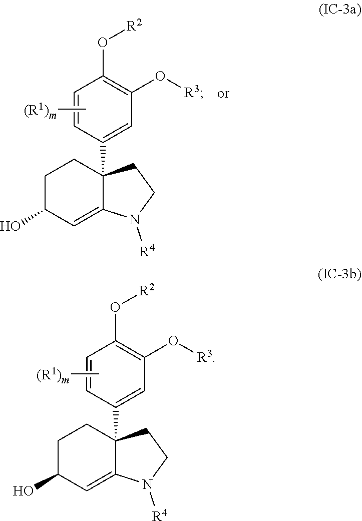

or a pharmaceutically acceptable salt thereof, wherein the compounds of Formula (IA-3a), (IA-3b), (IB-3a), (IB-3b), (IC-3a), and (IC-3b) have the absolute stereochemistry shown.

34. The compound of embodiment 33, wherein the compound is of Formula (IA-3), Formula (IA-3a) or Formula (IA-3b).

35. The compound of embodiment 33, wherein the compound is of Formula (IB-3), Formula (IB-3a) or Formula (IB-3b).

36. The compound of embodiment 33, wherein the compound is of Formula (IC-3), Formula (IC-3a) or Formula (IC-3b).

37. The compound of any one of the preceding embodiments, wherein R⁴ is H or C₁-C₆ alkyl optionally substituted by deuterium, phenyl optionally substituted by —OC₁-C₆ alkyl, or C₃-C₆ cycloalkyl.

38. The compound of any one of the preceding embodiments, wherein R⁴ is methyl or CD₃.

39. The compound of embodiment 1, wherein the compound is of Formula (IA-4), (IB-4) or (IC-4):

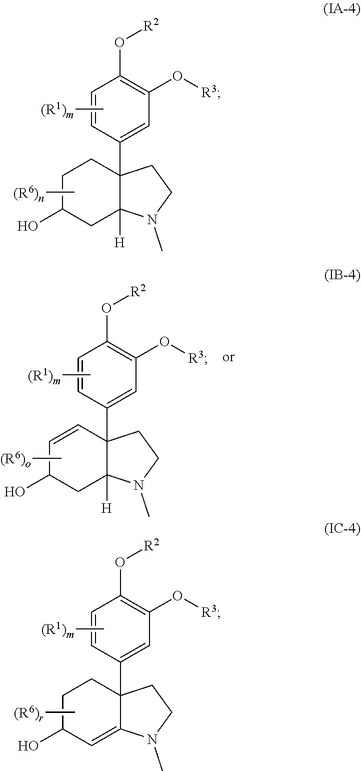

or a pharmaceutically acceptable salt thereof.

40. The compound of embodiment 39, wherein the compound is of Formula (IA-4a), (IA-4b), (IB-4a), (IB-4b), (IC-4a) or (IC-4b):

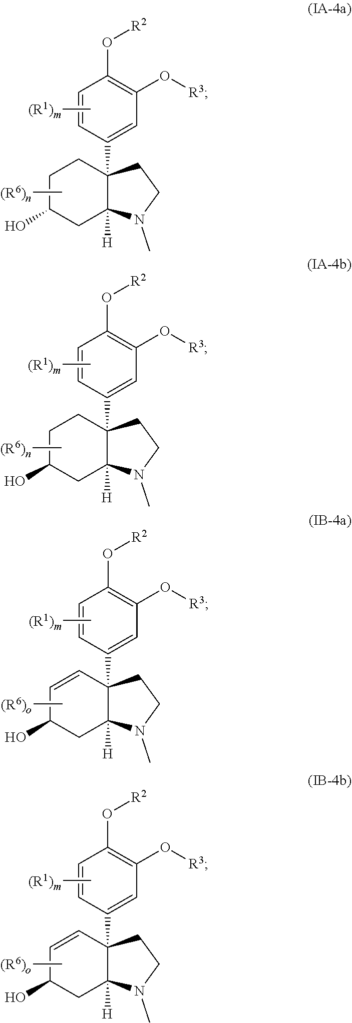

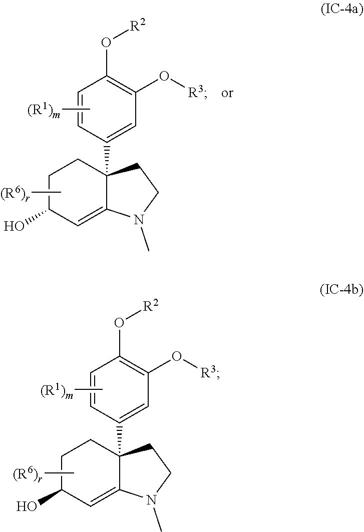

or a pharmaceutically acceptable salt thereof.

41. The compound of embodiment 40, wherein the compound is of Formula (IA-4a), (IA-4b), (IB-4a), (IB-4b), (IC-4a) or (IC-4b):

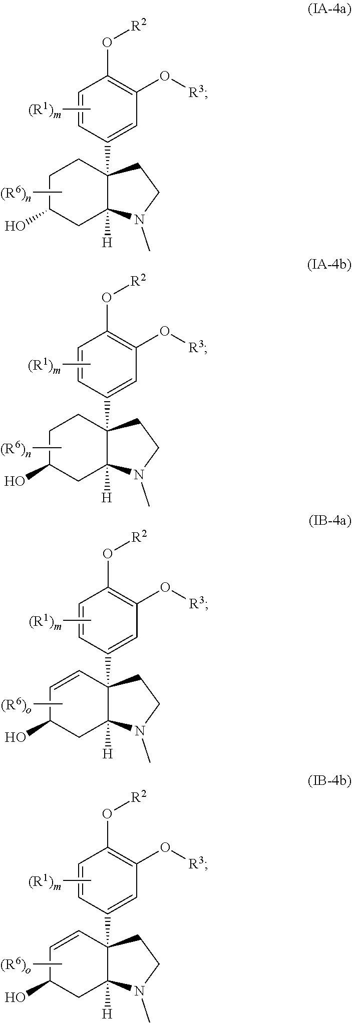

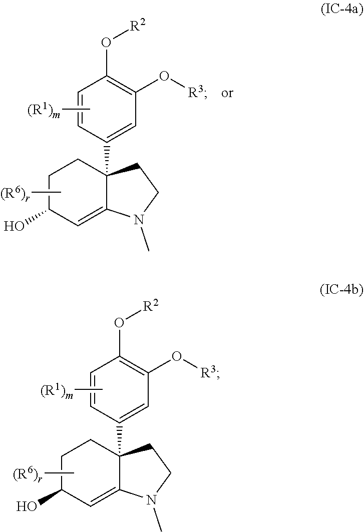

or a pharmaceutically acceptable salt thereof, wherein the compounds of Formula (IA-4a), (IA-4b), (IB-4a), (IB-4b), (IC-4a) and (IC-4b) have the absolute stereochemistry shown.

42. The compound of embodiment 41, wherein the compound is of Formula (IA-4), Formula (IA-4a) or Formula (IA-4b).

43. The compound of embodiment 41, wherein the compound is of Formula (IB-4), Formula (IB-4a) or Formula (IB-4b).

44. The compound of embodiment 41, wherein the compound is of Formula (IC-4), Formula (IC-4a) or Formula (IC-4b).

45. The compound ofany one of the preceding embodiments, wherein m is 1, 2, or 3.

46. The compound of any one of the preceding embodiments, wherein m is 1.

47. The compound of any one of the preceding embodiments, wherein R¹ is halo, alkenyl, cycloalkyl, cyano, or —C(O)NR^{a}R^{b}.

48. The compound of any one of the preceding embodiments, wherein R¹ is fluoro, chloro, bromo, iodo, vinyl, cyclopropyl, cyano, or —C(O)NH₂.

49. The compound of any one of embodiments 39-42, 45, and 46, wherein n is 1, 2, 3, 4, 5, or 6.

50. The compound of any one of embodiments 39-41, 43, 45, and 46 wherein o is 1, 2, 3, or 4.

51. The compound of any one of embodiments 39-41 and 44-46, wherein r is 1, 2, 3, 4, or 5.

52. The compound of any one of embodiments 39-51, wherein R⁶ is deuterium, C₁-C₆ alkyl, —Si(C₁-C₆ alkyl)₃, phenyl, or —C(O)C₁-C₆ alkyl.

53. The compound of any one of embodiments 39-52, wherein R⁶ is CF₃.

54. The compound of any one of embodiments 39-51, wherein two R⁶s on a single carbon atom together with the carbon atom to which they are attached combine to form C₃-C₆ cycloalkyl.

55. The compound of any one of embodiments 39-51, wherein two R⁶s on different carbon atoms together with the carbon atoms to which they are attached combine to form a C₃-C₈ cycloalkyl, or 3-7 membered heterocyclyl.

56. The compound of embodiment 1, wherein the compound is of Formula (IA-5), (IB-5) or (IC-5):

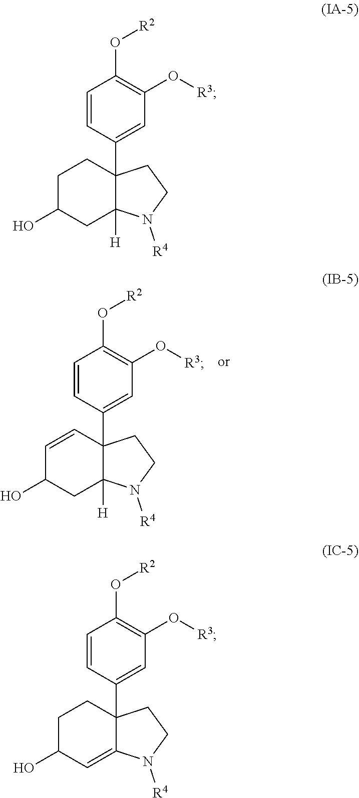

or a pharmaceutically acceptable salt thereof.

57. The compound of embodiment 56, wherein the compound is of Formula (IA-5a), (IA-5b), (IB-5a), (IB-5b), (IC-5a), or (IC-5b):

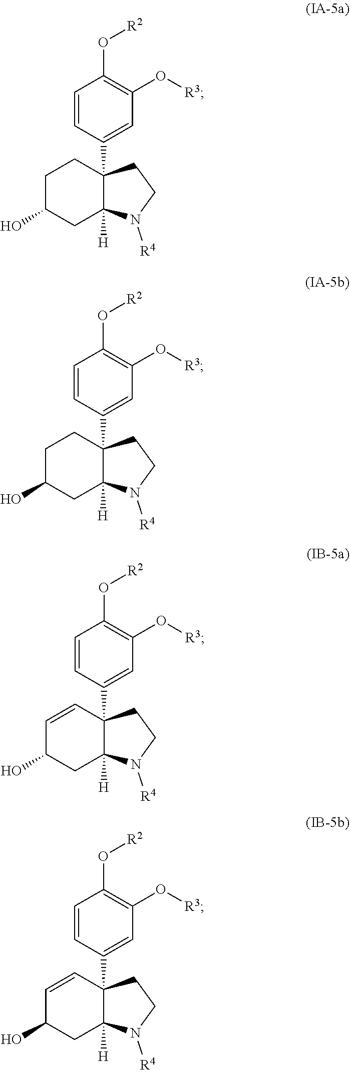

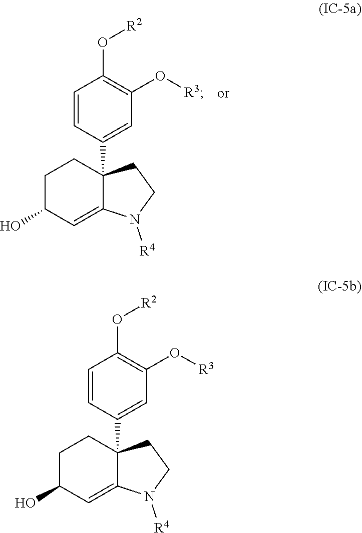

or a pharmaceutically acceptable salt thereof.

58. The compound of embodiment 57, wherein the compound is of Formula (IA-5a), (IA-5b), (IB-5a), (IB-5b), (IC-5a), or (IC-5b):

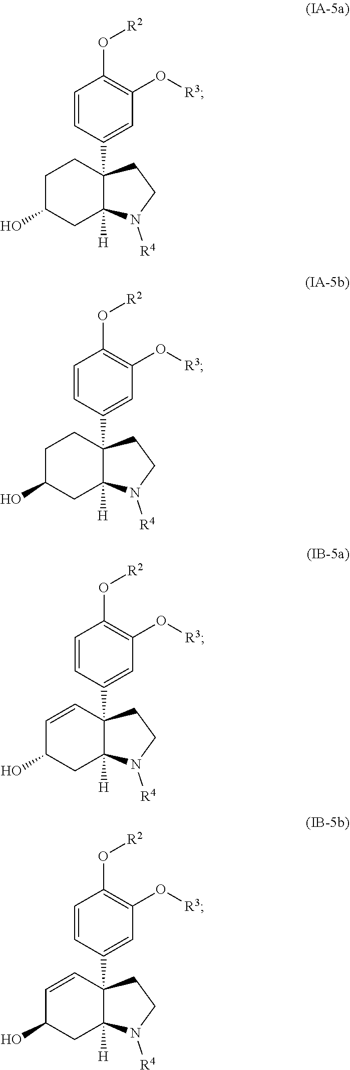

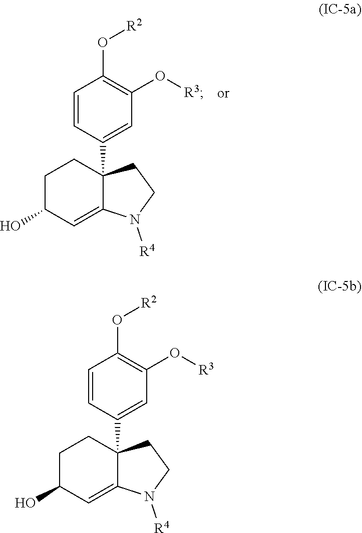

or a pharmaceutically acceptable salt thereof, wherein the compounds of Formula (IA-5a), (IA-5b), (IB-5a), (IB-5b), (IC-5a) and (IC-5b) have the absolute stereochemistry shown.

59. The compound of embodiment 58, wherein the compound is of Formula (IA-5), Formula (IA-5a) or Formula (IA-5b).

60. The compound of embodiment 58, wherein the compound is of Formula (IB-5), Formula (IB-5a) or Formula (IB-5b).

61. The compound of embodiment 58, wherein the compound is of Formula (IC-5), Formula (IC-5a) or Formula (IC-5b).

62. The compound of embodiment 1, wherein the compound is of Formula (IA-6), (IB-6) or (IC-6):

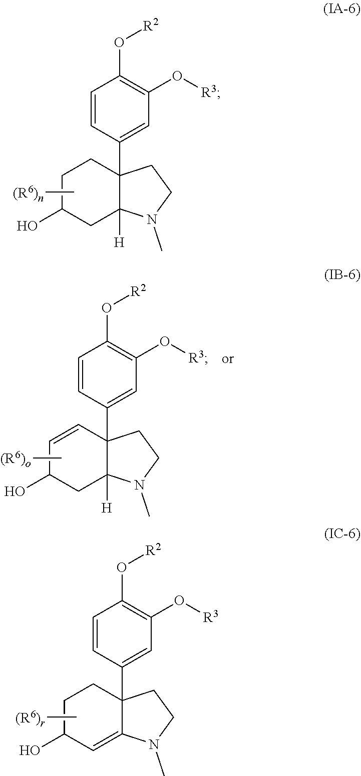

or a pharmaceutically acceptable salt thereof.

63. The compound of embodiment 62, wherein the compound is of Formula (IA-6a), (IA-6b), (IB-6a), (IB-6b), (IC-6a) or (IC-6b):

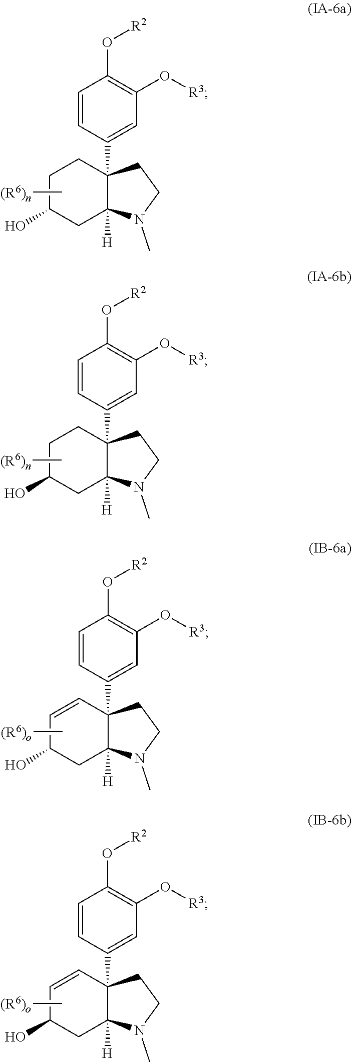

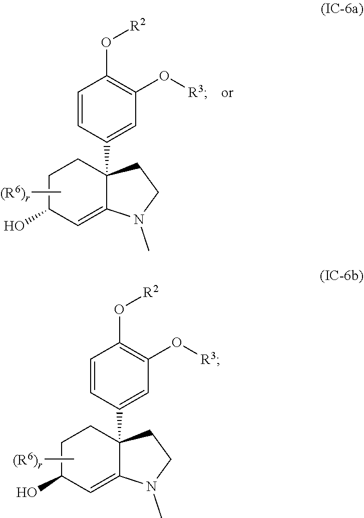

or a pharmaceutically acceptable salt thereof.

64. The compound of embodiment 63, wherein the compound is of Formula (IA-6a), (IA-6b), (IB-6a), (IB-6b), (IC-6a) or (IC-6b):

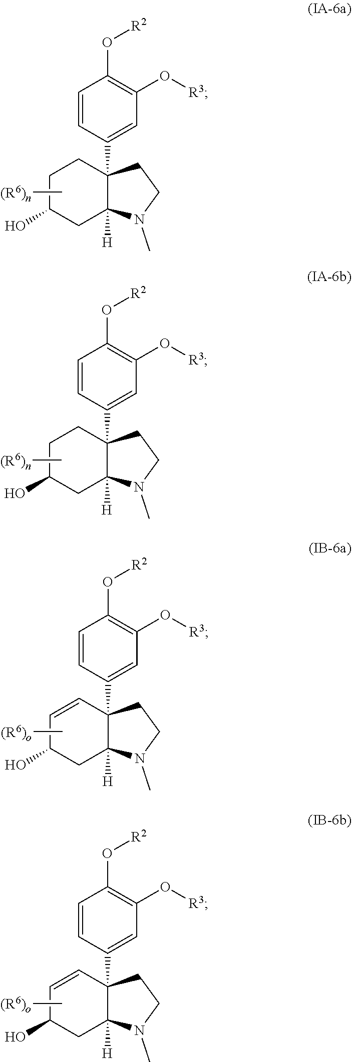

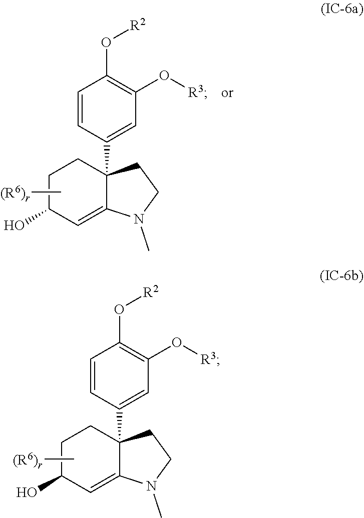

or a pharmaceutically acceptable salt thereof, wherein the compounds of Formula (IA-6a), (IA-6b), (IB-6a), (IB-6b), (IC-6a) and (IC-6b) have the absolute stereochemistry shown.

65. The compound of embodiment 64, wherein the compound is of Formula (IA-6), Formula (IA-6a) or Formula (IA-6b).

66. The compound of embodiment 64, wherein the compound is of Formula (IB-6), Formula (IB-6a) or Formula (IB-6b).

67. The compound of embodiment 64, wherein the compound is of Formula (IC-6), Formula (IC-6a) or Formula (IC-6b).

68. The compound of any one of embodiments 62-65, wherein n is 1, 2, 3, 4, 5, or 6.

69. The compound of any one of embodiments 62-64 and 66, wherein o is 1, 2, 3, or 4.

70. The compound of any one of embodiments 62-65 and 67, wherein r is 1, 2, 3, 4, or 5.

71. The compound of any one of embodiments 62-70, wherein R⁶ is deuterium, C₁-C₆ alkyl, —Si(C₁-C₆ alkyl)₃, phenyl, or —C(O)C₁-C₆ alkyl.

72. The compound of any one of embodiments 62-70, wherein R⁶ is CF₃.

73. The compound of any one of embodiments 62-70, wherein two R⁶s on a single carbon atom together with the carbon atom to which they are attached combine to form C₃-C₆ cycloalkyl.

74. The compound of any one of embodiments 62-70, wherein two R⁶s on different carbon atoms together with the carbon atoms to which they are attached combine to form a C₃-C₈ cycloalkyl, or 3-7 membered heterocyclyl.

75. The compound of embodiment 1, wherein the compound is of Formula (IA-7), (IB-7) or (IC-7):

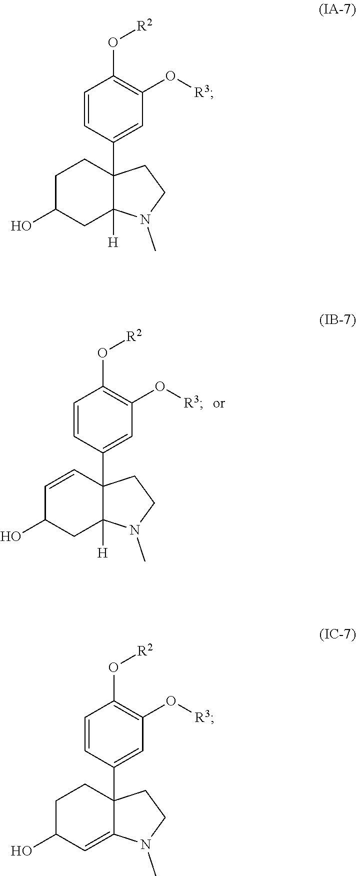

or a pharmaceutically acceptable salt thereof.

76. The compound of embodiment 75, wherein the compound is of Formula (IA-7a), (IA-7b), (IB-7a), (IB-7b), (IC-7a) or (IC-7b):

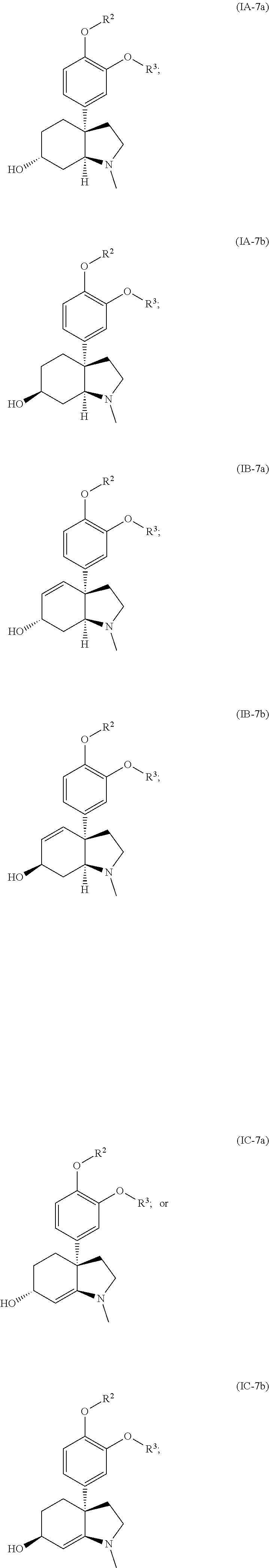

or a pharmaceutically acceptable salt thereof.

77. The compound of embodiment 76, wherein the compound is of Formula (IA-7a), (IA-7b), (IB-7a), (IB-7b), (IC-7a) or (IC-7b):

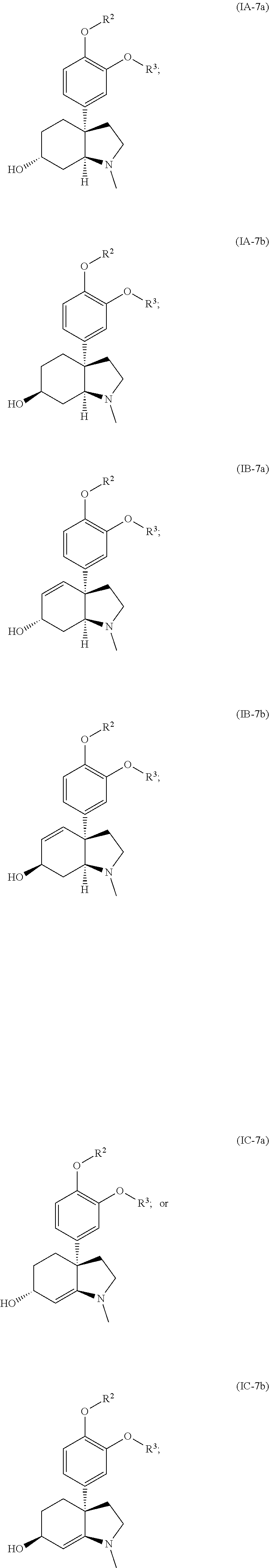

or a pharmaceutically acceptable salt thereof, wherein the compounds of Formula (IA-7a), (IA-7b), (IB-7a), (IB-7b), (IC-7a) and (IC-7b) have the absolute stereochemistry shown.

78. The compound of embodiment 77, wherein the compound is of Formula (IA-7), Formula (IA-7a) or Formula (IA-7b).

79. The compound of embodiment 77, wherein the compound is of Formula (IB-7), Formula (IB-7a) or Formula (IB-7b).

80. The compound of embodiment 77, wherein the compound is of Formula (IC-7), Formula (IC-7a) or Formula (IC-7b).

81. The compound of any one of the preceding embodiments, wherein each of R² and R³ is individually H, C₃-C₆ cycloalkyl, C₁-C₆ alkyl optionally substituted by fluoro, deuterium, C₃-C₆ cycloalkyl, C₂-C₆ alkenyl, C₂-C₆ alkynyl or —OC₁-C₆ alkyl,

82. The compound of any one of the preceding embodiments, wherein R² and R³ together with the atoms to which they are attached combine to form 5 to 7-membered heterocyclyl or 5 to 7-membered heteroaryl, wherein each hydrogen atom in 5 to 7-membered heterocyclyl and 5 to 7-membered heteroaryl is optionally substituted by halo or OR^{a}.

83. The compound of embodiment 1, wherein the compound is of Formula (IA-8), (IB-8) or (IC-8):

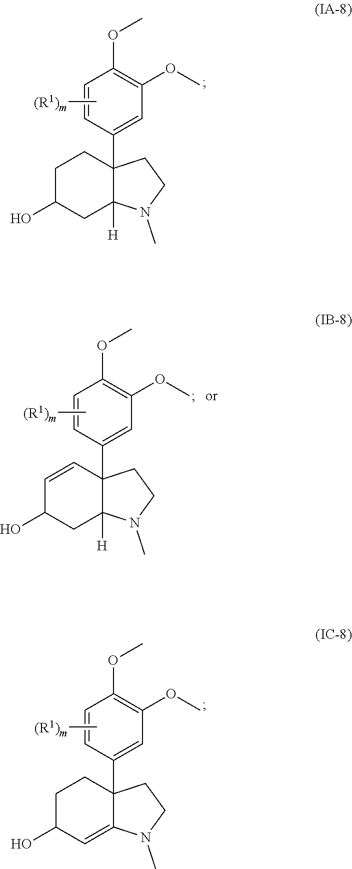

or a pharmaceutically acceptable salt thereof.

84. The compound of embodiment 83, wherein the compound is of Formula (IA-8a), (IA-8b), (IB-8a), (IB-8b), (IC-8a) or (IC-8b):

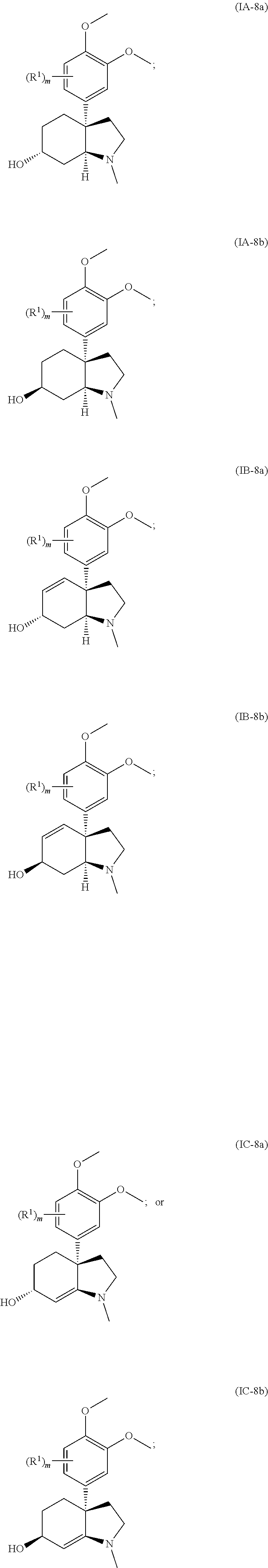

or a pharmaceutically acceptable salt thereof.

85. The compound of embodiment 84, wherein the compound is of Formula (IA-8a), (IA-8b), (IB-8a), (IB-8b), (IC-8a) or (IC-8b):

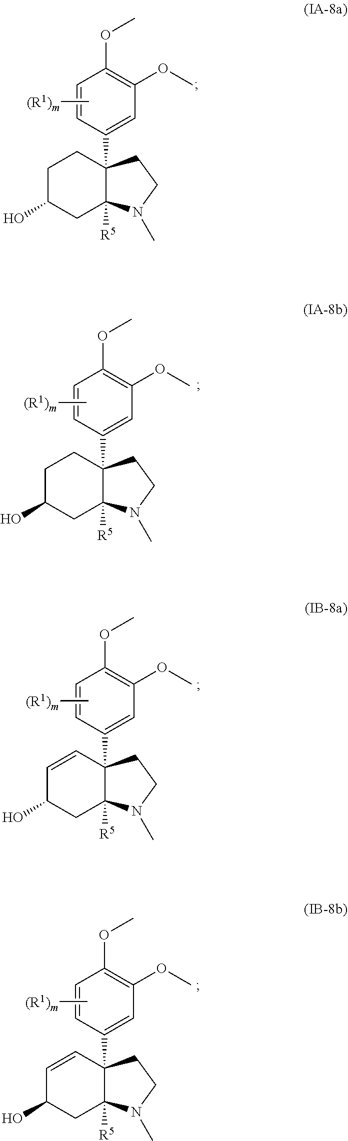

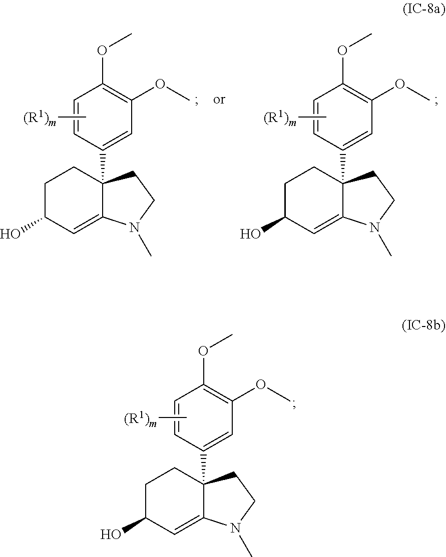

or a pharmaceutically acceptable salt thereof, wherein the compounds of Formula (IA-8a), (IA-8b), (IB-8a), (IB-8b), (IC-8a) and (IC-8b) have the absolute stereochemistry shown.

86. The compound of embodiment 85, wherein the compound is of Formula (IA-8), Formula (IA-8a) or Formula (IA-8b).

87. The compound of embodiment 85, wherein the compound is of Formula (IB-8), Formula (IB-8a) or Formula (IB-8b).

88. The compound of embodiment 85, wherein the compound is of Formula (IC-8), Formula (IC-8a) or Formula (IC-8b).

89. The compound of any one of embodiments 83-88, wherein m is 1, 2, or 3.

90. The compound of any one of embodiments 82-88, wherein m is 1.

91. The compound of any one of embodiments 82-90, wherein R¹ is halo, alkenyl, cycloalkyl, cyano, or —C(O)NR^{a}R^{b}.

92. The compound of any one of embodiments 82-90, wherein R¹ is fluoro, chloro, bromo, iodo, vinyl, cyclopropyl, cyano, or —C(O)NH₂.

93. The compound of embodiment 1, wherein the compound is of Formula (IA-9), (IB-9) or (IC-9):

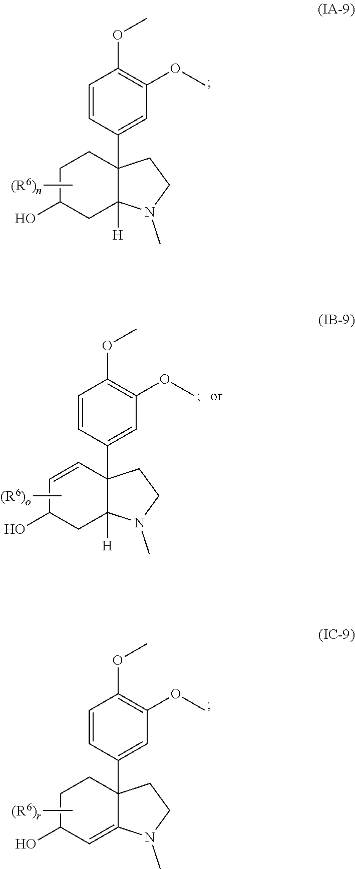

or a pharmaceutically acceptable salt thereof.

94. The compound of embodiment 93, the compound is of Formula (IA-9a), (IA-9b), (IB-9a), (IB-9b), (IC-9a) or (IC-9b):

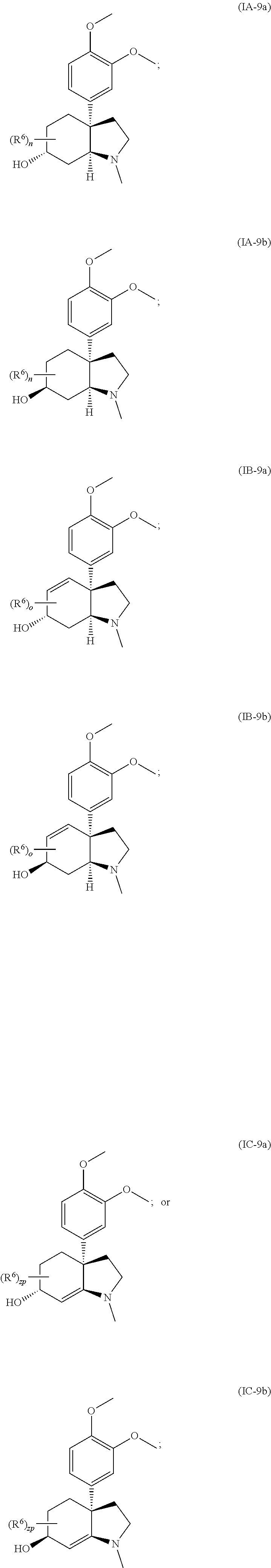

or a pharmaceutically acceptable salt thereof.

95. The compound of embodiment 94, the compound is of Formula (IA-9a), (IA-9b), (IB-9a), (IB-9b), (IC-9a) or (IC-9b):

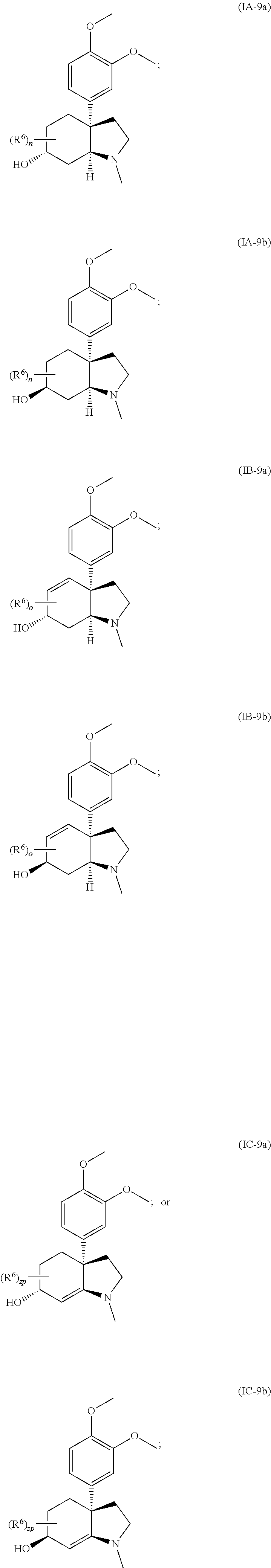

or a pharmaceutically acceptable salt thereof, wherein the compounds of Formula (IA-9a), (IA-9b), (IB-9a), (IB-9b), (IC-9a), and (IC-9b) have the absolute stereochemistry shown.

96. The compound of embodiment 95, wherein the compound is of Formula (IA-9), Formula (IA-9a) or Formula (IA-9b).

97. The compound of embodiment 95, wherein the compound is of Formula (IB-9), Formula (IB-9a) or Formula (IB-9b).

98. The compound of embodiment 99, wherein the compound is of Formula (IC-9), Formula (IC-9a) or Formula (IC-9b).

99. The compound of any one of embodiments 93-96, wherein n is 1, 2, 3, 4, 5, or 6.

100. The compound of any one of embodiments 93-95 and 97, wherein o is 1, 2, 3, or 4.

101. The compound of any one of embodiments 93-95 and 98, wherein r is 1, 2, 3, 4, or 5.

102. The compound of any one of embodiments 93-101, wherein R⁶ is deuterium, C₁-C₆ alkyl, —Si(C₁-C₆ alkyl)₃, phenyl, or —C(O)C₁-C₆ alkyl.

103. The compound of any one of embodiments 93-101, wherein R⁶ is CF₃.

104. The compound of any one of embodiments 93-101, wherein two R⁶s on a single carbon atom together with the carbon atom to which they are attached combine to form C₃-C₆ cycloalkyl.

105. The compound of any one of embodiments 93-101, wherein two R⁶s on different carbon atoms together with the carbon atoms to which they are attached combine to form a C₃-C₈ cycloalkyl, or 3-7 membered heterocyclyl.

106. The compound of embodiment 1, wherein the compound is of Formula (IA-10), (IB-10), or (IC-10):

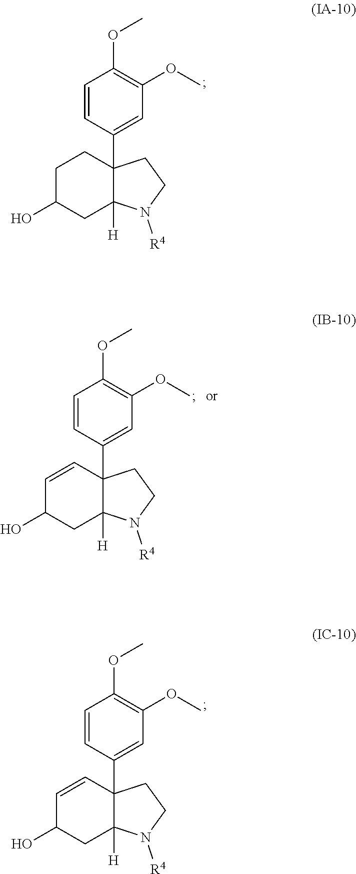

or a pharmaceutically acceptable salt thereof.

107. The compound of embodiment 106, wherein the compound is of Formula (IA-10a), (IA-10b), (IB-10a), (IB-10b), (IC-10a) or (IC-10b):

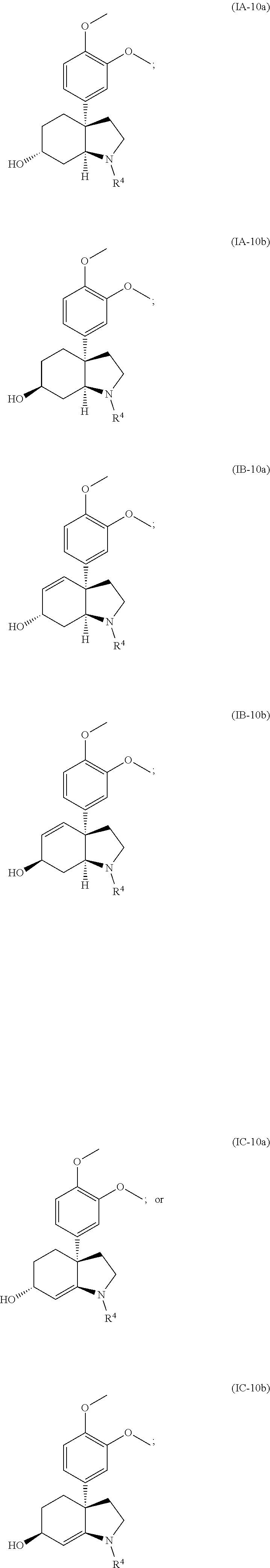

or a pharmaceutically acceptable salt thereof.

108. The compound of embodiment 107, wherein the compound is of Formula (IA-10a), (IA-10b), (IB-10a), (IB-10b), (IC-10a) or (IC-10b):

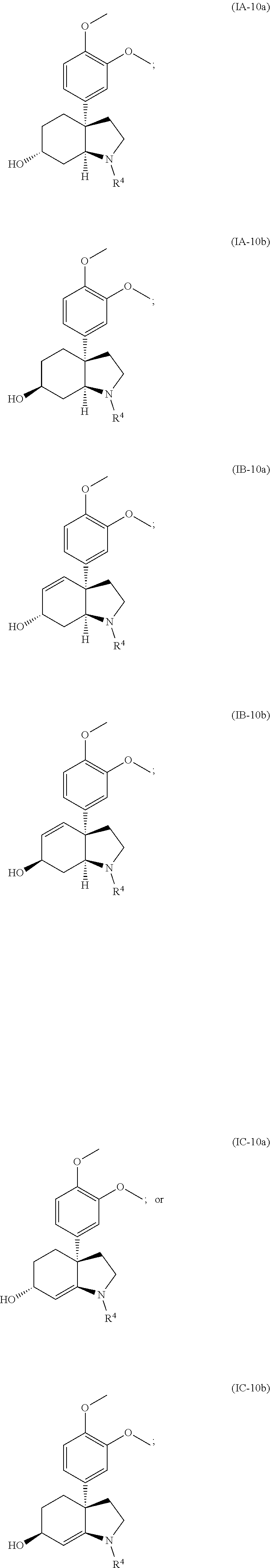

or a pharmaceutically acceptable salt thereof, wherein the compounds of Formula (IA-10a), (IA-10b), (IB-10a), (IB-10b), (IC-10a) or (IC-10b) have the absolute stereochemistry shown.

109. The compound of embodiment 108, wherein the compound is of Formula (IA-10), Formula (IA-10) or Formula (IA-10).

110. The compound of embodiment 108, wherein the compound is of Formula (IB-10), Formula (IB-10) or Formula (IB-10).

111. The compound of embodiment 108, wherein the compound is of Formula (IC-10), Formula (IC-10) or Formula (IC-10).

112. The compound of any one of embodiments 106-111, wherein R⁴ is H or C₁-C₆ alkyl optionally substituted by deuterium, phenyl optionally substituted by —OC₁-C₆ alkyl, or C₃-C₆ cycloalkyl.

113. The compound of any one of embodiments 106-111, wherein R⁴ is methyl or CD₃.

114. The compound of embodiment 1, selected from:

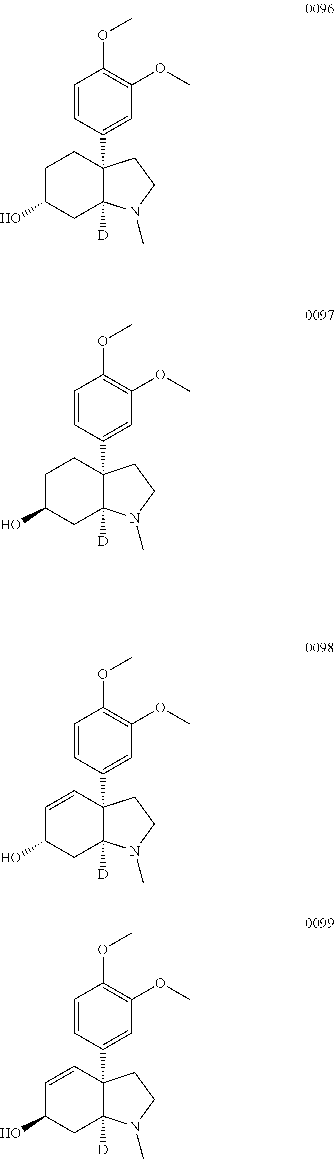 [SMILES: [2H][C@]12C[C@H](O)CC[C@@]1(c1ccc(OC)c(OC)c1)CCN2C ; [2H][C@]12C[C@@H](O)CC[C@@]1(c1ccc(OC)c(OC)c1)CCN2C ; [2H][C@]12C[C@H](O)C=C[C@@]1(c1ccc(OC)c(OC)c1)CCN2C ; [2H][C@]12C[C@@H](O)C=C[C@@]1(c1ccc(OC)c(OC)c1)CCN2C]

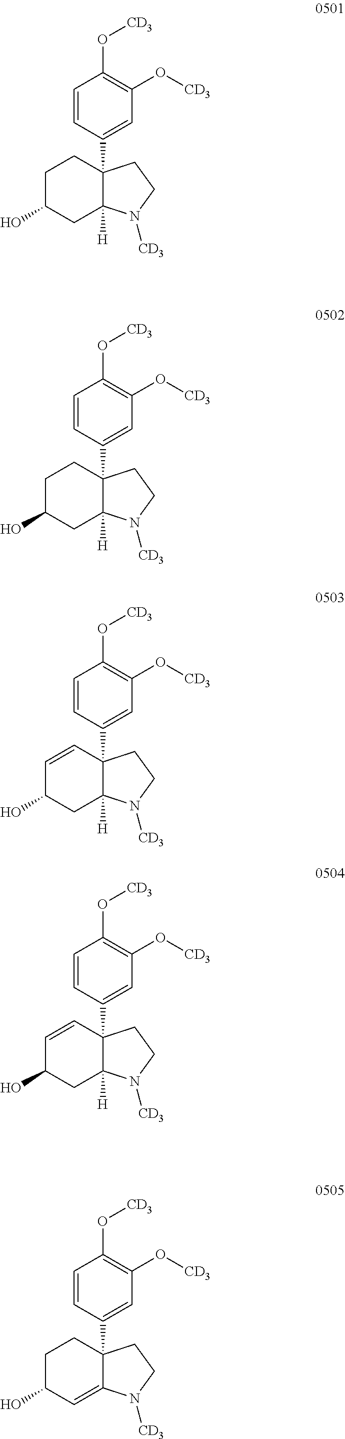 [SMILES: [2H]C([2H])([2H])Oc1ccc([C@@]23CC[C@@H](O)C[C@@H]2N(C([2H])([2H])[2H])CC3)cc1OC([2H])([2H])[2H] ; [2H]C([2H])([2H])Oc1ccc([C@@]23CC[C@H](O)C[C@@H]2N(C([2H])([2H])[2H])CC3)cc1OC([2H])([2H])[2H] ; [2H]C([2H])([2H])Oc1ccc([C@@]23C=C[C@@H](O)C[C@@H]2N(C([2H])([2H])[2H])CC3)cc1OC([2H])([2H])[2H] ; [2H]C([2H])([2H])Oc1ccc([C@@]23C=C[C@H](O)C[C@@H]2N(C([2H])([2H])[2H])CC3)cc1OC([2H])([2H])[2H] ; [2H]C([2H])([2H])Oc1ccc([C@@]23CC[C@@H](O)C=C2N(C([2H])([2H])[2H])CC3)cc1OC([2H])([2H])[2H]]

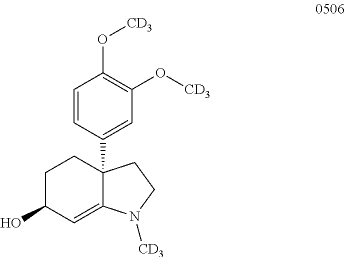 [SMILES: [2H]C([2H])([2H])Oc1ccc([C@@]23CC[C@H](O)C=C2N(C([2H])([2H])[2H])CC3)cc1OC([2H])([2H])[2H]]

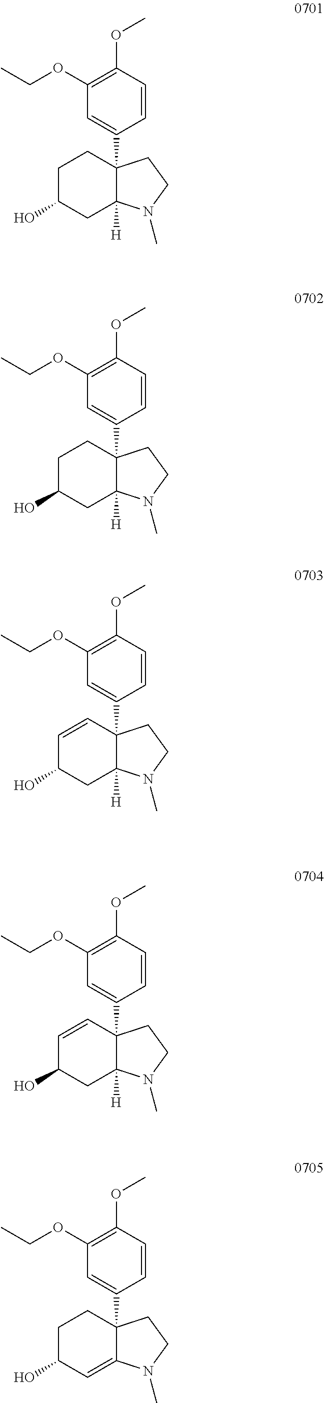 [SMILES: CCOc1cc([C@@]23CC[C@@H](O)C[C@@H]2N(C)CC3)ccc1OC ; CCOc1cc([C@@]23CC[C@H](O)C[C@@H]2N(C)CC3)ccc1OC ; CCOc1cc([C@@]23C=C[C@@H](O)C[C@@H]2N(C)CC3)ccc1OC ; CCOc1cc([C@@]23C=C[C@H](O)C[C@@H]2N(C)CC3)ccc1OC ; CCOc1cc([C@@]23CC[C@@H](O)C=C2N(C)CC3)ccc1OC]

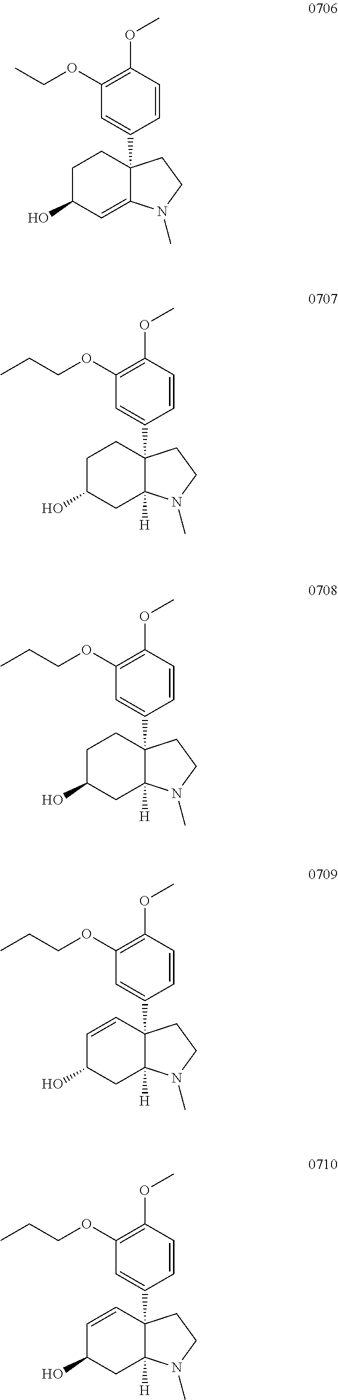 [SMILES: CCOc1cc([C@@]23CC[C@H](O)C=C2N(C)CC3)ccc1OC ; CCCOc1cc([C@@]23CC[C@@H](O)C[C@@H]2N(C)CC3)ccc1OC ; CCCOc1cc([C@@]23CC[C@H](O)C[C@@H]2N(C)CC3)ccc1OC ; CCCOc1cc([C@@]23C=C[C@@H](O)C[C@@H]2N(C)CC3)ccc1OC ; CCCOc1cc([C@@]23C=C[C@H](O)C[C@@H]2N(C)CC3)ccc1OC]

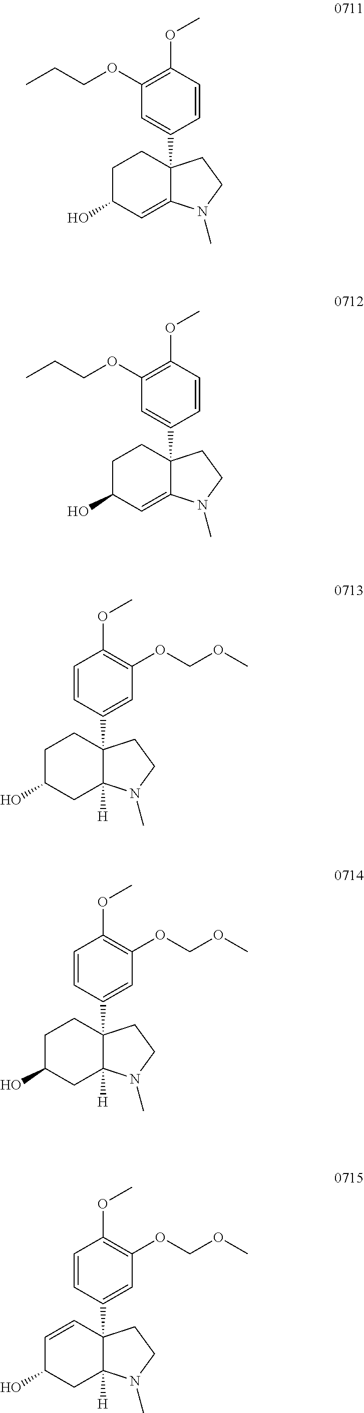 [SMILES: CCCOc1cc([C@@]23CC[C@@H](O)C=C2N(C)CC3)ccc1OC ; CCCOc1cc([C@@]23CC[C@H](O)C=C2N(C)CC3)ccc1OC ; COCOc1cc([C@@]23CC[C@@H](O)C[C@@H]2N(C)CC3)ccc1OC ; COCOc1cc([C@@]23CC[C@H](O)C[C@@H]2N(C)CC3)ccc1OC ; COCOc1cc([C@@]23C=C[C@@H](O)C[C@@H]2N(C)CC3)ccc1OC]

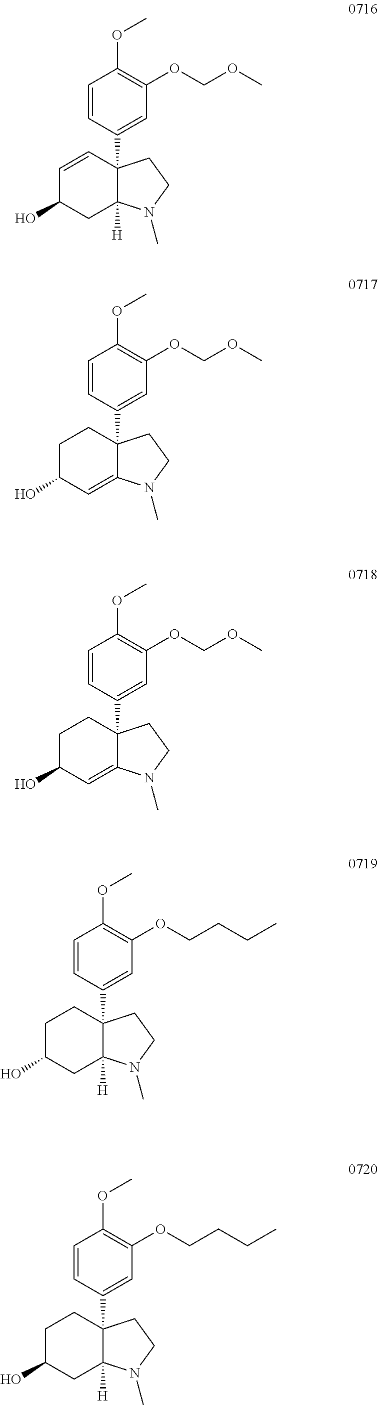 [SMILES: COCOc1cc([C@@]23C=C[C@H](O)C[C@@H]2N(C)CC3)ccc1OC ; COCOc1cc([C@@]23CC[C@@H](O)C=C2N(C)CC3)ccc1OC ; COCOc1cc([C@@]23CC[C@H](O)C=C2N(C)CC3)ccc1OC ; CCCCOc1cc([C@@]23CC[C@@H](O)C[C@@H]2N(C)CC3)ccc1OC ; CCCCOc1cc([C@@]23CC[C@H](O)C[C@@H]2N(C)CC3)ccc1OC]

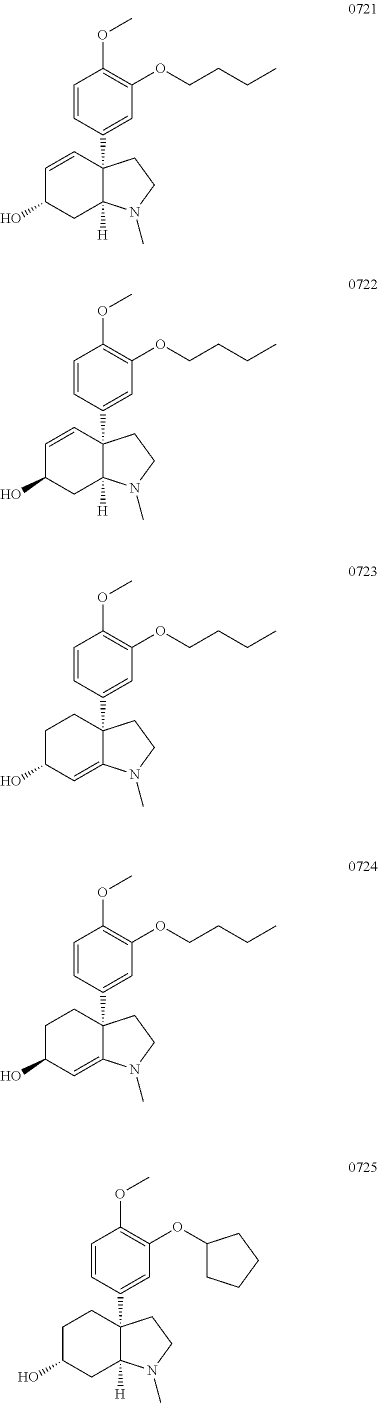 [SMILES: CCCCOc1cc([C@@]23C=C[C@@H](O)C[C@@H]2N(C)CC3)ccc1OC ; CCCCOc1cc([C@@]23C=C[C@H](O)C[C@@H]2N(C)CC3)ccc1OC ; CCCCOc1cc([C@@]23CC[C@@H](O)C=C2N(C)CC3)ccc1OC ; CCCCOc1cc([C@@]23CC[C@H](O)C=C2N(C)CC3)ccc1OC ; COc1ccc([C@@]23CC[C@@H](O)C[C@@H]2N(C)CC3)cc1OC1CCCC1]

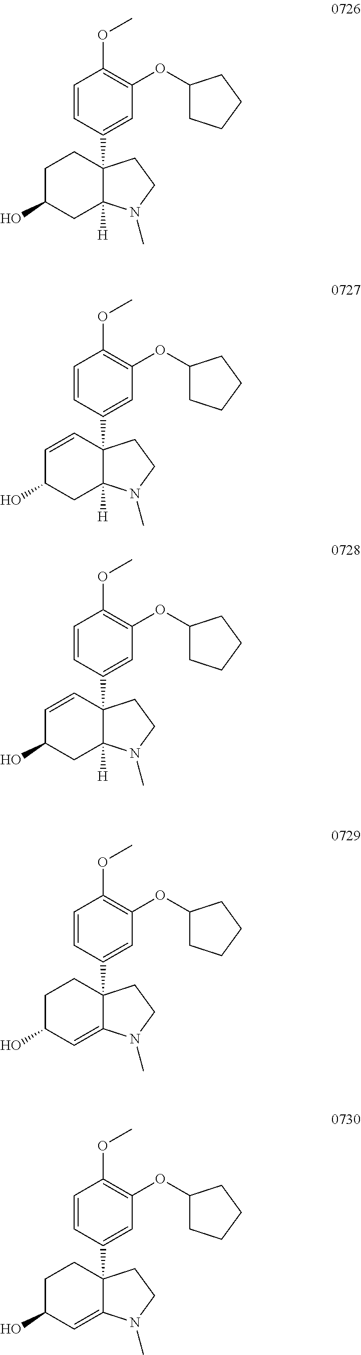

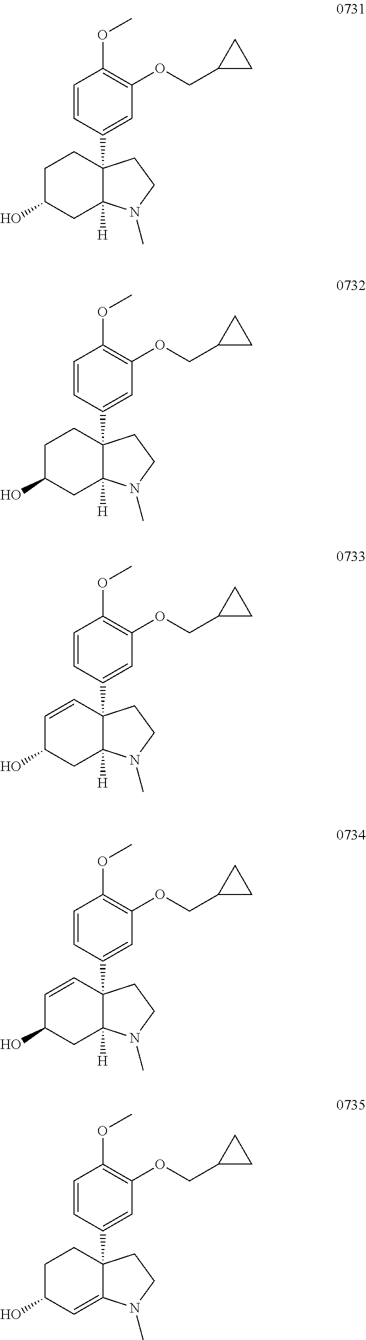

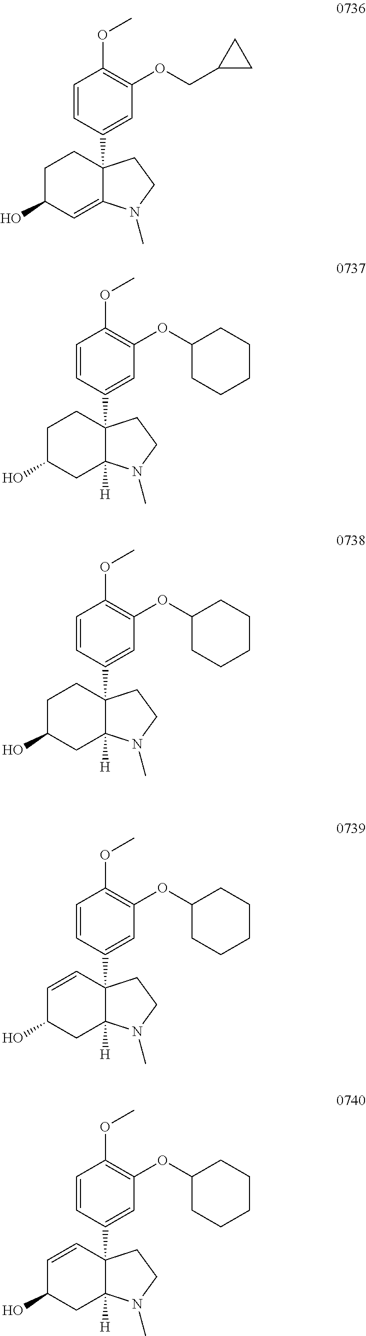

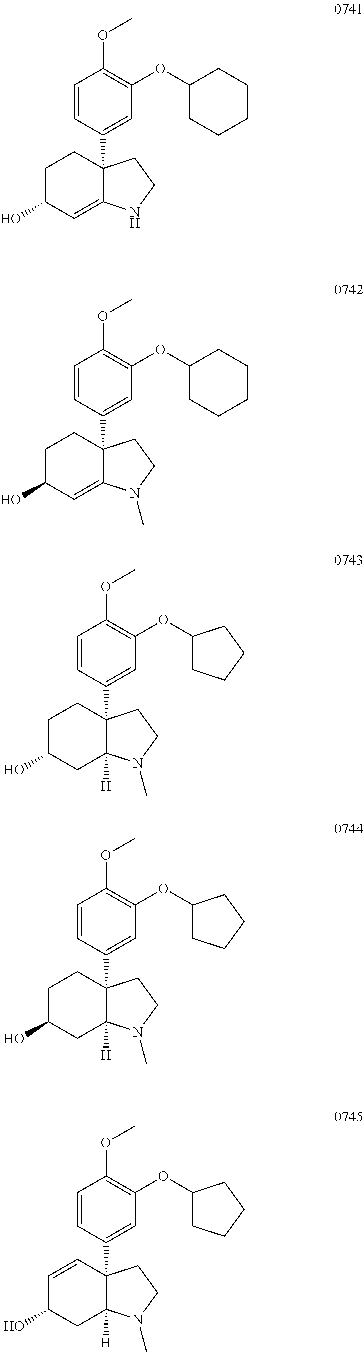

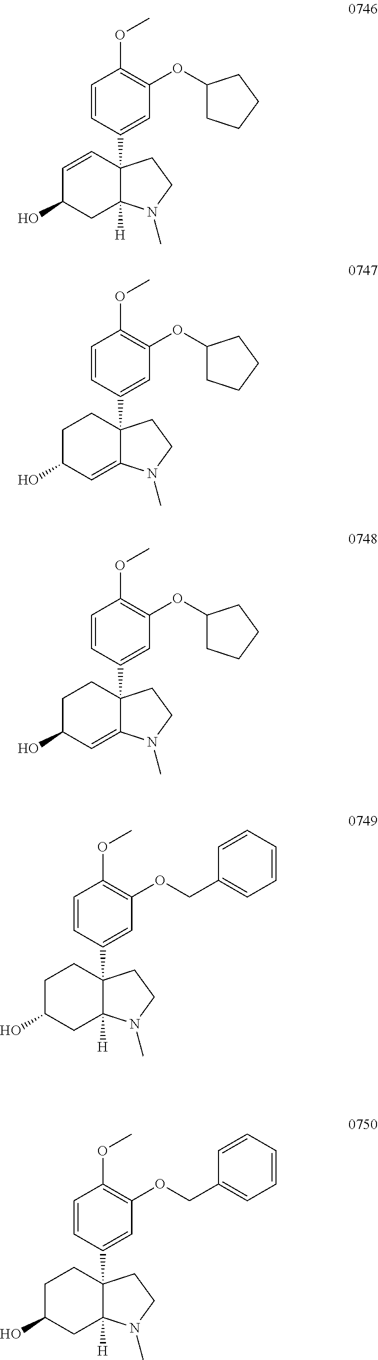

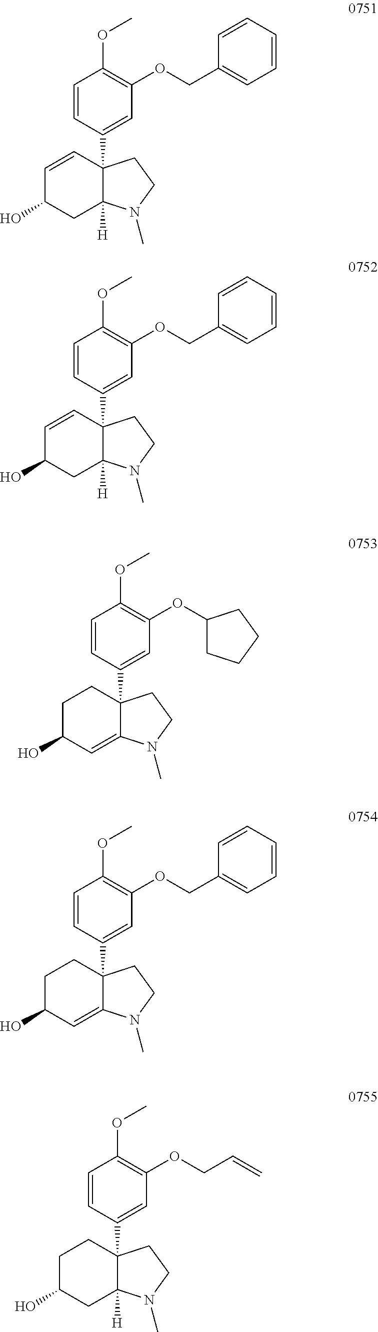

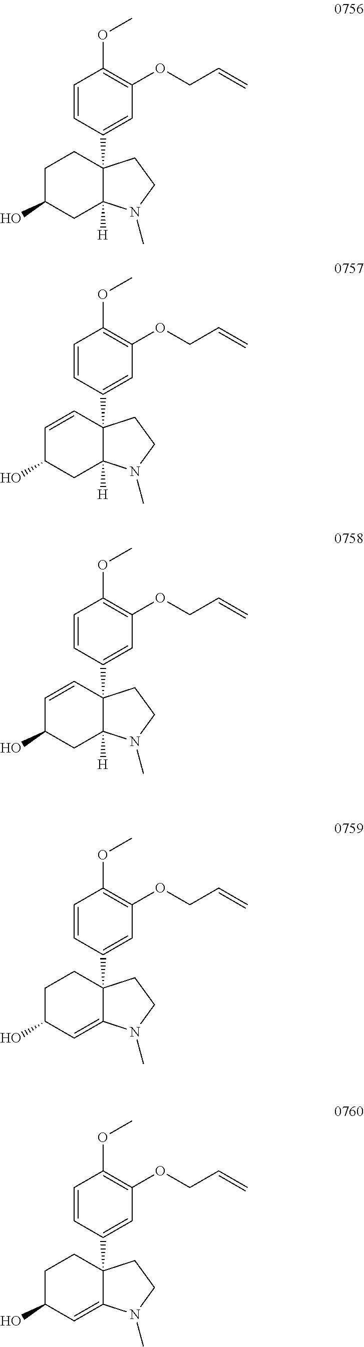

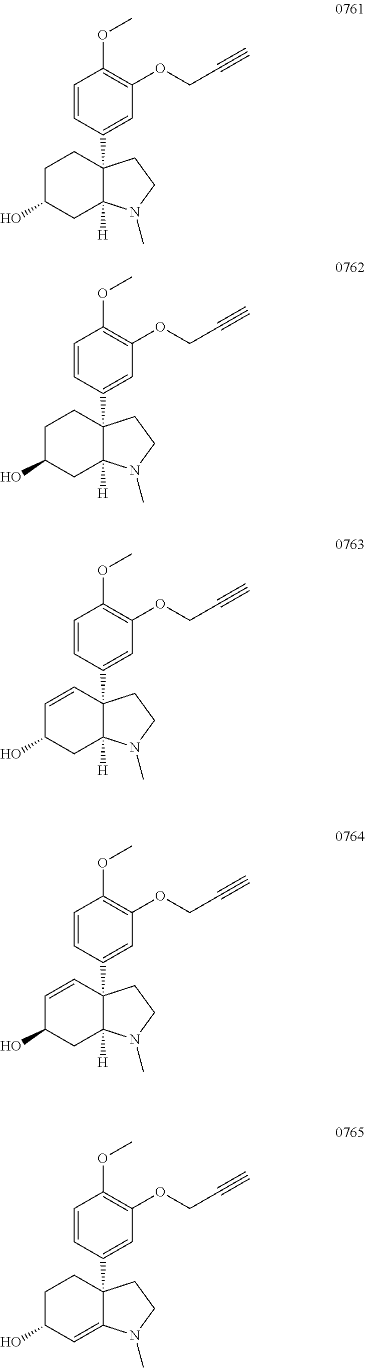

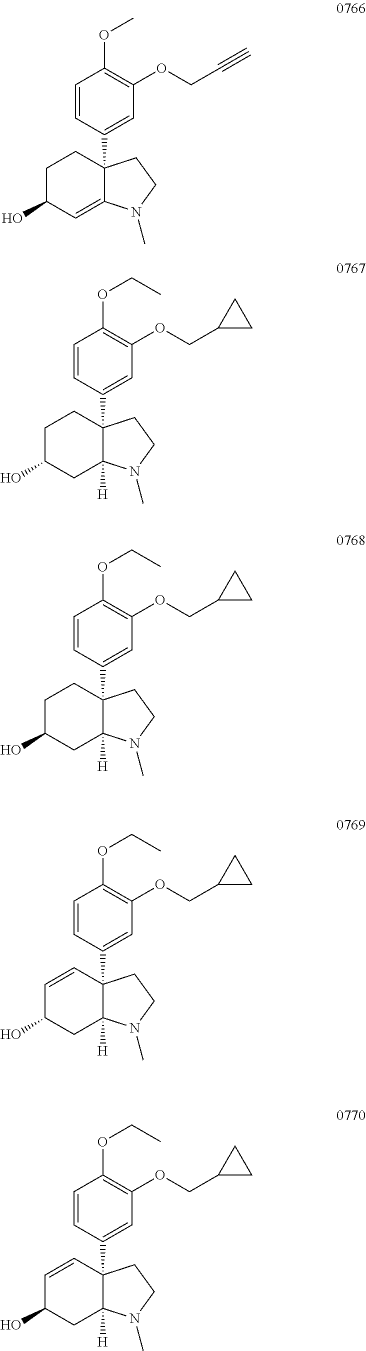

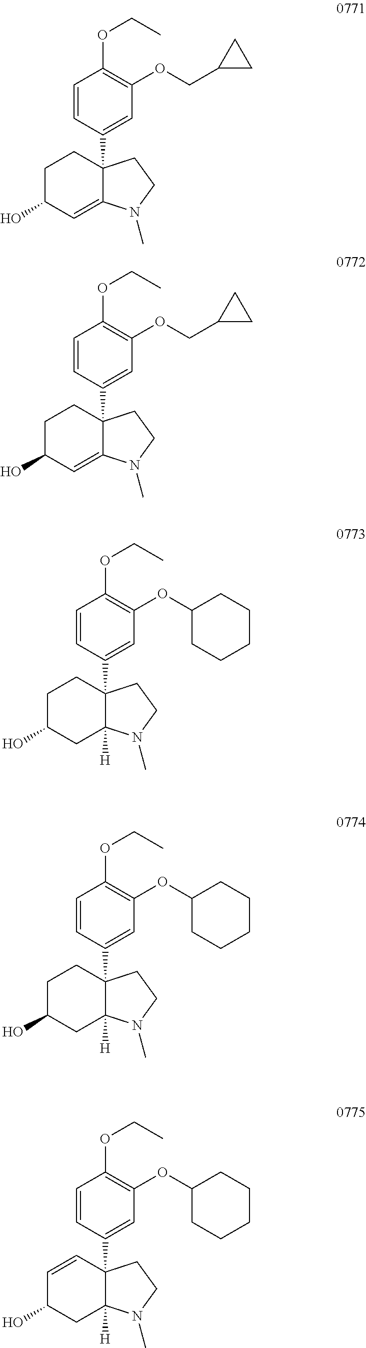

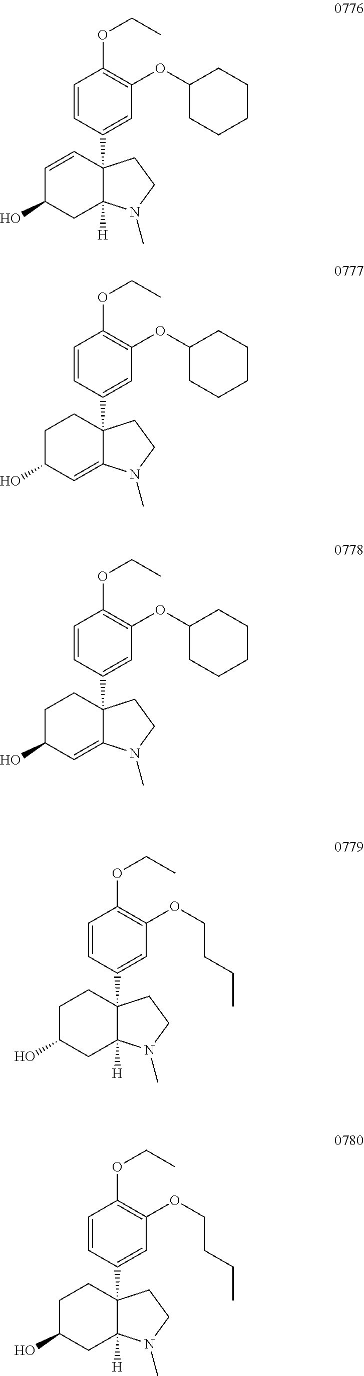

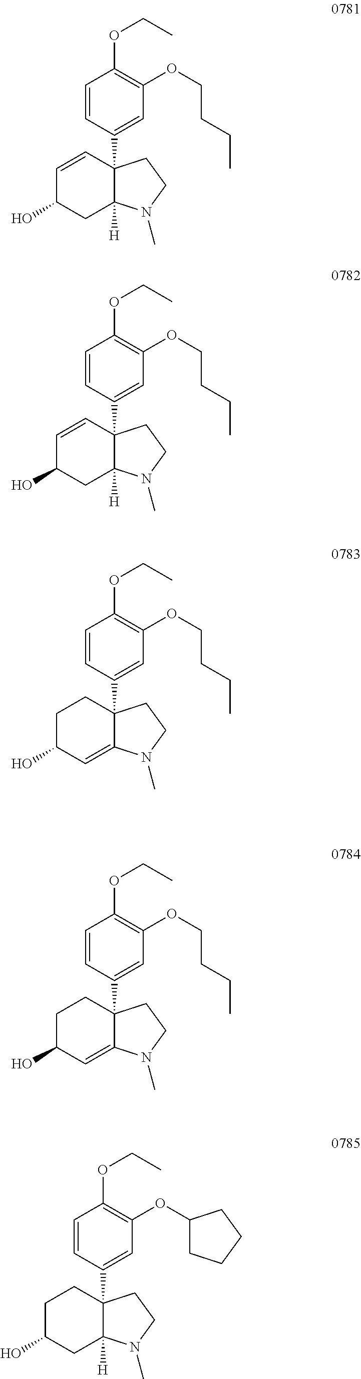

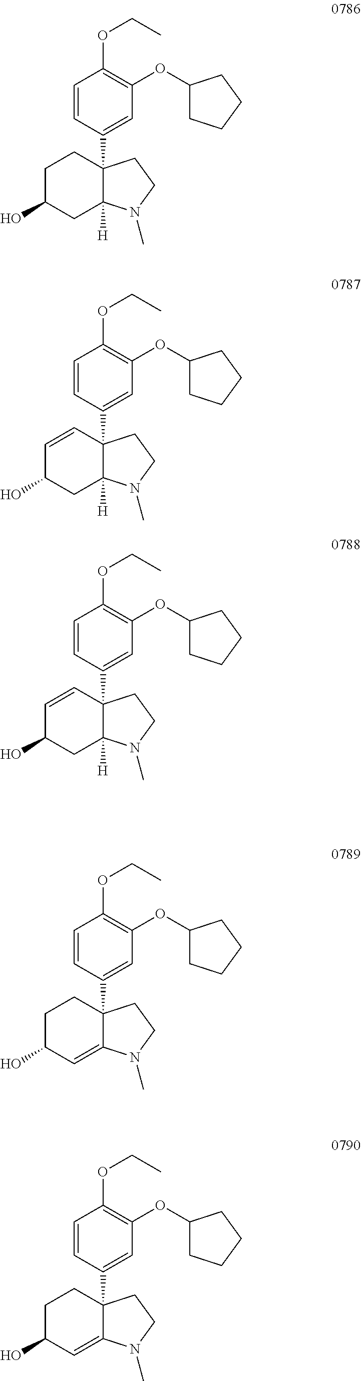

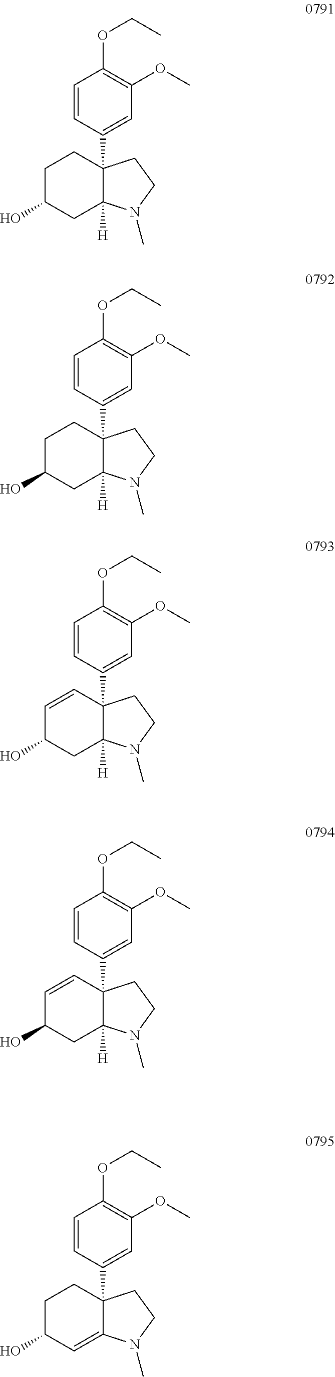

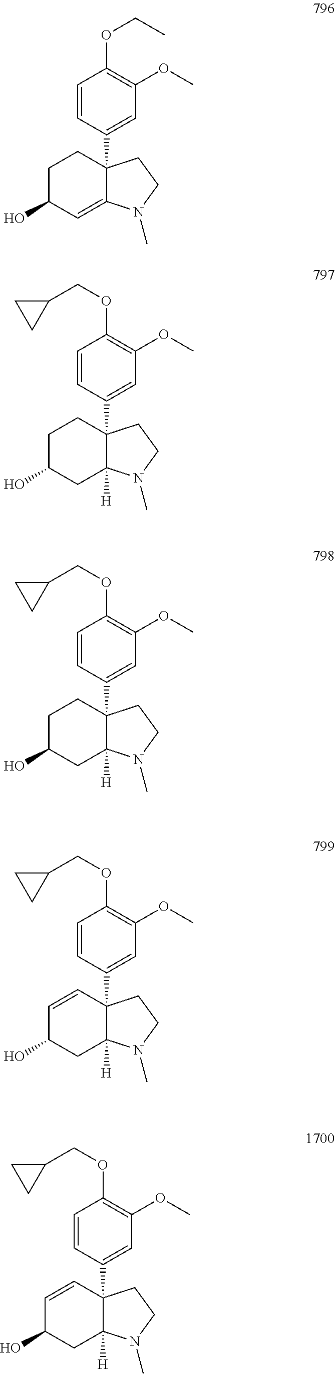 [SMILES: CCOc1ccc([C@@]23CC[C@H](O)C=C2N(C)CC3)cc1OC ; COc1cc([C@@]23CC[C@@H](O)C[C@@H]2N(C)CC3)ccc1OCC1CC1 ; COc1cc([C@@]23CC[C@H](O)C[C@@H]2N(C)CC3)ccc1OCC1CC1 ; COc1cc([C@@]23C=C[C@@H](O)C[C@@H]2N(C)CC3)ccc1OCC1CC1 ; COc1cc([C@@]23C=C[C@H](O)C[C@@H]2N(C)CC3)ccc1OCC1CC1]

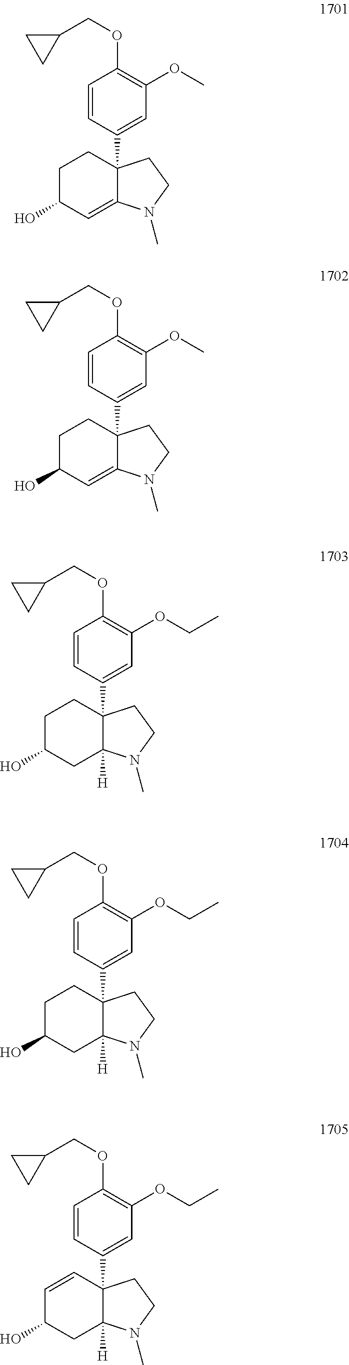 [SMILES: COc1cc([C@@]23CC[C@@H](O)C=C2N(C)CC3)ccc1OCC1CC1 ; COc1cc([C@@]23CC[C@H](O)C=C2N(C)CC3)ccc1OCC1CC1 ; CCOc1cc([C@@]23CC[C@@H](O)C[C@@H]2N(C)CC3)ccc1OCC1CC1 ; CCOc1cc([C@@]23CC[C@H](O)C[C@@H]2N(C)CC3)ccc1OCC1CC1 ; CCOc1cc([C@@]23C=C[C@@H](O)C[C@@H]2N(C)CC3)ccc1OCC1CC1]

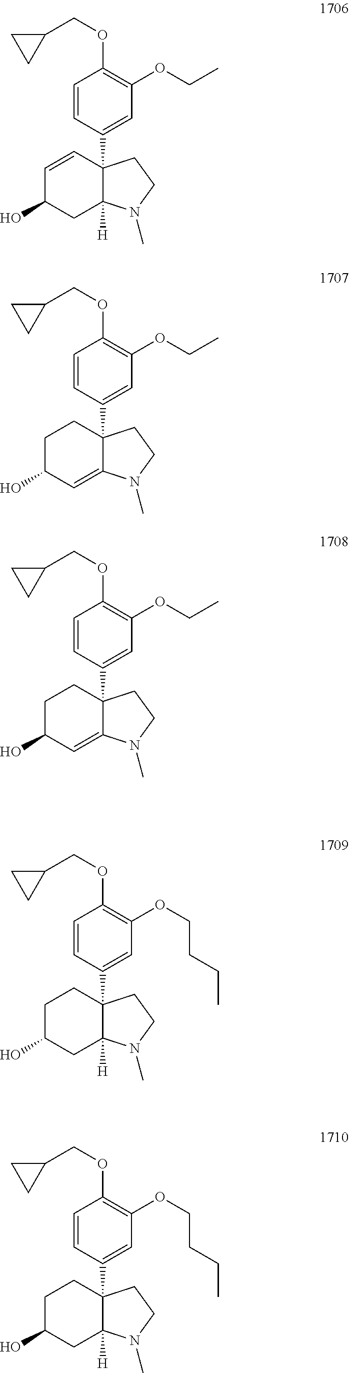 [SMILES: CCOc1cc([C@@]23C=C[C@H](O)C[C@@H]2N(C)CC3)ccc1OCC1CC1 ; CCOc1cc([C@@]23CC[C@@H](O)C=C2N(C)CC3)ccc1OCC1CC1 ; CCOc1cc([C@@]23CC[C@H](O)C=C2N(C)CC3)ccc1OCC1CC1 ; CCCCOc1cc([C@@]23CC[C@@H](O)C[C@@H]2N(C)CC3)ccc1OCC1CC1 ; CCCCOc1cc([C@@]23CC[C@H](O)C[C@@H]2N(C)CC3)ccc1OCC1CC1]

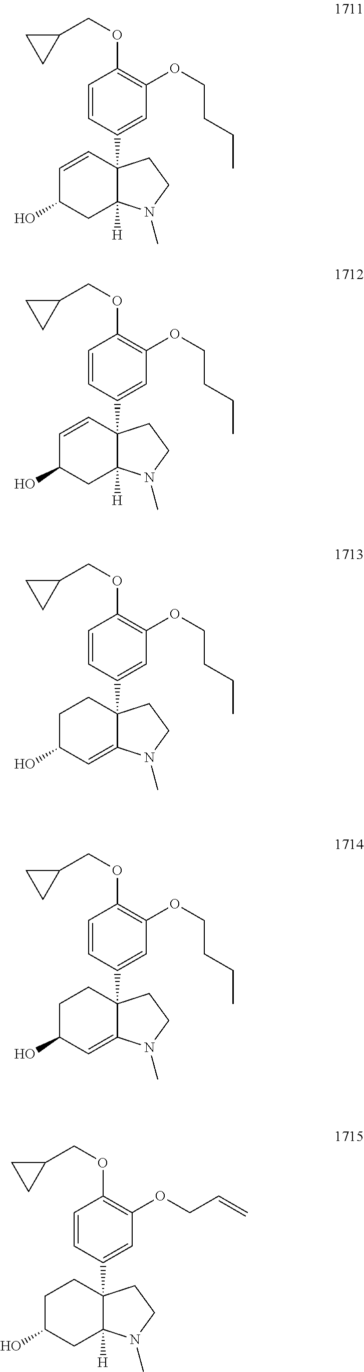 [SMILES: CCCCOc1cc([C@@]23C=C[C@@H](O)C[C@@H]2N(C)CC3)ccc1OCC1CC1 ; CCCCOc1cc([C@@]23C=C[C@H](O)C[C@@H]2N(C)CC3)ccc1OCC1CC1 ; CCCCOc1cc([C@@]23CC[C@@H](O)C=C2N(C)CC3)ccc1OCC1CC1 ; CCCCOc1cc([C@@]23CC[C@H](O)C=C2N(C)CC3)ccc1OCC1CC1 ; C=CCOc1cc([C@@]23CC[C@@H](O)C[C@@H]2N(C)CC3)ccc1OCC1CC1]

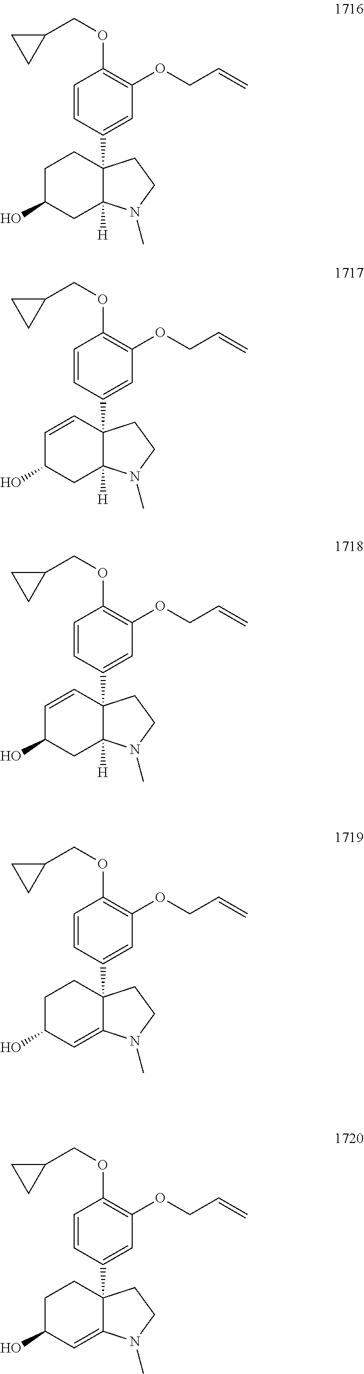 [SMILES: C=CCOc1cc([C@@]23CC[C@H](O)C[C@@H]2N(C)CC3)ccc1OCC1CC1 ; C=CCOc1cc([C@@]23C=C[C@@H](O)C[C@@H]2N(C)CC3)ccc1OCC1CC1 ; C=CCOc1cc([C@@]23C=C[C@H](O)C[C@@H]2N(C)CC3)ccc1OCC1CC1 ; C=CCOc1cc([C@@]23CC[C@@H](O)C=C2N(C)CC3)ccc1OCC1CC1 ; C=CCOc1cc([C@@]23CC[C@H](O)C=C2N(C)CC3)ccc1OCC1CC1]

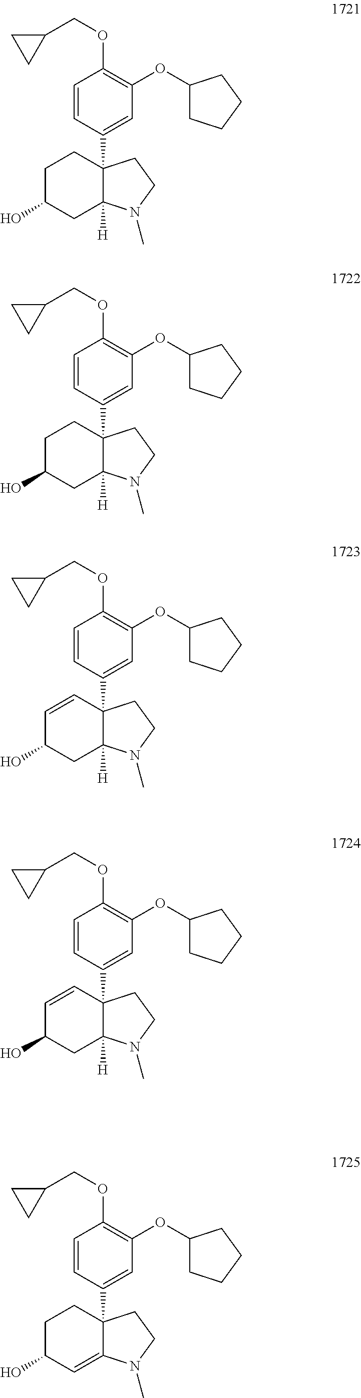 [SMILES: CN1CC[C@]2(c3ccc(OCC4CC4)c(OC4CCCC4)c3)CC[C@@H](O)C[C@H]12 ; CN1CC[C@]2(c3ccc(OCC4CC4)c(OC4CCCC4)c3)CC[C@H](O)C[C@H]12 ; CN1CC[C@]2(c3ccc(OCC4CC4)c(OC4CCCC4)c3)C=C[C@@H](O)C[C@H]12 ; CN1CC[C@]2(c3ccc(OCC4CC4)c(OC4CCCC4)c3)C=C[C@H](O)C[C@H]12 ; CN1CC[C@]2(c3ccc(OCC4CC4)c(OC4CCCC4)c3)CC[C@@H](O)C=C12]

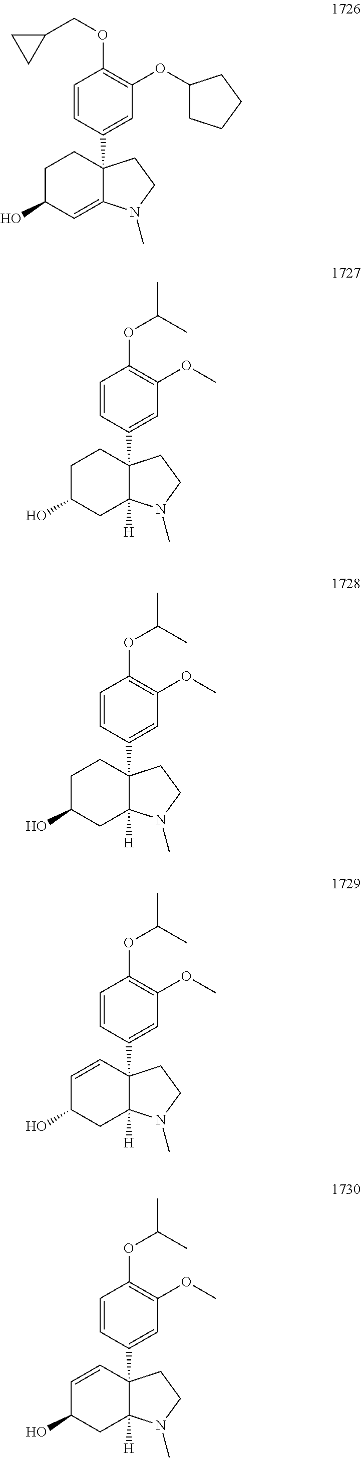

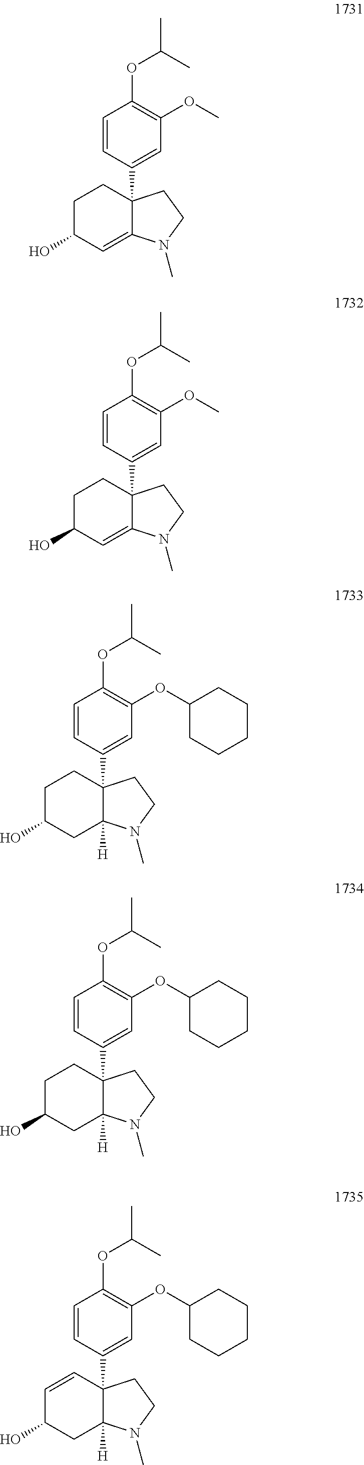

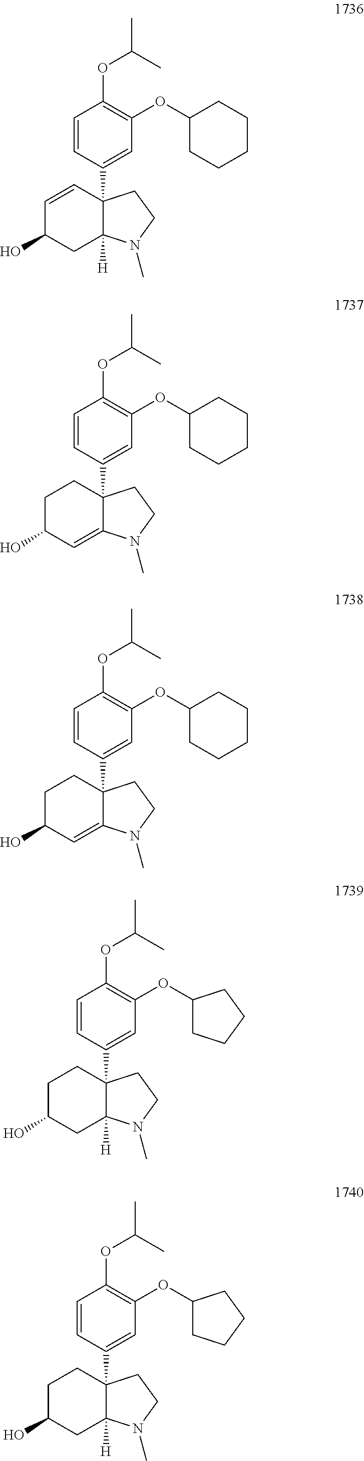

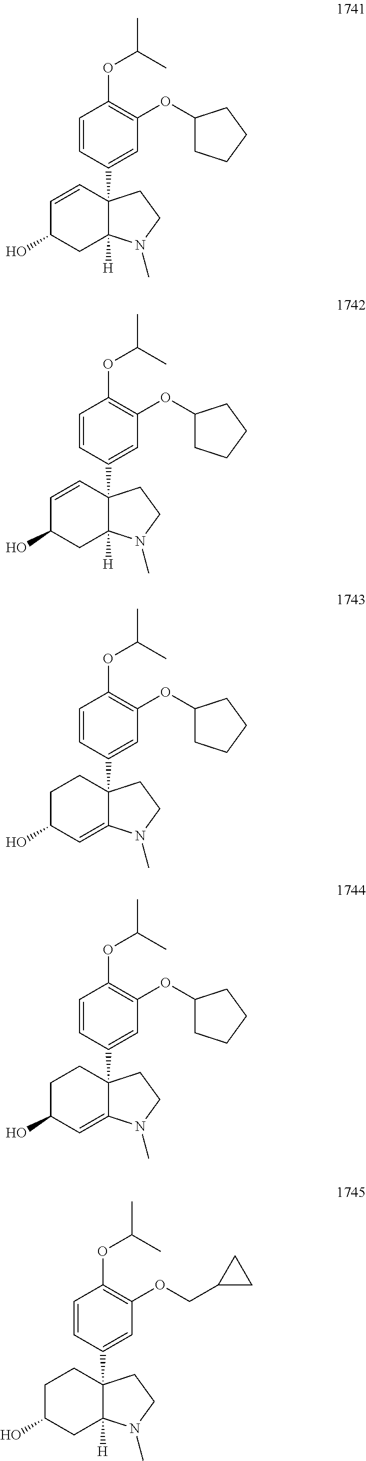

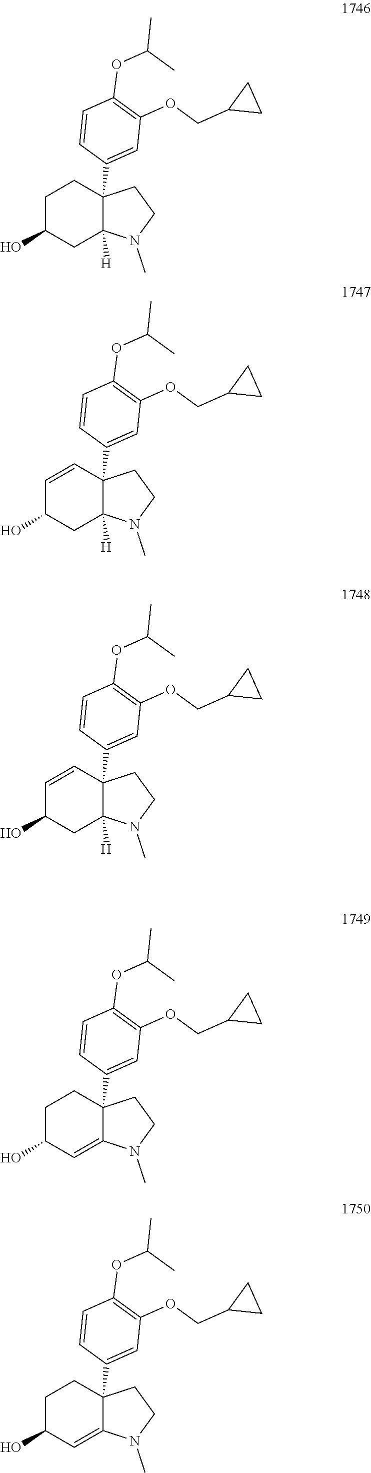

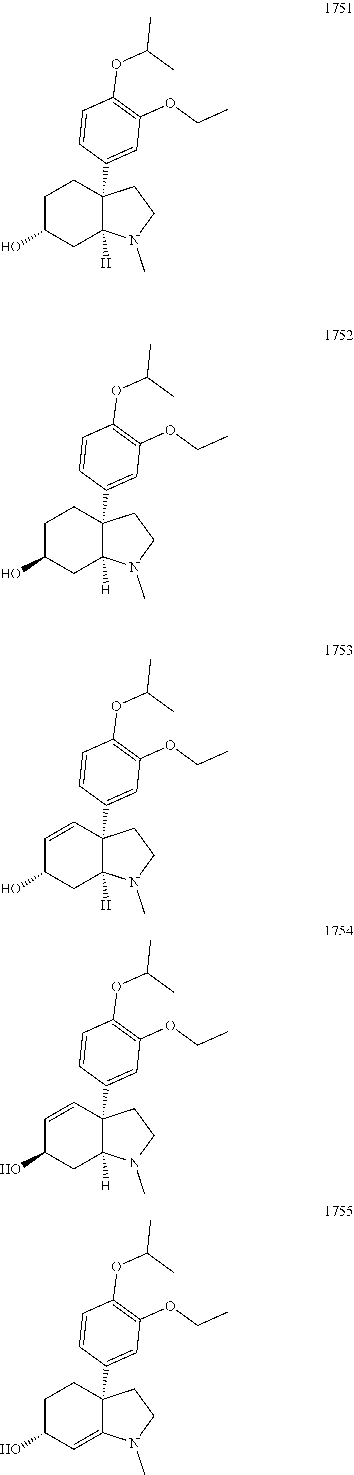

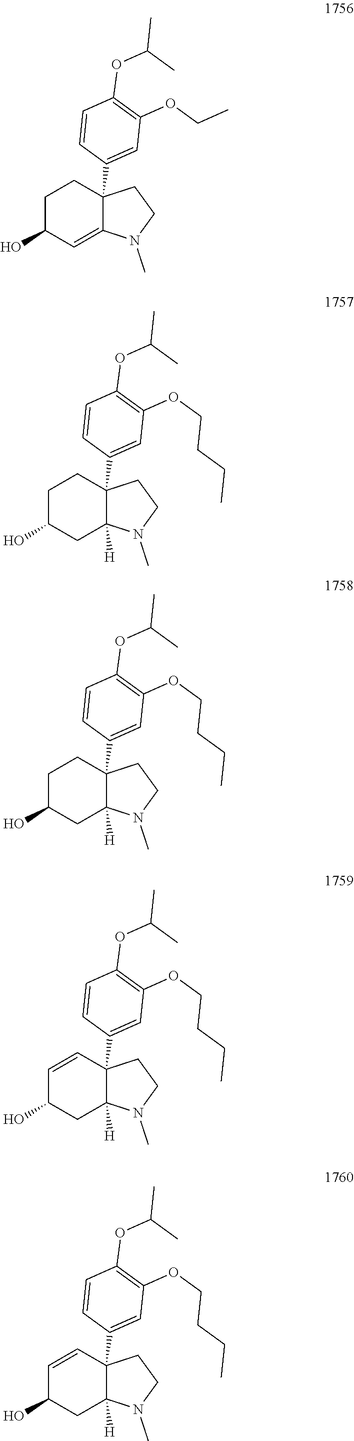

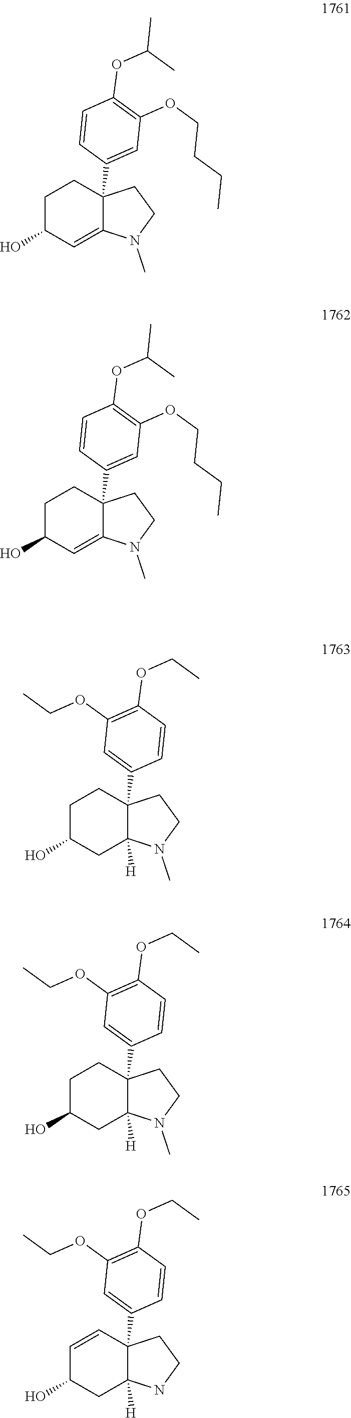

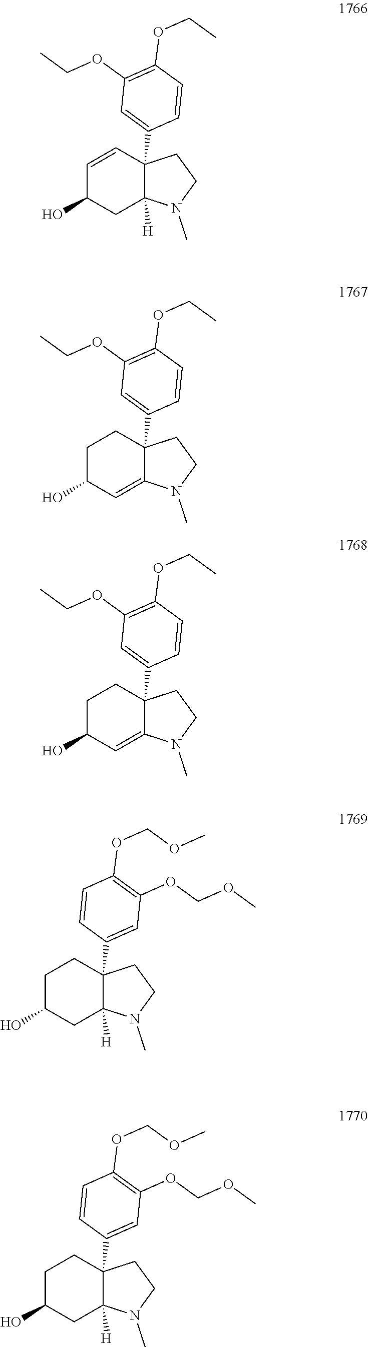

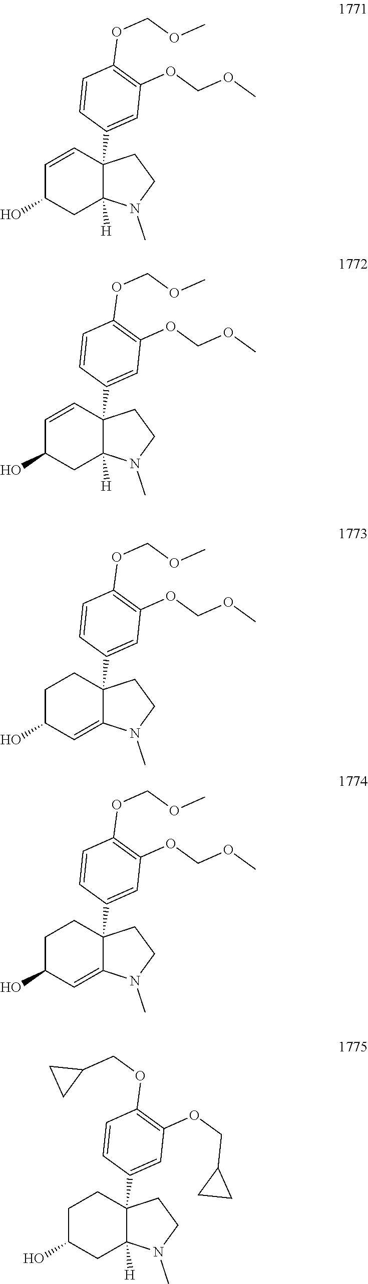

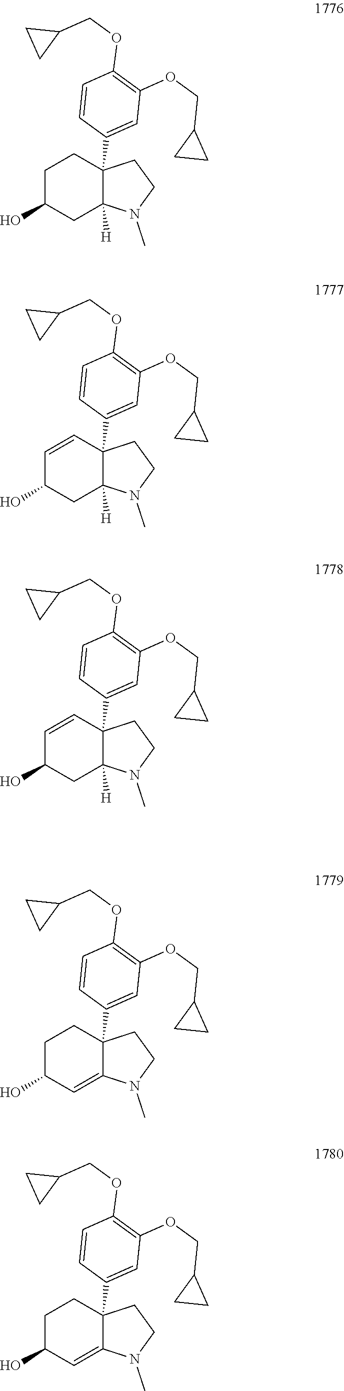 [SMILES: CN1CC[C@]2(c3ccc(OCC4CC4)c(OCC4CC4)c3)CC[C@H](O)C[C@H]12 ; CN1CC[C@]2(c3ccc(OCC4CC4)c(OCC4CC4)c3)C=C[C@@H](O)C[C@H]12 ; CN1CC[C@]2(c3ccc(OCC4CC4)c(OCC4CC4)c3)C=C[C@H](O)C[C@H]12 ; CN1CC[C@]2(c3ccc(OCC4CC4)c(OCC4CC4)c3)CC[C@@H](O)C=C12 ; CN1CC[C@]2(c3ccc(OCC4CC4)c(OCC4CC4)c3)CC[C@H](O)C=C12]

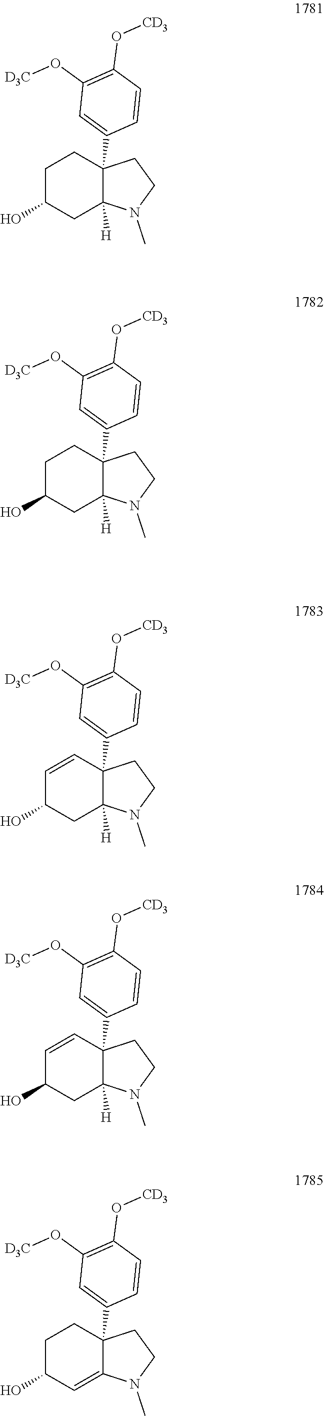 [SMILES: [2H]C([2H])([2H])Oc1ccc([C@@]23CC[C@@H](O)C[C@@H]2N(C)CC3)cc1OC([2H])([2H])[2H] ; [2H]C([2H])([2H])Oc1ccc([C@@]23CC[C@H](O)C[C@@H]2N(C)CC3)cc1OC([2H])([2H])[2H] ; [2H]C([2H])([2H])Oc1ccc([C@@]23C=C[C@@H](O)C[C@@H]2N(C)CC3)cc1OC([2H])([2H])[2H] ; [2H]C([2H])([2H])Oc1ccc([C@@]23C=C[C@H](O)C[C@@H]2N(C)CC3)cc1OC([2H])([2H])[2H] ; [2H]C([2H])([2H])Oc1ccc([C@@]23CC[C@@H](O)C=C2N(C)CC3)cc1OC([2H])([2H])[2H]]

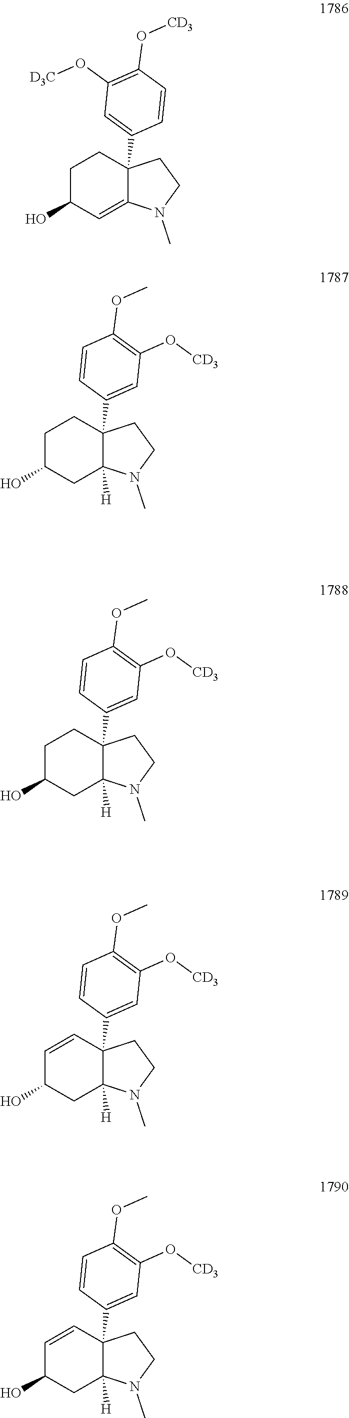 [SMILES: [2H]C([2H])([2H])Oc1ccc([C@@]23CC[C@H](O)C=C2N(C)CC3)cc1OC([2H])([2H])[2H] ; [2H]C([2H])([2H])Oc1cc([C@@]23CC[C@@H](O)C[C@@H]2N(C)CC3)ccc1OC ; [2H]C([2H])([2H])Oc1cc([C@@]23CC[C@H](O)C[C@@H]2N(C)CC3)ccc1OC ; [2H]C([2H])([2H])Oc1cc([C@@]23C=C[C@@H](O)C[C@@H]2N(C)CC3)ccc1OC ; [2H]C([2H])([2H])Oc1cc([C@@]23C=C[C@H](O)C[C@@H]2N(C)CC3)ccc1OC]

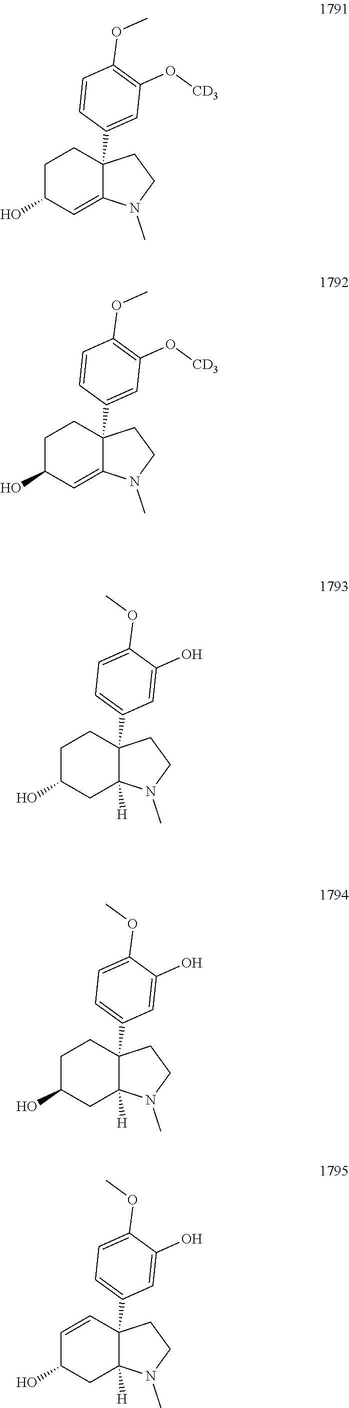 [SMILES: [2H]C([2H])([2H])Oc1cc([C@@]23CC[C@@H](O)C=C2N(C)CC3)ccc1OC ; [2H]C([2H])([2H])Oc1cc([C@@]23CC[C@H](O)C=C2N(C)CC3)ccc1OC ; COc1ccc([C@@]23CC[C@@H](O)C[C@@H]2N(C)CC3)cc1O ; COc1ccc([C@@]23CC[C@H](O)C[C@@H]2N(C)CC3)cc1O ; COc1ccc([C@@]23C=C[C@@H](O)C[C@@H]2N(C)CC3)cc1O]

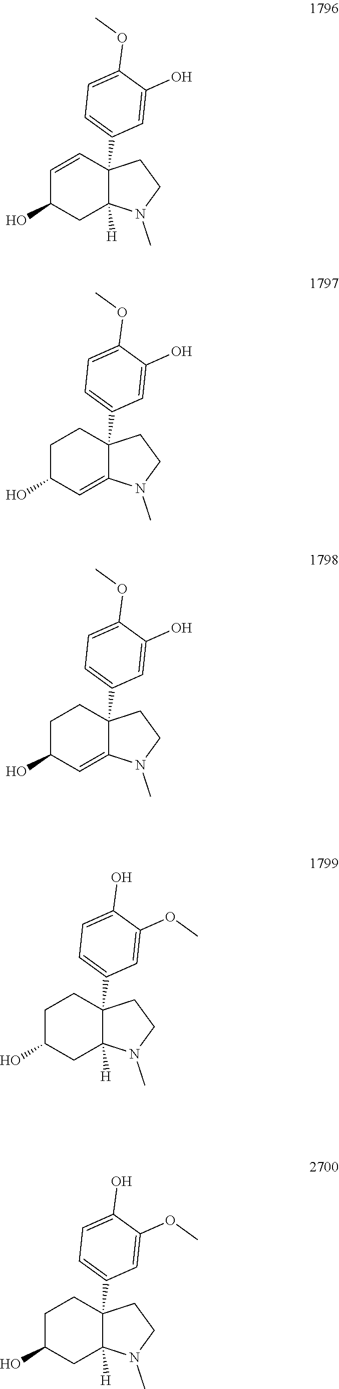 [SMILES: COc1ccc([C@@]23C=C[C@H](O)C[C@@H]2N(C)CC3)cc1O ; COc1ccc([C@@]23CC[C@@H](O)C=C2N(C)CC3)cc1O ; COc1ccc([C@@]23CC[C@H](O)C=C2N(C)CC3)cc1O ; COc1cc([C@@]23CC[C@@H](O)C[C@@H]2N(C)CC3)ccc1O ; COc1cc([C@@]23CC[C@H](O)C[C@@H]2N(C)CC3)ccc1O]

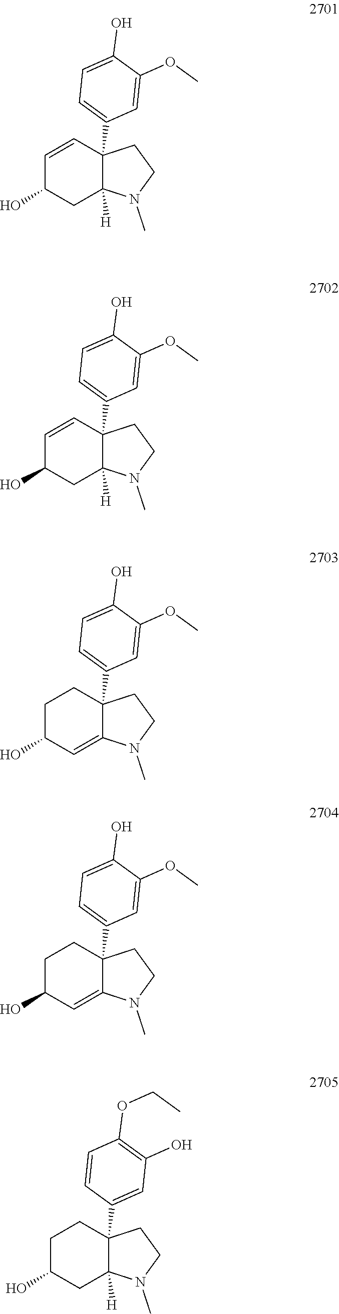 [SMILES: COc1cc([C@@]23C=C[C@@H](O)C[C@@H]2N(C)CC3)ccc1O ; COc1cc([C@@]23C=C[C@H](O)C[C@@H]2N(C)CC3)ccc1O ; COc1cc([C@@]23CC[C@@H](O)C=C2N(C)CC3)ccc1O ; COc1cc([C@@]23CC[C@H](O)C=C2N(C)CC3)ccc1O ; CCOc1ccc([C@@]23CC[C@@H](O)C[C@@H]2N(C)CC3)cc1O]

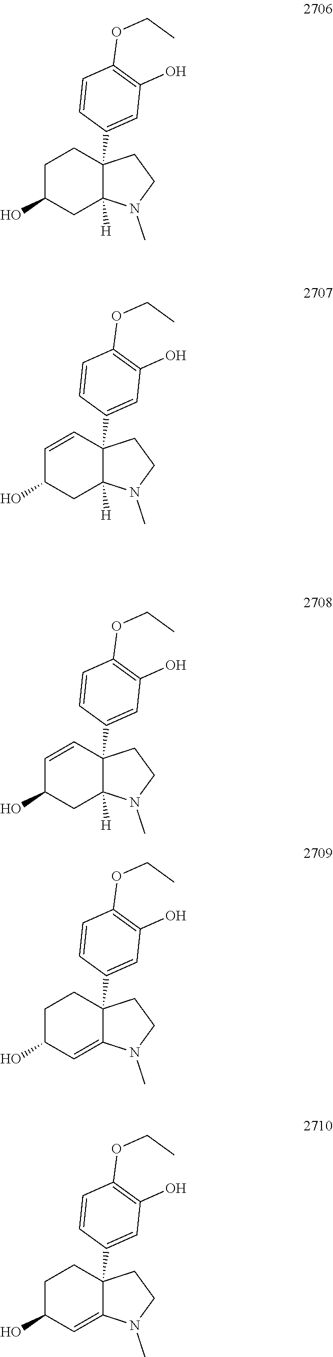

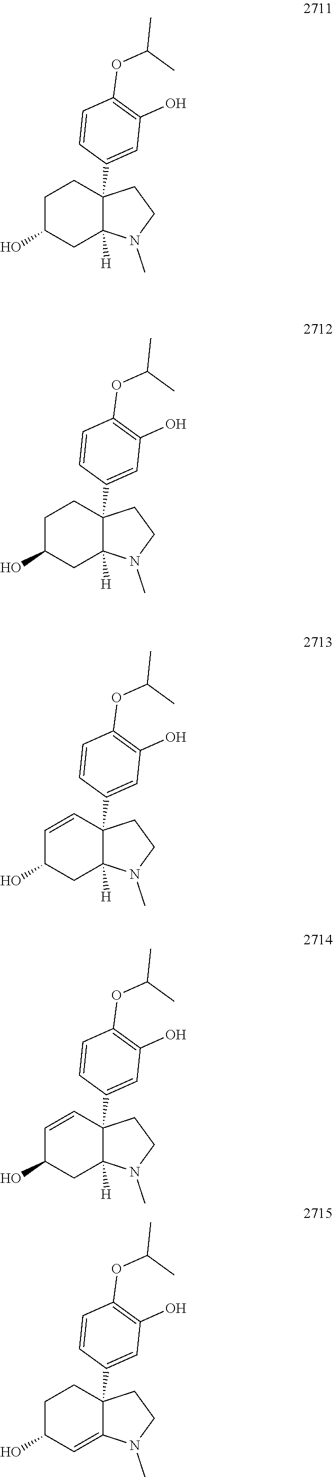

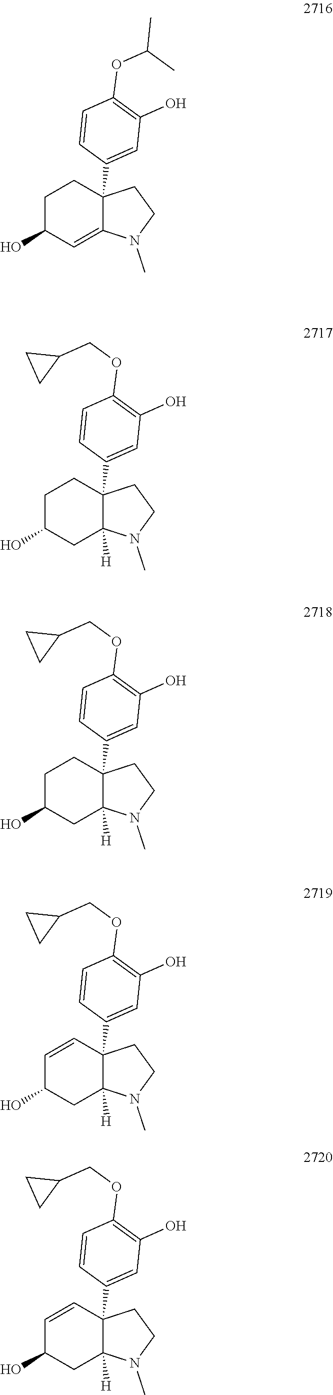 [SMILES: CC(C)Oc1ccc([C@@]23CC[C@H](O)C=C2N(C)CC3)cc1O ; CN1CC[C@]2(c3ccc(OCC4CC4)c(O)c3)CC[C@@H](O)C[C@H]12 ; CN1CC[C@]2(c3ccc(OCC4CC4)c(O)c3)CC[C@H](O)C[C@H]12 ; CN1CC[C@]2(c3ccc(OCC4CC4)c(O)c3)C=C[C@@H](O)C[C@H]12 ; CN1CC[C@]2(c3ccc(OCC4CC4)c(O)c3)C=C[C@H](O)C[C@H]12]

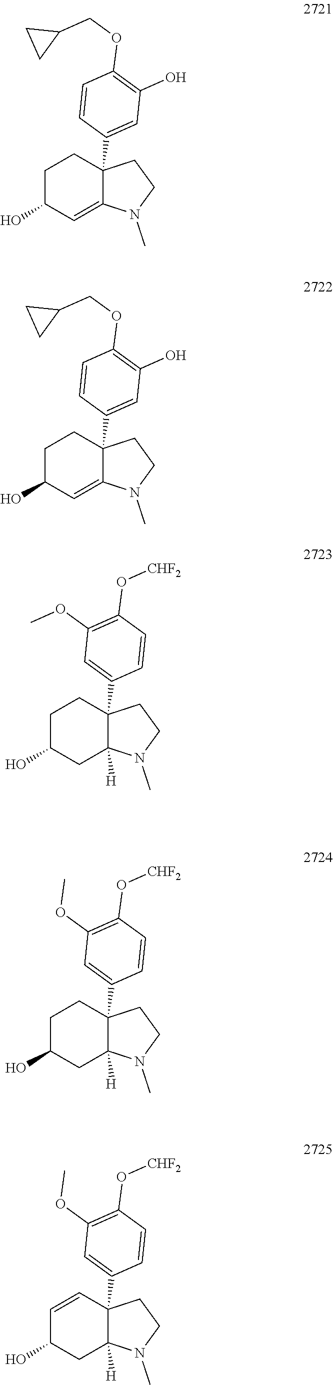

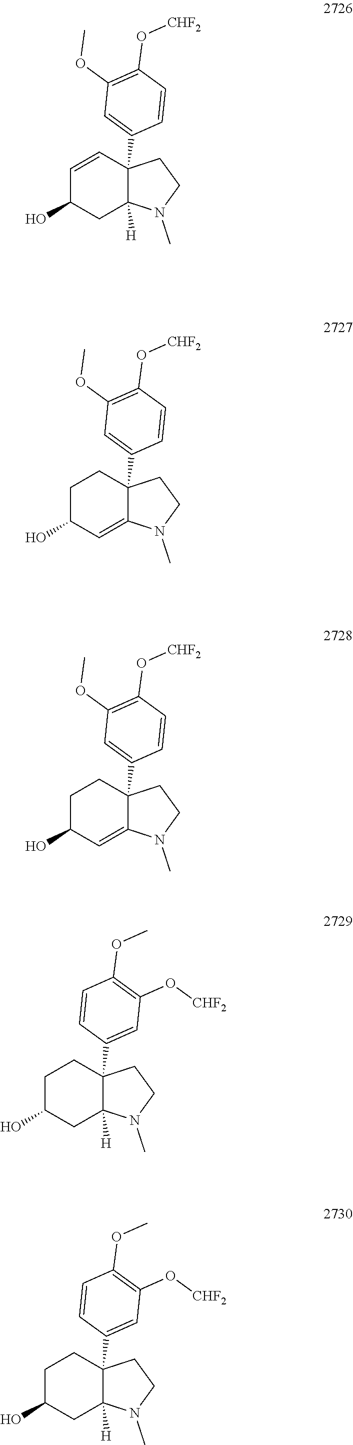 [SMILES: COc1cc([C@@]23C=C[C@H](O)C[C@@H]2N(C)CC3)ccc1OC(F)F ; COc1cc([C@@]23CC[C@@H](O)C=C2N(C)CC3)ccc1OC(F)F ; COc1cc([C@@]23CC[C@H](O)C=C2N(C)CC3)ccc1OC(F)F ; COc1ccc([C@@]23CC[C@@H](O)C[C@@H]2N(C)CC3)cc1OC(F)F ; COc1ccc([C@@]23CC[C@H](O)C[C@@H]2N(C)CC3)cc1OC(F)F]

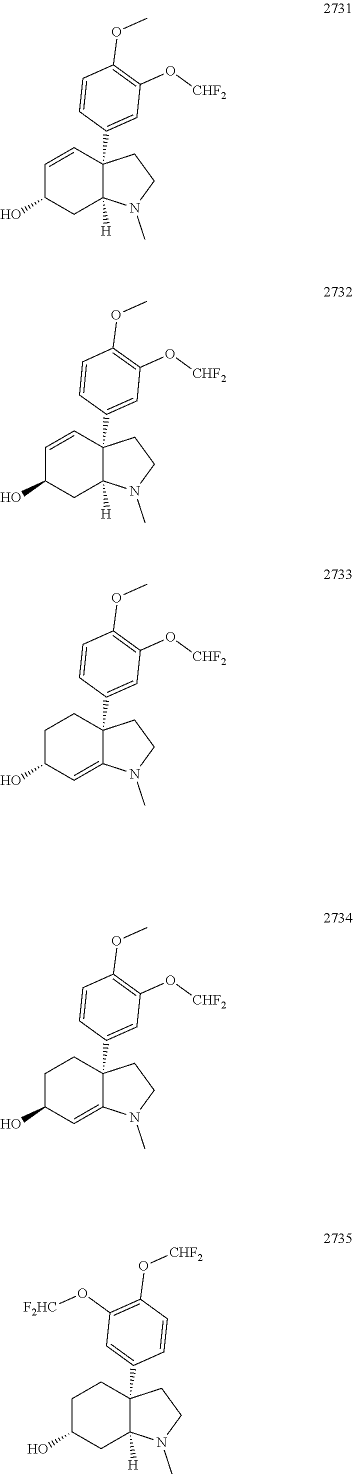 [SMILES: COc1ccc([C@@]23C=C[C@@H](O)C[C@@H]2N(C)CC3)cc1OC(F)F ; COc1ccc([C@@]23C=C[C@H](O)C[C@@H]2N(C)CC3)cc1OC(F)F ; COc1ccc([C@@]23CC[C@@H](O)C=C2N(C)CC3)cc1OC(F)F ; COc1ccc([C@@]23CC[C@H](O)C=C2N(C)CC3)cc1OC(F)F ; CN1CC[C@]2(c3ccc(OC(F)F)c(OC(F)F)c3)CC[C@@H](O)C[C@H]12]

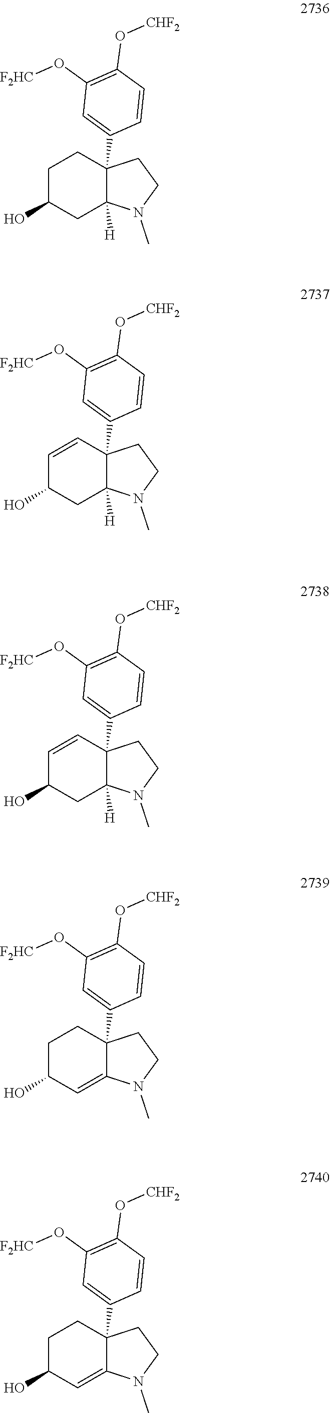 [SMILES: CN1CC[C@]2(c3ccc(OC(F)F)c(OC(F)F)c3)CC[C@H](O)C[C@H]12 ; CN1CC[C@]2(c3ccc(OC(F)F)c(OC(F)F)c3)C=C[C@@H](O)C[C@H]12 ; CN1CC[C@]2(c3ccc(OC(F)F)c(OC(F)F)c3)C=C[C@H](O)C[C@H]12 ; CN1CC[C@]2(c3ccc(OC(F)F)c(OC(F)F)c3)CC[C@@H](O)C=C12 ; CN1CC[C@]2(c3ccc(OC(F)F)c(OC(F)F)c3)CC[C@H](O)C=C12]

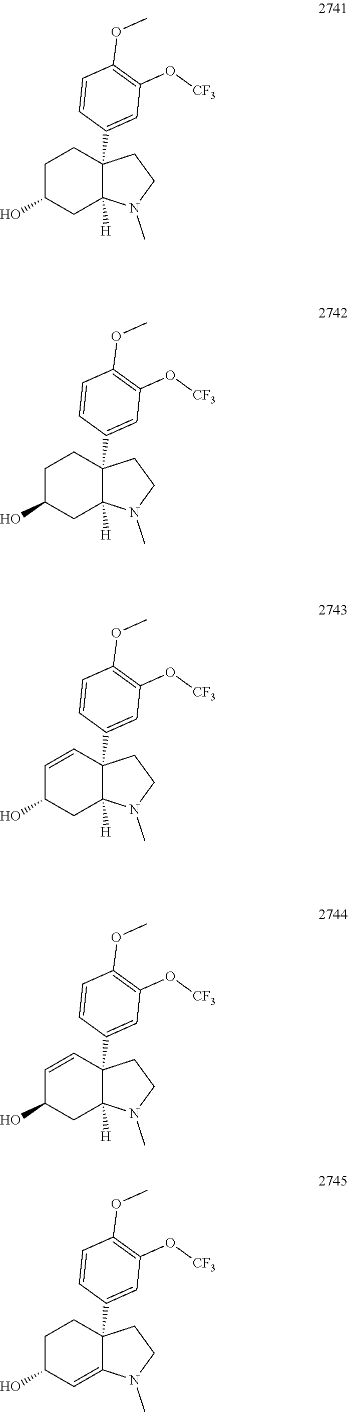

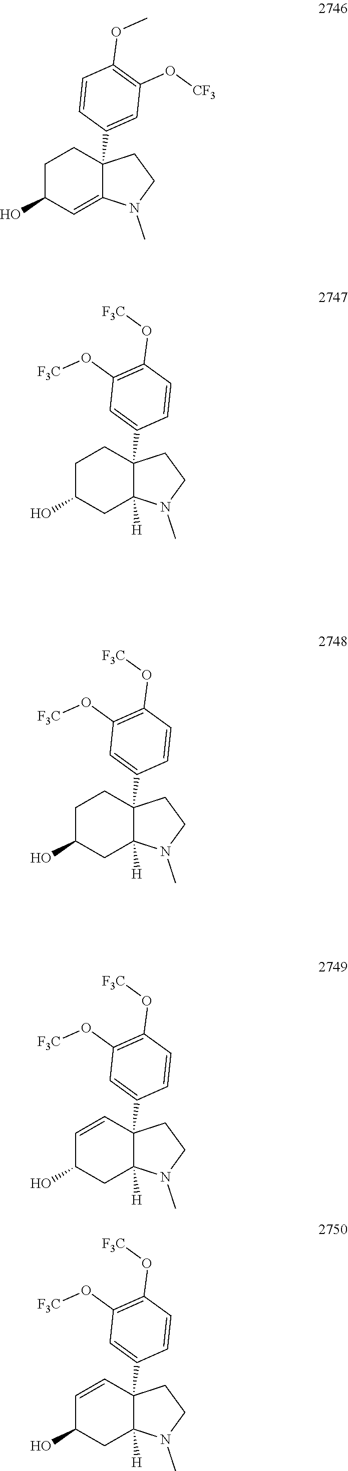 [SMILES: COc1ccc([C@@]23CC[C@H](O)C=C2N(C)CC3)cc1OC(F)(F)F ; CN1CC[C@]2(c3ccc(OC(F)(F)F)c(OC(F)(F)F)c3)CC[C@@H](O)C[C@H]12 ; CN1CC[C@]2(c3ccc(OC(F)(F)F)c(OC(F)(F)F)c3)CC[C@H](O)C[C@H]12 ; CN1CC[C@]2(c3ccc(OC(F)(F)F)c(OC(F)(F)F)c3)C=C[C@@H](O)C[C@H]12 ; CN1CC[C@]2(c3ccc(OC(F)(F)F)c(OC(F)(F)F)c3)C=C[C@H](O)C[C@H]12]

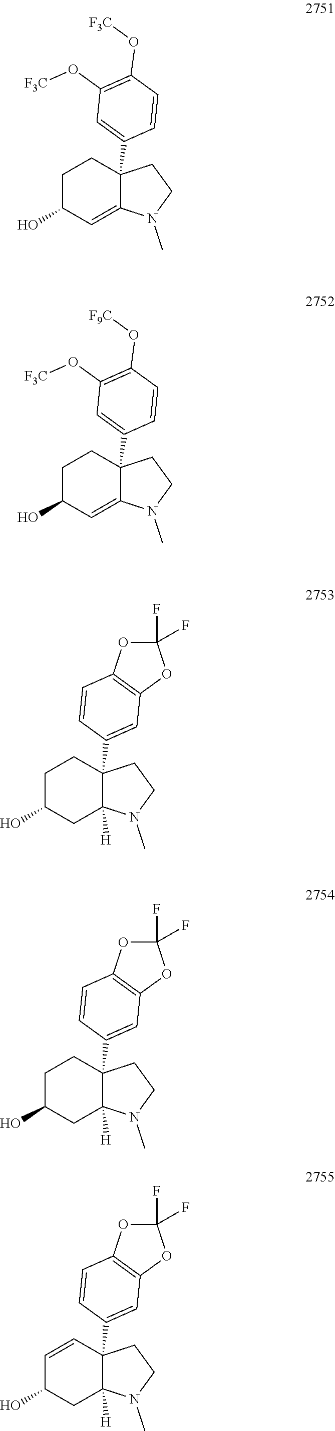

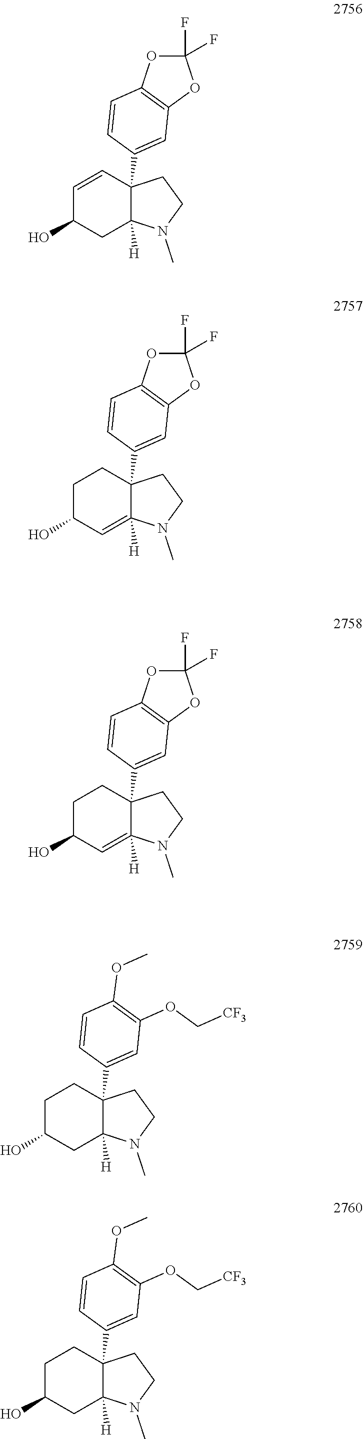

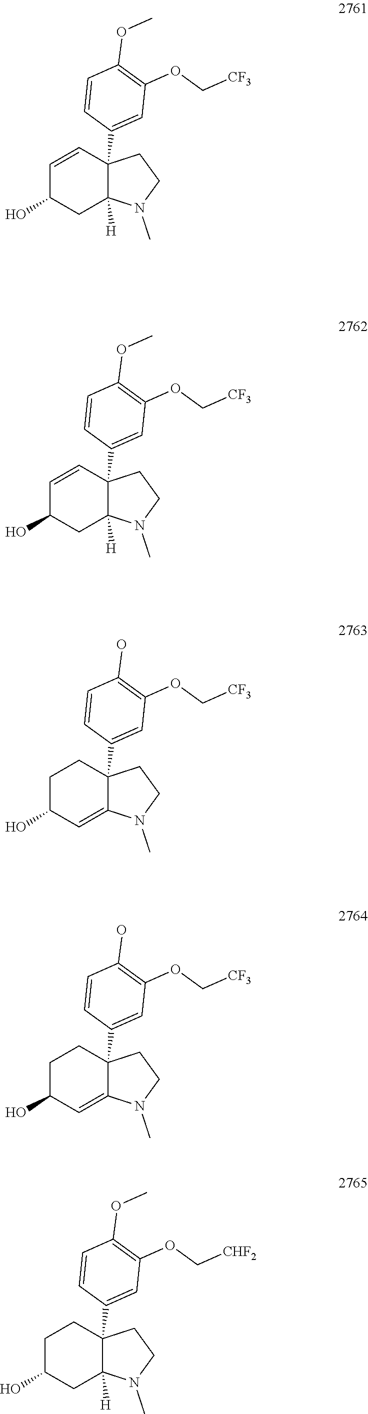

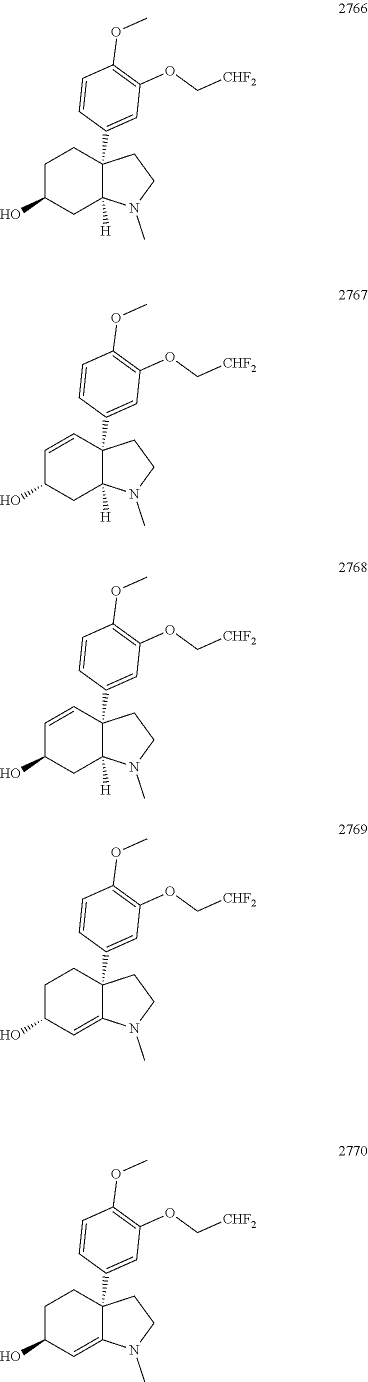

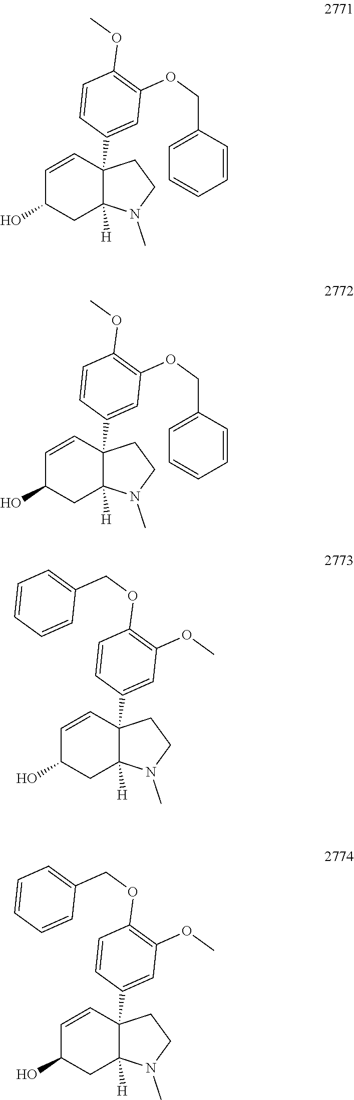 [SMILES: COc1ccc([C@@]23C=C[C@@H](O)C[C@@H]2N(C)CC3)cc1OCc1ccccc1 ; COc1ccc([C@@]23C=C[C@H](O)C[C@@H]2N(C)CC3)cc1OCc1ccccc1 ; COc1cc([C@@]23C=C[C@@H](O)C[C@@H]2N(C)CC3)ccc1OCc1ccccc1 ; COc1cc([C@@]23C=C[C@H](O)C[C@@H]2N(C)CC3)ccc1OCc1ccccc1]

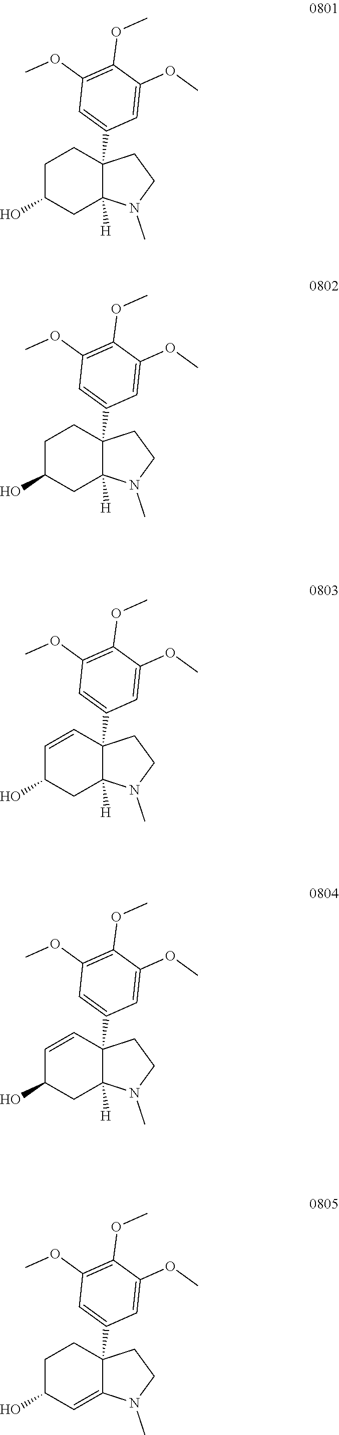 [SMILES: COc1cc([C@@]23CC[C@@H](O)C[C@@H]2N(C)CC3)cc(OC)c1OC ; COc1cc([C@@]23CC[C@H](O)C[C@@H]2N(C)CC3)cc(OC)c1OC ; COc1cc([C@@]23C=C[C@@H](O)C[C@@H]2N(C)CC3)cc(OC)c1OC ; COc1cc([C@@]23C=C[C@H](O)C[C@@H]2N(C)CC3)cc(OC)c1OC ; COc1cc([C@@]23CC[C@@H](O)C=C2N(C)CC3)cc(OC)c1OC]

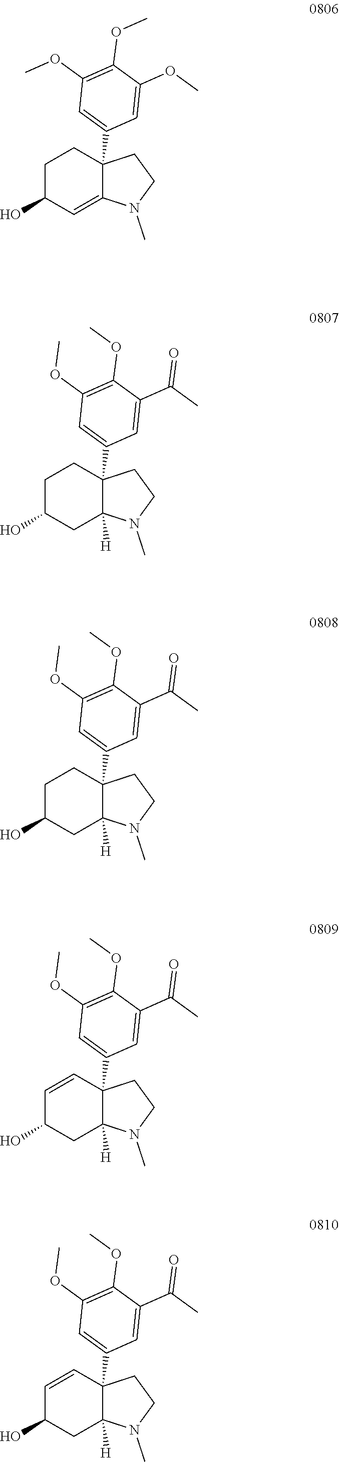 [SMILES: COc1cc([C@@]23CC[C@H](O)C=C2N(C)CC3)cc(OC)c1OC ; COc1cc([C@@]23CC[C@@H](O)C[C@@H]2N(C)CC3)cc(C(C)=O)c1OC ; COc1cc([C@@]23CC[C@H](O)C[C@@H]2N(C)CC3)cc(C(C)=O)c1OC ; COc1cc([C@@]23C=C[C@@H](O)C[C@@H]2N(C)CC3)cc(C(C)=O)c1OC ; COc1cc([C@@]23C=C[C@H](O)C[C@@H]2N(C)CC3)cc(C(C)=O)c1OC]

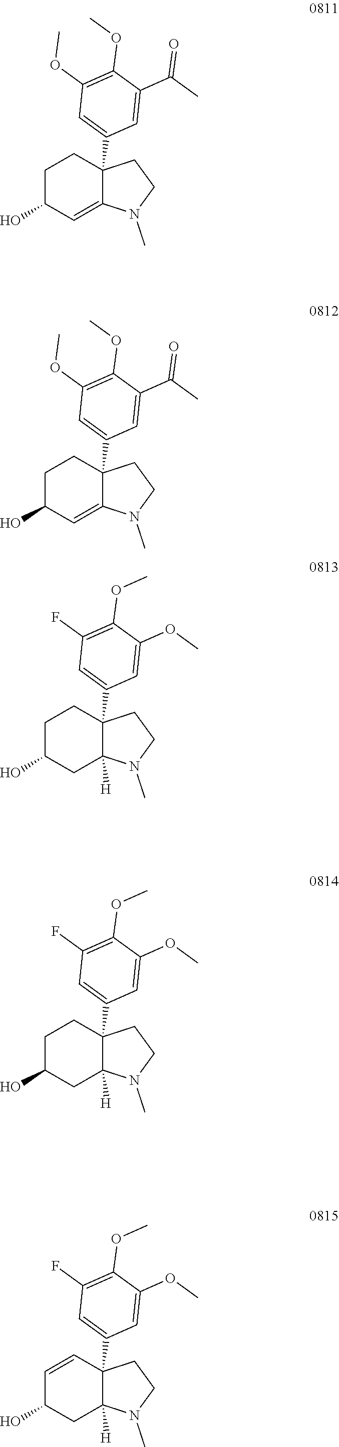

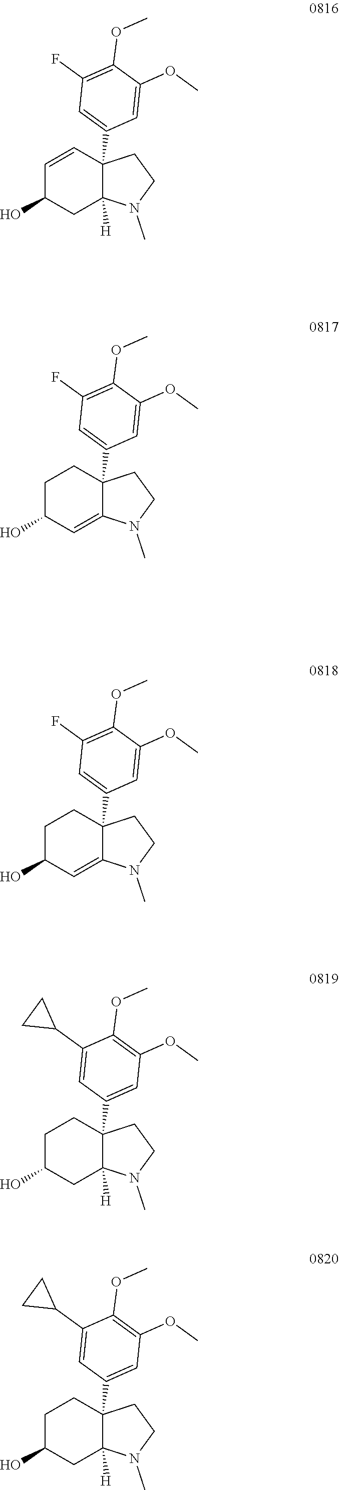 [SMILES: COc1cc([C@@]23C=C[C@H](O)C[C@@H]2N(C)CC3)cc(F)c1OC ; COc1cc([C@@]23CC[C@@H](O)C=C2N(C)CC3)cc(F)c1OC ; COc1cc([C@@]23CC[C@H](O)C=C2N(C)CC3)cc(F)c1OC ; COc1cc([C@@]23CC[C@@H](O)C[C@@H]2N(C)CC3)cc(C2CC2)c1OC ; COc1cc([C@@]23CC[C@H](O)C[C@@H]2N(C)CC3)cc(C2CC2)c1OC]

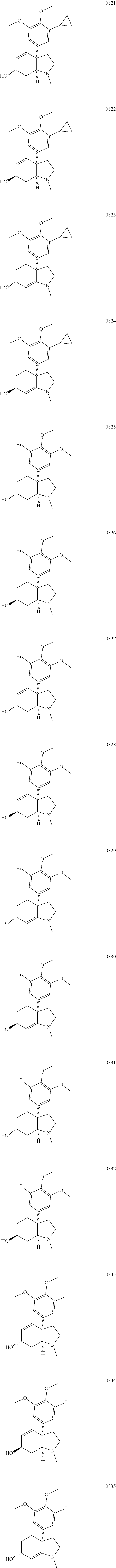

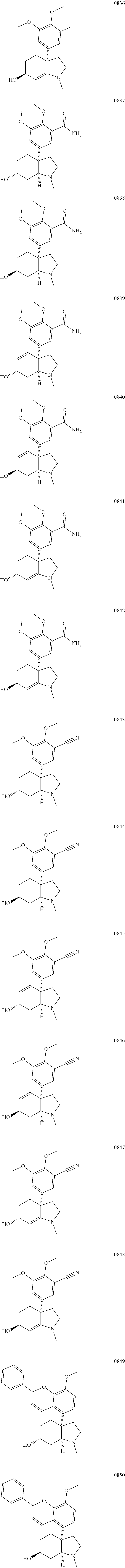

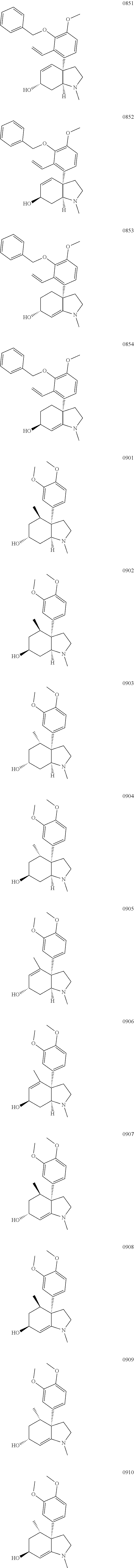

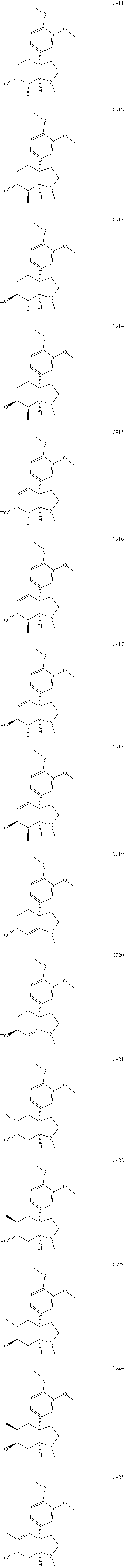

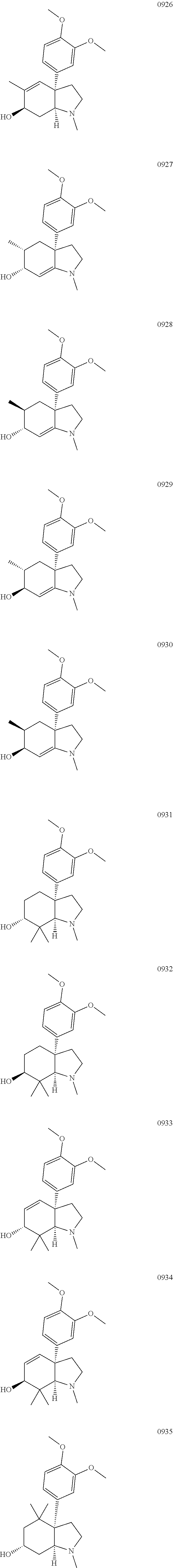

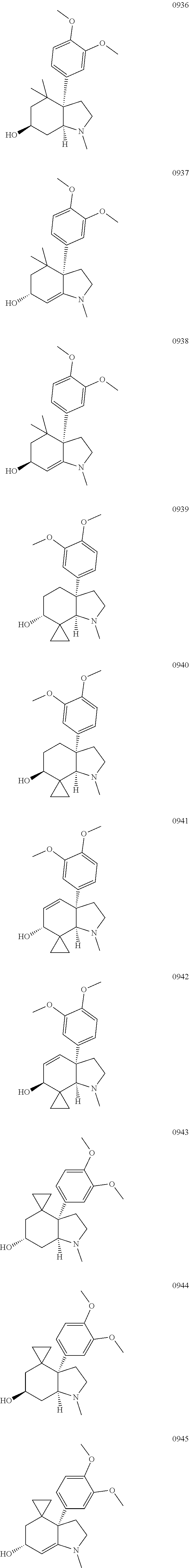

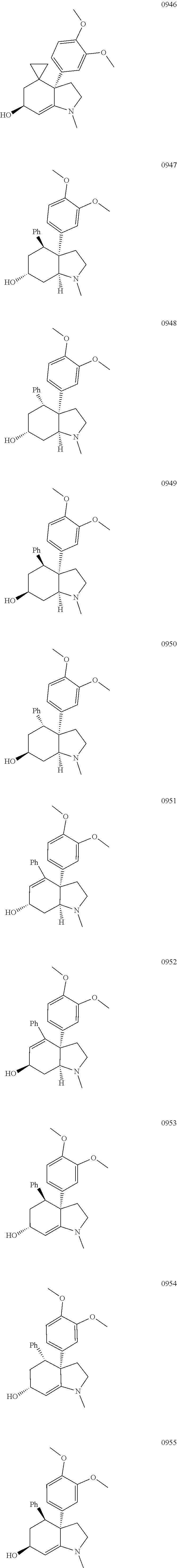

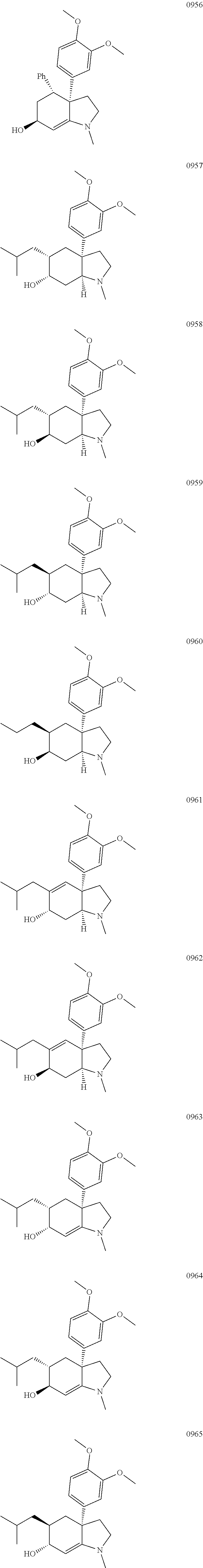

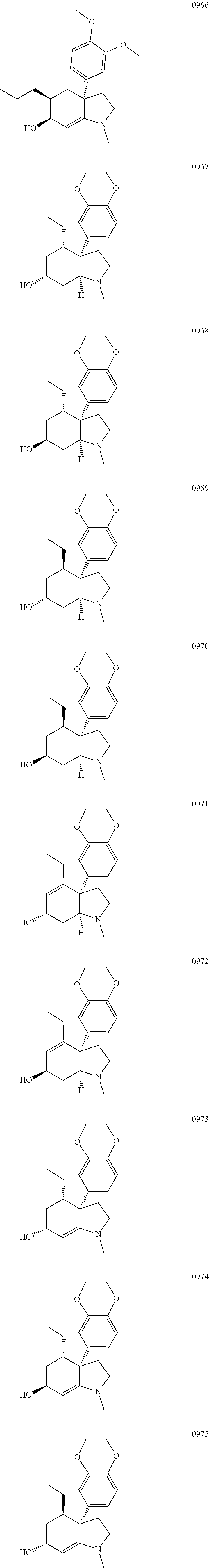

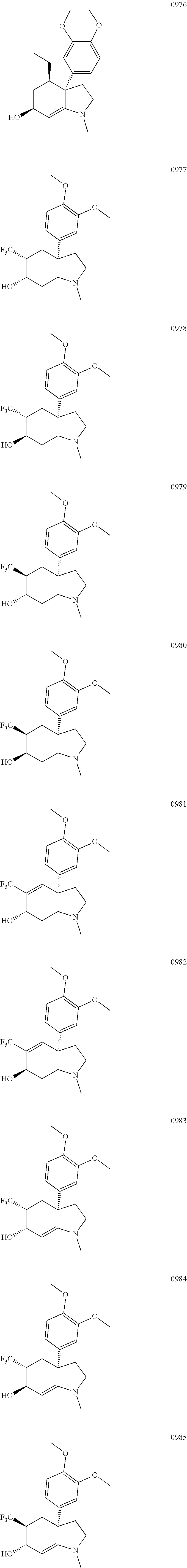

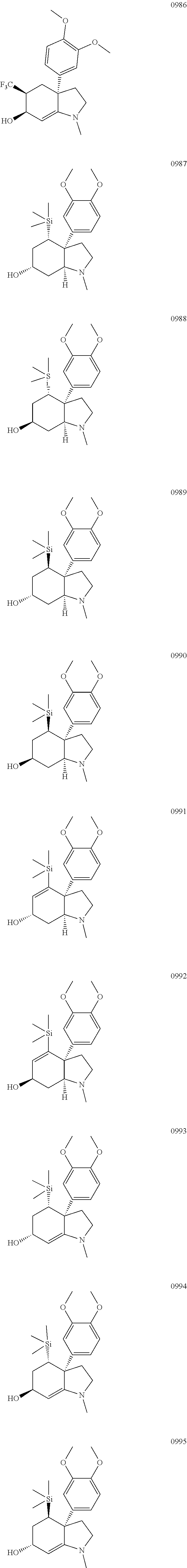

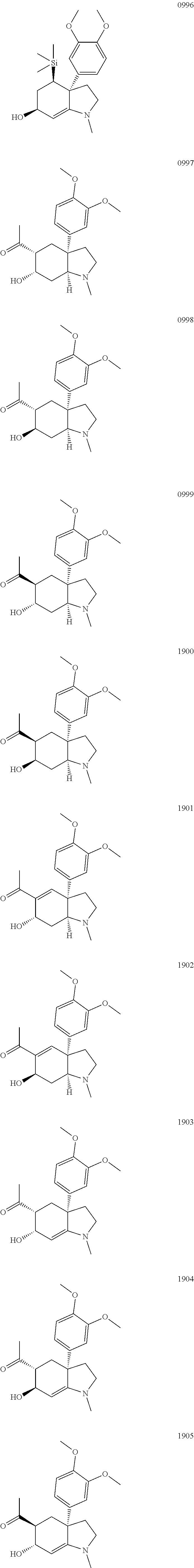

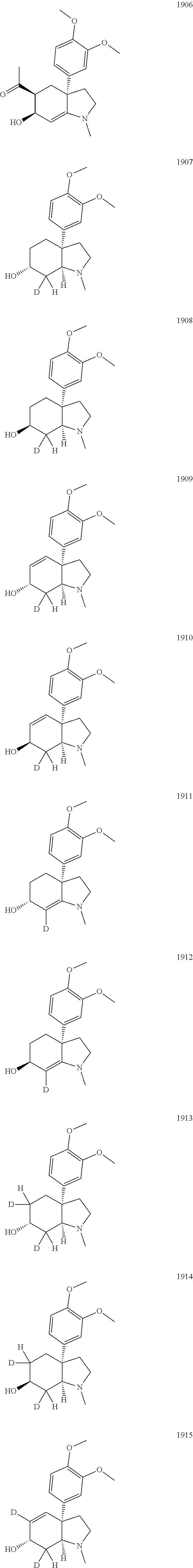

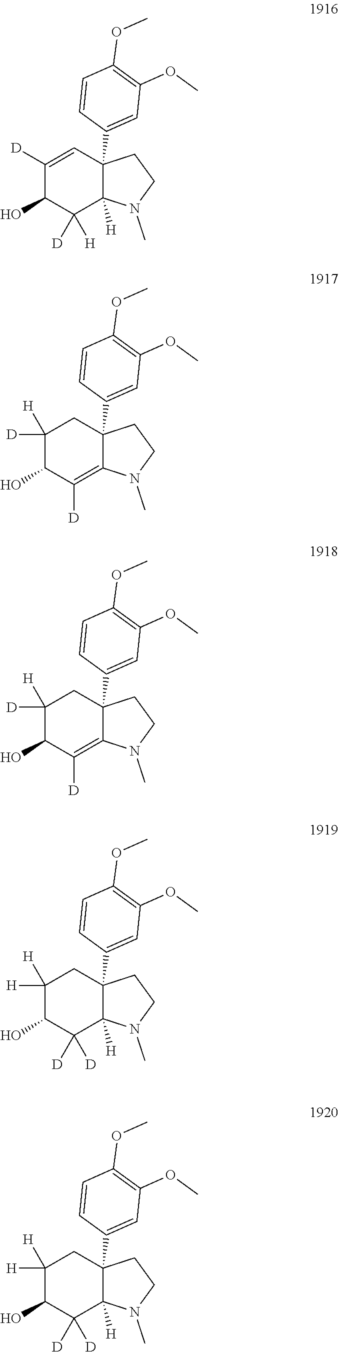 [SMILES: [2H]C1=C[C@@]2(c3ccc(OC)c(OC)c3)CCN(C)[C@H]2C([2H])[C@H]1O ; [2H]C1=C2N(C)CC[C@]2(c2ccc(OC)c(OC)c2)CC([2H])[C@H]1O ; [2H]C1=C2N(C)CC[C@]2(c2ccc(OC)c(OC)c2)CC([2H])[C@@H]1O ; [2H]C1([2H])[C@H](O)CC[C@@]2(c3ccc(OC)c(OC)c3)CCN(C)[C@@H]12 ; [2H]C1([2H])[C@@H](O)CC[C@@]2(c3ccc(OC)c(OC)c3)CCN(C)[C@@H]12]

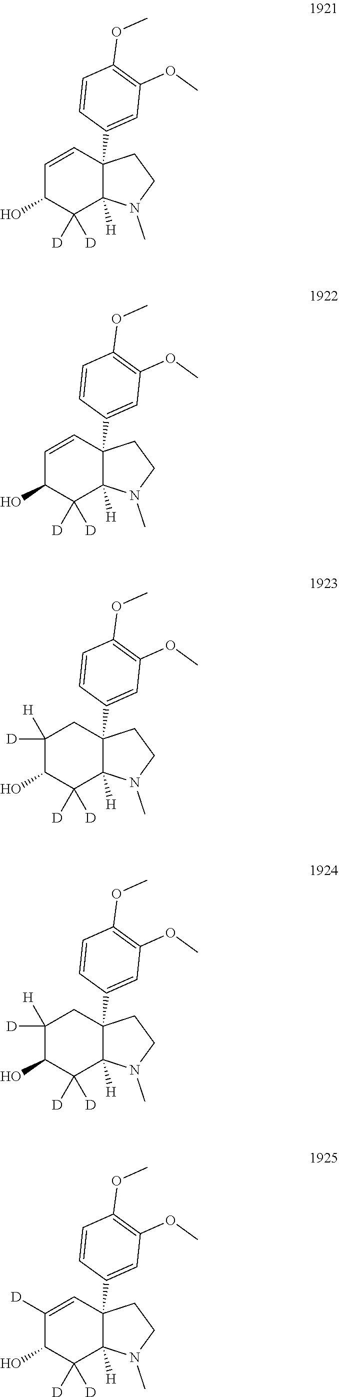 [SMILES: [2H]C1([2H])[C@H](O)C=C[C@@]2(c3ccc(OC)c(OC)c3)CCN(C)[C@@H]12 ; [2H]C1([2H])[C@@H](O)C=C[C@@]2(c3ccc(OC)c(OC)c3)CCN(C)[C@@H]12 ; [2H]C1C[C@@]2(c3ccc(OC)c(OC)c3)CCN(C)[C@H]2C([2H])([2H])[C@@H]1O ; [2H]C1C[C@@]2(c3ccc(OC)c(OC)c3)CCN(C)[C@H]2C([2H])([2H])[C@H]1O ; [2H]C1=C[C@@]2(c3ccc(OC)c(OC)c3)CCN(C)[C@H]2C([2H])([2H])[C@@H]1O]

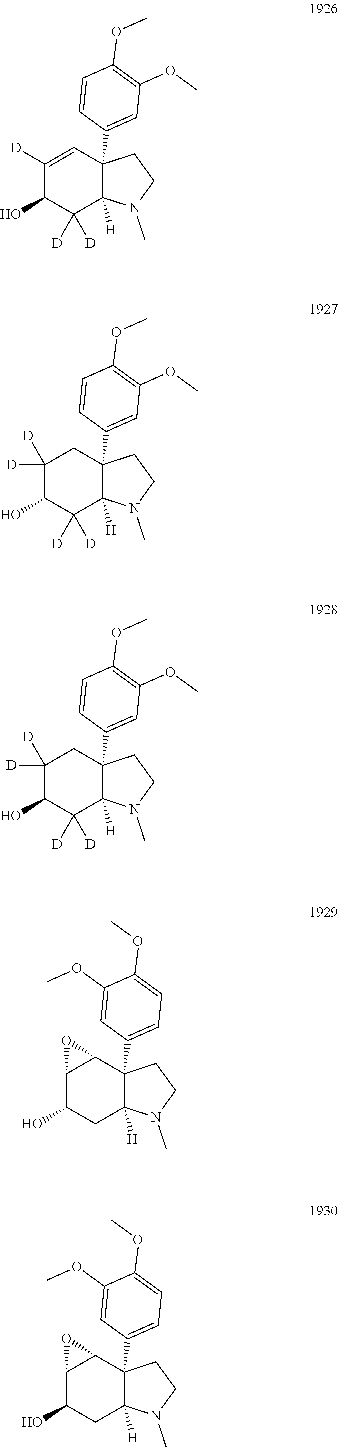 [SMILES: [2H]C1=C[C@@]2(c3ccc(OC)c(OC)c3)CCN(C)[C@H]2C([2H])([2H])[C@H]1O ; [2H]C1([2H])C[C@@]2(c3ccc(OC)c(OC)c3)CCN(C)[C@H]2C([2H])([2H])[C@@H]1O ; [2H]C1([2H])C[C@@]2(c3ccc(OC)c(OC)c3)CCN(C)[C@H]2C([2H])([2H])[C@H]1O ; COc1ccc([C@]23CCN(C)[C@H]2C[C@H](O)[C@@H]2O[C@@H]23)cc1OC ; COc1ccc([C@]23CCN(C)[C@H]2C[C@@H](O)[C@@H]2O[C@@H]23)cc1OC]

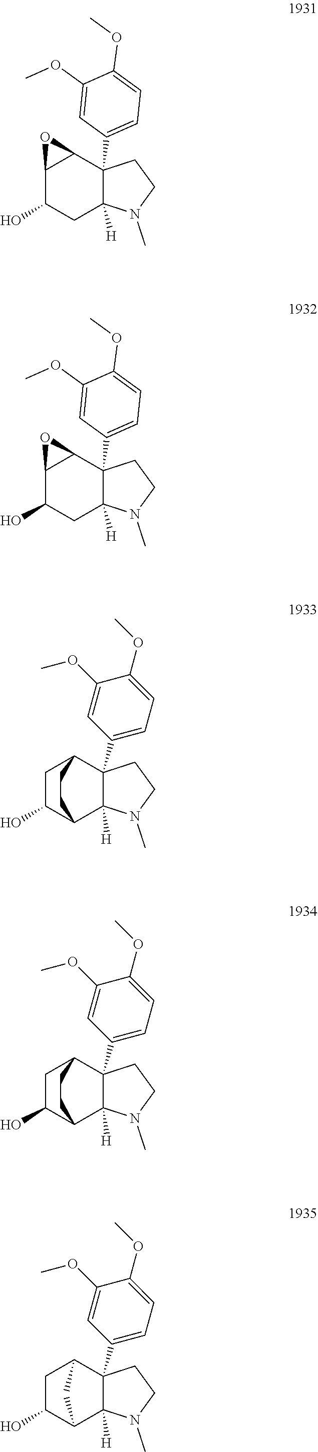 [SMILES: COc1ccc([C@]23CCN(C)[C@H]2C[C@H](O)[C@H]2O[C@H]23)cc1OC ; COc1ccc([C@]23CCN(C)[C@H]2C[C@@H](O)[C@H]2O[C@H]23)cc1OC ; COc1ccc([C@]23CCN(C)[C@H]2[C@H]2CC[C@@H]3C[C@H]2O)cc1OC ; COc1ccc([C@]23CCN(C)[C@H]2[C@H]2CC[C@@H]3C[C@@H]2O)cc1OC ; COc1ccc([C@]23CCN(C)[C@H]2[C@@H]2C[C@H]3C[C@H]2O)cc1OC]

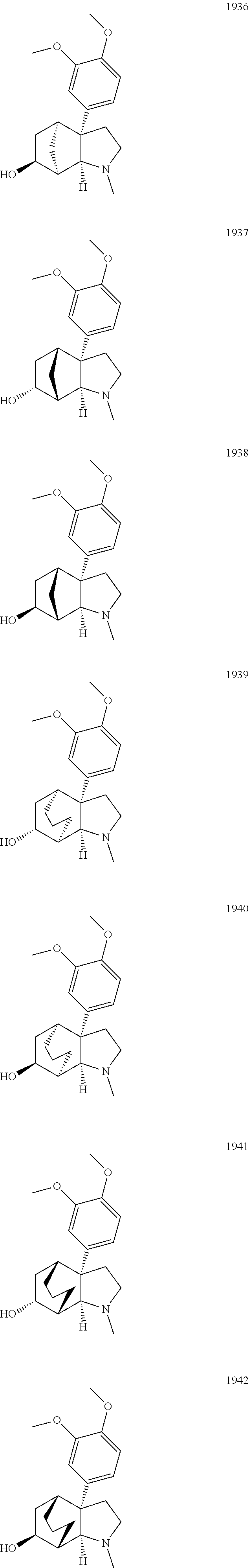

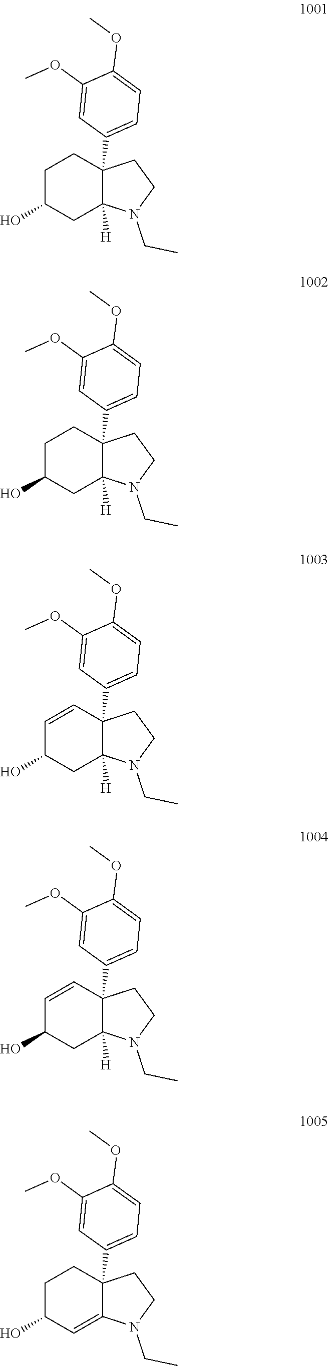 [SMILES: CCN1CC[C@]2(c3ccc(OC)c(OC)c3)CC[C@@H](O)C[C@H]12 ; CCN1CC[C@]2(c3ccc(OC)c(OC)c3)CC[C@H](O)C[C@H]12 ; CCN1CC[C@]2(c3ccc(OC)c(OC)c3)C=C[C@@H](O)C[C@H]12 ; CCN1CC[C@]2(c3ccc(OC)c(OC)c3)C=C[C@H](O)C[C@H]12 ; CCN1CC[C@]2(c3ccc(OC)c(OC)c3)CC[C@@H](O)C=C12]

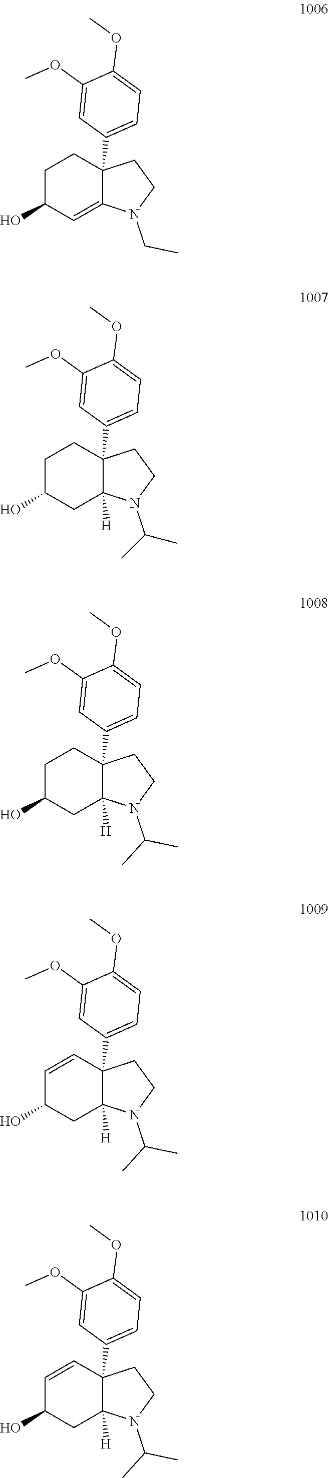 [SMILES: CCN1CC[C@]2(c3ccc(OC)c(OC)c3)CC[C@H](O)C=C12 ; COc1ccc([C@@]23CC[C@@H](O)C[C@@H]2N(C(C)C)CC3)cc1OC ; COc1ccc([C@@]23CC[C@H](O)C[C@@H]2N(C(C)C)CC3)cc1OC ; COc1ccc([C@@]23C=C[C@@H](O)C[C@@H]2N(C(C)C)CC3)cc1OC ; COc1ccc([C@@]23C=C[C@H](O)C[C@@H]2N(C(C)C)CC3)cc1OC]

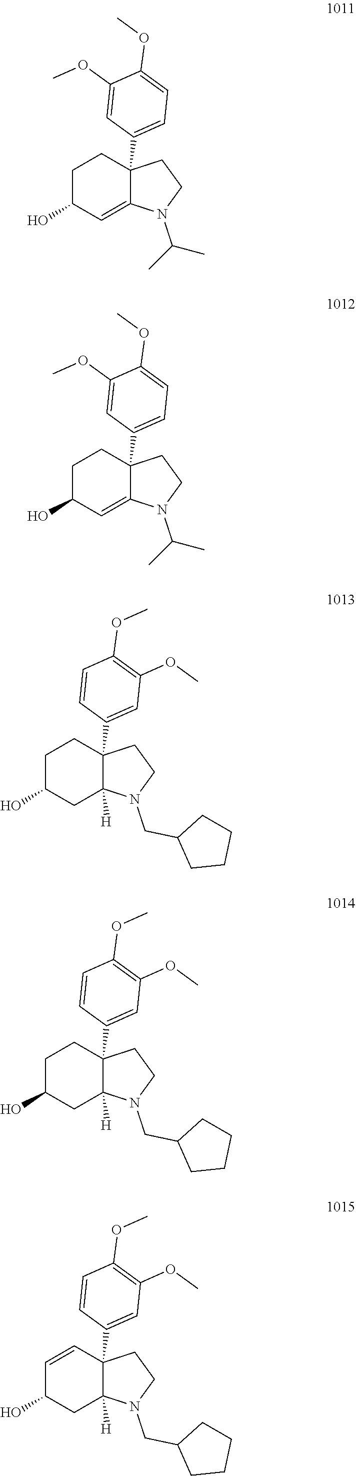

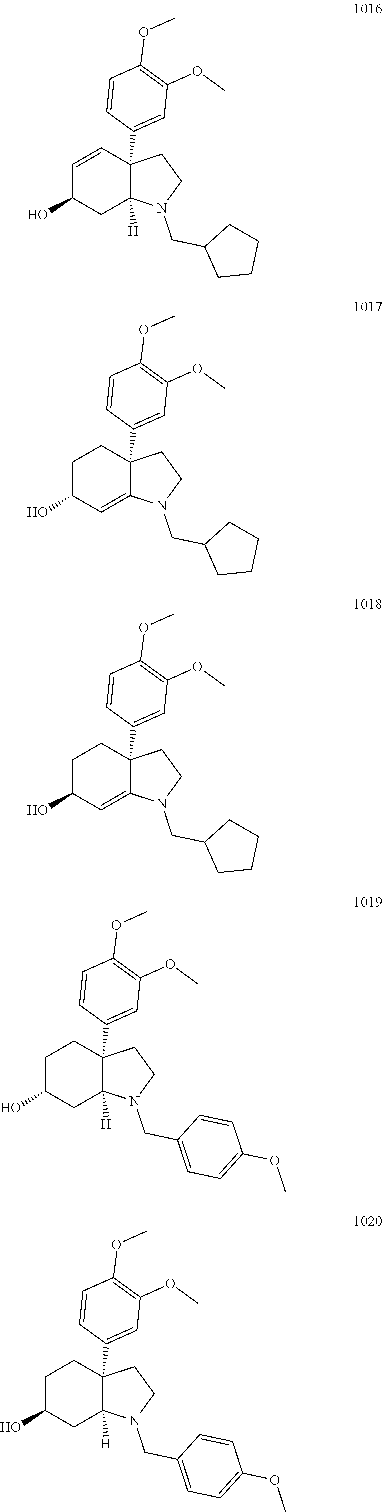 [SMILES: COc1ccc([C@@]23C=C[C@H](O)C[C@@H]2N(CC2CCCC2)CC3)cc1OC ; COc1ccc([C@@]23CC[C@@H](O)C=C2N(CC2CCCC2)CC3)cc1OC ; COc1ccc([C@@]23CC[C@H](O)C=C2N(CC2CCCC2)CC3)cc1OC ; COc1ccc(CN2CC[C@]3(c4ccc(OC)c(OC)c4)CC[C@@H](O)C[C@H]23)cc1 ; COc1ccc(CN2CC[C@]3(c4ccc(OC)c(OC)c4)CC[C@H](O)C[C@H]23)cc1]

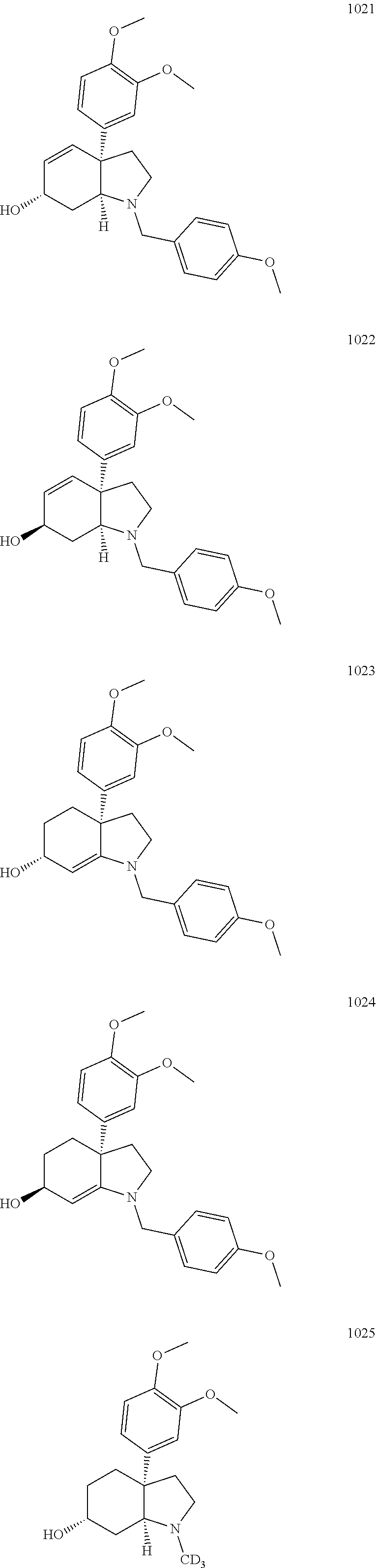 [SMILES: COc1ccc(CN2CC[C@]3(c4ccc(OC)c(OC)c4)C=C[C@@H](O)C[C@H]23)cc1 ; COc1ccc(CN2CC[C@]3(c4ccc(OC)c(OC)c4)C=C[C@H](O)C[C@H]23)cc1 ; COc1ccc(CN2CC[C@]3(c4ccc(OC)c(OC)c4)CC[C@@H](O)C=C23)cc1 ; COc1ccc(CN2CC[C@]3(c4ccc(OC)c(OC)c4)CC[C@H](O)C=C23)cc1 ; [2H]C([2H])([2H])N1CC[C@]2(c3ccc(OC)c(OC)c3)CC[C@@H](O)C[C@H]12]

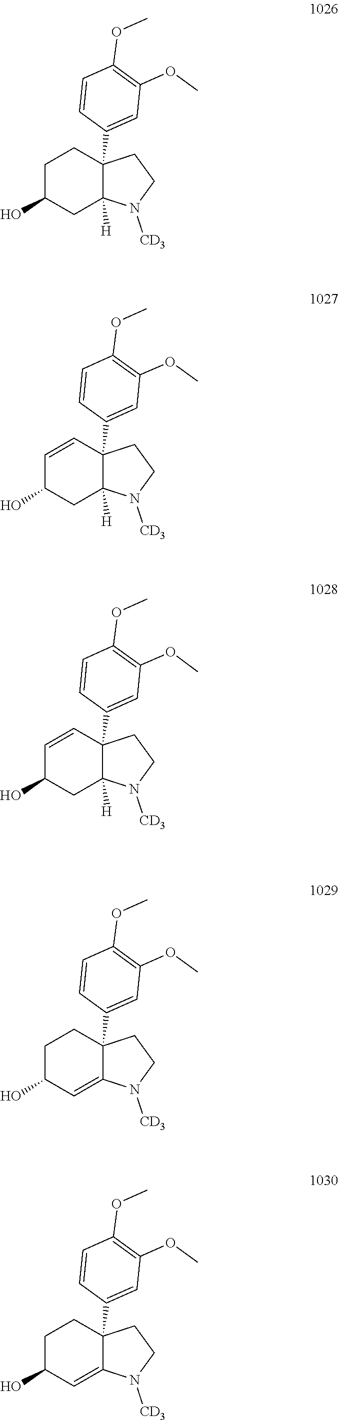 [SMILES: [2H]C([2H])([2H])N1CC[C@]2(c3ccc(OC)c(OC)c3)CC[C@H](O)C[C@H]12 ; [2H]C([2H])([2H])N1CC[C@]2(c3ccc(OC)c(OC)c3)C=C[C@@H](O)C[C@H]12 ; [2H]C([2H])([2H])N1CC[C@]2(c3ccc(OC)c(OC)c3)C=C[C@H](O)C[C@H]12 ; [2H]C([2H])([2H])N1CC[C@]2(c3ccc(OC)c(OC)c3)CC[C@@H](O)C=C12 ; [2H]C([2H])([2H])N1CC[C@]2(c3ccc(OC)c(OC)c3)CC[C@H](O)C=C12]

115. The compound of embodiment 114, wherein the compound has the absolute stereochemistry shown.

116. A compound of Formula (IA-c), (IB-c) or (IC-c):

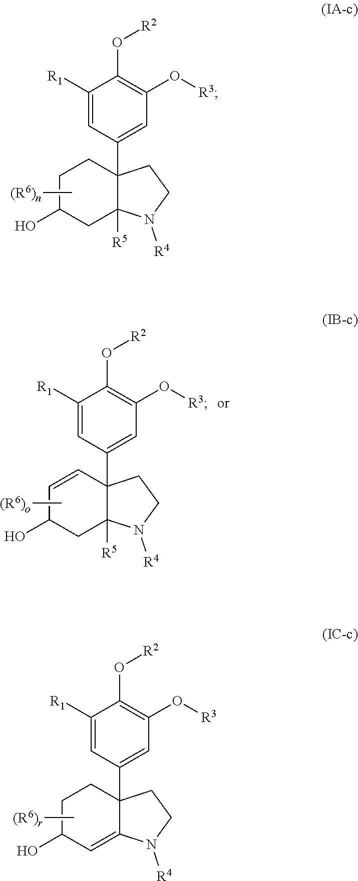

or a pharmaceutically acceptable salt thereof; wherein
R¹ is deuterium, halo, alkyl, alkenyl, alkynyl, cycloalkyl, cycloalkenyl, heterocyclyl, aryl, heteroaryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b};
wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R⁴ is H or alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a};
R⁵ is H, deuterium, or methyl;
each R⁶ is independently deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or two R⁶s on different carbon atoms together with the carbon atoms to which they are attached combine to form a cycloalkyl, heterocyclyl, or a heteroaryl, wherein each hydrogen atom in cycloalkyl, heterocyclyl, and heteroaryl is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or two R's on a single carbon atom together with the carbon atom to which they are attached combine to form cycloalkyl, heterocyclyl or heteroaryl, wherein each hydrogen atom in cycloalkyl, heterocyclyl, and 5 heteroaryl is optionally substituted by halo, hydroxy, alkoxy, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
n is 0, 1, 2, 3, 4, 5, or 6;
o is 0, 1, 2, 3, or 4;
r is 0, 1, 2, 3, 4, or 5;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl;
provided the compound is not:

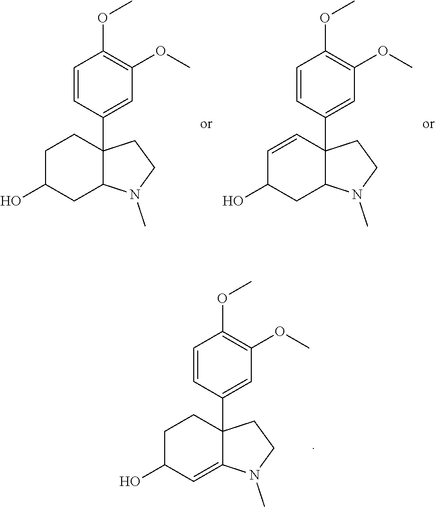 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

117. The compound of embodiment 116, wherein R⁴ is methyl optionally substituted with deuterium.

118. The compound of embodiment 117, wherein R⁴ is methyl.

119. The compound of embodiment 117, wherein R⁴ is —CD₃.

120. The compound of any one of embodiments 116-119, wherein R¹ is halo, alkyl, alkenyl, alkynyl, cycloalkyl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, and alkynyl, is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}.

121. The compound of embodiment 120, wherein R¹ is halo, alkyl, alkenyl, cycloalkyl, —OR^{a}, C(O)NR^{a}R^{b}, or —CN.

122. The compound of embodiment 121, wherein R¹ is fluoro.

123. The compound of embodiment 121, wherein R¹ is chloro.

124. The compound of embodiment 121, wherein R¹ is bromo.

125. The compound of embodiment 121, wherein R¹ is iodo.

126. The compound of embodiment 121, wherein R¹ is methoxy.

127. The compound of embodiment 121, wherein R¹ is acetyl.

128. The compound of embodiment 121, wherein R¹ is alkenyl.

129. The compound of embodiment 121, wherein R¹ is propenyl.

130. The compound of embodiment 121, wherein R¹ is cyclopropyl.

131. The compound of embodiment 121, wherein R¹ is methyl.

132. The compound of embodiment 121, wherein R¹ is ethyl.

133. The compound of embodiment 121, wherein R¹ is —CN.

134. The compound of embodiment 121, wherein R¹ is -amide.

135. The compound of embodiment 121, wherein R¹ is —C(O)—NH₂.

136. The compound of any one of embodiments 116-135, wherein each of R² and R³ is independently alkyl, cycloalkyl, alkenyl, or alkynyl; wherein each hydrogen atom in alkyl, cycloalkyl, alkenyl, and alkynyl, is optionally substituted by halo, deuterium, cycloalkyl, aryl, or OR^{a}; or R² and R³ together with the atoms to which they are attached combine to form heterocyclyl, wherein each hydrogen atom in heterocyclyl and heteroaryl is optionally substituted by halo or OR^{a}.

137. The compound of any one of embodiments 116-135, wherein each of R² and R³ is independently C₁-C₆ alkyl, C₃-C₆ cycloalkyl, C₂-C₆ alkenyl, or C₂-C₆ alkynyl; wherein each hydrogen atom in alkyl, cycloalkyl, alkenyl, and alkynyl, is optionally substituted by halo, deuterium, C₃-C₆ cycloalkyl, C₅-C₆ aryl, or OR^{a}; or R² and R³ together with the atoms to which they are attached combine to form a 4-6 membered heterocyclyl, wherein each hydrogen atom in heterocyclyl is optionally substituted by halo or OR^{a}.

138. The compound of embodiment 137, wherein R² and R³ are each independently C₁-C₆ alkyl optionally substituted with halo or deuterium.

139. The compound of embodiment 137, wherein R² and R³ are each independently methyl or ethyl optionally substituted with halo or deuterium.

140. The compound of embodiment 137, wherein R² and R³ are each independently methyl or ethyl optionally substituted with halo.

141. The compound of embodiment 137, wherein R² and R³ are each independently methyl or ethyl optionally substituted with fluoro.

142. The compound of embodiment 137, wherein R² and R³ are each independently methyl optionally substituted with fluoro.

143. The compound of embodiment 137, wherein R² is methyl and R³ is ethyl optionally substituted with fluoro.

144. The compound of embodiment 137, wherein R² is methyl and R³ is —CH₂CF₃ or —CHF₂.

145. The compound of embodiment 137, wherein R² and R³ are each independently —CHF₂.

146. The compound of embodiment 137, wherein R² and R³ are each independently methyl, —CH₂CHF₂, —CHF₂ or —CF₃.

147. The compound of embodiment 137, wherein R² and R³ are each independently —CHF₂.

148. The compound of embodiment 137, wherein R² is methyl and R³ is —CHF₂ or —CF₃.

149. The compound of embodiment 137, wherein R² is methyl and R³ is methyl.

150. The compound of embodiment 137, wherein R² and R³ are each independently ethyl optionally substituted with fluoro.

151. The compound of embodiment 137, wherein R² and R³ are each independently methyl or —CD₃.

152. The compound of embodiment 137, wherein R² is methyl and R³ is —CD₃.

153. The compound of embodiment 137, wherein R² is —CD₃ and R³ is methyl.

154. The compound of embodiment 137, wherein R² and R³ are each independently C₁-C₄ alkyl optionally substituted with C₃-C₆ cycloalkyl or C₂-C₄ alkenyl.

155. The compound of embodiment 137, wherein R² and R³ are each independently C₁-C₄ alkyl optionally substituted with unsubstituted cyclopropyl, cyclobutyl, or cyclohexyl.

156. The compound of embodiment 137, wherein R² is C₁-C₄ alkyl, and R³ is C₁-C₄ alkyl optionally substituted with cyclopropyl.

157. The compound of embodiment 140, wherein each of R² and R³ is independently C₃-C₆ cycloalkyl —CH₂-cyclopropyl, —CH₂-alkenyl, C₁-C₄ alkyl, or C₁-C₄ alkyl substituted with —ORₐ, and R^{a} is C₁-C₄ alkyl.

158. The compound of embodiment 137, wherein R² is —CH₂-cyclopropyl.

159. The compound of embodiment 158, wherein R³ is C₁-C₄ alkyl.

160. The compound of embodiment 137, wherein R³ is propylene.

161. The compound of embodiment 137, wherein R³ is cyclopentyl.

162. The compound of embodiment 137, wherein R³ is cyclohexyl.

163. The compound of embodiment 137, wherein R² is C₁-C₄ alkyl.

164. The compound of embodiment 163, wherein R³ is C₁-C₄ alkyl.

165. The compound of embodiment 158, wherein R³ is —CH₂-cyclopropyl.

166. The compound of embodiment 137, wherein each of R² and R³ is independently C₁-C₄ alkyl optionally substituted with —ORₐ, and Rₐ is C₁-C₄ alkyl.

167. The compound of embodiment 166, wherein R² is —CH₂—O—CH₃.

168. The compound of embodiment 167, wherein R³ is —CH₂—O—CH₃.

169. The compound of embodiment 137, wherein R³ is —CH₂—O—CH₃.

170. The compound of embodiment 137, wherein R³ is benzyl.

171. The compound of embodiment 170, wherein R² is C₁-C₄ alkyl.

172. The compound of any one of embodiments 116-135, wherein R² and R³ together with the atoms to which they are attached combine to form heterocyclyl, wherein each hydrogen atom in heterocyclyl is optionally substituted by halo or OR^{a}.

173. The compound of embodiment 172, wherein R² and R³ together with the atoms to which they are attached combine to form a 5-6 membered heterocyclyl.

174. The compound of embodiment 173, wherein R² and R³ together with the atoms to which they are attached combine to form a 5-6 membered heterocyclyl that is optionally substituted by halo or OR^{a} and R^{a} is C₁-C₄ alkyl.

175. The compound of embodiment 173, wherein R² and R³ together with the atoms to which they are attached combine to form a 5-6 membered heterocyclyl that is optionally substituted by fluoro.

176. The compound of embodiment 173, wherein R² and R³ together with the atoms to which they are attached combine to form a 5-6 membered heterocyclyl that is optionally substituted by methoxy.

177. The compound of embodiment 173, wherein R² and R³ are taken together with the oxygen atoms to which each is attached to form —O—CF₂—O—.

178. The compound of embodiment 173, wherein R² and R³ are taken together with the oxygen atoms to which each is attached to form a dioxolane optionally substituted by halo.

179. The compound of embodiment 173, wherein R² and R³ are taken together with the oxygen atoms to which each is attached to forma dioxolane optionally substituted by fluoro.

180. The compound of any one of embodiments 116-179, wherein R^{a} is methyl.

181. The compound of any one of embodiments 116-180, wherein R⁴ is H or C₁-C₆ alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a}.

182. The compound of embodiment 181, wherein R⁴ is C₁-C₄ alkyl.

183. The compound of embodiment 181, wherein R⁴ is methyl.

184. The compound of embodiment 181, wherein R⁴ is ethyl.

185. The compound of embodiment 181, wherein R⁴ is isopropyl.

186. The compound of embodiment 181, wherein R⁴ is —CH₂-cycloalkyl.

187. The compound of embodiment 181, wherein R⁴ is —CH₂-cyclopropyl.

188. The compound of embodiment 181, wherein R⁴ is benzyl optionally substituted by alkoxy.

189. The compound of embodiment 181, wherein R⁴ is benzyl optionally substituted by methoxy.

190. The compound of embodiment 181, wherein R⁴ is methyl wherein one or more hydrogen is substituted with deuterium.

191. The compound of embodiment 181, wherein R⁴ is methyl wherein one or more hydrogen is substituted with deuterium.

192. The compound of embodiment 181, wherein R⁴ is —CD₃.

193. The compound of any one of embodiments 116-192, wherein R⁵ is hydrogen.

194. The compound of any one of embodiments 116-192, wherein R⁵ is hydrogen.

195. The compound of embodiment 181, wherein R⁴ is C₁-C₆ alkyl, wherein each hydrogen atom in alkyl is optionally substituted by halo, deuterium, cycloalkyl, aryl, —OR^{a}, or —NR^{a}R^{b}; wherein each hydrogen atom in aryl is optionally substituted by OR^{a}.

196. The compound of any one of embodiments 116-195, wherein the compound is a compound of Formula (IA-d), (IB-d), or (IC-d):

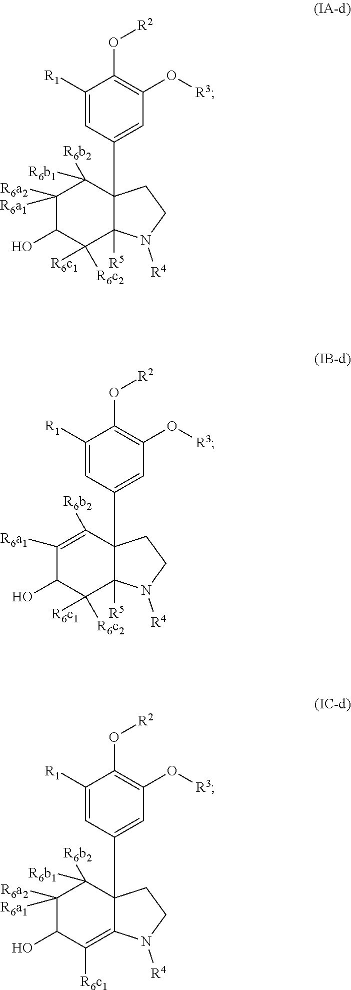

or a pharmaceutically acceptable salt thereof; wherein
each R₆ₐ₁, R₆ₐ₂, R_{6b1}, R_{6b2}, R_{6c1} and R_{6c2} is independently hydrogen, deuterium, alkyl, cycloalkyl, —Si(alkyl)₃, —C(O)alkyl, aryl, heteroaryl, or heterocyclyl, wherein each hydrogen atom in alkyl, cycloalkyl, —Si(alkyl)₃, —C(O)alkyl, phenyl, heteroaryl, and heterocyclyl is optionally substituted by halo, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form aryl, cycloalkyl, heteroaryl, or heterocyclyl;
or R₆ₐ₂ and R_{6b2} combine to form a cycloalkyl, or heterocyclyl, wherein each hydrogen atom in cycloalkyl and heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or R_{6b2} and R_{6c2} combine to form a cycloalkyl, or heterocyclyl, wherein each hydrogen atom in cycloalkyl, or heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or R_{6c1} and R_{6c2} combine to form a cycloalkyl, or heterocyclyl, wherein each hydrogen atom in cycloalkyl, or heterocyclyl, is optionally substituted by halo, hydroxy, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}.

197. The compound of embodiment 196, wherein R₆ₐ₂ and R_{6b2} combine to form an epoxide.

198. The compound of embodiment 197, wherein R₆ₐ₁ and R_{6b1} are each hydrogen.

199. The compound of embodiment 198, wherein R_{6c1} and R_{6c2} are each hydrogen.

200. The compound of any one of embodiments 196-199, wherein the compound is a compound of Formula (IA-d).

201. The compound of embodiment 196, wherein R_{6b2} and R_{6c2} combine to form a cycloalkyl or heterocyclyl, wherein each hydrogen atom in cycloalkyl and heterocyclyl, is optionally substituted by halo, hydroxy, alkyl, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}.

202. The compound of embodiment 201, wherein R_{6b2} and R_{6c2} combine to form a cycloalkyl.

203. The compound of embodiment 202, wherein R_{6b2} and R_{6c2} combine to form a bridging C₁-C₄ alkyl.

204. The compound of embodiment 203, wherein R_{6b2} and R_{6c2} combine to form a bridging methylene.

205. The compound of embodiment 204, wherein R_{6b2} and R_{6c2} combine to form a bridging ethylene.

206. The compound of embodiment 204, wherein R_{6b2} and R_{6c2} combine to form a bridging propylene.

207. The compound of any one of embodiments 201-206, wherein R₆ₐ₁, R₆ₐ₂, R_{6b1}, and R_{6c1} are each hydrogen.

208. The compound of any one of embodiments 201-207, wherein the compound is a compound of Formula (IA-d).

209. The compound of embodiment 196, wherein R_{6c1} and R_{6c2} combine to form a cycloalkyl or heterocyclyl, wherein each hydrogen atom in cycloalkyl and heterocyclyl, is optionally substituted by halo, hydroxy, alkyl, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b}.

210. The compound of embodiment 209, wherein R_{6c1} and R_{6c2} combine to form a cycloalkyl.

211. The compound of embodiment 209, wherein R_{6c1} and R_{6c2} combine to form a cyclopropyl.

212. The compound of any one of embodiments 209-211, wherein R₆ₐ₁, R₆ₐ₂, R_{6b1}, and R_{6b2} are each hydrogen.

213. The compound of embodiment 196, wherein each R₆ₐ₁, R₆ₐ₂, R_{6b1}, R_{6b2}, R_{6c1} and R_{6c2} is independently hydrogen, deuterium, or methyl optionally substituted with deuterium or halo.

214. The compound of embodiment 196, wherein each of R₆ₐ₁, R₆ₐ₂, R_{6b1}, R_{6b2}, R_{6c1} and R_{6c2} is hydrogen or methyl, provided that only one of R₆ₐ₁, R₆ₐ₂, R_{6b1}, R_{6b2}, R_{6c1} and R_{6c2} is methyl.

215. The compound of embodiment 196, wherein R_{6b1} is methyl and R_{6b2} is hydrogen.

216. The compound of embodiment 215, wherein each of R₆ₐ₁, R₆ₐ₂, R_{6c1}, and R_{6c2} is hydrogen.

217. The compound of embodiment 196, wherein R₆ₐ₁ is —CD₃ and R₆ₐ₂ is hydrogen.

218. The compound of embodiment 196, wherein R₆ₐ₁ is —CF₃ and R₆ₐ₂ is hydrogen.

219. The compound of embodiment 196, wherein R₆ₐ₁ is alkyl optionally substituted with halo and R₆ₐ₂ is hydrogen.

220. The compound of embodiment 196, wherein R₆ₐ₁ is methyl optionally substituted with fluoro and R₆ₐ₂ is hydrogen.

221. The compound of any one of embodiments 217-220, wherein R_{6b1}, R_{6b2}, R_{6c1} and R_{6c2} are each hydrogen.

222. The compound of any one of embodiments 196-199, or 201-221, wherein the compound is a compound of Formula (IA-d).

223. The compound of any one of embodiments 196-199 or 201-221, wherein the compound is a compound of Formula (IB-d).

224. The compound of any one of embodiments 196-199 or 201-221, wherein the compound is a compound of Formula (IC-d).

225. A pharmaceutical composition, comprising a compound according to any one of embodiments 1-224; and a pharmaceutical acceptable excipient.

226. A method of treating a mental health disorder, comprising administering to a mammal in need thereof an effective amount of a compound according to any one of embodiments 1-224 or a pharmaceutically acceptable salt thereof.

227. The method of embodiment 226, wherein the mental health disorder is anxiety, stress, or depression.

228 The method of embodiment 226, wherein the mental health disorder is anxiety.

229. The method of embodiment 226, wherein the mental health disorder is stress.

230. The method of embodiment 226, wherein the mental health disorder is depression.

231. The method of any one of embodiments 226-230, wherein the mammal is a human.

232. A method of treating an inflammatory condition, comprising administering to a mammal in need thereof an effective amount of a compound according to any one of embodiments 1-224 or a pharmaceutically acceptable salt thereof.

233. The method of embodiment 232, wherein the inflammatory condition is chronic obstructive pulmonary disease (COPD), asthma, or rheumatoid arthritis.

234. The method of embodiment 232, wherein the inflammatory condition is COPD.

235. The method of embodiment 232, wherein the inflammatory condition is asthma.

236. The method of embodiment 232, wherein the inflammatory condition is rheumatoid arthritis.

237. The method of any one of embodiments 232-236, wherein the mammal is a human.

In some embodiments, the compound is a compound of one or more of the following additional embodiments:

A-1. A compound of Formula (IA), (IB) or (IC):

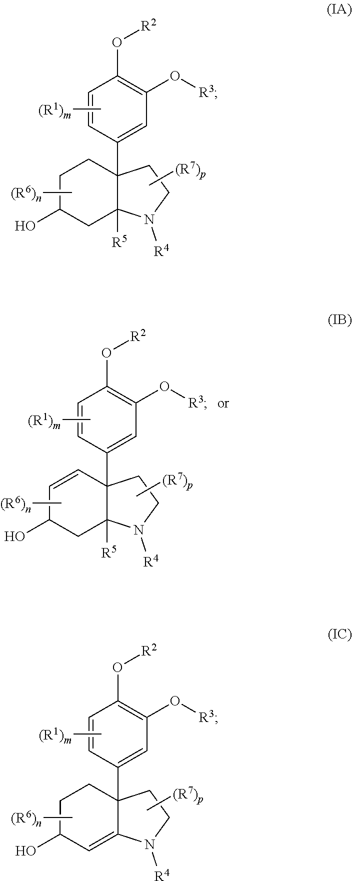

or a pharmaceutically acceptable salt thereof; wherein
each R¹ is independently halo, deuterium, C₁₋₄ alkyl, C₂₋₄ alkenyl, C₂₋₄ alkynyl, C₃₋₈ cycloalkyl, C₃₋₈ cycloalkenyl, 3-8 membered heterocyclyl comprising one or more N, O or S heteroatoms, C₅₋₆ aryl, 5-6 membered heteroaryl comprising one or more N, O or S heteroatoms, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, and alkynyl, is optionally substituted by halo, C₁₋₄ alkyl, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; and wherein each hydrogen atom in cycloalkyl, cycloalkenyl, heterocyclyl, aryl, and heteroaryl is optionally substituted by halo, C₁₋₄ alkyl, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently C₁₋₄ alkyl, H, C₃₋₈ cycloalkyl, C₂₋₄ alkenyl, C₂₋₄ alkynyl, or C₅₋₆ aryl; wherein each hydrogen atom in alkyl, alkenyl, and alkynyl, is optionally substituted by halo, deuterium, C₃₋₈ cycloalkyl, C₅₋₆ aryl, or OR^{a}; and wherein each hydrogen atom in cycloalkyl, and aryl is optionally substituted by halo, deuterium, C₁₋₄ alkyl, C₁₋₄ haloalkyl or OR^{a}; or
R² and R³ together with the atoms to which they are attached combine to form 3-8 membered heterocyclyl comprising one or more N, O or S heteroatoms, or 5-6 membered heteroaryl comprising one or more N, O or S heteroatoms, wherein each hydrogen atom in heterocyclyl and heteroaryl is optionally substituted by halo, C₁₋₄ alkyl, C₁₋₄ haloalkyl or OR^{a};
R⁴ is H or C₁₋₄ alkyl, wherein each hydrogen atom in alkyl is optionally substituted by halo, deuterium, C₃₋₈ cycloalkyl, C₅₋₆ aryl, —OR^{a}, or —NR^{a}R^{b}; wherein each hydrogen atom in aryl is optionally substituted by OR^{a};
R⁵ is H, deuterium, or methyl;
each R⁶ is independently C₁₋₄ alkyl, deuterium, C₃₋₈ cycloalkyl, —Si(C₁₋₄ alkyl)₃, —C(O)—(C₁₋₄)-alkyl, C₅₋₆ aryl, 5-6 membered heteroaryl comprising one or more N, O or S heteroatoms, or 3-8 membered heterocyclyl comprising one or more N, O or S heteroatoms; wherein each hydrogen atom in alkyl, —Si(alkyl)₃, and —C(O)alkyl is optionally substituted by halo, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; and wherein each hydrogen atom in cycloalkyl, phenyl, heteroaryl, and heterocyclyl is optionally substituted by halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; or two substituents on alkyl, cycloalkyl, —Si(alkyl)₃, —C(O)alkyl, phenyl, heteroaryl, and heterocyclyl together with the atoms to which they are attached combine to form C₅₋₆ aryl, C₃₋₈ cycloalkyl, 5-6 membered heteroaryl comprising one or more N, O or S heteroatoms, or 3-8 membered heterocyclyl comprising one or more N, O or S heteroatoms;
or two R⁶s on different carbon atoms together with the carbon atoms to which they are attached combine to form a C₃₋₈ cycloalkyl, 3-8 membered heterocyclyl comprising one or more N, O or S heteroatoms, or a 5-6 membered heteroaryl comprising one or more N, O or S heteroatoms, wherein each hydrogen atom in cycloalkyl, heterocyclyl, and heteroaryl is optionally substituted by halo, hydroxy, C₁₋₄ alkyl optionally substituted by one or more halo, C₁₋₄ alkanol, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form C₃₋₈ cycloalkyl, 3-8 membered heterocyclyl comprising one or more N, O or S heteroatoms, or 5-6 membered heteroaryl comprising one or more N, O or S heteroatoms, wherein each hydrogen atom in cycloalkyl, heterocyclyl, and 5-6 membered heteroaryl comprising one or more N, O or S heteroatoms is optionally substituted by halo, hydroxy, C₁₋₄ alkoxy, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, C₁₋₄ alkyl, C₁₋₄ haloalkyl, —OH, or —NH₂; wherein each hydrogen atom in alkyl is optionally substituted by —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
each R^{a} and R^{b} is independently H, C₁₋₄ alkyl, C₂₋₄ alkenyl, C₃₋₈ cycloalkyl, 3-8 membered heterocyclyl comprising one or more N, O or S heteroatoms, C₅₋₆ aryl, or 5-6 membered heteroaryl;
or if an instance of R¹ is —NR^{a}R^{b}, then R^{a} and R^{b} may combine with the nitrogen atom to which they are attached to form 3-8 membered heterocyclyl or 5-6 membered heteroaryl; wherein each hydrogen atom in alkyl, or alkenyl, is optionally substituted by halo, hydroxy, C₁₋₄ alkyl, C₁₋₄ alkanol, C₅₋₆ aryl, —OR^{c}, —NR^{c}R^{d}, —CHO, —C(O)R^{c}, —CO₂R^{c}, —C(O)NR^{c}R^{d}, —CN, nitro, or —P(O)OR^{c}R^{d}; wherein each hydrogen atom in cycloalkyl, heterocyclyl, aryl, or heteroaryl is optionally substituted by halo, hydroxy, C₁₋₄ alkyl, C₁₋₄ haloalkyl, C₁₋₄ alkanol, —OR^{c}, —NR^{c}R^{d}, —CHO, —C(O)R^{c}, —CO₂R^{c}, —C(O)NR^{c}R^{d}, —CN, nitro, or —P(O)OR^{c}R^{d}; and
each R^{c} and R^{d} is independently H, C₁₋₄ alkyl, C₂₋₄ alkenyl, C₃₋₈ cycloalkyl, 3-8 membered heterocyclyl comprising one or more N, O or S heteroatoms, C₅₋₆ aryl, or 5-6 membered heteroaryl comprising one or more N, O or S heteroatoms;
provided the compound is not:

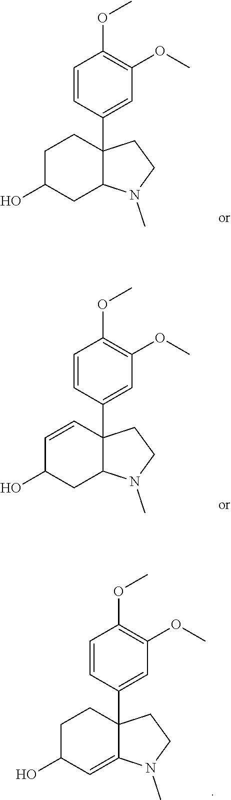 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

A-2. The compound of embodiment A-1, wherein p is 0.

A-3. The compound of embodiment A-2, wherein R⁵ is H.

A-4. The compound of embodiment A-3, wherein R⁴ is C₁₋₄ alkyl.

A-5. The compound of embodiment A-4, wherein each R^{a} and R^{b} is independently H, or C₁₋₄ alkyl.

A-6. The compound of embodiment A-5, herein the compound is a compound of Formula (IA-c), (IB-c) and (IC-c)

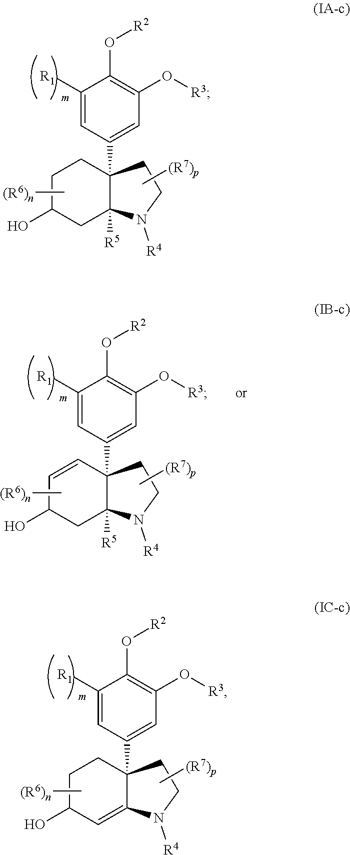

wherein m is 0 or 1; and n is 0, 1, or 2; or a pharmaceutically acceptable salt thereof.

A-7. The compound of embodiment A-6, wherein each R¹ is independently halo, C₁₋₄ alkyl, C₁₋₄ haloalkyl, C₂₋₄ alkenyl, —OR^{a}, —NR^{a}R^{b}, —CHO, —C(O)R^{a}, —CO₂R^{a}, —C(O)NR^{a}R^{b}, —CN, or C₃₋₆ cycloalkyl.

A-8. The compound of embodiment A-7, wherein n is 0, 1 or 2.

A-9. The compound of embodiment A-8, wherein
a. n is 1 or 2 and each R⁶ is independently C₁₋₄ alkyl; or
b. n is 2 and two R⁶s on a single carbon atom together with the carbon atom to which they are attached combine to form a spirocyclic C₃₋₆ cycloalkyl or a 3-6 membered heterocyclyl comprising one or more N, O or S heteroatoms; or
c. n is 2 and two R⁶s on different carbon atoms together with the carbon atoms to which they are attached combine to form a C₅₋₈ cycloalkyl, 5-8 membered heterocyclyl comprising one or more N, O or S heteroatoms.

A-10. The compound of embodiment A-9, wherein
a. each R¹ is independently halo, C₁₋₄ alkyl, C₁₋₄ haloalkyl, —CH═CH₂, —OCH₃, —NR^{a}R^{b}, —CHO, —C(O)R^{a}, —CO₂R^{a}, —C(O)NR^{a}R^{b}, —CN, or cyclopropyl; and R^{a} and R^{b} are each independently H or C₁₋₄ alkyl; and
b. each R⁶ is methyl; or two R⁶s on a single carbon atom together with the carbon atom to which they are attached combine to form a spirocyclic cyclopropyl or epoxy; or two R⁶s on different carbon atoms together with the carbon atoms to which they are attached combine to form a C₅₋₈ cycloalkyl.

A-11. The compound of embodiment A-10, wherein
a. each of R² and R³ is independently H, C₂₋₄ alkenyl, C₂₋₄ alkynyl, C₃₋₈ cycloalkyl, or C₁₋₄ alkyl, C₁₋₄ haloalkyl, OR^{a}, C₃₋₈ cycloalkyl, or phenyl; or
b. R² and R³ together with the atoms to which they are attached combine to form a 5-6 membered heterocyclyl comprising one or more N, O or S heteroatoms wherein each hydrogen atom in heterocyclyl is optionally substituted by halo, C₁₋₄ alkyl, C₁₋₄ haloalkyl or OR^{a}.

A-12. The compound of embodiment A-11, wherein R^{a} and R^{b} are each independently H or methyl.

A-13. The compound of embodiment A-12, wherein each of R² and R³ is independently H, —CH₂CH═CH₂, —CH₂(CCH), C₃₋₆ cycloalkyl, —(CH₂)—(C₃₋₆ cycloalkyl), or C₁₋₄ alkyl optionally substituted with one or more F, or methoxy.

A-14. The compound of embodiment A-12, wherein R² and R³ together with the atoms to which they are attached combine to form 3-6 membered heterocyclyl comprising two O heteroatoms.

A-15. The compound of embodiment A-12, wherein R² and R³ together with the atoms to which they are attached combine to form 5-6 membered heterocyclyl comprising two O heteroatoms, optionally substituted with one or more F.

A-16. The compound of embodiment A-1 wherein the compound is a compound of Formula (IX-A), (IX—B) or (IX—C)

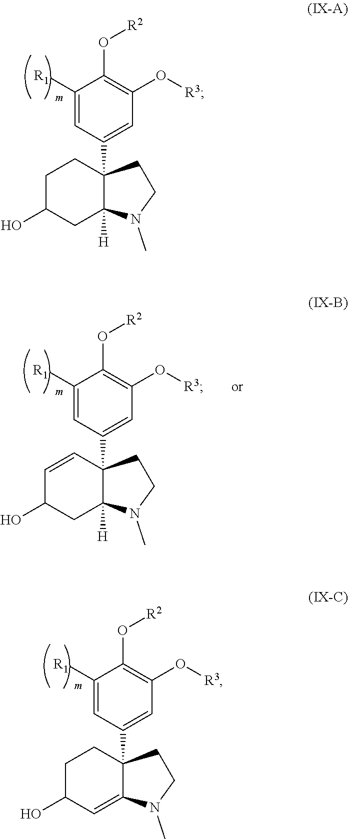

pharmaceutically acceptable salt thereof; wherein
R¹ is halo, methoxy, cyclopropyl, amido or acetyl;
m is 0 or 1;
each of R² and R³ is independently H, C₂₋₄ alkenyl, C₂₋₄ alkynyl, C₃₋₈ cycloalkyl, C₁₋₄ alkyl, C₁₋₄ haloalkyl, OR^{a}, C₃₋₈ cycloalkyl, or phenyl; or
R² an R³ together with the atoms to which they are attached combine to form a 5-6 membered heterocyclyl comprising one or more N, O or S heteroatoms wherein each hydrogen atom in the heterocyclyl is optionally substituted by halo, C₁₋₄ alkyl, C₁₋₄ haloalkyl or OR^{a}; and
each R^{a} is independently H or C₁₋₄ alkyl.

A-17. The compound of embodiment A-16, wherein each of R² and R³ is independently H, or C₁₋₄ alkyl optionally substituted with one or more F.

A-18. The compound of embodiment A-17, wherein each of R² and R³ is independently C₁₋₄ alkyl optionally substituted with one or more F.

A-19. The compound of embodiment A-18, wherein each of R^{a}, R² and R³ is independently methyl.

A-20. The compound of embodiment A-16, wherein R² an R³ together with the atoms to which they are attached combine to form a 5-6 membered heterocyclyl comprising two oxygen heteroatoms wherein each hydrogen atom in the heterocyclyl is optionally substituted by halo, C₁₋₄ alkyl, C₁₋₄ haloalkyl or OR^{a}.

A-21. The compound of embodiment A-16, wherein the compound is a compound of Formula (X-A), (X—B) or (X—C)

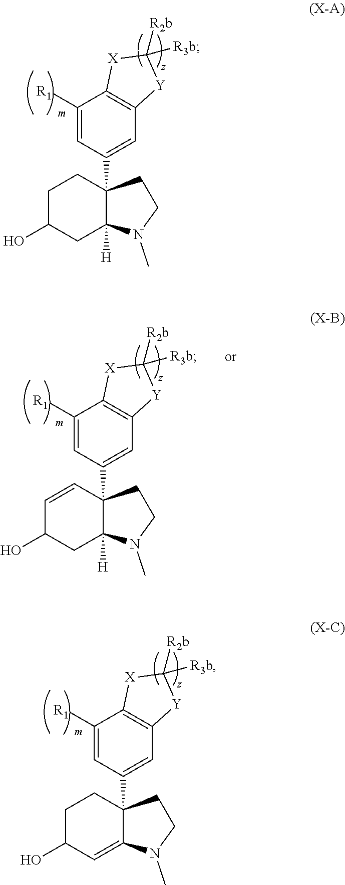

pharmaceutically acceptable salt thereof; wherein
X and Y are each O, NR^{a} or S;
R¹ is halo, methoxy, cyclopropyl, amido or acetyl;
m is 0 or 1;
z is 1 or 2;
each R^{2b} and R^{3b} is independently H, halo, C₁₋₄ alkyl, C₁₋₄ haloalkyl, OR^{a}, and C₃₋₈ cycloalkyl; and
each R^{a} is independently H or C₁₋₄ alkyl.

A-22. The compound of embodiment A-21, wherein X and Y are O.

A-23. The compound of embodiment A-22, wherein z is 1.

A-24. The compound of embodiment A-23, wherein each of R^{2b} and R^{3b} is independently H or F.

A-25. The compound of embodiment A-1, wherein the compound is a compound of Formula (VIII):

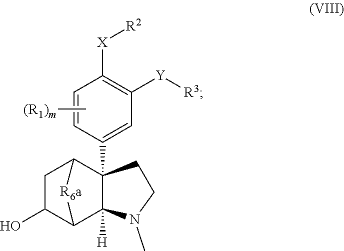

or a pharmaceutically acceptable salt thereof; wherein
X and Y are each independently O or S;
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, alkyl, cycloalkyl, alkenyl, alkynyl, or aryl; wherein each hydrogen atom in alkyl cycloalkyl, alkenyl, alkynyl, and aryl is optionally substituted by halo, deuterium, cycloalkyl, aryl, or OR^{a}; or R² and R³ together with the atoms to which they are attached combine to form heterocyclyl or heteroaryl, wherein each hydrogen atom in heterocyclyl and heteroaryl is optionally substituted by halo or OR^{a};
R₆ₐ is C₁₋₄ alkyl optionally comprising one or more N, S or O heteroatoms and forming a bridging cycloalkyl or heteroalkyl comprising a N, S, or O heteroatom to form a bridging heterocyclyl, wherein each hydrogen atom in cycloalkyl, or heterocyclyl, is optionally substituted by halo, hydroxy, alkyl, alkanol, aryl, —OR^{a}, —N^{a}R^{b}, —CHO, —C(O)R^{a}, —CO₂R^{a}, —C(O)NR^{a}R^{b}, —CN, nitro, or —P(O)OR^{a}OR^{b};
m is 0, 1, 2, or 3;
each R^{a} and R^{b} is independently H, alkyl, alkenyl, cycloalkyl, heterocyclyl, aryl, or heteroaryl; or if an instance of R¹ is —NR^{a}R^{b}, then R^{a} and R^{b} may combine with the nitrogen atom to which they are attached to form heterocyclyl or heteroaryl, wherein each hydrogen atom in alkyl, alkenyl, cycloalkyl, heterocyclyl, aryl, or heteroaryl is optionally substituted by halo hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

A-26. The compound of embodiment A-25, wherein
each R¹ is independently halo, C₂₋₄ alkenyl, —OR^{a}, —NR^{a}R^{b}, —CHO, —C(O)R^{a}, —CO₂R^{a}, —C(O)NR^{a}R^{b}, —CN, C₃₋₆ cycloalkyl, C₁₋₄ alkyl, or C₁₋₄ haloalkyl;
each of R² and R³ is independently H, C₂₋₄ alkenyl, C₂₋₄ alkynyl, C₃₋₈ cycloalkyl, C₁₋₄ alkyl, C₁₋₄ haloalkyl, OR^{a}, C₃₋₈ cycloalkyl, or phenyl; and
R₆ₐ is C₁₋₄ alkyl.

A-27. The compound of embodiment A-26, wherein each of R² and R³ is independently H or C₁₋₄ alkyl.

A-28. The compound of embodiment A-27, wherein each of R² and R³ is independently methyl.

A-29. The compound of embodiment A-28, wherein m is 0 or 1.

A-30. The compound of embodiment A-29, wherein m is 0.

A-31. The compound of any one of claims 1-5, wherein the compound is a compound of formula (IA).

A-32. The compound of any one of claims 1-5, wherein the compound is a compound of formula (IA-a)

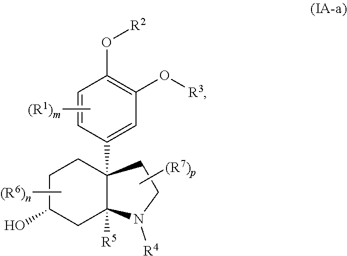

or a pharmaceutically acceptable salt thereof.

A-33. The compound of any one of claims 1-5, wherein the compound is a compound of formula (IA-b)

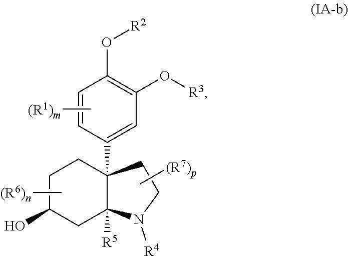

or a pharmaceutically acceptable salt thereof.

A-34. The compound of any one of claims 1-5, wherein the compound is a compound of formula (IB).

A-35. The compound of any one of claims 1-5, wherein the compound is a compound of formula (IB-a)

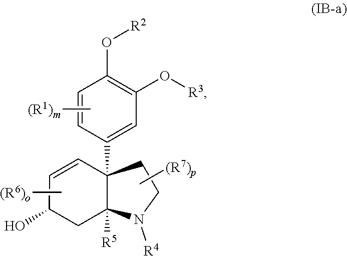

or a pharmaceutically acceptable salt thereof.

A-36. The compound of any one of claims 1-5, wherein the compound is a compound of formula (IB-b)

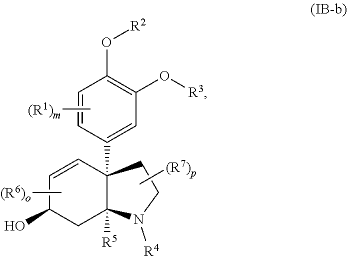

or a pharmaceutically acceptable salt thereof.

A-37. The compound of any one of claims 1-5, wherein the compound is a compound of formula (IC).

A-38. The compound of any one of claims 1-5, wherein the compound is a compound of formula (IC-a)

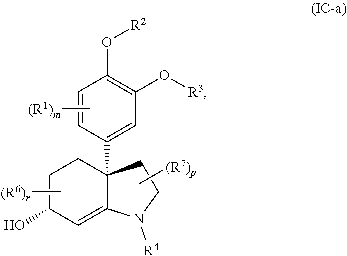

or a pharmaceutically acceptable salt thereof.

A-39. The compound of any one of claims 1-5, wherein the compound is a compound of formula (IC-b)

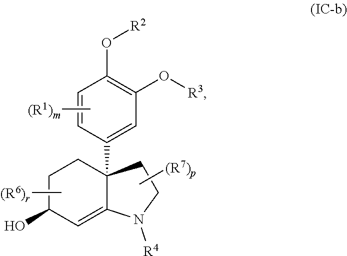

or a pharmaceutically acceptable salt thereof.

A-40. The compound of any one of claims 6-15, wherein the compound is a compound of formula (IA-c), or a pharmaceutically acceptable salt thereof.

A-41. The compound of any one of claims 6-15, wherein the compound is a compound of formula (IB-c), or a pharmaceutically acceptable salt thereof.

A-42. The compound of any one of claims 6-15, wherein the compound is a compound of formula (IC-c), or a pharmaceutically acceptable salt thereof.

A-43. The compound of any one of claims 16-20, wherein the compound is a compound of formula (IX-A), or a pharmaceutically acceptable salt thereof.

A-44. The compound of any one of claims 16-20, wherein the compound is a compound of formula (IX-A-a)

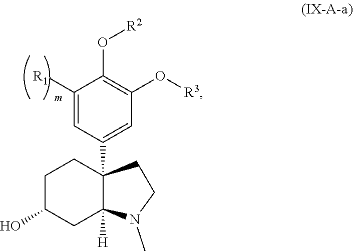

or a pharmaceutically acceptable salt thereof.

A-45. The compound of any one of claims 16-20, wherein the compound is a compound of formula (IX-A-b),

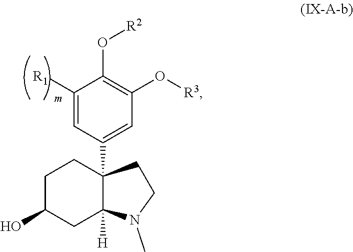

or a pharmaceutically acceptable salt thereof.

A-46. The compound of any one of claims 16-20, wherein the compound is a compound of formula (IX—B), or a pharmaceutically acceptable salt thereof.

A-47. The compound of any one of claims 16-20, wherein the compound is a compound of formula (IX—B-a),

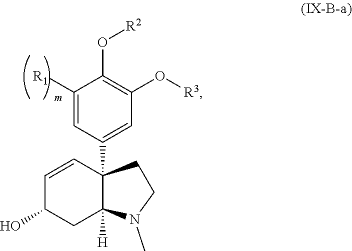

or a pharmaceutically acceptable salt thereof.

A-48. The compound of any one of claims 16-20, wherein the compound is a compound of formula (IX—B-b),

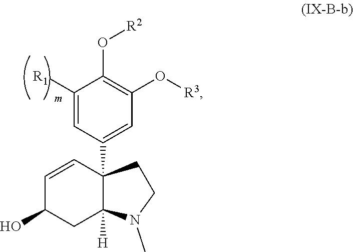

or a pharmaceutically acceptable salt thereof.

A-49. The compound of any one of claims 16-20, wherein the compound is a compound of formula (IX—C), or a pharmaceutically acceptable salt thereof.

A-50. The compound of any one of claims 16-20, wherein the compound is a compound of formula (IX—C-a)

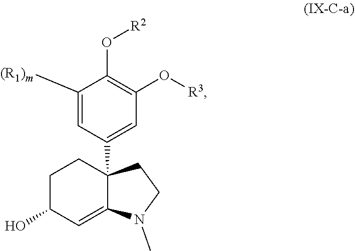

or a pharmaceutically acceptable salt thereof.

A-51. The compound of any one of claims 16-20, wherein the compound is a compound of formula (IX—C-b)

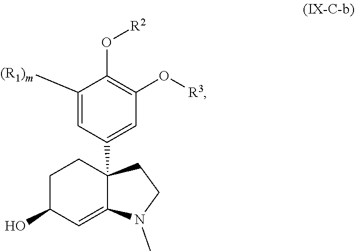

or a pharmaceutically acceptable salt thereof.

A-52. The compound of any one of claims 21-24, wherein the compound is a compound of formula (X-A).

A-53. The compound of any one of claims 21-24, wherein the compound is a compound of formula (X-A-a)

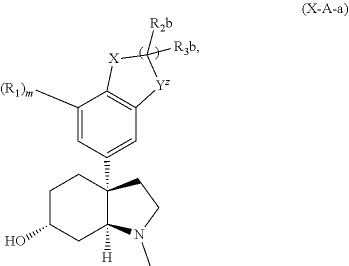

or a pharmaceutically acceptable salt thereof.

A-54. The compound of any one of claims 21-24, wherein the compound is a compound of formula (X-A-b)

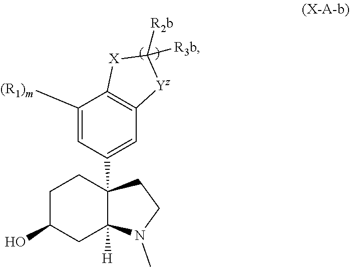

or a pharmaceutically acceptable salt thereof.

A-55. The compound of any one of claims 21-24, wherein the compound is a compound of formula (X—B), or a pharmaceutically acceptable salt thereof.

A-56. The compound of any one of claims 21-24, wherein the compound is a compound of formula (X—B-a)

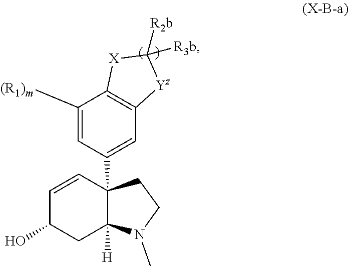

or a pharmaceutically acceptable salt thereof.

A-57. The compound of any one of claims 21-24, wherein the compound is a compound of formula (X—B-b)

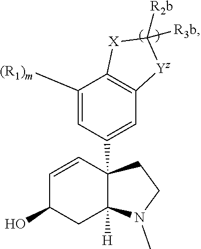

or a pharmaceutically acceptable salt thereof.

A-58. The compound of any one of claims 21-24, wherein the compound is a compound of formula (X—C), or a pharmaceutically acceptable salt thereof.

A-59. The compound of any one of claims 21-24, wherein the compound is a compound of formula (X—C-a)

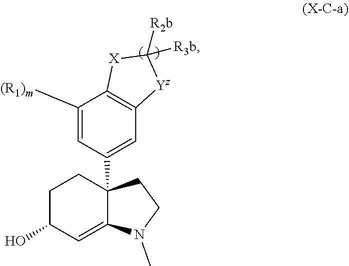

or a pharmaceutically acceptable salt thereof.

A-60. The compound of any one of claims 21-24, wherein the compound is a compound of formula (X—C-b)

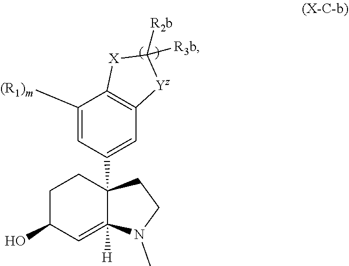

or a pharmaceutically acceptable salt thereof.

A-61. The compound of embodiment A-1, selected from:

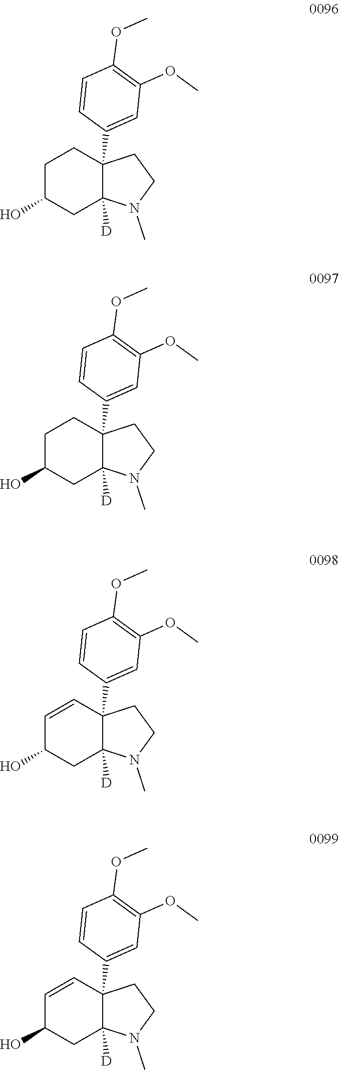 [SMILES: [2H][C@]12C[C@H](O)CC[C@@]1(c1ccc(OC)c(OC)c1)CCN2C ; [2H][C@]12C[C@@H](O)CC[C@@]1(c1ccc(OC)c(OC)c1)CCN2C ; [2H][C@]12C[C@H](O)C=C[C@@]1(c1ccc(OC)c(OC)c1)CCN2C ; [2H][C@]12C[C@@H](O)C=C[C@@]1(c1ccc(OC)c(OC)c1)CCN2C]

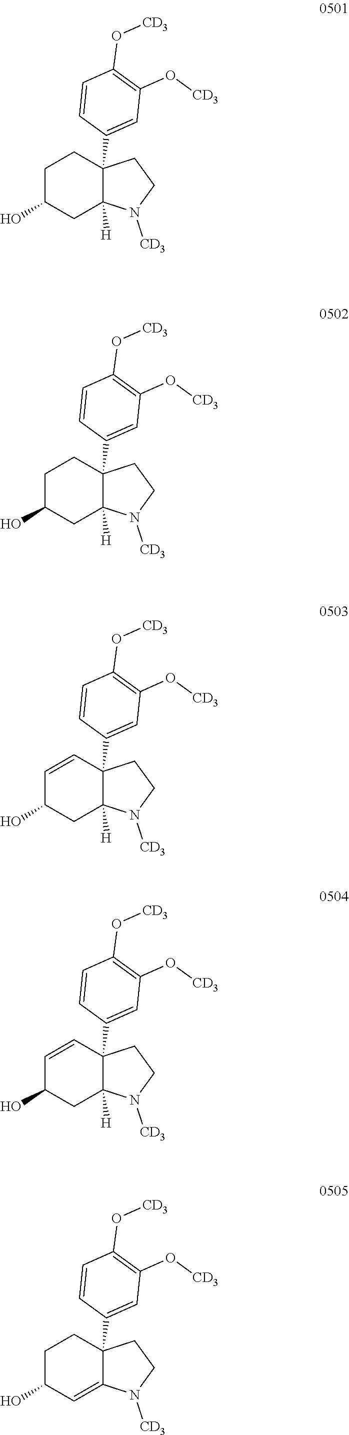 [SMILES: [2H]C([2H])([2H])Oc1ccc([C@@]23CC[C@@H](O)C[C@@H]2N(C([2H])([2H])[2H])CC3)cc1OC([2H])([2H])[2H] ; [2H]C([2H])([2H])Oc1ccc([C@@]23CC[C@H](O)C[C@@H]2N(C([2H])([2H])[2H])CC3)cc1OC([2H])([2H])[2H] ; [2H]C([2H])([2H])Oc1ccc([C@@]23C=C[C@@H](O)C[C@@H]2N(C([2H])([2H])[2H])CC3)cc1OC([2H])([2H])[2H] ; [2H]C([2H])([2H])Oc1ccc([C@@]23C=C[C@H](O)C[C@@H]2N(C([2H])([2H])[2H])CC3)cc1OC([2H])([2H])[2H] ; [2H]C([2H])([2H])Oc1ccc([C@@]23CC[C@@H](O)C=C2N(C([2H])([2H])[2H])CC3)cc1OC([2H])([2H])[2H]]

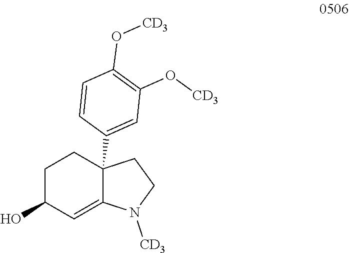 [SMILES: [2H]C([2H])([2H])Oc1ccc([C@@]23CC[C@H](O)C=C2N(C([2H])([2H])[2H])CC3)cc1OC([2H])([2H])[2H]]

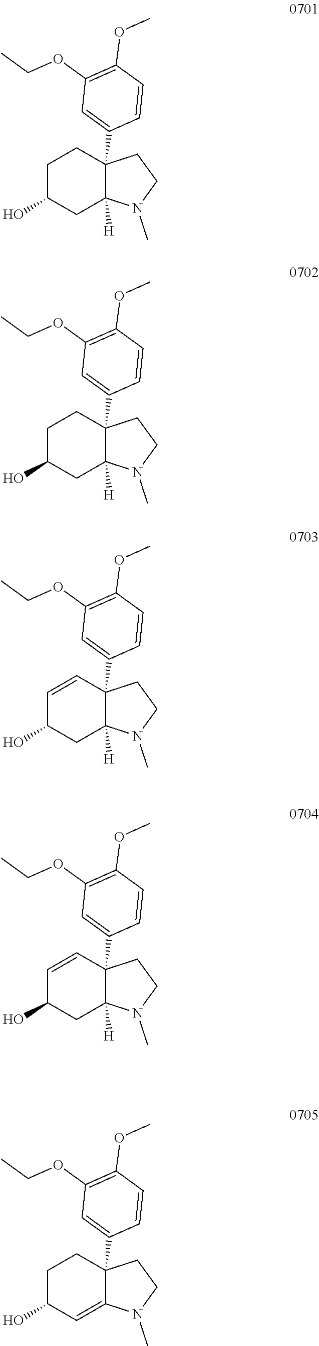

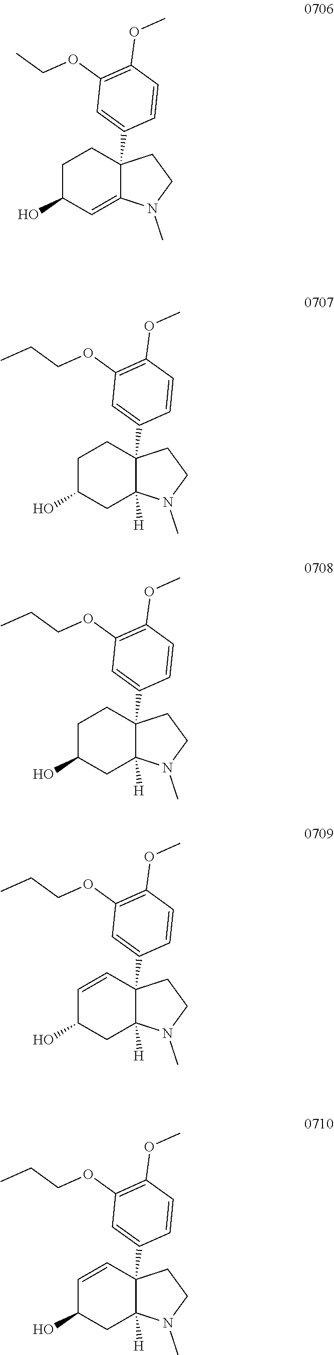

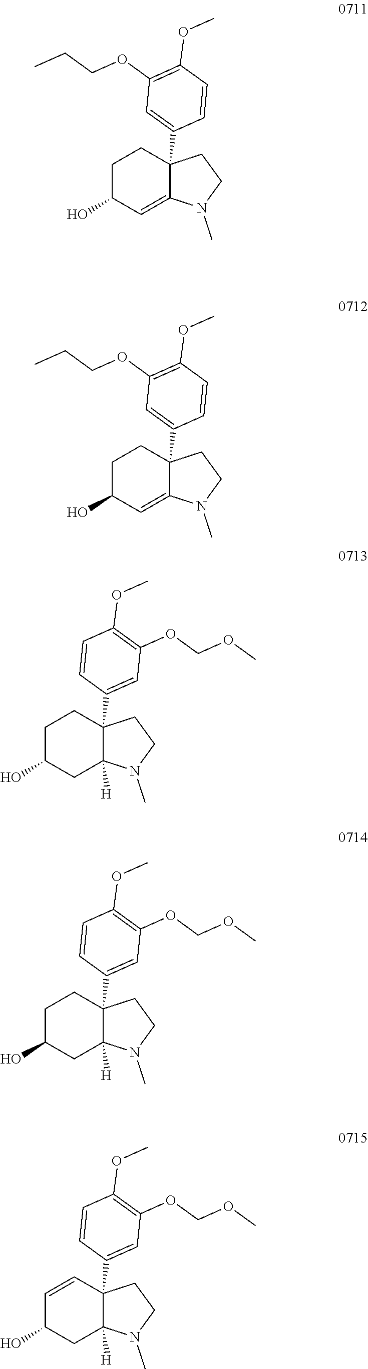 [SMILES: CCCOc1cc([C@@]23CC[C@@H](O)C=C2N(C)CC3)ccc1OC ; CCCOc1cc([C@@]23CC[C@H](O)C=C2N(C)CC3)ccc1OC ; COCOc1cc([C@@]23CC[C@@H](O)C[C@@H]2N(C)CC3)ccc1OC ; COCOc1cc([C@@]23CC[C@H](O)C[C@@H]2N(C)CC3)ccc1OC ; COCOc1cc([C@@]23C=C[C@@H](O)C[C@@H]2N(C)CC3)ccc1OC]

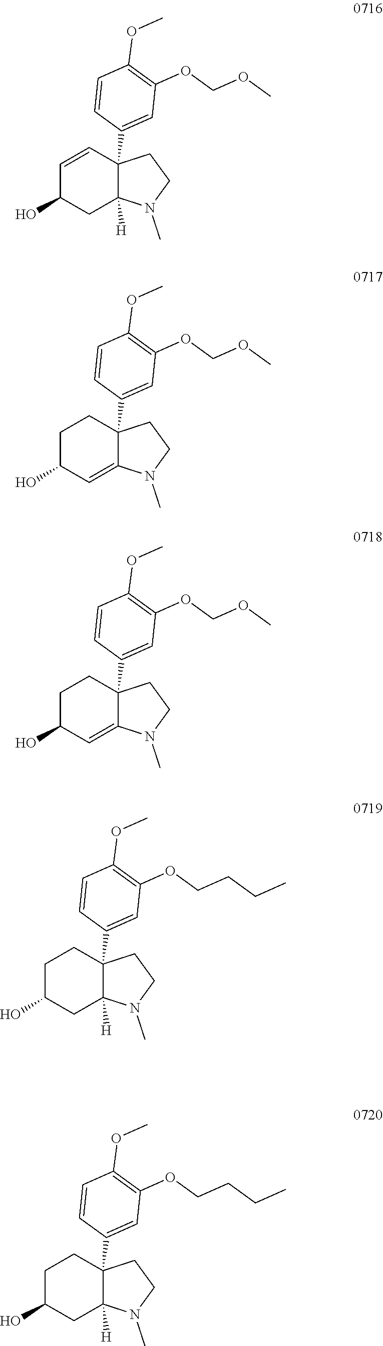

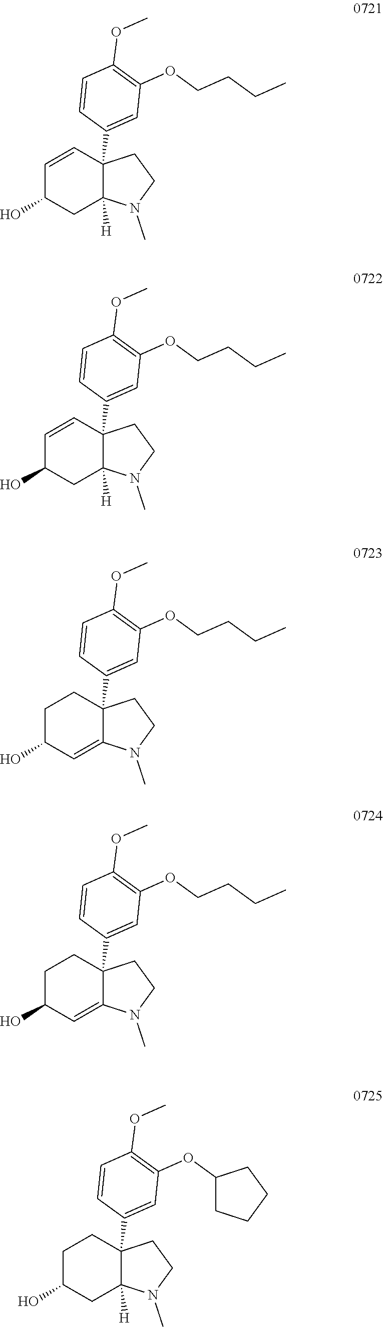

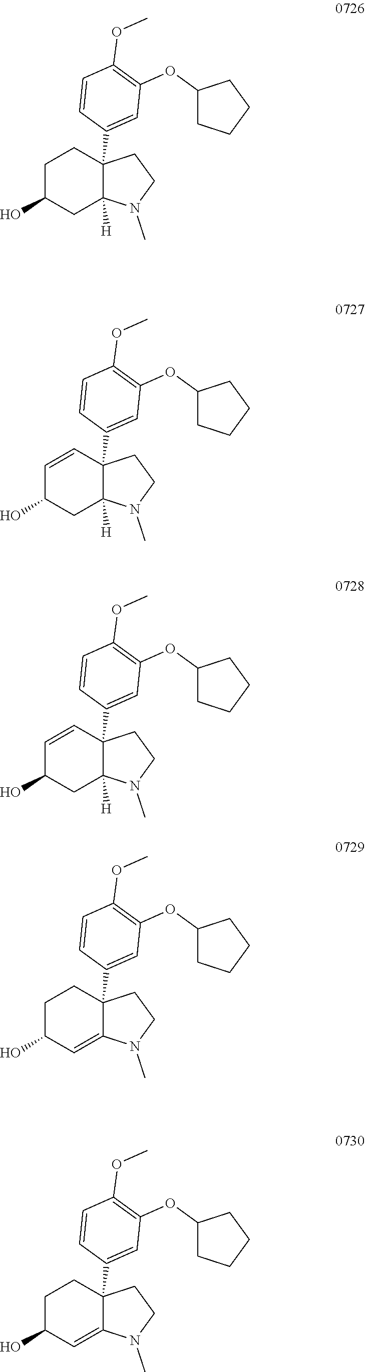

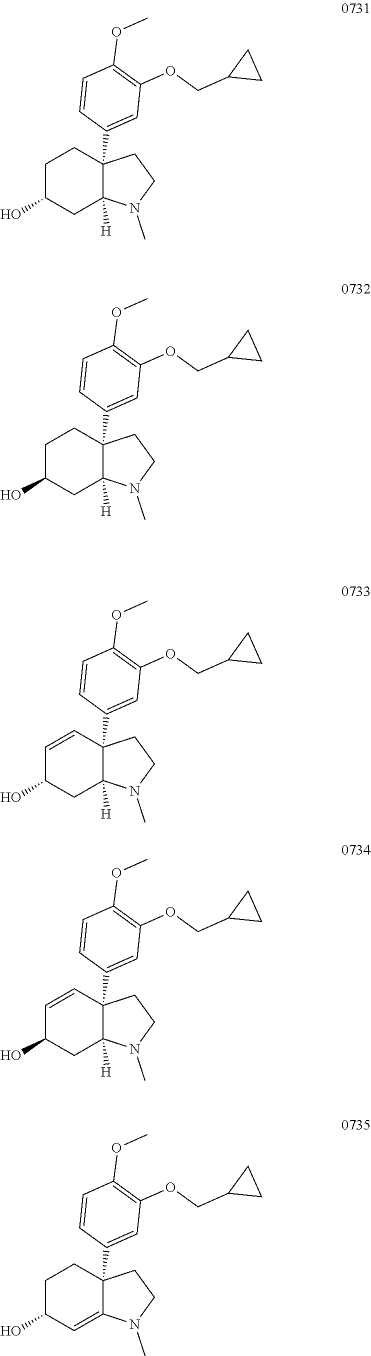

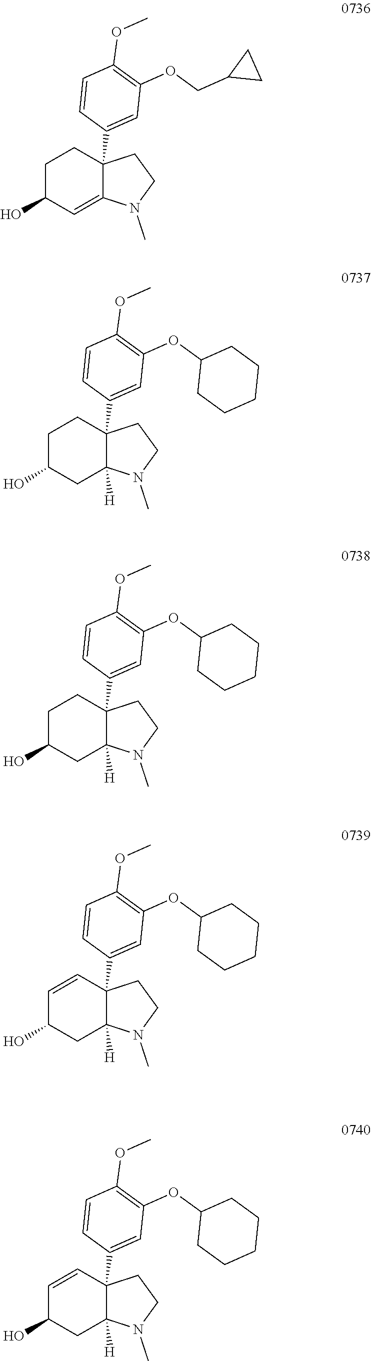 [SMILES: COc1ccc([C@@]23CC[C@H](O)C=C2N(C)CC3)cc1OCC1CC1 ; COc1ccc([C@@]23CC[C@@H](O)C[C@@H]2N(C)CC3)cc1OC1CCCCC1 ; COc1ccc([C@@]23CC[C@H](O)C[C@@H]2N(C)CC3)cc1OC1CCCCC1 ; COc1ccc([C@@]23C=C[C@@H](O)C[C@@H]2N(C)CC3)cc1OC1CCCCC1 ; COc1ccc([C@@]23C=C[C@H](O)C[C@@H]2N(C)CC3)cc1OC1CCCCC1]

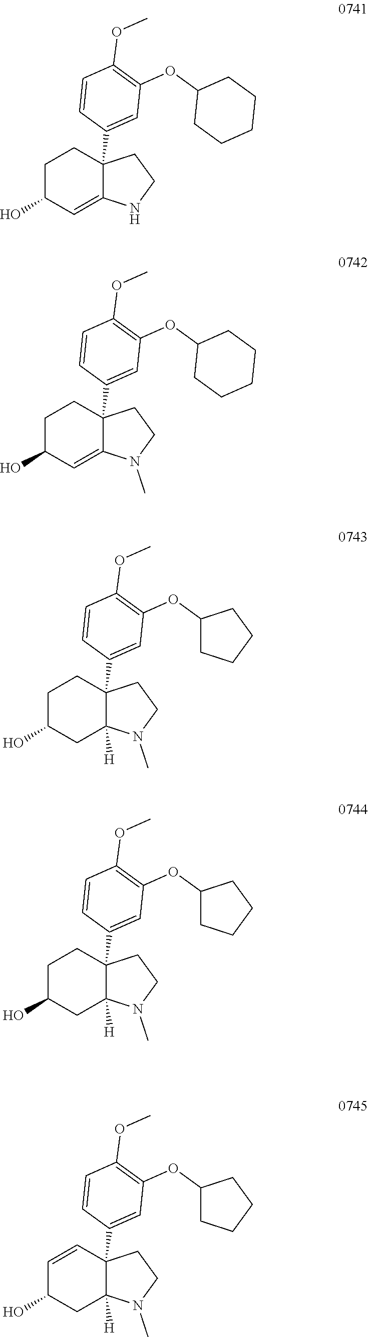 [SMILES: COc1ccc([C@]23CCNC2=C[C@H](O)CC3)cc1OC1CCCCC1 ; COc1ccc([C@@]23CC[C@H](O)C=C2N(C)CC3)cc1OC1CCCCC1 ; COc1ccc([C@@]23CC[C@@H](O)C[C@@H]2N(C)CC3)cc1OC1CCCC1 ; COc1ccc([C@@]23CC[C@H](O)C[C@@H]2N(C)CC3)cc1OC1CCCC1 ; COc1ccc([C@@]23C=C[C@@H](O)C[C@@H]2N(C)CC3)cc1OC1CCCC1]

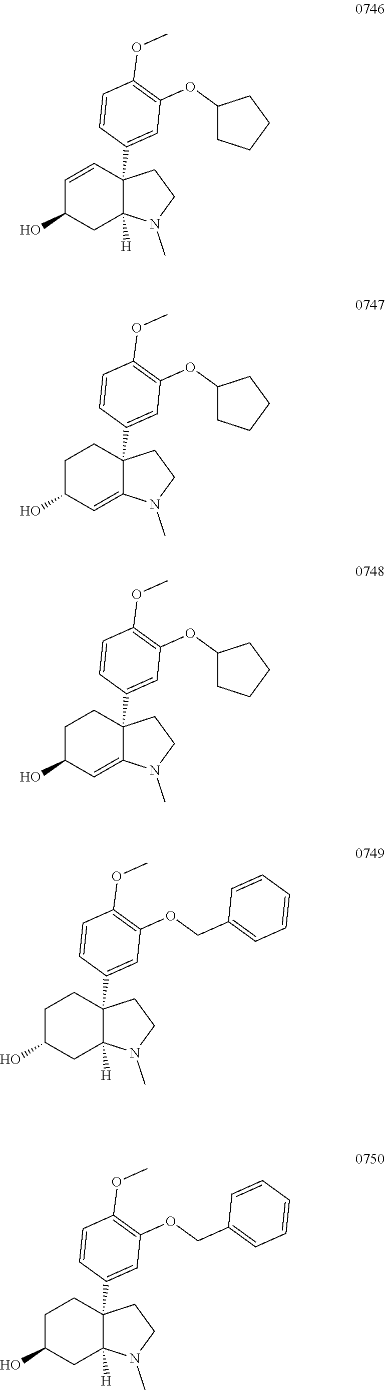

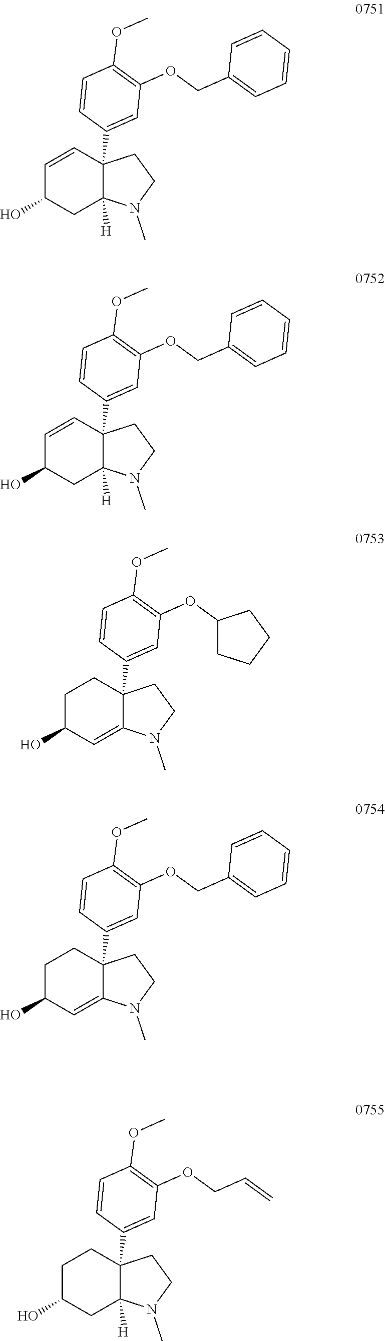

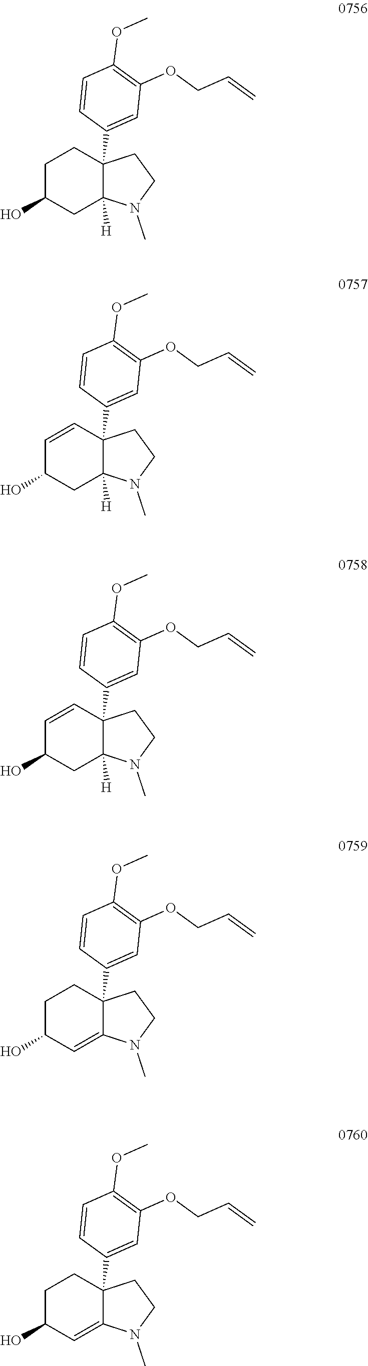 [SMILES: C=CCOc1cc([C@@]23CC[C@H](O)C[C@@H]2N(C)CC3)ccc1OC ; C=CCOc1cc([C@@]23C=C[C@@H](O)C[C@@H]2N(C)CC3)ccc1OC ; C=CCOc1cc([C@@]23C=C[C@H](O)C[C@@H]2N(C)CC3)ccc1OC ; C=CCOc1cc([C@@]23CC[C@@H](O)C=C2N(C)CC3)ccc1OC ; C=CCOc1cc([C@@]23CC[C@H](O)C=C2N(C)CC3)ccc1OC]

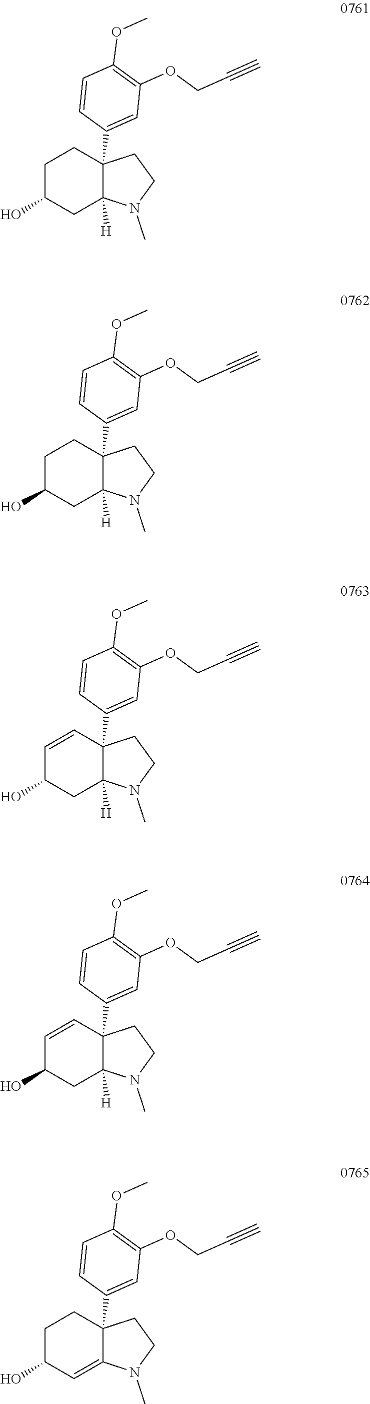 [SMILES: C#CCOc1cc([C@@]23CC[C@@H](O)C[C@@H]2N(C)CC3)ccc1OC ; C#CCOc1cc([C@@]23CC[C@H](O)C[C@@H]2N(C)CC3)ccc1OC ; C#CCOc1cc([C@@]23C=C[C@@H](O)C[C@@H]2N(C)CC3)ccc1OC ; C#CCOc1cc([C@@]23C=C[C@H](O)C[C@@H]2N(C)CC3)ccc1OC ; C#CCOc1cc([C@@]23CC[C@@H](O)C=C2N(C)CC3)ccc1OC]

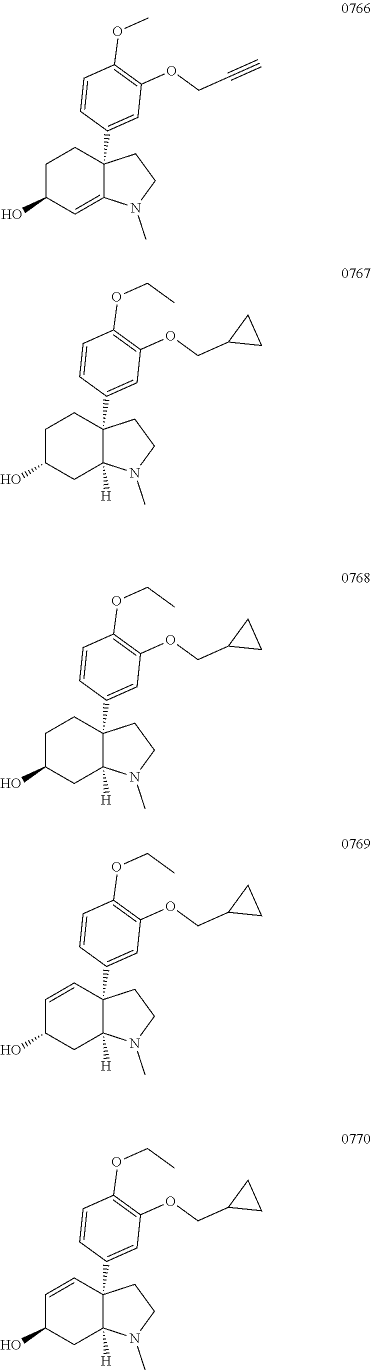

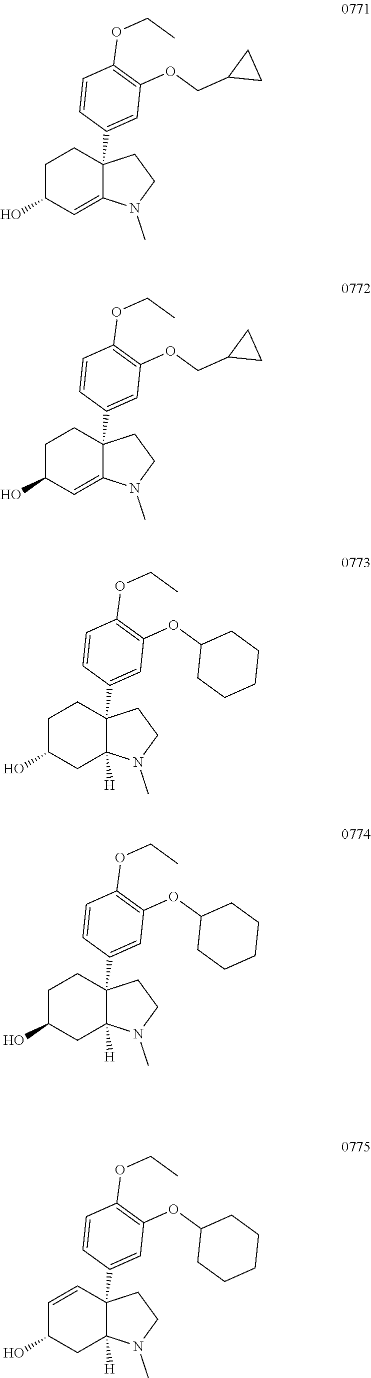 [SMILES: CCOc1ccc([C@@]23CC[C@@H](O)C=C2N(C)CC3)cc1OCC1CC1 ; CCOc1ccc([C@@]23CC[C@H](O)C=C2N(C)CC3)cc1OCC1CC1 ; CCOc1ccc([C@@]23CC[C@@H](O)C[C@@H]2N(C)CC3)cc1OC1CCCCC1 ; CCOc1ccc([C@@]23CC[C@H](O)C[C@@H]2N(C)CC3)cc1OC1CCCCC1 ; CCOc1ccc([C@@]23C=C[C@@H](O)C[C@@H]2N(C)CC3)cc1OC1CCCCC1]

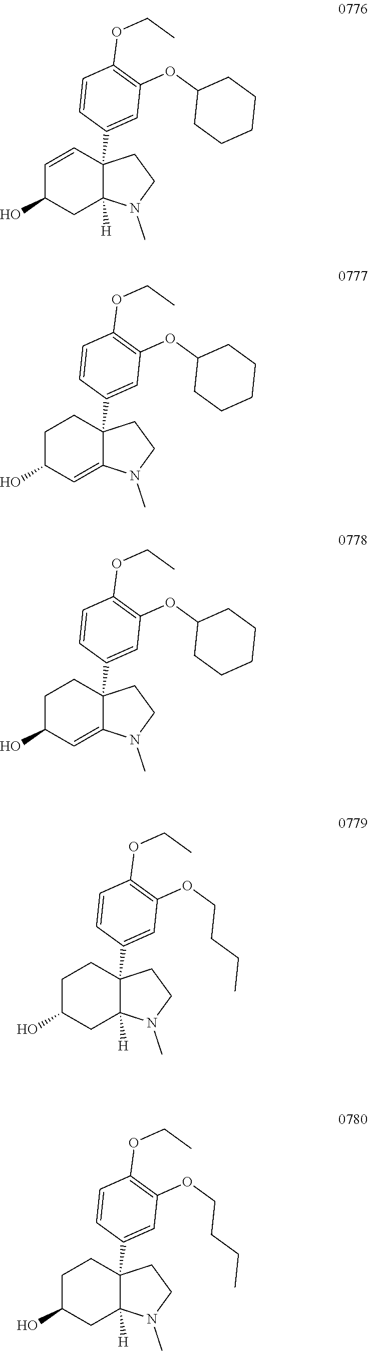

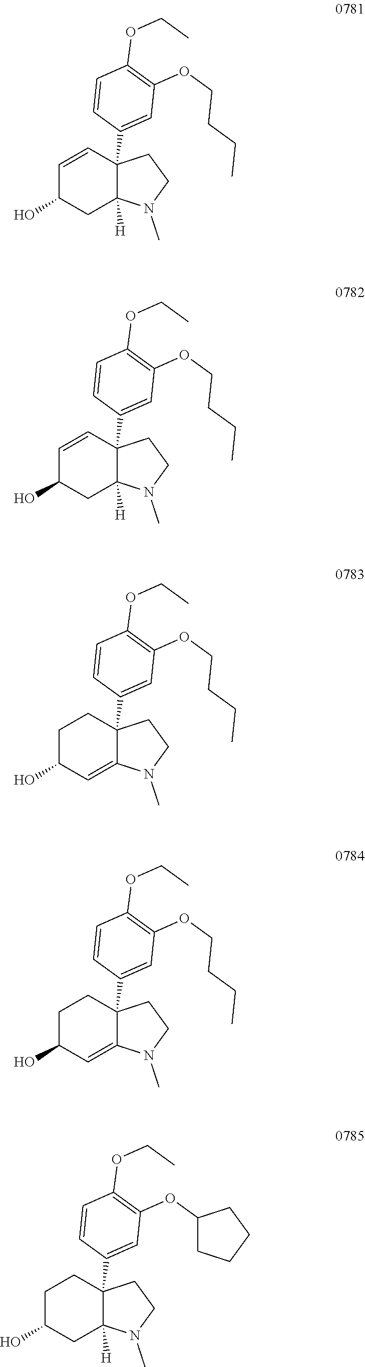 [SMILES: CCCCOc1cc([C@@]23C=C[C@@H](O)C[C@@H]2N(C)CC3)ccc1OCC ; CCCCOc1cc([C@@]23C=C[C@H](O)C[C@@H]2N(C)CC3)ccc1OCC ; CCCCOc1cc([C@@]23CC[C@@H](O)C=C2N(C)CC3)ccc1OCC ; CCCCOc1cc([C@@]23CC[C@H](O)C=C2N(C)CC3)ccc1OCC ; CCOc1ccc([C@@]23CC[C@@H](O)C[C@@H]2N(C)CC3)cc1OC1CCCC1]

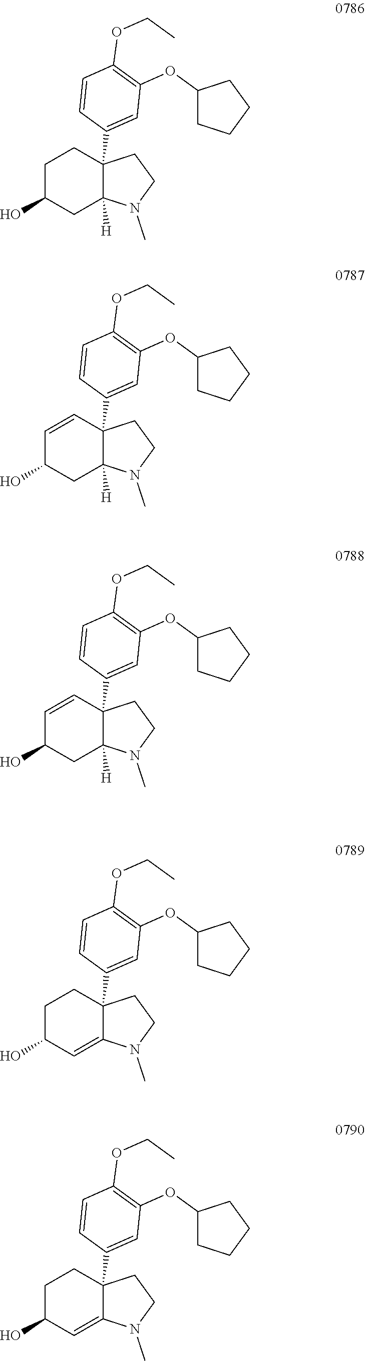

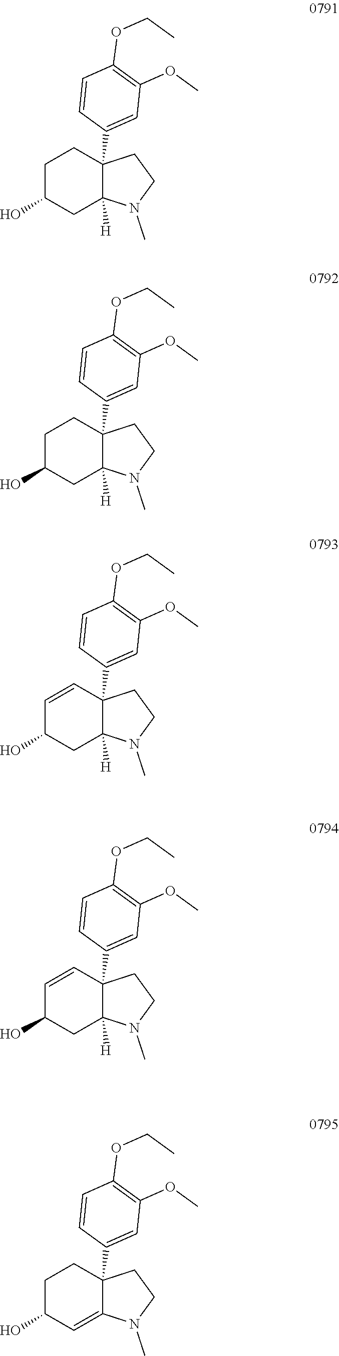

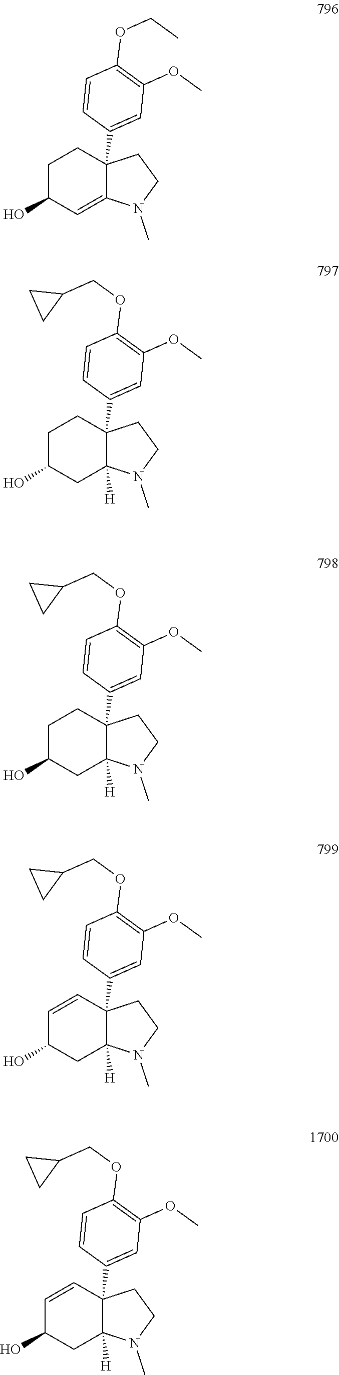 [SMILES: CCOc1ccc([C@@]23CC[C@H](O)C=C2N(C)CC3)cc1OC ; COc1cc([C@@]23CC[C@@H](O)C[C@@H]2N(C)CC3)ccc1OCC1CC1 ; COc1cc([C@@]23CC[C@H](O)C[C@@H]2N(C)CC3)ccc1OCC1CC1 ; COc1cc([C@@]23C=C[C@@H](O)C[C@@H]2N(C)CC3)ccc1OCC1CC1 ; COc1cc([C@@]23C=C[C@H](O)C[C@@H]2N(C)CC3)ccc1OCC1CC1]

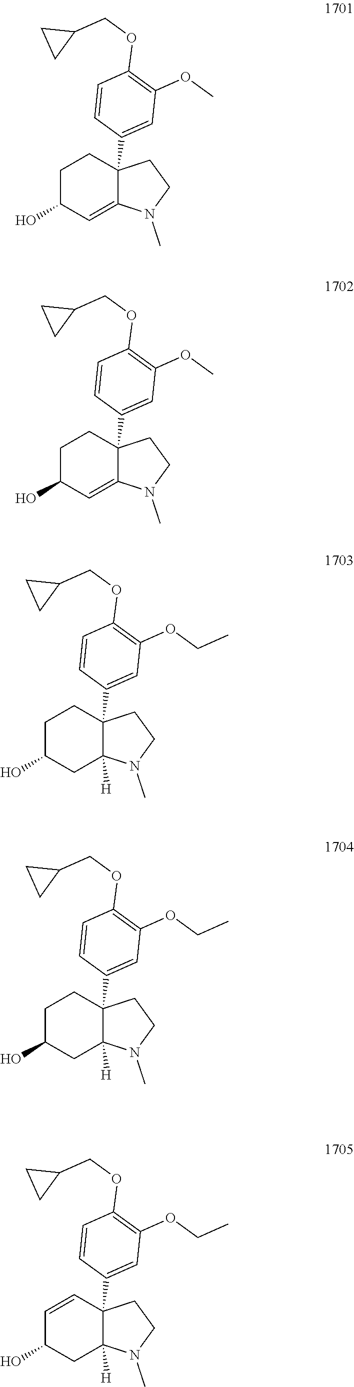 [SMILES: COc1cc([C@@]23CC[C@@H](O)C=C2N(C)CC3)ccc1OCC1CC1 ; COc1cc([C@@]23CC[C@H](O)C=C2N(C)CC3)ccc1OCC1CC1 ; CCOc1cc([C@@]23CC[C@@H](O)C[C@@H]2N(C)CC3)ccc1OCC1CC1 ; CCOc1cc([C@@]23CC[C@H](O)C[C@@H]2N(C)CC3)ccc1OCC1CC1 ; CCOc1cc([C@@]23C=C[C@@H](O)C[C@@H]2N(C)CC3)ccc1OCC1CC1]

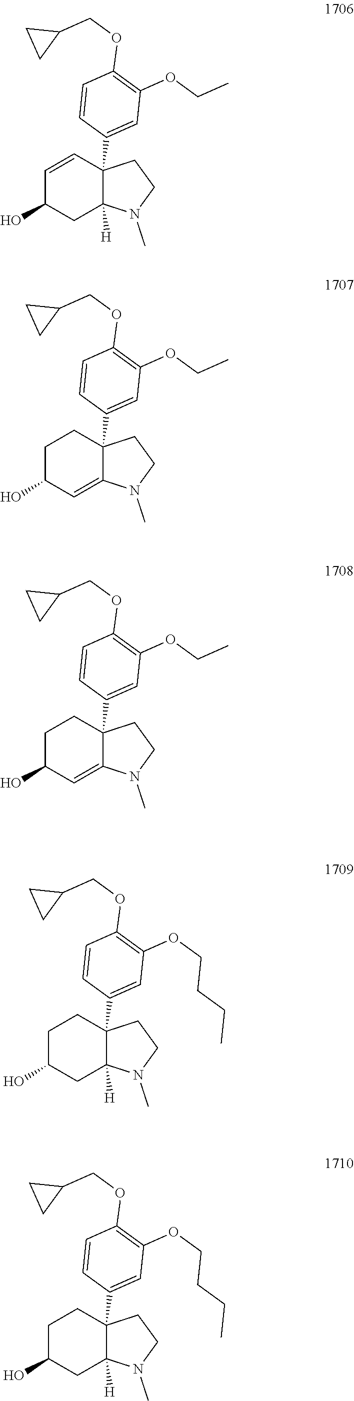 [SMILES: CCOc1cc([C@@]23C=C[C@H](O)C[C@@H]2N(C)CC3)ccc1OCC1CC1 ; CCOc1cc([C@@]23CC[C@@H](O)C=C2N(C)CC3)ccc1OCC1CC1 ; CCOc1cc([C@@]23CC[C@H](O)C=C2N(C)CC3)ccc1OCC1CC1 ; CCCCOc1cc([C@@]23CC[C@@H](O)C[C@@H]2N(C)CC3)ccc1OCC1CC1 ; CCCCOc1cc([C@@]23CC[C@H](O)C[C@@H]2N(C)CC3)ccc1OCC1CC1]

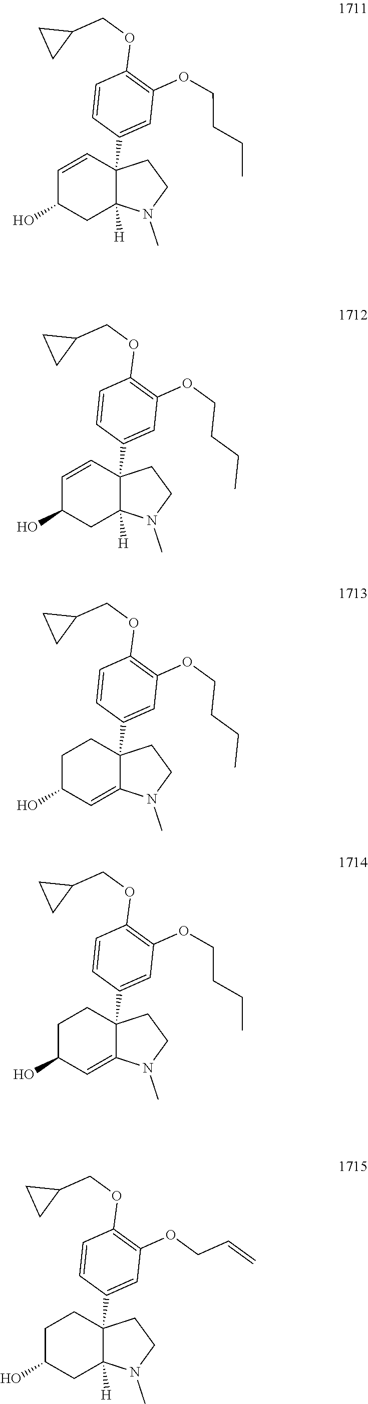 [SMILES: CCCCOc1cc([C@@]23C=C[C@@H](O)C[C@@H]2N(C)CC3)ccc1OCC1CC1 ; CCCCOc1cc([C@@]23C=C[C@H](O)C[C@@H]2N(C)CC3)ccc1OCC1CC1 ; CCCCOc1cc([C@@]23CC[C@@H](O)C=C2N(C)CC3)ccc1OCC1CC1 ; CCCCOc1cc([C@@]23CC[C@H](O)C=C2N(C)CC3)ccc1OCC1CC1 ; C=CCOc1cc([C@@]23CC[C@@H](O)C[C@@H]2N(C)CC3)ccc1OCC1CC1]

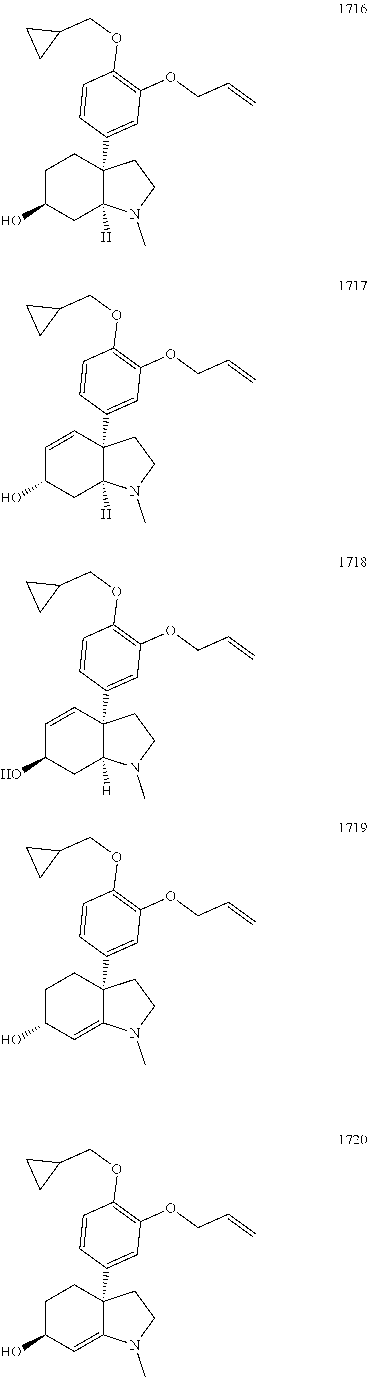 [SMILES: C=CCOc1cc([C@@]23CC[C@H](O)C[C@@H]2N(C)CC3)ccc1OCC1CC1 ; C=CCOc1cc([C@@]23C=C[C@@H](O)C[C@@H]2N(C)CC3)ccc1OCC1CC1 ; C=CCOc1cc([C@@]23C=C[C@H](O)C[C@@H]2N(C)CC3)ccc1OCC1CC1 ; C=CCOc1cc([C@@]23CC[C@@H](O)C=C2N(C)CC3)ccc1OCC1CC1 ; C=CCOc1cc([C@@]23CC[C@H](O)C=C2N(C)CC3)ccc1OCC1CC1]

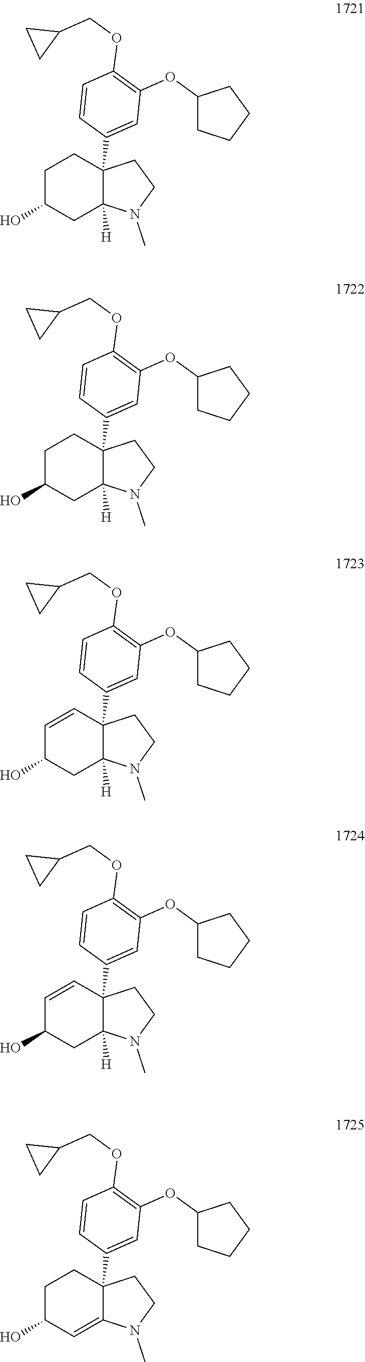 [SMILES: CN1CC[C@]2(c3ccc(OCC4CC4)c(OC4CCCC4)c3)CC[C@@H](O)C[C@H]12 ; CN1CC[C@]2(c3ccc(OCC4CC4)c(OC4CCCC4)c3)CC[C@H](O)C[C@H]12 ; CN1CC[C@]2(c3ccc(OCC4CC4)c(OC4CCCC4)c3)C=C[C@@H](O)C[C@H]12 ; CN1CC[C@]2(c3ccc(OCC4CC4)c(OC4CCCC4)c3)C=C[C@H](O)C[C@H]12 ; CN1CC[C@]2(c3ccc(OCC4CC4)c(OC4CCCC4)c3)CC[C@@H](O)C=C12]

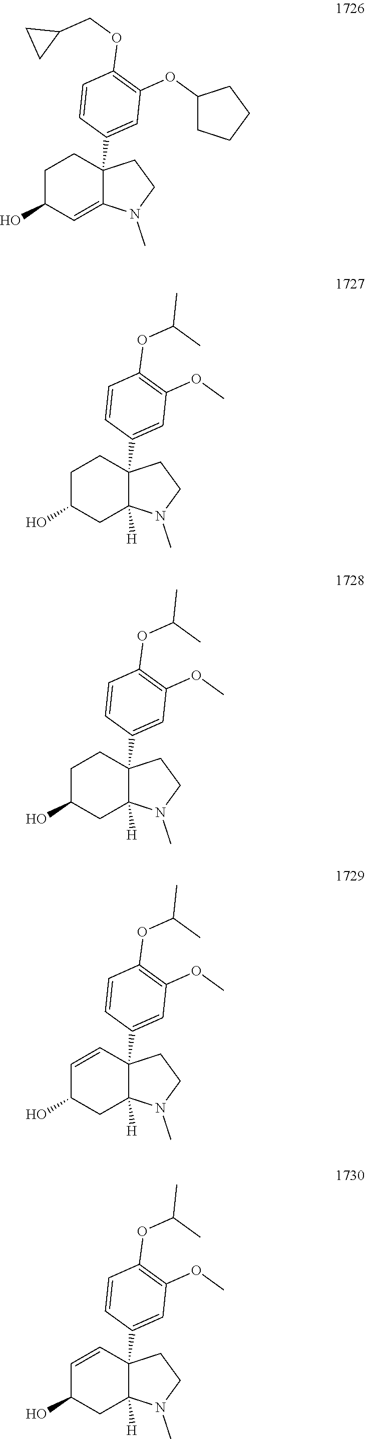

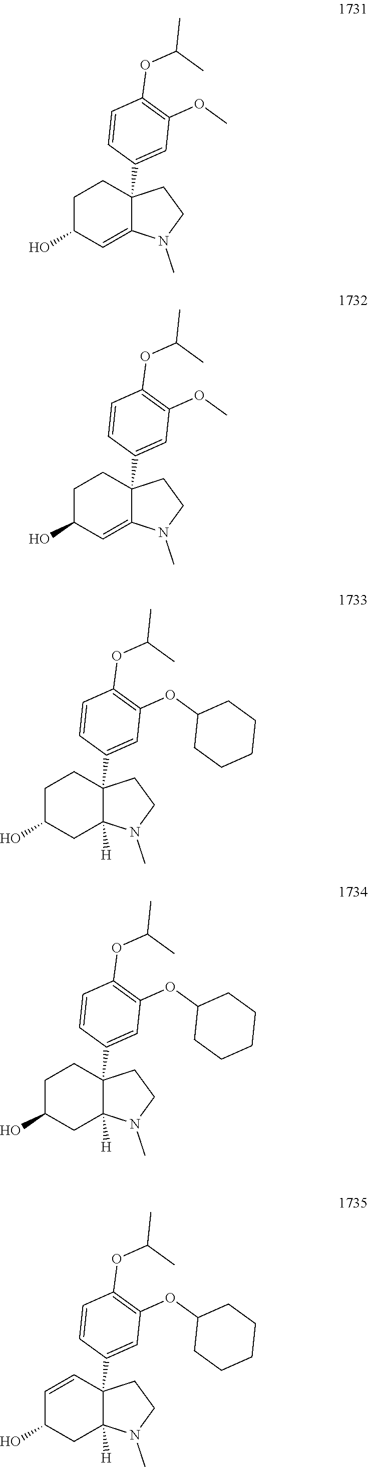

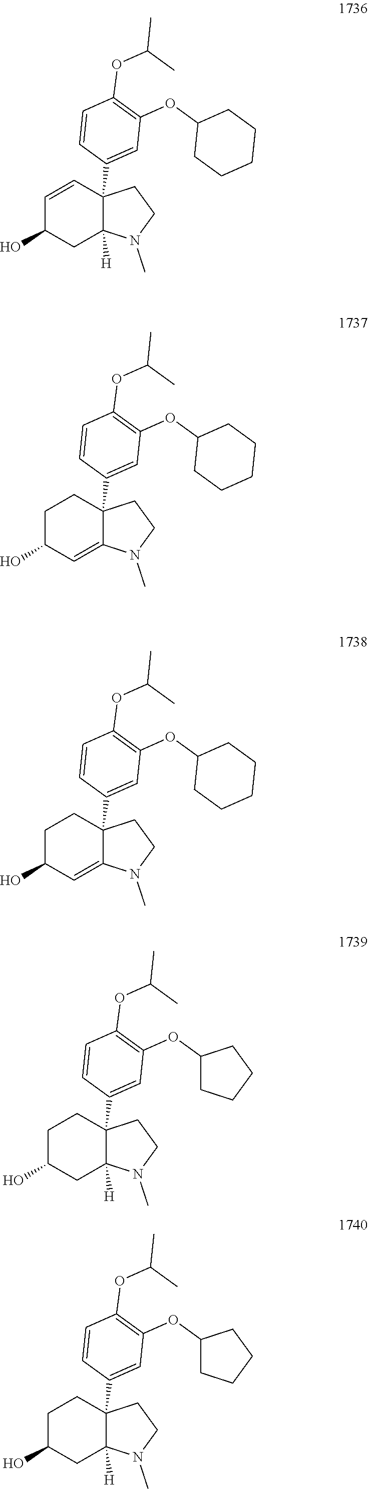

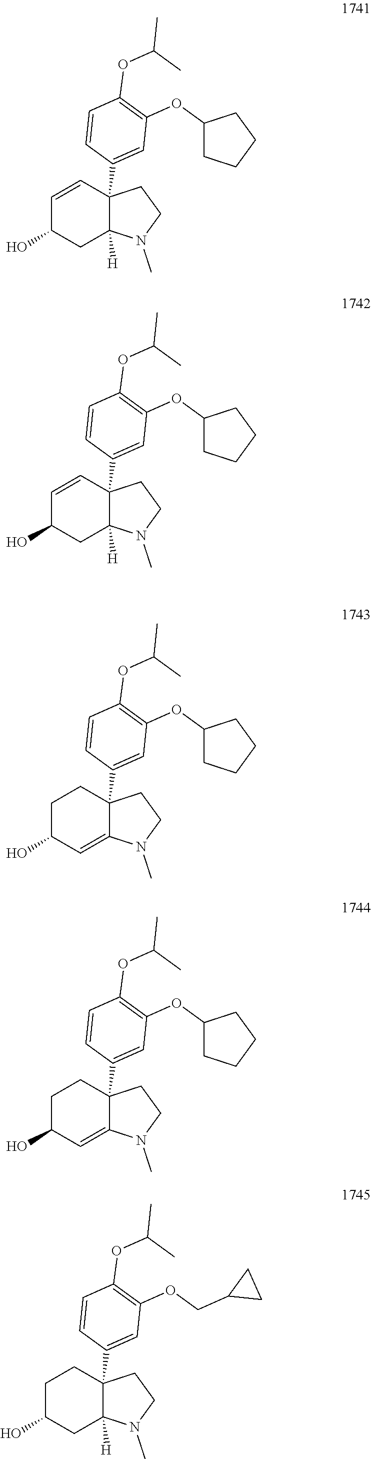

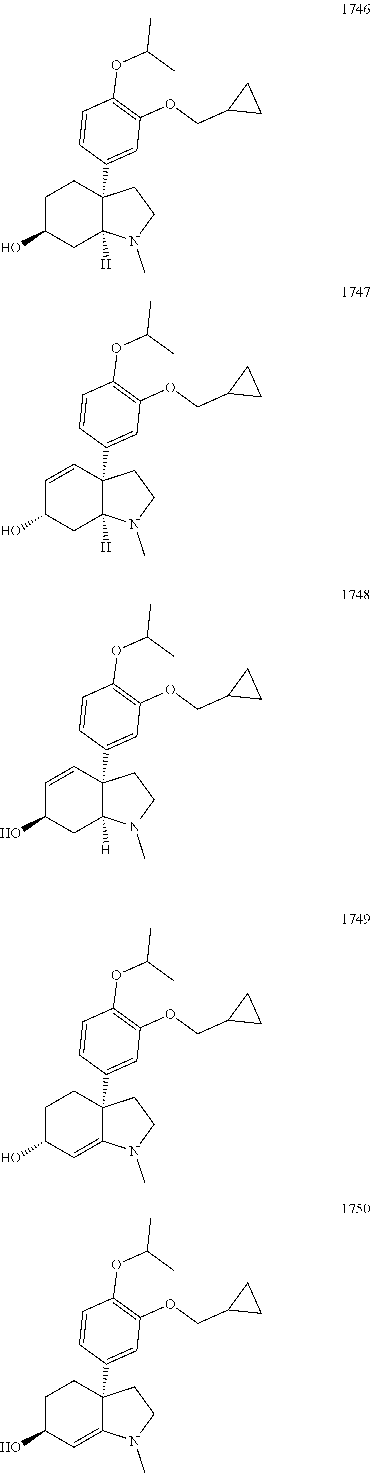

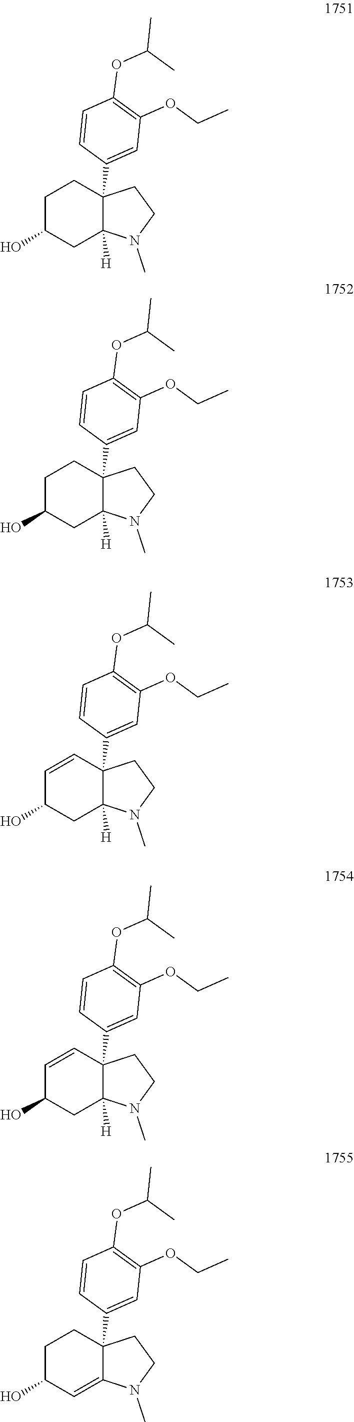

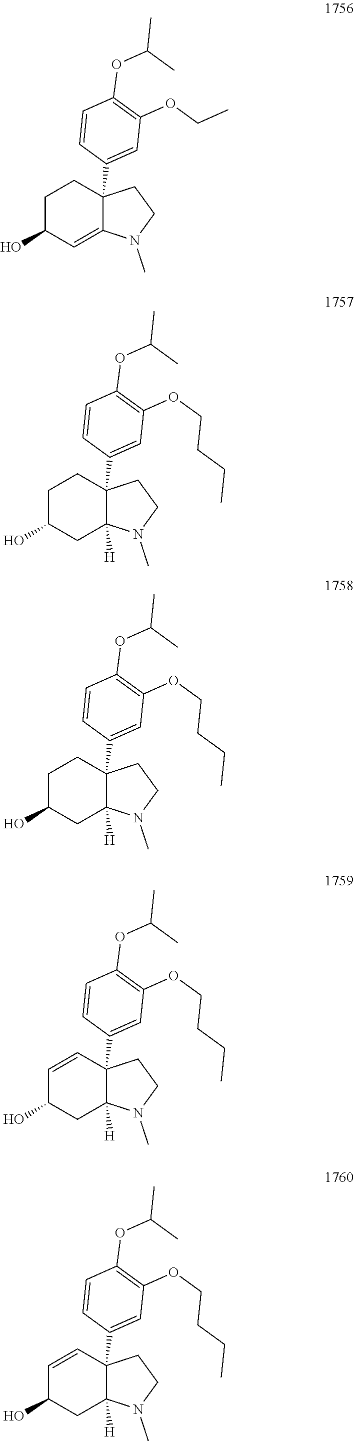

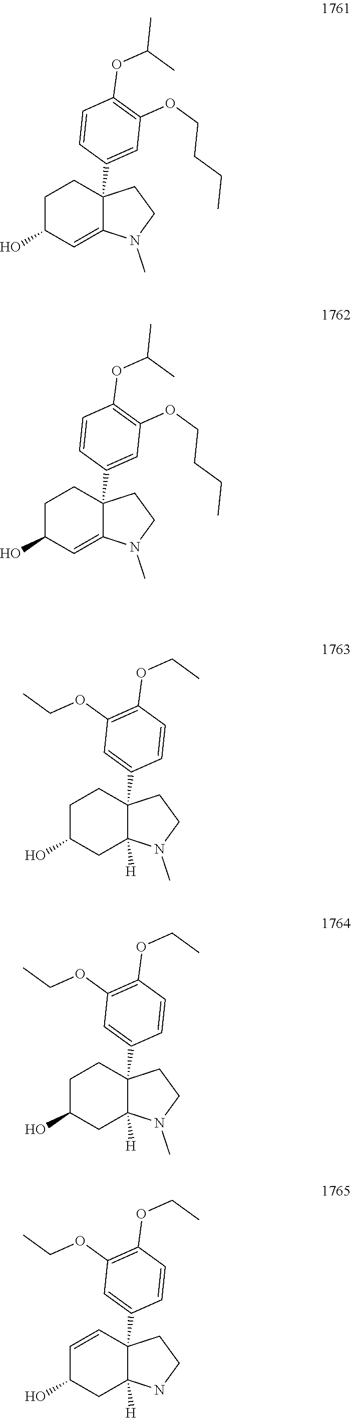

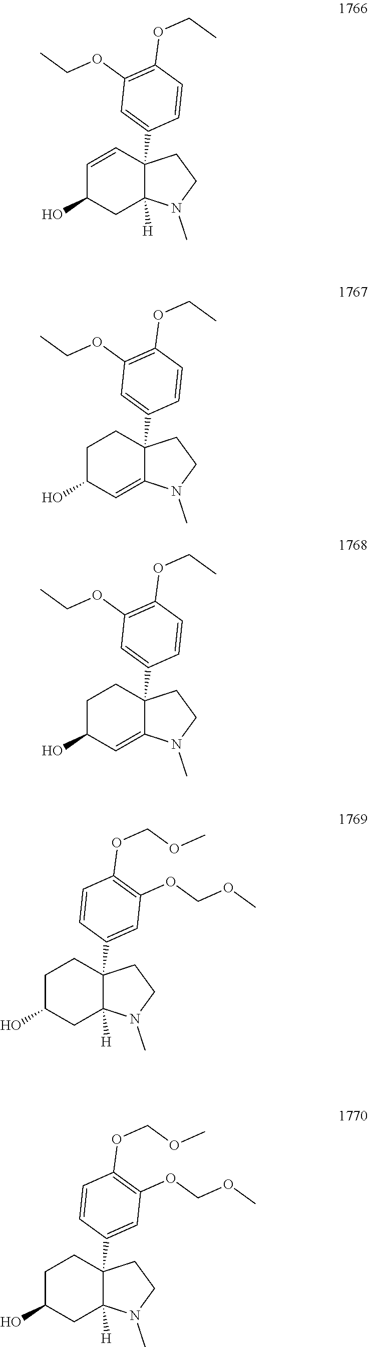

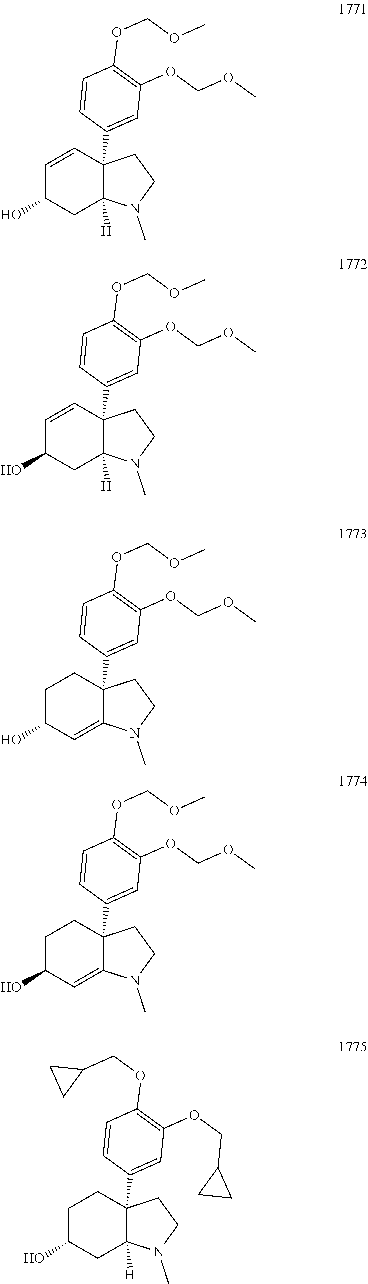

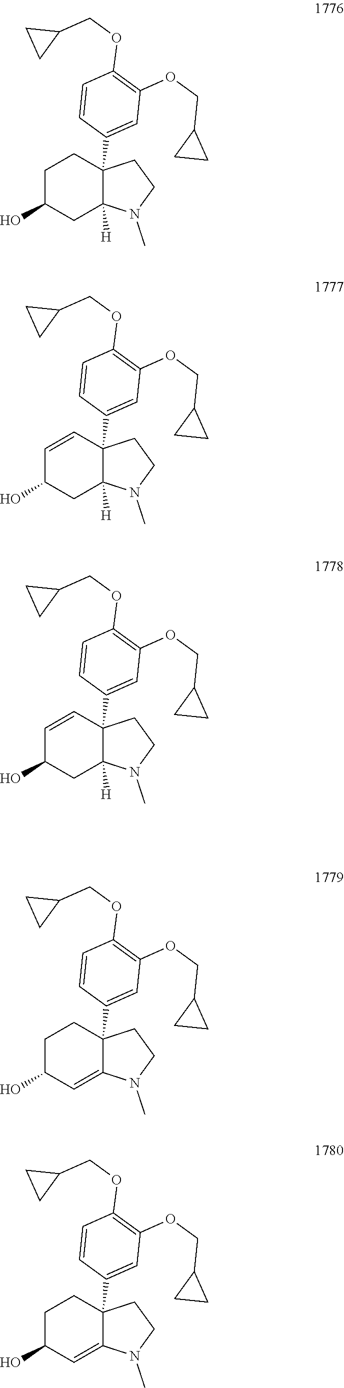 [SMILES: CN1CC[C@]2(c3ccc(OCC4CC4)c(OCC4CC4)c3)CC[C@H](O)C[C@H]12 ; CN1CC[C@]2(c3ccc(OCC4CC4)c(OCC4CC4)c3)C=C[C@@H](O)C[C@H]12 ; CN1CC[C@]2(c3ccc(OCC4CC4)c(OCC4CC4)c3)C=C[C@H](O)C[C@H]12 ; CN1CC[C@]2(c3ccc(OCC4CC4)c(OCC4CC4)c3)CC[C@@H](O)C=C12 ; CN1CC[C@]2(c3ccc(OCC4CC4)c(OCC4CC4)c3)CC[C@H](O)C=C12]

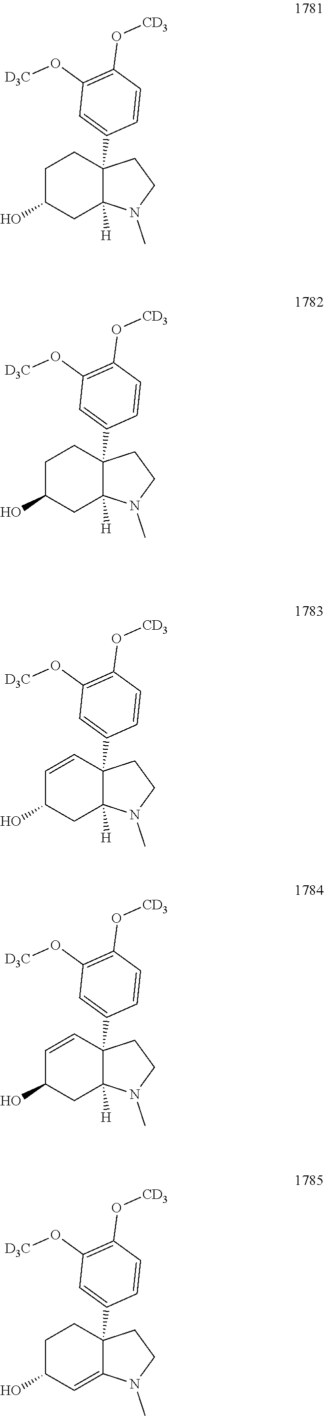 [SMILES: [2H]C([2H])([2H])Oc1ccc([C@@]23CC[C@@H](O)C[C@@H]2N(C)CC3)cc1OC([2H])([2H])[2H] ; [2H]C([2H])([2H])Oc1ccc([C@@]23CC[C@H](O)C[C@@H]2N(C)CC3)cc1OC([2H])([2H])[2H] ; [2H]C([2H])([2H])Oc1ccc([C@@]23C=C[C@@H](O)C[C@@H]2N(C)CC3)cc1OC([2H])([2H])[2H] ; [2H]C([2H])([2H])Oc1ccc([C@@]23C=C[C@H](O)C[C@@H]2N(C)CC3)cc1OC([2H])([2H])[2H] ; [2H]C([2H])([2H])Oc1ccc([C@@]23CC[C@@H](O)C=C2N(C)CC3)cc1OC([2H])([2H])[2H]]

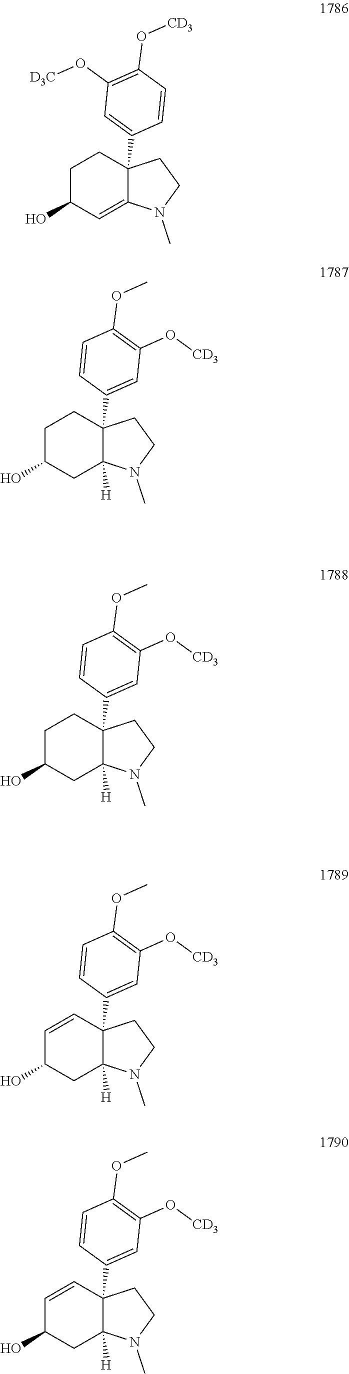

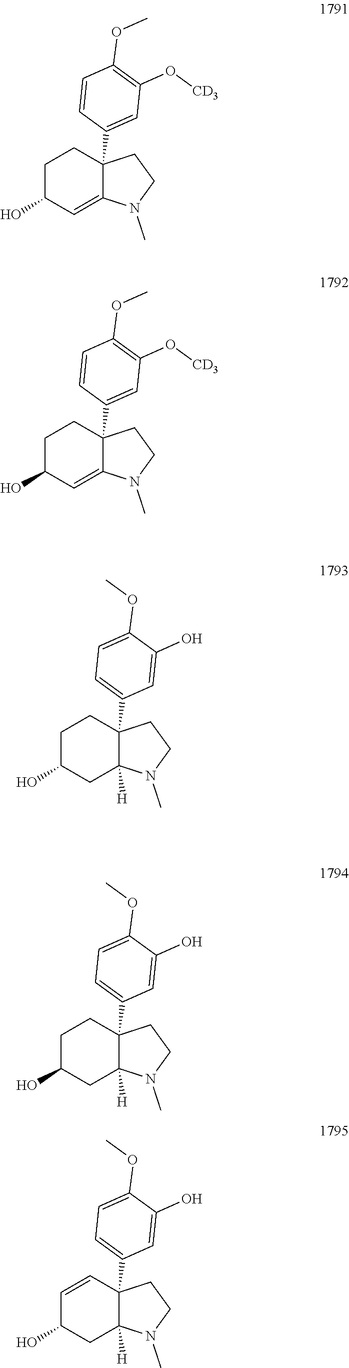

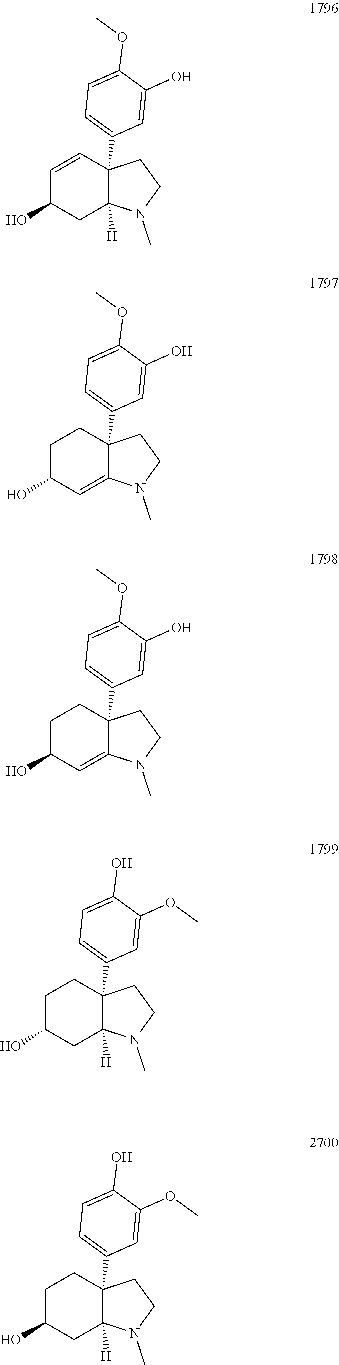 [SMILES: COc1ccc([C@@]23C=C[C@H](O)C[C@@H]2N(C)CC3)cc1O ; COc1ccc([C@@]23CC[C@@H](O)C=C2N(C)CC3)cc1O ; COc1ccc([C@@]23CC[C@H](O)C=C2N(C)CC3)cc1O ; COc1cc([C@@]23CC[C@@H](O)C[C@@H]2N(C)CC3)ccc1O ; COc1cc([C@@]23CC[C@H](O)C[C@@H]2N(C)CC3)ccc1O]

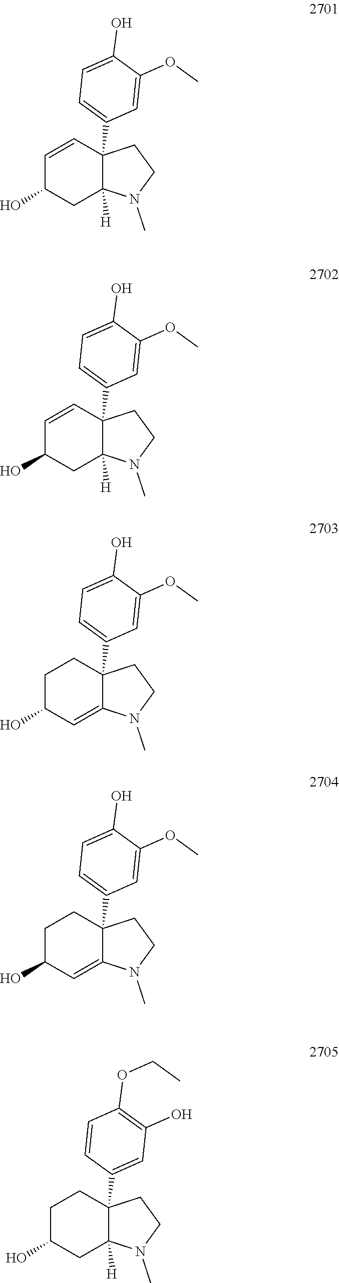 [SMILES: COc1cc([C@@]23C=C[C@@H](O)C[C@@H]2N(C)CC3)ccc1O ; COc1cc([C@@]23C=C[C@H](O)C[C@@H]2N(C)CC3)ccc1O ; COc1cc([C@@]23CC[C@@H](O)C=C2N(C)CC3)ccc1O ; COc1cc([C@@]23CC[C@H](O)C=C2N(C)CC3)ccc1O ; CCOc1ccc([C@@]23CC[C@@H](O)C[C@@H]2N(C)CC3)cc1O]

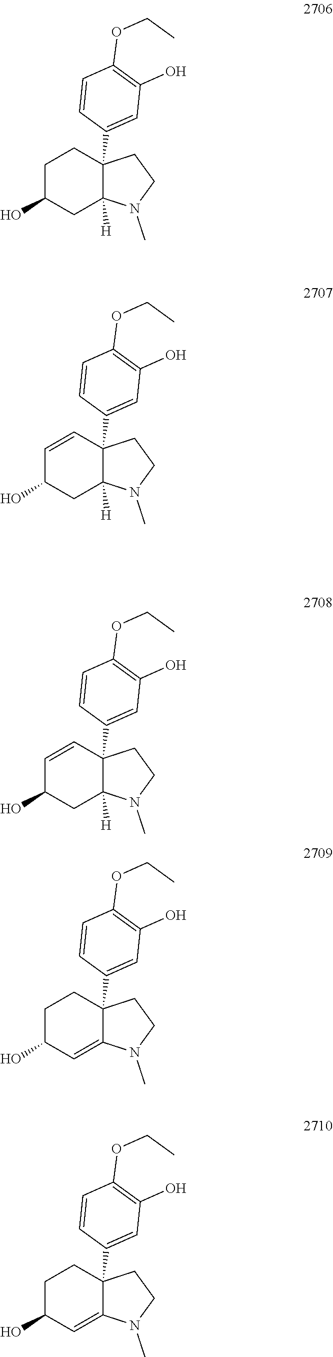

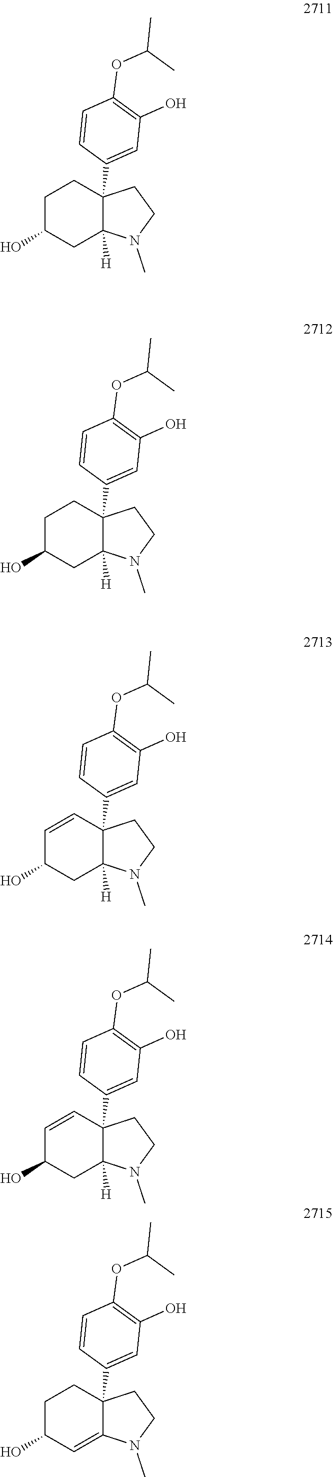

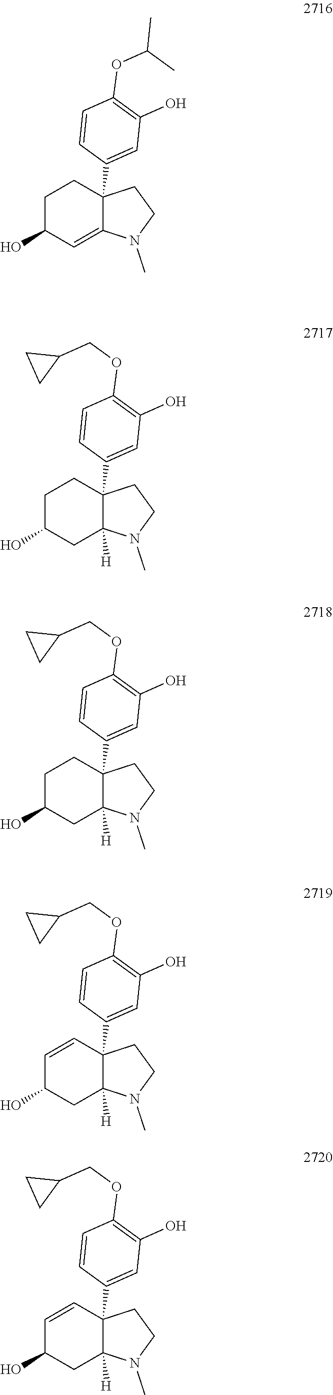 [SMILES: CC(C)Oc1ccc([C@@]23CC[C@H](O)C=C2N(C)CC3)cc1O ; CN1CC[C@]2(c3ccc(OCC4CC4)c(O)c3)CC[C@@H](O)C[C@H]12 ; CN1CC[C@]2(c3ccc(OCC4CC4)c(O)c3)CC[C@H](O)C[C@H]12 ; CN1CC[C@]2(c3ccc(OCC4CC4)c(O)c3)C=C[C@@H](O)C[C@H]12 ; CN1CC[C@]2(c3ccc(OCC4CC4)c(O)c3)C=C[C@H](O)C[C@H]12]

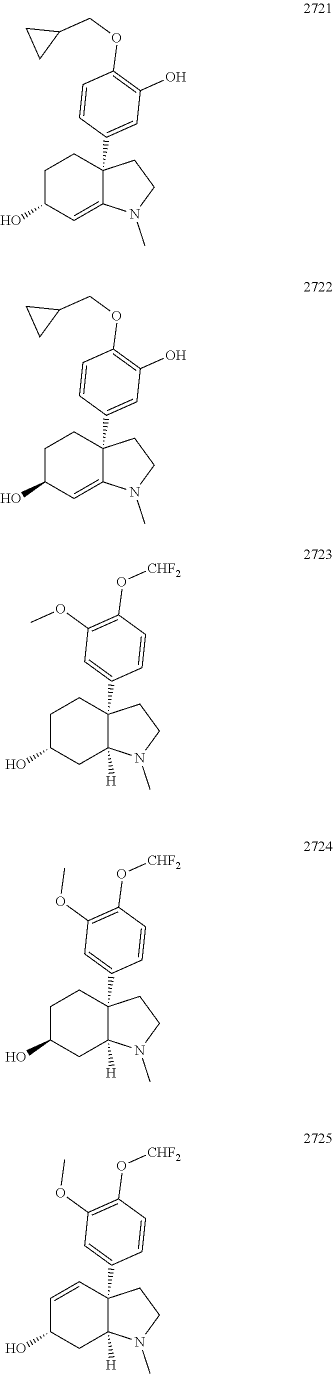

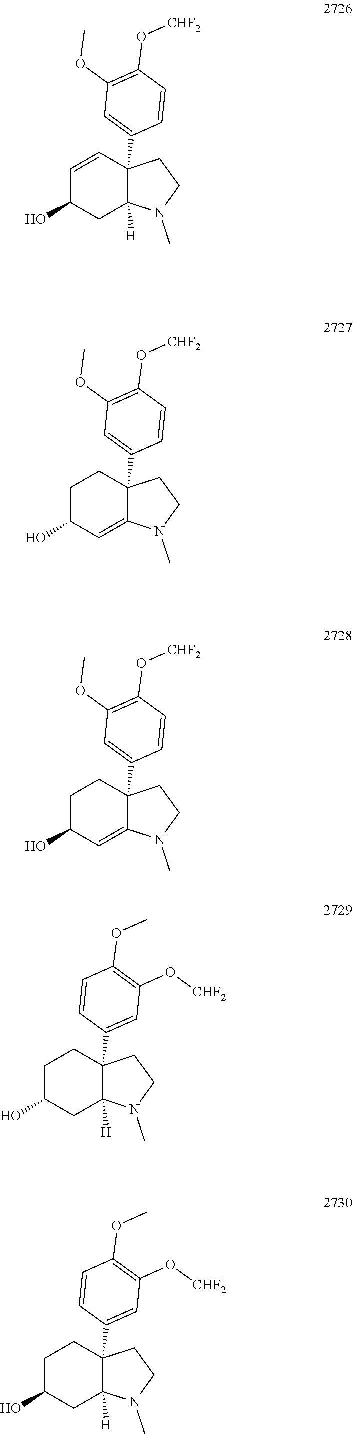 [SMILES: COc1cc([C@@]23C=C[C@H](O)C[C@@H]2N(C)CC3)ccc1OC(F)F ; COc1cc([C@@]23CC[C@@H](O)C=C2N(C)CC3)ccc1OC(F)F ; COc1cc([C@@]23CC[C@H](O)C=C2N(C)CC3)ccc1OC(F)F ; COc1ccc([C@@]23CC[C@@H](O)C[C@@H]2N(C)CC3)cc1OC(F)F ; COc1ccc([C@@]23CC[C@H](O)C[C@@H]2N(C)CC3)cc1OC(F)F]

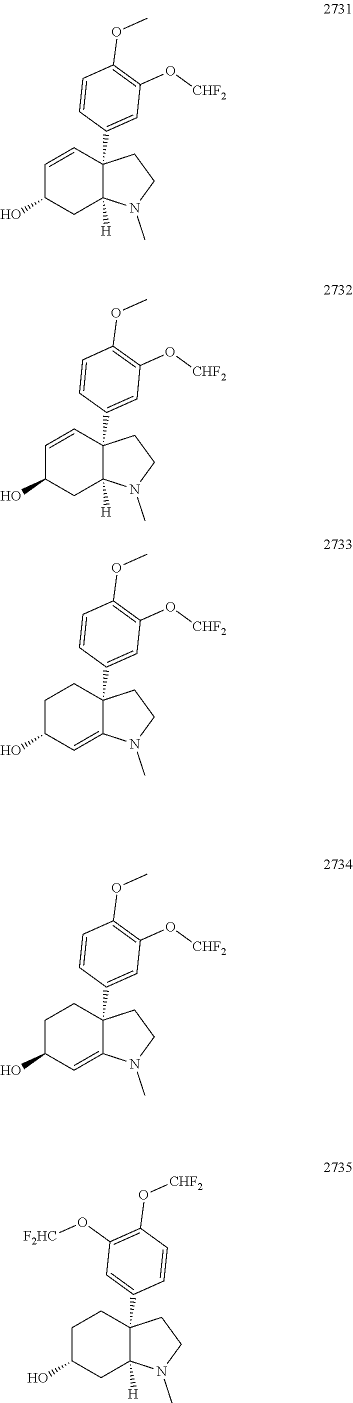

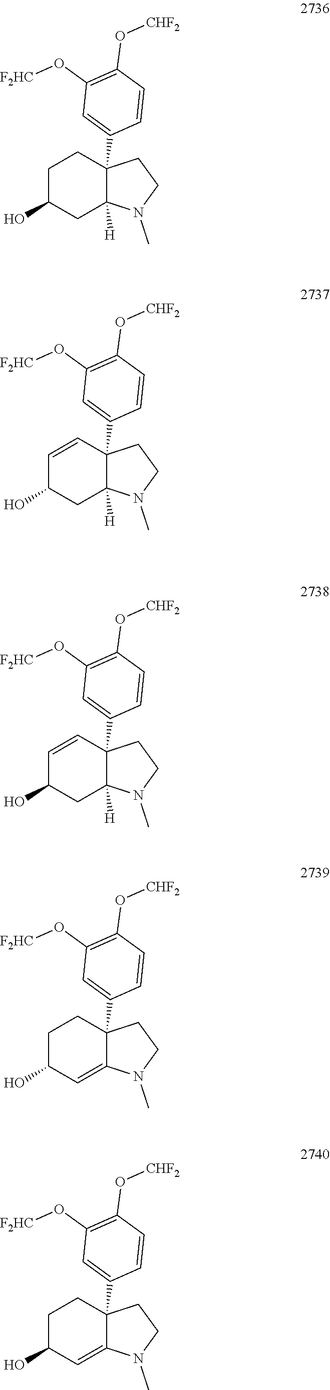 [SMILES: CN1CC[C@]2(c3ccc(OC(F)F)c(OC(F)F)c3)CC[C@H](O)C[C@H]12 ; CN1CC[C@]2(c3ccc(OC(F)F)c(OC(F)F)c3)C=C[C@@H](O)C[C@H]12 ; CN1CC[C@]2(c3ccc(OC(F)F)c(OC(F)F)c3)C=C[C@H](O)C[C@H]12 ; CN1CC[C@]2(c3ccc(OC(F)F)c(OC(F)F)c3)CC[C@@H](O)C=C12 ; CN1CC[C@]2(c3ccc(OC(F)F)c(OC(F)F)c3)CC[C@H](O)C=C12]

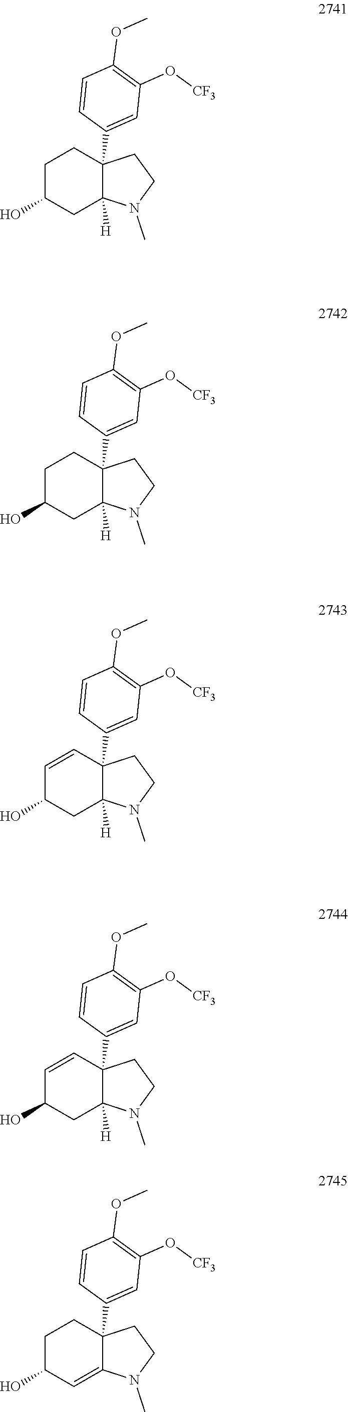

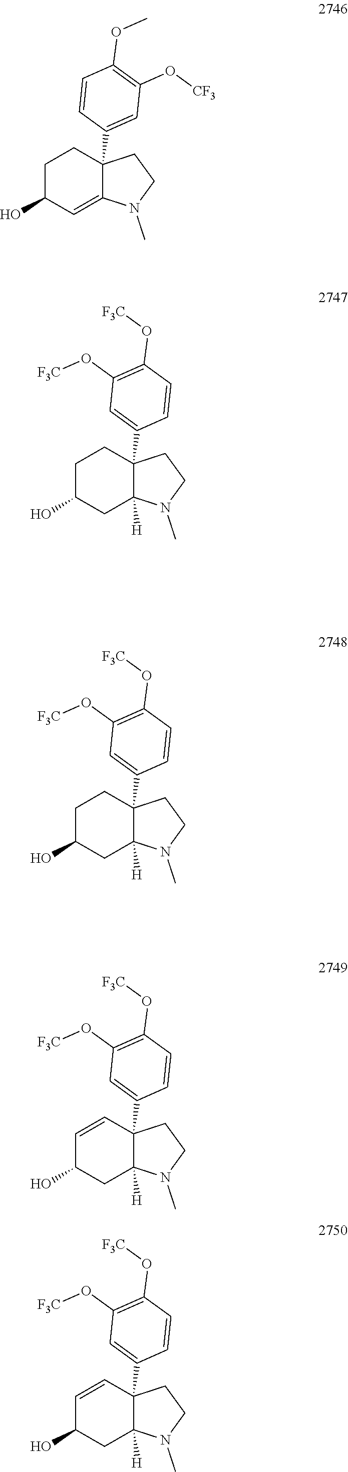 [SMILES: COc1ccc([C@@]23CC[C@H](O)C=C2N(C)CC3)cc1OC(F)(F)F ; CN1CC[C@]2(c3ccc(OC(F)(F)F)c(OC(F)(F)F)c3)CC[C@@H](O)C[C@H]12 ; CN1CC[C@]2(c3ccc(OC(F)(F)F)c(OC(F)(F)F)c3)CC[C@H](O)C[C@H]12 ; CN1CC[C@]2(c3ccc(OC(F)(F)F)c(OC(F)(F)F)c3)C=C[C@@H](O)C[C@H]12 ; CN1CC[C@]2(c3ccc(OC(F)(F)F)c(OC(F)(F)F)c3)C=C[C@H](O)C[C@H]12]

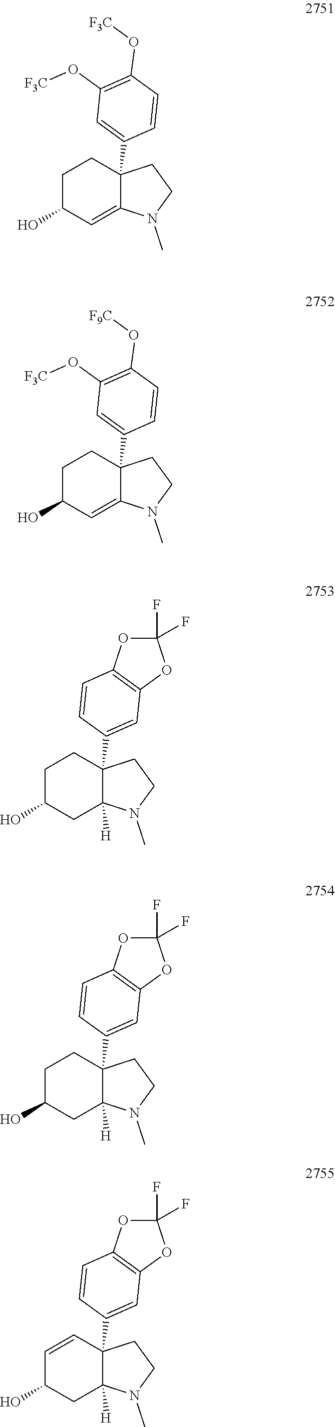

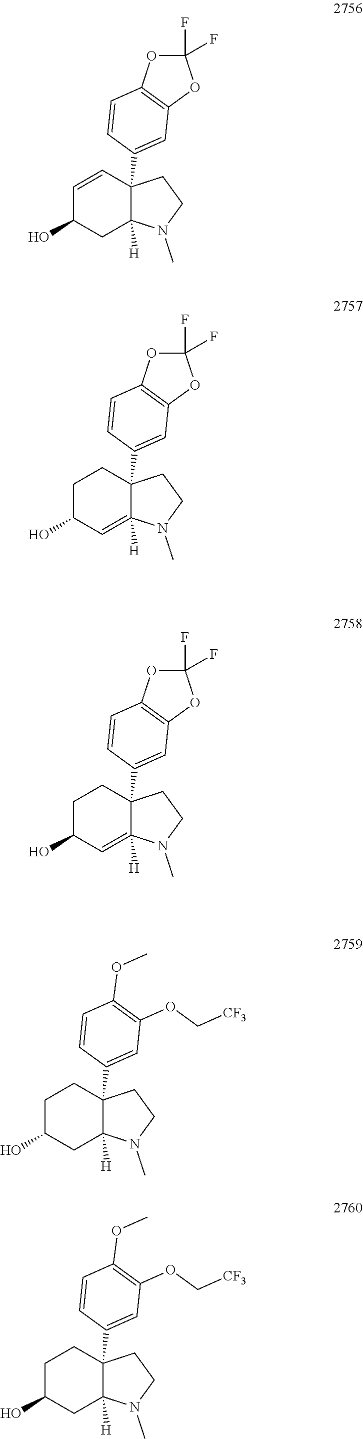

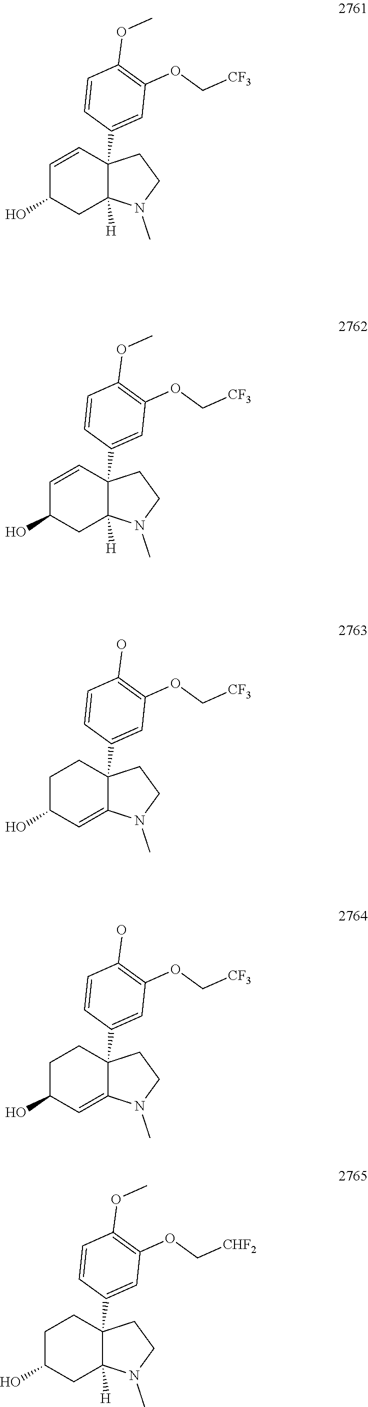

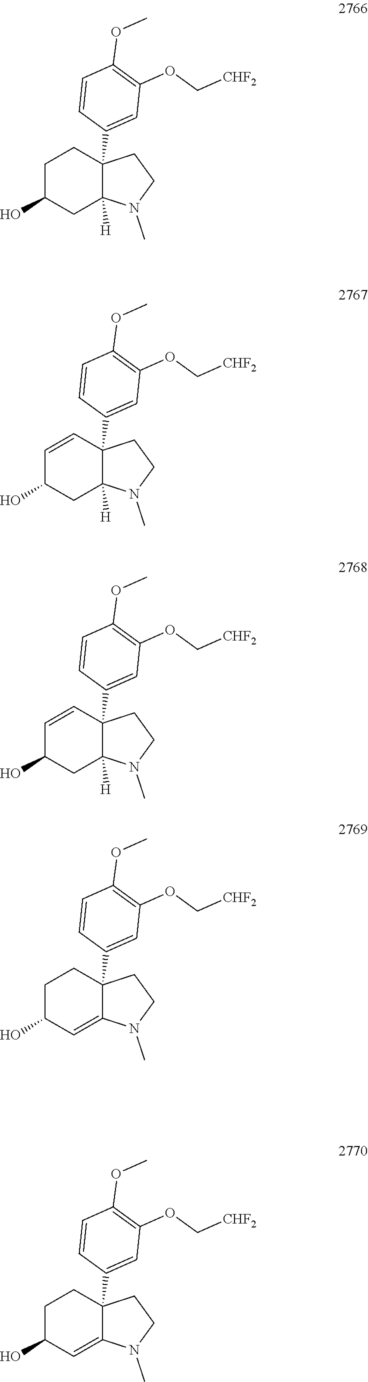

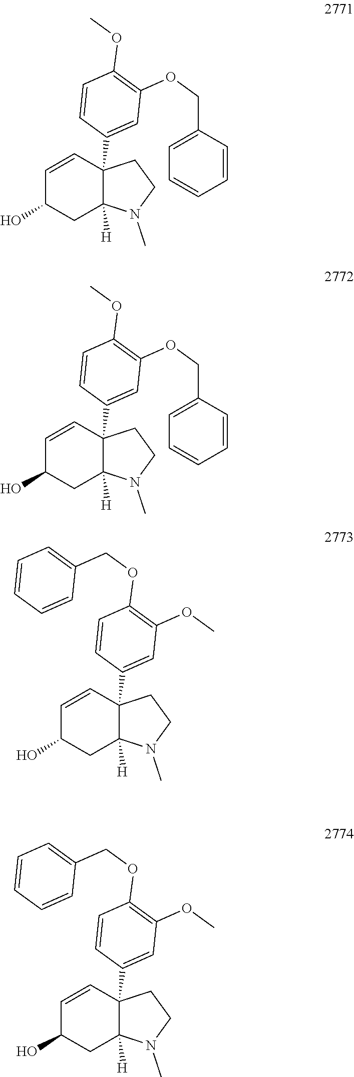 [SMILES: COc1ccc([C@@]23C=C[C@@H](O)C[C@@H]2N(C)CC3)cc1OCc1ccccc1 ; COc1ccc([C@@]23C=C[C@H](O)C[C@@H]2N(C)CC3)cc1OCc1ccccc1 ; COc1cc([C@@]23C=C[C@@H](O)C[C@@H]2N(C)CC3)ccc1OCc1ccccc1 ; COc1cc([C@@]23C=C[C@H](O)C[C@@H]2N(C)CC3)ccc1OCc1ccccc1]

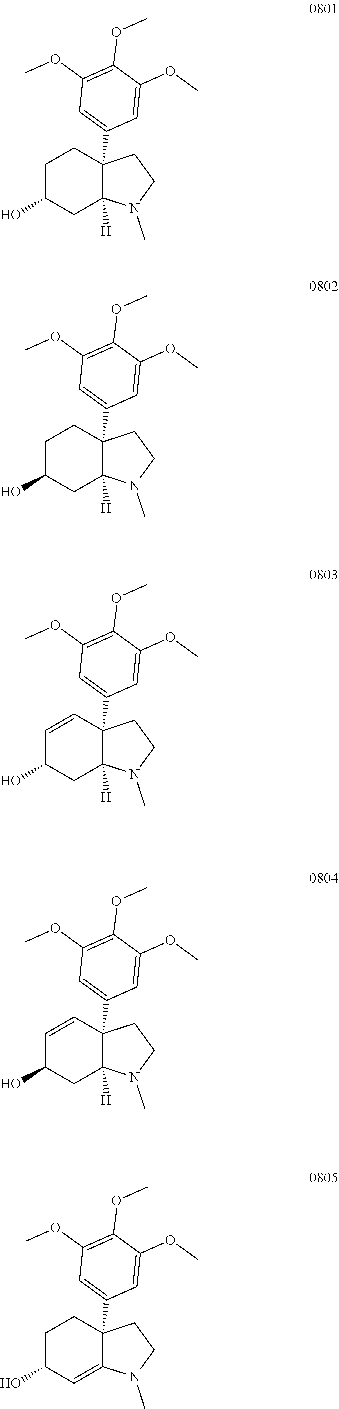 [SMILES: COc1cc([C@@]23CC[C@@H](O)C[C@@H]2N(C)CC3)cc(OC)c1OC ; COc1cc([C@@]23CC[C@H](O)C[C@@H]2N(C)CC3)cc(OC)c1OC ; COc1cc([C@@]23C=C[C@@H](O)C[C@@H]2N(C)CC3)cc(OC)c1OC ; COc1cc([C@@]23C=C[C@H](O)C[C@@H]2N(C)CC3)cc(OC)c1OC ; COc1cc([C@@]23CC[C@@H](O)C=C2N(C)CC3)cc(OC)c1OC]

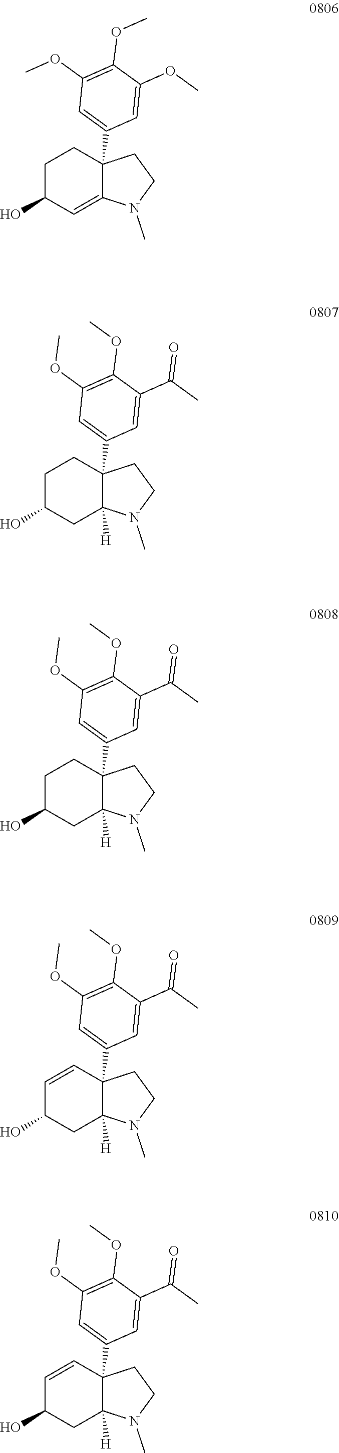 [SMILES: COc1cc([C@@]23CC[C@H](O)C=C2N(C)CC3)cc(OC)c1OC ; COc1cc([C@@]23CC[C@@H](O)C[C@@H]2N(C)CC3)cc(C(C)=O)c1OC ; COc1cc([C@@]23CC[C@H](O)C[C@@H]2N(C)CC3)cc(C(C)=O)c1OC ; COc1cc([C@@]23C=C[C@@H](O)C[C@@H]2N(C)CC3)cc(C(C)=O)c1OC ; COc1cc([C@@]23C=C[C@H](O)C[C@@H]2N(C)CC3)cc(C(C)=O)c1OC]

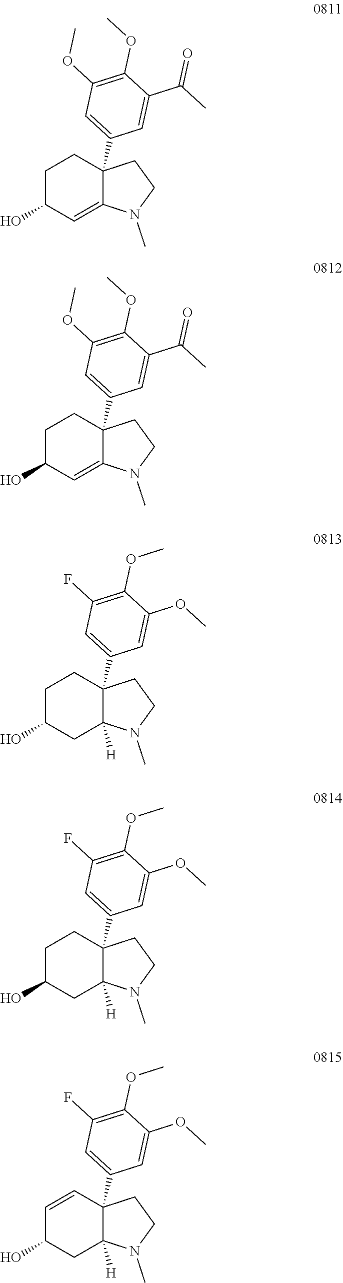 [SMILES: COc1cc([C@@]23CC[C@@H](O)C=C2N(C)CC3)cc(C(C)=O)c1OC ; COc1cc([C@@]23CC[C@H](O)C=C2N(C)CC3)cc(C(C)=O)c1OC ; COc1cc([C@@]23CC[C@@H](O)C[C@@H]2N(C)CC3)cc(F)c1OC ; COc1cc([C@@]23CC[C@H](O)C[C@@H]2N(C)CC3)cc(F)c1OC ; COc1cc([C@@]23C=C[C@@H](O)C[C@@H]2N(C)CC3)cc(F)c1OC]

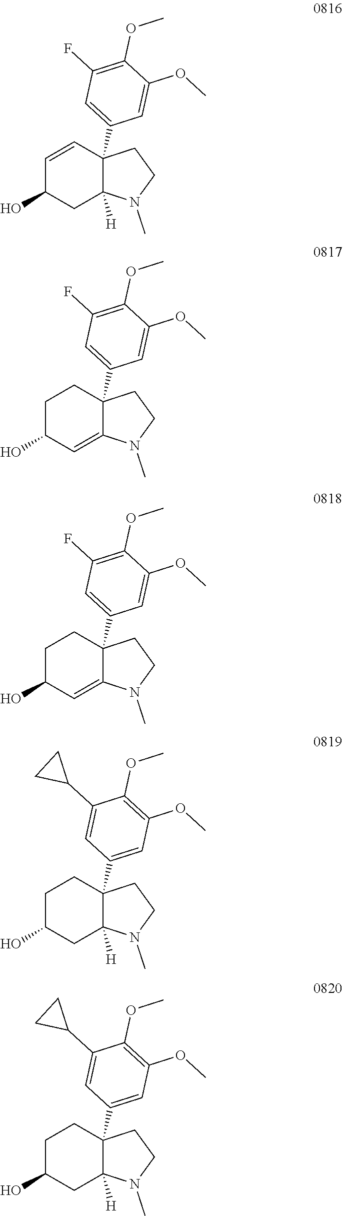

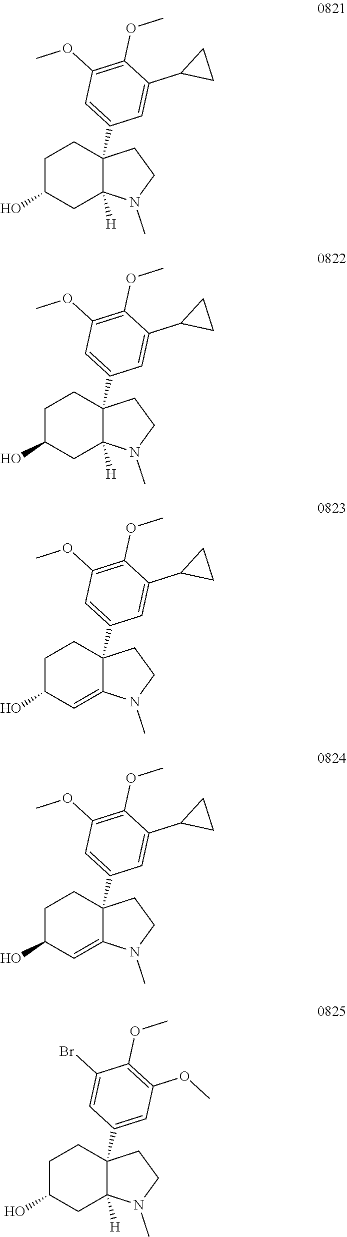

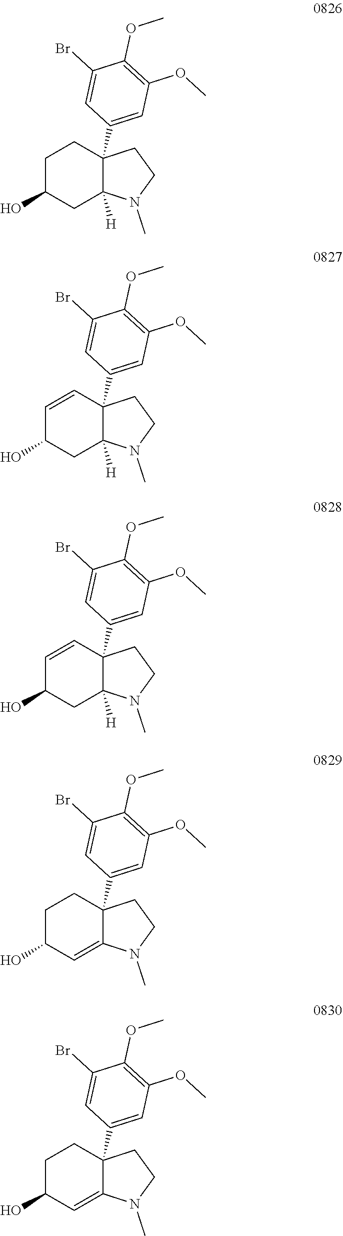

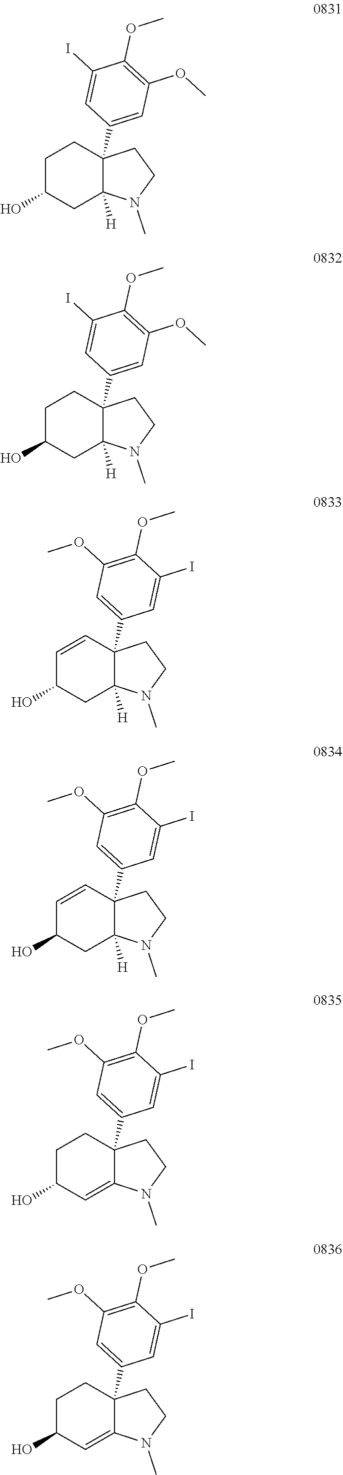

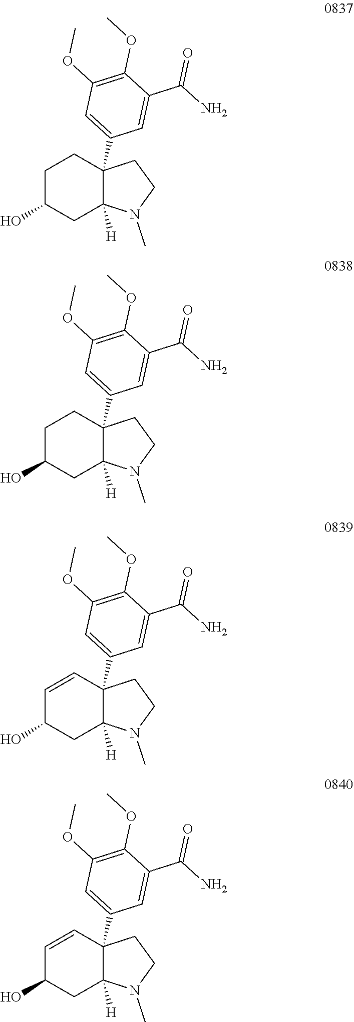

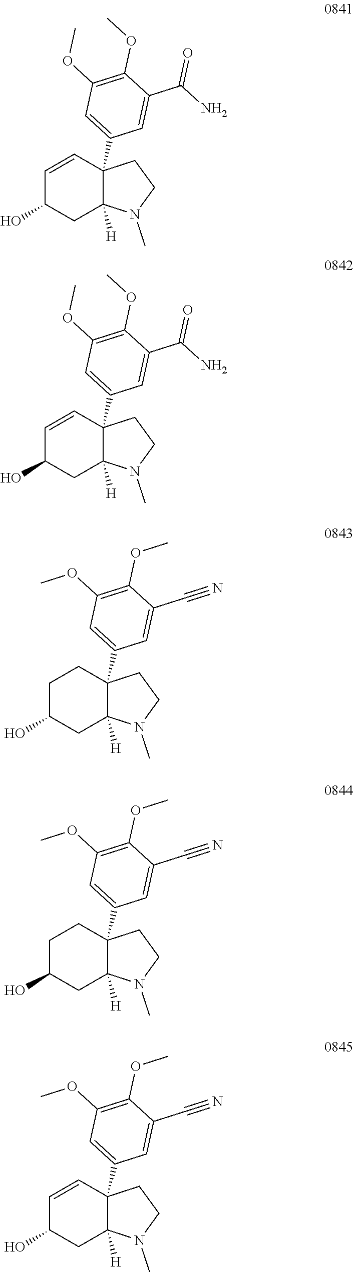

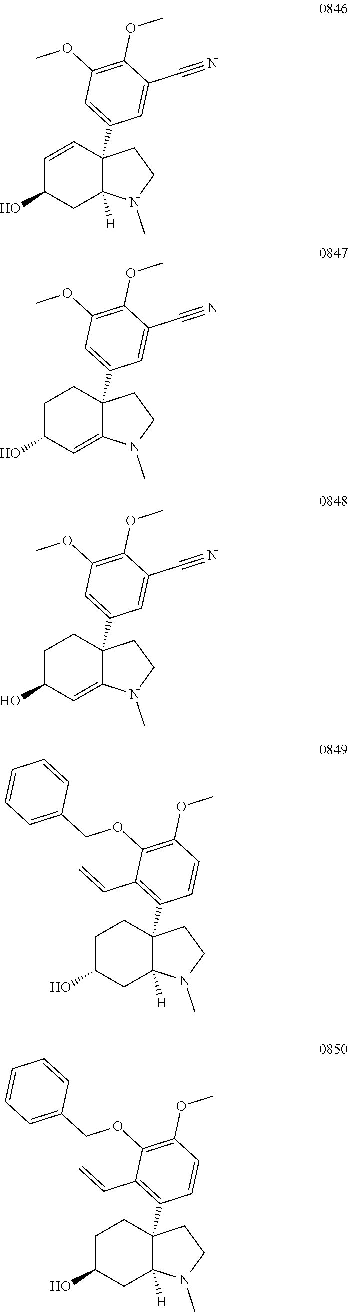

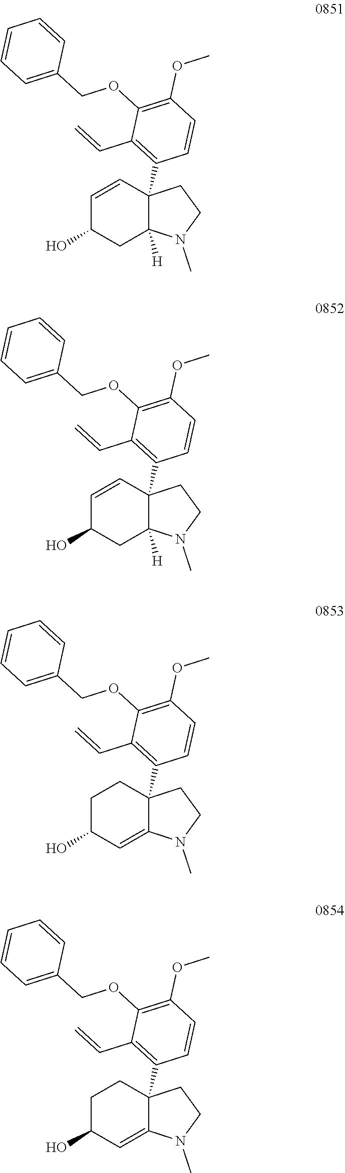 [SMILES: C=Cc1c([C@@]23C=C[C@@H](O)C[C@@H]2N(C)CC3)ccc(OC)c1OCc1ccccc1 ; C=Cc1c([C@@]23C=C[C@H](O)C[C@@H]2N(C)CC3)ccc(OC)c1OCc1ccccc1 ; C=Cc1c([C@@]23CC[C@@H](O)C=C2N(C)CC3)ccc(OC)c1OCc1ccccc1 ; C=Cc1c([C@@]23CC[C@H](O)C=C2N(C)CC3)ccc(OC)c1OCc1ccccc1]

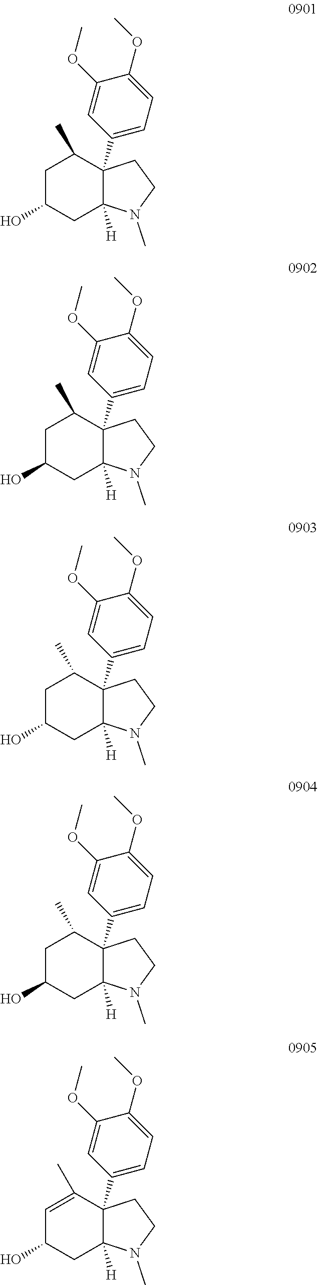

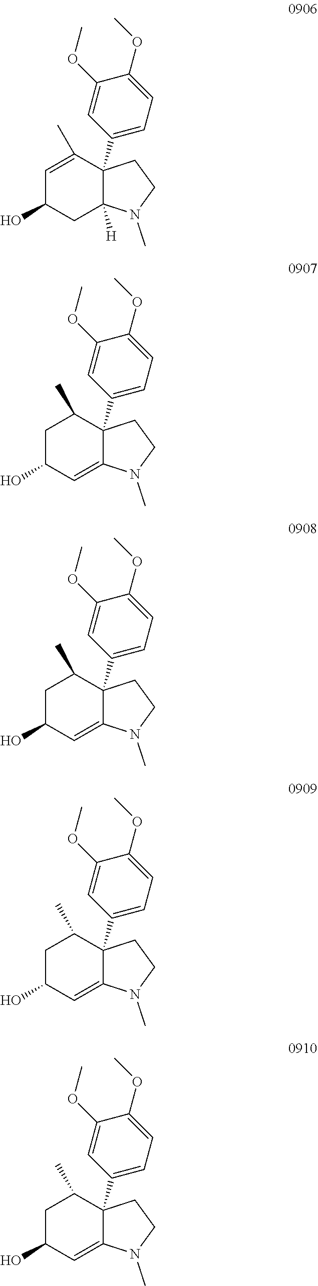

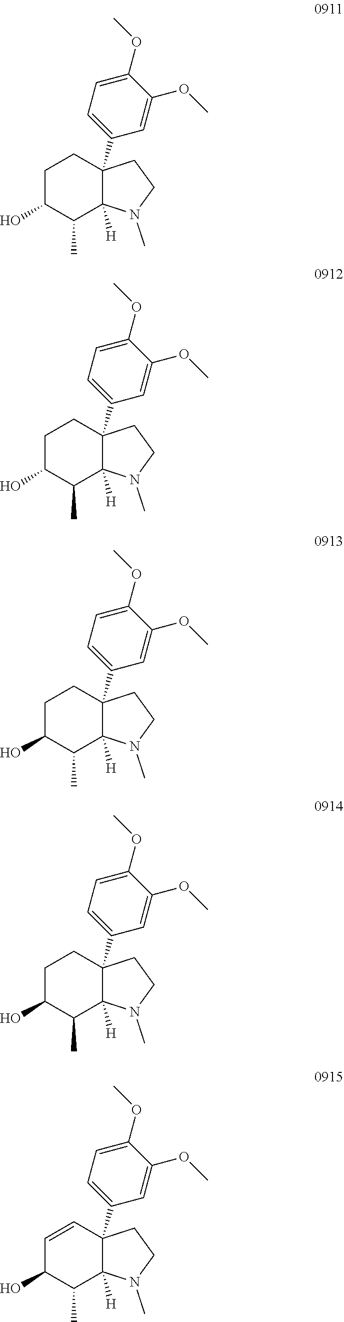 [SMILES: COc1ccc([C@@]23CC[C@@H](O)[C@@H](C)[C@@H]2N(C)CC3)cc1OC ; COc1ccc([C@@]23CC[C@@H](O)[C@H](C)[C@@H]2N(C)CC3)cc1OC ; COc1ccc([C@@]23CC[C@H](O)[C@@H](C)[C@@H]2N(C)CC3)cc1OC ; COc1ccc([C@@]23CC[C@H](O)[C@H](C)[C@@H]2N(C)CC3)cc1OC ; COc1ccc([C@@]23C=C[C@H](O)[C@@H](C)[C@@H]2N(C)CC3)cc1OC]

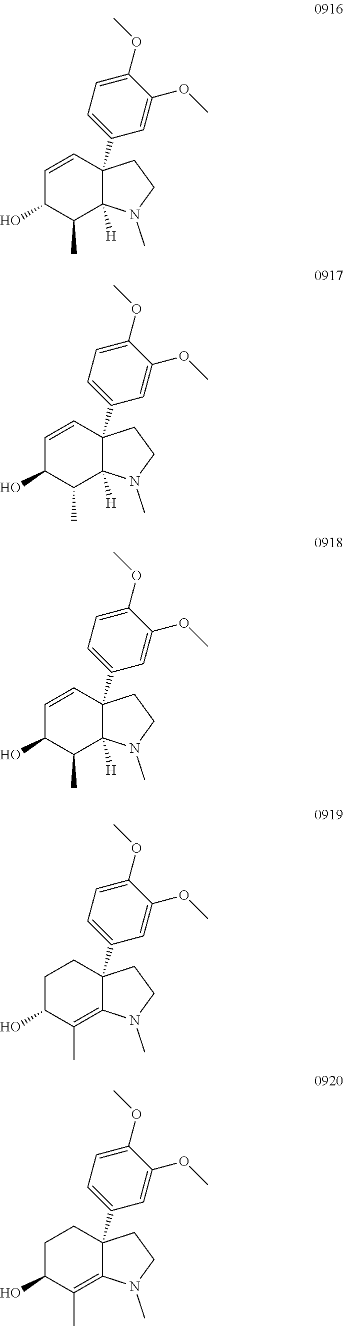 [SMILES: COc1ccc([C@@]23C=C[C@@H](O)[C@H](C)[C@@H]2N(C)CC3)cc1OC ; COc1ccc([C@@]23C=C[C@H](O)[C@@H](C)[C@@H]2N(C)CC3)cc1OC ; COc1ccc([C@@]23C=C[C@H](O)[C@H](C)[C@@H]2N(C)CC3)cc1OC ; COc1ccc([C@@]23CC[C@@H](O)C(C)=C2N(C)CC3)cc1OC ; COc1ccc([C@@]23CC[C@H](O)C(C)=C2N(C)CC3)cc1OC]

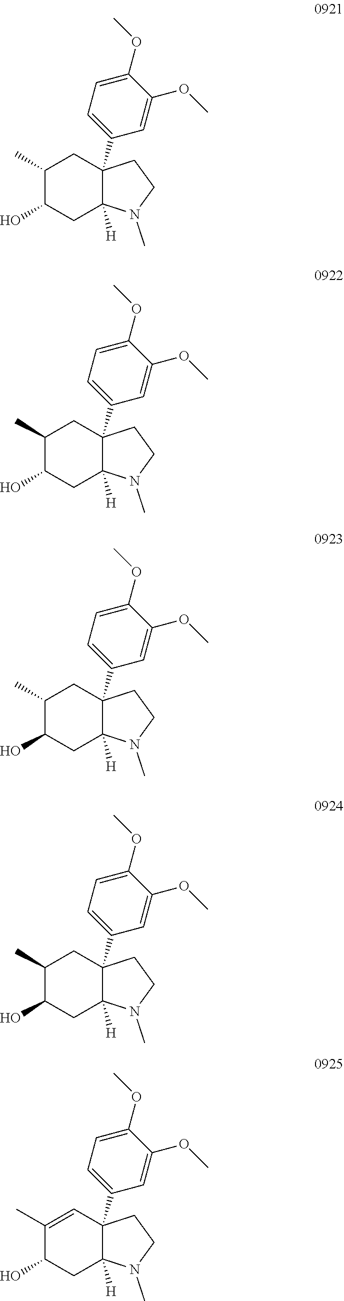

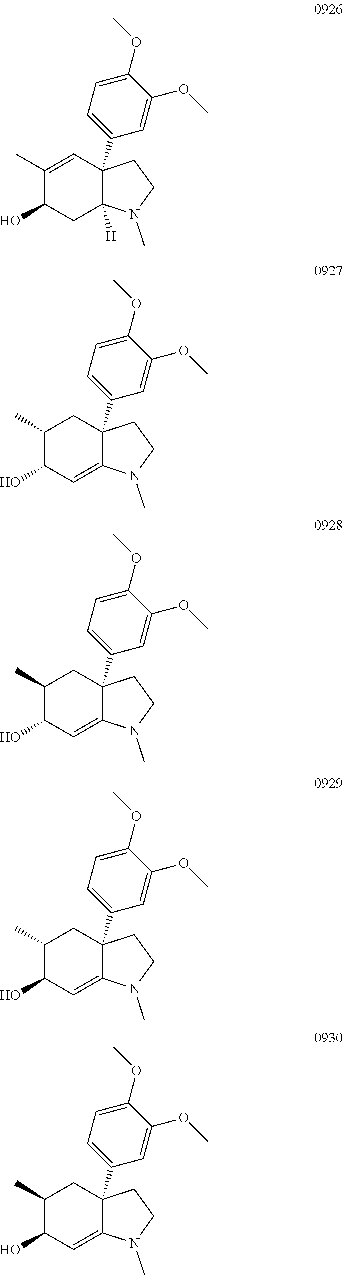

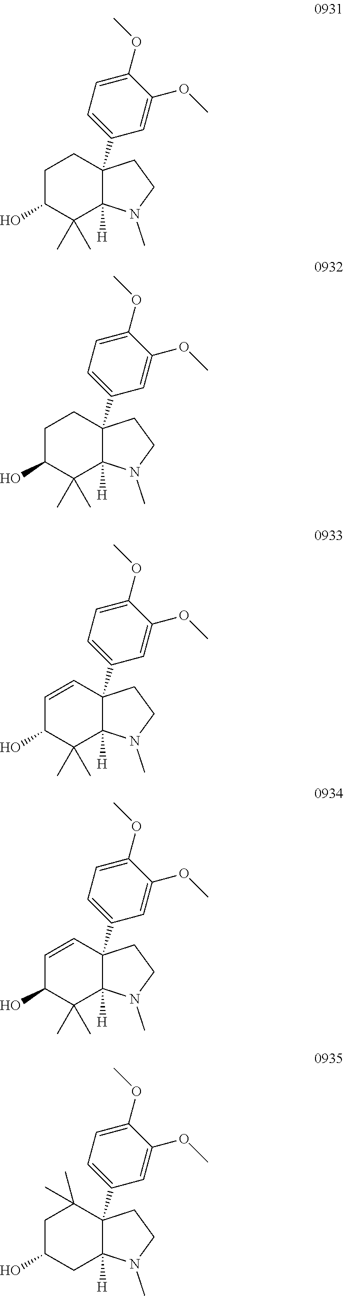

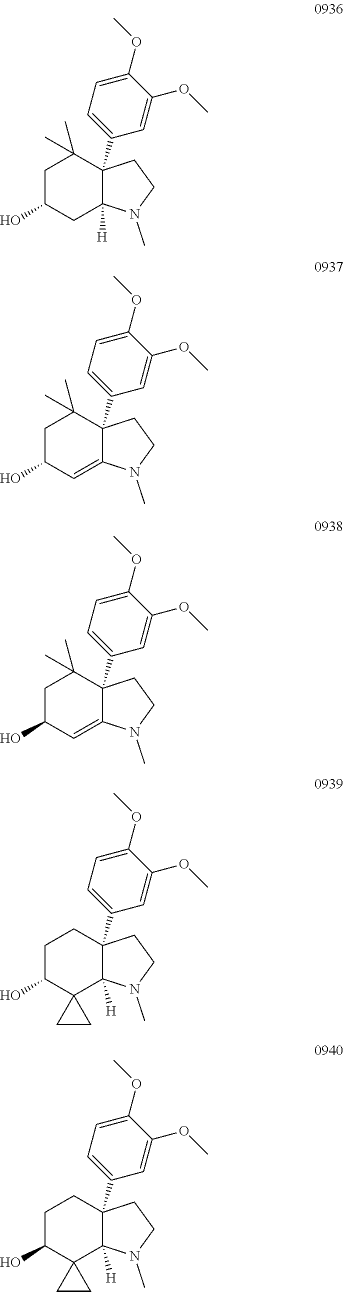

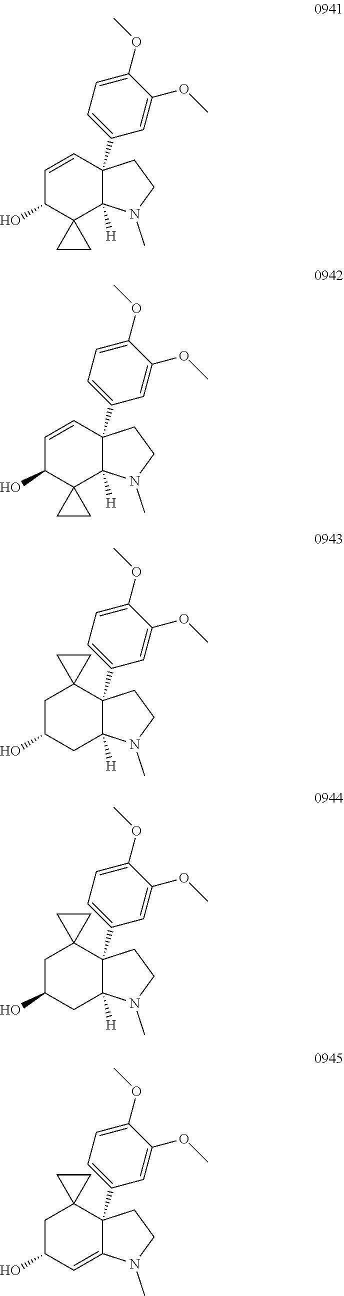

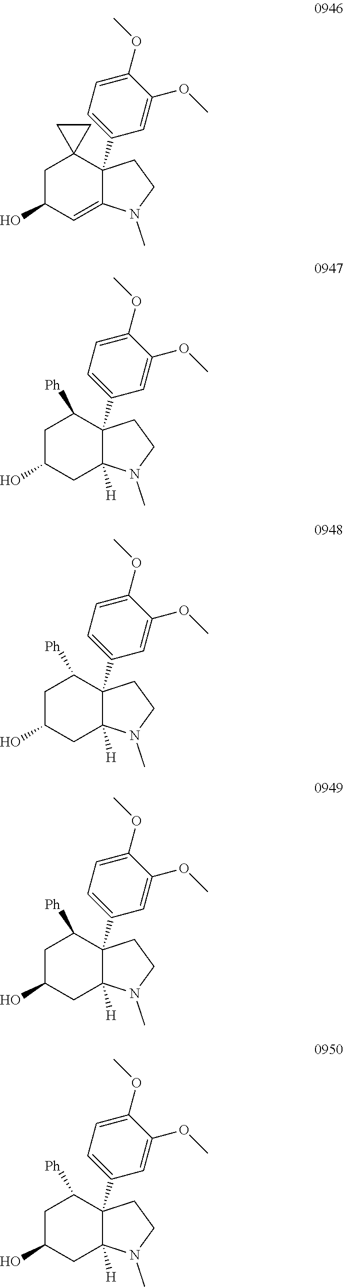

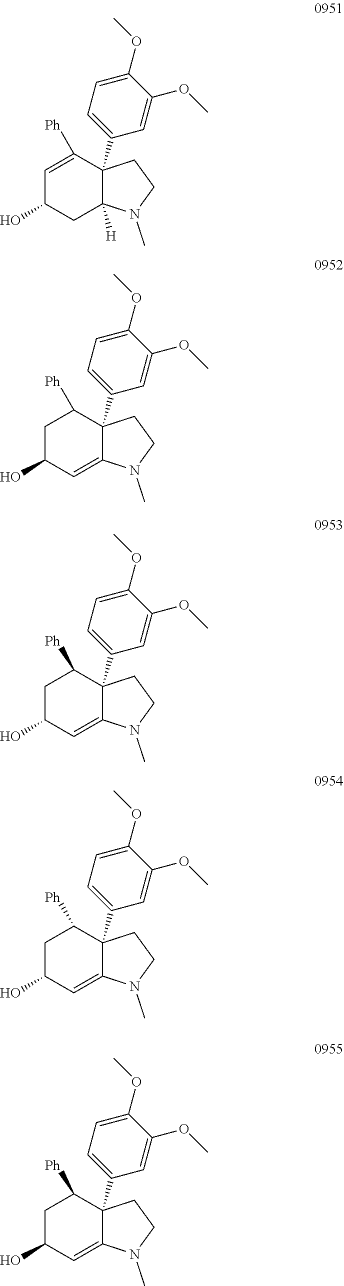

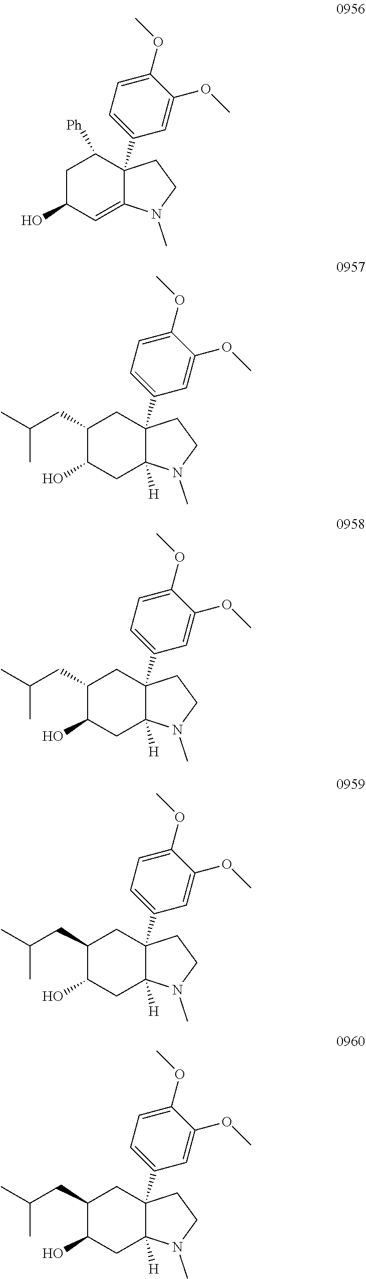

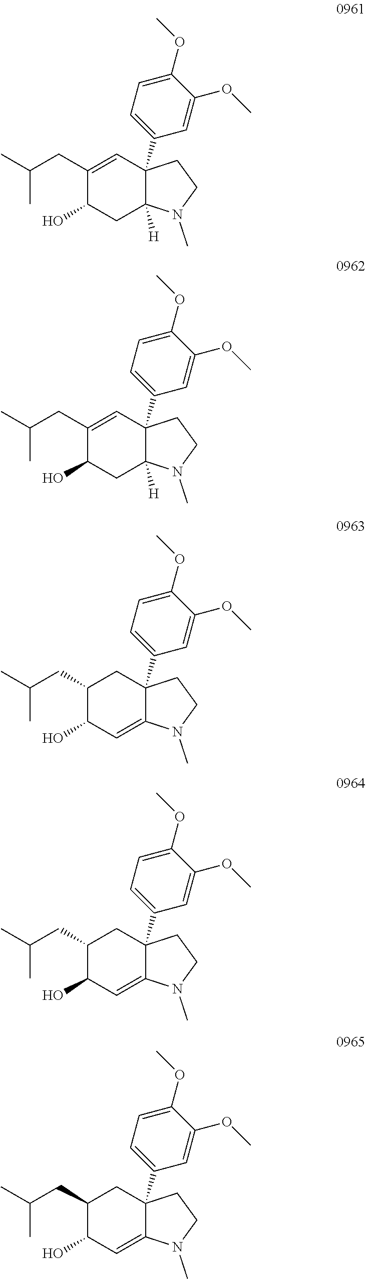

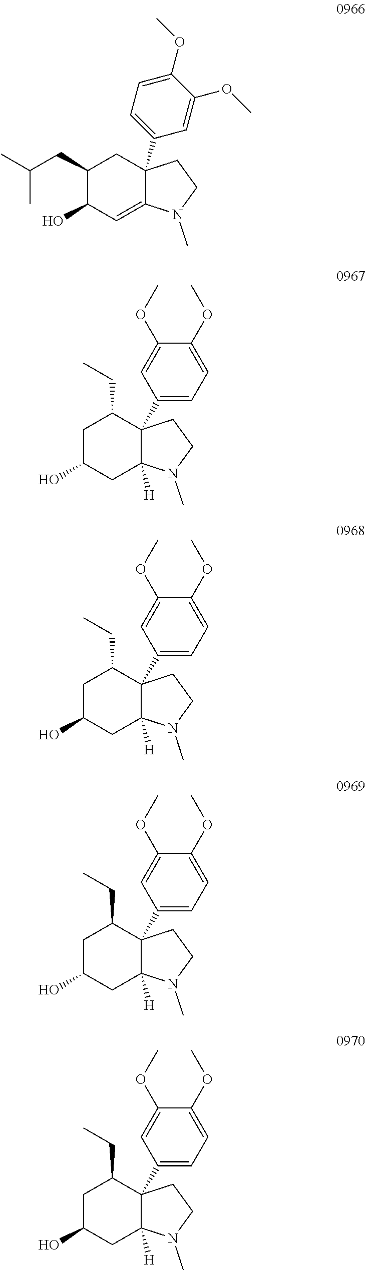

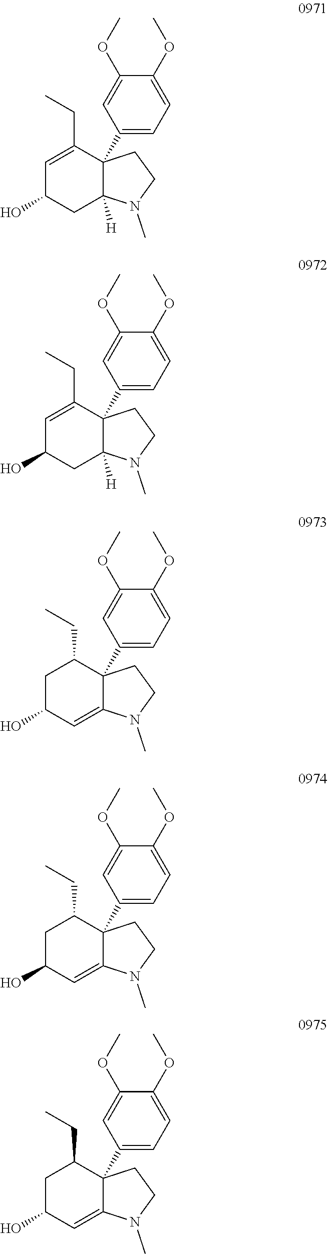

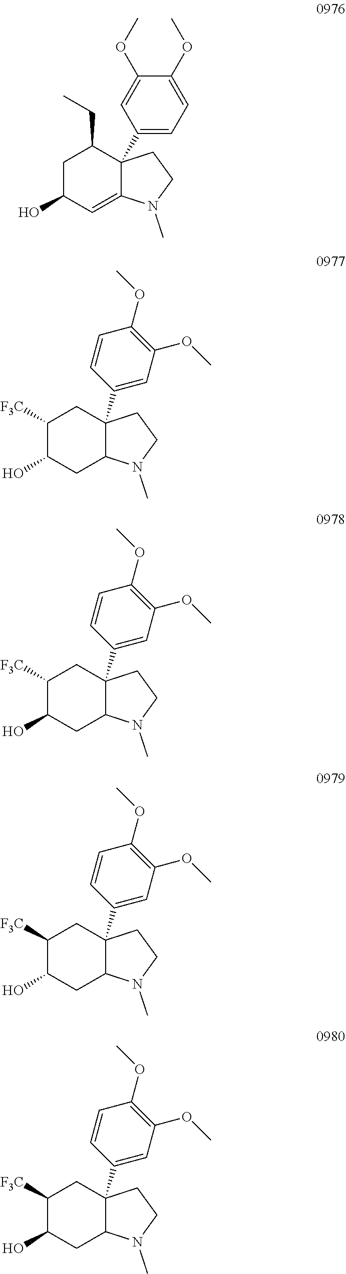

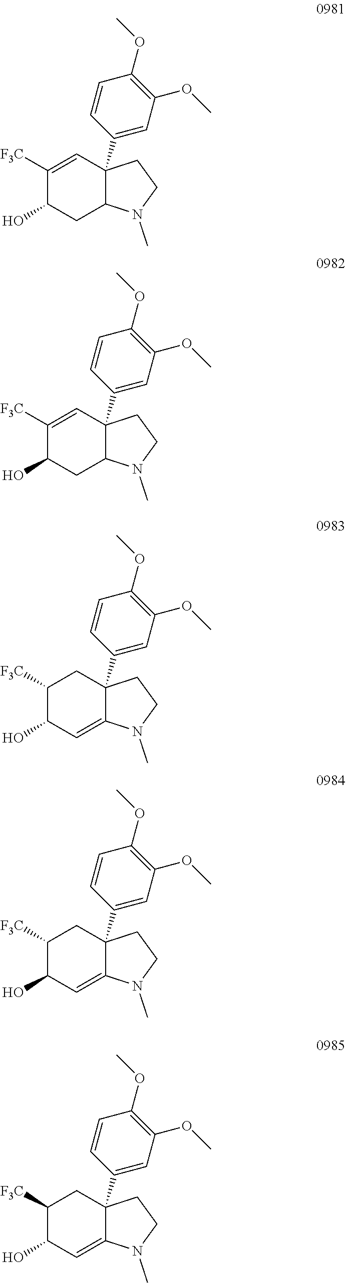

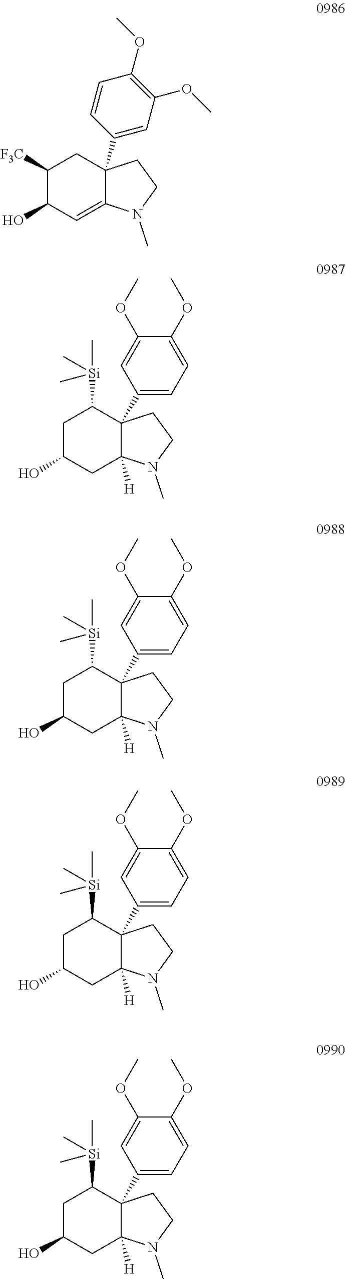

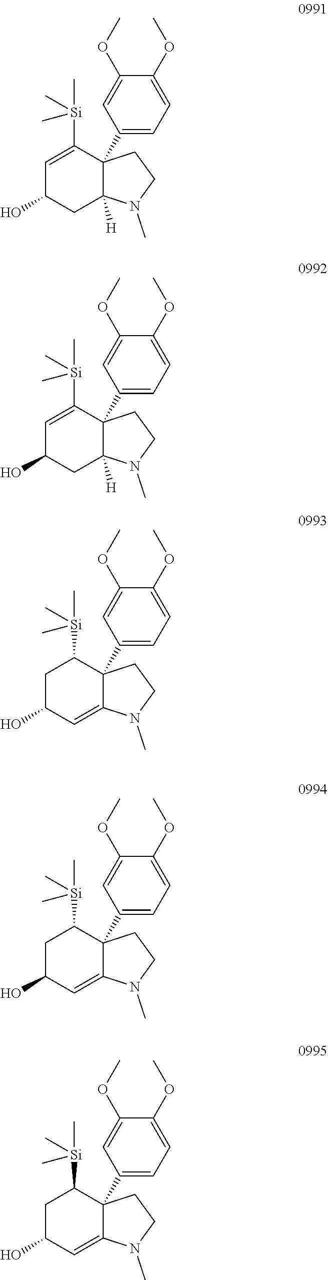

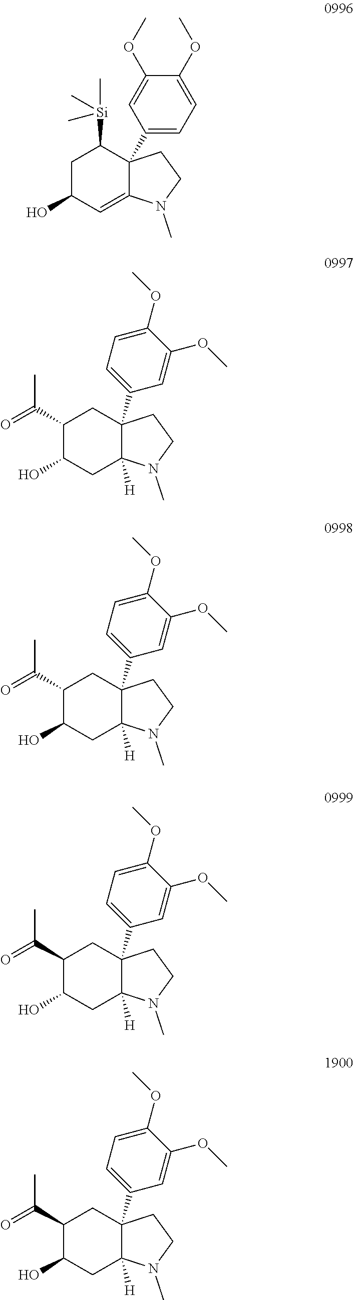 [SMILES: COc1ccc([C@]23CCN(C)C2=C[C@@H](O)C[C@H]3[Si](C)(C)C)cc1OC ; COc1ccc([C@]23CCN(C)[C@H]2C[C@H](O)[C@H](C(C)=O)C3)cc1OC ; COc1ccc([C@]23CCN(C)[C@H]2C[C@@H](O)[C@H](C(C)=O)C3)cc1OC ; COc1ccc([C@]23CCN(C)[C@H]2C[C@H](O)[C@@H](C(C)=O)C3)cc1OC ; COc1ccc([C@]23CCN(C)[C@H]2C[C@@H](O)[C@@H](C(C)=O)C3)cc1OC]

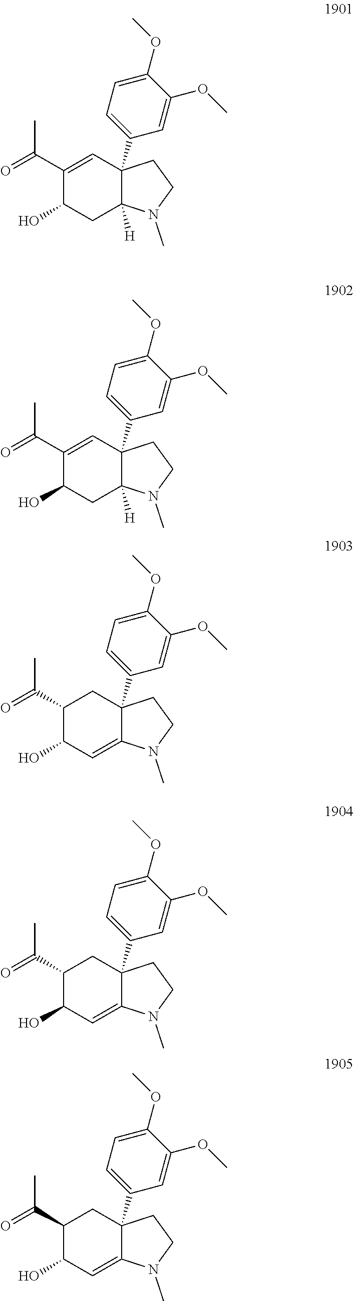

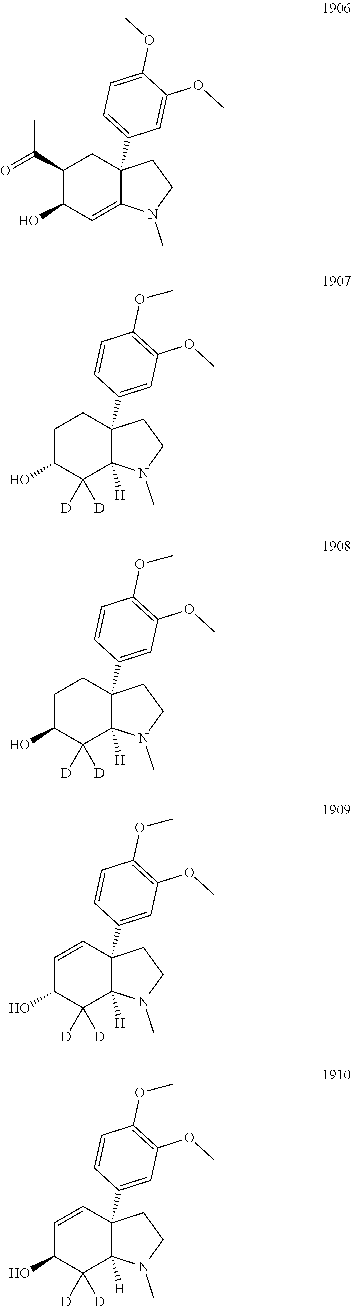

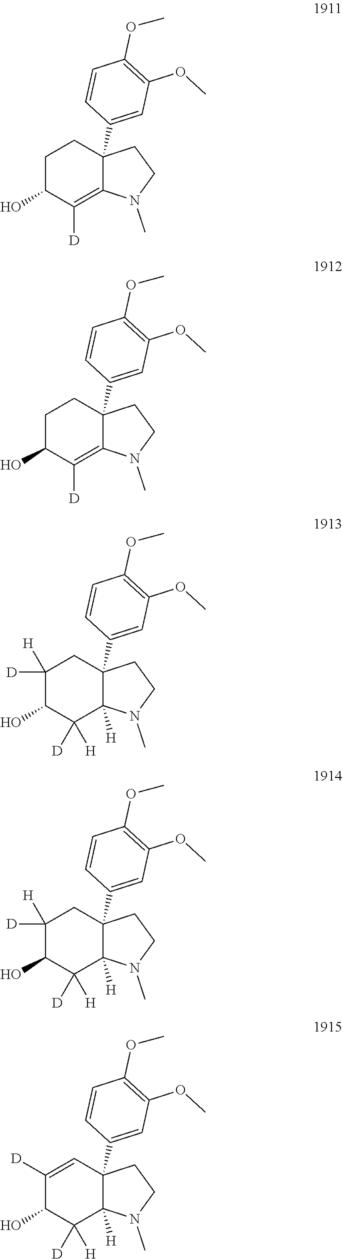

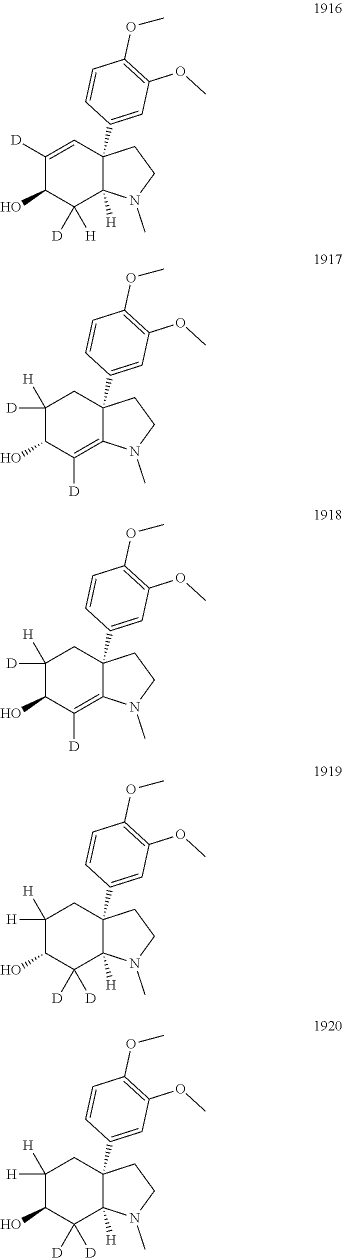

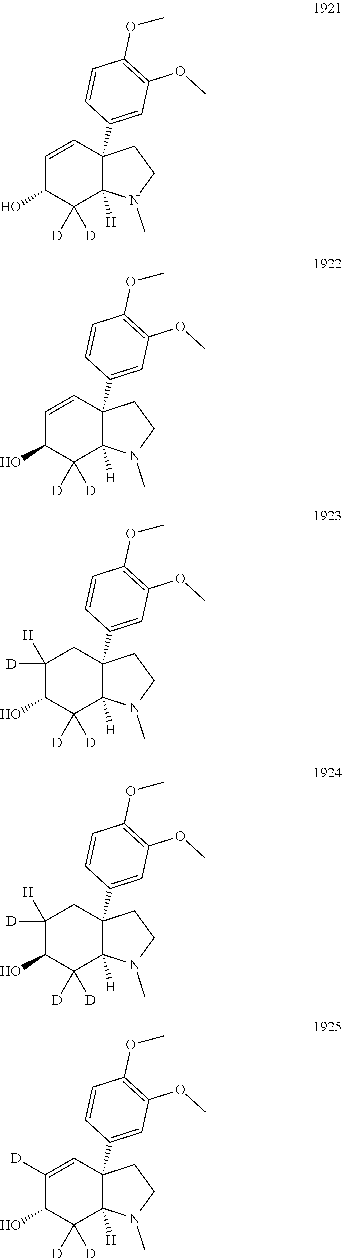

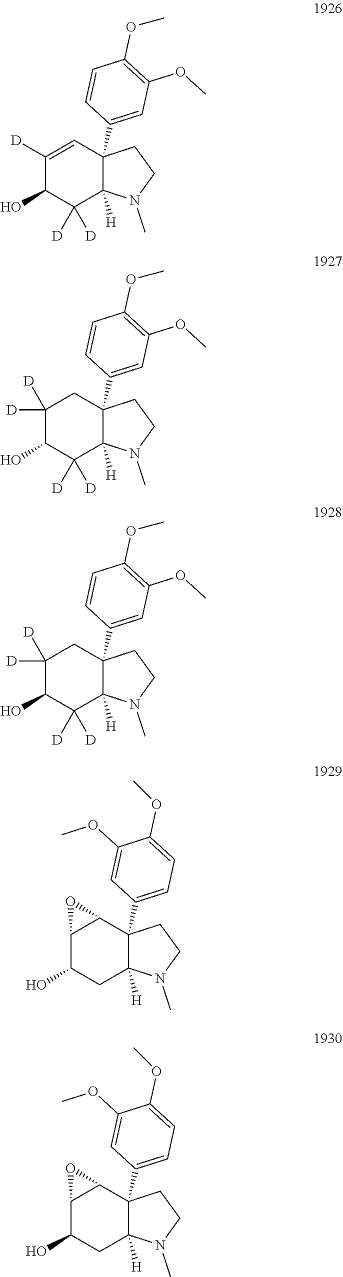

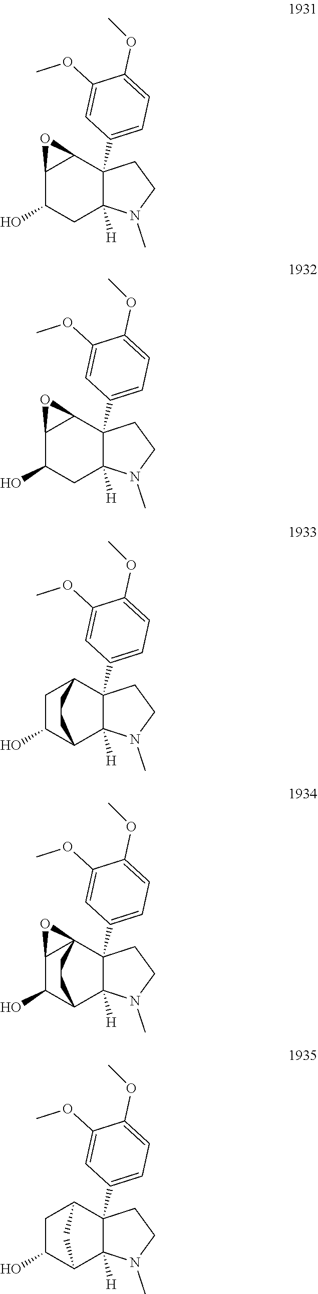

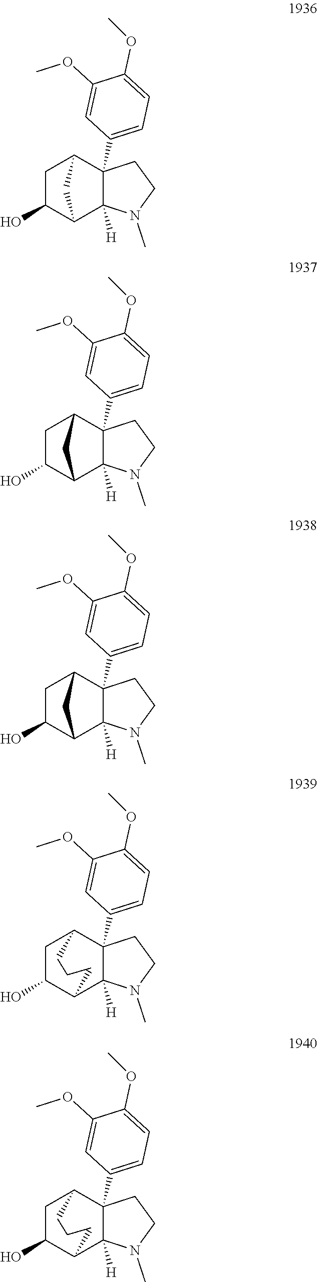

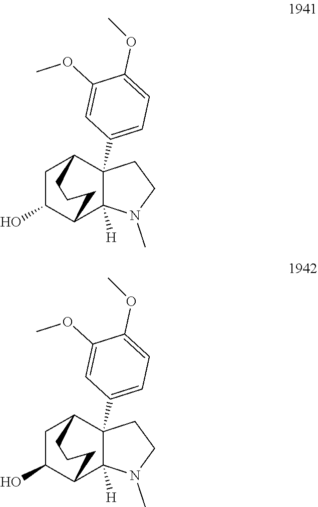

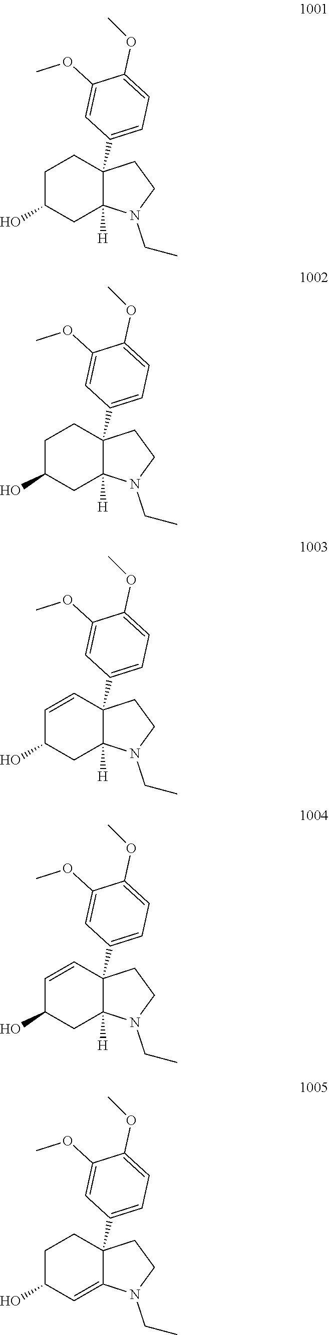 [SMILES: CCN1CC[C@]2(c3ccc(OC)c(OC)c3)CC[C@@H](O)C[C@H]12 ; CCN1CC[C@]2(c3ccc(OC)c(OC)c3)CC[C@H](O)C[C@H]12 ; CCN1CC[C@]2(c3ccc(OC)c(OC)c3)C=C[C@@H](O)C[C@H]12 ; CCN1CC[C@]2(c3ccc(OC)c(OC)c3)C=C[C@H](O)C[C@H]12 ; CCN1CC[C@]2(c3ccc(OC)c(OC)c3)CC[C@@H](O)C=C12]

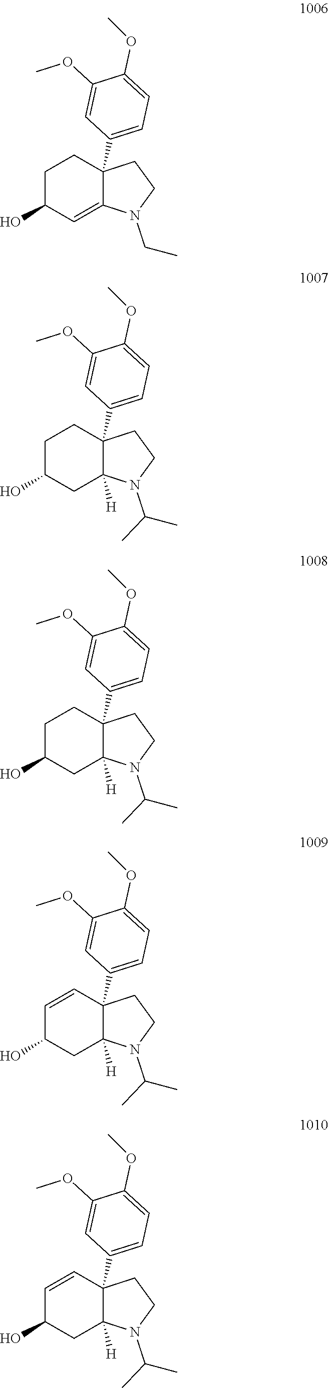

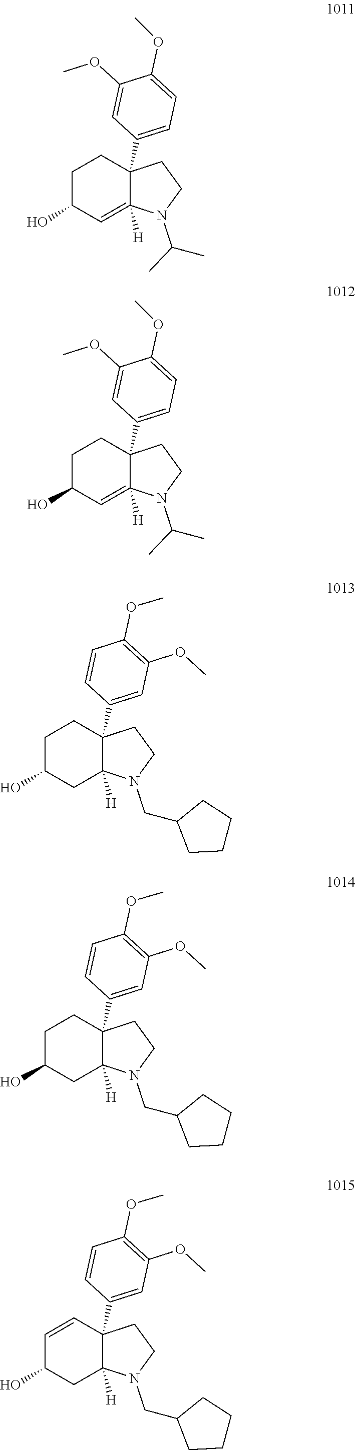

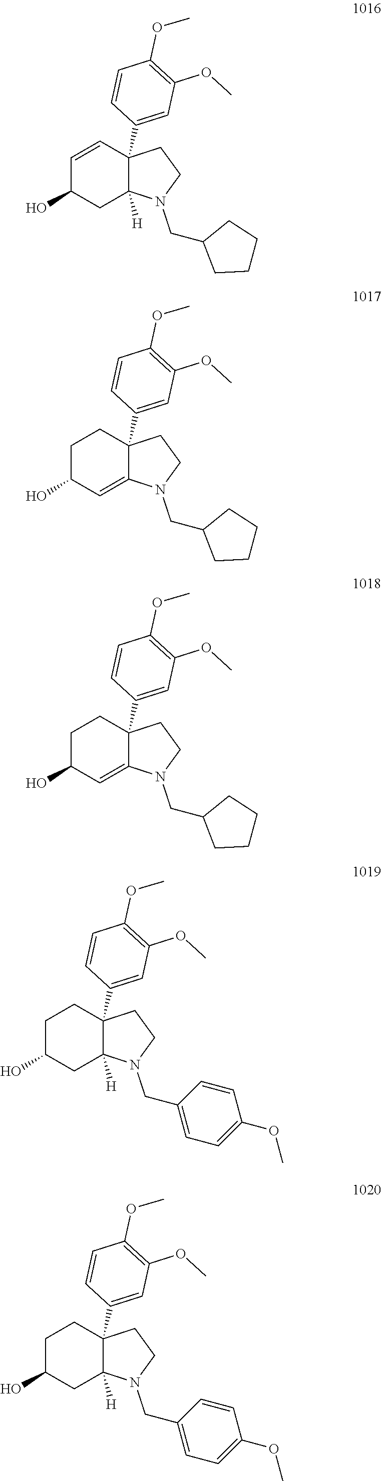

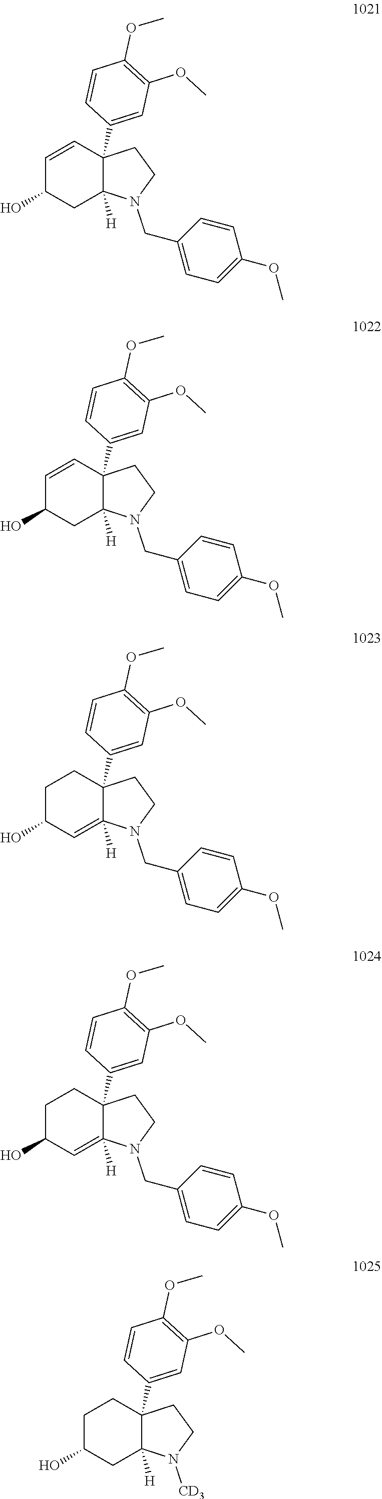

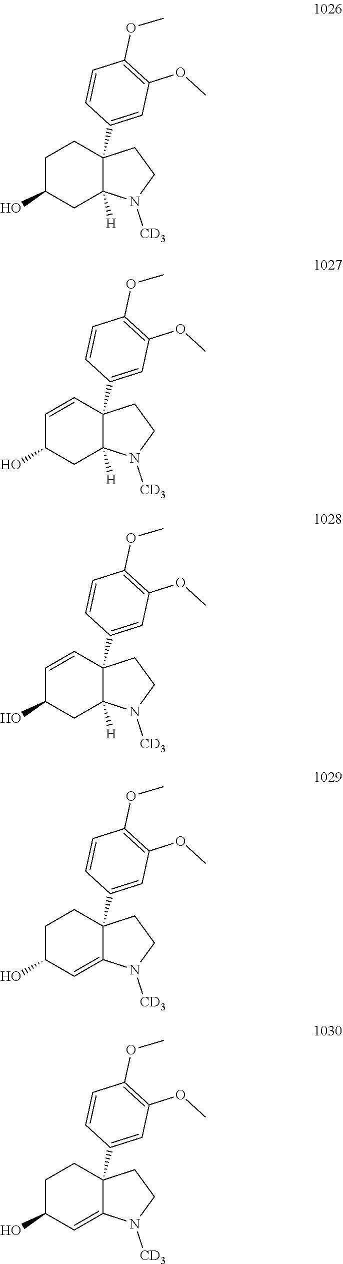

A-62. The compound of embodiment A-1, wherein the compound is

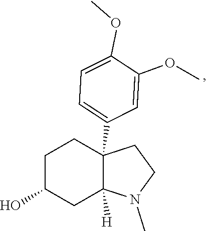 [SMILES: COc1ccc([C@@]23CC[C@@H](O)C[C@@H]2N(C)CC3)cc1OC]

or a pharmaceutically acceptable salt thereof.

A-63. The compound of embodiment A-1, wherein the compound is

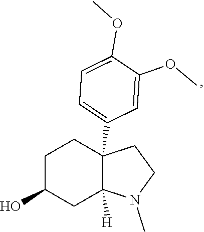 [SMILES: COc1ccc([C@@]23CC[C@H](O)C[C@@H]2N(C)CC3)cc1OC]

or a pharmaceutically acceptable salt thereof.

A-64. The compound of embodiment A-1, wherein the compound is

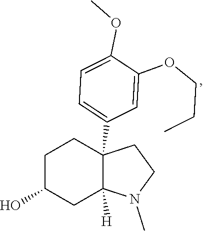 [SMILES: CCCOc1cc([C@@]23CC[C@@H](O)C[C@@H]2N(C)CC3)ccc1OC]

or a pharmaceutically acceptable salt thereof.

A-65. The compound of embodiment A-1, wherein the compound is

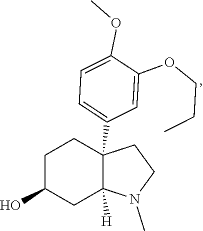 [SMILES: CCCOc1cc([C@@]23CC[C@H](O)C[C@@H]2N(C)CC3)ccc1OC]

or a pharmaceutically acceptable salt thereof.

A-66. The compound of embodiment A-1, wherein the compound is

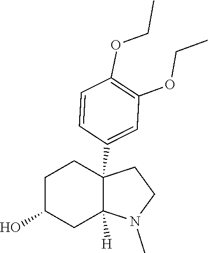 [SMILES: CCOc1ccc([C@@]23CC[C@@H](O)C[C@@H]2N(C)CC3)cc1OCC]

or a pharmaceutically acceptable salt thereof.

A-67. The compound of embodiment A-1, wherein the compound is

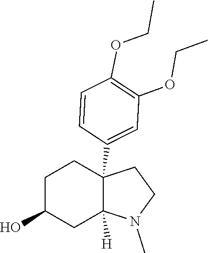 [SMILES: CCOc1ccc([C@@]23CC[C@H](O)C[C@@H]2N(C)CC3)cc1OCC]

or a pharmaceutically acceptable salt thereof.

A-68. The compound of embodiment A-1, wherein the compound is

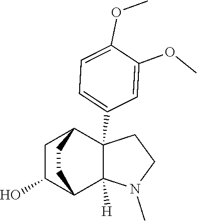

or a pharmaceutically acceptable salt thereof.

A-69. The compound of embodiment A-1, wherein the compound is

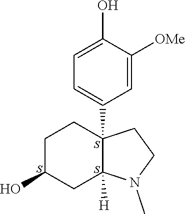

or a pharmaceutically acceptable salt thereof.

A-70. The compound of embodiment A-1, wherein the compound is

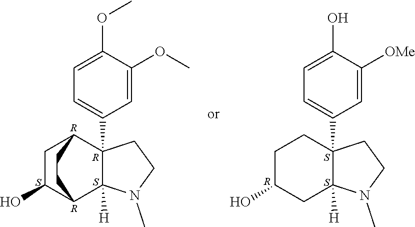

or a pharmaceutically acceptable salt thereof.

A-71. The compound of embodiment A-1, wherein the compound is

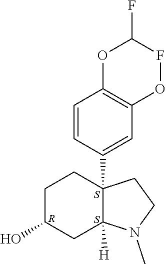

or a pharmaceutically acceptable salt thereof.

A-72. The compound of embodiment A-1, wherein the compound is

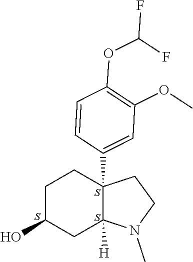

or a pharmaceutically acceptable salt thereof.

A-73. The compound of any one of embodiments A-1 to A-72, wherein the compound has the absolute stereochemistry shown.

A-74. A pharmaceutical composition, comprising a compound according to any one of embodiments A-1 to A-73; and a pharmaceutical acceptable excipient.

A-75. A method of treating a mental health disorder, comprising administering to a mammal in need thereof an effective amount of a compound according to any one of embodiments A-1 to A-74 or a pharmaceutically acceptable salt thereof.

A-76. The method of embodiment A-75, wherein the mental health disorder is anxiety, stress, or depression.

A-77. The method of embodiment A-75, wherein the mental health disorder is anxiety.

A-78. The method of embodiment A-75, wherein the mental health disorder is stress.

A-79. The method of embodiment A-75, wherein the mental health disorder is depression.

A-80. The method of any one of embodiments A-75 to A-79, wherein the mammal is a human.

### EXAMPLES

Certain compounds in the following examples are labeled using the MDL enhanced stereorepresentation. For example, the label “ABS” denotes the absolute stereochemistry at a particular stereocenter. The label “or n” or “orn” where n is an integer (e.g., “or1”), denotes a stereoisomer that has either the stereochemistry as drawn or is the epimer at that particular stereocenter. The label “and n” or “&n,” where n is an integer (e.g., “and 1” or “&1”) represents a mixture of two epimers at the stereocenter, i.e., the structure as drawn and the epimer in which the stereogenic center has the opposite configuration (e.g., a racemic mixture).

LC/MS spectra were obtained using Agilent 1200\G1956A or SHIMADZU LCMS-2020. Standard LC/MS conditions were as follows (running time 1.55 min):

Acidic condition: Mobile Phase A: 0.0375% TFA in water (v/v). Mobile Phase B: 0.01875% TFA in acetonitrile (v/v); Column: Kinetex EVO C18 30*2.1 mm, 5 μm.

Basic condition: Mobile Phase A: 0.025% NH₃·H₂O in water (v/v). Mobile Phase B: Acetonitrile; Column: Kinetex EVO C18 2.1×30 mm, 5 μm.

5-95AB_0.8 min

| Instrument | | SHIMADZU LCMS-2020; | | |
|---|---|---|---|---|
| Software | | LabSolution Version 5.97SP1 | | |
| HPLC | Column | Kinetex ® EVO C18 2.1 × 30 mm 5 um | | |
| | Mobile Phase | A: 0.0375% TFA in water (v/v) | | |
| | | B: 0.01875% TFA in Acetonitrile (v/v) | | |
| | Gradient | Time (min) | B (%) | Flow (mL/min) |
| | | 0.00 | 5.0 | 2.0 |
| | | 0.60 | 95.0 | 2.0 |
| | | 0.78 | 95.0 | 2.0 |
| | | 0.79 | 5.0 | 2.0 |
| | | 0.80 | 5.0 | 2.0 |

| | | |
|---|---|---|
| | Column Temp | 50° C. |
| | Detector | PDA (220 nm & 254 nm) |
| MS | Ionization source | ESI |
| | Drying Gas | N₂ |
| | Drying Gas Flow | 15 (L/min) |
| | DL Voltage | 120(V) |
| | Qarray DC Voltage | 20(V) |
| | MS Polarity | Positive |
| | MS Mode | Scan |
| | Mass range | 100-1000 |

Table of Abbreviations

| | |
|---|---|
| Ac | Acetyl |
| ACN | Acetonitrile |
| δ | Chemical shift |
| d | Doublet |
| DCM | Dichloromethane |
| DEA | Diethylamine |
| DIAD | Diisopropyl azodicarboxylate |
| DIBAL | Diisobutylaluminium hydride |
| DMA | Dimethylacetamide |
| DMF | N,N-Dimethylformamide |
| DMSO | Dimethyl sulfoxide |
| ESI | Electrospray ionization |
| Et | Ethyl |
| EtOAc | Ethyl acetate |
| FA | Formic Acid |
| HPLC | High Performance Liquid Chromatography |
| HMDS | Hexamethyldisilazide |
| hr | Hours |
| Hz | Hertz |
| IPA | Isopropanol |
| LC-MS | Liquid chromatography - mass spectrometry |
| LDA | Lithium diisopropylamide |
| m | Multiplet |
| m/z | Mass to charge ratio |
| Me | Methyl |
| min | Minutes |
| Ms | Methanesulfonyl |
| MS | Molecular sieves |
| NMR | Nuclear magnetic resonance |
| Py | Pyridine |
| rac- | racemic |
| s | Singlet (NMR) or seconds (time) |
| SFC | Supercritical fluid chromatography |
| t | Triplet |
| t-Bu | tert-Butyl |
| THF | Tetrahydrofuran |
| TMS | Trimethylsilyl |
| Tol | Toluene |
| Ts | p-Toluenesulfonyl |
| V | Volts |
| v/v | Volume per volume |
| Xantphos | 9,9-Dimethyl-4,5-bis(diphenylphosphino)xanthene |
| XPhos Pd G3 | (2-Dicyclohexylphosphino-2′,4′,6′-triisopropyl-1,1′- |
| | biphenyl)[2-(2′-amino-1,1′- |
| | biphenyl)]palladium(II) methanesulfonate |

### Example 1: Synthesis of (+/−)-Mesembrenone (0016) (+/−)-Mesembrine (0022)

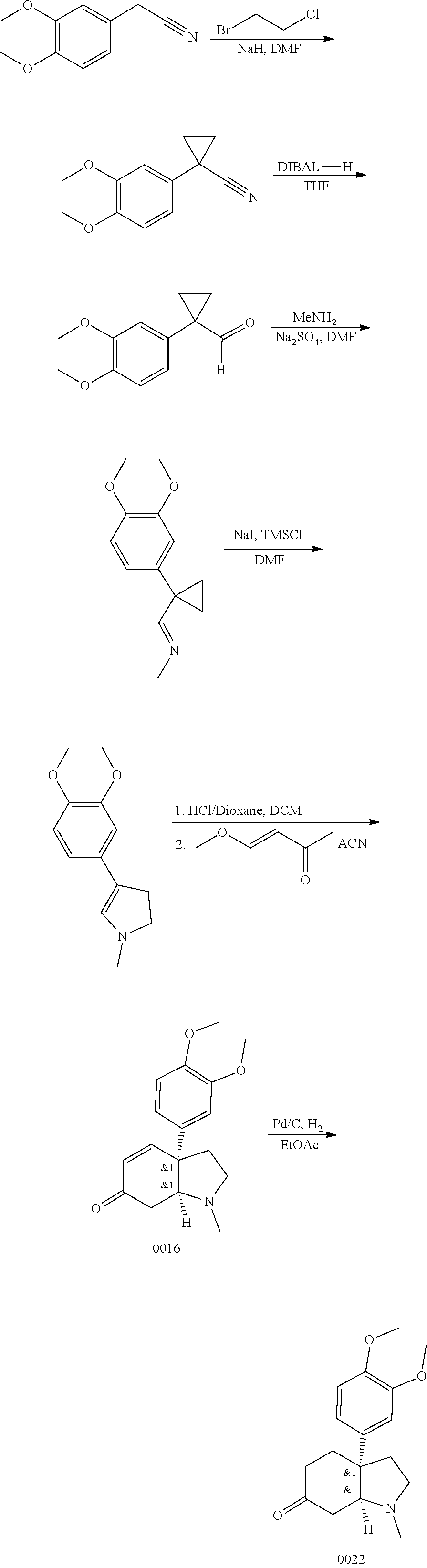

### Step 1: Synthesis of 1-(3,4-dimethoxyphenyl)cyclopropane-1-carbonitrile

To a solution of 2-(3,4-dimethoxyphenyl)acetonitrile (20 g, 112 mmol) in DMF (93 mL) was added NaH (18.0 g, 451 mmol, 60% purity) in portions. The mixture was allowed to stir at 25° C. for 20 min. 1-Bromo-2-chloro-ethane (16.1 g, 112 mmol) was added, and the mixture was allowed to stir at 25° C. for 16 hr. The reaction was quenched by the addition of a MeOH/water mixture (1:1, 1000 mL) and the resulting solution was extracted with EtOAc (3×500 mL). The organic solutions were combined, washed with water (4×500 mL) and brine (1×200 mL) and dried over (Na₂SO₄). The solution was filtered and the solvent was evaporated under reduced pressure. The resulting solid was purified by column chromatography (SiO₂, Petroleum ether/EtOAc=10/1 to 3/1) to give 1-(3,4-dimethoxyphenyl)cyclopropane-1-carbonitrile (15 g, 65%) as yellow oil. ¹H NMR (400 MHz, CDCl₃) δ 6.88 (s, 1H), 6.82 (d, J=1.2 Hz, 2H), 3.91 (s, 3H), 3.88 (s, 3H), 1.68-1.65 (m, 2H), 1.35 (d, J=2.4 Hz, 2H).

### Step 2: Synthesis of 1-(3,4-dimethoxyphenyl)cyclopropane-1-carbaldehyde

To a solution of 1-(3,4-dimethoxyphenyl)cyclopropane-1-carbonitrile (11 g, 54.1 mmol) in THE (160 mL) was added DIBAL-H (1M in toluene, 81.2 mL). The mixture was allowed to stir at 25° C. for 3 hr and then the reaction was cautiously quenched by addition of aqueous 2M HCl. The solution was extracted with DCM (3×200 mL). The organic solutions were combined, washed with water (2×200 mL) and brine (2×200 mL), and then dried over Na₂SO₄ to give 1-(3,4-dimethoxyphenyl)cyclopropane-1-carbaldehyde (9.6 g, 85%) as yellow oil. LC-MS (ESI+) m/z 207.0 (M+H)⁺. ¹H NMR (400 MHz, CDCl₃) δ 9.26 (s, 1H), 6.94-6.61 (m, 3H), 3.89 (d, J=2.8 Hz, 6H), 1.61-1.52 (m, 2H), 1.42-1.37 (m, 2H)

### Step 3: Synthesis of (Z)-1-(1-(3,4-dimethoxyphenyl)cyclopropyl)-N-methylmethanimine

To a solution of 1-(3,4-dimethoxyphenyl)-cyclopropanecarbaldehyde (5.0 g, 24.2 mmol) in DCM (50 mL) was added MeNH₂ (2 M, 121 mL) and Na₂SO₄ (15.5 g, 109 mmol, 11.0 mL). The mixture was allowed to stir at 25° C. for 16 hr. The reaction mixture was filtered and the filtrate was concentrated under reduced pressure to give (Z)-1-(1-(3,4-dimethoxyphenyl)cyclopropyl)-N-methylmethanimine (5.1 g, 99%) as white solid. LC-MS (ESI⁺) m/z 219.9 (M+H)⁺; ¹H NMR (400 MHz, CDCl₃) δ 7.55 (q, J=1.2 Hz, 1H), 6.93-6.77 (m, 3H), 3.88 (d, J=7.2 Hz, 6H), 3.24 (d, J=1.6 Hz, 3H), 1.29-1.23 (m, 2H), 1.18-1.12 (m, 2H).

### Step 4: Synthesis of 4-(3,4-dimethoxyphenyl)-1-methyl-2,3-dihydro-1H-pyrrole

To a solution of (Z)-1-(1-(3,4-dimethoxyphenyl)cyclopropyl)-N-methylmethanimine (5.4 g, 24.6 mmol) in DMF (19 mL) was added NaI (366 mg, 2.44 mmol) and TMSCl (267 mg, 2.46 mmol). The mixture was allowed to stir at 90° C. for 3 hr. The reaction mixture was diluted with water (100 mL) and extracted with EtOAc (100 mL×3). The organic solutions were combined, washed with water and brine, dried over Na₂SO₄, and filtered. The filtrate was concentrated under reduced pressure to give 4-(3,4-dimethoxyphenyl)-1-methyl-2,3-dihydro-1H-pyrrole (6.25 g, 80%) as yellow oil. LC-MS (ESI⁺) m/z 220.0 (M+H)⁺. ¹H NMR (400 MHz, CDCl₃) δ 6.90-6.66 (m, 3H), 6.31 (t, J=1.6 Hz, 1H), 3.95-3.80 (m, 6H), 3.18-3.11 (m, 2H), 2.79 (dt, J=1.2, 9.0 Hz, 2H), 2.65 (s, 3H).

### Step 5: Synthesis of rac-3a-(3,4-dimethoxyphenyl)-1-methyl-1,2,3,3a,7,7a-hexahydro-6H-indol-6-one (0016)

4-(3,4-Dimethoxyphenyl)-1-methyl-2,3-dihydro-1H-pyrrole (6.25 g, 28.5 mmol) was dissolved in DCM (100 mL). To this solution was added HCl (1M in dioxane, 25 mL, 100 mmol). The mixture was evaporated to dryness and then dissolved in ACN (90 mL). To this solution was added (E)-4-methoxybut-3-en-2-one (4.28 g, 42.7 mmol). The reaction mixture was allowed to stir at 90° C. for 16 hr. The reaction mixture was filtered and the filtrate was concentrated under reduced pressure. The residue was purified by HPLC (column: Phenomenex luna C18 (250*70 mm, 10 um); mobile phase: [water (NH₄HCO₃)-ACN]; B %: 22%-52%, 20 min). The eluant was acidified with aq. HCl to give 0016 (3.0 g, 30%) as a white solid. LC-MS (ESI⁺) m/z 288.3 (M+H)⁺. ¹H NMR (400 MHz, CDCl₃) δ 6.90-6.88 (m, 1H), 6.87-6.83 (m, 2H), 6.74 (dd, J=2.0, 10.1 Hz, 1H), 6.11 (d, J=10.0 Hz, 1H), 3.89 (d, J=4.0 Hz, 6H), 3.33 (dt, J=2.4, 8.8 Hz, 1H), 2.69-2.66 (m, 1H), 2.58-2.51 (m, 2H), 2.50-2.41 (m, 2H), 2.33 (s, 3H), 2.27-2.18 (m, 1H)

### Step 6: Synthesis of rac-3a-(3,4-dimethoxyphenyl)-1-methyloctahydro-6H-indol-6-one (0022)

A mixture of 0016 (12.0 g, 43.9 mmol) and 10% Pd/C (300 mg) in EtOAc (120 mL) was degassed and then purged with H₂ for 3 times. The mixture was allowed to stir at 25° C. for 2 hr under 15 psi H₂. The reaction mixture was filtered and the filtrate was concentrated in vacuo to give 0022 (10 g, 80%) as brown oil. LC-MS (ESI⁺) m/z 290.4 (M+H)⁺. ¹H NMR (400 MHz, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

Procedures similar to those described above in Example 1 were used to prepare ketones in the following table from the appropriate starting materials:

| Starting | | |
|---|---|---|
| Materials | Product | Characterization |
| 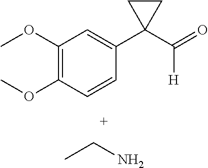 | 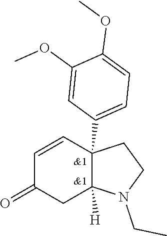 [SMILES: CCN1CC[C@]2(c3ccc(OC)c(OC)c3)C=CC(=O)C[C@H]12] | LC-MS (ESI⁺) m/z 302.1 (M + H)⁺; ¹H NMR (400 MHz, CDCl₃): δ 6.91 − 6.74 (m, 3H), 6.71 − 6.58 (m, 1H), 6.03 (d, J = 10.0 Hz, 1H), 3.81 (d, J = 3.6 Hz, 6H), 3.39 − 3.19 (m, 1H), 2.96 − 2.75 (m, 2H), 2.60 − 2.46 (m, 1H), 2.46 − 2.32 (m, 3H), 2.19 − 2.02 (m, 2H), 1.00 (t, J = 7.2 Hz, 3H). |
| | 1003-KR | |
| 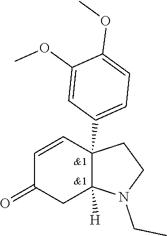 [SMILES: CCN1CC[C@]2(c3ccc(OC)c(OC)c3)C=CC(=O)C[C@H]12] | 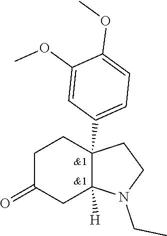 [SMILES: CCN1CC[C@]2(c3ccc(OC)c(OC)c3)CCC(=O)C[C@H]12] | LC-MS (ESI⁺) m/z 304.3 (M + H)⁺; ¹H NMR (400 MHz, CDCl₃): δ 7.09 − 6.77 (m, 3H), 3.90 (d, J = 7.2 Hz, 6H), 3.27 − 3.18 (m, 1H), 3.12 (t, J = 3.6 Hz, 1H), 2.95 − 2.82 (m, 1H), 2.59 (d, J = 3.6 Hz, 2H), 2.52 − 2.37 (m, 1H), 2.34 − 1.96 (m, 7H), 1.08 (t, J = 7.2 Hz, 3H). |
| 1003-KR | 1001-KR | |
| 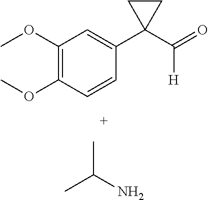 | 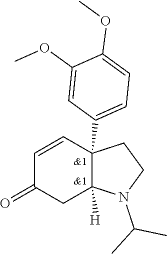 [SMILES: COc1ccc([C@@]23C=CC(=O)C[C@@H]2N(C(C)C)CC3)cc1OC] | LC-MS (ESI⁺) m/z 316.2 (M + H) ⁺; ¹H NMR (400 MHz, CDCl₃) δ 6.91 − 6.86 (m, 3H), 6.70 (dd, J = 2.0, 8.0 Hz, 1H), 6.13 (d, J = 10.0 Hz, 1H), 3.90 (s, 6H), 3.29 − 3.26 (m, 1H), 3.11 − 2.96 (m, 1H), 2.92 (dt, J = 5.2, 9.2 Hz, 1H), 2.57 − 2.48 (m, 2H), 2.45 − 2.31 (m, 1H), 2.16 − 2.05 (m, 2H), 1.09 (d, J = 6.8 Hz, 3H), 0.96 (d, J = 6.4 Hz, 3H). |
| | 1009-KR | |
| 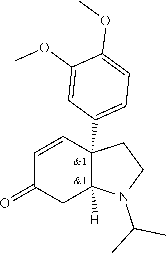 [SMILES: COc1ccc([C@@]23C=CC(=O)C[C@@H]2N(C(C)C)CC3)cc1OC] | 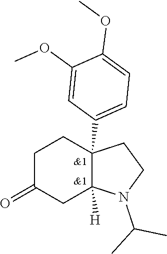 [SMILES: COc1ccc([C@@]23CCC(=O)C[C@@H]2N(C(C)C)CC3)cc1OC] | LC-MS (ESI⁺) m/z 318.3 (M + H) ⁺; ¹H NMR (400 MHz, CDCl₃) δ 6.94 (s, 2H), 6.86 (d, J = 8.0 Hz, 1H), 3.91 (d, J = 6.8 Hz, 6H), 3.53 (s, 1H), 3.13 (s, 1H), 3.05 − 2.87 (m, 1H), 2.69 (d, J = 8.4 Hz, 1H), 2.60 (s, 2H), 2.48 − 2.37 (m, 1H), 2.36 − 2.26 (m, 1H), 2.26 − 2.14 (m, 2H), 2.05 (d, J = 18.8 Hz, 2H), 1.15 (s, 3H), 0.96 (s, 3H). |
| 1009-KR | 1007-KR | |
| 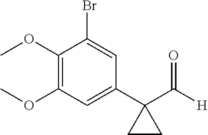 + Me—NH₂ | 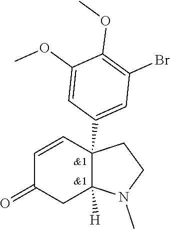 [SMILES: COc1cc([C@@]23C=CC(=O)C[C@@H]2N(C)CC3)cc(Br)c1OC] | LC-MS (ESI⁺) m/z 367.9 (M + H) ⁺ ¹H NMR (400 MHz, CDCl₃) 87.14 (d, J = 2.0 Hz, 1H), 6.83 (d, J = 2.0 Hz, 1H), 6.76 − 6.66 (m, 1H), 6.14 (d, J = 10.0 Hz, 1H), 3.89 (d, J = 5.6 Hz, 6H), 3.39 − 3.29 (m, 1H), 2.69 (s, 1H), 2.66 − 2.59 (m, 1H), 2.57 − 2.47 (m, 2H), 2.47 − 2.39 (m, 1H), 2.35 (s, 3H), 2.31 − 2.18 (m, 1H). |
| | 827-KR | |
| 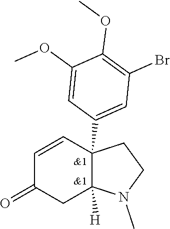 [SMILES: COc1cc([C@@]23C=CC(=O)C[C@@H]2N(C)CC3)cc(Br)c1OC] | 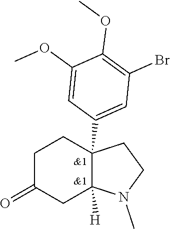 [SMILES: COc1cc([C@@]23CCC(=O)C[C@@H]2N(C)CC3)cc(Br)c1OC] | LC-MS (ESI⁺) m/z 368.0 (M + H) ⁺ |
| 827-KR | 825-KR | |
| 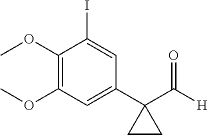 + Me NH₂ | 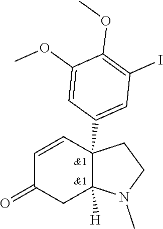 [SMILES: COc1cc([C@@]23C=CC(=O)C[C@@H]2N(C)CC3)cc(I)c1OC] | LC-MS (ESI⁺) m/z 413.9 (M + H) ⁺; ¹H NMR (400 MHz, CDCl₃) δ 7.32 (d, J = 2.0 Hz, 1H), 6.87 (d, J = 2.0 Hz, 1H), 6.71 (dd, J = 1.6, 10.0 Hz, 1H), 6.18 (d, J = 10.4 Hz, 1H), 3.87 (d, J = 11.2 Hz, 6H), 3.38 (dt, J = 3.2, 9.2 Hz, 1H), 2.85 (s, 1H), 2.71 − 2.60 (m, 2H), 2.55 (d, J = 4.0 Hz, 1H), 2.50 (dd, J = 3.6, 11.1 Hz, 1H), 2.40 (s, 3H), 2.32 − 2.19 (m, 1H). |
| | 833-KR | |
| 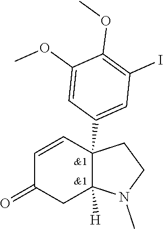 [SMILES: COc1cc([C@@]23C=CC(=O)C[C@@H]2N(C)CC3)cc(I)c1OC] | 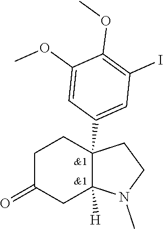 [SMILES: COc1cc([C@@]23CCC(=O)C[C@@H]2N(C)CC3)cc(I)c1OC] | LC-MS (ESI⁺) m/z 415.9 (M + H) ⁺; ¹H NMR (400 MHz, CDCl₃) δ 7.37 (d, J = 2.0 Hz, 1H), 6.90 (d, J = 2.0 Hz, 1H), 3.88 (d, J = 16.0 Hz, 6H), 3.21 − 3.08 (m, 1H), 2.93 (s, 1H), 2.61 (d, J = 3.2 Hz, 2H), 2.53 − 2.42 (m, 1H), 2.39 − 2.29 (m, 4H), 2.28 − 2.23 (m, 1H), 2.23 − 2.15 (m, 2H), 2.15 − 2.06 (m, 2H). |
| 833-KR | 831-KR | |
| 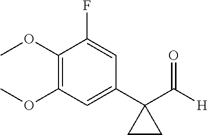 + Me NH₂ | 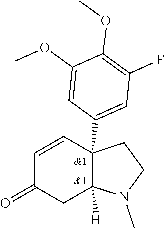 [SMILES: COc1cc([C@@]23C=CC(=O)C[C@@H]2N(C)CC3)cc(F)c1OC] | LC-MS (ESI⁺) m/z 306.1(M + H) ⁺ ¹H NMR (400 MHz, CDCl₃) δ 6.67 (dd, J = 2.2, 12.8 Hz, 1H), 6.63 − 6.59 (m, 1H), 6.58 (d, J = 1.6 Hz, 1H), 6.05 (d, J = 10.4 Hz, 1H), 3.86 (s, 3H), 3.81 (s, 3H), 3.24 (dt, J = 2.4, 8.8 Hz, 1H), 2.61 − 2.56 (m, 1H), 2.56 − 2.47 (m, 1H), 2.46 − 2.40 (m, 2H), 2.38 − 2.30 (m, 1H), 2.25 (s, 3H), 2.19 − 2.08 (m, 1H). |
| | 815-KR | |
| 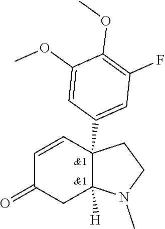 [SMILES: COc1cc([C@@]23C=CC(=O)C[C@@H]2N(C)CC3)cc(F)c1OC] | 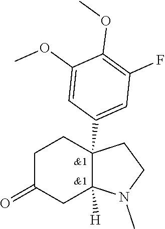 [SMILES: COc1cc([C@@]23CCC(=O)C[C@@H]2N(C)CC3)cc(F)c1OC] | LC-MS (ESI⁺) m/z 308.2 (M + H) ⁺ ¹H NMR (400 MHz, CDCl₃) δ = 6.83 − 6.67 (m, 1H), 6.62 (s, ^{1H)} 4.07 − 3.69 (m, 6H), 3.15 − 2.95 (m, 1H), 2.91 − 2.80 (m, 1H), 2.52 (d, J = 2.8 Hz, 2H), 2.45 − 2.33 (m, 1H), 2.31 − 2.21 (m, 4H), 2.18 − 1.93 (m, 5H). |
| 815-KR | 813-KR | |
| 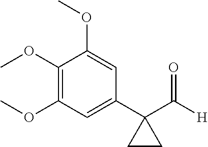 + Me NH₂ | 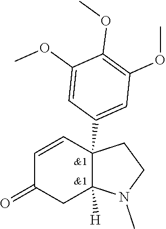 [SMILES: COc1cc([C@@]23C=CC(=O)C[C@@H]2N(C)CC3)cc(OC)c1OC] | LC-MS (ESI⁺) m/z 317.9 (M + H)⁺. ¹H NMR (400 MHz, CDCl₃) δ 6.62 (dd, J = 2.0, 10.1 Hz, 1H), 6.45 (s, 2H), 6.02 (d, J = 10.0 Hz, 1H), 3.84 − 3.69 (m, 9H), 3.29 − 3.17 (m, 1H), 2.58 (br s, 1H), 2.50 − 2.43 (m, 2H), 2.42 − 2.32 (m, 2H), 2.23 (s, 3H), 2.12 (td, J = 8.4, 13.0 Hz, 1H). |
| | 803-KR | |
| 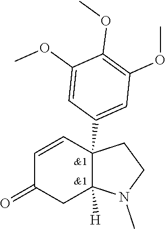 [SMILES: COc1cc([C@@]23C=CC(=O)C[C@@H]2N(C)CC3)cc(OC)c1OC] | 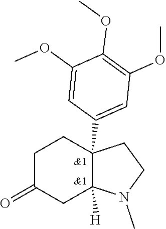 [SMILES: COc1cc([C@@]23CCC(=O)C[C@@H]2N(C)CC3)cc(OC)c1OC] | LC-MS (ESI⁺) m/z 320.2 (M + H)+. ¹H NMR (400 MHz, CDCl₃) δ 6.60 (s, 2H), 3.95 − 3.81 (m, 9H), 3.18 − 3.11 (m, 1H), 2.95 (t, J = 3.2 Hz, 1H), 2.62 (d, J = 3.6 Hz, 2H), 2.52 − 2.41 (m, 1H), 2.38 − 2.29 (m, 4H), 2.25 (s, 1H), 2.23 − 2.19 (m, 2H), 2.18 − 2.10 (m, 2H). |
| 803-KR | 801-KR | |
| 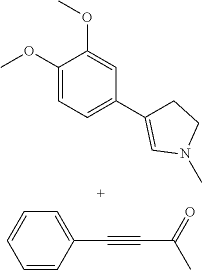 | 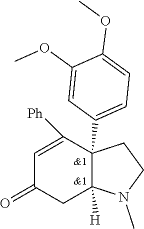 [SMILES: COc1ccc([C@]23CCN(C)[C@H]2CC(=O)C=C3c2ccccc2)cc1OC] 951-KR | LC-MS (ESI⁺) m/z 364.0 (M + H)⁺. ¹H NMR (400 MHz, CDCl₃) δ = 7.82 (s, 1H), 7.13 − 6.98 (m, 5H), 6.74 − 6.60 (m, 2H), 6.36 (s, 1H), 3.89 (d, J = 7.6 Hz, 2H), 3.83 (d, J = 0.4 Hz, 3H), 3.52 − 3.45 (m, 5H), 3.17 (s, 3H), 2.90 − 2.84 (m, 2H). |
| 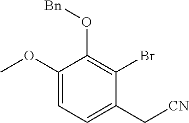 | 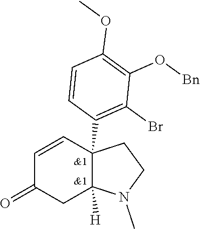 [SMILES: COc1ccc([C@@]23C=CC(=O)C[C@@H]2N(C)CC3)c(Br)c1OCc1ccccc1] | LC-MS (ESI⁺) m/z 443.7 (M + H)⁺. ¹H NMR (400 MHz, CDCl₃) δ 7.56 (d, J = 7.2 Hz, 2H), 7.43 − 7.32 (m, 3H), 7.14 ( d, J = 2.0 Hz, 1H), 6.91 − 6.81 (m, 1H), 6.70 (dd, J = 1.6, 10.0 Hz, 1H), 6.14 (d, J = 10.0 Hz, 1H), 5.08 − 4.99 (m, 2H), 3.88 (s, 3H), 3.44 − 3.29 (m, 1H), 3.06 (s, 1H), 2.99 − 2.83 (m, 2H), 2.72 − 2.44 (m, 4H), 2.31 − 2.21 (m, 2H). |
| | 85-KR | |
| 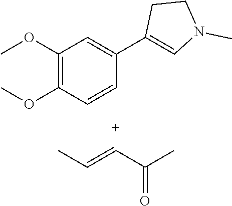 | 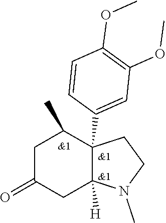 [SMILES: COc1ccc([C@]23CCN(C)[C@H]2CC(=O)C[C@H]3C)cc1OC] | LC-MS (ESI⁺) m/z 304.3 (M + H)⁺. |
| | 901-KR | |
| 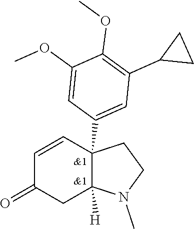 [SMILES: COc1cc([C@@]23C=CC(=O)C[C@@H]2N(C)CC3)cc(C2CC2)c1OC] | 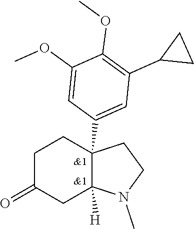 [SMILES: COc1cc([C@@]23CCC(=O)C[C@@H]2N(C)CC3)cc(C2CC2)c1OC] | LC-MS (ESI⁺) m/z 330.4 (M + H)⁺. ¹H NMR (400 MHz, CDCl₃) δ 6.74 (d, J = 2.0 Hz, 1H), 6.42 (d, J = 2.0 Hz, 1H), 3.89 (d, J = 5.1 Hz, 6H), 3.19 − 3.10 (m, 1H), 2.93 (br s, 1H), 2.61 (d, J = 3.6 Hz, 2H), 2.53 − 2.37 (m, 2H), 2.33 (s, 3H), 2.31 − 2.22 (m, 2H), 2.21 − 2.04 (m, 4H), 1.05 − 0.96 (m, 2H), 0.72 − 0.64 (m, 2H). |
| 821-KR | 819-KR | |
| 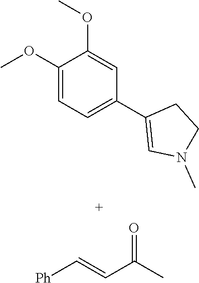 [SMILES: COc1ccc(C2=CN(C)CC2)cc1OC ; CC(=O)/C=C/c1ccccc1] | 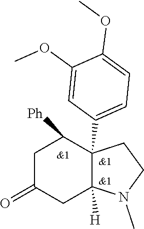 [SMILES: COc1ccc([C@]23CCN(C)[C@H]2CC(=O)C[C@H]3c2ccccc2)cc1OC] 947-KR | LC-MS (ESI⁺) m/z 366.4 (M + H)⁺. |
| 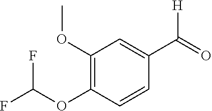 + MeNH₂ | 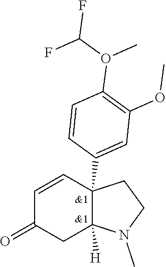 | LC-MS (ESI⁺) m/z 323.9 (M + H)⁺. |
| | 2725-KR | |
| 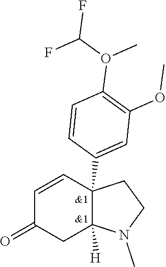 | 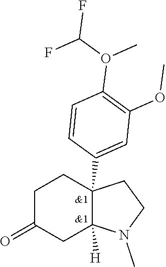 | LC-MS (ESI⁺) m/z 326.2 (M + H)⁺. ¹H NMR (400 MHz, CDCl₃) δ 7.15 (d, J = 8.4 Hz, 1H), 7.02 − 6.92 (m, 2H), 6.56 (t, J = 76 Hz, 1H), 3.99 − 3.84 (m, 3H), 3.80 − 3.69 (m, 1H), 3.22 − 3.10 (m, 1H), 2.95 (t, J = 3.2 Hz, 1H), 2.61 (d, J = 3.6 Hz, 2H), 2.49 − 2.44 (m, 1H), 2.36 − 2.31 (m, 3H), 2.25 − 2.10 (m, 4H), 1.90 − 1.83 (m, 1H). |
| 2725-KR | 2723-KR | |
| 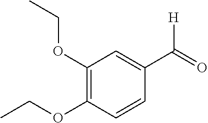 + MeNH₂ | 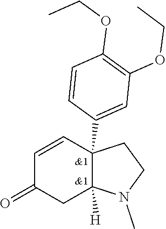 [SMILES: CCOc1ccc([C@@]23C=CC(=O)C[C@@H]2N(C)CC3)cc1OCC] | LC-MS (ESI⁺) m/z 316.1 (M + H)⁺. ¹H NMR (400 MHz, CDCl₃) δ 6.92 − 6.81 (m, 3H), 6.73 (dd, J = 2.0, 10.1 Hz, 1H), 6.10 (d, J = 10.0 Hz, 1H), 4.14 − 4.06 (m, 4H), 3.37 − 3.26 (m, 1H), 2.76 − 2.69 (m, 1H), 2.66 − 2.60 (m, 1H), 2.56 − 2.42 (m, 3H), 2.32 (s, 3H), 2.21 (td, J = 8.4, 13.1 Hz, 1H), 1.45 (t, J = 6.8 Hz, 6H). |
| | 1765-KR | |
| 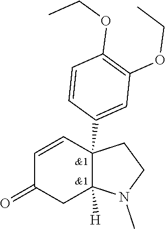 [SMILES: CCOc1ccc([C@@]23C=CC(=O)C[C@@H]2N(C)CC3)cc1OCC] | 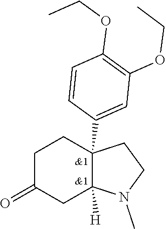 [SMILES: CCOc1ccc([C@@]23CCC(=O)C[C@@H]2N(C)CC3)cc1OCC] | LC-MS (ESI⁺) m/z 318.3 (M + H)⁺. ¹H NMR (400 MHz, CDCl₃) δ 6.95 − 6.89 (m, 2H), 6.88 − 6.83 (m, 1H), 4.10 (m, 4H), 3.18 − 3.09 (m, 1H), 2.94 (t, J = 3.6 Hz, 1H), 2.60 (d, J = 3.6 Hz, 2H), 2.48 − 2.39 (m, 1H), 2.32 (s, 4H), 2.24 − 2.03 (m, 5H), 1.46 (dt, J = 1.6, 7.2 Hz, 6H). |
| 1765-KR | 1763-KR | |
| 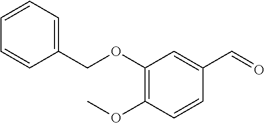 + MeNH₂ | 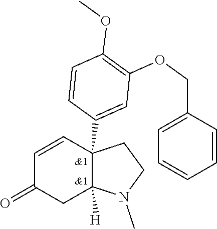 [SMILES: COc1ccc([C@@]23C=CC(=O)C[C@@H]2N(C)CC3)cc1OCc1ccccc1] | LC-MS (ESI⁺) m/z 364.0 (M + H)⁺. ¹H NMR (400 MHz, CDCl₃) δ 7.42 − 7.29 (m, 4H), 6.94 − 6.76 (m, 3H), 6.63 (dd, J = 2.0, 10.1 Hz, 1H), 6.06 (d, J = 10.0 Hz, 1H), 5.24 − 5.12 (m, 2H), 3.94 − 3.86 (m, 3H), 3.26 (dt, J = 2.4, 8.8 Hz, 1H), 2.53 − 2.37 (m, 3H), 2.37 − 2.21 (m, 5H), 2.19 − 2.06 (m, 1H). |
| | 2771-KR | |
| 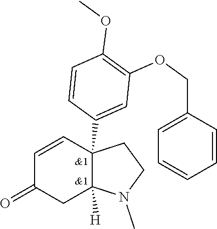 | 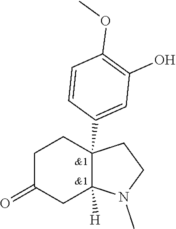 [SMILES: COc1ccc([C@@]23CCC(=O)C[C@@H]2N(C)CC3)cc1O] | LC-MS (ESI⁺) m/z 276.1 (M + H)⁺. ¹H NMR (400 MHz, CDCl₃) δ 8.29 (s, 1H), 6.92 (d, J = 2.4 Hz, 1H), 6.85 − 6.72 (m, 2H), 3.90 (s, 3H), 3.56 − 3.46 (m, 1H), 3.33 (t, J = 4.8 Hz, 1H), 2.90 − 2.68 (m, 2H), 2.60 − 2.50 (m, 4H), 2.44 − 2.33 (m, 2H), 2.28 − 2.05 (m, 4H). |
| 2771-KR | 1793-KR | |
| 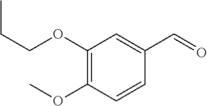 + MeNH₂ | 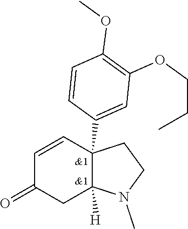 [SMILES: CCCOc1cc([C@@]23C=CC(=O)C[C@@H]2N(C)CC3)ccc1OC] | LC-MS (ESI⁺) m/z 316.3 (M + H)⁺. ¹H NMR (400 MHz, CDCl₃) δ 6.93 − 6.81 (m, 3H), 6.78 − 6.67 (m, 1H), 6.11 (d, J = 10.2 Hz, 1H), 3.98 (t, J = 6.8 Hz, 2H), 3.87 (s, 3H), 3.39 − 3.27 (m, 1H), 2.72 − 2.41 (m, 5H), 2.33 (s, 3H), 2.26 − 2.14 (m, 1H), 1.95 − 1.79 (m, 2H), 1.06 (t, J = 7.4 Hz, 3H). |
| | 709-KR | |
| 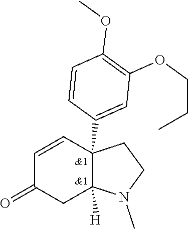 [SMILES: CCCOc1cc([C@@]23C=CC(=O)C[C@@H]2N(C)CC3)ccc1OC] | 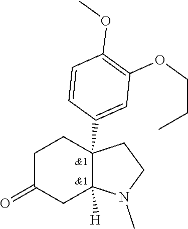 [SMILES: CCCOc1cc([C@@]23CCC(=O)C[C@@H]2N(C)CC3)ccc1OC] | LC-MS (ESI⁺) m/z 318.3 (M + H)⁺. ¹H NMR (400 MHz, CDCl₃) δ 6.97 − 6.88 (m, 2H), 6.88 − 6.82 (m, 1H), 3.99 (t, J = 6.8 Hz, 2H), 3.87 (s, 3H), 3.20 − 3.09 (m, 1H), 2.94 (t, J = 3.4 Hz, 1H), 2.67 − 2.53 (m, 2H), 2.50 − 2.39 (m, 1H), 2.39 − 2.28 (m, 4H), 2.27 − 2.01 (m, 5H), 1.94 − 1.82 (m, 2H), 1.06 (t, J = 7.4 Hz, 3H). |
| 709-KR | 707-KR | |
| 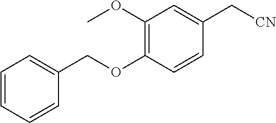 + MeNH₂ | 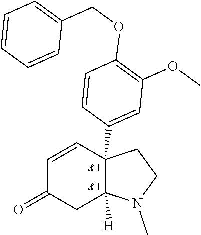 [SMILES: COc1cc([C@@]23C=CC(=O)C[C@@H]2N(C)CC3)ccc1OCc1ccccc1] | LC-MS (ESI⁺) m/z 364.2 (M + H) +; ¹H NMR (400 MHz, CDCl₃) δ 7.49 − 7.43 (m, 2H), 7.38 (d, J = 7.2 Hz, 2H), 7.34 (d, J = 7.2 Hz, 1H), 6.97 − 6.80 (m, 3H), 6.73 (dd, J = 2.0, 10.0 Hz, 1H), 6.11 (d, J = 10.0 Hz, 1H), 5.17 (s, 2H), 3.92 (s, 3H), 3.37 − 3.27 (m, 1H), 2.72 − 2.65 (m, 1H), 2.63 − 2.56 (m, 1H), 2.53 (dd, J = 2.4, 6.4 Hz, 1H), 2.49 (dd, J = 2.4, 4.0 Hz, 1H), 2.44 (dd, J = 2.4, 8.8 Hz, 1H), 2.34 (s, 3H), 2.27 − 2.18 (m, 1H). |
| | 2773-KR | |
| 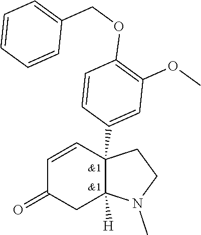 | 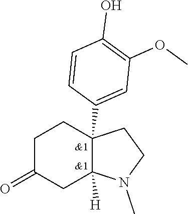 [SMILES: COc1cc([C@@]23CCC(=O)C[C@@H]2N(C)CC3)ccc1O] 1799-KR | LC-MS (ESI⁺) m/z 276.2 (M + H) +; ¹H NMR (400 MHz, CDCl₃) δ 6.96 − 6.84 (m, 3H), 5.60 (s, 1H), 3.93 (s, 3H), 3.21 − 3.09 (m, 1H), 2.96 (t, J = 3.6 Hz, 1H), 2.68 − 2.56 (m, 2H), 2.51 − 2.39 (m, 1H), 2.39 − 2.29 (m, 4H), 2.27 − 2.16 (m, 3H), 2.15 − 2.01 (m, 2H). |
| 2773-KR | | |
| 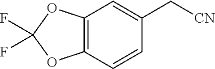 [SMILES: N#CCc1ccc2c(c1)OC(F)(F)O2] + MeNH₂ | 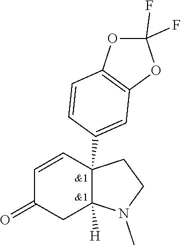 [SMILES: CN1CC[C@]2(c3ccc4c(c3)OC(F)(F)O4)C=CC(=O)C[C@H]12] | LC-MS (ESI⁺) m/z 308.0 (M + H)⁺. ¹H NMR (400 MHz, DMSO-d6) δ = 10.69 − 10.47 (m, 1H), 7.72 (s, 1H), 7.45 (d, J = 8.4 Hz, 1H), 7.28 (dd, J = 1.6, 8.5 Hz, 1H), 7.04 (d, J = 9.2 Hz, 1H), 6.27 (d, J = 10.4 Hz, 1H), 4.06 (s, 1H), 3.57 (dd, J = 7.6, 11.2 Hz, 1H), 3.46 − 3.39 (m, 1H), 2.96 (d, J = 4.4 Hz, 3H), 2.84 − 2.71 (m, 2H), 2.49 − 2.41 (m, 2H). |
| | 2755-KR | |
| 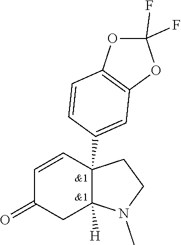 | 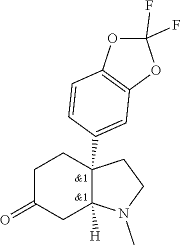 | LC-MS (ESI⁺) m/z 303.1 (M + H)⁺. ¹H NMR (400 MHz, CDCl₃) δ 7.15 − 7.08 (m, 2H), 7.06 − 7.02 (m, 1H), 3.19 (d, J = 2.4 Hz, 1H), 2.96 (s, 1H), 2.61 (d, J = 2.4 Hz, 2H), 2.52 − 2.42 (m, 1H), 2.35 (s, 3H), 2.30 − 2.22 (m, 1H), 2.20 − 2.09 (m, 4H), 1.71 − 1.41 (m, 1H). |
| 2755-KR | 2753-KR | |

Procedures similar to those described above in Example 1 could also be used to prepare ketones in the following table from the appropriate starting materials:

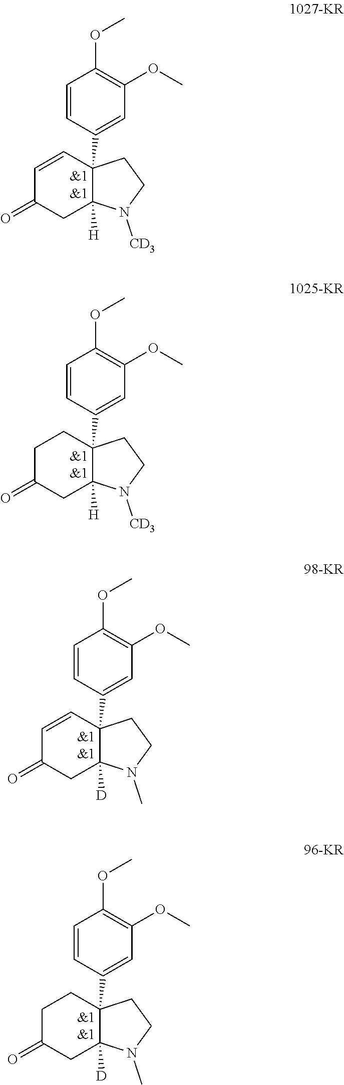 [SMILES: [2H]C([2H])([2H])N1CC[C@]2(c3ccc(OC)c(OC)c3)C=CC(=O)C[C@H]12 ; [2H]C([2H])([2H])N1CC[C@]2(c3ccc(OC)c(OC)c3)CCC(=O)C[C@H]12 ; [2H][C@]12CC(=O)C=C[C@@]1(c1ccc(OC)c(OC)c1)CCN2C ; [2H][C@]12CC(=O)CC[C@@]1(c1ccc(OC)c(OC)c1)CCN2C]

### Example 2: SFC Separation of rac-3a-(3,4-dimethoxyphenyl)-1-methyloctahydro-6H-indol-6-one to give (−)-Mesembrine (0001) and (+)-Mesembrine (0002)

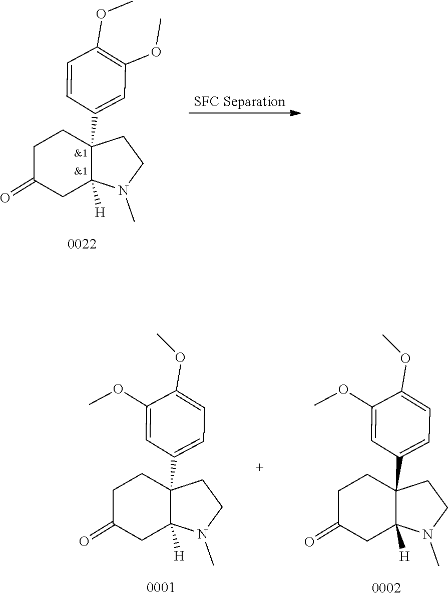 [SMILES: COc1ccc([C@@]23CCC(=O)C[C@@H]2N(C)CC3)cc1OC ; COc1ccc([C@@]23CCC(=O)C[C@@H]2N(C)CC3)cc1OC ; COc1ccc([C@]23CCC(=O)C[C@H]2N(C)CC3)cc1OC]

Compound 0022 (15 g, 90% purity) was subjected to separation by SFC (column: DAICEL CHIRALCEL OD (250 mm*50 mm, 10 um); mobile phase: [Neu-MeOH]; B %: 25%-25%, 2; 1230 min) to give 0001 (peak 1, 5.4 g, free base, 36%) as yellow oil and 0002 (peak 2, 5.6 g, free base, 37%) as yellow oil.

0001: LC-MS (ESI⁺) m/z 290.4 (M+H)^{+ 1}H NMR (400 MHz, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

0002: LC-MS (ESI⁺) m/z 290.4 (M+H)^{+ 1}H NMR (400 MHz, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

A procedure similar to that described above in Example 2 were used to separate the enantiomers of certain ketones as shown in the following table:

| Ketone and Separation Conditions | Products | Characterization |
|---|---|---|
| 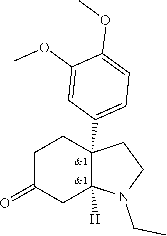 [SMILES: CCN1CC[C@]2(c3ccc(OC)c(OC)c3)CCC(=O)C[C@H]12] 1001-KR | Peak 1: 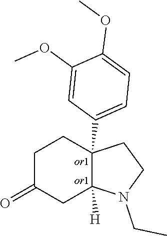 [SMILES: CCN1CC[C@]2(c3ccc(OC)c(OC)c3)CCC(=O)C[C@H]12] | LC-MS (ESI⁺) m/z 302.2 (M + H)⁺; ¹H NMR (400 MHz, MeOD) δ 7.02 − 6.97 (m, 2H), 6.94 (s, 1H), 3.83 (d, J = 9.2 Hz, 6H), 3.17 (s, 2H), 2.96 − 2.85 (m, 1H), 2.76 − 2.64 (m, 1H), 2.63 − 2.51 (m, 1H), 2.43 − 2.31 (m, 1H), 2.30 − 2.21 (m, 3H), 2.20 − 2.04 (m, 4H), 1.10 (t, J = 7.2 Hz, 3H). |
| Column: Chiralpak AS-3 50 × 4.6 mm | 1001-K | |
| I.D., 3 um, Mobile phase: Phase A for CO₂, and Phase B for IPA (0.05% DEA); Gradient elution: B in A from 5% to 40%, Flow rate: 3 mL/min; Detector: PDA; Column Temp: 35 C.; Back Pressure: 100 Bar | Peak 2: 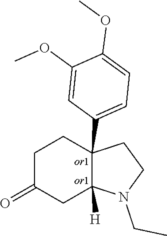 [SMILES: CCN1CC[C@@]2(c3ccc(OC)c(OC)c3)CCC(=O)C[C@@H]12] | LC-MS (ESI⁺) m/z 302.2 (M + H)⁺; ¹H NMR (400 MHz, CDCl₃) δ 7.05 − 6.78 (m, 3H), 3.89 (d, J = 7.6 Hz, 6H), 3.25 (t, J = 6.8 Hz, 1H), 3.15 (s, 1H), 3.00 − 2.84 (m, 1H), 2.71 − 2.55 (m, 2H), 2.52 − 2.38 (m, 1H), 2.35 − 2.23 (m, 2H), 2.23 − 2.00 (m, 5H), 1.10 (t, J = 7.2 Hz, 3H) |
| | 1001-L | |
| 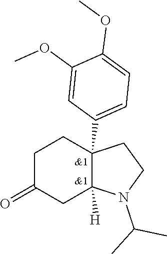 [SMILES: COc1ccc([C@@]23CCC(=O)C[C@@H]2N(C(C)C)CC3)cc1OC] | Peak 1: 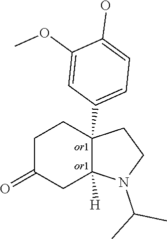 | LC-MS (ESI⁺) m/z 318.3 (M + H) ⁺; ¹H NMR (400 MHz, CDCl₃) δ 6.99 − 6.77 (m, 3H), 3.90 (d, J = 6.4 Hz, 6H), 3.65 − 3.41 (m, 1H), 3.24 − 3.07 (m, 1H), 3.06 − 2.84 (m, 1H), 2.81 − 2.49 (m, 3H), 2.41 (dd, J = 6.0, 11.6 Hz, 2H), 2.27 − 1.91 (m, 4H), 1.25 − 1.06 (m, 3H), 1.05 − 0.77 (m, 3H). |
| 1007-KR | | |
| | Peak 2: | LC-MS (ESI⁺) m/z 318.3 (M + H) ⁺; ¹H |
| column: DAICEL CHIRALPAK AS (250 mm*30 mm, 10 um); mobile phase: [IPA-ACN]; B%: 20%-20%, A10; 470 min | 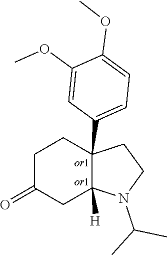 [SMILES: COc1ccc([C@]23CCC(=O)C[C@H]2N(C(C)C)CC3)cc1OC] | NMR (400 MHz, CDCl₃) δ 6.84 (s, 2H), 6.79 − 6.75 (m, 1H), 3.82 (d, J = 6.4 Hz, 6H), 3.54 − 3.37 (m, 1H), 3.06 (s, 1H), 2.98 − 2.80 (m, 1H), 2.70 − 2.43 (m, 3H), 2.41 − 2.23 (m, 2H), 2.19 − 1.90 (m, 4H), 1.17 − 1.00 (m, 3H), 0.89 (s, 3H). |
| | 1007-L | |
| 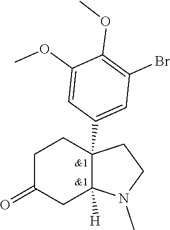 [SMILES: COc1cc([C@@]23CCC(=O)C[C@@H]2N(C)CC3)cc(Br)c1OC] 825-KR | Peak 1: 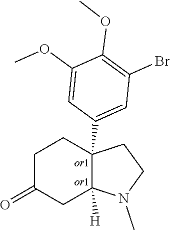 [SMILES: COc1cc([C@@]23CCC(=O)C[C@@H]2N(C)CC3)cc(Br)c1OC] | LC-MS (ESI⁺) m/z 368.0 (M + H)⁺ ¹H NMR (400 MHz, CDCl₃) δ 7.07 (d, J = 2.0 Hz, 1H), 6.78 (d, J = 2.0 Hz, 1H), 3.80 (d, J = 11.3 Hz, 6H), 3.18 − 3.01 (m, 1H), 2.85 (s, 1H), 2.53 ( s, 2H), 2.46 − 2.33 (m, 1H), 2.30 − 2.21 (m, 4H), 2.20 − 2.15 (m, 1H), 2.15 − 2.07 (m, 2H), 2.06 − 1.97 (m, 2H), 1.97 − 1.97 (m, 1H), 1.87 − 1.33 (m, 2H). |
| | 825-K | |
| | Peak 2: 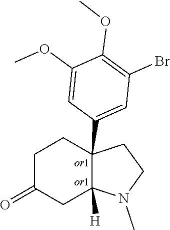 [SMILES: COc1cc([C@]23CCC(=O)C[C@H]2N(C)CC3)cc(Br)c1OC] | LC-MS (ESI⁺) m/z 368.0 (M + H)⁺ ¹H NMR (400 MHz, CDCl₃) δ 7.07 (d, J = 2.4 Hz, 1H), 6.78 (d, J = 2.0 Hz, 1H), 3.80 (d, J = 11.2 Hz, 6H), 3.15 − 3.02 (m, 1H), 2.85 (t, J = 3.2 Hz, 1H), 2.53 (d, J = 3.6 Hz, 2H), 2.45 − 2.32 (m, 1H), 2.30 − 2.21 (m, 4H), 2.16 (s, 1H), 2.15 − 2.07 (m, 2H), 2.06 − 1.96 (m, 2H). |
| | 825-L | |
| 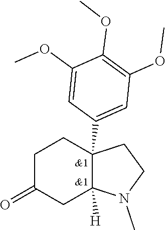 [SMILES: COc1cc([C@@]23CCC(=O)C[C@@H]2N(C)CC3)cc(OC)c1OC] 801-KR | Peak 1: 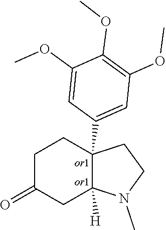 [SMILES: COc1cc([C@@]23CCC(=O)C[C@@H]2N(C)CC3)cc(OC)c1OC] | LC-MS (ESI+) m/z 320.3 (M + H)⁺. ¹H NMR (400 MHz, CDCl₃) δ 6.59 (s, 2H), 3.96 − 3.80 (m, 9H), 3.16 (t, J = 6.8 Hz, 1H), 2.96 (s, 1H), 2.63 (s, 2H), 2.34 (s, 3H), 2.24 (s, 1H), 2.23 − 2.19 (m, 2H), 2.18 − 2.09 (m, 2H), 1.57 (s, 2H). |
| column: Daicel ChiralPak IG | 801-K | |
| (250*30 mm, 10 um); mobile phase: | | |
| [Neu-MeOH]; B%: 25%- 25%, C14.55; 146 min | Peak 2: 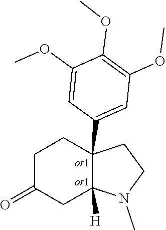 [SMILES: COc1cc([C@]23CCC(=O)C[C@H]2N(C)CC3)cc(OC)c1OC] | LC-MS (ESI+) m/z 320.1 (M + H)⁺. ¹H NMR (400 MHz, CDCl₃) 6.59 (s, 2H), 3.96 − 3.80 (m, 9H), 3.20 − 3.10 (m, 1H), 2.95 (Br s, 1H), 2.62 (Br d, J = 3.2 Hz, 2H), 2.33 (s, 3H), 2.25 (s, 1H), 2.23 − 2.19 (m, 2H), 2.18 − 2.10 (m, 2H), 1.58 (s, 2H). |
| | 801-L | |
| 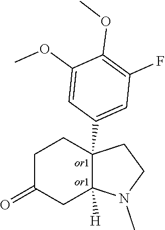 [SMILES: COc1cc([C@@]23CCC(=O)C[C@@H]2N(C)CC3)cc(F)c1OC] 813-K | Peak 1: 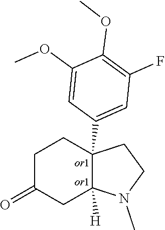 [SMILES: COc1cc([C@@]23CCC(=O)C[C@@H]2N(C)CC3)cc(F)c1OC] | LC-MS (ESI⁺) m/z 308.2 (M + H)⁺ ¹H NMR (400 MHz, CDCl₃) δ = 6.77 (br d, J = 12.8 Hz, 1H), 6.70 (s, 1H), 3.92 (br d, J = 13.6 Hz, 6H), 3.14 (t, J = 6.4 Hz, 1H), 2.90 (s, 1H), 2.70 − 2.53 (m, 2H), 2.52 − 2.40 (m, 1H), 2.38 − 2.27 (m, 4H), 2.26 − 2.13 (m, 3H), 2.13 − 2.06 (m, 2H) |
| column: Daicel ChiralPak IG | 813-K | |
| (250*30 mm, 10 um); mobile phase: | | |
| [0.1% NH₃H2O MeOH]; B%: 20%- 20%, C12.05; 109 min | Peak 2: 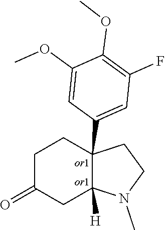 [SMILES: COc1cc([C@]23CCC(=O)C[C@H]2N(C)CC3)cc(F)c1OC] | LC-MS (ESI⁺) m/z 308.2 (M + H)⁺ ¹H NMR (400 MHz, CDCl₃) δ 6.77 (br d, J = 12.8 Hz, 1H), 6.70 (s, 1H), 3.92 (br d, J = 13.6 Hz, 6H), 3.14 (t, J = 6.4 Hz, 1H), 2.90 (s, 1H), 2.70 − 2.53 (m, 2H), 2.52 − 2.40 (m, 1H), 2.38 − 2.27 (m, 4H), 2.26 − 2.13 (m, 3H), 2.13 − 2.06 (m, 2H). |
| | 813-L | |
| 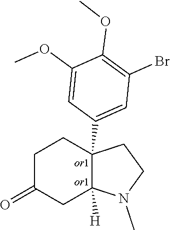 [SMILES: COc1cc([C@@]23CCC(=O)C[C@@H]2N(C)CC3)cc(Br)c1OC] 825-K | Peak 1: 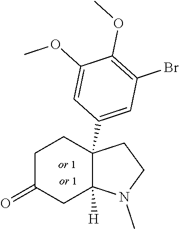 [SMILES: COc1cc([C@@]23CCC(=O)C[C@@H]2N(C)CC3)cc(Br)c1OC] | LC-MS (ESI⁺) m/z 368.0 (M + H)⁺ ¹H NMR (400 MHz, CDCl₃) δ 7.07 (d, J = 2.0 Hz, 1H), 6.78 (d, J = 2.0 Hz, 1H), 3.80 (d, J = 11.3 Hz, 6H), 3.18 − 3.01 (m, 1H), 2.85 (s, 1H), 2.53 ( s, 2H), 2.46 − 2.33 (m, 1H), 2.30 − 2.21 (m, 4H), 2.20 − 2.15 (m, 1H), 2.15 − 2.07 (m, 2H), 2.06 − 1.97 (m, 2H), 1.97 − 1.97 (m, 1H), 1.87 − 1.33 (m, 2H). |
| Column: Diacel Chiralpak IE | 825-K | |
| (250 mm*30 mm, 10 um); mobile | | |
| phase: [Neu-ETOH]; B%: 20%- | Peak 2: | LC-MS (ESI*) m/z 368.0 (M + H)⁺ ¹H |
| 20%, C20; 80 min | | NMR (400 MHz, CDCl₃) δ 7.07 (d, J = |
| | 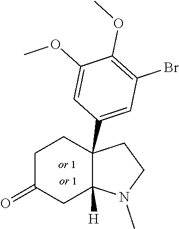 [SMILES: COc1cc([C@]23CCC(=O)C[C@H]2N(C)CC3)cc(Br)c1OC] | 2.4 Hz, 1H), 6.78 (d, J = 2.0 Hz, 1H), 3.80 (d, J = 11.2 Hz, 6H), 3.15 − 3.02 (m, 1H), 2.85 (t, J = 3.2 Hz, 1H), 2.53 (d, J = 3.6 Hz, 2H), 2.45 − 2.32 (m, 1H), 2.30 − 2.21 (m, 4H), 2.16 (s, 1H), 2.15 − 2.07 (m, 2H), 2.06 − 1.96 (m, 2H). |
| | 825-L | |
| 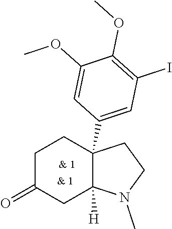 [SMILES: COc1cc([C@@]23CCC(=O)C[C@@H]2N(C)CC3)cc(I)c1OC] 831-KR | Peak 1: 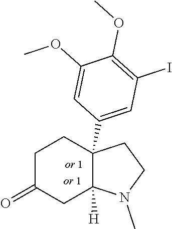 [SMILES: COc1cc([C@@]23CCC(=O)C[C@@H]2N(C)CC3)cc(I)c1OC] | LC-MS (ESI⁺) m/z 416.0 (M + H) ⁺; ¹H NMR (400 MHz, CDCl₃) δ 7.26 (d, J = 2.0 Hz, 1H), 6.80 (d, J = 2.0 Hz, 1H), 3.78 (d, J = 16.0 Hz, 6H), 3.10 (t, J = 6.8 Hz, 1H), 2.88 (s, 1H), 2.62 − 2.48 (m, 2H), 2.45 − 2.33 (m, 1H), 2.32 − 2.23 (m, 4H), 2.21 − 2.00 (m, 5H). |
| Column: DAICEL CHIRALCEL | 831-K | |
| OD (250 mm*30 mm, 10 um); | | |
| mobile phase: [Neu-MeOH]; B%: | Peak 2: | LC-MS (ESI⁺) m/z 416.0 (M + H)⁺; ¹H |
| 25%-25%, A2.2; 22 min | | NMR (400 MHz, CDCl₃) δ 7.35 (d, J = |
| | 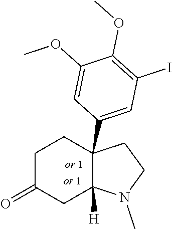 [SMILES: COc1cc([C@]23CCC(=O)C[C@H]2N(C)CC3)cc(I)c1OC] | 2.0 Hz, 1H), 6.89 (d, J = 2.0 Hz, 1H), 3.88 (d, J = 16.0 Hz, 6H), 3.25 − 3.14 (m, 1H), 2.99 (s, 1H), 2.73 − 2.58 (m, 2H), 2.54 − 2.43 (m, 1H), 2.38 (s, 4H), 2.30 − 2.10 (m, 5H). |
| | 831-L | |
| 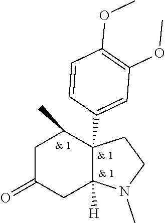 [SMILES: COc1ccc([C@]23CCN(C)[C@H]2CC(=O)C[C@H]3C)cc1OC] 901-KR | Peak 1: 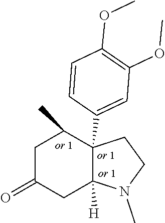 [SMILES: COc1ccc([C@]23CCN(C)[C@H]2CC(=O)C[C@H]3C)cc1OC] | LC-MS (ESI⁺) m/z 304.3 (M + H) ⁺ ¹H NMR (400 MHz, CDCl₃) δ = 7.00 − 6.81 (m, 3H), 3.90 (d, J = 6.5 Hz, 6H), 3.07 (s, 1H), 2.83 − 2.61 (m, 3H), 2.52 − 2.40 (m, 2H), 2.36 − 2.18 (m, 6H), 2.02 (d, J = 6.8 Hz, 1H), 0.78 (d, J = 6.1 Hz, 3H). |
| Column: Chiralpak AD-3 50 × 4.6 mm | 901-K | |
| I.D., 3 um Mobile phase: Phase A for | | |
| CO₂, and Phase B for MeOH (0.05% | Peak 2: | LC-MS (ESI⁺) m/z 304.3 (M + H)⁺. ¹H |
| DEA); Gradient elution: MeOH | | NMR (400 MHz, CDCl₃) δ = 7.00 − |
| (0.05% DEA) in CO₂ from 5% to 40%, Flow rate: 3 mL/min; Detector: PDA; Column Temp: 35 C.; Back Pressure: 100 Bar | 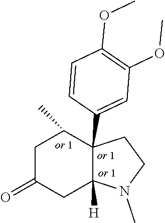 [SMILES: COc1ccc([C@@]23CCN(C)[C@@H]2CC(=O)C[C@@H]3C)cc1OC] | 6.81 (m, 3H), 3.90 (d, J = 6.5 Hz, 6H), 3.07 (s, 1H), 2.83 − 2.61 (m, 3H), 2.52 − 2.40 (m, 2H), 2.36 − 2.18 (m, 6H), 2.02 (br d, J = 6.8 Hz, 1H), 0.78 (d, J = 6.1 Hz, 3H). |
| | 901-L | |
| 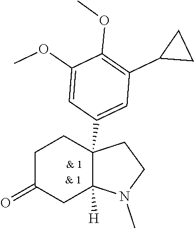 [SMILES: COc1cc([C@@]23CCC(=O)C[C@@H]2N(C)CC3)cc(C2CC2)c1OC] 819-KR | Peak 1: 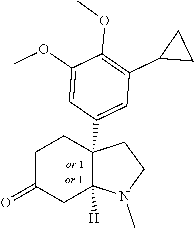 [SMILES: COc1cc([C@@]23CCC(=O)C[C@@H]2N(C)CC3)cc(C2CC2)c1OC] | LC-MS (ESI⁺) m/z 330.2 (M + H)⁺. ¹H NMR (400 MHz, CDCl₃) δ 6.74 (d, J = 2.0 Hz, 1H), 6.41 (d, J = 1.6 Hz, 1H), 3.89 (d, J = 5.4 Hz, 6H), 3.22 − 3.09 (m, 1H), 2.94 (br s, 1H), 2.62 (br s, 2H), 2.51 − 2.40 (m, 1H), 2.34 (s, 4H), 2.29 − 2.05 (m, 6H), 1.08 − 0.95 (m, 2H), 0.74 − 0.62 (m, 2H). |
| column: DAICEL CHIRALCEL | 819-K | |
| OD(250 mm*30 mm, 10 um);mobile | | |
| phase: [Neu-IPA]; B%: 30%- | Peak 2: | ¹H NMR (400 MHz, CDCl₃) δ = 6.65 (d, |
| 30%, C7.85; 71 min | | J = 2.4 Hz, 1H), 6.32 (d, J = 2.0 Hz, |
| | 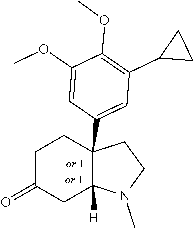 [SMILES: COc1cc([C@]23CCC(=O)C[C@H]2N(C)CC3)cc(C2CC2)c1OC] | 1H), 3.80 (d, J = 5.2 Hz, 6H), 3.06 (br t, J = 7.2 Hz, 1H), 2.84 (br s, 1H), 2.52 (br d, J = 3.6 Hz, 2H), 2.42 − 2.30 (m, 1H), 2.29 − 2.22 (m, 4H), 2.21 − 1.96 (m, 6H), 0.95 − 0.88 (m, 2H), 0.62 − 0.55 (m, 2H). |
| | 819-L | |
| 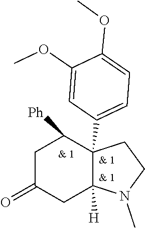 [SMILES: COc1ccc([C@]23CCN(C)[C@H]2CC(=O)C[C@H]3c2ccccc2)cc1OC] 947-KR | Peak 1: 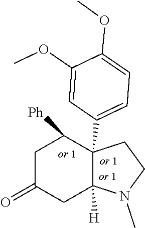 [SMILES: COc1ccc([C@]23CCN(C)[C@H]2CC(=O)C[C@H]3c2ccccc2)cc1OC] | LC-MS (ESI⁺) m/z 366.4 (M + H) ⁺ ¹H NMR (CDCl₃): δ 7.00-7.14 (3H, m), 6.41-6.76 (5H, m), 3.83 (3H, s), 3.65 (3H, s), 3.32-3.46 (¹H, m), 2.83-3.13 (4H, m), 2.72-2.81 (¹H, m), 2.34-2.48 (¹H, m), 2.21 (3H, s), 2.02-2.18 (2H, m), 1.94 (¹H, s), 1.79-1.87 (¹H, m). |
| column: Phenomenex-Cellulose-2 | 947-K | |
| (250 mm*30 mm, 10 um); mobile | | |
| phase: [0.1% NH₃H₂O MeOH]; B%: | Peak 2: | LC-MS (ESI⁺) m/z 366.4 (M + H) ⁺ ¹H |
| 50%-50%, 2.5; 25 min | | NMR (CDCl₃): δ 7.10-7.23 (3H, m), |
| | 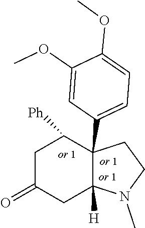 [SMILES: COc1ccc([C@@]23CCN(C)[C@@H]2CC(=O)C[C@@H]3c2ccccc2)cc1OC] | 6.43-6.88 (5H, m), 3.92 (3H, s), 3.75 (3H, s), 3.40-3.53 (¹H, m), 3.01-3.36 (4H, m), 2.94 (¹H, s), 2.48-2.58 (1H, m), 2.36 (3H, s), 2.17-2.34 (2H, m), 1.99 (¹H, d, J-3.2 Hz). |
| | 947-L | |
| 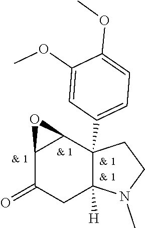 [SMILES: COc1ccc([C@]23CCN(C)[C@H]2CC(=O)[C@H]2O[C@H]23)cc1OC] 1931-KR | Peak 1: 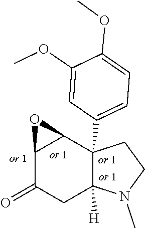 [SMILES: COc1ccc([C@]23CCN(C)[C@H]2CC(=O)[C@H]2O[C@H]23)cc1OC] | LC-MS (ESI*) m/z 304.1 (M + H) ⁺; ¹H NMR (400 MHz, CDCl₃) δ 6.88 − 6.80 (m, 2H), 6.73 (s, 1H), 3.89 (s, 6H), 3.58 (d, J = 4.0 Hz, 1H), 3.48 (d, J = 4.0 Hz, 1H), 3.37 − 3.25 (m, 1H), 2.96 (br t, J = 12.4 Hz, 2H), 2.78 − 2.65 (m, 1H), 2.63 − 2.49 (m, 3H), 2.35 (br s, 3H). |
| column: DAICEL CHIRALPAK | 1931-K | LC-MS (ESI⁺) m/z 304.1 (M + H) ⁺; ¹H |
| IE(250 mm*30 mm, 10 um);mobile | | NMR (400 MHz, CDCl₃) δ 6.89 − 6.80 |
| phase: [Neu-ETOH]; B%: 20%- | Peak 2: | (m, 2H), 6.73 (br s, 1H), 3.97 − 3.83 (m, |
| 20%, C20; 80 min | | 6H), 3.59 (br d, J = 3.0 Hz, 1H), 3.48 |
| | 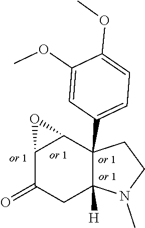 [SMILES: COc1ccc([C@@]23CCN(C)[C@@H]2CC(=O)[C@@H]2O[C@@H]23)cc1OC] | (d, J = 4.0 Hz, 1H), 3.41 − 3.27 (m, 1H), 3.07 − 2.82 (m, 2H), 2.79 − 2.65 (m, 1H), 2.63 − 2.51 (m, 3H), 2.36 (br s, 2H). |
| | 1931-L | |
| 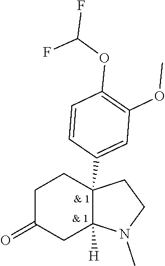 [SMILES: COc1cc([C@@]23CCC(=O)C[C@@H]2N(C)CC3)ccc1OC(F)F] 2723-KR | Peak 1: 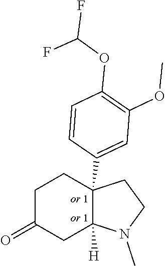 [SMILES: COc1cc([C@@]23CCC(=O)C[C@@H]2N(C)CC3)ccc1OC(F)F] | LC-MS (ESI⁺) m/z 326.2 (M + H)⁺ ¹H NMR (400 MHz, CDCl₃) δ 7.15 (d, J = 8.0 Hz, 1H), 7.02 − 6.88 (m, 2H), 6.76 − 6.32 (m, 1H), 3.91 (s, 3H), 3.24 − 3.08 (m, 1H), 3.03 − 2.88 (m, 1H), 2.70 − 2.55 (m, 2H), 2.49 − 2.42 (m, 1H), 2.34 (s, 3H), 2.27 − 2.19 (m, 2H), 2.19 − 2.13 (m, 2H), 1.32 − 1.25 (m, 2H). |
| column: Daicel ChiralPak IG | 2723-K | |
| (250*30 mm, 10 um); mobile phase: | | |
| [0.1% NH₃H₂O MeOH]; B%: 25%- | Peak 2: | LC-MS (ESI⁺) m/z 326.2 (M + H)⁺ ¹H |
| 25%, A3.6; 45 min | | NMR (400 MHz, CDCl₃) δ 7.15 (d, J = |
| | 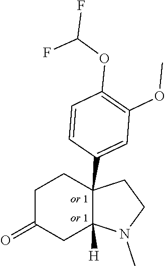 [SMILES: COc1cc([C@]23CCC(=O)C[C@H]2N(C)CC3)ccc1OC(F)F] | 8.4 Hz, 1H), 7.03 − 6.86 (m, 2H), 6.56 (t, J = 75.2 Hz, 1H), 3.91 (s, 3H), 3.34 − 3.14 (m, 1H), 3.12 − 2.92 (m, 1H), 2.68 − 2.62 (m, 1H), 2.53 − 2.42 (m, 2H), 2.40 (br s, 3H), 2.25 − 2.12 (m, 4H), 1.37 − 1.31 (m, 1H), 1.26 (s, 2H). |
| | 2723-L | |
| 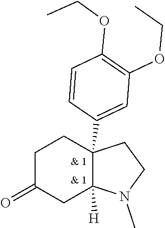 [SMILES: CCOc1ccc([C@@]23CCC(=O)C[C@@H]2N(C)CC3)cc1OCC] 1763-KR | Peak 1: 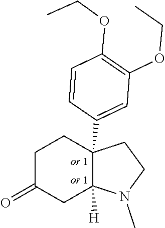 [SMILES: CCOc1ccc([C@@]23CCC(=O)C[C@@H]2N(C)CC3)cc1OCC] | LC-MS (ESI⁺) m/z 317.9 (M + H)⁺. ¹H NMR (400 MHz, CDCl₃) δ 6.94 − 6.82 (m, 3H), 4.10 (quin, J = 7.2 Hz, 4H), 3.18 − 3.09 (m, 1H), 2.94 (t, J = 3.2 Hz, 1H), 2.60 (d, J = 3.6 Hz, 2H), 2.48 − 2.39 (m, 1H), 2.37 − 2.29 (m, 4H), 2.24 − 2.04 (m, 5H), 1.45 (dt, J = 1.2, 7.2 Hz, 6H). |
| condition: column: DAICEL | 1763-K | |
| CHIRALPAK AS | | |
| (250 mm*30 mm, 10 um); mobile phase: [0.1% NH₃H₂O MeOH]; B%: 40%-40%, A3; 30 min | Peak 2: 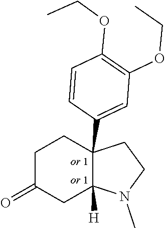 [SMILES: CCOc1ccc([C@]23CCC(=O)C[C@H]2N(C)CC3)cc1OCC] | LC-MS (ESI⁺) m/z 317.9 (M + H)⁺. ¹H NMR (400 MHz, CDCl₃) δ 6.95 − 6.82 (m, 3H), 4.10 (quin, J = 7.2 Hz, 4H), 3.18 − 3.10 (m, 1H), 2.95 (t, J = 3.2 Hz, 1H), 2.61 (d, J = 3.6 Hz, 2H), 2.48 − 2.40 (m, 1H), 2.37 − 2.30 (m, 4H), 2.24 − 2.05 (m, 5H), 1.46 (dt, J = 1.2, 7.2 Hz, 6H). |
| | 1763-L | |
| 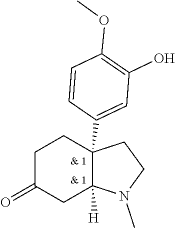 [SMILES: COc1ccc([C@@]23CCC(=O)C[C@@H]2N(C)CC3)cc1O] 1793-KR | Peak 1: 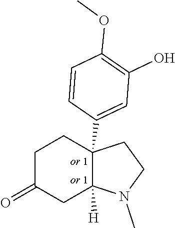 [SMILES: COc1ccc([C@@]23CCC(=O)C[C@@H]2N(C)CC3)cc1O] | LC-MS (ESI*) m/z 276.2 (M + H)⁺. ¹H NMR (400 MHz, CDCl₃) δ 6.98 (s, 1H), 6.83 (s, 2H), 3.90 (s, 3H), 3.26 − 3.21 (m, 1H), 3.05 (t, J = 4.0 Hz, 1H), 2.65 (t, J = 3.2 Hz, 2H), 2.47 − 2.37 (m, 5H), 2.25 (d, J = 10.4 Hz, 1H), 2.21 − 2.09 (m, 4H). |
| First separation: column: DAICEL | 1793-K | |
| CHIRALPAK IG (250 mm *30 mm, | | |
| 10 um); mobile phase: | Peak 2: | LC-MS (ESI⁺) m/z 276.2 (M + H)⁺. ¹H |
| [0.1% NH₃H₂O MeOH]; B%: 60%- | | NMR (400 MHz, CDCl₃) δ 6.97 (s, |
| 60%, A3; 30 min). Second separation column: DAICEL CHIRALPAK IG (250 mm* 30 mm, 10 um); mobile phase: [ACN/MeOH (0.1% NH₃ H₂O)]; B%: 60%-60%, A3; 25min) | 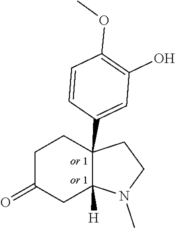 [SMILES: COc1ccc([C@]23CCC(=O)C[C@H]2N(C)CC3)cc1O] | 1H), 6.83 (s, 2H), 3.90 (s, 3H), 3.25 (t, J = 7.2 Hz, 1H), 3.06 (t, J = 4.0 Hz, 1H), 2.66 (t, J = 4.0 Hz, 2H), 2.46 − 2.35 (m, 5H), 2.26 (d, J = 8.9 Hz, 1H), 2.21 − 2.09 (m, 4H). |
| | 1793-L | |
| 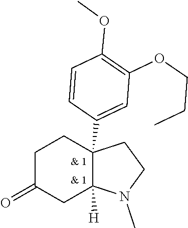 [SMILES: CCCOc1cc([C@@]23CCC(=O)C[C@@H]2N(C)CC3)ccc1OC] 707-KR | Peak 1: 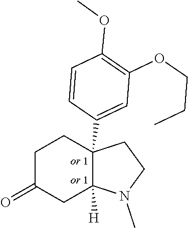 [SMILES: CCCOc1cc([C@@]23CCC(=O)C[C@@H]2N(C)CC3)ccc1OC] | LC-MS (ESI⁺) m/z 318.3 (M + H)⁺. ¹H NMR (400 MHz, CDCl₃) δ 6.97 − 6.89 (m, 2H), 6.88 − 6.82 (m, 1H), 3.99 (t, J = 6.8 Hz, 2H), 3.87 (s, 3H), 3.22 − 3.08 (m, 1H), 2.95 (t, J = 3.6 Hz, 1H), 2.61 (d, J = 3.2 Hz, 2H), 2.51 − 2.39 (m, 1H), 2.38 − 2.29 (m, 4H), 2.28 − 2.16 (m, 3H), 2.15 − 2.02 (m, 2H), 1.93 − 1.83 (m, 2H), 1.06 (t, J = 7.4 Hz, 3H). |
| column: DAICEL CHIRALPAK IG | 707-K | |
| (250 mm*30 mm, 10 um); mobile | | |
| phase: [0.1% NH₃ H₂O MeOH]; B%: | Peak 2: | LC-MS (ESI⁺) m/z 318.3 (M + H)⁺. |
| 25%-25%, C6.8; 88 min | | ¹H NMR (400 MHz, CDCl₃) δ 6.98 − |
| | 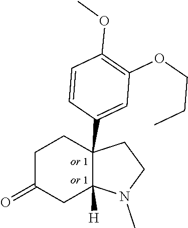 [SMILES: CCCOc1cc([C@]23CCC(=O)C[C@H]2N(C)CC3)ccc1OC] | 6.89 (m, 2H), 6.88 − 6.82 (m, 1H), 3.99 (t, J = 6.8 Hz, 2H), 3.87 (s, 3H), 3.19 − 3.10 (m, 1H), 2.94 (t, J = 3.4 Hz, 1H), 2.66 − 2.55 (m, 2H), 2.50 − 2.39 (m, 1H), 2.38 − 2.28 (m, 4H), 2.28 − 2.16 (m, 3H), 2.15 − 2.02 (m, 2H), 1.95 − 1.82 (m, 2H), 1.06 (t, J = 7.4 Hz, 3H). |
| | 707-L | |
| 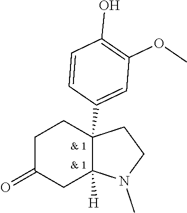 [SMILES: COc1cc([C@@]23CCC(=O)C[C@@H]2N(C)CC3)ccc1O] 1799-KR | Peak 1: 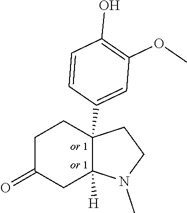 [SMILES: COc1cc([C@@]23CCC(=O)C[C@@H]2N(C)CC3)ccc1O] | LC-MS (ESI⁺) m/z 276.2 (M + H) ⁺; ¹H NMR (400 MHz, CDCl₃) δ 7.04 − 6.78 (m, 3H), 5.56 (s, 1H), 3.94 (s, 3H), 3.17 (s, 1H), 2.98 (s, 1H), 2.63 (s, 2H), 2.50 − 2.41 (m, 1H), 2.35 (s, 4H), 2.29 − 2.00 (m, 5H). |
| | 1799-K | |
| | Peak 2: | LC-MS (ESI⁺) m/z 276.2 (M + H) ⁺; ¹H |
| column: DAICEL CHIRALPAK IC | | NMR (400 MHz, CDCl₃) δ 6.91 (s, |
| (250 mm* 30 mm, 10 um); mobile phase: [0.1% NH₃ H₂O MeOH]; B%: 30%-30%, A2.5; 30 min | 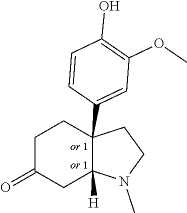 [SMILES: COc1cc([C@]23CCC(=O)C[C@H]2N(C)CC3)ccc1O] | 3H), 5.56 (s, 1H), 3.94 (s, 3H), 3.27 − 3.10 (m, 1H), 3.07 − 2.89 (m, 1H), 2.72 − 2.56 (m, 2H), 2.49 − 2.31 (m, 5H), 2.27 − 1.95 (m, 5H). |
| | 1799-L | |

A procedure similar to that described above in Example 2 could also be used to isolate the ketones shown in the following table:

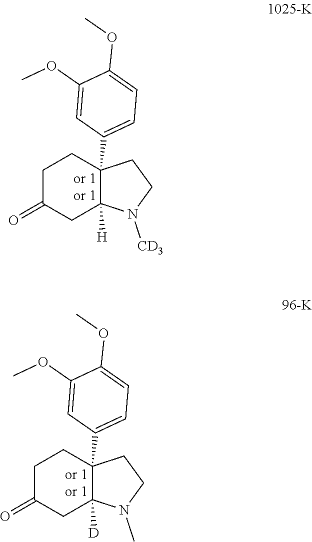 [SMILES: [2H]C([2H])([2H])N1CC[C@]2(c3ccc(OC)c(OC)c3)CCC(=O)C[C@H]12 ; [2H][C@]12CC(=O)CC[C@@]1(c1ccc(OC)c(OC)c1)CCN2C]

### Example 3: Synthesis of (3aS,6R,7aS)-3a-(3,4-dimethoxyphenyl)-1-methyloctahydro-1H-indol-6-ol (0018)

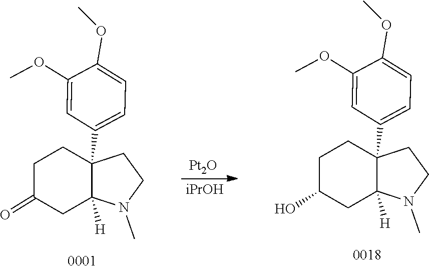

A mixture of 0001 (200 mg, 691 umol) and PtO₂ (20.0 mg, 88.0 umol) in TPA (4 mL) was degassed and purged with N₂ 3 times. The mixture was allowed to stir at 25° C. for 16 hr under an atmosphere of N₂. The reaction mixture was filtered and concentrated under reduced pressure. The residue was purified by re-crystallization from EtOH (1 mL) at 25° C. to give 0018 (100 mg, 49%) as a white solid. LC-MS (ESI⁺) m/z 292.4 (M+H). ¹H NMR (400 MHz, CDCl₃) δ 6.86-6.78 (m, 2H), 6.77-6.71 (m, 1H), 3.86 (s, 1H), 3.81 (d, J=6.8 Hz, 6H), 3.30 (dt, J=6.8, 9.6 Hz, 1H), 2.83 (s, 1H), 2.40 (s, 3H), 2.33-2.20 (m, 1H), 2.09 (dd, J=2.8, 14.8 Hz, 1H), 1.90-1.82 (m, 2H), 1.78 (dd, J=6.8, 11.6 Hz, 1H), 1.67-1.62 (m, 2H), 1.57 (td, J=2.8, 14.8 Hz, 1H), 1.39-1.30 (m, 2H).

Compounds in the table below were prepared from the appropriate ketone starting material according to a procedure similar to that described above in Example 3:

| ID | Structure | Characterization |
|---|---|---|
| 1763 | 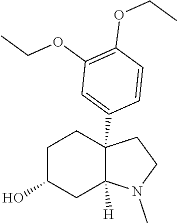 [SMILES: CCOc1ccc([C@@]23CC[C@@H](O)C[C@@H]2N(C)CC3)cc1OCC] | LC-MS (ESI⁺) m/z 320.3 (M + H)⁺. ¹H NMR (400 MHz, CDCl₃) δ 7.19 − 6.85 (m, 2H), 6.85 − 6.70 (m, 1H), 4.20 − 3.95 (m, 4H), 3.58 − 3.06 (m, 1H), 3.04 − 2.62 (m, 1H), 2.51 − 2.26 (m, 3H), 2.23 − 2.15 (m, 1H), 2.14 − 1.98 (m, 2H), 1.97 (br d, J = 1.0 Hz, 3H), 1.72 − 1.51 (m, 3H), 1.45 (t, J = 6.8 Hz, 6H), 1.33 − 1.16 (m, 2H). |

Compounds listed below could also be prepared from the appropriate ketone starting material according to a procedure similar to that described above in Example 3:

| | | | | | | |
|---|---|---|---|---|---|---|
| 0098 | 0501 | 0701 | 0713 | 0719 | 0725 | 0731 |
| 0737 | 0743 | 0749 | 0767 | 0773 | 0779 | 0785 |
| 0791 | 0797 | 1703 | 1709 | 1721 | 1727 | 1733 |
| 1739 | 1745 | 1751 | 1757 | 1769 | 1775 | 1781 |
| 1787 | 1793 | 1799 | 2705 | 2711 | 2717 | 2723 |
| 2729 | 2735 | 2741 | 2747 | 2753 | 2759 | 2765 |
| 0801 | 0807 | 0813 | 0819 | 0837 | | |

### Example 4: Synthesis of (3aS,6S,7aS)-3a-(3,4-dimethoxyphenyl)-1-methyloctahydro-1H-indol-6-ol ((−)-6-epimesembranol, 0019)

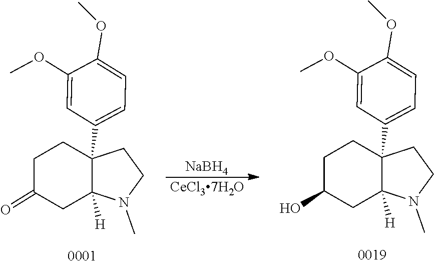 [SMILES: COc1ccc([C@@]23CCC(=O)C[C@@H]2N(C)CC3)cc1OC ; "NaBH4" ; COc1ccc([C@@]23CC[C@H](O)C[C@@H]2N(C)CC3)cc1OC]

To a solution of 0001 (2.00 g, 6.91 mmol) and CeCl₃·7H₂O (3.09 g, 8.29 mmol, 788 uL) in MeOH (80 mL) was added NaBH₄ (1.57 g, 41.4 mmol). The mixture was allowed to stir at 0° C. for 2 hr. The reaction mixture was added into 50 mL NH₄Cl aqueous solution, the organic and aqueous layers were separated, and the aqueous solution was extracted with DCM (50 mL×3). The organic solutions were combined, dried over anhydrous Na₂SO₄, filtered and concentrated under reduced pressure. The residue was purified by prep-HPLC (column: Welch Xtimate C18 150*25 mm*5 um; mobile phase: [water (NH₃H₂O)-ACN]; B %: 28%-58%, 8 min) to give 0019 (730 mg, 37%) as white oil. ¹H NMR (400 MHz, CDCl₃) δ 6.95-6.88 (m, 2H), 6.86-6.80 (m, 1H), 3.95 (s, 1H), 3.90 (d, J=6.8 Hz, 6H), 3.46-3.35 (m, 1H), 2.93 (s, 1H), 2.50 (s, 3H), 2.45-2.29 (m, 2H), 2.19 (dd, J=2.4, 14.9 Hz, 1H), 2.01-1.82 (m, 2H), 1.79-1.72 (m, 1H), 1.70-1.59 (m, 3H), 1.44 (tt, J=2.8, 13.6 Hz, 1H).

Compounds in the table below were prepared from the appropriate ketone starting material according to a procedure similar to that described above in Example 4:

| ID | Structure | Characterization |
|---|---|---|
| 2700 | 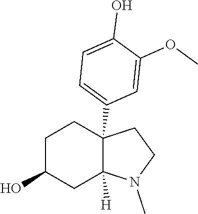 [SMILES: COc1cc([C@@]23CC[C@H](O)C[C@@H]2N(C)CC3)ccc1O] | LC-MS (ESI⁺) m/z 278.2 (M + H)⁺. ¹H NMR (400 MHz, CD₃OD) δ 6.94 (d, J = 1.8 Hz, 1H), 6.87 − 6.82 (m, 1H), 6.80 − 6.73 (m, 1H), 3.97 − 3.88 (m, 1H), 3.87 − 3.82 (m, 3H), 3.46 − 3.37 (m, 1H), 3.07 (br s, 1H), 2.69 − 2.40 (m, 4H), 2.23 (dt, J = 3.6, 14.2 Hz, 1H), 2.17 − 2.09 (m, 1H), 2.05 − 1.96 (m, 3H), 1.80 − 1.60 (m, 2H), 1.55 − 1.41 (m, 1H). |

Compounds listed below could be prepared from the appropriate ketone starting material according to a procedure similar to that described above in Example 4:

| | | | | | | |
|---|---|---|---|---|---|---|
| 0097 | 0502 | 0702 | 0714 | 0720 | 0726 | 0732 |
| 0738 | 0744 | 0750 | 0756 | 0762 | 0768 | 0774 |
| 0780 | 0786 | 0792 | 0798 | 1704 | 1710 | 1716 |
| 1722 | 1728 | 1734 | 1740 | 1746 | 1752 | 1758 |
| 1764 | 1770 | 1776 | 1782 | 1788 | 1794 | 2706 |
| 2712 | 2718 | 2724 | 2730 | 2736 | 2742 | 2748 |
| 2754 | 2760 | 2766 | 0802 | 0814 | 0820 | 0826 |
| 0832 | 0838 | 0850 | | | | |

### Example 5: Synthesis of rac-(3aR,6S,7aS)-3a-(3,4-dimethoxyphenyl)-1-methyl-2,3,3a,6,7,7a-hexahydro-1H-indol-6-ol (0025) and rac-(3aR,6R,7aS)-3a-(3,4-dimethoxyphenyl)-1-methyl-2,3,3a,6,7,7a-hexahydro-1H-indol-6-ol (0026)

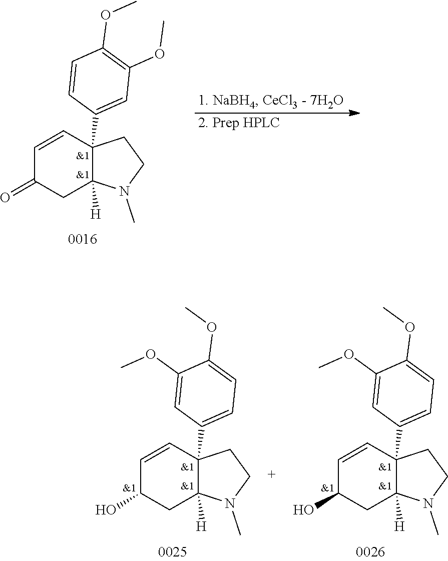 [SMILES: COc1ccc([C@@]23C=CC(=O)C[C@@H]2N(C)CC3)cc1OC ; COc1ccc([C@@]23C=C[C@@H](O)C[C@@H]2N(C)CC3)cc1OC ; COc1ccc([C@@]23C=C[C@H](O)C[C@@H]2N(C)CC3)cc1OC]

A mixture of 0016 (300 mg, 1.04 mmol), NaBH₄ (240 mg, 6.37 mmol) and CeCl₃·7H₂O (466 mg, 1.25 mmol, 119 uL) in MeOH (8 mL) was degassed and purged with N₂ 3 times. The reaction mixture was allowed to stir at 0° C. for 2 hr under an atmosphere of N₂ and then poured into NH₄Cl (5 mL) aqueous solution. The aqueous solution extracted with DCM (10 mL×3). The organic solutions were combined, dried over anhydrous Na₂SO₄, filtered and concentrated under reduced pressure. The residue was purified by prep-HPLC (column: Welch Xtimate C18 150*25 mm*5 um; mobile phase: [water (NH₃H₂O)-ACN]; B %: 25%-55%, 8 min) to give 0025 (80 mg, 27%, peak 1) as yellow solid and 0026 (100 mg, 33%, peak 2) as yellow solid.

0025: LC-MS (ESI⁺) m/z 290.2 (M+H)^{+ 1}H NMR (400 MHz, CDCl₃) δ 6.94-6.88 (m, 2H), 6.84-6.79 (m, 1H), 5.86 (d, J=10.0 Hz, 1H), 5.72 (td, J=1.6, 10.0 Hz, 1H), 4.36 (dd, J=4.8, 9.6 Hz, 1H), 3.89 (d, J=8.4 Hz, 6H), 3.26 (t, J=8.4 Hz, 1H), 2.41 (d, J=10.0 Hz, 1H), 2.37 (s, 3H), 2.31 (d, J=4.0 Hz, 1H), 2.19-2.10 (m, 1H), 2.10-2.02 (m, 1H), 1.47-1.38 (m, 3H).

0026: LC-MS (ESI⁺) m/z 290.3 (M+H)^{+ 1}H NMR (400 MHz, CDCl₃) δ 6.89-6.80 (m, 3H), 6.14 (dd, J=5.2, 10.0 Hz, 1H), 5.76 (d, J=10.0 Hz, 1H), 4.04 (d, J=2.8 Hz, 1H), 3.88 (d, J=5.2 Hz, 6H), 3.33 (dt, J=3.2, 8.8 Hz, 1H), 2.58 (s, 1H), 2.49 (q, J=9.2 Hz, 1H), 2.42 (s, 3H), 2.39-2.35 (m, 1H), 2.18-2.08 (m, 2H), 1.68-1.64 (m, 1H), 1.64-1.60 (m, 1H).

### Example 6: SFC Separation of 0020 and 0023

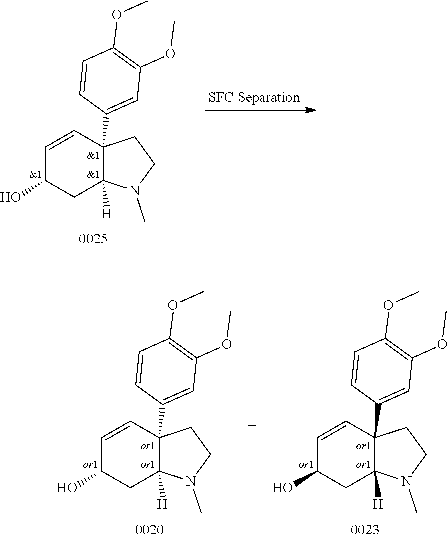 [SMILES: COc1ccc([C@@]23C=C[C@@H](O)C[C@@H]2N(C)CC3)cc1OC ; COc1ccc([C@@]23C=C[C@@H](O)C[C@@H]2N(C)CC3)cc1OC ; COc1ccc([C@]23C=C[C@H](O)C[C@H]2N(C)CC3)cc1OC]

Compound 0025 (80 mg) peak 1 was separated by SFC (column: DAICEL CHIRALCEL OJ (250 mm*30 mm, 10 um); mobile phase: [0.1% NH₃H₂O EtOH]; B %: 15%-15%, 2.5; 35 min) to give 0020 (30 mg, 37%) as white oil and 0023 (30 mg, 37%) as white oil.

0020: LC-MS (ESI+) m/z 290.3 (M+H)⁺; ¹H NMR (400 MHz, CDCl₃) δ 6.95-6.87 (m, 2H), 6.82 (d, J=8.4 Hz, 1H), 5.86 (d, J=10.0 Hz, 1H), 5.72 (d, J=10.0 Hz, 1H), 4.40-4.32 (m, 1H), 3.89 (d, J=8.4 Hz, 6H), 3.26 (t, J=8.4 Hz, 1H), 2.42 (d, J=9.2 Hz, 1H), 2.37 (s, 3H), 2.36-2.28 (m, 2H), 2.20-2.02 (m, 2H), 1.50 (d, J=4.8 Hz, 1H), 1.48-1.39 (m, 1H).

0023: LC-MS (ESI+) m/z 290.3 (M+H)⁺; ¹H NMR (400 MHz, CDCl₃) δ 6.94-6.88 (m, 2H), 6.84-6.80 (m, 1H), 5.86 (d, J=10.0 Hz, 1H), 5.72 (td, J=1.6, 10.0 Hz, 1H), 4.40-4.33 (m, 1H), 3.89 (d, J=8.4 Hz, 6H), 3.26 (t, J=8.4 Hz, 1H), 2.46-2.39 (m, 1H), 2.37 (s, 3H), 2.35-2.28 (m, 2H), 2.19-2.11 (m, 1H), 2.11-2.01 (m, 1H), 1.45 (d, J=10.8 Hz, 1H).

Compounds listed below could be prepared from the appropriate ketone starting material according to a procedure similar to that described above in Example 5 followed by Example 6:

| | | | | | | |
|---|---|---|---|---|---|---|
| 0098 | 0503 | 0703 | 0709 | 0715 | 0721 | 0727 |
| 0733 | 0739 | 0745 | 0751 | 0755 | 0757 | 0761 |
| 0763 | 0769 | 0775 | 0781 | 0787 | 0793 | 0799 |
| 1705 | 1711 | 1715 | 1717 | 1723 | 1729 | 1735 |
| 1741 | 1747 | 1753 | 1759 | 1765 | 1771 | 1777 |
| 1783 | 1789 | 1795 | 2701 | 2707 | 2713 | 2719 |
| 2725 | 2731 | 2737 | 2743 | 2749 | 2755 | 2761 |
| 2767 | 0803 | 0815 | 0821 | 0825 | 0827 | 0833 |
| 0839 | 0849 | 0851 | 0905 | 0915 | 0916 | 0925 |
| 0933 | 0941 | 0951 | 0961 | 0981 | 0991 | 1909 |
| 1915 | 1921 | 1003 | 1009 | 1015 | 1021 | 1027 |

### Example 7: SFC Separation of 0021 and 0024

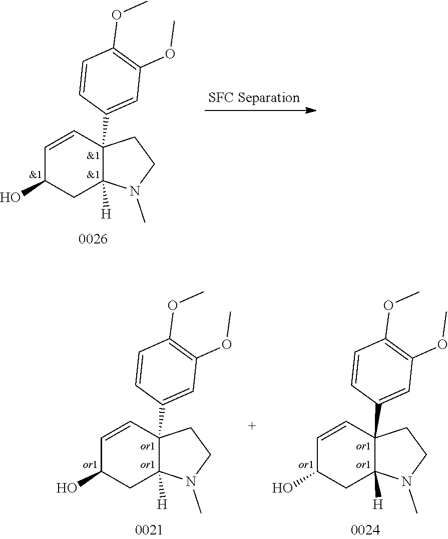 [SMILES: COc1ccc([C@@]23C=C[C@H](O)C[C@@H]2N(C)CC3)cc1OC ; COc1ccc([C@@]23C=C[C@H](O)C[C@@H]2N(C)CC3)cc1OC ; COc1ccc([C@]23C=C[C@@H](O)C[C@H]2N(C)CC3)cc1OC]

Compound 0026 (100 mg) was separated by SFC (column: DAICEL CHIRALCEL OJ (250 mm*30 mm, 10 um); mobile phase: [0.1% NH₃H₂O EtOH]; B %: 15%-15%, 2.5; 35 min) to give 0021 (40 mg, 40%) as white oil and 0024 (50 mg, 50%) as white oil.

0021: LC-MS (ESI+) m/z 290.3 (M+H)⁺; ¹H NMR (400 MHz, CDCl₃) δ 6.83-6.69 (m, 3H), 6.12-6.02 (m, 1H), 5.68 (dd, J=1.2, 10.0 Hz, 1H), 3.95 (dd, J=2.0, 3.2 Hz, 1H), 3.80 (d, J=5.2 Hz, 6H), 3.31-3.14 (m, 1H), 2.50 (s, 1H), 2.45-2.38 (m, 1H), 2.34 (s, 3H), 2.32-2.26 (m, 1H), 2.11-2.01 (m, 2H), 1.61-1.54 (m, 1H).

0024: LC-MS (ESI+) m/z 290.3 (M+H)⁺; ¹H NMR (400 MHz, CDCl₃) δ 6.90-6.76 (m, 3H), 6.15 (dd, J=5.2, 9.9 Hz, 1H), 5.81-5.71 (m, 1H), 4.04 (d, J=3.2 Hz, 1H), 3.88 (d, J=5.2 Hz, 6H), 3.39-3.29 (m, 1H), 2.59 (s, 1H), 2.49 (q, J=9.2 Hz, 1H), 2.43 (s, 3H), 2.40-2.37 (m, 1H), 2.20-2.06 (m, 2H), 1.70-1.65 (m, 1H).

Compounds listed below could be prepared from the appropriate ketone starting material according to a procedure similar to that described above in Example 5 followed by Example 7:

| | | | | | | |
|---|---|---|---|---|---|---|
| 0099 | 0504 | 0704 | 0710 | 0716 | 0722 | 0728 |
| 0734 | 0740 | 0746 | 0752 | 0758 | 0764 | 0770 |
| 0776 | 0782 | 0788 | 0794 | 0906 | 0917 | 0918 |
| 0926 | 0934 | 0942 | 0952 | 0962 | 0982 | 0992 |
| 1910 | 1916 | 1922 | 1004 | 1010 | 1016 | 1022 |
| 1028 | 1700 | 1706 | 1712 | 0834 | 0840 | 0852 |
| 1718 | 1724 | 1730 | 1736 | 1742 | 1748 | 1545 |
| 1760 | 1766 | 1772 | 1778 | 1784 | 1790 | 1796 |
| 2702 | 2708 | 2714 | 2720 | 2726 | 2732 | 2738 |
| 2744 | 2750 | 2756 | 2762 | 2768 | 0804 | 0816 |
| 0822 | 0828 | | | | | |

### Example 8: Synthesis of 1001 and 1002

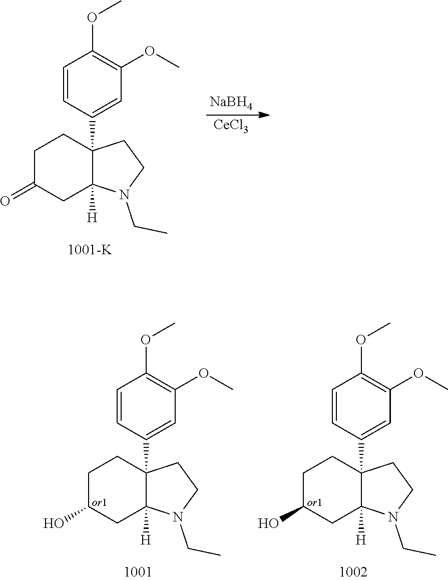

To a solution of 1001-K (50 mg, 164 mol) in MeOH (2.0 mL) was added CeCl₃.7H₂O (73.6 mg, 197 mol). NaBH₄ (37.4 mg, 988 mol) was added to the mixture at 0° C. The mixture was allowed to stir at 0° C. for 1 hr and then poured into ammonium chloride aqueous solution (5.0 mL). The solution was extracted with dichloromethane (30 mL). The organic solution was separated, dried over sodium sulfate, filtered and concentrated in vacuo. The residue was purified by prep-HPLC (column: Waters Xbridge 150*25 mm*5 um; mobile phase: [water (NH₄HCO₃)-ACN]; gradient: 21%-51% B over 10 min) to give 1001 (6.07 mg, 12%) as a yellow gum and 1002 (13.9 mg, 27% yield) as a yellow gum.

1001: LC-MS (ESI⁺) m/z 306.3 (M+H)⁺; ¹H NMR (400 MHz, CD₃Cl) δ 6.96-6.87 (m, 2H), 6.83 (d, J=8.4 Hz, 1H), 4.07 (d, J=3.2 Hz, 1H), 3.90 (d, J=6.8 Hz, 6H), 3.31 (s, 1H), 3.11-2.96 (m, 2H), 2.37-2.28 (m, 1H), 2.19 (d, J=11.6 Hz, 2H), 2.13-2.01 (m, 2H), 1.96-1.88 (m, 1H), 1.86-1.76 (m, 2H), 1.55 (d, J=11.2 Hz, 1H), 1.30-1.21 (m, 1H), 1.14 (t, J=7.2 Hz, 3H).

1002: LC-MS (ESI⁺) m/z 306.3 (M+H)⁺; ¹H NMR (400 MHz, CD₃Cl) δ 6.86-6.78 (m, 2H), 6.77-6.70 (m, 1H), 3.87 (s, 1H), 3.81 (d, J=6.8 Hz, 6H), 3.35 (d, J=8.0 Hz, 1H), 3.14 (d, J=4.0 Hz, 1H), 3.02 (s, 1H), 2.26 (dt, J=3.2, 14.4 Hz, 2H), 2.12 (d, J=14.8 Hz, 2H), 1.92-1.82 (m, 2H), 1.77 (dd, J=8.0, 11.6 Hz, 1H), 1.67 (d, J=13.6 Hz, 1H), 1.60-1.51 (m, 2H), 1.44-1.31 (m, 1H), 1.09 (t, J=7.2 Hz, 3H).

Compounds in the table below were prepared from the appropriate ketone starting material according to a procedure similar to that described above in Example 8:

| ID | Structure | Characterization |
|---|---|---|
| 707 | 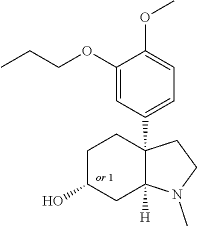 [SMILES: CCCOc1cc([C@@]23CC[C@@H](O)C[C@@H]2N(C)CC3)ccc1OC] | LC-MS (ESI⁺) m/z 320.3 (M + H)⁺. ¹HNMR (400 MHz, CDCl₃) δ 7.03 − 6.69 (m, 3H), 4.12 − 3.92 (m, 3H), 3.86 (s, 3H), 3.24 (dt, J = 4.6, 8.8 Hz, 1H), 2.76 (br s, 1H),2.38 (s, 3H), 2.34 − 2.24 (m, 1H), 2.19 (br d, J = 14.4 Hz, 1H), 2.10 − 1.99 (m, 2H), 1.94 − 1.84 (m, 3H), 1.83 − 1.72 (m, 2H), 1.64 − 1.45 (m, 2H), 1.27 − 1.16 (m, 1H), 1.06 (t, J = 7.2 Hz, 3H). |
| 708 | 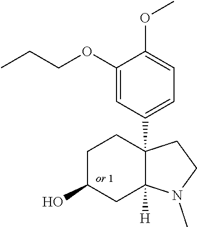 [SMILES: CCCOc1cc([C@@]23CC[C@H](O)C[C@@H]2N(C)CC3)ccc1OC] | LC-MS (ESI⁺) m/z 320.3 (M + H)⁺. ¹HNMR (400 MHz, CDCl₃) δ 6.99 − 6.69 (m, 3H), 4.27 − 3.91 (m, 3H), 3.86 (s, 3H), 3.67 − 3.27 (m, 1H), 3.12 − 2.79 (m, 1H), 2.56 (br s, 3H), 2.48 − 2.39 (m, 1H), 2.38 − 2.15 (m, 2H), 2.14 − 1.80 (m, 5H), 1.80 − 1.64 (m, 2H), 1.49 − 1.35 (m, 1H), 1.06 (t, J = 7.2 Hz, 3H). |

Compounds listed below could be prepared from the appropriate ketone starting material according to a procedure similar to that described above in Example 8:

| | | | | | | |
|---|---|---|---|---|---|---|
| 0901 | 0902 | 0903 | 0904 | 0911 | 0912 | 0913 |
| 0914 | 0921 | 0922 | 0923 | 0924 | 0931 | 0932 |
| 0935 | 0936 | 0939 | 0940 | 0943 | 0944 | 0947 |
| 0948 | 0949 | 0950 | 0957 | 0958 | 0959 | 0960 |
| 0967 | 0968 | 0969 | 0970 | 0977 | 0978 | 0979 |
| 0980 | 0987 | 0988 | 0989 | 0990 | 1907 | 1908 |
| 1913 | 1914 | 1919 | 1920 | 1923 | 1924 | 1927 |
| 1928 | 1929 | 1930 | 1931 | 1932 | 1933 | 1934 |
| 1935 | 1936 | 1937 | 1938 | 1939 | 1940 | 1941 |
| 1942 | 1007 | 1008 | 1013 | 1014 | 1019 | 1020 |
| 1025 | 1026 | 1925 | 1926 | 0971 | 0972 | |

### Example 9: Synthesis of 0070, 0071 and 0072

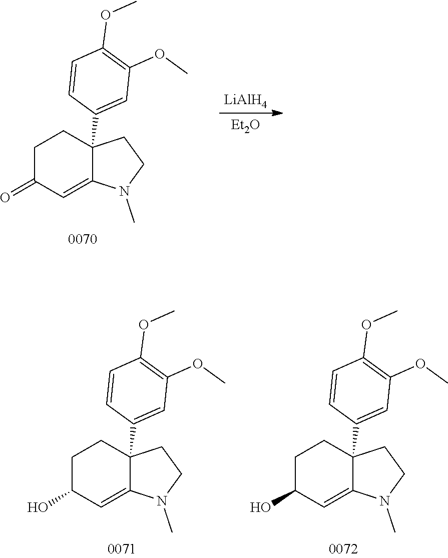 [SMILES: "Et2O" ; COc1ccc([C@@]23CCC(=O)C=C2N(C)CC3)cc1OC ; "LiAlH4" ; COc1ccc([C@@]23CC[C@@H](O)C=C2N(C)CC3)cc1OC ; COc1ccc([C@@]23CC[C@H](O)C=C2N(C)CC3)cc1OC]

Compound 0070 can be prepared according to a process known in the literature (Takano et al. Tet. Lett. 1981, 22, 4479-4482). Reduction of 0071 may be achieved by treatment with lithium aluminium hydride in a solvent such as diethyl ether as reported by Jeff et al. J. Org. Chem. 1970, 35, 3512-3518. The resulting mix of diastereomers may be separated by chiral HPLC to give 0071 and 0072.

Compounds listed below could be prepared from the appropriate ketone starting material according to a procedure similar to that described above in Example 9:

| | | | | | | |
|---|---|---|---|---|---|---|
| 0505 | 0506 | 0705 | 0706 | 0711 | 0712 | 0717 |
| 0718 | 0723 | 0724 | 0729 | 0730 | 0735 | 0736 |
| 0741 | 0742 | 0747 | 0748 | 0753 | 0754 | 0759 |
| 0760 | 0765 | 0766 | 0771 | 0772 | 0777 | 0778 |
| 0783 | 0784 | 0789 | 0790 | 0795 | 0796 | 1701 |
| 1702 | 1707 | 1708 | 1713 | 1714 | 1719 | 1720 |
| 1725 | 1726 | 1731 | 1732 | 1737 | 1738 | 1743 |
| 1744 | 1749 | 1750 | 1755 | 1756 | 1761 | 1762 |
| 1767 | 1768 | 1773 | 1774 | 1779 | 1780 | 1785 |
| 1786 | 1791 | 1792 | 1797 | 1798 | 2703 | 2704 |
| 2709 | 2710 | 2715 | 2716 | 2721 | 2722 | 2727 |
| 2728 | 2733 | 2734 | 2739 | 2740 | 2745 | 2746 |
| 2751 | 2752 | 2757 | 2758 | 2763 | 2764 | 2769 |
| 2770 | 0805 | 0806 | 0817 | 0818 | 0823 | 0824 |
| 0829 | 0830 | 0835 | 0836 | 0841 | 0842 | 0853 |
| 0854 | 0907 | 0908 | 0909 | 0910 | 0919 | 0920 |
| 0927 | 0928 | 0929 | 0930 | 0937 | 0938 | 0945 |
| 0946 | 0953 | 0954 | 0955 | 0956 | 0963 | 0964 |
| 0965 | 0966 | 0973 | 0974 | 0975 | 0976 | 0983 |
| 0984 | 0985 | 0986 | 0993 | 0994 | 0995 | 0996 |
| 1911 | 1912 | 1917 | 1918 | 1005 | 1006 | 1011 |
| 1012 | 1017 | 1018 | 1023 | 1024 | 1029 | 1030 |

### Example 10: Synthesis of 0809 and 0810

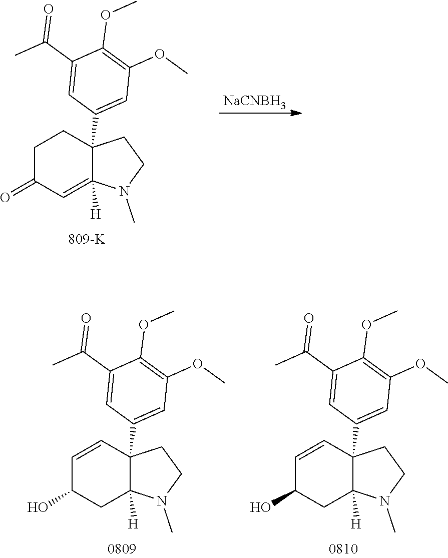

Reduction of 809-K could be achieved by treatment sodium cyanoborohydride in HCl methanol. Alternatively, 809-K could be treated with sulphuric acid HMPA at a temperature between −20° and 40° C. The resulting mix of diastereomers may be separated by chiral HPLC to give 0809 and 0810.

Compounds listed below could be prepared from the appropriate ketone starting material according to a procedure similar to that described above in Example 10:

| | | | | | | |
|---|---|---|---|---|---|---|
| 0807 | 0808 | 0811 | 0812 | 0843 | 0844 | 0845 |
| 0846 | 0847 | 0848 | 0997 | 0998 | 0999 | 1900 |
| 1901 | 1902 | 1903 | 1903 | 1905 | 1906 | |

### Example 11: Synthesis of 851-KR

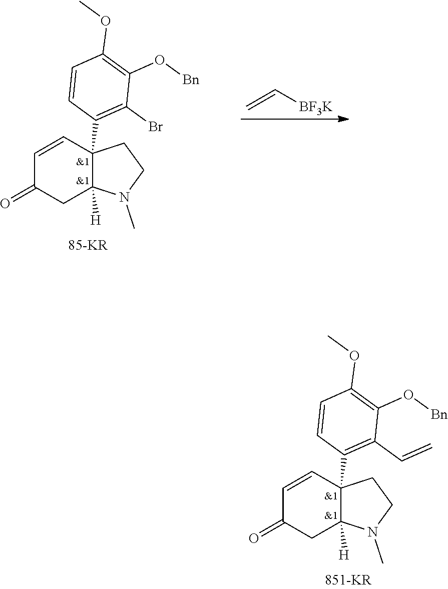

To a mixture of 85-KR (75 mg, 169.55 umol) in H₂O (2 mL) and THE (0.4 mL) was added potassium hydride trifluoro(vinyl)boron (22.71 mg, 169.55 umol), K₃PO₄ (71.98 mg, 339.10 umol) and XPhos Pd G3 (14.35 mg, 16.95 umol). The mixture was allowed to stir at 60° C. for 8 hr under an atmosphere of N₂. The reaction mixture was concentrated and the residue was purified by by prep-TLC (SiO₂, DCM:MeOH=15:1) to give 851-KR (50 mg, 2.94 mmol, 70%) as a yellow solid. ¹H NMR (400 MHz, CDCl₃) δ 7.48 (d, J=7.2 Hz, 1H), 7.40 (d, J=7.2 Hz, 1H), 7.34-7.24 (m, 3H), 7.09-6.90 (m, 2H), 6.80-6.58 (m, 2H), 6.06 (d, J=10.0 Hz, 1H), 5.64 (d, J=17.6 Hz, 1H), 5.21 (d, J=12.4 Hz, 1H), 5.01-4.83 (m, 2H), 3.84-3.78 (m, 3H), 3.32-3.18 (m, 1H), 2.98 (s, 1H), 2.67-2.59 (m, 1H), 2.46-2.34 (m, 2H), 2.27 (d, J=3.6 Hz, 2H), 2.22-2.09 (m, 2H).

### Example 12: Synthesis of 1925-KR

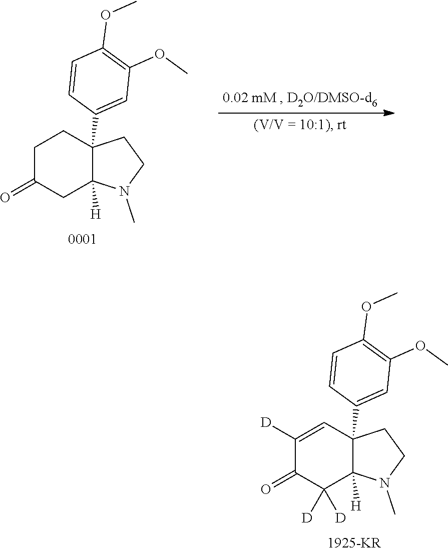 [SMILES: COc1ccc([C@@]23CCC(=O)C[C@@H]2N(C)CC3)cc1OC ; [2H]C1=C[C@@]2(c3ccc(OC)c(OC)c3)CCN(C)[C@H]2C([2H])([2H])C1=O]

A mixture of 001 (100. mg, 348 umol) in D₂O (0.5 mL) and DMSO-d₆ (1 g, 12.80 mmol, 1.00 mL) was degassed and purged with N₂ 3 times, and then the mixture was allowed to stir at 60° C. for 168 h. The mixture was lyophilized to give 1925-KR (72.6 mg, 92% purity) as a yellow solid. LC-MS (ESI⁺) m/z 291.1 (M+H)⁺. ¹H NMR (400 MHz, DMSO-d6) δ=7.10-6.74 (m, 4H), 3.79-3.76 (m, 6H), 3.13 (dt, J=2.8, 8.6 Hz, 1H), 2.72-2.62 (m, 1H), 2.49-2.42 (m, 1H), 2.35-2.35 (m, 1H), 2.29-2.16 (m, 3H), 1.91-1.89 (m, 1H).

Compounds listed below could also be prepared from the appropriate ketone starting material according to a procedure similar to that described above in Example 9:

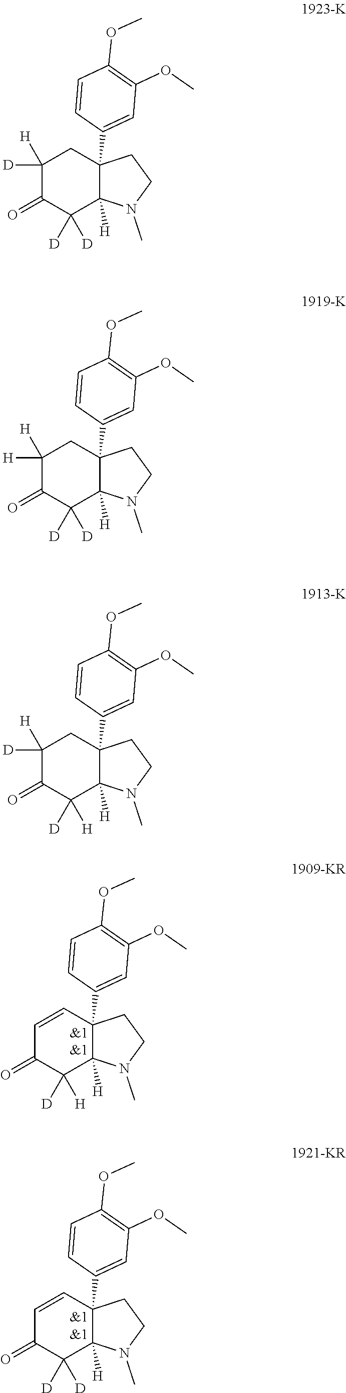 [SMILES: [2H]C1C[C@@]2(c3ccc(OC)c(OC)c3)CCN(C)[C@H]2C([2H])([2H])C1=O ; [2H]C1([2H])C(=O)CC[C@@]2(c3ccc(OC)c(OC)c3)CCN(C)[C@@H]12 ; [2H]C1C[C@@]2(c3ccc(OC)c(OC)c3)CCN(C)[C@H]2C([2H])C1=O ; [2H]C1C(=O)C=C[C@@]2(c3ccc(OC)c(OC)c3)CCN(C)[C@@H]12 ; [2H]C1([2H])C(=O)C=C[C@@]2(c3ccc(OC)c(OC)c3)CCN(C)[C@@H]12]

### Example 13: Synthesis of 1927-K

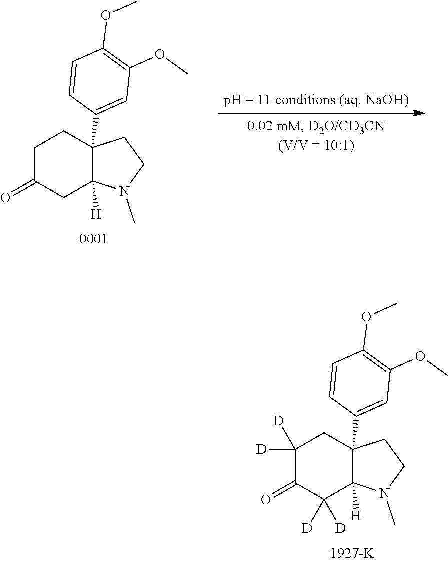 [SMILES: COc1ccc([C@@]23CCC(=O)C[C@@H]2N(C)CC3)cc1OC ; [2H]C1([2H])C[C@@]2(c3ccc(OC)c(OC)c3)CCN(C)[C@H]2C([2H])([2H])C1=O]

A solution of 0001 (100 mg, 345 umol) in D₂O (1.0 mL) and CD₃CN (0.1 mL) was allowed to stir at 25° C. for 2 hours. To the reaction mixture was added NaOH in D₂O until the solution had pH=11. The reaction mixture was filtered using a C18 column and lyophilized to give 1927-K (49 mg) as a colorless oil. LC-MS (ESI⁺) m/z 294.1 (M+H)^{+ 1}H NMR (400 MHz, DMSO-d₆) δ 6.98-6.94 (m, 1H), 7.00-6.94 (m, 1H), 6.92 (s, 1H), 3.76 (d, J=16.0 Hz, 5H), 3.05-2.99 (m, 1H), 2.84 (s, 1H), 2.53 (d, J=2.0 Hz, 1H), 2.25 (s, 5H), 2.11-1.93 (m, 3H).

Compounds listed below could also be prepared from the appropriate ketone starting material according to a procedure similar to that described above in Example 10:

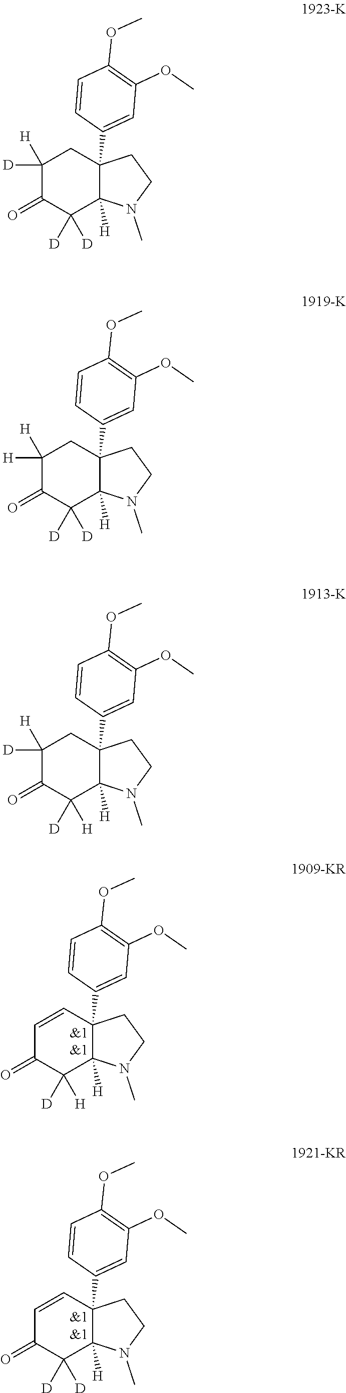 [SMILES: [2H]C1C[C@@]2(c3ccc(OC)c(OC)c3)CCN(C)[C@H]2C([2H])([2H])C1=O ; [2H]C1([2H])C(=O)CC[C@@]2(c3ccc(OC)c(OC)c3)CCN(C)[C@@H]12 ; [2H]C1C[C@@]2(c3ccc(OC)c(OC)c3)CCN(C)[C@H]2C([2H])C1=O ; [2H]C1C(=O)C=C[C@@]2(c3ccc(OC)c(OC)c3)CCN(C)[C@@H]12 ; [2H]C1([2H])C(=O)C=C[C@@]2(c3ccc(OC)c(OC)c3)CCN(C)[C@@H]12]

### Example 14: Synthesis of 845-KR

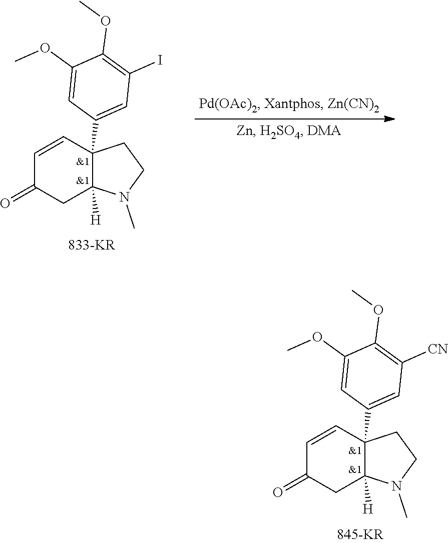 [SMILES: COc1cc([C@@]23C=CC(=O)C[C@@H]2N(C)CC3)cc(I)c1OC ; COc1cc([C@@]23C=CC(=O)C[C@@H]2N(C)CC3)cc(C#N)c1OC]

To a solution of 833-KR (400 mg, 968 umol) and Zn(CN)₂ (182 mg, 1.55 mmol, 98.3 uL) in DMA (5.0 mL) was added Zn (50.6 mg, 774 umol), Xantphos (448 mg, 774 umol), Pd(OAc)₂ (17.3 mg, 77.4 umol) and H₂SO₄ (75.9 mg, 774 umol, 41.2 uL). The mixture was allowed to stir at 80° C. for 2 hr and then poured into aqueous saturated NaHCO₃ (100 mL). The solution was extracted with ethyl acetate (50 mL). The organic solutions were combined, dried over sodium sulfate, filtered and concentrated in vacuo. The residue was purified by prep-HPLC (column: Welch Xtimate C18 150*25 mm*5 um; mobile phase: [water (ammonia hydroxide v/v)-ACN]; B %: 21%-51%, 2 min) to give 845-KR (50 mg, 24%) as a white solid. LC-MS (ESI⁺) m/z 313.2 (M+H)⁺. ¹H NMR (400 MHz, CDCl₃) δ 7.15 (d, J=2.4 Hz, 1H), 7.08 (d, J=2.0 Hz, 1H), 6.73-6.66 (m, 1H), 6.17 (d, J=10 Hz, 1H), 4.05 (s, 3H), 3.92 (s, 3H), 3.42-3.30 (m, 1H), 2.68-2.65 (m, 1H), 2.63-2.52 (m, 2H), 2.49-2.39 (m, 2H), 2.35 (s, 3H), 2.31-2.21 (m, 1H).

### Example 15: Synthesis of 821-KR

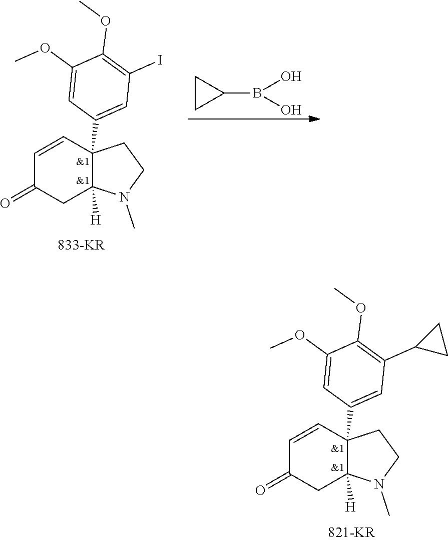

To a solution of 833-KR (150 mg, 363 umol) and cyclopropylboronic acid (155 mg, 1.81 mmol) in Tol (3.0 mL) and H₂O (1.0 mL) was added Na₂CO₃ (115 mg, 1.09 mmol) and Pd(PPh₃)₄ (41.9 mg, 36.3 umol). The mixture was allowed to stir at 90° C. for 12 hr and then poured into water (50 mL). The solution was extracted with ethyl acetate (30 mL). The organic solutions were combined, dried over sodium sulfate, filtered and concentrated in vacuo. The crude product was purified by reversed-phase HPLC (0.1% FA condition) to give 821-KR (70 mg, 57%) as a colorless gum. LC-MS (ESI⁺) m/z 328.2 (M+H)⁺. ¹H NMR (400 MHz, CDCl₃) δ 6.77-6.68 (m, 2H), 6.39 (s, 1H), 6.18 (d, J=10.4 Hz, 1H), 3.88 (s, 6H), 3.48-3.32 (m, 1H), 2.94 (br s, 1H), 2.77-2.68 (m, 1H), 2.65 (br s, 1H), 2.59-2.49 (m, 2H), 2.44 (s, 3H), 2.29-2.20 (m, 2H), 1.01 (br d, J=8.4 Hz, 2H), 0.72-0.58 (m, 2H).

### Example 16: Synthesis of 1931-KR

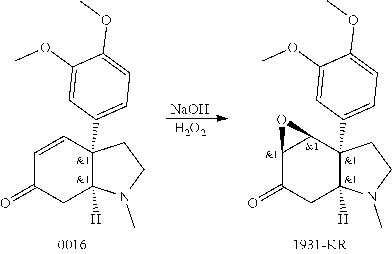

To a solution of 0016 (500 mg, 1.74 mmol) in MeOH (2 mL) was added NaOH (0.5 M, 696 uL) and H₂O₂ (986 mg, 8.70 mmol, 835 μL, 30% purity). The reaction mixture was allowed to stir at 0° C. for 2 hr. The reaction mixture was poured into sodium sulfite solution (50 mL) and wet starch potassium iodide paper was used to test negative (pH<8). The mixture was then poured to the water (30 mL) and extracted with DCM (50 mL). The organic solution was separate, dried over sodium sulfate, filtered and concentrated in vacuo. The residue was purified by prep-HPLC (column: Welch Xtimate C18 150*25 mm*5 um; mobile phase: [water (NH₃H₂O)-ACN]; B %: 12%-42%, 8 min) to give 208 (170 mg, 31.8%) as a yellow solid. LC-MS (ESI⁺) m/z 304.1 (M+H)⁺. ¹H NMR (400 MHz, CDCl₃) δ 6.90-6.77 (m, 2H), 6.71 (s, 1H), 3.87 (s, 6H), 3.56 (d, J=4.0 Hz, 1H), 3.45 (d, J=4.4 Hz, 1H), 3.31-3.22 (m, 1H), 2.99-2.92 (m, 2H), 2.75-2.61 (m, 1H), 2.59-2.44 (m, 3H), 2.30 (s, 3H).

### Example 17: Prep-HPLC Separation of 2753-KR to Give 2753-K and 2753-L

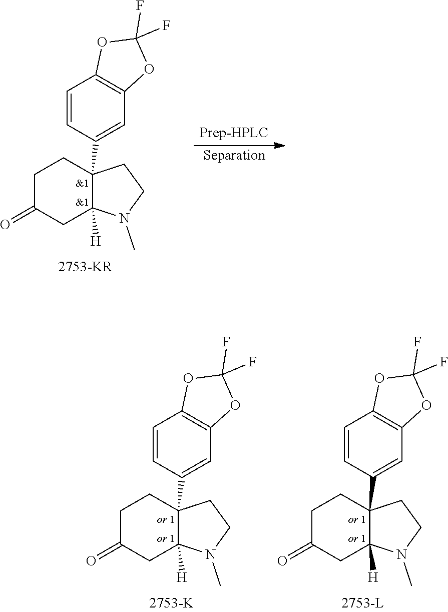 [SMILES: CN1CC[C@]2(c3ccc4c(c3)OC(F)(F)O4)CCC(=O)C[C@H]12 ; CN1CC[C@]2(c3ccc4c(c3)OC(F)(F)O4)CCC(=O)C[C@H]12 ; CN1CC[C@@]2(c3ccc4c(c3)OC(F)(F)O4)CCC(=O)C[C@@H]12]

The enantiomers of 2753-KR were separated by prep-HPLC (column: DAICEL CHIRALPAK AD (250 mm*30 mm, 10 um); mobile phase: [Neu-MeOH]; B %: 25%-25%, C6.0; 54 min) to 2753-K (34 mg, 42%) as yellow oil and 2753-L (35 mg, 53%) as white oil

2753-K: LC-MS (ESI⁺) m/z 310.1 (M+H)⁺. ¹H NMR (400 MHz, CDCl₃) δ 7.18-7.08 (m, 2H), 7.07-7.02 (m, 1H), 3.23-3.11 (m, 1H), 2.93 (s, 1H), 2.67-2.54 (m, 2H), 2.53-2.42 (m, 1H), 2.40-2.29 (m, 4H), 2.26-2.22 (m, 1H), 2.21-2.16 (m, 1H), 2.16-2.07 (m, 3H).

2753-L: LC-MS (ESI⁺) m/z 310.1 (M+H)⁺. ¹H NMR (400 MHz, CDCl₃) δ 7.15-7.08 (m, 2H), 7.06-7.02 (m, 1H), 3.21-3.11 (m, 1H), 2.93 (t, J=3.2 Hz, 1H), 2.60 (dd, J=3.6, 6.1 Hz, 2H), 2.52-2.43 (m, 1H), 2.39-2.29 (m, 4H), 2.28-2.22 (m, 1H), 2.21-2.16 (m, 1H), 2.16-2.06 (m, 3H).

### Example 18: Synthesis of 1935-KR and 1937-KR

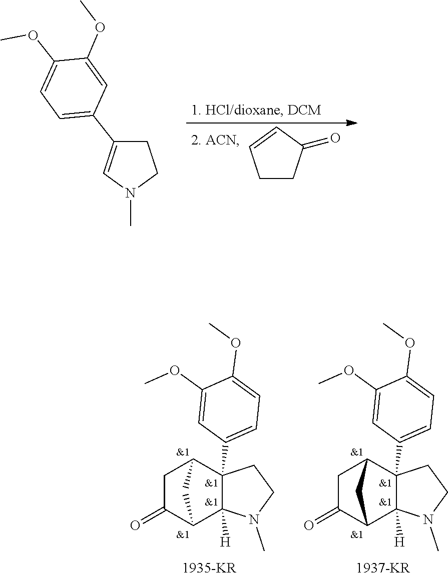 [SMILES: COc1ccc(C2=CN(C)CC2)cc1OC ; O=C1C=CCC1 ; COc1ccc([C@]23CCN(C)[C@H]2[C@@H]2C[C@H]3CC2=O)cc1OC ; COc1ccc([C@]23CCN(C)[C@H]2[C@H]2C[C@@H]3CC2=O)cc1OC]

To a solution of 4-(3,4-dimethoxyphenyl)-1-methyl-2,3-dihydropyrrole (1.00 g, 4.56 mmol) in DCM (10 mL) was added HCl (4M in dioxane, 4.56 mL) and cyclopent-2-en-1-one (449 mg, 5.47 mmol, 458 uL) in ACN (8.0 mL). The reaction mixture was allowed to stir at 90° C. for 16 hr and then aqueous sodium bicarbonate (15 mL) was added to the reaction mixture. The mixture was extracted with EtOAc (20 mL×3). The organic solutions were combined, dried over anhydrous sodium sulfate, filtered and concentrated. The residue was purified by prep-TLC (SiO₂, EtOAc:MeOH=10:1) to give 1935-KR (400 mg, 27%) as a yellow oil and impure 1937-KR. Impure 1937-KR was subjected to additional purification by reversed-phase HPLC (column: Phenomenex C18 150*25 mm*10 um; mobile phase: [water (NH₄HCO₃)-ACN]; B %: 18%-48%, 8 min) to give 212 (120 mg, 8.67%) as a yellow oil.

1935-KR: LC-MS (ESI+) m/z 302.2 (M+H)⁺. ¹H NMR (400 MHz, CDCL₃) δ 6.98-6.89 (m, 2H), 6.83 (d, J=8.4 Hz, 1H), 3.90 (d, J=10.6 Hz, 6H), 3.53 (d, J=5.2 Hz, 1H), 3.06-2.96 (m, 1H), 2.90 (br d, J=4.6 Hz, 1H), 2.80 (br d, J=5.4 Hz, 1H), 2.75-2.64 (m, 1H), 2.63-2.53 (m, 1H), 2.39 (s, 3H), 2.36-2.28 (m, 1H), 2.24-2.12 (m, 1H), 2.04-1.92 (m, 1H), 1.76 (br d, J=10.2 Hz, 2H).

1937-KR: LC-MS (ESI+) m/z 302.1 (M+H)+. ¹H NMR (400 MHz, CDCL₃) δ 6.84-6.75 (m, 1H), 6.71 (s, 2H), 3.86 (d, J=5.6 Hz, 6H), 2.97 (s, 1H), 2.95-2.87 (m, 1H), 2.84 (s, 1H), 2.60 (s, 1H), 2.52 (s, 3H), 2.48-2.37 (m, 2H), 2.16 (d, J=6.4 Hz, 1H), 1.97-1.84 (m, 2H), 1.84-1.79 (m, 1H), 1.59 (br dd, J=5.2, 18.0 Hz, 1H).

Compounds listed below were also prepared from the appropriate starting material according to a procedure similar to that described above in Example 18:

| Starting | | |
|---|---|---|
| Materials | Product | Characterization |
| 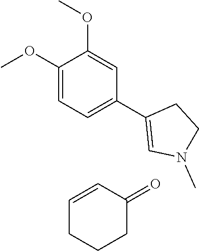 | 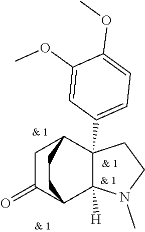 [SMILES: COc1ccc([C@]23CCN(C)[C@H]2[C@H]2CC[C@@H]3CC2=O)cc1OC] | LC-MS (ESI⁺) m/z 316.1 (M + H)⁺. |
| | 1933-KR | |
| 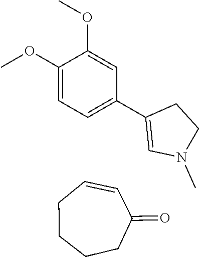 | 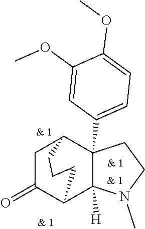 [SMILES: COc1ccc([C@]23CCN(C)[C@H]2[C@@H]2CCC[C@H]3CC2=O)cc1OC] 1939-KR | LC-MS (ESI⁺) m/z 330.2 (M + H)⁺. |
| 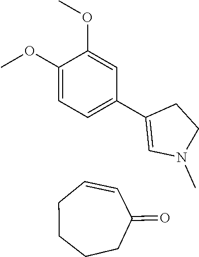 | 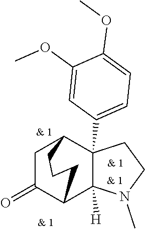 [SMILES: COc1ccc([C@]23CCN(C)[C@H]2[C@H]2CCC[C@@H]3CC2=O)cc1OC] 1941-KR | LC-MS (ESI⁺) m/z 330.2 (M + H)⁺. |

### Example 19: Chiral Separation of 1935-KR to Give 1935-K and 1935-L

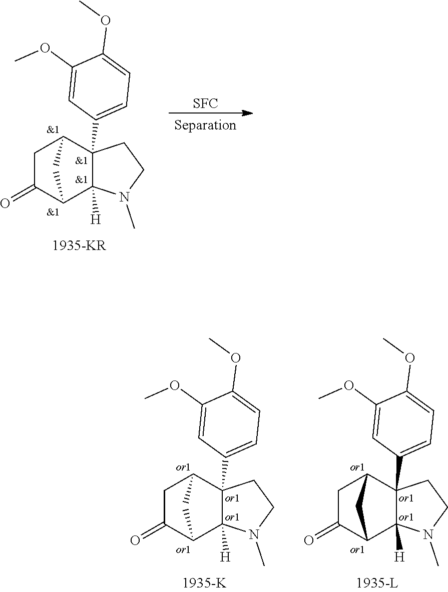

1935-KR was purified by SFC (column: DAICEL CHIRALPAK AS (250 mm*30 mm, 10 um); mobile phase: [Neu-IPA]; B %: 35%-35%, A3.1; 15 min) to give 1935-K and 1935-L, which were each subjected separately to additional purification by prep-HPLC (column: Phenomenex C18 150*25 mm*10 um; mobile phase: [water (NH₄HCO₃)—CN]; %: 22%-52%, min) to give 1935-K (135 mg, 33% yield, 100% purity) as a colorless gum and 1935-L (146 mg, 36% yield, 99% purity) as a yellow gum.

1935-K: LC-MS (ESI+) m/z 302.2 (M+H)⁺. ¹H NMR (400 MHz, CDCL₃) δ 6.93 (s, 2H), 6.83 (d, J=8.4 Hz, 1H), 3.91 (d, J=10.6 Hz, 6H), 3.55-3.50 (m, 1H), 3.04-2.98 (m, 1H), 2.93-2.88 (m, 1H), 2.83-2.77 (m, 1H), 2.74-2.64 (m, 1H), 2.62-2.54 (m, 1H), 2.39 (s, 3H), 2.36-2.29 (m, 1H), 2.20-2.11 (m, 1H), 2.00-1.94 (m, 1H), 1.79-1.70 (m, 2H).

1935-L: LC-MS (ESI+) m/z 302.1 (M+H)⁺. ¹H NMR (400 MHz, CDCL₃) δ 6.98-6.89 (m, 2H), 6.83 (d, J=8.4 Hz, 1H), 3.91 (d, J=10.6 Hz, 6H), 3.59-3.48 (m, 1H), 3.01 (br t, J=8.2 Hz, 1H), 2.90 (br s, 1H), 2.81 (br d, J=4.2 Hz, 1H), 2.75-2.65 (m, 1H), 2.62-2.52 (m, 1H), 2.40 (s, 3H), 2.36-2.29 (m, 1H), 2.20-2.11 (m, 1H), 2.00-1.94 (m, 1H), 1.79-1.70 (m, 2H).

Compounds listed below were also prepared from the appropriate starting material according to a procedure similar to that described above in Example 19:

| Ketone and Separation Conditions | Products | Characterization |
|---|---|---|
| 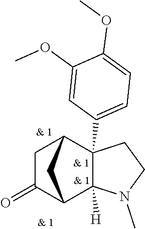 [SMILES: COc1ccc([C@]23CCN(C)[C@H]2[C@H]2C[C@@H]3CC2=O)cc1OC] 1937-KR | Peak 1: 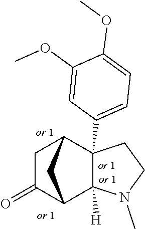 [SMILES: COc1ccc([C@]23CCN(C)[C@H]2[C@H]2C[C@@H]3CC2=O)cc1OC] | LC-MS (ESI⁺) m/z 302.2 (M + H)⁺. ¹H NMR (400 MHz, CDCl₃) δ 6.78 (s, 1H), 6.74 − 6.69 (m, 2H), 3.87 (d, J = 5.4 Hz, 6H), 2.98 (s, 1H), 2.96 − 2.91 (m, 1H), 2.85 (br d, J = 3.2 Hz, 1H), 2.62 (s, 1H), 2.53 (s, 3H), 2.49 − 2.40 (m, 2H), 2.17 (br d, J = 6.2 Hz, 1H), 1.90 (br d, J = 17.2 Hz, 2H), 1.86 − 1.80 (m, 1H), 1.62 (d, J = 5.2 Hz, 1H). |
| column: DAICEL CHIRALPAK IG | 1937-K | |
| (250 mm*30 mm, 10 um); mobile | | |
| phase: [Neu-ETOH]; B%: 35%- | Peak 2: | LC-MS (ESI⁺) m/z 302.2 (M + H)⁺. ¹H |
| 35%, C7; 40 min | | NMR (400 MHz, CDCl₃) δ 6.81 − 6.77 |
| | 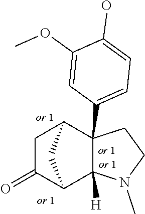 [SMILES: COc1cc([C@@]23CCN(C)[C@@H]2[C@@H]2C[C@H]3CC2=O)ccc1O] | (m, 1H), 6.71 (s, 2H), 3.87 (d, J = 5.4 Hz, 6H), 2.98 (s, 1H), 2.96 − 2.90 (m, 1H), 2.88 − 2.83 (m, 1H), 2.62 (s, 1H), 2.53 (s, 3H), 2.49 − 2.38 (m, 2H), 2.22 − 2.11 (m, 1H), 1.98 − 1.87 (m, 2H), 1.86 − 1.79 (m, 1H), 1.63 − 1.60 (m, 1H). |
| | 1937-L | |
| 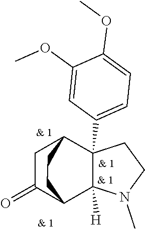 [SMILES: COc1ccc([C@]23CCN(C)[C@H]2[C@H]2CC[C@@H]3CC2=O)cc1OC] 1933-KR | Peak 1: 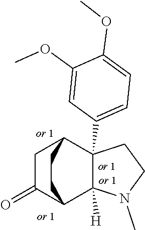 [SMILES: COc1ccc([C@]23CCN(C)[C@H]2[C@H]2CC[C@@H]3CC2=O)cc1OC] | LC-MS (ESI*) m/z 316.2 (M + H)⁺; ¹H NMR (400 MHz, CDCL₃) δ 7.05 − 6.93 (m, 2H), 6.84 (d, J = 8.4 Hz, 1H), 3.92 (d, J = 5.2 Hz, 6H), 3.14 (d, J = 4.4 Hz, 1H), 2.90 − 2.80 (m, 2H), 2.74 − 2.68 (m, 1H), 2.51 (s, 1H), 2.40 (s, 3H), 2.39 − 2.32 (m, 1H), 2.25 − 2.18 (m, 1H), 2.18 − 2.10 (m, 1H), 1.92 (dd, J = 5.6, 12.4 Hz, 1H), 1.75 − 1.68 (m, 1H), 1.61 − 1.53 (m, 1H), 1.38 − 1.27 (m, 2H). |
| column: DAICEL CHIRALCEL OD | 1933-K | |
| (250 mm*30 mm, 10 um); mobile | | |
| phase: [Neu-IPA]; B%: 30%- 30%, 7.4; 59 min | Peak 2: 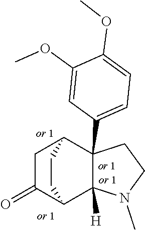 [SMILES: COc1ccc([C@@]23CCN(C)[C@@H]2[C@@H]2CC[C@H]3CC2=O)cc1OC] | LC-MS (ESI⁺) m/z 316.2 (M + H) *; ¹H NMR (400 MHz, CDCL₃) δ 7.04 − 6.92 (m, 2H), 6.84 (d, J = 8.4 Hz, 1H), 3.92 (d, J = 5.6 Hz, 6H), 3.14 (d, J = 4.0 Hz, 1H), 2.90 − 2.81 (m, 2H), 2.74 − 2.68 (m, 1H), 2.51 (s, 1H), 2.41 (s, 3H), 2.39 − 2.32 (m, 1H), 2.25 − 2.18 (m, 1H), 2.18 − 2.09 (m, 1H), 1.93 (dd, J = 5.6, 12.0 Hz, 1H), 1.76 − 1.66 (m, 1H), 1.59 − 1.52 (m, 1H), 1.37 − 1.27 (m, 2H). |
| | 1933-L | |
| 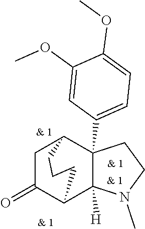 [SMILES: COc1ccc([C@]23CCN(C)[C@H]2[C@@H]2CCC[C@H]3CC2=O)cc1OC] 1939-KR | Peak 1: 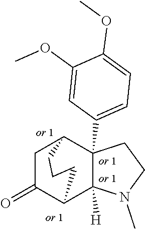 [SMILES: COc1ccc([C@]23CCN(C)[C@H]2[C@@H]2CCC[C@H]3CC2=O)cc1OC] | LC-MS (ESI⁺) m/z 330.30 (M + H)⁺; ¹H NMR (400 MHz, CDCl₃) δ = 6.82 − 6.71 (m, 3H), 3.87 (d, J = 3.0 Hz, 6H), 3.14 − 3.02 (m, 2H), 2.86 (t, J = 6.4 Hz, 1H), 2.57 (dt, J = 7.6, 12.0 Hz, 1H), 2.39 − 2.23 (m, 6H), 2.14 − 2.03 (m, 2H), 1.92 − 1.74 (m, 3H), 1.61 (br dd, J = 6.4, 12.4 Hz, 1H), 1.51 − 1.42 (m, 1H). |
| condition: column: YMC Triart | 1939-K | |
| 30*150 mm*7 um; mobile phase: | | |
| [water(ammonia hydroxide v/v)- MeOH]; B%: 0%-0%, A6.7; 1072 min | Peak 2: 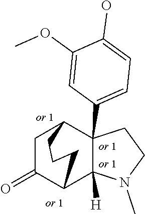 | LC-MS (ESI⁺) m/z 320.20 (M + H)⁺; ¹H NMR (400 MHz, CDCl₃) δ = 6.82 − 6.72 (m, 3H), 3.87 (d, J = 2.8 Hz, 6H), 3.14 − 3.03 (m, 2H), 2.86 (t, J = 6.6 Hz, 1H), 2.62 − 2.52 (m, 1H), 2.38 − 2.21 (m, 6H), 2.13 − 2.03 (m, 2H), 1.92 − 1.74 (m, 3H), 1.65 − 1.59 (m, 1H), 1.48 (br dd, J = 6.4, 13.6 Hz, 1H). |
| | 1939-L | |
| 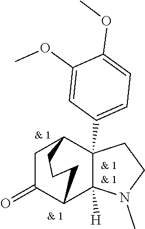 [SMILES: COc1ccc([C@]23CCN(C)[C@H]2[C@H]2CCC[C@@H]3CC2=O)cc1OC] 1941-KR | Peak 1: 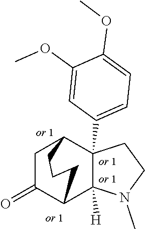 [SMILES: COc1ccc([C@]23CCN(C)[C@H]2[C@H]2CCC[C@@H]3CC2=O)cc1OC] | LC-MS (ESI⁺) m/z 330.30 (M + H)⁺; ¹H NMR (400 MHz, CDCl₃) δ = 6.82 − 6.71 (m, 3H), 3.87 (d, J = 3.0 Hz, 6H), 3.14 − 3.02 (m, 2H), 2.86 (t, J = 6.4 Hz, 1H), 2.57 (dt, J = 7.6, 12.0 Hz, 1H), 2.39 − 2.23 (m, 6H), 2.14 − 2.03 (m, 2H), 1.92 − 1.74 (m, 3H), 1.61 (br dd, J = 6.4, 12.4 Hz, 1H), 1.51 − 1.42 (m, 1H). |
| column: DAICEL CHIRALPAK AS | 1941-K | |
| (250 mm*30 mm, 10 um); mobile | | |
| phase: [Neu-IPA]; B%: 35%-35%, C8.6; 78 min | Peak 2: 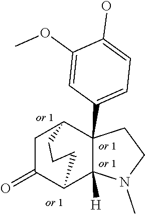 | LC-MS (ESI⁺) m/z 320.20 (M + H)⁺; ¹H NMR (400 MHz, CDCl₃) δ = 6.82 − 6.72 (m, 3H), 3.87 (d, J = 2.8 Hz, 6H), 3.14 − 3.03 (m, 2H), 2.86 (t, J = 6.6 Hz, 1H), 2.62 − 2.52 (m, 1H), 2.38 − 2.21 (m, 6H), 2.13 − 2.03 (m, 2H), 1.92 − 1.74 (m, 3H), 1.65 − 1.59 (m, 1H), 1.48 (br dd, J = 6.4, 13.6 Hz, 1H). |
| | 1941-L | |

### Example A: SERT Inhibition Assay

SERT inhibition was measured using a Neurotransmitter Transportation Fluorescence assay. Briefly, stable 5HHH cells were prepared in a 384 microwell plate. Compounds were prepared by in assay buffer (20 mM HEPES, 0.1% BSA). The compounds were added to the plated cells and incubated for 30 minutes at 37° C. 25 μL of dye solution (Molecular Devices Neurotransmitter Transporter Uptake Assay Kit) was added per well and incubated for 30 minutes at 37° C. The plates were then read on a plate reader.

The results are shown in the Table below as follows: A: IC50</=50 nM or lower; B: 50 nM<IC₅₀</=100 nM; C: 100 nM<IC₅₀</=500 nM; D: 500 nM<IC₅₀</=1 micromolar; E: IC₅₀>1 micromolar.

| | SERT | | |
|---|---|---|---|
| Patent | Assay | | |
| Compound | Potency | IC50 Ratio to | |
| No. | Range | (−) Mesembrine | Structure |
| 0001 | A | 1.00 | 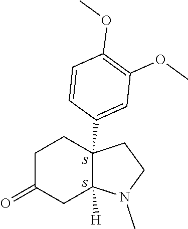 |
| 0002 | B | ≥10 | 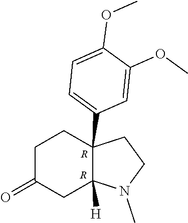 |
| 0016 | C | ≥10 | 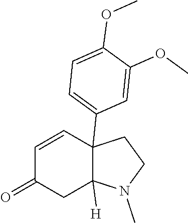 [SMILES: COc1ccc(C23C=CC(=O)CC2N(C)CC3)cc1OC] |
| 0018 | A | ≥1.0 | 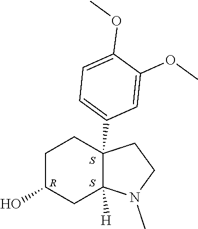 |
| 0019 | A | ≥1.0 | 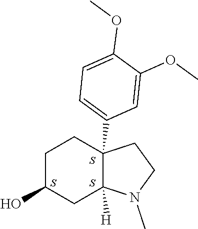 |
| 0020 | C | ≥100 | 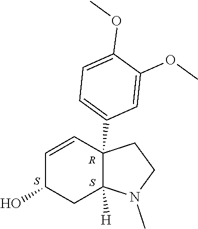 |
| 0021 | C | ≥10 | 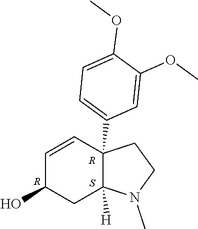 |
| 0023 | C | ≥10 | 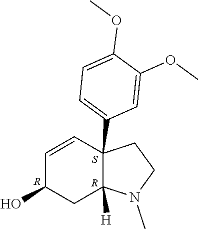 |
| 0024 | C | ≥100 | 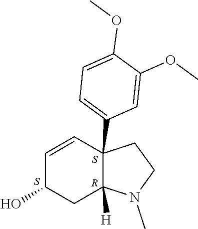 |
| 0707 | B | ≥10 | 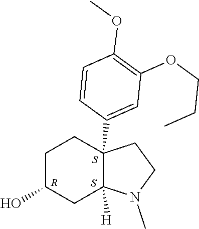 |
| 0708 | A | ≥1.0 | 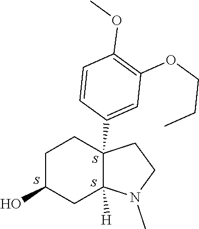 |
| 1001 | E | ≥100 | 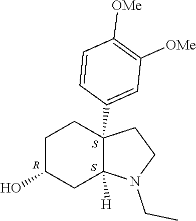 |
| 1002 | D | ≥100 | 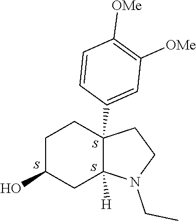 |
| 1763 | A | ≥1.0 | 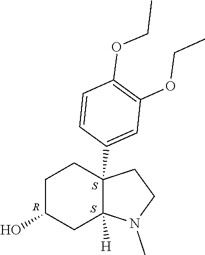 |
| 1764 | A | ≤0.5 | 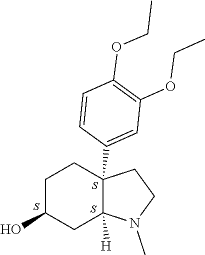 |
| 1930 | C | ≥10 | 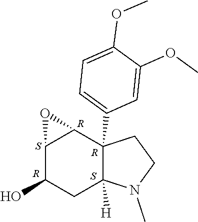 |
| 1932 | C | ≥10 | 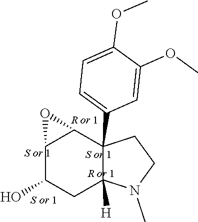 [SMILES: COc1ccc([C@@]23CCN(C)[C@@H]2C[C@H](O)[C@@H]2O[C@@H]23)cc1OC] |
| 1933 | A | ≥1.0 | 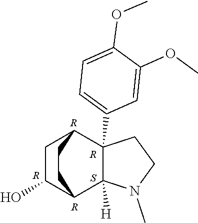 |
| 2700 | A | ≥1.0 | 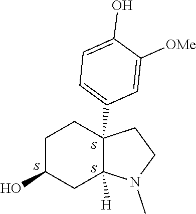 |
| 2723 | A | ≤0.5 | 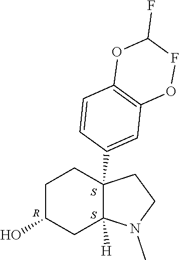 |
| 2724 | A | ≤0.5 | 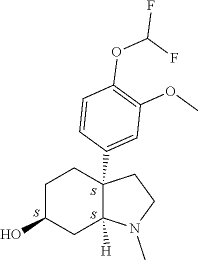 |

1-57. (canceled)

58. A compound of Formula (IA), (IB), or (IC): 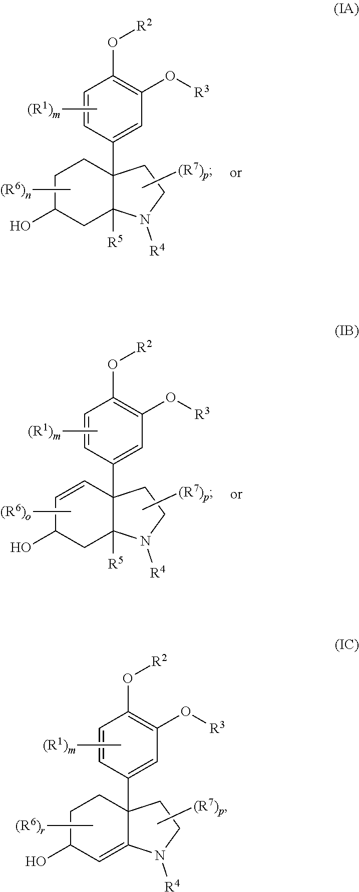
or a pharmaceutically acceptable salt thereof; wherein
each R¹ is independently halo, deuterium, C₁₋₄ alkyl, C₂₋₄ alkenyl, C₂₋₄ alkynyl, C₃₋₈ cycloalkyl, C₃₋₈ cycloalkenyl, 3- to 8-membered heterocyclyl comprising one or more N, O or S heteroatoms, C₅₋₆ aryl, 5- to 6-membered heteroaryl comprising one or more N, O or S heteroatoms, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein C₁₋₄ alkyl, C₂₋₄ alkenyl, and C₂₋₄ alkynyl, are optionally substituted with halo, C₁₋₄ alkyl, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; and wherein C₃₋₈ cycloalkyl, C₃₋₈ cycloalkenyl, 3- to 8-membered heterocyclyl, C₅₋₆ aryl, and 5- to 6-membered heteroaryl are optionally substituted with halo, C₁₋₄ alkyl, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
R² and R³ are independently C₁₋₄ alkyl, H, C₃₋₈ cycloalkyl, C₂₋₄ alkenyl, C₂₋₄ alkynyl, or C₅₋₆ aryl; wherein C₁₋₄ alkyl, C₂₋₄ alkenyl, and C₂₋₄ alkynyl, are optionally substituted with halo, deuterium, C₃₋₈ cycloalkyl, C₅₋₆ aryl, or OR^{a}; and wherein C₃₋₈ cycloalkyl, and C₅₋₆ aryl are optionally substituted with halo, deuterium, C₁₋₄ alkyl, C₁₋₄ haloalkyl or OR^{a}; or
R² and R³ together with the atoms to which they are attached combine to form a 3- to 8-membered heterocyclyl comprising one or more N, O or S heteroatoms, or a 5- to 6-membered heteroaryl comprising one or more N, O or S heteroatoms, wherein the 3- to 8-membered heterocyclyl and 5- to 6-membered heteroaryl are optionally substituted with halo, C₁₋₄ alkyl, C₁₋₄ haloalkyl or OR^{a};
R⁴ is H or C₁₋₄ alkyl, wherein C₁₋₄ alkyl is optionally substituted with halo, deuterium, C₃₋₈ cycloalkyl, C₅₋₆ aryl, —OR^{a}, or —NR^{a}R^{b}; wherein C₅₋₆ aryl is optionally substituted by OR^{a};
R⁵ is H, deuterium or methyl;
each R⁶ is independently C₁₋₄ alkyl, deuterium, C₃₋₈ cycloalkyl, —Si(C₁₋₄ alkyl)₃, —C(O)—(C₁₋₄)-alkyl, C₅₋₆ aryl, 5- to 6-membered heteroaryl comprising one or more N, O or S heteroatoms, or 3- to 8-membered heterocyclyl comprising one or more N, O or S heteroatoms; wherein C₁₋₄alkyl, —Si(C₁₋₄ alkyl)₃, and —C(O)—(C₁₋₄)-alkyl are optionally substituted with halo, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; and wherein C₃₋₈ cycloalkyl, phenyl, 5- to 6-membered heteroaryl, and 3- to 8-membered heterocyclyl are optionally substituted with halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; or two substituents on C₁₋₄ alkyl, C₃₋₈ cycloalkyl, —Si(C₁₋₄ alkyl)₃, —C(O)—(C₁₋₄)-alkyl, phenyl, 5- to 6-membered heteroaryl, and 3- to 8-membered heterocyclyl together with the atoms to which they are attached combine to form C₅₋₆ aryl, C₃₋₈ cycloalkyl, 5- to 6-membered heteroaryl comprising one or more N, O or S heteroatoms, or 3- to 8-membered heterocyclyl comprising one or more N, O or S heteroatoms;
or two R⁶s on different carbon atoms together with the carbon atoms to which they are attached combine to form a C₃₋₈ cycloalkyl, 3- to 8-membered heterocyclyl comprising one or more N, O or S heteroatoms, or a 5- to 6-membered heteroaryl comprising one or more N, O or S heteroatoms, wherein C₃₋₈ cycloalkyl, 3- to 8-membered heterocyclyl, and 5- to 6-membered heteroaryl are optionally substituted with halo, hydroxy, C₁₋₄ alkyl optionally substituted with one or more halo, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
or two R⁶s on a single carbon atom together with the carbon atom to which they are attached combine to form C₃₋₈ cycloalkyl, 3- to 8-membered heterocyclyl comprising one or more N, O or S heteroatoms, or 5- to 6-membered heteroaryl comprising one or more N, O or S heteroatoms, wherein C₃₋₈ cycloalkyl, 3- to 8-membered heterocyclyl, and 5- to 6-membered heteroaryl comprising one or more N, O or S heteroatoms are optionally substituted with halo, hydroxy, C₁₋₄ alkoxy, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each R⁷ is independently deuterium, C₁₋₄ alkyl, C₁₋₄ haloalkyl, —OH, or —NH₂; wherein C₁₋₄ alkyl is optionally substituted with —OR^{a} or —NR^{a}R^{b};
m is 0, 1, 2, or 3;
n is 0, 1, 2, 3, 4, 5, or 6;
o is 0, 1, 2, 3, or 4;
p is 0, 1, 2, 3, or 4;
r is 0, 1, 2, 3, 4, or 5;
R^{a} and R^{b} are independently H, C₁₋₄ alkyl, C₂₋₄ alkenyl, C₃₋₈ cycloalkyl, 3- to 8-membered heterocyclyl comprising one or more N, O or S heteroatoms, C₅₋₆ aryl, or 5- to 6-membered heteroaryl;
or if an instance of R¹ is —NR^{a}R^{b}, then R^{a} and R^{b} may combine with the nitrogen atom to which they are attached to form 3- to 8-membered heterocyclyl or 5- to 6-membered heteroaryl; wherein C₁₋₄ alkyl and C₂₋₄ alkenyl are optionally substituted with halo, hydroxy, C₁₋₄ alkyl, C₁₋₄ alkanol, C₅₋₆ aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; wherein C₃₋₈ cycloalkyl, 3- to 8-membered heterocyclyl, C₅₋₆ aryl, and 5- to 6-membered heteroaryl are optionally substituted with halo, hydroxy, C₁₋₄ alkyl, C₁₋₄ haloalkyl, C₁₋₄ alkanol, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
R^{c} and R^{d} are independently H, C₁₋₄ alkyl, C₂₋₄ alkenyl, C₃₋₈ cycloalkyl, 3- to 8-membered heterocyclyl comprising one or more N, O or S heteroatoms, C₅₋₆ aryl, or 5- to 6-membered heteroaryl comprising one or more N, O or S heteroatoms;
provided the compound is not:

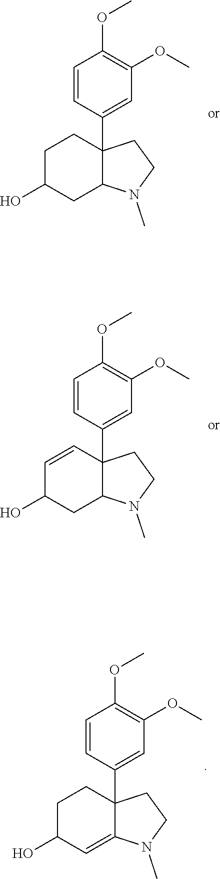 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

59. The compound of claim 58, wherein R⁴ is methyl, R⁵ is H, and each R⁷ is methyl.

60. The compound of claim 59, wherein p is 0.

61. The compound of claim 60, wherein
a. R² and R³ are independently H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl);
b. R² and R³ together with the atoms to which they are attached combine to form a 3- to 8-membered heterocyclyl comprising one or more N, O or S heteroatoms, wherein the 3- to 8-membered heterocyclyl is optionally substituted with halo, C₁₋₄ alkyl, C₁₋₄ haloalkyl or OR^{a}; or
c. R² and R³ together with the atoms to which they are attached combine to form a 5-membered heterocyclyl comprising one or more N, O or S heteroatoms, wherein the 5-membered heterocyclyl is optionally substituted with one or more methyl or fluoro.

62. The compound of claim 61, wherein m is 0 or 1; and R¹ is methyl, methoxy, halo or cyclopropyl.

63. The compound of claim 62, wherein
a. each R⁶ is independently C₁₋₄ alkyl, C₁₋₄ haloalkyl or —C(O)—(C₁₋₄)-alkyl;
b. two R⁶s on different carbon atoms together with the carbon atoms to which they are attached combine to form a C₃₋₈ cycloalkyl or a 3- to 8-membered heterocyclyl comprising one or more N, O or S heteroatoms; or
c. two R⁶s on a single carbon atom together with the carbon atom to which they are attached combine to form C₃₋₈ cycloalkyl optionally substituted with halo, hydroxy, C₁₋₄ alkoxy, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}.

64. The compound of claim 63, wherein
a. two R⁶s on different carbon atoms together with the carbon atoms to which they are attached combine to form an epoxide; or
b. two R⁶s on a single carbon atom together with the carbon atom to which they are attached combine to form a spiro-cyclopropyl.

65. The compound of claim 62, wherein n is 0; and m is 1.

66. The compound of claim 58 wherein the compound is a compound of Formula (IX-A), (IX—B) or (IX—C) 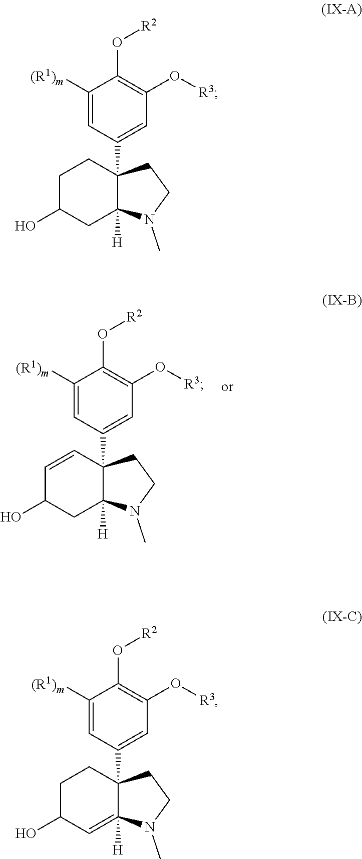
pharmaceutically acceptable salt thereof; wherein
each R¹ is methyl, methoxy, halo or cyclopropyl;
m is 0 or 1;
R² is C₁₋₄ alkyl; and R³ is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); or
R³ is C₁₋₄ alkyl; and R² is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); or
R² and R³ together with the atoms to which they are attached combine to form a 5-membered heterocyclyl comprising one or more N, O or S heteroatoms, wherein the 5-membered heterocyclyl is optionally substituted with one or more methyl or fluoro.

67. The compound of claim 66, wherein
a. R² is C₁₋₄ alkyl; and R³ is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); or
b. R³ is C₁₋₄ alkyl; and R² is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl).

68. A compound of Formula (IA), (IB) or (IC): 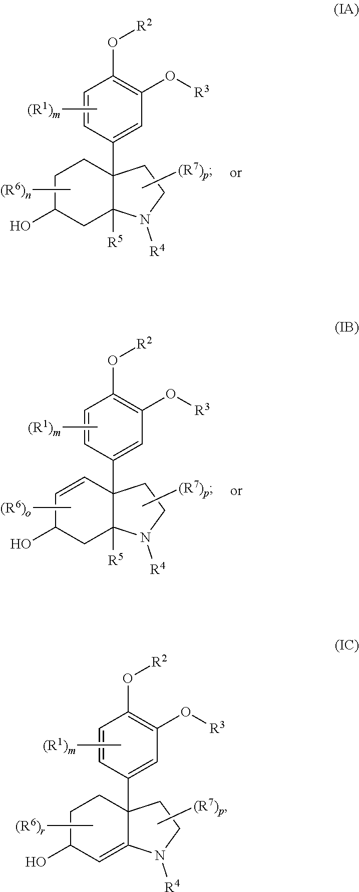
or a pharmaceutically acceptable salt thereof; wherein
each R¹ is methyl, methoxy, halo or cyclopropyl;
R² is C₁₋₄ alkyl and R³ is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); or R³ is C₁₋₄ alkyl and R² is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); or R² and R³ together with the atoms to which they are attached combine to form a 5- to 6-membered heterocyclyl comprising one or more N, O or S heteroatoms, wherein the 5- to 6-membered heterocyclyl is optionally substituted with halo, C₁₋₄ alkyl, C₁₋₄ haloalkyl or OR^{a};
R⁴ is methyl;
R⁵ is H;
each R⁶ is independently C₁₋₄ alkyl, C₁₋₄ haloalkyl or —C(O)—(C₁₋₄-alkyl); or two R⁶s on different carbon atoms together with the carbon atoms to which they are attached combine to form a C₃₋₈ cycloalkyl, or a 3- to 8-membered heterocyclyl comprising one or more N, O or S heteroatoms; or two R⁶s on a single carbon atom together with the carbon atom to which they are attached combine to form a spiro-cyclopropyl;
m is 0 or 1;
n is 0, 1, or 2;
o is 0, 1, or 2;
p is 0;
r is 0, 1, or 2; and
R^{a} is H or C₁₋₄ alkyl;
provided the compound is not:

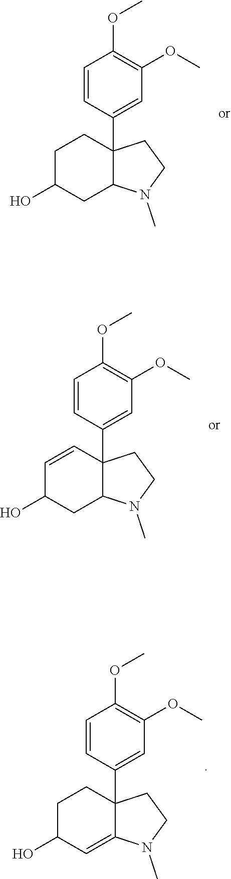 [SMILES: COc1ccc(C23CCC(O)CC2N(C)CC3)cc1OC ; COc1ccc(C23C=CC(O)CC2N(C)CC3)cc1OC ; COc1ccc(C23CCC(O)C=C2N(C)CC3)cc1OC]

69. The compound of claim 68, wherein
a. R² is C₁₋₄ alkyl and R³ is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); or R³ is C₁₋₄ alkyl and R² is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); and
b. n, o and r are 2; and two R⁶s on different carbon atoms together with the carbon atoms to which they are attached combine to form a C₃₋₈ cycloalkyl.

70. The compound of claim 68, wherein the compound is 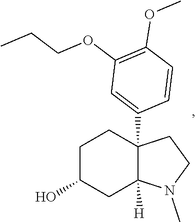 [SMILES: CCCOc1cc([C@@]23CC[C@@H](O)C[C@@H]2N(C)CC3)ccc1OC]
or a pharmaceutically acceptable salt thereof.

71. A compound of Formula (VII-A): 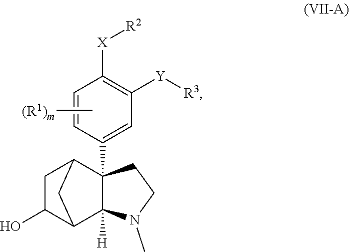
or a pharmaceutically acceptable salt thereof, wherein
X and Y are each independently O or S;
each R¹ is methyl, methoxy, halo or cyclopropyl;
R² is C₁₋₄ alkyl; and R³ is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); or
R³ is C₁₋₄ alkyl; and R² is H, C₁₋₄ alkyl, C₁₋₄ alkyl substituted with one or more fluoro or deuterium, —CH₂CH═CH₂, —CH₂(CCH), C₃₋₆ cycloalkyl, —(CH₂)—(C₃₋₆ cycloalkyl), or —(CH₂)-(phenyl); or
R² and R³ together with the atoms to which they are attached combine to form a 5- to 6-membered heterocyclyl comprising one or more N, O or S heteroatoms, wherein the 5- to 6-membered heterocyclyl is optionally substituted with halo, C₁₋₄ alkyl, C₁₋₄ haloalkyl or OR^{a};
m is 0 or 1;
q is 1, 2 or 3; and
R^{a} is H or C₁₋₄ alkyl.

72. The compound of claim 71, wherein X and Y are each O, m is 0, and R² and R³ are each methyl.

73. The compound of claim 72, wherein q is 1.

74. The compound of claim 72, wherein q is 2.

75. The compound of claim 72, wherein q is 3.

76. A pharmaceutical composition, comprising a compound of claim 58 or a pharmaceutically acceptable salt thereof, and a pharmaceutically acceptable excipient.

77. A method of treating social anxiety disorder, generalized anxiety disorder or depression, the method comprising administering to a mammal in need thereof an effective amount of a compound of claim 58 or a pharmaceutically acceptable salt thereof.
